(19) United States
(12) Patent Application Publication
(10) Pub. No.: US 2026/0048086 A1
(43) Pub. Date: Feb. 19, 2026

(54) METHODS OF DOSING AND ADMINISTRATION OF ENGINEERED ISLET CELLS

(71) Applicant: Sana Biotechnology, Inc., Seattle, WA (US)

(72) Inventor: Sonja SCHREPFER, San Mateo, CA (US)

(21) Appl. No.: 19/376,675

(22) Filed: Oct. 31, 2025

Related U.S. Application Data
(63) Continuation of application No. PCT/US2024/027559, filed on May 2, 2024.
(60) Provisional application No. 63/463,885, filed on May 3, 2023.
(60) Provisional application No. 63/468,217, filed on May 22, 2023.
(60) Provisional application No. 63/580,934, filed on Sep. 6, 2023.
(60) Provisional application No. 63/593,944, filed on Oct. 27, 2023.
(60) Provisional application No. 63/601,142, filed on Nov. 20, 2023.
(60) Provisional application No. 63/551,010, filed on Feb. 7, 2024.

(51) Int. Cl. A61K 35/39; A61K 9/00; A61P 3/10; C12N 5/071

(52) U.S. Cl. CPC A61K 35/39; A61K 9/0019; A61P 3/10; C12N 5/0676; C12N 2510/00

(57) ABSTRACT

Provided herein are methods of dosing engineered islet cells that include functional modified beta cell containing one or more modifications, such as genetic modifications. In some embodiments, the engineered islets are hypoimmunogenic cells. In some embodiments, the one or more modifications reduce or eliminate expression of one or more MHC class I and/or MHC class II human leukocyte antigens and also increase expression of one or more tolerogenic factors, such as CD47. In some embodiments, the subject has a beta cell related disorder, such as diabetes (e.g. Type I diabetes).

### CROSS-REFERENCE TO RELATED APPLICATIONS

This application is a continuation of International Application No. PCT/US2024/027559 filed on May 2, 2024, which claims priority to U.S. Provisional Patent Application No. 63/463,885 filed on May 3, 2023, entitled “METHODS OF DOSING AND ADMINISTRATION OF ENGINEERED ISLET CELLS”, U.S. Provisional Patent Application No. 63/468,217 filed on May 22, 2023, entitled “METHODS OF DOSING AND ADMINISTRATION OF ENGINEERED ISLET CELLS”, U.S. Provisional Patent Application No. 63/580,934 filed on Sep. 6, 2023, entitled “METHODS OF DOSING AND ADMINISTRATION OF ENGINEERED ISLET CELLS”, U.S. Provisional Patent Application No. 63/593,944 filed on Oct. 27, 2023, entitled “METHODS OF DOSING AND ADMINISTRATION OF ENGINEERED ISLET CELLS”, U.S. Provisional Patent Application No. 63/601,142 filed on Nov. 20, 2023, entitled “METHODS OF DOSING AND ADMINISTRATION OF ENGINEERED ISLET CELLS”, and to U.S. Provisional Patent Application No. 63/551,010 filed on Feb. 7, 2024, entitled “METHODS OF DOSING AND ADMINISTRATION OF ENGINEERED ISLET CELLS” the contents of which are incorporated by reference in their entirety.

### INCORPORATION BY REFERENCE OF SEQUENCE LISTING

The present application is being filed along with a Sequence Listing in electronic format. The Sequence Listing is provided as a file entitled 186152009340SeqList.xml created May 2, 2024, which is 99,122 bytes in size. The information in the electronic format of the Sequence Listing is incorporated by reference in its entirety.

### FIELD

In certain aspects, the present disclosure is directed to methods of dosing engineered islet cells that include functional modified beta cell containing one or more modifications, such as genetic modifications. In some embodiments, the engineered islets are hypoimmunogenic cells. In some embodiments, the one or more modifications reduce or eliminate expression of one or more MHC class I and/or MHC class II human leukocyte antigens and also increase expression of one or more tolerogenic factors, such as CD47. In some embodiments, the subject has a beta cell related disorder, such as diabetes (e.g. Type I diabetes).

### SUMMARY

In some embodiments, provided herein is a method of treating or preventing a beta cell disorder in a subject in need thereof, the method comprising administering to the subject a dose of engineered hypoimmunogenic islets, wherein the dose is administered to the subject via intramuscular injection, and wherein the dose is a dose from: A) about 1×10⁷ cells to about 3×10⁸ cells; B) about 1.25×10⁵ cells/kg to about 1.2×10⁷ cells/kg; C) about 6,500 islet equivalents (IEQ) to about 600,000 IEQ; or D) about 80 IEQ/kg to about 24,000 IEQ/kg.

In some embodiments, provided herein is a method of reducing exogenous insulin dependence in a subject having or at risk of having a beta cell disorder, the method comprising administering to the subject a dose of engineered hypoimmunogenic islets, wherein the dose is administered via intramuscular injection, wherein the dose is a dose from: A) about 1×10⁷ cells to about 3×10⁸ cells; B) about 1.25×10⁵ cells/kg to about 1.2×10⁷ cells/kg; C) about 6,500 islet equivalents (IEQ) to about 600,000 IEQ; or D) about 80 IEQ/kg to about 24,000 IEQ/kg, and wherein the amount of exogenous insulin required is less than the amount of exogenous insulin required for a subject treated with non-hypoimmunogenic islets or is less than the amount of exogenous insulin required for untreated subjects that have the beta cell disorder.

In some embodiments, provided herein is a method of promoting insulin independence in a subject having or at risk of having a beta cell disorder, the method comprising administering to the subject a dose of engineered hypoimmunogenic islets, wherein the dose is administered via intramuscular injection, and wherein the dose is a dose from: A) about 1×10⁷ cells to about 3×10⁸ cells; B) about 1.25×10⁵ cells/kg to about 1.2×10⁷ cells/kg; C) about 6,500 Islet equivalents (IEQ) to about 600,000 IEQ; or D) about 80 IEQ/kg to about 24,000 IEQ/kg.

In some embodiments, provided herein is a method of improving graft function in a subject having or at risk of having a beta cell disorder, the method comprising administering to the subject a dose of engineered hypoimmunogenic islets, wherein the dose is administered via intramuscular injection, and wherein the dose is a dose from: A) about 1×10⁷ cells to about 3×10⁸ cells; B) about 1.25×10⁵ cells/kg to about 1.2×10⁷ cells/kg; C) about 6,500 Islet equivalents (IEQ) to about 600,000 IEQ; or D) about 80 IEQ/kg to about 24,000 IEQ/kg.

In some embodiments, provided herein is a method of enhancing engraftment in a subject having or at risk of having a beta cell disorder, the method comprising administering to the subject a dose of engineered hypoimmunogenic islets, wherein the dose is administered via intramuscular injection, and wherein the dose is a dose from: A) about 1×10⁷ cells to about 3×10⁸ cells; B) about 1.25×10⁵ cells/kg to about 1.2×10⁷ cells/kg) about 6,500 Islet equivalents (IEQ) to about 600,000 Islet equivalents (IEQ); D) about 80 IEQ/kg to about 24,000 IEQ/kg.

In some embodiments, provided herein is a method of stabilizing glucose levels in a subject having or at risk of having a beta cell disorder, the method comprising administering to the subject a dose of engineered hypoimmunogenic islets, wherein the dose is administered via intramuscular injection, wherein the dose is a dose from: A) about 1×10⁷ cells to about 3×10⁸ cells; B) about 1.25×10⁵ cells/kg to about 1.2×10⁷ cells/kg; C) about 6,500 islet equivalents (IEQ) to about 600,000 IEQ; or D) about 80 IEQ/kg to about 24,000 IEQ/kg, wherein the glucose levels are stabilized compared to a subject administered an alternative islet therapy or compared to an untreated subject.

In some embodiments, provided herein is a method of stabilizing/increasing c-peptide levels in a subject having or at risk of having a beta cell disorder, the method comprising administering to the subject a dose of engineered hypoimmunogenic islets, wherein the dose is administered via intramuscular injection, and wherein the dose is a dose from: A) about 1×10⁷ cells to about 3×10⁷ cells; B) about 1.25×10⁵ cells/kg to about 1.2×10⁷ cells/kg; C) about 6,500 islet equivalents (IEQ) to about 600,000 IEQ; or D) about 80 IEQ/kg to about 24,000 IEQ/kg, wherein the c-peptide levels are stabilized or increased compared to a subject administered an alternative islet therapy or compared to an untreated subject.

In some embodiments, provided herein is a method of reducing HbA1c levels in a subject having or at risk of having a beta cell disorder, the method comprising administering to the subject a dose of engineered hypoimmunogenic islets, wherein the dose is administered via intramuscular injection, and wherein the dose is a dose from: A) about 1×10⁷ cells to about 3×10⁷ cells; B) about 1.25×10⁵ cells/kg to about 1.2×10⁷ cells/kg) about 6,500 islet equivalents (IEQ) to about 600,000 IEQ; or D) about 80 IEQ/kg to about 24,000 IEQ/kg, wherein the HbA1c levels are reduced compared to a subject administered an alternative islet therapy or compared to an untreated subject.

In some embodiments, provided herein is a method of reducing adverse side effects associated islet cell therapy in a subject having or at risk of having a beta cell disorder, the method comprising i) introducing hypoimmunogenic modification to a population of islet cells comprising beta cells to generate engineered hypoimmunogenic islets, and ii) administering a dose of the engineered hypoimmunogenic islets to a subject having or at risk of having a beta cell disorder, wherein the dose is administered via intramuscular injection, and wherein the dose is a dose from: A) about 1×10⁷ cells to about 3×10⁸ cells; B) about 1.25×10⁵ cells/kg to about 1.2×10⁷ cells/kg; C) about 6,500 islet equivalents (IEQ) to about 600,000 IEQ; or D) about 80 IEQ/kg to about 24,000 IEQ/kg.

In some embodiments, provided herein is a method increasing time in range (TIR) in a subject having or at risk of having a beta cell disorder, the method comprising administering to the subject a dose of engineered hypoimmunogenic islets, wherein the dose is administered via intramuscular injection, and wherein the dose is a dose from: A) about 1×10⁷ cells to about 3×10⁸ cells; B) about 1.25×10⁵ cells/kg to about 1.2×10⁷ cells/kg) about 6,500 islet equivalents (IEQ) to about 600,000 IEQ; or D) about 80 IEQ/kg to about 24,000 IEQ/kg, wherein the TIR is increased compared to a subject administered an alternative islet therapy or compared to an untreated subject.

In some of any embodiments, the method results in reduction in other medication requirements for treating the beta cell disorder, optionally wherein the diabetes medication is insulin. In some of any embodiments, the subject exhibits reduced insulin dependence.

In some of any embodiments, the amount of exogenous insulin is reduced by 10% or more compared to the amount of exogenous insulin required for a subject administered non-hypoimmunogenic islets for treating the beta cell disorder or the amount of exogenous insulin required for untreated subjects that have the beta cell disorder. In some of any embodiments, the amount of insulin is reduced by more than about 20%, about 30%, about 40%, about 50%, about 60%, about 70%, about 75%, about 80% or more.

In some of any embodiments, the method is characterized by the subject meeting one or more of the following criteria: (i) fasting capillary glucose level does not exceed 140 mg/dL (7.8 mmol/L) more than three times in 1 week (based on measuring capillary glucose levels a minimum of 7 times in a seven day period); (ii) 2-hours post-prandial capillary glucose does not exceed 180 mg/dL (10.0 mmol/L) more than three times in 1 week (based on measuring capillary glucose levels a minimum of 21 times in a seven day period); and (iii) evidence of endogenous insulin production defined as fasting or stimulated C-peptide levels >0.5 ng/mL (0.16 pmol/L).

In some of any embodiments, the method results in the subject exhibiting insulin-independence. In some of any embodiments, the subject exhibits insulin-independence for a period of greater than one month, greater than two months, greater than three months, greater than four months, greater than 5 months, greater than 6 months, greater than 7 months, greater than 8 months, greater than 9 months, greater than 10 months, greater than 11 months or greater than 12 months. In some of any embodiments, the subject exhibits insulin-independence for a period of at least 1 year.

In some of any embodiments, the subject is able to titrate off insulin therapy for at least 1 week and meet one or more of the following criteria: (i) fasting capillary glucose level does not exceed 140 mg/dL (7.8 mmol/L) more than three times in 1 week (based on measuring capillary glucose levels a minimum of 7 times in a seven day period); (ii) 2-hours post-prandial capillary glucose does not exceed 180 mg/dL (10.0 mmol/L) more than three times in 1 week (based on measuring capillary glucose levels a minimum of 21 times in a seven day period); and (iii) evidence of endogenous insulin production defined as fasting or stimulated C-peptide levels >0.5 ng/mL (0.16 pmol/L).

In some of any embodiments, the subject is able to titrate off insulin therapy for the period and meet one or more of the following criteria: (i) fasting capillary glucose level does not exceed 140 mg/dL (7.8 mmol/L) more than three times in 1 week (based on measuring capillary glucose levels a minimum of 7 times in a seven day period); (ii) 2-hours post-prandial capillary glucose does not exceed 180 mg/dL (10.0 mmol/L) more than three times in 1 week (based on measuring capillary glucose levels a minimum of 21 times in a seven day period); and (iii) evidence of endogenous insulin production defined as fasting or stimulated C-peptide levels >0.5 ng/mL (0.16 pmol/L). In some of any embodiments, the subject is characterized by at least two of (i)-(iii). In some of any embodiments, the subject is characterized by each of (i)-(iii).

In some of any embodiments, the method is characterized by the subject meeting one or more of the following: a) Peak c-peptide >0.20 nmol/1 (as assessed by mixed meal tolerance test); b) Non-fasting c-peptide >0.10 nmol/1 (as assessed by mixed meal tolerance test); c) Daily exogenous insulin requirement <0.25 U/kg; d) Daily exogenous insulin requirement=0 U/kg; e) Decrease in exogenous insulin requirement (per kg body weight); f) Decrease in HbA1c (per kg body weight); g) Decrease in glucose variability (stabilization); h) Decrease in duration of hypoglycemia and/or hyperglycemia (improved euglycemia); i) Glycemic control HbA1c<6.5% (48 mmol/mol); and j) Glycemic control HbA1c<7.0% (53 mmol/mol). In some of any embodiments, the method is characterized by the subject meeting 2, 3, 4, 5, 6, 7, 8, 9 or 10 of a)-j). In some of any embodiments, the method is characterized by the subject meeting each of a)-j).

In some of any embodiments, the engineered hypoimmunogenic islets comprise modifications that: (a) inactivate or disrupt one or more alleles of: (i) one or more major histocompatibility complex (MHC) class I molecules or one or more molecules that regulate expression of the one or more MHC class I molecules, and/or (ii) one or more MHC class II molecules or one or more molecules that regulate expression of the one or more MHC class II molecules; and/or (b) increase expression of one or more tolerogenic factors, wherein the increased expression is relative to a control or wild-type islet that does not comprise the modifications.

In some of any embodiments, the engineered hypoimmunogenic islets comprise engineered beta islet cells. In some of any embodiments, the engineered hypoimmunogenic islets further comprises additional engineered islet cells, wherein the additional engineered islet cells comprise alpha cells and/or delta cells. In some of any embodiments, the additional engineered islet cells comprises cells that comprises the same modifications of the engineered beta islet cells.

In some of any embodiments, at least 20%, at least 25%, at least 30%, at least 35%, at least 40%, at least 45%, at least 50%, at least 55%, at least 60%, at least 65%, or at least 70% of the cells in the engineered hypoimmunogenic islets comprise engineered beta islet cells. In some of any embodiments, at least 60% of the cells in the engineered hypoimmunogenic islets comprise engineered beta islet cells. In some of any embodiments, the engineered hypoimmunogenic islets is an islet cluster. In some of any embodiments, the engineered hypoimmunogenic islets is engineered from primary islets. In some of any embodiments, the primary islets are from a pancreas. In some of any embodiments, the primary islets are from a human subject. In some of any embodiments, the primary islets are from an animal subject. In some of any embodiments, the primary islets are porcine, bovine or ovine.

In some of any embodiments, the primary islets are from a donor subject that is not suspected of having a beta cell related disorder. In some of any embodiments, the donor is a cadaver. In some of any embodiments, the engineered hypoimmunogenic islets are ABO blood group type O. In some of any embodiments, the engineered hypoimmunogenic islets are Rhesus factor negative (Rh-).

In some of any embodiments, the engineered hypoimmunogenic islets are differentiated from a stem cell. In some of any embodiments, the stem cell is selected from the group consisting of a pluripotent stem cell (PSC), an induced pluripotent stem cell (iPSC), an embryonic stem cell, a hematopoietic stem cell, a mesenchymal stem cell, an endothelial stem cell, an epithelial stem cell, an adipose stem cell, a germline stem cell, a lung stem cell, a cord blood stem cell, and a multipotent stem cell. In some of any embodiments, the stem cell is an induced pluripotent stem cell (iPSC), mesenchymal stem cell (MSC), hematopoietic stem cell (HSC), or embryonic stem cell (ESC). In some of any embodiments, the stem cell is a pluripotent stem cell (PSC).

In some of any embodiments, the beta cell disorder is a metabolic disorder. In some of any embodiments, the metabolic disorder is selected from the group consisting of: familial hypercholesterolemia, Gaucher disease, Hunter syndrome, Krabbe disease, maple syrup urine disease, metachromatic leukodystrophy, mitochondrial encephalopathy, lactic acidosis, stroke-like episodes (MELAS), Niemann-Pick disease, phenylketonuria (PKU), porphyria, Tay-Sachs disease, Wilson's disease, Type I diabetes, Type II diabetes, obesity, hypertension, dyslipidemia, and carbohydrate intolerance. In some of any embodiments, the beta cell disorder is diabetes. In some of any embodiments, the beta cell disorder is Type I diabetes.

In some of any embodiments, the subject to be treated is characterized by one or more of the following: type 1 diabetes for more than 5 years, C-peptide negative (or <0.01 nmol/1) in response to mixed meal tolerance test (MMTT), positive for antibodies to either GAD or IA2, HbA1c≥70 mmol/mol, and an exogenous insulin requirement <1 U/kg.

In some of any embodiments, the dose of engineered hypoimmunogenic islets comprises a pharmaceutically acceptable carrier. In some of any embodiments, the pharmaceutically acceptable carrier is a buffered aqueous solution. In some of any embodiments, the buffered aqueous solution is saline. In some of any embodiments, the dose is administered to the subject intravenously. In some of any embodiments, when the dose is administered intravenously, it is administered intravenously via the portal vein. In some of any embodiments, the dose is administered to the subject via a kidney capsule. In some of any embodiments, the dose is administered to the subject subcutaneously.

In some of any embodiments, the engineered hypoimmunogenic islets are administered to the subject intramuscularly. In some of any embodiments, the intramuscular administration is via the intramuscular space of the forearm. In some of any embodiments, the engineered hypoimmunogenic islets are administered to the upper arm, hip, thigh or buttocks.

In some of any embodiments, the dose is administered to the liver, kidney, spleen, muscle, subcutaneous tissue or white adipose tissue of the subject. In some of any embodiments, the dose is administered to the liver, muscle or white adipose tissue of the subject. In some of any embodiments, the white adipose tissue is omentum.

In some of any embodiments, the dose comprises administration of one or more further doses of the hypoimmunogenic engineered cells.

In some of any embodiments, the one or more further doses of the hypoimmunogenic engineered cells is administered to the subject when, after the initial dose: (a) the subject does not exhibit a reduction in other medication requirements for treating the beta cell disorder, optionally wherein the beta cell disorder medication is insulin; and/or (b) the administered hypoimmunogenic engineered cells are not detected by imaging. In some of any embodiments, the subject does not exhibit reduced insulin dependence after the initial dose.

In some of any embodiments, the one or more further doses of the hypoimmunogenic engineered cells is administered to the subject when, after the initial dose, the subject does not meet one or more of the following criteria: (i) fasting capillary glucose level does not exceed 140 mg/dL (7.8 mmol/L) more than three times in 1 week (based on measuring capillary glucose levels a minimum of 7 times in a seven day period); (ii) 2-hours post-prandial capillary glucose does not exceed 180 mg/dL (10.0 mmol/L) more than three times in 1 week (based on measuring capillary glucose levels a minimum of 21 times in a seven day period); and (iii) evidence of endogenous insulin production defined as fasting or stimulated C-peptide levels >0.5 ng/mL (0.16 pmol/L).

In some of any embodiments, the one or more further doses of the hypoimmunogenic engineered cells is administered to the subject when: (a) the subject does not achieve insulin-independence within a period of time after the initial dose; and/or (b) the subject does not exhibit a reduction in other medication requirements for treating the beta cell disorder within a period of time, optionally wherein the beta cell disorder medication is insulin.

In some of any embodiments, the subject does not achieve insulin-independence for a period of greater than one week, greater than two weeks, greater than three weeks, greater than one month, greater than two months, greater than three months, greater than four months, greater than 5 months, greater than 6 months, greater than 7 months, greater than 8 months, greater than 9 months, greater than 10 months, greater than 11 months or greater than 12 months, optionally wherein the subject does not achieve insulin-independence for a period of 2 weeks. In some of any embodiments, the subject does not achieve insulin-independence for a period of at least 1 year.

In some of any embodiments, the one or more further doses of the hypoimmunogenic engineered cells is administered to the subject when, after the initial dose, the subject is not able to titrate off insulin therapy for at least 1 week and meet one or more of the following criteria: (i) fasting capillary glucose level does not exceed 140 mg/dL (7.8 mmol/L) more than three times in 1 week (based on measuring capillary glucose levels a minimum of 7 times in a seven day period); (ii) 2-hours post-prandial capillary glucose does not exceed 180 mg/dL (10.0 mmol/L) more than three times in 1 week (based on measuring capillary glucose levels a minimum of 21 times in a seven day period); and (iii) evidence of endogenous insulin production defined as fasting or stimulated C-peptide levels >0.5 ng/mL (0.16 pmol/L).

In some of any embodiments, the one or more further doses of the hypoimmunogenic engineered cells is administered to the subject when, after the initial dose, the subject is not able to titrate off insulin therapy for the period and meet one or more of the following criteria: (i) fasting capillary glucose level does not exceed 140 mg/dL (7.8 mmol/L) more than three times in 1 week (based on measuring capillary glucose levels a minimum of 7 times in a seven day period); (ii) 2-hours post-prandial capillary glucose does not exceed 180 mg/dL (10.0 mmol/L) more than three times in 1 week (based on measuring capillary glucose levels a minimum of 21 times in a seven day period); and (iii) evidence of endogenous insulin production defined as fasting or stimulated C-peptide levels >0.5 ng/mL (0.16 pmol/L). In some of any embodiments, the subject is characterized by not meeting at least two of (i)-(iii) or each of (i)-(iii).

In some of any embodiments, the one or more further doses of the hypoimmunogenic engineered cells is administered to the subject when, after the initial dose, the subject does not meet one or more of the following criteria: a) Peak c-peptide >0.20 nmol/1 (as assessed by mixed meal tolerance test); b) Non-fasting c-peptide >0.10 nmol/1 (as assessed by mixed meal tolerance test); c) Daily exogenous insulin requirement <0.25 U/kg; d) Daily exogenous insulin requirement=0 U/kg; e) Decrease in exogenous insulin requirement (per kg body weight); f) Decrease in HbA1c (per kg body weight); g) Decrease in glucose variability (stabilization); h) Decrease in duration of hypoglycemia and/or hyperglycemia (improved euglycemia); i) Glycemic control HbA1c≤6.5% (48 mmol/mol); and j) Glycemic control HbA1c≤7.0% (53 mmol/mol). In some of any embodiments, the one or more further doses is administered to the subject, after the initial dose, if the subject does not meet 2, 3, 4, 5, 6, 7, 8, 9 or 10 of a)-j). In some of any embodiments, the one or more further doses is administered to the subject, after the initial dose, if the subject does not meet each of a)-j).

In some of any embodiments, prior to administering the one or more further doses of engineered hypoimmune islets, the number of the engineered hypoimmunogenic islets from the initial dose are cleared or reduced in the subject. In some of any embodiments, the number of engineered hypoimmunogenic islets are reduced in the subject following administration of an exogenously administered agent to direct targeted death of the engineered hypoimmunogenic islets. In some of any embodiments, the exogenously administered agent activates a suicide gene or safety switch in the engineered cells or recognizes one or more tolerogenic factors on the surface of the engineered hypoimmunogenic islets.

In some of any embodiments, the subject is administered an immunosuppression regimen. In some of any embodiments, the immunosuppression regimen comprises one or more of mycophenolate mofetil (MMF), an anti-CD25 antibody (e.g. basiliximab) and a calcineurin inhibitor (e.g., tacrolimus; FK-506). In some of any embodiments, the immunosuppression regimen comprises administration of Basiliximab (e.g. 2×20 mg iv) followed by Tacrolimus (start dose 0.1 mg/kg/24 h; with target concentration of 10-12) and MMF immunosuppression (500 mg 2×2, dose adjusted thereafter based on AUC). In some of any embodiments, the subject is further administered one or more of the following: CMV prophylaxis Valganciclovir (e.g. 450 mg 2×1), an ulcer prophylaxis with omeprazole (e.g. 20 mg 1×1), TNF-alpha inhibition with etanercept (e.g., 50 mg iv, followed by 25 mg sc on day 3, 7 and 10), and standard antibiotics.

In some of any embodiments, the immunosuppression regimen is administered to the subject only prior to administration of the dose of the engineered hypoimmunogenic islets. In some of any embodiments, the immunosuppression regimen is administered to the subject only 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 days prior to administration of the dose of the engineered hypoimmunogenic islets. In some of any embodiments, the immunosuppression regimen is administered to the subject only 1, 2, 3, 4, 5, 6, 7, 8, 9, 10, 11, 12, 13, 14, 15, 16, 17, 18, 19, 20, 21, 22, 23, 24, 25, 26, 27, or 28 days prior to administration of the dose of the engineered hypoimmunogenic islets. In some of any embodiments, the immunosuppression regimen is administered to the subject only 1, 2, 3, 4, or 5 weeks prior to administration of the dose of the engineered hypoimmunogenic islets. In some of any embodiments, the immunosuppression regimen is administered to the subject only 1, 2, 3, or 4 weeks prior to administration of the dose of the engineered hypoimmunogenic islets. In some of any embodiments, the immunosuppression regimen is administered to the subject only after administration of the dose of the engineered hypoimmunogenic islets. In some of any embodiments, the immunosuppression regimen is administered to the subject only 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 days after administration of the dose of the engineered hypoimmunogenic islets. In some of any embodiments, the immunosuppression regimen is administered to the subject only 1, 2, 3, 4, 5, 6, 7, 8, 9, 10, 11, 12, 13, 14, 15, 16, 17, 18, 19, 20, 21, 22, 23, 24, 25, 26, 27, or 28 days after administration of the dose of the engineered hypoimmunogenic islets. In some of any embodiments, the immunosuppression regimen is administered to the subject only 1, 2, 3, 4, or 5 weeks after administration of the dose of the engineered hypoimmunogenic islets. In some of any embodiments, the immunosuppression regimen is administered to the subject only 1, 2, 3, or 4 weeks after administration of the dose of the engineered hypoimmunogenic islets.

In some of any embodiments, the immunosuppression regimen is administered to the subject intravenously. In some of any embodiments, the immunosuppression regimen is administered to the subject via a kidney capsule. In some of any embodiments, the immunosuppression regimen is administered to the subject orally. In some of any embodiments, the immunosuppression regimen is administered to the subject rectally. In some of any embodiments, the immunosuppression regimen is administered to the subject subcutaneously. In some of any embodiments, the immunosuppression regimen is administered to the subject intramuscularly. In some of any embodiments, the immunosuppression regimen is administered to the forearm of the subject. In some of any embodiments, the immunosuppression regimen is administered to the upper arm, hip, thigh or buttocks. In some of any embodiments, the immunosuppression regimen is administered at least once daily. In some of any embodiments, the immunosuppression regimen is administered as a single regimen per day. In some of any embodiments, the immunosuppression regimen is administered as a divided regimen. In some of any embodiments, the immunosuppression regimen is divided between 2 regimens, 3 regimen

In some of any embodiments, the immunosuppression regimen comprises one or more immunosuppression agents. In some of any embodiments, the one or more immunosuppression agents are administered to the subject prior to administration of the dose of engineered hypoimmunogenic islets. In some of any embodiments, the one or more immunosuppression agents are administered to the subject only prior to administration of a first and/or second administration of the dose of engineered hypoimmunogenic islets. In some of any embodiments, the one or more immunosuppression agents are administered to the subject at least 1, 2, 3, 4, 5, 6, 7, 8, 9, 10, 11, 12, 13, or 14 days prior to administration of the dose of engineered hypoimmunogenic islets. In some of any embodiments, the one or more immunosuppression agents are administered to the subject at least 1 week, 2 weeks, 3 weeks, 4 weeks, 5 weeks, 6 weeks, 7 weeks, 8 weeks, 9 weeks, 10 weeks or more prior to administration of the dose of engineered hypoimmunogenic islets. In some of any embodiments, the one or more immunosuppression agents are administered to the subject after administration of the dose of engineered hypoimmunogenic islets. In some of any embodiments, the one or more immunosuppression agents are administered to the subject only after administration of a first and/or second administration of the dose of engineered hypoimmunogenic islets. In some of any embodiments, the one or more immunosuppression agents are administered to the subject after administration of the dose of engineered hypoimmunogenic islets. In some of any embodiments, the one or more immunosuppression agents are administered to the subject after administration of the dose of engineered hypoimmunogenic islets. In some of any embodiments, the one or more immunosuppression agents are administered to the subject after administration of the dose of engineered hypoimmunogenic islets. In some of any embodiments, the one or more immunosuppression agents are administered to the subject at least 1, 2, 3, 4, 5, 6, 7, 8, 9, 10, 11, 12, 13, or 14 days after administration of the dose of engineered hypoimmunogenic islets. In some of any embodiments, the one or more immunosuppression agents are administered to the subject at least 1 week, 2 weeks, 3 weeks, 4 weeks, 5 weeks, 6 weeks, 7 weeks, 8 weeks, 9 weeks, 10 weeks, or more, after administration of the dose of engineered hypoimmunogenic islets. In some of any embodiments, the one or more immunosuppression agents are administered to the subject at least 2 weeks, 4 weeks, 6 weeks, 8 weeks, 10 weeks, 12 weeks, 16 weeks, or more, after administration of the dose of engineered hypoimmunogenic islets. In some of any embodiments, the one or more immunosuppression agents are administered to the subject at least 1 month, 2 months, 3 months, 4 months, 5 months, 6 months, or more, after administration of the dose of engineered hypoimmunogenic islets. In some of any embodiments, the one or more immunosuppression agents are administered to the subject on the same day as the dose of engineered hypoimmunogenic islets. In some of any embodiments, the one or more immunosuppression agents are administered to the subject concurrently with the dose of engineered hypoimmunogenic islets. In some of any embodiments, the one or more immunosuppression agents are administered to the subject only at least 1, 2, 3, 4, 5, 6, 7, 8, 9, 10, 11, 12, 13, or 14 days prior to administration of a first and/or second administration of the dose of engineered hypoimmunogenic islets. In some of any embodiments, the one or more immunosuppression agents are administered to the subject only at least 1 week, 2 weeks, 3 weeks, 4 weeks, 5 weeks, 6 weeks, 7 weeks, 8 weeks, 9 weeks, 10 weeks or more prior to administration of a first and/or second administration of the dose of engineered hypoimmunogenic islets. In some of any embodiments, the one or more immunosuppression agents are administered to the subject only at least 1, 2, 3, 4, 5, 6, 7, 8, 9, 10, 11, 12, 13, or 14 days after administration of a first and/or second administration of the dose of engineered hypoimmunogenic islets. In some of any embodiments, the one or more immunosuppression agents are administered to the subject only at least 1 week, 2 weeks, 3 weeks, 4 weeks, 5 weeks, 6 weeks, 7 weeks, 8 weeks, 9 weeks, or 10 weeks after administration of a first and/or second administration of the dose of engineered hypoimmunogenic islets. In some of any embodiments, the one or more immunosuppression agents are administered to the subject only at least 2 weeks, 4 weeks, 6 weeks, 8 weeks, 10 weeks, 12 weeks or 16 weeks after administration of the dose of engineered hypoimmunogenic islets. In some of any embodiments, the one or more immunosuppression agents are administered to the subject only at least 1 month, 2 months, 3 months, 4 months, 5 months or 6 months after administration of the dose of engineered hypoimmunogenic islets.

In some of any embodiments, the one or more immunosuppression agents are administered to the subject prior to the administration of the dose of engineered hypoimmunogenic islets, and continued to be administered over the course of the subject's lifespan. In some of any embodiments, the one or more immunosuppression agents are administered to the subject on the same day and/or concurrent with the administration of the dose of engineered hypoimmunogenic islets, and continued to be administered over the course of the subject's lifespan. In some of any embodiments, the one or more immunosuppression agents are administered to the subject after the administration of the dose of engineered hypoimmunogenic islets, and continued to be administered over the course of the subject's lifespan. In some of any embodiments, the one or more immunosuppression agents are administered to the subject prior to each round of the administration the dose of engineered hypoimmunogenic islets, and optionally continued to be administered over the course of the subject's lifespan. In some of any embodiments, the one or more immunosuppression agents are administered to the subject on the same day and/or concurrent with each round the administration of the dose of engineered hypoimmunogenic islets, and optionally continued to be administered over the course of the subject's lifespan. In some of any embodiments, the one or more immunosuppression agents are administered to the subject after each round of the administration of the dose of engineered hypoimmunogenic islets, and optionally continued to be administered over the course of the subject's lifespan.

In some of any embodiments, the one or more immunosuppression agents are administered to the subject at a lower dosage compared to the dosage of one or more immunosuppressive agents administered the subject to reduce immune rejection of immunogenic cells that do not comprise the modifications of the dose of engineered hypoimmunogenic islets. In some of any embodiments, the one or more immunosuppression agents comprise a small molecule or a biological product. In some of any embodiments, the biological product is a protein and/or an antibody. In some of any embodiments, the small molecule is a chemical compound or a nucleic acid. In some of any embodiments, the one or more immunosuppression agents comprise one or more immunomodulatory agents. In some of any embodiments, the one or more immunomodulatory agents are a small molecule or a biological product. In some of any embodiments, the biological product is a protein or peptide thereof and/or an antibody. In some of any embodiments, the small molecule is a chemical compound or a nucleic acid. In some of any embodiments, the one or more immunosuppression agents are a pharmaceutical salt thereof, a preform thereof and/or a derivative thereof. In some of any embodiments, the one or more immunomodulatory agents are a pharmaceutical salt thereof, a preform thereof and/or a derivative thereof.

In some of any embodiments, the one or more immunosuppression agents are selected from the group consisting of calcineurin inhibitors, steroids, alkylating agents, antibiotics, analgesics, anti-inflammatory agents, antihistamines, antiviral agents, anti-fungal agents, anti-coagulation agents, DNA synthesis inhibitors, anti-coagulation agents, hemorheologic agents, inosine monophosphate dehydrogenase (IMDH) inhibitors, Janus kinase inhibitors, mTOR inhibitors, TNF inhibitors, and anti-CD25 inhibitors In some of any embodiments, the one or more immunosuppression agents are selected from the group consisting of antithymocyte globulin (ATG), corticosteroids, prednisone, cortisone, prednisolone methylprednisolone, dexamethasone, betamethasone, hydrocortisone, methotrexate, acetaminophen, diphenhydramine, sirolimus (rapamycin), tacrolimus (FK-506), mycophenolic acid (MPA), mycophenolate mofetil (MMF), mycophenolate sodium, cyclosporine, etanercept (TNFR-Fc), azathioprine, gold salts, sulfasalazine, antimalarials, brequinar, leflunomide, mizoribine, 15-deoxyspergualine, 6-mercaptopurine, cyclophosphamide, OKT3, anti-thymocyte globulin, thymopentin (thymosin-α), fludarabine, cyclophosphamide, and an immunosuppressive antibody.

In some of any embodiments, the one or more immunosuppression agents comprise antithymocyte globulin (ATG). In some of any embodiments, at least one regimen of ATG is administered to the subject prior to, concurrent with, and/or after the administration of the dose of engineered hypoimmunogenic islets to the subject. In some of any embodiments, the at least one regimen of ATG is administered to the subject prior to the administration of the dose of engineered hypoimmunogenic islets to the subject. In some of any embodiments, the at least one regimen of ATG is administered to the subject prior to each administration of the dose of engineered hypoimmunogenic islets to the subject. In some of any embodiments, the at least one regimen of ATG is administered to the subject about 7 days prior to, about 6 days prior to, about 5 days prior to, about 4 days prior to, about 3 days prior to, about 2 days prior to, about 1 day prior to, about 12 hours prior to, about 10 hours prior to, about 8 hours prior to, about 6 hours prior to, about 4 hours prior to, about 2 hours prior to, or about 1 hour prior to the administration of the dose of engineered hypoimmunogenic islets to the subject. In some of any embodiments, the at least one regimen of ATG is administered to the subject about 2 days prior and/or about 1 day prior to the administration of the dose of engineered hypoimmunogenic islets to the subject. In some of any embodiments, the at least one regimen of ATG is administered to the subject on the same day and/or concurrent with the administration of the dose of engineered hypoimmunogenic islets to the subject. In some of any embodiments, the at least one regimen of ATG is administered to the subject on the same day and/or concurrent with each administration of the dose of engineered hypoimmunogenic islets to the subject. In some of any embodiments, a first regimen or a second regimen of ATG is administered to the subject on the same day and/or concurrent with the administration of the dose of engineered hypoimmunogenic islets to the subject. In some of any embodiments, at least one regimen of ATG is administered to the subject after the administration of the dose of engineered hypoimmunogenic islets to the subject. In some of any embodiments, a regimen of ATG is administered to the subject after each administration of the dose of engineered hypoimmunogenic islets to the subject. In some of any embodiments, the at least one regimen of ATG is administered to the subject about 1 hour after, about 2 hours after, about 4 hours after, about 6 hours after, about 8 hours after, about 10 hours after, about 12 hours, about 24 hours subsequent, or about 48 hours after the administration of the dose of engineered hypoimmunogenic islets to the subject. In some of any embodiments, the at least one regimen of ATG is administered to the subject about 48 hours after the administration of the dose of engineered hypoimmunogenic islets to the subject. In some of any embodiments, the at least one regimen of ATG is administered to the subject: i) about 2 days prior; ii) about 1 day prior; iii) on the same day; iv) about 1 day subsequent; and/or, v) about 2 days after the administration of the dose of engineered hypoimmunogenic islets to the subject. In some of any embodiments, the at least one or at least two regimens of ATG comprise between about 0.1 mg/kg and about 2.0 mg/kg ATG is administered to the subject. In some of any embodiments, the ATG regimen is administered at a lower dose. In some of any embodiments, the method comprises a regimen wherein: i) the at least one or at least two regimens of ATG comprise a dose of about 0.5 mg/kg of ATG is administered to the subject about 2 days prior to the administration of the dose of engineered hypoimmunogenic islets to the subject; ii) the at least one or two regimens of ATG comprise a dose of about 1.0 mg/kg of ATG is administered to the subject about 1 day prior to the administration of the dose of engineered hypoimmunogenic islets to the subject; and/or, iii) the at least one or at least two regimens of ATG comprise a dose of about 1.5 mg/kg of ATG is administered to the subject on the same day as the administration of the dose of engineered hypoimmunogenic islets to the subject, about 1 day after the administration of the dose of engineered hypoimmunogenic islets to the subject, and about 2 days after the administration of the dose of engineered hypoimmunogenic islets to the subject. In some of any embodiments, the ATG regimen is administered at a lower dose.

In some of any embodiments, the one or more immunosuppression agents comprise a corticosteroid. In some of any embodiments, the one or more immunosuppression agents comprise prednisone, cortisone, prednisolone methylprednisolone, dexamethasone, betamethasone, or hydrocortisone. In some of any embodiments, the one or more immunosuppression agents comprise methylprednisolone. In some of any embodiments, at least one regimen of methylprednisolone is administered to the subject prior to the administration of the dose of engineered hypoimmunogenic islets to the subject. In some of any embodiments, the at least one regimen of methylprednisolone is administered to the subject about 7 days prior to, about 6 days prior to, about 5 days prior to, about 4 days prior to, about 3 days prior to, about 2 days prior to, about 1 day prior to, about 12 hours prior to, about 10 hours prior to, about 8 hours prior to, about 6 hours prior to, about 4 hours prior to, about 2 hours prior to, or about 1 hour prior to the administration of the dose of engineered hypoimmunogenic islets to the subject. In some of any embodiments, the at least one regimen methylprednisolone is administered to the subject about 2 days prior to the administration of the dose of engineered hypoimmunogenic islets to the subject. In some of any embodiments, the at least one regimen of methylprednisolone is administered to the subject prior to the administration of a first regimen of ATG to the subject, wherein both the regimen of methylprednisolone and the first regimen of ATG are administered to the subject prior to the administration of the dose of engineered hypoimmunogenic islets to the subject. In some of any embodiments, the at least one regimen of methylprednisolone is administered to the subject about 1 hour prior to the administration of a first regimen of ATG to the subject. In some of any embodiments, the at least one regimen methylprednisolone is administered to the subject about midway through the administration of a first regimen of ATG to the subject. In some of any embodiments, the at least one regimen of methylprednisolone comprises a dose of between about 0.1 mg/kg and about 2.0 mg/kg. In some of any embodiments, the methylprednisolone regimen is administered at a lower dose. In some of any embodiments, the at least one regimen of methylprednisolone comprises about 1.0 mg/kg of methylprednisolone. In some of any embodiments, the methylprednisolone regimen is administered at a lower dose. In some of any embodiments, the methylprednisolone is administered to the subject intravenously. In some of any embodiments, the method comprises a regimen wherein: i) the at least one regimen of methylprednisolone comprises about 1.0 mg/kg of methylprednisolone administered to the subject about 1 hour prior to the administration of a first regimen ATG to the subject; and/or ii) the at least one regimen of methylprednisolone comprises about 1.0 mg/kg of methylprednisolone administered to the subject about midway through the administration of the first regimen ATG to the subject In some of any embodiments, the methylprednisolone regimen and/or the ATG regimen is administered at a lower dose.

In some of any embodiments, the one or more immunosuppression agents comprise an analgesic. In some of any embodiments, the analgesic is acetaminophen, an opioid, or a non-steroidal anti-inflammatory drug (NSAID). In some of any embodiments, at least one regimen of acetaminophen is administered to the subject prior to the administration of the dose of engineered hypoimmunogenic islets to the subject. In some of any embodiments, the at least one regimen of acetaminophen is administered to the subject about 7 days prior to, about 6 days prior to, about 5 days prior to, about 4 days prior to, about 3 days prior to, about 2 days prior to, about 1 day prior to, about 12 hours prior to, about 10 hours prior to, about 8 hours prior to, about 6 hours prior to, about 4 hours prior to, about 2 hours prior to, or about 1 hour prior to the administration of the dose of engineered hypoimmunogenic islets to the subject. In some of any embodiments, the at least one regimen of acetaminophen is administered to the subject about 2 days prior to the administration of the dose of engineered hypoimmunogenic islets to the subject. In some of any embodiments, the at least one regimen of acetaminophen is administered to the subject prior to the administration of a first regimen of ATG to the subject, wherein both the regimen of acetaminophen and the first regimen of ATG are administered to the subject prior to the administration of the dose of engineered hypoimmunogenic islets to the subject. In some of any embodiments, the at least one regimen of acetaminophen is administered to the subject about 30 minutes prior to the administration of a first regimen of ATG to the subject. In some of any embodiments, the at least one regimen of acetaminophen is administered to the subject about midway through the administration of a first regimen of ATG to the subject. In some of any embodiments, the at least one regimen of acetaminophen comprises between about 100 mg and about 1,000 mg of acetaminophen is administered to the subject. In some of any embodiments, the acetaminophen regimen is administered at a lower dose. In some of any embodiments, the at least one regimen of about 650 mg of acetaminophen is administered to the subject. In some of any embodiments, the acetaminophen regimen is administered at a lower dose. In some of any embodiments, the acetaminophen is administered to the subject orally or rectally. In some of any embodiments, the methods comprise a regimen, wherein: i) the at least one regimen of about 650 mg of acetaminophen administered to the subject about 30 minutes prior to the administration of a first regimen ATG to the subject; and/or ii) the at least one regimen of about 650 mg of acetaminophen administered to the subject about midway through the administration of the first regimen ATG to the subject In some of any embodiments, the acetaminophen regimen and/or the ATG regimen is administered at a lower dose.

In some of any embodiments, the one or more immunosuppression agents comprise an antihistamine. In some of any embodiments, the antihistamine is diphenhydramine. In some of any embodiments, at least one regimen of diphenhydramine is administered to the subject prior to the administration of the dose of engineered hypoimmunogenic islets to the subject. In some of any embodiments, the at least one regimen of diphenhydramine is administered to the subject about 7 days prior to, about 6 days prior to, about 5 days prior to, about 4 days prior to, about 3 days prior to, about 2 days prior to, about 1 day prior to, about 12 hours prior to, about 10 hours prior to, about 8 hours prior to, about 6 hours prior to, about 4 hours prior to, about 2 hours prior to, or about 1 hour prior to the administration of the dose of engineered hypoimmunogenic islets to the subject. In some of any embodiments, the at least one regimen of diphenhydramine is administered to the subject about 2 days prior to the administration of the dose of engineered hypoimmunogenic islets to the subject. In some of any embodiments, the at least one regimen of diphenhydramine is administered to the subject prior to the administration of a first regimen of ATG to the subject, wherein both the regimen of diphenhydramine and the first regimen of ATG are administered to the subject prior to the administration of a dose of engineered hypoimmunogenic islets to the subject. In some of any embodiments, the at least one regimen of diphenhydramine is administered to the subject about 30 minutes prior to the administration of a first regimen of ATG to the subject. In some of any embodiments, the at least one regimen of diphenhydramine is administered to the subject about midway through the administration of a first regimen of ATG to the subject. In some of any embodiments, the at least one regimen diphenhydramine comprises of between about 10 mg and about 100 mg of diphenhydramine is administered to the subject. In some of any embodiments, the diphenhydramine regimen is administered at a lower dose. In some of any embodiments, the at least one regimen of about 50 mg of diphenhydramine is administered to the subject. In some of any embodiments, the diphenhydramine regimen is administered at a lower dose. In some of any embodiments, the diphenhydramine is administered to the subject orally or rectally. In some of any embodiments, the method comprises a regimen wherein: i) at least one regimen of diphenhydramine comprises about 50 mg of a diphenhydramine administered to the subject about 30 minutes prior to the administration of a first regimen ATG to the subject; and/or ii) at least one regimen of diphenhydramine comprises about 50 mg of diphenhydramine is administered to the subject about midway through the administration of the first regimen ATG to the subject; In some of any embodiments, the diphenhydramine regimen and/or the ATG regimen is administered at a lower dose.

In some of any embodiments, the one or more immunosuppression agents comprise an anti-inflammatory agent. In some of any embodiments, the anti-inflammatory agent is a TNF inhibitor. In some of any embodiments, the TNF inhibitor is selected from the group consisting of infliximab, adalimumab, etanercept, golimumab, and certolizumab. In some of any embodiments, the TNF inhibitor is etanercept (TNFR-Fc). In some of any embodiments, at least one regimen of etanercept is administered to the subject prior, concurrent with, and/or after the administration of the dose of engineered hypoimmunogenic islets to the subject. In some of any embodiments, the at least one regimen of etanercept is administered to the subject prior to the administration of the dose of engineered hypoimmunogenic islets to the subject. In some of any embodiments, the at least one regimen of etanercept is administered to the subject about 7 days prior to, about 6 days prior to, about 5 days prior to, about 4 days prior to, about 3 days prior to, about 2 days prior to, about 1 day prior to, about 12 hours prior to, about 10 hours prior to, about 8 hours prior to, about 6 hours prior to, about 4 hours prior to, about 2 hours prior to, or about 1 hour prior to the administration of the dose of engineered hypoimmunogenic islets to the subject. In some of any embodiments, the at least one regimen of etanercept is administered to the subject on the same day and/or concurrent with the administration of the dose of engineered hypoimmunogenic islets to the subject. In some of any embodiments, a first regimen of etanercept is administered to the subject on the same day and/or concurrent with the administration of the dose of engineered hypoimmunogenic islets to the subject. In some of any embodiments, at least one regimen of etanercept is administered to the subject after the administration of the dose of engineered hypoimmunogenic islets to the subject. In some of any embodiments, the at least one regimen of etanercept is administered to the subject about 1 hour after, about 5 hours after, about 10 hours after, about 24 hours after, about 2 days after, about 3 days after, about 5 days, about 7 days subsequent, or about 10 days after the administration of the dose of engineered hypoimmunogenic islets to the subject. In some of any embodiments, the at least one regimen of etanercept is administered to the subject about 3 days, about 7 days, and/or about 10 days after the administration of the dose of engineered hypoimmunogenic islets to the subject. In some of any embodiments, the at least one regimen of etanercept is administered to the subject: i) on the same day; ii) about 3 days subsequent; iii) about 7 days subsequent; and/or iv) about 10 days subsequent, to the administration of the dose of engineered hypoimmunogenic islets to the subject. In some of any embodiments, the at least one regimen of etanercept comprises between about 10 mg and about 100 mg of etanercept. In some of any embodiments, the etanercept regimen is administered at a lower dose. In some of any embodiments, the at least one regimen of etanercept comprises about 50 mg of etanercept. In some of any embodiments, the etanercept regimen is administered at a lower dose. In some of any embodiments, the at least one regimen of etanercept comprises about 25 mg of etanercept. In some of any embodiments, the etanercept regimen is administered at a lower dose. In some of any embodiments, the etanercept is administered to the subject intravenously and/or subcutaneously. In some of any embodiments, the methods comprise a method wherein: i) the at least one regimen of about 50 mg of etanercept administered to the subject on the same day as the administration of the dose of engineered hypoimmunogenic islets to the subject; and/or, ii) the at least one regimen of about 25 mg etanercept administered to the subject about 3 days, about 7 days, and/or about 10 days after the administration of the dose of engineered hypoimmunogenic islets to the subject. In some of any embodiments, the etanercept regimen is administered at a lower dose. In some of any embodiments, the subject is administered at least one regimen of etanercept and at least one regimen of ATG. In some of any embodiments, the subject is administered the at least one regimen of ATG prior to the at least one regimen of etanercept. In some of any embodiments, i) the at least one regimen of ATG comprises about 40 mg/kg of ATG mg administered to the subject each day for four consecutive days; ii) the at least one regimen of etanercept comprises about 25 mg of etanercept administered to the subject twice a week for two consecutive weeks after i); and iii) the at least one regimen of etanercept comprises about 25 mg of etanercept administered to the subject once a month for about four months after ii). In some of any embodiments, the at least one etanercept regimen and/or the at least one ATG regimen is administered at a lower dose. In some of any embodiments, the subject is administered at least one regimen of etanercept and at least one regimen of an IL-1 receptor antagonist.

In some of any embodiments, the one or more immunosuppression agents comprise an mTOR inhibitor. In some of any embodiments, the mTOR inhibitor is sirolimus (rapamycin). In some of any embodiments, at least one regimen of sirolimus is administered to the subject prior, concurrent with, and/or after the administration of the dose of engineered hypoimmunogenic islets to the subject. In some of any embodiments, at least one regimen of sirolimus is administered to the subject prior to the administration of the dose of engineered hypoimmunogenic islets to the subject.

In some of any embodiments, the at least one regimen of sirolimus is administered to the subject about 7 days prior to, about 6 days prior to, about 5 days prior to, about 4 days prior to, about 3 days prior to, about 2 days prior to, about 1 day prior to, about 12 hours prior to, about 10 hours prior to, about 8 hours prior to, about 6 hours prior to, about 4 hours prior to, about 2 hours prior to, or about 1 hour prior to the administration of the dose of engineered hypoimmunogenic islets to the subject.

In some of any embodiments, at least one regimen of sirolimus is administered to the subject on the same day and/or concurrent with the administration of the dose of engineered hypoimmunogenic islets to the subject. In some of any embodiments, at least one regimen of sirolimus is administered to the subject after the administration of the dose of engineered hypoimmunogenic islets to the subject. In some of any embodiments, the at least one regimen of sirolimus is administered to the subject about 1 hour after, about 5 hours after, about 10 hours after, or about 24 hours after, about 3 months after, about 6 months after, about 12 months after, about 24 months after, about 36 months after, about 48 months after, about 60 months after, or more, the administration of the dose of engineered hypoimmunogenic islets to the subject. In some of any embodiments, a total daily dosage of sirolimus administered to the subject yields a blood trough level of between about 1 ng/mL and about 30 ng/mL, between about 2 ng/mL and about 25 ng/mL, between about 5 ng/mL and about 20 ng/mL, or between about 10 ng/mL and about 15 ng/mL, inclusive of each. In some of any embodiments, a regimen of between about 0.1 mg/kg and about 0.2 mg/kg of sirolimus is administered to the subject. In some of any embodiments, the sirolimus regimen is administered at a lower dose. In some of any embodiments, the sirolimus is administered to the subject orally.

In some of any embodiments, i) a regimen of about 0.2 mg/kg of sirolimus is administered to the subject on the same day as the administration of the dose of engineered hypoimmunogenic islets to the subject; ii) a regimen of about 0/1 mg/kg of sirolimus is administered to the subject each day up to about 3 months after the administration of the dose of engineered hypoimmunogenic islets to the subject, wherein the total daily dosage of sirolimus administered to the subject yields a blood trough level of between about 12 ng/mL and about 15 ng/mL for about 3 months after the administration of the composition and between about 7 ng/mL and about 10 ng/mL thereafter. In some of any embodiments, the sirolimus regimen is administered at a lower dose.

In some of any embodiments, the one or more immunosuppression agents comprise a calcineurin inhibitor. In some of any embodiments, the calcineurin inhibitor is tacrolimus (FK-506). In some of any embodiments, at least one regimen of tacrolimus is administered to the subject prior, concurrent with, and/or after the administration of the dose of engineered hypoimmunogenic islets to the subject.

In some of any embodiments, at least one regimen of tacrolimus is administered to the subject prior to the administration of the dose of engineered hypoimmunogenic islets to the subject. In some of any embodiments, the at least one regimen of tacrolimus is administered to the subject about 7 days prior to, about 6 days prior to, about 5 days prior to, about 4 days prior to, about 3 days prior to, about 2 days prior to, about 1 day prior to, about 12 hours prior to, about 10 hours prior to, about 8 hours prior to, about 6 hours prior to, about 4 hours prior to, about 2 hours prior to, or about 1 hour prior to the administration of the dose of engineered hypoimmunogenic islets to the subject. In some of any embodiments, at least one regimen of tacrolimus is administered to the subject on the same day and/or concurrent with the administration of the dose of engineered hypoimmunogenic islets to the subject. In some of any embodiments, a first regimen of tacrolimus is administered to the subject on the same day and/or concurrent with the administration of the dose of engineered hypoimmunogenic islets to the subject. In some of any embodiments, at least one regimen of tacrolimus is administered to the subject after the administration of the dose of engineered hypoimmunogenic islets to the subject. In some of any embodiments, the at least one regimen of tacrolimus is administered to the subject about 1 hour after, about 5 hours after, about 10 hours after, or about 24 hours after, about 3 months after, about 6 months after, about 12 months after, about 24 months after, about 36 months after, about 48 months after, about 60 months after, or more, the administration of the dose of engineered hypoimmunogenic islets to the subject. In some of any embodiments, the total daily dosage of tacrolimus administered to the subject yields a blood trough level of between about 1 ng/mL and about 30 ng/mL, between about 2 ng/mL and about 25 ng/mL, between about 5 ng/mL and about 20 ng/mL, or between about 10 ng/mL and about 15 ng/mL, inclusive of each. In some of any embodiments, the total daily dosage of tacrolimus administered to the subject yields a blood trough level of between about 5 ng/mL and about 10 ng/mL, inclusive of each. In some of any embodiments, the total daily dosage of tacrolimus administered to the subject yields a blood trough level of between about 10 ng/mL and about 15 ng/mL, inclusive of each.

In some of any embodiments, a regimen of between about 0.1 mg and about 5 mg of tacrolimus is administered to the subject. In some of any embodiments, the tacrolimus regimen is administered at a lower dose. In some of any embodiments, the one or more immunosuppression agents comprise an inosine-”-monophosphate dehydrogenase (IMPDH) inhibitor. In some of any embodiments, the IMPDH inhibitor is MPA, MMF, or MS. In some of any embodiments, the IMPDH inhibitor is mycophenolic acid (MPA). In some of any embodiments, at least one regimen of MPA is administered to the subject prior, concurrent with, and/or after the administration of the dose of engineered hypoimmunogenic islets to the subject. In some of any embodiments, at least one regimen of MPA is administered to the subject prior to the administration of the dose of engineered hypoimmunogenic islets to the subject. In some of any embodiments, the at least one regimen of MPA is administered to the subject about 7 days prior to, about 6 days prior to, about 5 days prior to, about 4 days prior to, about 3 days prior to, about 2 days prior to, about 1 day prior to, about 12 hours prior to, about 10 hours prior to, about 8 hours prior to, about 6 hours prior to, about 4 hours prior to, about 2 hours prior to, or about 1 hour prior to the administration of the dose of engineered hypoimmunogenic islets to the subject. In some of any embodiments, at least one regimen of MPA is administered to the subject on the same day and/or concurrent with the administration of the dose of engineered hypoimmunogenic islets to the subject. In some of any embodiments, a first regimen of MPA is administered to the subject on the same day and/or concurrent with the administration of the dose of engineered hypoimmunogenic islets to the subject. In some of any embodiments, at least one regimen of MPA is administered to the subject after the administration of the dose of engineered hypoimmunogenic islets to the subject. In some of any embodiments, the at least one regimen of MPA is administered to the subject about 1 hour after, about 5 hours after, about 10 hours after, or about 24 hours after, about 3 months after, about 6 months after, about 12 months after, about 24 months after, about 36 months after, about 48 months after, about 60 months after, or more, the administration of the dose of engineered hypoimmunogenic islets to the subject.

In some of any embodiments, the MPA is mycophenolate mofetil (MMF). In some of any embodiments, the total daily dosage of MMF is between about 10 mg and about 3000 mg, about 500 mg and about 3000 mg, between about 1000 mg and about 2500 mg, or between about 1500 mg and about 2000 mg, inclusive of each. In some of any embodiments, the total daily dosage of MMF is about 100 mg, 500 mg, 1000 mg, about 1500 mg, about 2000 mg, or about 2500 mg. In some of any embodiments, the total daily dosage of MMF is lower.

In some of any embodiments, the MPA is mycophenolate sodium (MS). In some of any embodiments, the total daily dosage of MS is between about 10 mg and about 2700 mg, about 360 mg and about 2700 mg, between about 720 mg and about 2160 mg, or between about 720 mg and about 1620 mg, inclusive of each. In some of any embodiments, the total daily dosage of MS is about 100 mg, about 360 mg, about 720 mg, about 1080 mg, or about 1440 mg. In some of any embodiments, the total daily dosage of MS is lower.

In some of any embodiments, the subject is administered at least one regimen of tacrolimus and at least one regimen of MPA.

In some of any embodiments, the one or more immunosuppression agents comprise cyclosporine. In some of any embodiments, at least one regimen of cyclosporine is administered to the subject prior, concurrent with, and/or after the administration of the dose of engineered hypoimmunogenic islets to the subject. In some of any embodiments, at least one regimen of cyclosporine is administered to the subject when the subject displays intolerance to a regimen of tacrolimus. In some of any embodiments, at least one regimen of cyclosporine is administered to the subject on the same day and/or concurrent with the administration of the dose of engineered hypoimmunogenic islets to the subject. In some of any embodiments, a first regimen of cyclosporine is administered to the subject on the same day and/or concurrent with the administration of the dose of engineered hypoimmunogenic islets to the subject. In some of any embodiments, at least one regimen of cyclosporine is administered to the subject after the administration of the dose of engineered hypoimmunogenic islets to the subject. In some of any embodiments, the at least one regimen of cyclosporine is administered to the subject about 1 hour after, about 5 hours after, about 10 hours after, or about 24 hours after, about 3 months after, about 6 months after, about 12 months after, about 24 months after, about 36 months after, about 48 months after, about 60 months after, or more, the administration of the dose of engineered hypoimmunogenic islets to the subject. In some of any embodiments, the total daily dosage of cyclosporine administered to the subject yields a blood trough level of between about 50 ng/mL and about 300 ng/mL, between about 100 ng/mL and about 250 ng/mL, between about 200 ng/mL and about 300 ng/mL, or between about 150 ng/mL and about 200 ng/mL, inclusive of each.

In some of any embodiments, a regimen of between about 2 mg/kg and about 10 mg/kg of cyclosporine is administered to the subject each day. In some of any embodiments, the cyclosporine regimen is administered at a lower dose. In some of any embodiments, a regimen of about 6 mg/kg of cyclosporine is administered to the subject each day. In some of any embodiments, the cyclosporine regimen is administered at a lower dose.

In some of any embodiments, the subject is administered at least one regimen of cyclosporine and at least one regimen of MPA. In some of any embodiments, the subject is administered at least one regimen of cyclosporine and at least one regimen of ATG. In some of any embodiments, the subject is administered the at least one regimen of ATG prior to the at least one regimen of cyclosporine. In some of any embodiments, wherein: i) a regimen of about 40 mg/kg of ATG mg is administered to the subject each day for four consecutive days; and ii) a regimen of between about 10 mg/kg and about 12 mg/kg of cyclosporine is administered to the subject each day for six months after i). In some of any embodiments, the cyclosporine regimen and/or the ATG regimen is administered at a lower dose.

In some of any embodiments, the one or more immunosuppression agents comprise an antibody for binding to MHC, CD2, CD3, CD4, CD7, CD28, B7, CD25, CD40, CD45, CD95, IFN-gamma, TNF-alpha, IL-2Ralpha, IL-4, IL-5, IL-6R, IL-6, IGF, IGFR1, IL-7, IL-8, IL-10, CD11alpha, or CD58, and antibodies binding to any of their ligands. In some of any embodiments, the one or more immunosuppression agents comprise soluble IL-15R, IL-10, B7 molecules such as B7-1, B7-2, variants thereof, and fragments thereof, ICOS, and OX40. In some of any embodiments, the one or more immunosuppression agents comprise an inhibitor of a negative T cell regulator, such as an antibody against CTLA-4, or similar agents. In some of any embodiments, the one or more immunosuppression agents comprise an anti-CD25 antibody or an anti-IL-2Ralpha antibody. In some of any embodiments, the anti-CD25 antibody or the anti-IL-2Ralpha antibody is selected from the group consisting of basiliximab, daclizumab, and alemtuzumab.

In some of any embodiments, the one or more immunosuppression agents comprise basiliximab. In some of any embodiments, at least one regimen of basiliximab is administered to the subject on the same day, concurrent with, and/or after the administration of the dose of engineered hypoimmunogenic islets to the subject. In some of any embodiments, the at least one regimen of basiliximab is administered to the subject about 1 hour after, about 5 hours after, about 10 hours after, or about 24 hours after, about 3 months after, about 6 months after, about 12 months after, about 24 months after, about 36 months after, about 48 months after, about 60 months after, or more, the administration of the dose of engineered hypoimmunogenic islets to the subject. In some of any embodiments, at least one regimen of basiliximab is administered to the subject after the administration of at least one regimen of ATG to the subject. In some of any embodiments, at least one of basiliximab is administered to the subject after the administration of at least one regimen of ATG and after the administration of the dose of engineered hypoimmunogenic islets to the subject. In some of any embodiments, at least one regimen of basiliximab is administered to the subject about 4 days after the administration of the dose of engineered hypoimmunogenic islets to the subject. In some of any embodiments, a regimen of between about 10 mg and about 30 mg of basiliximab is administered to the subject. In some of any embodiments, a regimen of between about 20 mg of basiliximab is administered to the subject. In some of any embodiments, the basiliximab regimen is administered at a lower dose. In some of any embodiments, wherein; i) a regimen of about 20 mg of basiliximab is administered to the subject on the same day as the administration of the dose of engineered hypoimmunogenic islets to the subject; and/or, ii) a regimen of about 20 mg of basiliximab is administered to the subject about 4 days after the administration of the dose of engineered hypoimmunogenic islets to the subject.

In some of any embodiments, the one or more immunosuppression agents comprise daclizumab. In some of any embodiments, at least one regimen of daclizumab is administered to the subject on the same day, concurrent with, and/or after the administration of the dose of engineered hypoimmunogenic islets to the subject. In some of any embodiments, the at least one regimen of daclizumab is administered to the subject about 1 hour after, about 5 hours after, about 10 hours after, or about 24 hours after, about 3 months after, about 6 months after, about 12 months after, about 24 months after, about 36 months after, about 48 months after, about 60 months after, or more, the administration of the dose of engineered hypoimmunogenic islets to the subject. In some of any embodiments, the at least one regimen of daclizumab is administered to the subject about every 14 days after the administration of the dose of engineered hypoimmunogenic islets to the subject. In some of any embodiments, a regimen of between about 0.5 mg/kg and about 2 mg/kg of daclizumab is administered to the subject. In some of any embodiments, the daclizumab regimen is administered at a lower dose. In some of any embodiments, a regimen of about 1 mg/kg of daclizumab is administered to the subject. In some of any embodiments, the daclizumab regimen is administered at a lower dose.

In some of any embodiments, the subject is administered at least one regimen of tacrolimus and at least one regimen of sirolimus. In some of any embodiments, the subject is administered at least one regimen of tacrolimus and at least one regimen of daclizumab. In some of any embodiments, the subject is administered at least one regimen of sirolimus and at least one regimen of daclizumab. In some of any embodiments, the subject is administered at least one regimen of tacrolimus, at least one regimen of sirolimus, and at least one regimen of daclizumab.

In some of any embodiments, wherein: i) a regimen of about 0.2 mg/kg of sirolimus is administered to the subject on the same day as the administration of the dose of engineered hypoimmunogenic islets to the subject; ii) a regimen of about 0.1 mg/kg of sirolimus is administered to the subject each day after the administration of the dose of engineered hypoimmunogenic islets to the subject, wherein the total daily dosage of sirolimus administered to the subject yields a blood trough level of between about 12 ng/mL and about 15 ng/mL, inclusive of each, for the first three months after the administration of the composition to the subject, and wherein the total daily dosage of sirolimus administered to the subject yields a blood trough level of between about 7 ng/mL and about 10 ng/mL, inclusive of each, after the first three months; iii) a regimen of about 1 mg of tacrolimus is administered to the subject on the same day as the administration of the dose of engineered hypoimmunogenic islets to the subject; iv) a regimen of about 1 mg of tacrolimus is administered to the subject twice a day about 12 hours after the administration of the dose of engineered hypoimmunogenic islets to the subject, wherein the total daily dosage of tacrolimus administered to the subject yields a blood trough level of between about 3 ng/mL and about 6 ng/mL, inclusive of each; and/or v) a regimen of about 1 mg/kg of daclizumab is administered to the subject about every 14 days after the administration of the dose of engineered hypoimmunogenic islets to the subject.

In some of any embodiments, the sirolimus regimen, tacrolimus regimen, and or the daclizumab regimen is administered at a lower dose. In some of any embodiments, the subject is not administered glucocorticoids. In some of any embodiments, the one or more immunosuppression agents comprise alemtuzumab. In some of any embodiments, at least one regimen of alemtuzumab is administered to the subject prior to, on the same day, concurrent with, and/or after the administration of the dose of engineered hypoimmunogenic islets to the subject. In some of any embodiments, at least one regimen of alemtuzumab is administered to the subject before at least one regimen of tacrolimus and/or MPA is administered to the subject. In some of any embodiments, the at least one regimen of alemtuzumab and the at least one regimen of tacrolimus and/or MPA is administered to the subject after the administration of the dose of engineered hypoimmunogenic islets of the subject.

In some of any embodiments, the one or more immunosuppression agents comprise an anti-CD3 antibody. In some of any embodiments, the anti-CD3 antibody is an anti-CD3e antibody. In some of any embodiments, the anti-CD3 antibody is OKT3. In some of any embodiments, the one or more immunosuppression agents comprise an anti-IL-33 antibody. In some of any embodiments, the one or more immunosuppression agents comprise an anti-CD95 antibody. In some of any embodiments, the one or more immunosuppression agents comprise fingolimod hydrochloride. In some of any embodiments, the one or more immunosuppression agents comprise liposomal clodronate. In some of any embodiments, the one or more immunosuppression agents comprise CTLA4-Ig. In some of any embodiments, the one or more immunosuppression agents comprise aryl hydrocarbon receptor (AhR) ligand 2-(1′H-indole-3′-carbonyl)-thiazole-4-carboxylic acid methyl ester (ITE). In some of any embodiments, the one or more immunosuppression agents comprise T1D autoantigen proinsulin. In some of any embodiments, the one or more immunosuppression agents comprise TGF-β1. In some of any embodiments, the one or more immunosuppression agents comprise dexamethasone. In some of any embodiments, the one or more immunosuppression agents comprise methotrexate. In some of any embodiments, the one or more immunosuppression agents comprise gold salts. In some of any embodiments, the one or more immunosuppression agents comprise sulfasalazine. In some of any embodiments, the one or more immunosuppression agents comprise one or more anti-malarials. In some of any embodiments, the one or more immunosuppression agents comprise brequinar. In some of any embodiments, the one or more immunosuppression agents comprise leflunomide. In some of any embodiments, the one or more immunosuppression agents comprise mizoribine. In some of any embodiments, the one or more immunosuppression agents comprise 15-deoxyspergualine. In some of any embodiments, the one or more immunosuppression agents comprise 6-mercaptopurine. In some of any embodiments, the one or more immunosuppression agents comprise cyclophosphamide. In some of any embodiments, the one or more immunosuppression agents comprise anti-thymocyte globulin. In some of any embodiments, the one or more immunosuppression agents comprise an antibiotic agent.

In some of any embodiments, the antibiotic agent is selected from the group consisting of trimethoprim/sulfamethoxaxole, penicillin, amoxicillin, cephalexin, erythromycin (E-Mycin), clarithromycin (Biaxin), azithromycin (Zithromax), ciprofolxacin (Cipro), levofloxacin (Levaquin), ofloxacin (Floxin), co-trimoxazole (Bactrim) and trimethoprim (Proloprim), tetracycline (Sumycin, Panmycin) and doxycycline (Vibramycin), gentamicin (Garamycin), and tobramycin (Tobrex). In some of any embodiments, the antibiotic agent is trimethoprim/sulfamethoxaxole. In some of any embodiments, at least one regimen of trimethoprim/sulfamethoxaxole is administered to the subject after the administration of the dose of engineered hypoimmunogenic islets to the subject.

In some of any embodiments, the at least one regimen of trimethoprim/sulfamethoxaxole is administered to the subject about 1 hour after, about 5 hours after, about 10 hours after, or about 24 hours after, about 3 months after, about 6 months after, about 12 months after, about 24 months after, about 36 months after, about 48 months after, about 60 months after, or more, the administration of the dose of engineered hypoimmunogenic islets to the subject. In some of any embodiments, the at least one regimen of trimethoprim/sulfamethoxaxole is administered to the subject every day for about 6 months after the administration of the dose of engineered hypoimmunogenic islets to the subject. In some of any embodiments, a regimen of between about 50 mg and about 500 mg trimethoprim/sulfamethoxaxole is administered to the subject. In some of any embodiments, the trimethoprim/sulfamethoxaxole regimen is administered at a lower dose. In some of any embodiments, a regimen of between about 80 mg and about 400 mg trimethoprim/sulfamethoxaxole is administered to the subject. In some of any embodiments, the trimethoprim/sulfamethoxaxole regimen is administered at a lower dose. In some of any embodiments, the one or more immunosuppression agents comprise an antifungal agent. In some of any embodiments, the antifungal agent is selected from the group consisting of clotrimazole, miconazole, ketoconazole, itraconazole, and fluconazole. In some of any embodiments, the antifungal agent is clotrimazole. In some of any embodiments, at least one regimen of clotrimazole is administered to the subject prior to, on the same day, concurrent with, and/or after the administration of the dose of engineered hypoimmunogenic islets to the subject. In some of any embodiments, a regimen of clotrimazole is administered to the subject about four times each day. In some of any embodiments, at least one regimen of clotrimazole is administered to the subject each day up to about three months after the administration of the dose of engineered hypoimmunogenic islets to the subject

In some of any embodiments, the one or more immunosuppression agents comprise an antiviral agent. In some of any embodiments, the antiviral agent is selected from the group consisting of darunavir, atazanavir, ritonavir, acyclovir, valacyclovir, valganciclovir, tenofovir, and raltegravir. In some of any embodiments, the antiviral agent is an anti-cytomegaloviral agent. In some of any embodiments, the antiviral agent is valganciclovir. In some of any embodiments, at least one regimen of valganciclovir is administered to the subject after the administration of the dose of engineered hypoimmunogenic islets to the subject. In some of any embodiments, the at least one regimen of valganciclovir is administered to the subject about 1 hour after, about 5 hours after, about 10 hours after, or about 24 hours after, about 3 months after, about 6 months after, about 12 months after, about 24 months after, about 36 months after, about 48 months after, about 60 months after, or more, the administration of the dose of engineered hypoimmunogenic islets to the subject. In some of any embodiments, a regimen of between about 300 mg and about 1,000 mg valganciclovir is administered to the subject. In some of any embodiments, the valganciclovir regimen is administered at a lower dose. In some of any embodiments, a regimen of about 450 mg valganciclovir is administered to the subject each day after the administration of the dose of engineered hypoimmunogenic islets to the subject. In some of any embodiments, the valganciclovir regimen is administered at a lower dose. In some of any embodiments, a regimen of about 900 mg valganciclovir is administered to the subject each day after about day 12 after the administration of the dose of engineered hypoimmunogenic islets to the subject. In some of any embodiments, the valganciclovir regimen is administered at a lower dose. In some of any embodiments, the regimen of 900 mg valganciclovir is administered to the subject through about week 14 after the administration of the dose of engineered hypoimmunogenic islets to the subject.

In some of any embodiments, the one or more immunosuppression agents comprise a hemorheologic agent. In some of any embodiments, the hemorheologic agent is pentoxifylline. In some of any embodiments, at least one regimen of pentoxifylline is administered to the subject prior to, on the same day as, concurrent with, and/or after the administration of the dose of engineered hypoimmunogenic islets to the subject. In some of any embodiments, at least one regimen of pentoxifylline is administered to the subject prior to the administration of the dose of engineered hypoimmunogenic islets to the subject.

In some of any embodiments, the at least one regimen of pentoxifylline is administered to the subject about 7 days prior to, about 6 days prior to, about 5 days prior to, about 4 days prior to, about 3 days prior to, about 2 days prior to, about 1 day prior to, about 12 hours prior to, about 10 hours prior to, about 8 hours prior to, about 6 hours prior to, about 4 hours prior to, about 2 hours prior to, or about 1 hour prior to the administration of the dose of engineered hypoimmunogenic islets to the subject. In some of any embodiments, the at least one regimen of pentoxifylline is administered to the subject about 2 days prior to the administration of the dose of engineered hypoimmunogenic islets to the subject.

In some of any embodiments, at least one regimen of pentoxifylline is administered to the subject after the administration of the dose of engineered hypoimmunogenic islets to the subject.

In some of any embodiments, the at least one regimen of pentoxifylline is administered to the subject about 1 hour after, about 5 hours after, about 10 hours after, or about 24 hours after, about 3 months after, about 6 months after, about 12 months after, about 24 months after, about 36 months after, about 48 months after, about 60 months after, or more, the administration of the dose of engineered hypoimmunogenic islets to the subject. In some of any embodiments, at least one regimen of pentoxifylline is administered to the subject through about day 7 after the administration of the dose of engineered hypoimmunogenic islets to the subject.

In some of any embodiments, a regimen of between about 300 mg and about 500 mg of pentoxifylline is administered to the subject. In some of any embodiments, the pentoxifylline regimen is administered at a lower dose.

In some of any embodiments, the one or more immunosuppression agents comprise one or more anticoagulation agents. In some of any embodiments, the one or more anticoagulation agents are selected from the group consisting of aspirin, enoxaparin, and heparin. In some of any embodiments, the one or more anticoagulation agents is aspirin. In some of any embodiments, at least one regimen of aspirin is administered to the subject after the administration of the dose of engineered hypoimmunogenic islets to the subject. In some of any embodiments, the one or more anticoagulation agents is enoxaparin. In some of any embodiments, at least one regimen of enoxaparin is administered to the subject after the administration of the dose of engineered hypoimmunogenic islets to the subject. In some of any embodiments, the one or more anticoagulation agents is heparin. In some of any embodiments, at least one regimen of heparin is administered to the subject after the administration of the dose of engineered hypoimmunogenic islets to the subject. In some of any embodiments, at least one regimen of enoxaparin is administered to the subject after the administration of at least one regimen of heparin to the subject.

In some of any embodiments, the one or more immunosuppression agents comprise a DNA synthesis inhibitor. In some of any embodiments, the DNA synthesis inhibitor is fludarabine. In some of any embodiments, at least one regimen of fludarabine is administered to the subject prior to, concurrent with, and/or after the administration of the dose of engineered hypoimmunogenic islets to the subject. In some of any embodiments, at least one regimen of fludarabine is administered to the subject prior to the administration of the dose of engineered hypoimmunogenic islets to the subject.

In some of any embodiments, the at least one regimen of fludarabine is administered to the subject about 14 days prior to, about 10 days prior to, 7 days prior to, about 6 days prior to, about 5 days prior to, about 4 days prior to, about 3 days prior to, about 2 days prior to, about 1 day prior to, about 12 hours prior to, about 10 hours prior to, about 8 hours prior to, about 6 hours prior to, about 4 hours prior to, about 2 hours prior to, or about 1 hour prior to the administration of the dose of engineered hypoimmunogenic islets to the subject. In some of any embodiments, a first regimen of fludarabine is administered to the subject about 2 days to about 14 days prior to the administration of the dose of engineered hypoimmunogenic islets to the subject. In some of any embodiments, a regimen of fludarabine is administered to the subject each day for about 2 days, about 3 days, or about 4 days prior to the administration of the dose of engineered hypoimmunogenic islets to the subject. In some of any embodiments, a regimen of fludarabine is administered to the subject on day 5, day 4, and day 3 prior to the administration of the dose of engineered hypoimmunogenic islets to the subject.

In some of any embodiments, a regimen of between about 10 mg/m² and about 40 mg/m² of fludarabine is administered to the subject. In some of any embodiments, the fludarabine regimen is administered at a lower dose. In some of any embodiments, a regimen of about 30 mg/m² of fludarabine is administered to the subject. In some of any embodiments, the fludarabine regimen is administered at a lower dose. In some of any embodiments, fludarabine is administered to the subject intravenously.

In some of any embodiments, the one or more immunosuppression agents comprise an alkylating agent. In some of any embodiments, the alkylating agent is cyclophosphamide. In some of any embodiments, at least one regimen of cyclophosphamide is administered to the subject prior to, concurrent with, and/or after the administration of the dose of engineered hypoimmunogenic islets to the subject. In some of any embodiments, at least one regimen of cyclophosphamide is administered to the subject prior to the administration of the dose of engineered hypoimmunogenic islets to the subject. In some of any embodiments, the at least one regimen of cyclophosphamide is administered to the subject about 14 days prior to, about 10 days prior to, 7 days prior to, about 6 days prior to, about 5 days prior to, about 4 days prior to, about 3 days prior to, about 2 days prior to, about 1 day prior to, about 12 hours prior to, about 10 hours prior to, about 8 hours prior to, about 6 hours prior to, about 4 hours prior to, about 2 hours prior to, or about 1 hour prior to the administration of the dose of engineered hypoimmunogenic islets to the subject. In some of any embodiments, a first regimen of cyclophosphamide is administered to the subject about 2 days to about 14 days prior to the administration of the dose of engineered hypoimmunogenic islets to the subject. In some of any embodiments, a regimen of cyclophosphamide is administered to the subject each day for about 2 days, about 3 days, or about 4 days prior to the administration of the dose of engineered hypoimmunogenic islets to the subject. In some of any embodiments, a regimen of cyclophosphamide is administered to the subject on day 5, day 4, and day 3 prior to the administration of the dose of engineered hypoimmunogenic islets to the subject.

In some of any embodiments, a regimen of between about 400 mg/m² and about 600 mg/m² of cyclophosphamide is administered to the subject. In some of any embodiments, the cyclophosphamide regimen is administered at a lower dose. In some of any embodiments, a regimen of about 500 mg/m² of cyclophosphamide is administered to the subject. In some of any embodiments, the cyclophosphamide regimen is administered at a lower dose. In some of any embodiments, cyclophosphamide is administered to the subject intravenously.

In some of any embodiments, at least one regimen of fludarabine and at least one regimen of cyclophosphamide is administered to the subject. In some of any embodiments, the at least one regimen of fludarabine is administered to the subject prior to the administration of the at least one regimen of cyclophosphamide to the subject. In some of any embodiments, the at least one regimen of fludarabine and the at least one regimen of cyclophosphamide are administered to the subject prior to the administration of the dose of engineered hypoimmunogenic islets to the subject.

In some of any embodiments, wherein: i) a regimen of about 30 mg/m² of fludarabine and a regimen of about 500 mg/m² of cyclophosphamide are administered to the subject each day for 3 consecutive days about 2 days to about 7 days prior to the administration of the dose of engineered hypoimmunogenic islets to the subject; ii) a regimen of about 30 mg/m² of fludarabine and a regimen of about 500 mg/m² of cyclophosphamide are administered to the subject each day for 2 consecutive days about 2 days to about 14 days prior to the administration of the dose of engineered hypoimmunogenic islets to the subject; or, iii) a regimen of about 30 mg/m² of fludarabine and a regimen of about 500 mg/m² of cyclophosphamide are administered to the subject on day 5, day 4, and day 3 prior to the administration of the dose of engineered hypoimmunogenic islets to the subject. In some of any embodiments, the fludarabine regimen and/or the cyclophosphamide regimen is administered at a lower dose.

In some of any embodiments, further comprising tapering the administration of the one or more immunosuppression agents. In some of any embodiments, the tapering comprises gradually reducing the amount of the one or more immunosuppression agents that are administered to the subject. In some of any embodiments, the tapering is completed when the subject is not administered at least one of the one or more immunosuppression agents.

In some of any embodiments, the one or more molecules that regulate cell surface protein expression of the one or more MHC class I molecules are B2M. In some of any embodiments, the modifications comprise a modification that regulates cell surface protein expression of the one or more MHC class I molecules and the modification inactivates or disrupts one or more alleles of B2M. In some of any embodiments, the modification that inactivates or disrupts one or more alleles of B2M reduces mRNA expression of the B2M gene. In some of any embodiments, the modification that inactivates or disrupts one or more alleles of B2M reduces protein expression of B2M. In some of any embodiments, the modification that inactivates or disrupts one or more alleles of B2M comprises: inactivation or disruption of one allele of the B2M gene; inactivation or disruption of both alleles of the B2M gene; or inactivation or disruption of all B2M coding alleles in the cell. In some of any embodiments, the inactivation or disruption comprises an indel in the B2M gene. In some of any embodiments, the inactivation or disruption comprises a frameshift mutation or a deletion of a contiguous stretch of genomic DNA of the B2M gene.

In some of any embodiments, the modification is a modification that regulates expression of the one or more MHC class II molecules, and the modification inactivates or disrupts one or more alleles of CIITA. In some of any embodiments, the modification that inactivates or disrupts one or more alleles of CIITA reduces protein expression of CIITA. In some of any embodiments, the modification that inactivates or disrupts one or more alleles of CIITA comprises: inactivation or disruption of one allele of the CIITA gene; inactivation or disruption of both alleles of the CIITA gene; or inactivation or disruption of all CIITA coding alleles in the cell. In some of any embodiments, the inactivation or disruption comprises an indel in the CIITA gene. In some of any embodiments, the inactivation or disruption is a frameshift mutation or a deletion of a contiguous stretch of genomic DNA of the CIITA gene.

In some of any embodiments, the expression of HLA-A, HLA-B, HLA-C, HLA-DP, HLA-DQ, and HLA-DR are reduced in the engineered hypoimmunogenic islets.

In some of any embodiments, the one or more tolerogenic factors is selected from the group consisting of CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, and MANF.

In some of any embodiments, at least one of the one or more tolerogenic factors is CD47. In some of any embodiments, the one or more tolerogenic factors is CD47. In some embodiments, the CD47 is an engineered CD47 protein. In some embodiments, the engineered CD47 protein comprises (a) one or more extracellular domains; and (b) one or more membrane tethers; wherein the one or more extracellular domains comprise a signal-regulatory protein alpha (SIRPα) interaction motif and wherein the engineered protein does not comprise one or more full-length CD47 intracellular domains. In some embodiments, the SIRPα interaction motif is or comprises a CD47 extracellular domain or a portion thereof. In some embodiments, the SIRPα interaction motif is or comprises a SIRPα antibody or a portion thereof.

In some of any embodiments, the modification that increases expression of the one or more tolerogenic factors comprises an exogenous polynucleotide encoding the one or more tolerogenic factors. In some of any embodiments, the exogenous polynucleotide encoding the one or more tolerogenic factors is integrated into the genome of the engineered hypoimmunogenic islets. In some of any embodiments, the one or more tolerogenic factors comprises CD47 and the engineered hypoimmunogenic islets expresses CD47 at a first level that is greater than at or about 5-fold over a second level expressed by the control or wild-type islet cell. In some of any embodiments, CD47 is expressed at a first level that is greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by the control or wild-type islet cell.

In some of any embodiments, the one or more tolerogenic factors comprises CD47 and CD47 is expressed by the engineered hypoimmunogenic islets at greater than at or about 20,000 molecules per cell. In some of any embodiments, CD47 is expressed by the engineered hypoimmunogenic islets at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

In some of any embodiments, the engineered hypoimmunogenic islets has the phenotype B2Mindel/indel; CIITAindel/indel; CD47tg. In some of any embodiments, among the dose of cells engineered hypoimmunogenic islets at least 85% of the cells have the modifications. In some of any embodiments, at least 90%, at least 92%, at least 95% or at least 98% of the cells have the modifications. In some of any embodiments, among the dose of cells engineered hypoimmunogenic islets at least 85% of the cells have the phenotype has the phenotype B2Mindel/indel; CIITAindel/indel; CD47tg. In some of any embodiments, at least 90%, at least 92%, at least 95% or at least 98% of the cells have the phenotype.

In some of any embodiments, the engineered hypoimmunogenic islets exhibits one or more functions of a wild-type or control beta islet cell, optionally wherein the one or more functions is selected from the group consisting of in vitro glucose-stimulated insulin secretion (GSIS), glucose metabolism, maintaining fasting blood glucose levels, secreting insulin in response to glucose injections in vivo, and clearing glucose after a glucose injection in vivo. In some of any embodiments, the engineered hypoimmunogenic islets is capable of glucose-stimulated insulin secretion (GSIS), optionally wherein the insulin secretion is in a perfusion GSIS assay. In some of any embodiments, the GSIS is dynamic GSIS comprising first and second phase dynamic insulin secretion. In some of any embodiments, the GSIS is static GSIS, optionally wherein the static incubation index is greater than at or about 1, greater than at or about 2, greater than at or about 5, greater than at or about 10 or greater than at or about 20.

In some of any embodiments, the level of insulin secretion by the engineered hypoimmunogenic islets is at least 20% of that observed for primary islets, optionally cadaveric islets. In some of any embodiments, the level of insulin secretion by the engineered hypoimmunogenic islets is at least 25%, at least 30%, at least 40%, at least 50%, at least 60%, at least 70% of that observed for primary islets, optionally cadaveric islets.

In some of any embodiments, the total insulin content of the engineered hypoimmunogenic islets is greater than at or about 500 μIU Insulin per 5000 cells, greater than at or about 1000 μIU Insulin per 5000 cells, greater than at or about 2000 μIU Insulin per 5000 cells, greater than at or about 3000 μIU Insulin per 5000 cells or greater than at or about 4000 μIU Insulin per 5000 cells. In some of any embodiments, the proinsulin to insulin ratio of the modified SC-beta cell is between at or about 0.02 and at or about 0.1, optionally at or about 0.03, 0.04, 0.05, 0.06, 0.07, 0.08, 0.09 and any value between any of the foregoing.

In some of any embodiments, the engineered hypoimmunogenic islets exhibits functionality for more than 2 weeks following transplantation into a subject. In some of any embodiments, the engineered hypoimmunogenic islets exhibits functionality for more than 3 weeks, for more than 4 weeks, for more than 8 weeks, for more than 3 months, for more than 6 months or for more than 12 months following transplantation into a subject.

In some of any embodiments, the functionality is selected from the group consisting of maintaining fasting blood glucose levels, secreting insulin in response to glucose injections in vivo, and clearing glucose after a glucose injection in vivo.

In some of any embodiments, the dose is from about 1×10⁷ cells to about 3×10⁸ cells. In some of any embodiments, the dose is from about 1.25×10⁵ cells/kg to about 1.2×10⁷ cells/kg.

In some of any embodiments, the dose is from about 6,500 islet equivalents (IEQ) to about 600,000 IEQ. In some of any embodiments, the dose is from about 80 IEQ/kg to about 24,000 IEQ/kg. In some of any embodiments, the subject is not administered an immunosuppression regimen.

### BRIEF DESCRIPTION OF THE DRAWINGS

FIGS. 1A-1B provide results of studies of allogeneic transplant studies evaluating non-human primate (NHP) recipient's immune response to the allogeneic NHP primary islet cells. Quantification of BLI of luciferase expression is provided for transplanted B2M^{-/-}; CIITA^{-/-}; CD47^{th} NHP primary islet cells (FIG. 1A quantification; FIG. 1B corresponding BLI image).

FIGS. 2A-2D provide results of i.m. injection allogeneic transplant studies in NHPs evaluating immune response. Interferon gamma (IFNg) levels are provided for NHPs transplanted with B2M^{-/-}; CIITA^{-/-}; CD47^{th} NHP primary islet cells (FIG. 2A). Donor-specific antibodies (DSA) IgM levels (FIG. 2B) and IgG levels (FIG. 2C) are provided NHPs transplanted with B2M^{-/-}; CIITA^{-/-}; CD47tg NHP primary islet cells. DSA IgG levels are also provided for a sensitized NHP transplanted with B2M^{-/-}; CIITA^{-/-}; CD47tg NHP primary islet cells with elevated IgG levels prior to transplantation (FIG. 2D).

FIG. 3 provides results of Natural Killer (NK) cell mediated cell killing in vitro of B2M^{-/-}CIITA^{-/-}; CD47tg NHP primary islet cells.

FIGS. 4A-4D show phenotyping and allogeneic transplantation of B2M^{-/-}; CIITA^{-/-}; CD47^{th} rhesus macaque primary islet cells. FIG. 4A shows immunofluorescence staining of somatostatin, insulin, and glucagon (top panel) and CD47, MHC class I, and DAPI (bottom panel) before and after B2M^{-/-}; CIITA^{-/-}; CD47^{th} editing. FIG. 4B shows MHC class I, MHC class II, and rhesus CD47 expression in rhesus macaque islets before and after B2M^{-/-}; CIITA^{-/-}; CD47^{th} editing. FIG. 4C shows insulin release from in vitro rhesus macaque islets before and after B2M^{-/-}; CIITA^{-/-}; CD47^{th} editing. FIG. 4D shows the composition of rhesus macaque islets before and after B2M^{-/-}; CIITA^{-/-}; CD47^{th} editing.

FIG. 5 shows blood glucose measurements for a diabetic non-human primate (NHP) transplanted with allogeneic B2M^{-/-}; CIITA^{-/-}; CD47tg NHP primary islet cells. Blood was collected in the morning (blood glucose AM) and in the afternoon (blood glucose PM). Diabetic: >127 mg/dL; Impaired fasting glucose: >80-127 mg/dL; Normal: <80 mg/dL; and Hypoglycemia: <30 mg/dL.

FIG. 6 shows blood glucose measurements for the diabetic non-human primate (NHP) transplanted with allogeneic B2M^{-/-}; CIITA^{-/-}; CD47tg NHP primary islet cells extended to day 111 post STZ. Blood was collected in the morning (blood glucose AM) and in the afternoon (blood glucose PM). Hyperglycemia (diabetic): >127 mg/dL; Impaired fasting glucose: >80-127 mg/dL; Normal: <80 mg/dL; and Hypoglycemia: <30 mg/dL.

FIG. 7 shows blood glucose measurements for the diabetic non-human primate (NHP) transplanted with allogeneic B2M^{-/-}; CIITA^{-/-}; CD47tg NHP primary islet cells extended to day 226 post STZ. Blood was collected in the morning (blood glucose AM) and in the afternoon (blood glucose PM). Hyperglycemia (diabetic): >127 mg/dL; Impaired fasting glucose: >80-127 mg/dL; Normal: <80 mg/dL; and Hypoglycemia: <30 mg/dL.

FIG. 8A shows administration of daily exogenous insulin (U/day) overtime. FIG. 8B shows morning and evening blood glucose levels (mg/dL) overtime. FIG. 8C shows serum c-peptide levels (ng/mL) over time. FIG. 8D shows weight (kg) overtime. Asterisks indicate c-peptide measurement time points.

FIG. 9 shows C-peptide measurements for a diabetic non-human primate (NHP) transplanted with allogeneic B2M^{-/-}; CIITA^{-/-}; CD47tg NHP primary islet cells. Pre-STZ: C-peptide measurement prior to i.v. injection of streptozotocin (STZ); d50 post STZ: C-peptide measurement on day 50 (d50) post STZ injection; d0 (d78 post STZ): C-peptide measurement on day 78 (d78) post STZ injection and day 0 of islet cell transplantation; d7 (d85 post STZ): C-peptide measurement on day 85 (d85) post STZ injection and day 7 post islet cell transplantation; d14 (d92 post STZ): C-peptide measurement on day 92 (d92) post STZ injection and day 14 (d14) post islet cell transplantation; d28 (d106 post STZ): C-peptide measurement on day 106 (d106) post STZ injection and day 28 (d28) post islet cell transplantation; d42 (d120 post STZ): C-peptide measurement on day 120 (d120) post STZ injection and day 42 (d42) post islet cell transplantation; d90 (d172 post STZ): C-peptide measurement on day 172 (d172) post STZ injection and day 90 (d90) post islet cell transplantation.

FIG. 10 shows glucose tolerance measurements for a diabetic non-human primate (NHP) transplanted with allogeneic B2M^{-/-}; CIITA^{-/-}; CD47tg NHP primary islet cells. Pre-STZ: glucose tolerance measurement prior to iv. injection of streptozotocin (STZ); d50 (post STZ): glucose tolerance measurement on day 50 (d50) post STZ injection; d103 (d25 after cell transplant): glucose tolerance measurement on day 103 (d103) post STZ injection and day 25 (d25) post islet cell transplantation; merged: Pre-STZ, d50, and d103.

FIGS. 11A-11L show cellular and antibody-mediated responses against B2M^{-/-}; CIITA^{-/-}; CD47^{th} rhesus macaque primary islet cells. FIG. 11A shows ELISpot assays with recipient monkey PBMCs drawn at scheduled timepoints. FIGS. 11B-11E show killing assays with recipient cynomolgus monkey T cells (FIG. 11B), PBMCs (FIG. 11C), NK cells (FIG. 11D) and macrophages (FIG. 11E). Percent target cell killing is shown on the y axis. FIGS. 11F-11I show Ig levels including total serum IgM (FIG. 11F), IgG (FIG. 11G), donor specific antibody (DSA) IgM (FIG. 11H) and DSA IgG (FIG. 11I). FIGS. 11J-11L show antibody-dependent cellular cytotoxicity (ADCC) assays with de-complemented recipient cynomolgus monkey serum and NK cells (FIG. 11J) or macrophages (FIG. 11K) and CDC assays with complete recipient monkey serum (FIG. 11L). Percent target cell killing is shown on the y axis.

FIGS. 12A and 12B show rhesus macaque B2M^{-/-}; CIITA^{-/-}; CD47^{th} primary islet cell killing by cynomolgus NK cells or macrophages in response to treatment with anti-CD47 antibody (magrolimab).

FIGS. 13A-13C show immunohistochemical stains of pancreas islets and the muscle primary islet transplantation site. FIG. 13A shows the pancreas from a healthy cynomolgus monkey. FIG. 13B shows the pancreas of the recipient cynomolgus monkey. FIG. 13C shows the muscular implant site of the recipient cynomolgus monkey.

### DETAILED DESCRIPTION

Provided herein are methods involving dosing a subject with engineered islets that include beta cells that are engineered to evade the immune system (also referred to herein as a modified immune-evasive beta cell or a hypoimmunogenic (HIP) beta cell). In some embodiments, the engineered islets can be engineered primary islets. In some embodiments, the engineered islets can be engineered islet cells that have been differentiated from pluripotent stem cells. In some embodiments, the engineered islet cells, including engineered beta cells, exhibit features that allow them to evade immune recognition. In some embodiments, the engineered islets cells, including engineered beta cells, are hypoimmunogenic (also referred to as hypoimmune or HIP. In some aspects, the engineered islet cells, including engineered beta cells, are not subject to an innate immune cell rejection. In some aspects, the engineered islets cells, including engineered beta cells, provided herein exhibit reduced innate immune cell rejection and/or adaptive immune cell rejection (e.g. hypoimmunogenic cells). For example, in some embodiments, the engineered islet cells, including engineered beta cells, exhibit reduced susceptibility to NK cell-mediated lysis and/or macrophage engulfment. In some embodiments, the engineered islets and cells are useful as a source of universally compatible cells or tissues (e.g. universal donor cells or tissues) that are transplanted into a recipient subject. Such hypoimmunogenic cells retain cell-specific characteristics and features upon administration to a subject (e.g. transplantation or engraftment). In some embodiments, the engineered islet cells cluster into effective endocrine organoids, termed pseudo islet grafts (p-islets), when transplanted or engrafted in a subject. Thus, in some embodiments, the engineered islets are HIP pseudo-islets (HIP p-islets). In some embodiments, an effective endocrine organoid provides stable endocrine function via production and secretion of insulin, thereby enabling insulin independence in the subject. In some embodiments, stable endocrine function and insulin independence occurs in the absence of immunosuppression. In some embodiments, the engineered islet cells, including engineered beta cells, can be used as a source of cells for allogeneic therapy regardless of the subject's genetic make-up.

In some embodiments, the provided methods are for treating a beta cell related disorder (e.g. diabetes) in a subject, such as to improve glucose tolerance in the subject. In particular embodiments, the methods are for treating Type I diabetes in a subject, such as to improve glucose tolerance in the subject. In other embodiments, the methods improve graft function of the provided islet cells. In some embodiments, the methods restore glucose metabolism in a subject.

Patients with type 1 diabetes mellitus (T1DM) or impaired awareness of hypoglycemia (IAH) lack basic hypoglycemia-induced defense mechanisms, and are thus at increased risk for severe hypoglycemic events (Hwang et al., J Clin Invest (2018) 128:1485-195; Lin et al., J Diabetes Investig (2020) 11:1388-1402). Current therapies for T1DM patients include intensive insulin treatment. However, these treatments can lead to sever hypoglycemia, which is associated with altered mental state, seizures, cardiac arrhythmias and even death (Bornstein et al., Nat Rev Endocrinol (2022) 18:389-390).

Pancreatic islet transplantation has been shown to be superior to insulin therapies, with improved patient survival and quality of life (Boughton et al., Diabetes Obes Metab (2021) 23:1389-1396). However, transplantation of pancreatic islets in patients with T1DM is severely hampered by the requirement for continuous immunosuppression. Systemic immunosuppression to prevent the rejection of allogeneic islet grafts in patients comes with considerable morbidity, including chronic kidney injury, infections and cancer, and a graft survival of only 4.4 to 5.9 years (Hering et al., Diabetes Care (2016) 39:1230-1240; Lemos et al., Diabetes Care (2021) 44:e67-e68; Marfil-Garza et al., Lancet Diabetes Endocrinol (2022) 10:519-532). Moreover, despite receiving immunosuppression, T1DM patients frequently become sensitized to the allogeneic transplant and develop elevated panel reactive antibodies, complicating any subsequent transplants. There is thus a need for improved methods for pancreatic islet transplantation, including for treating diabetes.

The provided embodiments address these needs. The provided embodiments relate to primary islets that have been engineered to be hypoimmune, thereby reducing or eliminating the need for immunosuppression. Particularly, results herein establish that allogeneic transplantation of primary, hypoimmune engineered, beta islet cells into a fully immunocompetent, diabetic non-human primate model provided stable endocrine function, and enabled insulin independence without inducing any detectable immune response in the absence of immunosuppression. Thus, the present disclosure demonstrates that hypoimmune primary beta islet cells provide a novel and curative cell therapy for T1DM, and can do so with reduced or no requirement for immunosuppression.

In some embodiments, the engineered islets, including engineered beta cells, described herein are hypoimmunogenic when administered (e.g. transplanted or grafted), and in some embodiments, evade immune rejection. Non-limiting examples of modifications that result in evading immune rejection include reduced expression of major histocompatibility complex (MHC) human leukocyte antigen (HLA) class I antigens and HLA class II antigens, and increased expression of one or more tolerogenic factors, such as CD47. In some embodiments, the engineered islets, including engineered beta cells, are administered in an MHC-mismatched allogenic subject.

In some embodiments, the engineered islet cells, including engineered beta cells, contain modifications that (a) reduce expression of one or more major histocompatibility complex (MHC) class I molecules and/or one or more of MHC class II molecules; and (b) increase expression of one or more tolerogenic factors in the engineered islets, relative to a control or a wild-type beta cell. In some embodiments, the modifications make the cells hypoimmune, which in some aspects allow the cells to evade immune rejections compared to control or wild-type islet cells, such as primary human islet cells beta cells. For purposes herein, the terms engineered islets can be used interchangeably with the term hypoimmune derived islets.

The engineered islets include engineered cells, such as engineered beta cells, that utilize expression of tolerogenic factors and are also modulated (e.g. reduced or eliminated) for expression (e.g. surface expression) of one or more MHC class I molecules and/or one or more MHC class II molecules. In some embodiments, the modification that reduces expression of one or more MHC class I molecules is a modification that reduces expression of β-2 microglobulin (B2M). In some embodiments, the modification that reduces expression of one or more MHC class II molecules is a modification that reduces expression of CIITA. In some embodiments, the engineered cells comprising the modifications described herein (including reduced or eliminated expression of MHC class I molecules or MHC class II molecules and increased expression of CD47 or other tolerogenic factor) survive, engraft, persist, and function following administration (e.g. transplant or engraftment). In some embodiments, cells of the engineered islets exhibit enhanced survival and/or enhanced engraftment and/or function for a longer term in comparison to control or wild-type islets, such as unmodified islet cells that do not comprise the modifications rendering the cells hypoimmune.

In some embodiments, the engineered islets are administered via intramuscular injection (e.g. intramuscular injection to the forearm).

In some embodiments, genome editing technologies utilizing rare-cutting endonucleases (e.g. the CRISPR/Cas, TALEN, zinc finger nuclease, meganuclease, and homing endonuclease systems) are used to reduce or eliminate expression of immune genes (e.g. by deleting genomic DNA of critical immune genes) as described herein, such as genes involved in regulating expression of MHC class I molecules or MHC class II molecules, in islet cells used to derive the engineered islets. In certain embodiments, genome editing technologies or other gene modulation technologies are used to insert tolerance-inducing (tolerogenic) factors (e.g. CD47) into a target genomic locus of islet cells used to derive the engineered islets, thus producing engineered islets that can evade immune recognition upon engrafting into a recipient subject. Therefore, the engineered islets exhibit modulated expression (e.g. reduced or eliminated expression) of one or more genes and factors that affect expression of MHC class I molecules and/or MHC class II molecules, modulated expression (e.g. reduced or and modulated expression (e.g. overexpression) of tolerogenic factors, such as CD47, and provide for reduced recognition by the recipient subject's immune system. In some embodiments, the modified cells can also exhibit modulated expression (e.g. reduced expression) of CD142, which, in some aspects, can also be reduced by genome editing technologies (e.g. the CRISPR/Cas, TALEN, zinc finger nuclease, meganuclease, and homing endonuclease systems) to reduce or eliminate expression of CD142 (e.g. by deleting genomic DNA of critical immune genes). In some embodiments, the engineered islets can exhibit modulated expression (e.g. increased expression) of one or more complement inhibitors selected from CD46, CD59, CD55 and CD35, which, in some aspects, can also be increased by genome editing technologies to insert or integrate an exogenous polynucleotide encoding the one or more complement inhibitors into a genomic locus in the engineered islets.

In some embodiments, the beta cell related disorder is a metabolic disorder. In some embodiments, the metabolic disorder is familial hypercholesterolemia, Gaucher disease, Hunter syndrome, Krabbe disease, maple syrup urine disease, metachromatic leukodystrophy, mitochondrial encephalopathy, lactic acidosis, stroke-like episodes (MELAS), Niemann-Pick disease, phenylketonuria (PKU), porphyria, Tay-Sachs disease, Wilson's disease, Type I diabetes, Type II diabetes, obesity, hypertension, dyslipidemia, or carbohydrate intolerance. In some embodiments, the beta cell related disorder is Type I diabetes.

The practice of the particular embodiments will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. See e.g. Sambrook, et al., Molecular Cloning: A Laboratory Manual (3rd Edition, 2001); Sambrook, et al., Molecular Cloning: A Laboratory Manual (2nd Edition, 1989); Maniatis et al., Molecular Cloning: A Laboratory Manual (1982); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford, 1985); Anand, Techniques for the Analysis of Complex Genomes, (Academic Press, New York, 1992); Transcription and Translation (B. Hames & S. Higgins, Eds., 1984); Perbal, A Practical Guide to Molecular Cloning (1984); Harlow and Lane, Antibodies, (Cold Spring Harbor Laboratory Press, Cold Spring Harbor, N.Y., 1998) Current Protocols in Immunology Q. E. Coligan, A. M. Kruisbeek, D. H. Margulies, E. M. Shevach and W. Strober, eds., 1991); Annual Review of Immunology; as well as monographs in journals such as Advances in Immunology.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described. Those skilled in the art will recognize that several embodiments are possible within the scope and spirit of the present disclosure. The following description illustrates the disclosure and, of course, should not be construed in any way as limiting the scope of the inventions described herein.

### I. Methods and Dosing of a Beta Cell Therapy

In some aspects, provided herein is a method of treating a beta cell related disorder in a subject, the method comprising administering to a subject engineered islets as described. The engineered islets administered to a subject according to the methods provided herein include cells that have been modified to evade immune rejection. In some embodiments, the engineered islets are administered as an islet cluster. In particular embodiments, the engineered islets include engineered beta cells. In some embodiments, the engineered beta cell is in a composition comprising additional islet cells. In some embodiments, the islets, such as islet cluster, further comprises alpha cells and/or delta cells. In some embodiments, the islets, such as islet cluster, further comprises epsilon cells and/or PP cells. In some embodiments, cells of the engineered islets include the same hypoimmune modifications. In particular embodiments, cells of the engineered islets include beta cells modified with hypoimmune modifications. Exemplary features of the engineered islets, including engineered or engineered islets, for use in the provided methods are described in Section II.

The engineered cells provided herein can be administered to a subject for the treatment of a beta cell related disease or disorder. In some embodiments, the subject is a mammal. In some embodiments, the subject is a human.

In some embodiments, the beta cell related disorder is a metabolic disorder. A metabolic disorder may occur when abnormal chemical reactions in the body of a subject disrupts metabolic processes (e.g. processes related to the metabolism, or breakdown, of energy into sugars and acids or the storage of said energy). In some embodiments, the metabolic disorder affects the breakdown of amino acids, carbohydrates, or lipids in a subject's body. In some embodiments, the metabolic disorder affects the subject's mitochondria (e.g. mitochondrial diseases). In some embodiments, the metabolic disorder develops when the subject's organs, such as the liver or pancreas, become disease and/or do not function normally. Exemplary metabolic disorders herein may comprise, but are not limited to, any disease or disorder characterized by increased blood pressure, high blood sugar, excess body fat around the waist, and abnormal cholesterol or triglyceride levels. In some embodiments, the metabolic disorder is familial hypercholesterolemia, Gaucher disease, Hunter syndrome, Krabbe disease, maple syrup urine disease, metachromatic leukodystrophy, mitochondrial encephalopathy, lactic acidosis, stroke-like episodes (MELAS), Niemann-Pick disease, phenylketonuria (PKU), porphyria, Tay-Sachs disease, Wilson's disease, Type I diabetes, Type II diabetes, obesity, hypertension, dyslipidemia, or carbohydrate intolerance. In some embodiments, the metabolic disorder is Type II diabetes. In some embodiments, the metabolic disorder is Type I diabetes. In some embodiments, the metabolic disorder is Type I diabetes mellitus.

In some embodiments, the beta cell disorder is a metabolic disorder. In some embodiments, the metabolic disorder is selected from the group consisting of: familial hypercholesterolemia, Gaucher disease, Hunter syndrome, Krabbe disease, maple syrup urine disease, metachromatic leukodystrophy, mitochondrial encephalopathy, lactic acidosis, stroke-like episodes (MELAS), Niemann-Pick disease, phenylketonuria (PKU), porphyria, Tay-Sachs disease, Wilson's disease, Type I diabetes, Type II diabetes, obesity, hypertension, dyslipidemia, and carbohydrate intolerance. In some embodiments, the disorder is diabetes. In some embodiments, the disorder is Type I diabetes.

### A. Islet Cells

In some embodiments, the engineered islets, including engineered beta cells, have the ability to evade the immune system. In some embodiments, the engineered islets, including engineered beta cells, comprises modifications that: (a) reduce expression of one or more of major histocompatibility complex (MHC) class I molecules and/or one or more of MHC class II molecules in the engineered islets, relative to a control or wild-type islet cell; and (b) increase expression of one or more tolerogenic factors in the engineered cell, relative to the control or wild-type islet cell, such as relative to the control or wild-type beta cell. In some embodiments, the engineered islets, including engineered beta cells, comprise modifications that reduce expression of B2M in the engineered cell, relative to the control or wild-type islet cell, such as control or wild-type beta cell. In some embodiments, the engineered islet cell comprises modifications that reduce expression of CIITA in the modified islet cell, relative to the control or wild-type islet cell, such as relative to the control or wild-type beta cell. In some embodiments, the engineered islet cell comprises modifications that increase expression of CD47 in the engineered islet cell, relative to the control or wild-type islet cell, such as relative to the control or wild-type beta cell. In some embodiments, the engineered islet cells, such as engineered beta cell, comprises modifications that: (a) reduce expression of B2M, relative to a control or wild-type islet cell; (b) reduce expression of CIITA, relative to a control or wild-type islet cell; and (c) increase expression of CD47 in the engineered islet cell, relative to the control or wild-type islet cell.

In some embodiments, the islets are primary islets that have been engineered with a hypoimmune modification as described. In some embodiments, the primary islets are human. In some embodiments, the islet cells, including beta cells, are cells that have been differentiated from stem cells and that are engineered with a hypoimmune modification as described. In some embodiments, the stem cell is selected from the group consisting of a pluripotent stem cell (PSC), an induced pluripotent stem cell (iPSC), an embryonic stem cell, a hematopoietic stem cell, a mesenchymal stem cell, an endothelial stem cell, an epithelial stem cell, an adipose stem cell, a germline stem cell, a lung stem cell, a cord blood stem cell, and a multipotent stem cell. In some embodiments, the stem cell is a pluripotent stem cell (PSC). In some embodiments, the stem cell is an induced pluripotent stem cell (iPSC), mesenchymal stem cell (MSC), hematopoietic stem cell (HSC), or embryonic stem cell (ESC). In some embodiments, the stem cell is in a suspension.

In some embodiments, the islets cells are primary islet cells (also referred to as pancreatic islet cells). In particular embodiments, the primary islet cells include primary beta islet cells (pancreatic beta islet cells). In some embodiments, the primary islets are isolated or obtained from one or more individual donor subjects, such as one or more individual healthy donor (e.g. a subject that is not known or suspected of, e.g. not exhibiting clinical signs of, a disease or infection). In some embodiments, the donor is a cadaver. As will be appreciated by those in the art, methods of isolating or obtaining islets from an individual can be achieved using known techniques.

In some embodiments, islet cells are obtained (e.g., harvested, extracted, removed, or taken) from a subject or an individual. In some embodiments, primary islet cells are produced from a pool of islet cells such that the islet cells are from one or more subjects (e.g., one or more human including one or more healthy humans). In some embodiments, the pool of primary islet cells is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects. In some embodiments, the donor subject is different from the patient (e.g. the recipient subject that is administered the therapeutic cells). In some embodiments, the pool of islet cells does not include cells from the patient. In some embodiments, one or more of the donor subjects from which the pool of islets cells is obtained are different from the patient.

Additional descriptions of pancreatic islet cells including for use in the present technology are found in WO2020/018615, the disclosure is herein incorporated by reference in its entirety.

In some embodiments, the population of engineered primary islet cells, including primary beta islet cells, isolated from one or more individual donors (e.g. healthy donors) are maintained in culture, in some cases expanded, prior to administration. In certain embodiments, the population of engineered islet cells are cryopreserved prior to administration.

Exemplary pancreatic islet cell types include, but are not limited to, pancreatic islet progenitor cell, immature pancreatic islet cell, mature pancreatic islet cell, and the like. In some embodiments, pancreatic cells described herein are administered to a subject to treat diabetes.

In some embodiments, the pancreatic islet cells disclosed herein, such as primary beta islet cells isolated from one or more individual donors (e.g. healthy donors), secretes insulin. In some embodiments, a pancreatic islet cell exhibits at least two characteristics of an endogenous pancreatic islet cell, for example, but not limited to, secretion of insulin in response to glucose, and expression of beta islet cell markers.

Exemplary beta islet cell markers or beta islet cell progenitor markers include, but are not limited to, c-peptide, Pdxl, glucose transporter 2 (Glut2), HNF6, VEGF, glucokinase (GCK), prohormone convertase (PC 1/3), Cdcpl, NeuroD, Ngn3, Nkx2.2, Nkx6.1, Nkx6.2, Pax4, Pax6, Ptfla, Isll, Sox9, Sox17, and FoxA2.

In some embodiments, the primary pancreatic islet cells may be isolated from a primary pancreatic islet, derived from primary pancreatic islet cells within a primary pancreatic islet, or as a component of a primary pancreatic islet. For example, primary pancreatic beta islet cells can be edited as a single beta islet cell, a population of beta islet cells, or as a component of a primary pancreatic islet (e.g., primary pancreatic beta islet cells present within the primary pancreatic islet along with other cell types). As another example, primary pancreatic beta islet cells can be administered to a patient as single beta islet cells, a population of beta islet cells, or as a component of a primary pancreatic islet (e.g., primary pancreatic beta islet cells present within the primary pancreatic islet along with other cell types). In embodiments where the pancreatic beta islet cells are present within the pancreatic islet along with other cell types, the other cell types may also be edited by the methods described herein.

In some embodiments, the primary pancreatic islet cells are dissociated from a primary islet prior to or after engineering, such as genetic engineering. Such dissociated islet cells can be clustered prior to administration to a patient and clusters can include beta islet cells as well as other cell types including but not limited to those from the primary islet. Numbers of islet cells in the cluster can vary, such as about 50, about 100, about 250, about 500, about 750, about 1000, about 1250, about 1500, about 1750, about 2000, about 2250, about 2500, about 2750, about 3000, about 3500, about 4000, about 4500, or about 5000 cells. Patients can be administered about 10, about 20, about 30, about 40, about 50, about 75, about 100, about 125, about 150, about 200, about 250, about 300, about 325, about 350, about 375, about 400, about 425, about 450, about 475, about 500, about 600, about 700, about 800, about 900, or about 1000 clusters.

In some embodiments, the primary pancreatic islet cells, isolated from one or more individual donors (e.g., healthy donors), produce insulin in response to an increase in glucose. In some embodiments, the pancreatic islet cells are beta islet cells. In some embodiments, the beta islet cells are monitored to assess glucose control abilities. Assays to monitor glucose control may include, but are not limited to, continuous blood glucose level monitoring, monitoring blood glucose levels after a period of fasting, glucose tolerance (e.g., glucose challenge) tests, glucose utilization and oxidation, insulin secretion, such as by a U-PLEX® Meso Scale Discovery (MSD) assay and/or glucose-stimulated insulin secretion (GSIS) assays, measuring the presence of specific transcription factors and pathways (e.g., homeobox transcription factor SIX2, NKX6-1, and PDX1), measuring mitochondrial respiration, and measuring changes in intracellular Ca2+ calcium flux, such as glucose-induced Ca2+ rise, Ca2+-activated exocytosis. Various methods of measuring glucose control are known in the art, such as those described in Velazco-Cruz et al., Cell Reports, 2020, 31, 107687; Pagliuca et al., Cell, 2014, 159(2): 428-439; Davis et al., Cell Reports, 2020, 31(6): 107623; and Alcazar et al., Cell Transplantation, 2020, 29, the disclosures including the figures, figure legends, and description of methods are incorporated herein by reference in their entirety. In some embodiments, the beta islet cells (e.g., modified beta islet cells) may exhibit GSIS. In some embodiments, the GSIs measured in a perfusion GSIS assay. In some embodiments, the GSIs dynamic GSIS comprising first and second phase dynamic insulin secretion. In some embodiments, the GSIs static GSIS. For example, the static incubation index may be greater than at or about 1, greater than at or about 2, greater than at or about 5, greater than at or about 10 or greater than at or about 20. In various embodiments, the pancreatic islet cells secrete insulin in response to an increase in glucose. In some embodiments, the cells have a distinct morphology such as a cobblestone cell morphology and/or a diameter of about 17 μm to about 25 pm.

In some embodiments, the cell used to generate the engineered islet cell is a stem or progenitor cell that is capable of being differentiated (e.g. the stem cell is totipotent, pluripotent, or multipotent). In some embodiments, the cell isolated from embryonic or neonatal tissue. In some embodiments, the cell is an embryonic stem cell. In some embodiments, the cell is an induced pluripotent stem cell derived from somatic cells (e.g. skin or blood cells) and reprogrammed into an embryonic-like pluripotent state. In some embodiments, the induced pluripotent stem cell is derived from a fibroblast. In some embodiments, the cells that are modified as provided herein are pluripotent stems cells or are cells differentiated from pluripotent stem cells. The cell may be a vertebrate cell, for example, a mammalian cell, such as a human cell or a mouse cell. The cell may also be a vertebrate stem cell, for example, a mammalian stem cell, such as a human stem cell or a mouse stem cell. In embodiments, the cell or stem cell is amenable to modification. The cell or stem cell, or a cell derived from such a stem cell, can have therapeutic value, such that the cell or stem cell or a cell derived or differentiated from such stem cell may be used to treat a disease, disorder, defect or injury in a subject in need of treatment for same.

In some embodiments, the islet cells, including beta cells, that are modified or engineered as provided herein are modified pluripotent stem cells (e.g. modified iPSC). The generation of mammalian (e.g. mouse and human) pluripotent stem cells (generally referred to as iPSCs; miPSCs for murine cells or hiPSCs for human cells) is generally known in the art. As will be appreciated by those in the art, there are a variety of different methods for the generation of iPSCs. The original induction was done from mouse embryonic or adult fibroblasts using the viral introduction of four transcription factors, Oct3/4, Sox2, c-Myc and Klf4; see Takahashi and Yamanaka Cell 126:663-676 (2006), hereby incorporated by reference in its entirety and specifically for the techniques outlined therein. Since then, a number of methods have been developed; see Seki et al, World J. Stem Cells 7(1): 116-125 (2015) for a review, and Lakshmipathy and Vermuri, editors, Methods in Molecular Biology: Pluripotent Stem Cells, Methods and Protocols, Springer 2013, both of which are hereby expressly incorporated by reference in their entirety, and in particular for the methods for generating hiPSCs (see for example Chapter 3 of the latter reference).

Generally, iPSCs are generated by the transient expression of one or more reprogramming factors” in the host cell, usually introduced using episomal vectors. Under these conditions, small amounts of the cells are induced to become iPSCs (in general, the efficiency of this step is low, as no selection markers are used). Without wishing to be bound by theory, it is believed that once the cells are “reprogrammed”, and become pluripotent, they lose the episomal vector(s) and produce the factors using the endogenous genes.

As is also appreciated by those of skill in the art, the number of reprogramming factors that can be used or are used can vary. Commonly, when fewer reprogramming factors are used, the efficiency of the transformation of the cells to a pluripotent state goes down, as well as the “pluripotency”, e.g. fewer reprogramming factors may result in cells that are not fully pluripotent but may only be able to differentiate into fewer cell types.

In some embodiments, a single reprogramming factor, OCT4, is used. In other embodiments, two reprogramming factors, OCT4 and KLF4, are used. In other embodiments, three reprogramming factors, OCT4, KLF4 and SOX2, are used. In other embodiments, four reprogramming factors, OCT4, KLF4, SOX2 and c-Myc, are used. In other embodiments, 5, 6 or 7 reprogramming factors can be used selected from SOKMNLT; SOX2, OCT4 (POU5F1), KLF4, MYC, NANOG, LIN28, and SV40L T antigen. In general, these reprogramming factor genes are provided on episomal vectors such as are known in the art and commercially available.

In some embodiments, the host cells used for transfecting the one or more reprogramming factors are non-pluripotent stem cells. In general, as is known in the art, iPSCs are made from non-pluripotent cells such as, but not limited to, blood cells, fibroblasts, etc., by transiently expressing the reprogramming factors as described herein. In some embodiments, the non-pluripotent cells, such as fibroblasts, are obtained or isolated from one or more individual subjects or donors prior to reprogramming the cells. In some embodiments, iPSCs are made from a pool of isolated non-pluripotent stems cells, e.g. fibroblasts, obtained from one or more (e.g. two or more, three or more, four or more, five or more, ten or more, twenty or more, fifty or more, or one hundred or more) different donor subjects. In some embodiments, the non-pluripotent cells, such as fibroblasts, are isolated or obtained from a plurality of different donor subjects (e.g. two or more, three or more, four or more, five or more, ten or more, twenty or more, fifty or more, or one hundred or more), pooled together in a batch, reprogrammed as iPSCs and are modified in accord with the provided methods.

In some embodiments, the iPSCs are derived from, such as by transiently transfecting one or more reprogramming factors into cells from a pool of non-pluripotent cells (e.g. fibroblasts) from one or more donor subjects that are different than the recipient subject (e.g. the patient administered the cells). The non-pluripotent cells (e.g. fibroblasts) to be induced to iPSCs can be obtained from 1, 2, 3, 4, 5, 6, 7, 8, 9, 10, 20, 50, 100 or more donor subjects and pooled together. The non-pluripotent cells (e.g. fibroblasts) can be obtained from 1 or more, 2 or more, 3 or more, 4 or more, 5 or more, 6 or more, 7 or more, 8 or more, 9 or more, 10, or more 20 or more, 50 or more, or 100 or more donor subjects and pooled together. In some embodiments, the non-pluripotent cells (e.g. fibroblasts) are harvested from one or a plurality of individuals, and in some instances, the non-pluripotent cells (e.g. fibroblasts) or the pool of non-pluripotent cells (e.g. fibroblasts) are cultured in vitro and transfected with one or more reprogramming factors to induce generation of iPSCs. In some embodiments, the non-pluripotent cells (e.g. fibroblasts) or the pool of non-pluripotent cells (e.g. fibroblasts) are modified in accord with the methods provided herein. In some embodiments, the modified iPSCs or a pool of modified iPSCs are then subjected to a differentiation process for differentiation into any cells of an organism and tissue.

The PSCs can be differentiated into beta cells of an organism and tissue. In an aspect, provided herein are modified cells that are differentiated into beta cells from iPSCs for after administration into recipient subjects. Differentiation can be assayed as is known in the art, generally by evaluating the presence of cell-specific markers. As will be appreciated by those in the art, the differentiated modified (e.g. hypoimmunogenic) pluripotent cell derivatives can be transplanted using techniques known in the art that depends on both the cell type and the ultimate use of these cells. Exemplary types of differentiated cells and methods for producing the same are described below. In some embodiments, the iPSCs may be differentiated to beta cells. In some embodiments, the iPSCs are differentiated into beta islet cells. In some embodiments, host cells such as non-pluripotent cells (e.g. fibroblasts) from an individual donor or a pool of individual donors are isolated or obtained, generated into iPSCs in which the iPSCs are then modified to contain modifications (e.g. genetic modifications) described herein and then differentiated into a desired cell type.

In some embodiments, the cells are beta islet cells derived from modified iPSCs that contain modifications (e.g. genetic modifications) described herein and that are differentiated into beta islet cells. As will be appreciated by those in the art, the methods for differentiation depend on the desired cell type using known techniques. In some embodiments, the cells differentiated into various beta islet cells may be used for after transplantation or engraftment into subjects (e.g. recipients). In some embodiments, pancreatic islet cells are derived from the modified pluripotent cells described herein. Useful methods for differentiating pluripotent stem cells into beta islet cells are described, for example, in U.S. Pat. Nos. 9,683,215; 9,157,062; 8,927,280; U.S. Patent Pub. No. 2021/0207099; Hogrebe et al., “Targeting the cytoskeleton to direct pancreatic differentiation of human pluripotent stem cells,” Nat. Biotechnol., 2020, 38:460-470; and Hogrebe et al., “Generation of insulin-producing pancreatic beta cells from multiple human stem cell lines,” Nat. Protoc., 2021, the contents of which are herein incorporated by reference in their entirety,

In some embodiments, the modified pluripotent cells described herein are differentiated into beta-like cells or islet organoids for transplantation to address type I diabetes mellitus (T1DM). Cell systems are a promising way to address T1DM, see, e.g. Ellis et al, Nat Rev Gastroenterol Hepatol. 2017 Oct; 14(10):612-628, incorporated herein by reference. Additionally, Pagliuca et al. (Cell, 2014, 159(2):428-39) reports on the successful differentiation of beta-cells from hiPSCs, the contents incorporated herein by reference in its entirety and in particular for the methods and reagents outlined there for the large-scale production of functional human beta cells from human pluripotent stem cells). Furthermore, Vegas et al. shows the production of human beta cells from human pluripotent stem cells followed by encapsulation to avoid immune rejection by the host; Vegas et al., Nat Med, 2016, 22(3):306-11, incorporated herein by reference in its entirety and in particular for the methods and reagents outlined there for the large-scale production of functional human 3 cells from human pluripotent stem cells.

In some embodiments, the method of producing a population of modified pancreatic islet cells from a population of modified pluripotent cells by in vitro differentiation comprises: (a) culturing the population of modified iPSCs in a first culture medium comprising one or more factors selected from the group consisting insulin-like growth factor, transforming growth factor, FGF, EGF, HGF, SHH, VEGF, transforming growth factor-b superfamily, BMP2, BMP7, a GSK inhibitor, an ALK inhibitor, a BMP type 1 receptor inhibitor, and retinoic acid to produce a population of immature pancreatic islet cells; and (b) culturing the population of immature pancreatic islet cells in a second culture medium that is different than the first culture medium to produce a population of modified pancreatic islet cells. In some embodiments, the GSK inhibitor is CHIR-99021, a derivative thereof, or a variant thereof. In some instances, the GSK inhibitor is at a concentration ranging from about 2 mM to about 10 mM. In some embodiments, the ALK inhibitor is SB-431542, a derivative thereof, or a variant thereof. In some instances, the ALK inhibitor is at a concentration ranging from about 1 pM to about 10 pM. In some embodiments, the first culture medium and/or second culture medium are absent of animal serum.

Differentiation is assayed as is known in the art, generally by evaluating the presence of β cell associated or specific markers, including but not limited to, insulin. Differentiation can also be measured functionally, such as measuring glucose metabolism, see generally Muraro et al., Cell Syst. 2016 Oct. 26; 3(4): 385-394.e3, hereby incorporated by reference in its entirety, and specifically for the biomarkers outlined there. Once the beta cells are generated, they can be transplanted (either as a cell suspension, cell clusters, or within a permeable or semipermeable device or gel matrix as discussed herein) into the portal vein/liver, the omentum, the gastrointestinal mucosa, the bone marrow, a muscle, or subcutaneous pouches.

In some embodiments, the pancreatic islet cells, such as beta islet cells differentiated from iPSCs derived from one or more individual donors (e.g. healthy donors), produce insulin in response to an increase in glucose. In various embodiments, the pancreatic islet cells secrete insulin in response to an increase in glucose. In some embodiments, the cells have a distinct morphology such as a cobblestone cell morphology and/or a diameter of about 17 pm to about 25 pm.

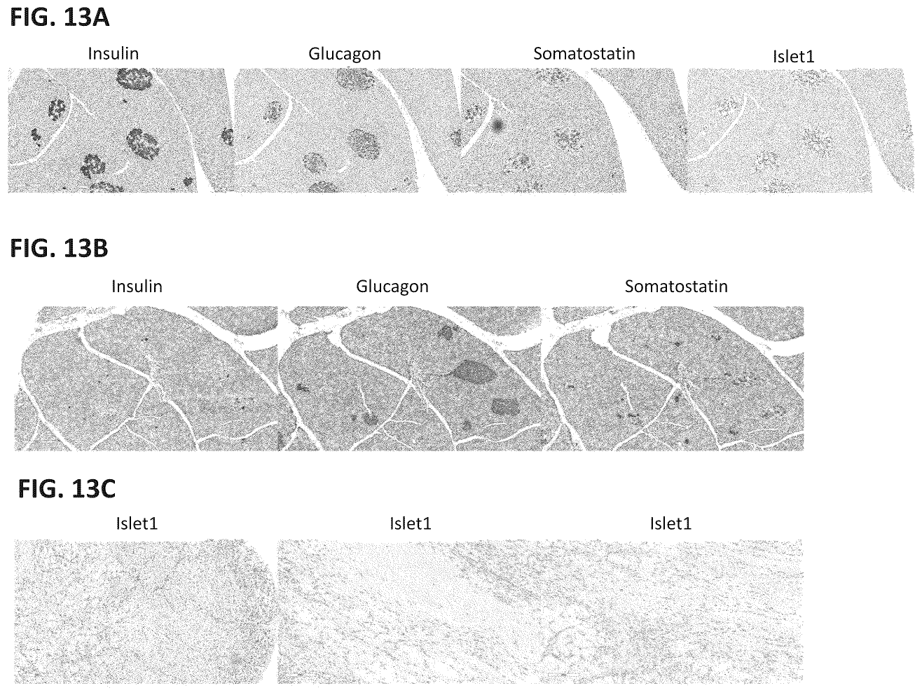

Once the engineered islets have been generated, they may be assayed for their hypoimmunogenicity and/or retention of pluripotency as is described in WO2016183041 and WO2018132783. In some embodiments, hypoimmunogenicity is assayed using a number of techniques as exemplified in FIG. 13 and FIG. 15 of WO2018132783. These techniques include transplantation into allogeneic hosts and monitoring for hypoimmunogenic pluripotent cell growth (e.g. teratomas) that escape the host immune system. In some instances, hypoimmunogenic pluripotent cell derivatives are transduced to express luciferase and can then followed using bioluminescence imaging. Similarly, the T cell and/or B cell response of the host animal to such cells are tested to confirm that the cells do not cause an immune reaction in the host animal. T cell responses can be assessed by Elispot, ELISA, FACS, PCR, or mass cytometry (CYTOF). B cell responses or antibody responses are assessed using FACS or Luminex. Additionally or alternatively, the cells may be assayed for their ability to avoid innate immune responses, e.g. NK cell killing, as is generally shown in FIGS. 14 and 15 of WO2018132783.

In some embodiments, the immunogenicity of the cells is evaluated using T cell immunoassays such as T cell proliferation assays, T cell activation assays, and T cell killing assays recognized by those skilled in the art. In some cases, the T cell proliferation assay includes pretreating the cells with interferon-gamma and coculturing the cells with labelled T cells and assaying the presence of the T cell population (or the proliferating T cell population) after a preselected amount of time. In some cases, the T cell activation assay includes coculturing T cells with the cells outlined herein and determining the expression levels of T cell activation markers in the T cells.

In vivo assays can be performed to assess the immunogenicity of the cells outlined herein. In some embodiments, the survival and immunogenicity of modified iPSCs is determined using an allogeneic humanized immunodeficient mouse model. In some instances, the modified iPSCs are transplanted into an allogeneic humanized NSG-SGM3 mouse and assayed for cell rejection, cell survival, and teratoma formation. In some instances, grafted modified iPSCs or differentiated cells thereof display long-term survival in the mouse model.

Additional techniques for determining immunogenicity including hypoimmunogenicity of the cells are described in, for example, Deuse et al., Nature Biotechnology, 2019, 37, 252-258 and Han et al., Proc Natl Acad Sci USA, 2019, 116(21), 10441-10446, the disclosures including the figures, figure legends, and description of methods are incorporated herein by reference in their entirety.

Similarly, the retention of pluripotency may be tested in a number of ways. In one embodiment, pluripotency is assayed by the expression of certain pluripotency-specific factors as generally described herein and shown in FIG. 29 of WO2018132783. Additionally or alternatively, the pluripotent cells are differentiated into one or more cell types as an indication of pluripotency.

Once the modified pluripotent stem cells (modified iPSCs) have been generated, they can be maintained in an undifferentiated state as is known for maintaining iPSCs. For example, the cells can be cultured on Matrigel using culture media that prevents differentiation and maintains pluripotency. In addition, they can be in culture medium under conditions to maintain pluripotency.

### B. Compositions and Formulations

In some aspects, the engineered beta islets are provided as a pharmaceutical composition for administration to the subject. In some embodiments, the pharmaceutical composition comprises a engineered islets and a pharmaceutically acceptable carrier.

Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polysorbates (TWEEN™), poloxamers (PLURONICS™) or polyethylene glycol (PEG). In some embodiments, the pharmaceutical composition includes a pharmaceutically acceptable buffer (e.g. neutral buffer saline or phosphate buffered saline). In some embodiments, the pharmaceutical composition can contain one or more excipients for modifying, maintaining or preserving, for example, the pH, osmolarity, viscosity, clarity, color, isotonicity, odor, sterility, stability, rate of dissolution or release, adsorption, or penetration of the composition. In some aspects, a skilled artisan understands that a pharmaceutical composition containing cells may differ from a pharmaceutical composition containing a protein.

The pharmaceutical composition in some embodiments contains engineered islets as described herein in amounts effective to treat or prevent the beta cell associated disease or disorder, such as a therapeutically effective or prophylactically effective amount. In some embodiments, the pharmaceutical composition contains engineered islets as described herein in amounts effective to treat or prevent the beta cell associated disease or disorder, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In some embodiments, the engineered islets are administered using standard administration techniques, formulations, and/or devices. In some embodiments, the engineered islets or composition or a population thereof as described herein are administered using standard administration techniques, formulations, and/or devices. Provided are formulations and devices, such as syringes and vials, for storage and administration of the compositions. The engineered islets can be administered via localized injection, including catheter administration, systemic injection, localized injection, intravenous injection, or parenteral administration. When administering a therapeutic composition, such as containing engineered islets, it will generally be formulated in a unit dosage injectable form (solution, suspension, emulsion).

Formulations include those for intravenous, intraperitoneal, or subcutaneous, administration. In some embodiments, the one or more immunosuppressive agents are administered parenterally. The term “parenteral,” as used herein, includes intravenous, intramuscular, subcutaneous, rectal, vaginal, and intraperitoneal administration. In some embodiments, the one or more immunosuppressive agents are administered to a subject using peripheral systemic delivery by intravenous, intraperitoneal, or subcutaneous injection.

Compositions in some embodiments are provided as sterile liquid preparations, e.g. isotonic aqueous solutions, suspensions, emulsions, or dispersions, which may in some aspects be buffered to a selected pH. Liquid compositions are somewhat more convenient to administer, especially by injection. Liquid compositions can comprise carriers, which can be a solvent or dispersing medium containing, for example, water, saline, phosphate buffered saline, polyol (for example, glycerol, propylene glycol, liquid polyethylene glycol) and suitable mixtures thereof. Sterile injectable solutions can be prepared by incorporating the one or more immunosuppressive agents in a solvent, such as in admixture with a suitable carrier, diluent, or excipient such as sterile water, physiological saline, glucose, dextrose, or the like.

In some embodiments, the pharmaceutical composition can be formulated for administration by any route known to those of skill in the art including intramuscular, intravenous, intradermal, intralesional, intraperitoneal injection, subcutaneous, intratumoral, epidural, nasal, oral, vaginal, rectal, topical, local, otic, inhalational, buccal (e.g. sublingual), and transdermal administration or any route. In some embodiments, other modes of administration also are contemplated. In some embodiments, the administration is by bolus infusion, by injection, e.g. intravenous or subcutaneous injections, intraocular injection, periocular injection, subretinal injection, intravitreal injection, trans-septal injection, subscleral injection, intrachoroidal injection, intracameral injection, subconjunctival injection, sub-Tenon's injection, retrobulbar injection, peribulbar injection, or posterior juxtascleral delivery. In some embodiments, administration is by parenteral, intrapulmonary, and intranasal, and, if desired for local treatment, intralesional administration. Parenteral infusions include intramuscular, intravenous, intraarterial, intraperitoneal, or subcutaneous administration. In some embodiments, the administration is via the portal vein. In some embodiments, the administration is by injection into the intramuscular space forearm of the subject.

In some embodiments, compositions also can be administered with other biologically active agents, either sequentially, intermittently or in the same composition. In some embodiments, administration also can include controlled release systems including controlled release formulations and device-controlled release, such as by means of a pump. In some embodiments, the administration is oral. In some embodiments, the administration is intravenous.

In some embodiments, a pharmaceutically acceptable carrier can include all solvents, dispersion media, coatings, antibacterial and antifungal agents, isotonic and absorption delaying agents, and the like, compatible with pharmaceutical administration (Gennaro, 2000, Remington: The science and practice of pharmacy, Lippincott, Williams & Wilkins, Philadelphia, PA). Examples of such carriers or diluents include, but are not limited to, water, saline, Ringer's solutions, dextrose solution, and 5% human serum albumin. Liposomes and non-aqueous vehicles such as fixed oils may also be used. Supplementary active compounds can also be incorporated into the compositions. The pharmaceutical carrier should be one that is suitable for the one or more immunosuppressive agents, such as a saline solution, a dextrose solution or a solution comprising human serum albumin. In some embodiments, the pharmaceutically acceptable carrier or vehicle for such compositions is any non-toxic aqueous solution in which the engineered islets can be maintained, or remain viable, for a time sufficient to allow administration of live cells. For example, the pharmaceutically acceptable carrier or vehicle can be a saline solution or buffered saline solution.

Also provided herein are compositions that are suitable for cryopreserving the engineered islets. In some embodiments, the engineered islets are cryopreserved in a cryopreservation medium. In some embodiments, the cryopreservation medium is a serum free cryopreservation medium. In some embodiments, the composition comprising the engineered islets or population thereof comprises a cryoprotectant. In some embodiments, the cryoprotectant is or comprises DMSO and/or s glycerol. In some embodiments, the cryopreservation medium is between at or about 5% and at or about 10% DMSO (v/v). In some embodiments, the cryopreservation medium is at or about 5% DMSO (v/v). In some embodiments, the cryopreservation medium is at or about 6% DMSO (v/v). In some embodiments, the cryopreservation medium is at or about 7% DMSO (v/v). In some embodiments, the cryopreservation medium is at or about 7.5% DMSO (v/v). In some embodiments, the cryopreservation medium is at or about 8% DMSO (v/v). In some embodiments, the cryopreservation medium is at or about 9% DMSO (v/v). In some embodiments, the cryopreservation medium is at or about 10% DMSO (v/v). In some embodiments, the cryopreservation medium contains a commercially available cryopreservation solution (CryoStor™ CS10). CryoStor™ CS10 is a cryopreservation medium containing 10% dimethyl sulfoxide (DMSO). In some embodiments, compositions formulated for cryopreservation can be stored at low temperatures, such as ultra-low temperatures, for example, storage with temperature ranges from −40° C. to −150° C., such as or about 80° C. 6.0° C.

In some embodiments, the cryopreserved engineered islets are prepared for administration by thawing. In some cases, the engineered islets can be administered to a subject immediately after thawing. In such an embodiment, the composition comprising the engineered islets is ready-to-use without any further processing. In other cases, the engineered islets are further processed after thawing, such as by resuspension with a pharmaceutically acceptable carrier, incubation with an activating or stimulating agent, or are activated washed and resuspended in a pharmaceutically acceptable buffer prior to administration

In some embodiments, the composition, including pharmaceutical composition, is sterile.

In some embodiments, the pharmaceutical composition comprises a engineered islets and a pharmaceutically acceptable carrier comprising 31.25% (v/v) Plasma-Lyte A, 31.25% (v/v) of 5% dextrose/0.45% sodium chloride, 10% dextran 40 (LMD)/5% dextrose, 20% (v/v) of 25% human serum albumin (HSA), and 7.5% (v/v) dimethylsulfoxide (DMSO).

### C. Dosing and Administration

In some embodiments, the engineered islets can be administered by any route known to those of skill in the art including intramuscular, intravenous, intradermal, intralesional, intraperitoneal injection, subcutaneous, kidney capsule, intratumoral, epidural, nasal, oral, vaginal, rectal, topical, local, otic, inhalational, buccal (e.g. sublingual), and transdermal administration or any route. In some embodiments, other modes of administration also are contemplated. In some embodiments, the administration is by bolus infusion, by injection, e.g. intravenous or subcutaneous injections, intraocular injection, periocular injection, subretinal injection, intravitreal injection, trans-septal injection, subscleral injection, intrachoroidal injection, intracameral injection, subconjunctival injection, sub-Tenon's injection, retrobulbar injection, peribulbar injection, or posterior juxtascleral delivery. In some embodiments, administration is by parenteral, intrapulmonary, and intranasal, and, if desired for local treatment, intralesional administration. Parenteral infusions include intramuscular, intravenous, intraarterial, intraperitoneal, or subcutaneous administration. In some embodiments, the administration is via the portal vein. In some embodiments, the administration is by injection into the intramuscular space forearm of the subject. In some embodiments, the administration is by kidney capsule.

In some embodiments, the engineered islets may be administered at any suitable location in the subject. For example, in some embodiments, the engineered islets are administered to the kidney, forearm, mouth, anus, nose, upper arm, hip, thigh, buttocks, liver, spleen, muscle, subcutaneous tissue, or white adipose tissue of the subject. In some embodiments, the engineered cells are administered to the liver, muscle, or white adipose tissue of the subject. In some embodiments, the white adipose tissue is omentum.

In particular embodiments, the engineered islets are administered by intramuscular injection. In some embodiments, the engineered islets are administered to the forearm of the subject. In some embodiments, the engineered islets are administered to the intramuscular space of the forearm of the subject.

In some embodiments, injections into the muscle circumvent early islet loss through an instant blood-mediated inflammatory reaction (IBMIR) that is known to occur after portal vein injections (Bennet et al., Diabetes (1999) 48:1907-1914). The muscle is well vascularized and islet transplantations into striated muscle have been successful clinically (Christoffersson et al, Diabetes (2010) 59:2569-2578; Rafael et al., Am J Transplant (2008) 8:458-462).

In some aspects, the methods of administration involve implanting engineered islets cells into the subject. In some aspects, the engineered islets may be implanted as dispersed cells or formed into clusters. In some embodiments, the engineered islets are administered as a suspension of a population of islet cells. In some embodiments, the engineered islets are an engineered tissue graft comprising a population of engineered islet cells and a matrix. In some embodiments, the engineered islet cells are in a composition that is administered as a suspension of a population of engineered islet cells.

The specific amount/dosage regimen of the engineered islets will vary depending on the weight, gender, age and health of the subject; the formulation, the biochemical nature, bioactivity, bioavailability and the side effects of the engineered islets, and the number and identity of the engineered cells. The dose for administration can depend on a number of various factors including the patient's condition and response to the therapy, and can be determined by one skilled in the art.

In some embodiments, the dose of engineered islets is administered in an amount from or from about 1000 islet equivalent units (IEQ) to at or about 1×106 IEQ, such as from or from about 1000 IEG to at or about 500,000 IEQ, at or about 1000 IEQ to at or about 250,000 IEQ, at or about 1000 IEQ to at or about 100,000 IEQ, at or about 1000 IEQ to at or about 50,000 IEQ, at or about 1000 IEQ to at or about 25,000 IEQ, at or about 1000 IEQ to at or about 10000 IEQ, at or about 1000 IEQ to at or about 5000 IEQ, at or about 5000 IEQ to at or about 1×106 IEQ, at or about 5000 IEQ to at or about 500,000 IEQ, at or about 5000 IEQ to at or about 250,000 IEQ, at or about 5000 IEQ to at or about 100,000 IEQ, at or about 5000 IEQ to at or about 50,000 IEQ, at or about 5000 IEQ to at or about 250000 IEQ, at or about 5000 IEQ to at or about 10000 IEQ, at or about 10000 IEQ to at or about 1×106 IEQ, at or about 10000 IEQ to at or about 500000 IEQ, at or about 10000 IEQ to at or about 250000 IEQ, at or about 10000 IEQ to at or about 100000 IEQ, at or about 10000 IEQ to at or about 50000 IEQ, at or about 10000 IEQ to at or about 250000 IEQ, at or about 25000 IEQ to at or about 1×106 IEQ, at or about 25000 IEQ to at or about 500000 IEQ, at or about 25000 IEQ to at or about 250000 IEQ, at or about 25000 IEQ to at or about 100000 IEQ, at or about 25000 IEQ to at or about 50000 IEQ, at or about 50000 IEQ to at or about 1×106 IEQ, at or about 50000 IEQ to at or about 500000 IEQ, at or about 50000 IEQ to at or about 150000 IEQ, at or about 50000 IEQ to at or about 100000 IEQ, at or about 100000 IEQ to at or about 1×106 IEQ, at or about 100000 IEQ to at or about 500000 IEQ, at or about 100000 IEQ to at or about 250000 IEQ, at or about 250000 IEQ to at or about 1×106 IEQ, at or about 250000 IEQ to at or about 500000 IEQ, or at or about 500000 IEQ to at or about 1×106 IEQ. In some embodiments, the modified SB-beta cells are administered in an amount that is at or about 50,000 IEQ, at or about 100,000 IEQ, at or about 200,000 IEQ, at or about 300,000 IEQ, at or about 400,000 IEQ, or at or about 500,000 IEQ, or any value between any of the foregoing. IEQ provides a standardized estimate of islet volume, with one IEQ corresponding to the volume of a perfectly spherical islet with a diameter of 150 m (Ricordi et al. Acta Diabetol. Lat. 27, 185-195 (1990).

In some embodiments, the dose of engineered islets administered to a subject is administered per kg of body weight of the subject. In some embodiments, the engineered islets are administered in a dosage amount of from at or about 500 IEQ/kg of body weight to at or about 10000 IEQ/kg, from at or about 500 IEQ/kg to at or about 5000 IEQ/kg, from at or about 500 IEQ/kg to at or about 2500 IEQ/kg, from at or about 500 IEQ/kg to at or about 1000 IEQ/kg, from at or about 1000 IEQ/kg to at or about 10000 IEQ/kg, from at or about 1000 IEQ/kg to at or about 5000 IEQ/kg, from at or about 1000 IEQ/kg to at or about 2500 IEQ/kg, from at or about 2500 IEQ/kg to at or about 10000 IEQ/kg, from at or about 2500 IEQ/kg to at or about 5000 IEQ/kg, or from at or about 5000 IEQ/kg to at or about 10000 IEQ/kg.

Any therapeutically effective amount of cells described herein can be included in the pharmaceutical composition, depending on the indication being treated. Non-limiting examples of the cells include primary islet cells (e.g. engineered hypoimmunogenic islet cells) as described. In some embodiments, the pharmaceutical composition includes at least about 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸ cells. In some embodiments, the pharmaceutical composition includes up to about 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸ cells. In some embodiments, the pharmaceutical composition includes up to about 1×10⁷ cells. In some embodiments, the pharmaceutical composition includes up to about 3×10⁷ cells. In some embodiments, the pharmaceutical composition includes at least about 1×10⁷-3×10⁷, 2×10⁷-4×10⁷, 3×10⁷-5×10⁷, 4×10⁷-6×10⁷, 5×10⁷-7×10⁷, 6×10⁷-8×10⁷, 7×10⁷-9×10⁷, 8×10⁷-1×10⁸, 9×10⁷-2×10⁸, or 1×10⁸-3×10⁸ cells. In exemplary embodiments, the pharmaceutical composition includes from about 1×10⁷ to about 3×10⁷ cells. In some embodiments, the pharmaceutical composition includes at least about 25×10⁶ to at least about 25×10⁷ cells. In some embodiments, the pharmaceutical composition includes at least about 80×10⁶ to at least about 80×10⁷ cells. In another exemplary embodiment, the pharmaceutical composition includes about 25×10⁶ to about 80×10⁶ cells. In some embodiments, the pharmaceutical composition includes from about 25×10⁶ to about 80×10⁷ cells.

In some embodiments, the pharmaceutical composition is administered as a single dose of from about 1.25×10⁷ to about 1.2×10⁷ engineered hypoimmunogenic islet cells per kg body weight. In some embodiments, the pharmaceutical composition is administered as a single dose of from about 1.25×10⁷ to about 1.25×10⁶, about 1.5×10⁷ to about 1.5×10⁶, about 2.0×10⁷ to about 2.0×10⁶, about 2.5×10⁷ to about 2.5×10⁶, about 3.0×10⁷ to about 3.0×10⁶, about 3.5×10¹ to about 3.5×10⁶, about 4.0×10⁷ to about 4.0×10⁶, about 4.5×10⁷ to about 4.5×10⁶, about 5.0×10¹ to about 5.0×10⁶, about 5.5×10⁷ to about 5.5×10⁶, about 6.0×10⁷ to about 6.0×10⁶, about 6.5×10¹ to about 6.5×10⁶, about 7.0×10⁷ to about 7.0×10⁶, about 7.5×10⁷ to about 7.5×10⁶, about 8.0×10⁷ to about 8.0×10⁶, about 8.5×10⁷ to about 8.5×10⁶, about 9.0×10⁷ to about 9.0×10⁶, about 1.0×10⁶ to about 1.0×10⁷, or about 1.2×10⁶ to about 1.2×10⁷ cells per kg body weight. In many embodiments, the dose is at a range that is lower than from about 1.25×10⁵ to about 1.2×10⁷ cells per kg body weight. In many embodiments, the dose is at a range that is higher than from about 1.25×10⁵ to about 1.2×10⁷ cells per kg body weight. In some embodiments, the dose is administered intravenously.

In some embodiments, the pharmaceutical composition includes islet equivalents (IEQ). In some embodiments, the pharmaceutical composition includes at least about 6,500 IEQ, 50,000 IEQ, 100,500 IEQ, 200,000 IEQ, 300,000 IEQ, 400,000 IEQ, 500,000 IEQ, or 600,000 IEQ. In some embodiments, the pharmaceutical composition includes up to about 6,500 IEQ, 50,000 IEQ, 100,500 IEQ, 200,000 IEQ, 300,000 IEQ, 400,000 IEQ, 500,000 IEQ, or 600,000 IEQ. In some embodiments, the pharmaceutical composition includes up to about 6,500 IEQ. In some embodiments, the pharmaceutical composition includes up to about 600,000 IEQ. In some embodiments, the pharmaceutical composition includes at least about 6,500 IEQ, 50,000 IEQ, 100,500 IEQ, 200,000 IEQ, 300,000 IEQ, 400,000 IEQ, 500,000 IEQ, or 600,000 IEQ. In exemplary embodiments, the pharmaceutical composition includes from about 6,500 to about 600,000 IEQ.

In some embodiments, the pharmaceutical composition is administered as a single dose of from about 80 IEQ/kg to about 24,000 IEQ/kg. In some embodiments, the pharmaceutical composition is administered as a single dose of from about 80 IEQ/kg to about 800 IEQ/kg, about 100 IEQ/kg to about 1,000 IEQ/kg, about 200 IEQ/kg to about 2,000 IEQ/kg, about 300 IEQ/kg to about 3,000 IEQ/kg, about 400 IEQ/kg to about 4000 IEQ/kg, about 500 IEQ/kg to about 5,000 IEQ/kg, about 1,000 IEQ/kg to about 10,000 IEQ/kg, about 5,000 IEQ/kg to about 15,000 IEQ/kg, about 10,000 IEQ/kg to about 20,000 IEQ/kg, or about 14,000 IEQ/kg to about 24,000 IEQ/kg. In many embodiments, the dose is at a range that is lower than from about 80 IEQ/kg to about 24,000 IEQ/kg. In many embodiments, the dose is at a range that is higher than from about 80 IEQ/kg to about 24,000 IEQ/kg. In some embodiments, the dose is administered intravenously.

In some embodiments, the pharmaceutical composition is administered as a single dose of from about 500 to about 1500 islets per cluster. In some embodiments, the pharmaceutical composition is administered as a single dose of from about 500, 1000, or 1500 islets per cluster.

### D. Subjects

### 1. Beta Cell Related Disorders

The modified cells provided herein can be administered to any suitable subjects (e.g. patients) including, for example, a candidate for a cellular therapy for the treatment of a beta cell related disease or disorder. Candidates for cellular therapy include any subject having a beta cell related disease or disorder that may potentially benefit from the therapeutic effects of the subject modified beta cells and one or more immunosuppressive agents provided herein. In some embodiments, the subject is an allogenic recipient of the administered modified beta cells. In some embodiments, the provided modified beta cells and one or more immunosuppressive agents are effective for use in allogeneic cell therapy. A subject who benefits from the therapeutic effects of the subject modified beta cells and one or more immunosuppressive agents provided herein exhibit an elimination, reduction, or amelioration of the beta cell related disease or disorder. In some aspects, the subject has, or has an increased risk of developing, a beta cell related disorder.

In some embodiments, the beta cell related disorder is a metabolic disorder. A metabolic disorder may occur when abnormal chemical reactions in the body of a subject disrupts metabolic processes (e.g. processes related to the metabolism, or breakdown, of energy into sugars and acids or the storage of said energy). In some embodiments, the metabolic disorder affects the breakdown of amino acids, carbohydrates, or lipids in a subject's body. In some embodiments, the metabolic disorder affects the subject's mitochondria (e.g. mitochondrial diseases). In some embodiments, the metabolic disorder develops when the subject's organs, such as the liver or pancreas, become disease and/or do not function normally. Exemplary metabolic disorders herein may comprise, but are not limited to, any disease or disorder characterized by increased blood pressure, high blood sugar, excess body fat around the waist, and abnormal cholesterol or triglyceride levels. In some embodiments, the metabolic disorder is familial hypercholesterolemia, Gaucher disease, Hunter syndrome, Krabbe disease, maple syrup urine disease, metachromatic leukodystrophy, mitochondrial encephalopathy, lactic acidosis, stroke-like episodes (MELAS), Niemann-Pick disease, phenylketonuria (PKU), porphyria, Tay-Sachs disease, Wilson's disease, Type I diabetes, Type II diabetes, obesity, hypertension, dyslipidemia, or carbohydrate intolerance. In some embodiments, the metabolic disorder is Type II diabetes. In some embodiments, the metabolic disorder is Type I diabetes. In some embodiments, the metabolic disorder is Type I diabetes mellitus.

In some embodiments, the subject has been diagnosed with the beta cell related disease or disorder (e.g. Type I diabetes) prior to the administration of the one or more immunosuppressive agents and/or the modified beta cell or composition, such as any of the immunosuppressive agents and/or the compositions comprising a modified beta cell described herein. In some embodiments, the subject has been diagnosed with the beta cell related disease or disorder between about 1 year and about 5 years prior to the administration of the one or more immunosuppressive agents and/or the modified beta cell or composition. In some embodiments, the subject has been diagnosed with the beta cell related disease or disorder at least about 1 year prior to the administration of the one or more immunosuppressive agents and/or the modified beta cell or composition, such as at least about any of 2 years, 3 years, 4 years, 5 years, or more, prior to the administration of the one or more immunosuppressive agents and/or the modified beta cell or composition. In some embodiments, the subject has been diagnosed with the beta cell related disease or disorder less than about 5 years prior to the administration of the one or more immunosuppressive agents and/or the modified beta cell or composition, such as less than about any of 4 years, 3 years, 2 years, 1 year, or less, prior to the administration of the one or more immunosuppressive agents and/or the modified beta cell or composition. In some embodiments, the subject has been diagnosed with Type I diabetes at least about 1 year prior to the administration of the one or more immunosuppressive agents and/or the modified beta cell or composition, such as at least about any of 2 years, 3 years, 4 years, 5 years, or more, prior to the administration of the one or more immunosuppressive agents and/or the modified beta cell or composition. In some embodiments, the subject has been diagnosed with Type I diabetes less than about 5 years prior to the administration of the one or more immunosuppressive agents and/or the modified beta cell or composition, such as less than about any of 4 years, 3 years, 2 years, 1 year, or less, prior to the administration of the one or more immunosuppressive agents and/or the modified beta cell or composition.

### 2. Inclusion Criteria

In some embodiments, the subject displays one or more inclusion criteria prior to administration of the dose of engineered hypoimmunogenic islets. The term “inclusion criteria” as used herein refers to clinical phenotypes of the subject that qualify said subject for application of the methods and uses provided herein.

In some embodiments, the subject is a juvenile, a teenager, middle aged, or elderly. In some embodiments, the subject is a juvenile. In some embodiments, the subject is between the ages of about 1 month old and about 18 years old, such as between about 1 month and about 1 year, between about 6 months and about 5 years, between about 2 years and about 10 years, or between about 8 years and about 15 years. In some embodiments, the subject is older than about 1 month old, such as older than any of about 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, 10 years, 11 years, 12 years, 13 years, 14 years, 15 years, 16 years, 17 years 18 years old, or older. In some embodiments, the subject is younger than about 18 years old, such as younger than any of about 17 years, 16 years, 15 years, 14 years, 13 years, 12 years, 11 years, 10 years, 9 years, 8 years, 7 years, 6 years, 5 years, 4 years, 3 years, 2 years, 1 year, 11 months, 10 months, 9 months, 8 months, 7 months, 6 months, 5 months, 4 months, 3 months, 2 months, 1 month old or younger. In some embodiments, the subject is between the ages of about 18 years old to about 90 years old, such as between about 18 years old and about 40 years old, between about 20 years old and about 60 years old, between about 50 years old and about 80 years old, or between about 60 years old and about 90 years old. In some embodiments, the subject is older than about 18 years old, such as older than about any of 20 years old, 25 years old, 30 years old, 35 years old, 40 years old, 45 years old, 50 years old, 55 years old, 60 years old, 65 years old, 70 years old, 75 years old, 80 years old, 85 years old, 90 years old, or older. In some embodiments, the subject is younger than about 90 years old, such as younger than about any of 85 years old, 80 years old, 75 years old, 70 years old, 65 years old, 60 years old, 55 years old, 50 years old, 45 years old, 40 years old, 35 years old, 30 years old, 25 years old, 20 years old, 18 years old, or younger.

In some embodiments, the subject to be treated is characterized by one or more of the following: diagnosed before the age of 18 years; involved in intensive diabetes management; between the ages of 18-45; and body weight <80 kg. In some embodiments, the subject to be treated is diagnosed before the age of 18 years. In some embodiments, the subject to be treated is involved in intensive diabetes management. In some embodiments, the intensive diabetes management comprises self-monitoring of subcutaneous glucose level by continuous glucose monitoring or by intermittent scanning glucose monitoring no less than a mean of three times per day averaged over each week. In some embodiments, intensive diabetes management comprises administration of three or more insulin injections per day or insulin pump therapy. In some embodiments, the intensive diabetes management comprises self-monitoring of subcutaneous glucose level by continuous glucose monitoring or by intermittent scanning glucose monitoring no less than a mean of three times per day averaged over each week and administration of three or more insulin injections per day or insulin pump therapy. In some embodiments, the subject to be treated is between the ages of 18-45. In some embodiments, the subject to be treated is <80 kg.

### 3. Exclusion Criteria

In some embodiments, the subject does not display any one of exclusion criteria prior to the administration of the dose of engineered hypoimmunogenic islets. The term “exclusion criteria” as used herein refers to clinical phenotypes of the subject that disqualify said subject for application of the methods and uses provided herein.

In some embodiments, the subject is not characterized by having the following: any previous organ transplantation; any history of malignancy; use of any investigational agent(s) within 4 weeks of administering the dose of engineered hypoimmunogenic islets; use of any anti-diabetic medication other than insulin within 4 weeks of administering the dose of engineered hypoimmunogenic islets; active infections including Tuberculosis, HIV, HBV and HCV; liver function test value for AST, ALT, GGT or ALP exceeding the respective reference interval; serological evidence of infection with HTLVI or HTLVII; pregnancy, nursing, intention for pregnancy; chronic kidney disease grade 3 or worse (GFR <60 ml/min as estimated by creatine measurement); medical history of cardiac disease or symptoms at screening consistent with cardiac disease; HLA immunization, MIC A/B immunization; known autoimmune disease other than type I diabetes (e.g., Hashimoto disease); administration of live attenuated vaccines <6 months before administering the dose of engineered hypoimmunogenic islets; islet antibodies GADA >2000 IE/mL or IA2A >4000 IE/mL or ZnT8 autoantibodies; untreated proliferative diabetic retinopathy; ongoing psychiatric illness; ongoing substance abuse, drug or alcohol or treatment noncompliance; and known hypersensitivity to ciprofloxacin, gentamicin, or amphotericin.

In some embodiments, the subject has not had any previous organ transplantation. In some embodiments, the subject has not had any history of malignancy. In some embodiments, the subject has not used any investigational agent(s) within 4 weeks of receiving the dose of engineered hypoimmunogenic islets. In some embodiments, the subject has not used any anti-diabetic medication other than insulin within 4 weeks of receiving the dose of engineered hypoimmunogenic islets. In some embodiments, the subject has not had any active infections including Tuberculosis, HIV, HBV and HCV. In some embodiments, the subject has not had a liver function test value for AST, ALT, GGT or ALP exceeding the respective reference interval. In some embodiments, the subject has not had serological evidence of infection with HTLVI or HTLVII. In some embodiments, the subject is not pregnant, nursing or intending to be pregnant. In some embodiments, the subject does not have chronic kidney disease grade 3 or worse (GFR <60 ml/min as estimated by creatine measurement). In some embodiments, the subject does not have any medical history of cardiac disease or symptoms at screening consistent with cardiac disease. In some embodiments, the subject has not had HLA immunization or MIC A/B immunization. In some embodiments, the subject does not have any known autoimmune disease other than type I diabetes (e.g., Hashimoto disease). In some embodiments, the subject has not received administration of live attenuated vaccines <6 months before receiving the dose of engineered hypoimmunogenic islets. In some embodiments, the subject does not have islet antibodies GADA >2000 IE/mL, IA2A >4000 IE/mL, or ZnT8 autoantibodies. In some embodiments, the subject does not have untreated proliferative diabetic retinopathy. In some embodiments, the subject does not have ongoing psychiatric illness. In some embodiments, the subject does not have ongoing substance abuse, drug or alcohol or treatment noncompliance. In some embodiments, the subject does not have known hypersensitivity to ciprofloxacin, gentamicin, or amphotericin.

### E. Outcomes of the Method

Provided herein are methods relating to administering to a subject engineered islets, generally including engineered beta islet cells. In some embodiments, the provided methods are useful for treating a beta cell related disorder (e.g., Type I diabetes) in a subject, promoting engraftment or survival of a beta cell in a subject, and/or restoring glucose metabolism in a subject.

In some embodiments, the provided methods may improve glucose tolerance in a subject. Glucose tolerance may be measured by any suitable method, such as those described herein (e.g. insulin secretion assays). In some embodiments, the engineered islets exhibits glucose-stimulated insulin secretion (GSIS). Thus, in some embodiments, the improved glucose tolerance is measured in a GSIS perfusion assay. Glucose intolerance is related to insulin resistance, and can cause diabetes (e.g. Type 1 diabetes and Type II diabetes). Therefore, in some embodiments, provided is a method of treating a beta cell related disorder (e.g. diabetes) comprising administering provided engineered islets to a subject. In some embodiments, the subject is a diabetic patient. In some embodiments, the subject has Type I diabetes. In some embodiments, the subject has Type II diabetes. Specifically, in some embodiments, provided is a method of improving glucose tolerance in a subject, the method comprising administering engineered islets as described herein to a subject. In some embodiments, glucose tolerance is improved relative to the subject's glucose tolerance prior to administration of the engineered islets. In some embodiments, the engineered islets reduce exogenous insulin usage in the subject. In some embodiments, glucose tolerance is improved as measured by HbA1c levels. In some embodiments, the subject is fasting. In some embodiments, the engineered islets improve insulin secretion in the subject. In some embodiments, insulin secretion is improved relative to the subject's insulin secretion prior to administration of the engineered islets.

In some embodiments, the methods disclosed herein further include monitoring a patient for insulin-independence. In some embodiments, “insulin-independence” or “insulin-independent” is achieved in a subject (e.g., an islet cell recipient) that is able to titrate off insulin therapy for at least 1 week and meets one or more, e.g., all, of the following criteria: (i) fasting capillary glucose level does not exceed 140 mg/dL (7.8 mmol/L) more than three times in 1 week (based on measuring capillary glucose levels a minimum of 7 times in a seven day period); (ii) 2-hours post-prandial capillary glucose does not exceed 180 mg/dL (10.0 mmol/L) more than three times in 1 week (based on measuring capillary glucose levels a minimum of 21 times in a seven day period); and (iii) evidence of endogenous insulin production defined as fasting or stimulated C-peptide levels >0.5 ng/mL (0.16 pmol/L). In some embodiments, the subject is characterized by one of (i)-(iii). In some embodiments, the subject is characterized by two of (i)-(iii). In some embodiments, the subject is characterized by each of (i)-(iii).

In some embodiments, the subject is monitored at about 1 month, 2 month, 3 month, 4 month, 5 month, 6 month, 7 month, 8 month, 9 month, 10 month, 11 month, or 12 or more months after administration of any of the cells provided herein (e.g., a dose of engineered hypoimmunogenic islet cells). In some embodiments, the methods disclosed herein include monitoring a subject for up to one year for insulin-independence after administration of any of the cells provided herein (e.g., a dose of engineered hypoimmunogenic islet cells).

In some embodiments, the subject has reduced insulin dependence (e.g. dose of exogenous insulin is reduced by 10% or more, such as 20%, 30%, 40%, 50%, 60%, 70%, 80%, 90% or more), compared to the amount of exogenous insulin required for a subject administered non-hypoimmunogenic islets for treating the beta cell disorder or the amount of exogenous insulin required for untreated subjects that have the beta cell disorder. In some embodiments, the reduce insulin dependence is achieved for 1 month, 2 month, 3 month, 4 month, 5 month, 6 month, 7 month, 8 month, 9 month, 10 month, 11 month, or 12 or more months after administration of any of the cells provided herein (e.g., a dose of engineered hypoimmunogenic islet cells).

In some embodiments, the subject is insulin-independent. In some embodiments, the insulin independence is achieved for 1 month, 2 month, 3 month, 4 month, 5 month, 6 month, 7 month, 8 month, 9 month, 10 month, 11 month, or 12 or more months after administration of any of the cells provided herein (e.g., a dose of engineered hypoimmunogenic islet cells).

In some embodiments, the methods disclosed herein further comprise monitoring the patient one or more times or continuously throughout the period for transplantation survival. In some embodiments, the period of graft survival may be about 1, 2, 3, 4, 5, 6, 7, 8, 9, or 10 years or more (e.g., about 1 year or more, about 2 years or more, about 5 years or more, about 7 years or more, or about 10 years or more).

In some embodiments, the methods disclosed herein further comprise administering one or more additional doses of engineered islets to a subject who, at the end of the monitoring period, is not insulin-independent or insulin-dependent. In some embodiments, the subject is “insulin-dependent” if the subject (e.g., an islet cell recipient) that does not meet the criteria for insulin-independence, as described above. In some embodiments, the methods disclosed herein further comprise administering one or more additional doses of cells to a subject who, at the end of the monitoring period, has a C-peptide level in a serum sample of less than about 0.2, 0.3, 0.4, or 0.5 ng/ml (e.g., about 0.3 ng/ml). In some embodiments, a subject having a C-peptide level in a serum sample of less than about 0.2, 0.3, 0.4, or 0.5 ng/ml (e.g., about 0.3 ng/ml) is not insulin-independent.

In some embodiments, administration of the provided engineered islet cells do not induce and adaptive immune response in the subject. In some embodiments, the adaptive immune response is assessed using ELISPOT. For example, the adaptive immune response may be assessed by measuring the levels of IFNg cytokine secretion by CD8+ T cells. In some embodiments, the levels of IFNg produced following administration of engineered islets is lower than wild type primary islet cells or compared to SC-derived islets cells derived from unmodified pluripotent stem cells, such as by about 400-fold, 300-fold, 200-fold, 100-fold, 50-fold, 25-fold, or 10-fold lower levels of IFNg. In some embodiments, the adaptive immune response is assessed using flow cytometry. For example, in some embodiments, the adaptive immune response is assessed by measuring the levels donor specific antibody (DSA) IgG or IgM. In some embodiments, the engineered islets exhibit lower levels of DSA levels compared to wild type primary islet cells, such as any of about 2-fold, 1.5-fold, and 1-fold lower levels of DSA compared to a control or wild-type beta cells.

In some embodiments, the engineered islet cells are hypoimmunogenic and exhibit a reduced or lower immune response compared to islets cells that are not engineered with the modifications. In some embodiments, an immune response against the engineered cells is reduced or lower by at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of the immune response produced by the administration of immunogenic cells (e.g. a population of cells of the same or similar cell type or phenotype but that do not contain the modifications, e.g. genetic modifications, of the modified cells). In some embodiments, the administered engineered islets fails to elicit an immune response against the modified cells in the subject.

In some embodiments, the administered engineered islets elicits a decreased or lower level of systemic TH1 activation in the subject. In some instances, the level of systemic TH1 activation elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of systemic TH1 activation produced by the administration of immunogenic cells (e.g. a population of cells of the same or similar cell type or phenotype but that do not contain the modifications, e.g. genetic modifications, of the modified cells). In some embodiments, the administered engineered islets fails to elicit systemic TH1 activation in the subject.

In some embodiments, the administered engineered islets elicits a decreased or lower level of immune activation of peripheral blood mononuclear cells (PBMCs) in the subject. In some instances, the level of immune activation of PBMCs elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of immune activation of PBMCs produced by the administration of immunogenic cells (e.g. a population of cells of the same or similar cell type or phenotype but that do not contain the modifications, e.g. genetic modifications, of the modified cells). In some embodiments, the administered engineered islets fails to elicit immune activation of PBMCs in the subject.

In some embodiments, the administered engineered islets elicits a decreased or lower level of donor-specific IgG antibodies in the subject. In some instances, the level of donor-specific IgG antibodies elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of donor-specific IgG antibodies produced by the administration of immunogenic cells (e.g. a population of cells of the same or similar cell type or phenotype but that do not contain the modifications, e.g. genetic modifications, of the modified cells). In some embodiments, the administered population of modified cells fails to elicit donor-specific IgG antibodies in the subject.

In some embodiments, the administered engineered islets elicits a decreased or lower level of IgM and IgG antibody production in the subject. In some instances, the level of IgM and IgG antibody production elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of IgM and IgG antibody production produced by the administration of immunogenic cells (e.g. a population of cells of the same or similar cell type or phenotype but that do not contain the modifications, e.g. genetic modifications, of the modified cells). In some embodiments, the administered engineered islets fails to elicit IgM and IgG antibody production in the subject.

In some embodiments, the administered engineered islets elicits a decreased or lower level of cytotoxic T cell killing in the subject. In some instances, the level of cytotoxic T cell killing elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of cytotoxic T cell killing produced by the administration of immunogenic cells (e.g. a population of cells of the same or similar cell type or phenotype but that do not contain the modifications, e.g. genetic modifications, of the modified cells). In some embodiments, the administered engineered islets fails to elicit cytotoxic T cell killing in the subject.

Upon administration of engineered islets described herein the subject exhibits no systemic immune response or a reduced level of systemic immune response compared to responses to cells that are not hypoimmunogenic. In some embodiments, the subject exhibits no adaptive immune response or a reduced level of adaptive immune response compared to responses to cells that are not hypoimmunogenic. In some embodiments, the subject exhibits no innate immune response or a reduced level of innate immune response compared to responses to cells that are not hypoimmunogenic. In some embodiments, the subject exhibits no T cell response or a reduced level of T cell response compared to responses to cells that are not hypoimmunogenic. In some embodiments, the subject exhibits no B cell response or a reduced level of B cell response compared to responses to cells that are not hypoimmunogenic.

In some embodiments, upon administration of the engineered islets as described herein the subject does not experience any adverse events. In some embodiments, the subject experiences fewer adverse events compared to a subject that is not administered the one or more immunosuppressive agents. In some embodiments, the adverse events are assessed by Common Terminology Criteria for Adverse Events (CTCAE) v5.0. An adverse event may include, but is not limited to, hypo- and hyper-glycemia limits for blood glucose related risks, muscle pain during the administration of the engineered islets local hemorrhage during the administration of the engineered islets and/or the one or more immunosuppressive agents, and/or cytokine release syndrome.

In some embodiments, the administered engineered islets evade the subject's immune system as evaluated by PBMC and serum. In some embodiments, the engineered islets evade the subject's immune system at 0, 2, 4, 8, 12, 18, 26, and 52 weeks following administration of the engineered islets to the subject. In some embodiments, the administered engineered islets survive in the subject as evaluated by MRI. In some embodiments, the engineered islets survive within 48 hours following administration of the engineered islets to the subject. In some embodiments, the engineered islets survive 2, 4, 6, 8, 12, 26, and 52 weeks following administration of the engineered islets to the subject. In some embodiments, upon administration of the engineered islets, the subject exhibits a peak c-peptide that is >0.01 nmol/1 in response to a mixed meal tolerance test (MMTT). In some embodiments, the peak c-peptide is >0.01 nmol/1 in response to a MMTT 4, 8, 12, 18, 26, and 52 weeks following administration of the engineered islets to the subject. In some embodiments, the peak c-peptide is measured by area under the curve (AUC). In some embodiments, upon administration of the engineered islets, the subject exhibits a non-fasting c-peptide concentration that is >0.01 nmol/l. In some embodiments, the non-fasting c-peptide concentration is >0.01 nmol/l at 1, 2, 3, 4, 6, 8, 10, 12, 14, 16, 18, 20, 22, 24, 26, and 52 weeks following administration of the engineered islets to the subject. In some embodiments, upon administration of the engineered islets, the subject exhibits decreased insulin requirement per kilogram of body weight (BW). In some embodiments, the insulin requirement per kilogram of BW decreases 1, 2, 3, 4, 6, 8, 10, 12, 14, 16, 18, 20, 22, 24, 26, and 52 weeks following administration of the engineered islets to the subject. In some embodiments, upon administration of the engineered islets, the subject exhibits decreased HbA1c. In some embodiments, the HbA1c decrease 2, 4, 6, 8, 10, 12, 14, 16, 18, 20, 26, and 52 weeks following administration of the engineered islets to the subject. In some embodiments, upon administration of the engineered islets, the subject exhibits reductions in glucose variability. In some embodiments, glucose variability is reduced at 4, 8, 12, 18, 26, and 52 weeks following administration of the engineered islets to the subject. In some embodiments, upon administration of the engineered islets, the subject exhibits reductions in hypoglycemia. In some embodiments, hypoglycemia is reduced at 4, 8, 12, 18, 26, and 52 weeks following administration of the engineered islets to the subject. In some embodiments, upon administration of the engineered islets, the subject exhibits reductions in hyperglycemia. In some embodiments, hyperglycemia is reduced at 4, 8, 12, 18, 26, and 52 weeks following administration of the engineered islets to the subject.

### II. Methods and Dosing of a Beta Cell Therapy in Combination with Immunosuppressive Agents

Provided herein are methods and uses for a combination therapy comprising an engineered islet, such as dose of engineered hypoimmunogenic islets, and one or more immunosuppressive agents.

### A. Immunosuppressive Agents and Regimens Thereof

In some aspects of the methods, combinations, kits, and uses provided herein, one or more immunosuppressive agents are administered to a subject. In some embodiments, the goal of immunosuppression may include promoting engraftment and/or promoting survival of the modified beta cell or composition (e.g., composition comprising modified beta cells) in a subject, while simultaneously minimizing drug toxicities, infection, and malignancy in the subject. In some embodiments, the one or more immunosuppressive agents are administered to the subject in combination with a composition comprising a modified beta cell for use in methods of treating beta cell related disorders, including diabetes (e.g., Type I diabetes). In some embodiments, the provided methods of administering one or more immunosuppressive agents and a composition comprising a modified beta cell are useful for restoring or providing glucose metabolism to a subject in need thereof.

### 1. Administration

In some embodiments, the provided methods involve administering to the subject one or more immunosuppressive agents and a composition comprising a modified beta cell.

In some embodiments, the provided methods involve administration of at least one regimen of one or more immunosuppressive agents prior to, after, during, during the course of, concurrent with, sequentially with, and/or intermittently with administration of the modified beta cell or composition. In some embodiments, the provided methods involve administration of a first dose of one or more immunosuppressive agents prior to, subsequent to (after), during, during the course of, concurrent with, sequentially with, or intermittently with administration of the modified beta cell or composition. In some embodiments, “concurrently” indicates that the administration of one or more immunosuppressive agents and that of the modified beta cell or composition overlap with each other, in that at least one regimen of one or more immunosuppressive agents overlaps with administration of the composition comprising a modified beta, and/or that the administration of one or more immunosuppressive agents occurs at the same time (e.g. same day and/or simultaneously) as administration of the modified beta cell or composition.

In some embodiments, the methods comprise administering one or more immunosuppressive agents (e.g. one or more regimens of one or more immunosuppressive agents) prior to, concurrent with, and/or after administration of the modified beta cell or composition to the subject.

In some embodiments, administering one or more immunosuppressive agents comprises administering at least one regimen of one or more immunosuppressive agents prior to administration of the modified beta cell or composition to the subject. In some embodiments, the one or more immunosuppressive agents are administered to the subject only prior to administration of a first and/or second regimen of the modified beta cell or composition. In some embodiments, the one or more immunosuppressive agents are administered between about 30 seconds and about 10 weeks prior to administration of the modified beta cell or composition to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks prior to administration of the modified beta cell or composition to the subject. In some embodiments, the one or more immunosuppressive agents are administered at least about 30 seconds prior to administration of the modified beta cell or composition to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, or more, prior to administration of the modified beta cell or composition to the subject. In some embodiments, the one or more immunosuppressive agents are administered less than about 10 weeks prior to administration of the modified beta cell or composition to the subject, such less than about any of 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the modified beta cell or composition to the subject. In some embodiments, the one or more immunosuppressive agents are administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the modified beta cell or composition to the subject. In some embodiments, the one or more immunosuppressive agents are administered to the subject prior to administration of the modified beta cell or composition, and continued to be administered over the course of the subject's lifespan. In some embodiments, the one or more immunosuppressive agents are administered to the subject prior to each round of administration of the modified beta cell or composition. In some embodiments, the one or more immunosuppressive agents are administered to the subject prior to each round of administration of the modified beta cell or composition, and continued to be administered over the course of the subject's lifespan.

In some embodiments, administering one or more immunosuppressive agents comprises administering one or more immunosuppressive agents on the same day as the administration of the modified beta cell or composition to the subject. In some embodiments, administering one or more immunosuppressive agents comprises administering one or more immunosuppressive agents concurrent with administration of the modified beta cell or composition to the subject. In some embodiments, administering one or more immunosuppressive agents comprises administering one or more immunosuppressive agents on the same day as administration of the modified beta cell or composition to the subject, and continued to be administered over the course of the subject's lifespan. In some embodiments, administering one or more immunosuppressive agents comprises administering one or more immunosuppressive agents concurrent with administration of the modified beta cell or composition to the subject, and continued to be administered over the course of the subject's lifespan. In some embodiments, a first regimen of one or more immunosuppressive agents is administered to the subject concurrent with administration of the modified beta cell or composition to the subject. In some embodiments, a second regimen of one or more immunosuppressive agents is administered to the subject concurrent with administration of the modified beta cell or composition to the subject. In some embodiments, the one or more immunosuppressive agents are administered to the subject on the same day as each round of administration of the modified beta cell or composition. In some embodiments, the one or more immunosuppressive agents are administered to the subject on the same day as each round of administration of the modified beta cell or composition, and continued to be administered over the course of the subject's lifespan. In some embodiments, the one or more immunosuppressive agents are administered to the subject concurrent with each round of administration of the modified beta cell or composition. In some embodiments, the one or more immunosuppressive agents are administered to the subject concurrent with each round of administration of the modified beta cell or composition, and continued to be administered over the course of the subject's lifespan.

In some embodiments, administering one or more immunosuppressive agents comprises administering a regimen (e.g. at least one regimen) of one or more immunosuppressive agents after administration of the modified beta cell or composition to the subject. In some embodiments, the one or more immunosuppressive agents are administered to the subject only after administration of a first and/or second regimen of the modified beta cell or composition. In some embodiments, the one or more immunosuppressive agents are administered between about 30 seconds and about 10 weeks after administration of the modified beta cell or composition to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks after administration of the modified beta cell or composition to the subject. In some embodiments, the one or more immunosuppressive agents are administered at least about 30 seconds after administration of the modified beta cell or composition to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, or more, after administration of the modified beta cell or composition to the subject. In some embodiments, the one or more immunosuppressive agents are administered less than about 10 weeks after administration of the modified beta cell or composition to the subject, such less than about any of 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the modified beta cell or composition to the subject. In some embodiments, the one or more immunosuppressive agents are administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds after administration of the modified beta cell or composition to the subject. In some embodiments, the one or more immunosuppressive agents are administered to the subject after administration of the modified beta cell or composition, and continued to be administered over the course of the subject's lifespan. In some embodiments, the one or more immunosuppressive agents are administered to the subject after each round of administration of the modified beta cell or composition. In some embodiments, the one or more immunosuppressive agents are administered to the subject after each round of administration of the modified beta cell or composition, and continued to be administered over the course of the subject's lifespan.

In some embodiments, the one or more immunosuppressive agents are administered to the subject at a lower dosage compared to the dosage of one or more immunosuppressive agents administered the subject to reduce immune rejection of immunogenic cells that do not comprise the modifications of the modified beta cell.

In some embodiments, the one or more immunosuppressive agents are administered to the subject in a single regimen (e.g. dose). In some embodiments, the one or more immunosuppressive agents are administered to the subject in plurality of regimens. In some embodiments, the one or more immunosuppressive agents are administered daily. In some embodiments, the one or more immunosuppressive agents are administered at least once daily. In some embodiments, the total daily dosage of the one or more immunosuppressive agents is provided as a single regimen per day. In some embodiments, the one or more immunosuppressive agents are administered as a divided regimen.

In some embodiments, the total daily dosage of the one or more immunosuppressive agents is divided between 2 regimens, 3 regimens, or 4 regimens per day. In some embodiments, the total daily dosage of the one or more immunosuppressive agents is divided between 2 regimens per day. In some embodiments, a regimen of the one or more immunosuppressive agents is administered about every 12 hours. In some embodiments, the total daily dosage of the one or more immunosuppressive agents is divided between 3 regimens per day. In some embodiments, the total daily dosage of the one or more immunosuppressive agents is divided between 4 regimens per day.

In some embodiments, the one or more immunosuppressive agents are administered (e.g. administered daily) for about 3 months, about 6 months, about 12 months, about 24 months, about 36 months, about 48 months, about 60 months, or more after administration of the modified beta cell or composition to the subject. In some embodiments, the one or more immunosuppressive agents are administered for about 3 months after administration of the modified beta cell or composition to the subject. In some embodiments, the one or more immunosuppressive agents are administered for about 6 months after administration of the modified beta cell or composition to the subject. In some embodiments, the one or more immunosuppressive agents are administered for about 9 months after administration of the modified beta cell or composition to the subject. In some embodiments, the one or more immunosuppressive agents are administered for about 12 months after administration of the modified beta cell or composition to the subject. In some embodiments, the one or more immunosuppressive agents are administered for about 24 months after administration of the modified beta cell or composition to the subject. In some embodiments, the one or more immunosuppressive agents are administered for about 48 months after administration of the modified beta cell or composition to the subject. In some embodiments, the one or more immunosuppressive agents are administered for about 60 months after administration of the modified beta cell or composition to the subject.

In some embodiments, the one or more immunosuppressive agents are administered (e.g. administered daily) for the lifetime of the modified beta cell or composition in the subject. In some embodiments, the one or more immunosuppressive agents are administered (e.g. administered daily) for the lifetime of the subject. In some embodiments, the one or more immunosuppressive agents can be formulated for administration by any route known to those of skill in the art including intramuscular, intravenous, intradermal, intralesional, intraperitoneal injection, subcutaneous, intratumoral, epidural, nasal, oral, vaginal, rectal, topical, local, otic, inhalational, buccal (e.g. sublingual), and transdermal administration or any route. In some embodiments, other modes of administration also are contemplated. In some embodiments, the administration is by bolus infusion, by injection, e.g. intravenous or subcutaneous injections, intraocular injection, periocular injection, subretinal injection, intravitreal injection, trans-septal injection, subscleral injection, intrachoroidal injection, intracameral injection, subconjunctival injection, sub-Tenon's injection, retrobulbar injection, peribulbar injection, or posterior juxtascleral delivery. In some embodiments, administration is by parenteral, intrapulmonary, and intranasal, and, if desired for local treatment, intralesional administration. Parenteral infusions include intramuscular, intravenous, intraarterial, intraperitoneal, or subcutaneous administration. In some embodiments, the administration is via the portal vein. In some embodiments, the administration is by injection into the intramuscular space forearm of the subject.

In some embodiments, the one or more immunosuppressive agents may be administered at any suitable location in the subject. For example, in some embodiments, the one or more immunosuppressive agents are administered to the kidney, forearm, mouth, anus, nose, upper arm, hip, thigh, buttocks, liver, spleen, muscle, subcutaneous tissue, or white adipose tissue of the subject. In some embodiments, the one or more immunosuppressive agents are administered to the forearm of the subject. In some embodiments, the one or more immunosuppressive agents are administered to the intramuscular space of the forearm of the subject. In some embodiments, the one or more immunosuppressive agents are administered to the liver, muscle, or white adipose tissue of the subject. In some embodiments, the white adipose tissue is omentum.

### 2. Exemplary Immunosuppressive Agents

Most clinically used immunosuppressive regimens consist of a combination of one or more immunosuppressive agents used in accordance with a selected regimen. Immunosuppressive regimens can be classified as induction, maintenance, or antirejection. Induction regimens may provide intense early postoperative immune suppression (e.g. such as prior to, concurrent with, and/or after the administration of the modified beta cell or composition to the subject), while maintenance regimens are used throughout the subject's lifespan to prevent both acute and chronic rejection of the modified beta cell or composition. In some exemplary embodiments, the immunosuppressive regimens provided herein employ the highest intensity of immunosuppression (e.g. induction immunosuppression) immediately prior to, concurrent with, and/or immediately after the administration of the modified beta cell or composition to the subject with decreasing intensity over the course of about a year after administration of the modified beta cell or composition to the subject (e.g. maintenance immunosuppression), as immune reactivity and rejection probability are highest early after administration of the modified beta cell or composition and decrease over time. In such embodiments, the lowest maintenance levels of immunosuppression that are compatible with preventing rejection while minimizing drug toxicities are reached overtime. Maintenance immunosuppression may also be tapered, and in some cases fully withdrawn, in accordance with some embodiments.

Exemplary immunosuppressive agents and regimens of administration to a subject are provided herein. It should be understood that the particular immunosuppressive agents and regimens of administration described herein may be altered and optimized depending on the particular subject and/or state of the beta cell related disease or disorder. Various immunosuppressive regimens are known in the art, each of which may be applicable to the methods and used provided herein. See, for example, Markmann et al. “Phase 3 trial of human islet-after-kidney transplantation in type 1 diabetes.” Am J Transplant. 2021; 21(4): 1477-1492; Shapiro et al. “Clinical pancreatic islet transplantation.” Nature Reviews. Endocrinology. 2017; 13(5):268-277; Hering et al. “Phase 3 trial of transplantation of human islets in type 1 diabetes complicated by severe hypoglycemia.” Diabetes Care. 2016; 39(7):1230-1240; Foster et al. “Clinical Islet Transplantation Consortium. Improved health-related quality of life in a phase 3 islet transplantation trial in type 1 diabetes complicated by severe hypoglycemia.” Diabetes Care. 2018. Pii:dc171779. Doi: 10.2337/dc17-1779; Korsgren et al. “Current status of clinical islet transplantation.” Transplantation 2005; 79: 1289-1293; Shapiro et al. “Islet transplantation in seven patients with type 1 diabetes mellitus using a glucocorticoid-free immunosuppressive regimen.” N. Engl. J. Med. 2000; 343:230-238; and NIAID. “Islet transplantation in type 1 diabetes” [ClinicalTrials.gov study NCT00434811].

In some embodiments, the one or more immunosuppressive agents are a small molecule. In some embodiments, the small molecule is a chemical compound. In some embodiments, the small molecule is a nucleic acid. In some embodiments, the one or more immunosuppressive agents are a biological product. In some embodiments, the biological product is a protein. In some embodiments, the biological product is an antibody. In some embodiments, the one or more immunosuppressive agents are a pharmaceutical salt thereof and/or a preform thereof.

In some embodiments, the one or more immunosuppressive agents are one or more immunomodulatory agents. In some embodiments, the one or more immunomodulatory agents are a small molecule. In some embodiments, the small molecule is a chemical compound. In some embodiments, the small molecule is a nucleic acid. In some embodiments, the one or more immunomodulatory agents are a biological product. In some embodiments, the biological product is a protein. In some embodiments, the biological product is an antibody. In some embodiments, the one or more immunomodulatory agents are a pharmaceutical salt thereof and/or a preform thereof.

Non-limiting examples of an immunosuppressive agents include calcineurin inhibitors, steroids, alkylating agents, antibiotics, analgesics, anti-inflammatory agents, antihistamines, antiviral agents, antifungal agents, anticoagulation agents, DNA synthesis inhibitors, anti-coagulation agents, hemorheologic agents, inosine monophosphate dehydrogenase (IMDH) inhibitors, Janus kinase inhibitors, mTOR inhibitors, TNF inhibitors, and anti-CD25 inhibitors. In some embodiments, the one or more immunosuppressive agents comprise, but are not limited to, antithymocyte globulin (ATG), corticosteroids, prednisone, cortisone, prednisolone methylprednisolone, dexamethasone, betamethasone, hydrocortisone, methotrexate, acetaminophen, diphenhydramine, sirolimus (rapamycin), one or more immunosuppressive agents (FK-506), mycophenolic acid (MPA), mycophenolate mofetil (MMF), mycophenolate sodium, cyclosporine, etanercept (TNFR-Fc), azathioprine, gold salts, sulfasalazine, antimalarials, brequinar, leflunomide, mizoribine, 15-deoxyspergualine, 6-mercaptopurine, cyclophosphamide, OKT3, anti-thymocyte globulin, thymopentin (thymosin-α), fludarabine, cyclophosphamide, and an immunosuppressive antibody. Any suitable combination of any of the immunosuppressive agents, regimens, and dosages described herein may be used in the provided methods and uses, in combination with a composition comprising a modified beta cell. Antithymocyte globulin (ATG)

In some aspects, the one or more immunosuppressive agents comprise antithymocyte globulin (ATG). In some embodiments, ATG is administered to the subject (e.g. one or more regimens of ATG is administered to the subject). In some embodiments, the ATG is administered to the subject in one or more compositions, e.g. a pharmaceutical composition containing ATG. In some embodiments, the ATG is administered to the subject prior to, concurrent with, and/or after the administration of the modified beta cell or composition to the subject. In some embodiments, at least one regimen ATG is administered to the subject prior to, concurrent with, and/or after the administration of the modified beta cell or composition to the subject.

In some embodiments, the ATG is administered to the subject prior to the administration of the modified beta cell or composition to the subject. In some embodiments, the ATG is administered to the subject only prior to the administration of the modified beta cell or composition to the subject. In some embodiments, at least one regimen of ATG is administered to the subject prior to the administration of the modified beta cell or composition to the subject. In some embodiments, more than one regimen of ATG is administered to the subject prior to administration of the modified beta cell or composition to the subject. In some embodiments, the ATG (e.g. at least one regimen of the ATG) is administered between about 30 seconds and about 10 weeks prior to administration of the modified beta cell or composition to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks prior to administration of the modified beta cell or composition to the subject. In some embodiments, the ATG is administered at least about 30 seconds prior to administration of the modified beta cell or composition to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, or more, prior to administration of the modified beta cell or composition to the subject. In some embodiments, the ATG is administered less than about 10 weeks prior to administration of the modified beta cell or composition to the subject, such less than about any of 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the modified beta cell or composition to the subject. In some embodiments, the ATG is administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the modified beta cell or composition to the subject. In some embodiments, a first regimen of ATG is administered to the subject about 2 days prior to administration of the modified beta cell or composition to the subject. In some embodiments, a first regimen of ATG is administered to the subject about 1 day prior to administration of the modified beta cell or composition to the subject. In some embodiments, a first regimen of ATG is administered to the subject about 2 days prior to administration of the modified beta cell or composition to the subject, and a second regimen of ATG is administered to the subject about 1 day prior to administration of the modified beta cell or composition to the subject.

In some embodiments, the ATG is administered to the subject prior to administration of the modified beta cell or composition, and continued to be administered over the course of the subject's lifespan. In some embodiments, the ATG is administered to the subject prior to each round of administration of the modified beta cell or composition. In some embodiments, the ATG is administered to the subject prior to each round of administration of the modified beta cell or composition, and continued to be administered over the course of the subject's lifespan.

In some embodiments, the ATG (e.g. a regimen of ATG) is administered to the subject on the same day as the administration of the modified beta cell or composition to the subject. In some embodiments, at least one regimen of ATG is administered to the subject on the same day as the administration of the modified beta cell or composition to the subject. In some embodiments, the ATG is administered to the subject concurrent with administration of the modified beta cell or composition to the subject. In some embodiments, at least one regimen of ATG is administered to the subject concurrent with administration of the modified beta cell or composition to the subject. In some embodiments, the ATG is administered to the subject on the same day as administration of the modified beta cell or composition to the subject, and continued to be administered over the course of the subject's lifespan. In some embodiments, the ATG is administered to the subject concurrent with administration of the modified beta cell or composition to the subject, and continued to be administered over the course of the subject's lifespan. In some embodiments, a first regimen of the ATG is administered to the subject concurrent with administration of the modified beta cell or composition to the subject. In some embodiments, a second regimen the ATG is administered to the subject concurrent with administration of the modified beta cell or composition to the subject. In some embodiments, the ATG is administered to the subject on the same day as each round of administration of the modified beta cell or composition. In some embodiments, the ATG is administered to the subject on the same day as each round of administration of the modified beta cell or composition, and continued to be administered over the course of the subject's lifespan. In some embodiments, the ATG is administered to the subject concurrent with each round of administration of the modified beta cell or composition. In some embodiments, the ATG is administered to the subject concurrent with each round of administration of the modified beta cell or composition, and continued to be administered over the course of the subject's lifespan.

In some embodiments, the ATG (e.g. a regimen of ATG) is administered to the subject after administration of the modified beta cell or composition to the subject. In some embodiments, at least one regimen of ATG is administered to the subject after administration of the modified beta cell or composition to the subject. In some embodiments, more than one regimen of ATG is administered to the subject after administration of the modified beta cell or composition to the subject. In some embodiments, the ATG is administered to the subject only after administration of a first and/or second regimen of the modified beta cell or composition to the subject. In some embodiments, the ATG is administered between about 30 seconds and about 10 weeks after administration of the modified beta cell or composition to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks after administration of the modified beta cell or composition to the subject. In some embodiments, the ATG is administered at least about 30 seconds after administration of the modified beta cell or composition to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, or more, after administration of the modified beta cell or composition to the subject. In some embodiments, the ATG is administered less than about 10 weeks after administration of the modified beta cell or composition to the subject, such less than about any of 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the modified beta cell or composition to the subject. In some embodiments, the ATG is administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds after administration of the modified beta cell or composition to the subject. In some embodiments, the ATG is administered to the subject 48 hours after administration of the modified beta cell or composition. In some embodiments, the ATG is administered to the subject after administration of the modified beta cell or composition, and continued to be administered over the course of the subject's lifespan. In some embodiments, the ATG is administered to the subject after each round of administration of the modified beta cell or composition. In some embodiments, the ATG is administered to the subject after each round of administration of the modified beta cell or composition, and continued to be administered over the course of the subject's lifespan.

In some embodiments, the ATG is administered to the subject prior to and after the administration of the modified beta cell or composition to the subject. In some embodiments, the ATG is administered to the subject prior to, on the same day, and after the administration of the modified beta cell or composition to the subject. In some embodiments, the ATG is administered to the subject prior to, concurrent with, and after the administration of the modified beta cell or composition to the subject. In some embodiments, the ATG is administered to the subject: i) about 2 days prior; ii) about 1 day prior; iii) on the same day; iv) about 1 day after; and/or, v) about 2 days after the administration of the composition comprising the modified beta cell to the subject.

In some embodiments, a regimen and/or the total daily dose of between about 0.05 mg/kg and about 4.0 mg/kg ATG is administered to the subject, such as a regimen of between about 0.05 mg/kg and about 1.0 mg/kg ATG, between about 0.1 mg/kg and about 2.0 mg/kg, between about 1.0 mg/kg and about 3.0 mg/kg, or between about 2.0 mg/kg and about 4.0 mg/kg. In some embodiments, a regimen of between about 0.1 mg/kg and about 2.0 mg/kg ATG is administered to the subject. In some embodiments, a regimen of greater than about 0.05 mg/kg of ATG is administered to the subject, such as a regimen of greater than any of about 0.1 mg/kg, 0.2 mg/kg, 0.3 mg/kg, 0.4 mg/kg, 0.5 mg/kg, 1.0 mg/kg, 1.5 mg/kg, 2.0 mg/kg, 2.5 mg/kg, 3.0 mg/kg, 3.5 mg/kg, 4.0 mg/kg, or greater, of ATG. In some embodiments, a regimen of less than about 4.0 mg/kg of ATG is administered to the subject, such as a regimen of less than any of about 3.5 mg/kg, 3.0 mg/kg, 2.5 mg/kg, 2.0 mg/kg, 1.5 mg/kg, 1.0 mg/kg, 0.5 mg/kg, 0.4 mg/kg, 0.3 mg/kg, 0.2 mg/kg, 0.1 mg/kg, 0.05 mg/kg, or less, of ATG. In some embodiments, a regimen of about 0.5 mg/kg ATG is administered to the subject. In some embodiments, a regimen of about 1.0 mg/kg ATG is administered to the subject. In some embodiments, a regimen of about 1.5 mg/kg ATG is administered to the subject.

In some embodiments, a regimen of about 0.5 mg/kg of ATG is administered to the subject about 2 days prior to the administration of the modified beta cell or composition to the subject. In some embodiments, a regimen of about 1.0 mg/kg of ATG is administered to the subject about 1 day prior to the administration of the modified beta cell or composition to the subject. In some embodiments, a regimen of about 1.5 mg/kg of ATG is administered to the subject on the same day as the administration of the modified beta cell or composition to the subject. In some embodiments, a regimen of about 1.5 mg/kg of ATG is administered to the subject 1 day after the administration of the modified beta cell or composition to the subject. In some embodiments, a regimen of about 1.5 mg/kg of ATG is administered to the subject about 2 days after the administration of the modified beta cell or composition to the subject. In some embodiments, i) a regimen of about 0.5 mg/kg of ATG is administered to the subject about 2 days prior to the administration of the modified beta cell or composition to the subject; ii) a regimen of about 1.0 mg/kg of ATG is administered to the subject about 1 day prior to the administration of the modified beta cell or composition to the subject; and, iii) a regimen of about 1.5 mg/kg of ATG is administered to the subject on the same day as the administration of the modified beta cell or composition to the subject, about 1 day after the administration of the modified beta cell or composition to the subject, and about 2 days after the administration of the modified beta cell or composition to the subject. In some embodiments, the ATG is administered to the subject at a lower dose.

a. Steroids

In some aspects, the one or more immunosuppressive agents comprise a steroid (e.g. one or more steroids). Steroids may be used to reduce inflammation in a subject. In some embodiments, the steroid is a corticosteroid. In some aspects, the one or more immunosuppressive agents comprise prednisone, cortisone, prednisolone methylprednisolone, dexamethasone, betamethasone, and/or hydrocortisone. In some embodiments, the steroid is administered to the subject (e.g. one or more regimens of one or more steroids is administered to the subject). In some embodiments, one or more steroids are administered to the subject in one or more compositions, e.g. a pharmaceutical composition containing one or more steroids. In some embodiments, the one or more steroids are administered to the subject prior to, concurrent with, and/or after the administration of the modified beta cell or composition (e.g., composition comprising modified beta cells) to the subject. In some embodiments, at least one regimen of the steroid is administered to the subject prior to, concurrent with, and/or after the administration of the modified beta cell or composition to the subject.

In some embodiments, the one or more immunosuppressive agents do not comprise a steroid. In some embodiments, a subject previously on or currently on steroidal treatment is not suitable for treatment with the any of the methods or uses provided herein.

In some embodiments, the one or more immunosuppressive agents comprise methylprednisolone. In some embodiments, the methylprednisolone (e.g. a regimen of methylprednisolone) is administered to the subject prior to, concurrent with, and/or after the administration of the modified beta cell or composition to the subject. In some embodiments, at least one regimen of methylprednisolone is administered to the subject prior to, concurrent with, and/or after the administration of the modified beta cell or composition to the subject.

In some embodiments, the methylprednisolone (e.g. a regimen of methylprednisolone) is administered to the subject prior to the administration of the modified beta cell or composition to the subject. In some embodiments, the methylprednisolone is administered to the subject only prior to the administration of the modified beta cell or composition to the subject. In some embodiments, at least one regimen of methylprednisolone is administered to the subject prior to the administration of the modified beta cell or composition to the subject. In some embodiments, more than one regimen of methylprednisolone is administered to the subject prior to administration of the modified beta cell or composition to the subject. In some embodiments, the methylprednisolone (e.g. at least one regimen of the methylprednisolone) is administered between about 30 seconds and about 10 weeks prior to administration of the modified beta cell or composition to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks prior to administration of the modified beta cell or composition to the subject. In some embodiments, the methylprednisolone is administered at least about 30 seconds prior to administration of the modified beta cell or composition to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, or more, prior to administration of the modified beta cell or composition to the subject. In some embodiments, the methylprednisolone is administered less than about 10 weeks prior to administration of the modified beta cell or composition to the subject, such less than about any of 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the modified beta cell or composition to the subject. In some embodiments, the methylprednisolone is administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the modified beta cell or composition to the subject. In some embodiments, a regimen of methylprednisolone is administered to the subject about 2 days prior to administration of the modified beta cell or composition to the subject.

In some embodiments, a regimen of methylprednisolone is administered to the subject prior to administration of a regimen of ATG to the subject. In some embodiments, the regimen of methylprednisolone is only administered to the subject prior to administration of a regimen of ATG to the subject. In some embodiments, the regimen of methylprednisolone is administered to the subject prior to administration of a first regimen ATG to the subject. In some embodiments, the regimen of methylprednisolone is only administered to the subject prior to administration of a first regimen ATG to the subject. In some embodiments, the regimen of methylprednisolone is administered to the subject between about 30 minutes and about 24 hours prior to the administration of a regimen (e.g. a first regimen) of ATG to the subject, such as between about 30 minutes and about 5 hours, between about 1 hour and about 3 hours, between about 4 hours and about 10 hours, or between about 8 hours and about 24 hours prior to the administration of a regimen of ATG to the subject. In some embodiments, the regimen of methylprednisolone is administered to the subject about 1 hour prior to the administration of a regimen of ATG to the subject. In some embodiments, the regimen of methylprednisolone is administered to the subject about 1 hour prior to the administration of a first regimen of ATG to the subject. In some embodiments, the regimen of methylprednisolone is administered to the subject concurrent with the administration of a regimen of ATG to the subject. In some embodiments, the regimen of methylprednisolone is administered to the subject concurrent with the administration of a first regimen of ATG to the subject. In some embodiments, the regimen of methylprednisolone is administered to the subject about midway through the administration of a regimen (e.g. a first regimen) of ATG to the subject. In some embodiments, the regimen of methylprednisolone is administered to the subject prior to administration of regimen of methylprednisolone is administered to the subject prior to administration of ATG to the subject, and prior to administration of the modified beta cell or composition to the subject. In some embodiments, the regimen of methylprednisolone and the regimen ATG are both administered to subject prior to administration of the modified beta cell or composition to the subject.

In some embodiments, a regimen and/or the total daily dose of between about 0.05 mg/kg and about 4.0 mg/kg methylprednisolone is administered to the subject, such as a regimen of between about 0.05 mg/kg and about 1.0 mg/kg methylprednisolone, between about 0.1 mg/kg and about 2.0 mg/kg, between about 1.0 mg/kg and about 3.0 mg/kg, or between about 2.0 mg/kg and about 4.0 mg/kg. In some embodiments, a regimen of between about 0.1 mg/kg and about 2.0 mg/kg of methylprednisolone is administered to the subject. In some embodiments, a regimen of greater than about 0.05 mg/kg of methylprednisolone is administered to the subject, such as a regimen of greater than any of about 0.1 mg/kg, 0.2 mg/kg, 0.3 mg/kg, 0.4 mg/kg, 0.5 mg/kg, 1.0 mg/kg, 1.5 mg/kg, 2.0 mg/kg, 2.5 mg/kg, 3.0 mg/kg, 3.5 mg/kg, 4.0 mg/kg, or greater, of methylprednisolone. In some embodiments, a regimen of less than about 4.0 mg/kg of methylprednisolone is administered to the subject, such as a regimen of less than any of about 3.5 mg/kg, 3.0 mg/kg, 2.5 mg/kg, 2.0 mg/kg, 1.5 mg/kg, 1.0 mg/kg, 0.5 mg/kg, 0.4 mg/kg, 0.3 mg/kg, 0.2 mg/kg, 0.1 mg/kg, 0.05 mg/kg, or less, of methylprednisolone. In some embodiments, a regimen of about 1.0 mg/kg methylprednisolone is administered to the subject. In some embodiments, the methylprednisolone is administered to the subject intravenously.

In some embodiments, a regimen of about 1.0 mg/kg of methylprednisolone is administered to the subject about 1 hour prior to the administration of a first regimen ATG to the subject. In some embodiments, a regimen of about 1.0 mg/kg of methylprednisolone is administered to the subject about midway through the administration of the first regimen ATG to the subject. In some embodiments, the first regimen of about 0.5 mg/kg of ATG is administered to the subject about 2 days prior to the administration of the modified beta cell or composition to the subject. In some embodiments, a regimen of about 1.0 mg/kg of ATG is administered to the subject about 1 day prior to the administration of the modified beta cell or composition to the subject. In some embodiments, a regimen of about 1.5 mg/kg of ATG is administered to the subject on the same day as the administration of the modified beta cell or composition to the subject. In some embodiments, a regimen of about 1.5 mg/kg of ATG is administered to the subject 1 day after the administration of the modified beta cell or composition to the subject. In some embodiments, a regimen of about 1.5 mg/kg of ATG is administered to the subject about 2 days after the administration of the modified beta cell or composition to the subject. In some embodiments, i) a regimen of about 1.0 mg/kg of methylprednisolone is administered to the subject about 1 hour prior to the administration of a first regimen ATG to the subject; ii) a regimen of about 1.0 mg/kg of methylprednisolone is administered to the subject about midway through the administration of the first regimen ATG to the subject; iii) a regimen of about 0.5 mg/kg of ATG is administered to the subject about 2 days prior to the administration of the modified beta cell or composition to the subject; iv) a regimen of about 1.0 mg/kg of ATG is administered to the subject about 1 day prior to the administration of the modified beta cell or composition to the subject; and/or, v) a regimen of about 1.5 mg/kg of ATG is administered to the subject on the same day as the administration of the modified beta cell or composition to the subject, about 1 day after the administration of composition comprising a the modified beta cell to the subject, and about 2 days after the administration of the modified beta cell or composition to the subject. In some embodiments, the regimen of methylprednisolone is administered at a lower dose. In some embodiments, the regimen of ATG is administered at a lower dose.

b. Analgesics

In some aspects, the one or more immunosuppressive agents comprise an analgesic (e.g., one or more analgesics). Analgesics are drugs that may be used to relieve pain. In some embodiments, the analgesic is acetaminophen, an opioid, or a non-steroidal anti-inflammatory drug (NSAID). In some embodiments, the analgesic is administered to the subject (e.g. one or more regimens of the analgesic is administered to the subject). In some embodiments, the analgesic is administered to the subject in one or more compositions, e.g. a pharmaceutical composition containing the analgesic. In some embodiments, the analgesic is administered to the subject prior to, concurrent with, and/or after the administration of the modified beta cell or composition (e.g., composition comprising modified beta cells) to the subject. In some embodiments, at least one regimen of the analgesic is administered to the subject prior to, concurrent with, and/or after the administration of the modified beta cell or composition to the subject.

In some embodiments, the one or more immunosuppressive agents comprise acetaminophen. In some embodiments, the acetaminophen is administered to the subject prior to, concurrent with, and/or after the administration of the modified beta cell or composition to the subject. In some embodiments, at least one regimen of acetaminophen is administered to the subject prior to, concurrent with, and/or after the administration of the modified beta cell or composition to the subject.

In some embodiments, the acetaminophen is administered to the subject prior to the administration of the modified beta cell or composition to the subject. In some embodiments, the acetaminophen is administered to the subject only prior to the administration of the modified beta cell or composition to the subject. In some embodiments, at least one regimen of acetaminophen is administered to the subject prior to the administration of the modified beta cell or composition to the subject. In some embodiments, more than one regimen of acetaminophen is administered to the subject prior to administration of the modified beta cell or composition to the subject. In some embodiments, the acetaminophen (e.g. at least one regimen of the acetaminophen) is administered between about 30 seconds and about 10 weeks prior to administration of the modified beta cell or composition to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks prior to administration of the modified beta cell or composition to the subject. In some embodiments, the acetaminophen is administered at least about 30 seconds prior to administration of the modified beta cell or composition to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, or more, prior to administration of the modified beta cell or composition to the subject. In some embodiments, the acetaminophen is administered less than about 10 weeks prior to administration of the modified beta cell or composition to the subject, such less than about any of 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the modified beta cell or composition to the subject. In some embodiments, the acetaminophen is administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the modified beta cell or composition to the subject. In some embodiments, a regimen of acetaminophen is administered to the subject about 2 days prior to administration of the modified beta cell or composition to the subject.

In some embodiments, a regimen of acetaminophen is administered to the subject prior to administration of a regimen of ATG to the subject. In some embodiments, the regimen of acetaminophen is only administered to the subject prior to administration of a regimen of ATG to the subject. In some embodiments, the regimen of acetaminophen is administered to the subject prior to administration of a first regimen ATG to the subject. In some embodiments, the regimen of acetaminophen is only administered to the subject prior to administration of a first regimen ATG to the subject. In some embodiments, the regimen of acetaminophen is administered to the subject between about 30 minutes and about 24 hours prior to the administration of a regimen (e.g. a first regimen) of ATG to the subject, such as between about 30 minutes and about 5 hours, between about 1 hour and about 3 hours, between about 4 hours and about 10 hours, or between about 8 hours and about 24 hours prior to the administration of a regimen of ATG to the subject. In some embodiments, the regimen of acetaminophen is administered to the subject about 30 minutes prior to the administration of a regimen of ATG to the subject. In some embodiments, the regimen of acetaminophen is administered to the subject about 30 minutes prior to the administration of a first regimen of ATG to the subject. In some embodiments, the regimen of acetaminophen is administered to the subject concurrent with the administration of a regimen of ATG to the subject. In some embodiments, the regimen of acetaminophen is administered to the subject concurrent with the administration of a first regimen of ATG to the subject. In some embodiments, the regimen of acetaminophen is administered to the subject about midway through the administration of a regimen (e.g. a first regimen) of ATG to the subject. In some embodiments, the regimen of acetaminophen is administered to the subject prior to administration of regimen of acetaminophen is administered to the subject prior to administration of ATG to the subject, and prior to administration of the modified beta cell or composition to the subject. In some embodiments, the regimen of acetaminophen and the regimen ATG are both administered to subject prior to administration of the modified beta cell or composition to the subject.

In some embodiments, a regimen and/or the total daily dose of between about 10 mg and about 5,000 mg acetaminophen is administered to the subject, such as a regimen of between about 10 mg and about 100 mg acetaminophen, between about 100 mg and about 1,000 mg, or between about 500 mg and about 5,000 mg. In some embodiments, a regimen of between about 100 and about 10,000 mg of acetaminophen is administered to the subject. In some embodiments, a regimen of greater than about 10 mg of acetaminophen is administered to the subject, such as a regimen of greater than any of about 20 mg, 30 mg, 40 mg, 50 mg, 100 mg, 500 mg, 1,000 mg, 2,000 mg, 3,000 mg, 4,000 mg, 5,000 mg, or greater, of acetaminophen. In some embodiments, a regimen of less than about 5,000 mg of acetaminophen is administered to the subject, such as a regimen of less than any of about 4,000 mg, 3,000 mg, 2,000 mg, 1,000 mg, 500 mg, 100 mg, 50 mg, 40 mg, 30 mg, 20 mg, 10 mg, or less, of acetaminophen. In some embodiments, a regimen of about 650 mg acetaminophen is administered to the subject. In some embodiments, acetaminophen is administered to the subject orally or rectally.

In some embodiments, a regimen of about 650 mg of acetaminophen is administered to the subject about 30 minutes prior to the administration of a first regimen ATG to the subject. In some embodiments, a regimen of about 650 mg of acetaminophen is administered to the subject about midway through the administration of the first regimen ATG to the subject. In some embodiments, a regimen of about 0.5 mg/kg of ATG is administered to the subject about 2 days prior to the administration of the modified beta cell or composition to the subject. In some embodiments, a regimen of about 1.0 mg/kg of ATG is administered to the subject about 1 day prior to the administration of the modified beta cell or composition to the subject. In some embodiments, a regimen of about 1.5 mg/kg of ATG is administered to the subject on the same day as the administration of the modified beta cell or composition to the subject. In some embodiments, a regimen of about 1.0 mg/kg of ATG is administered to the subject about 1 day after the administration of the modified beta cell or composition to the subject. In some embodiments, a regimen of about 1.0 mg/kg of ATG is administered to the subject about 2 days after the administration of the modified beta cell or composition to the subject. In some embodiments, i) a regimen of about 650 mg of acetaminophen is administered to the subject about 30 minutes prior to the administration of a first regimen ATG to the subject; ii) a regimen of about 650 mg of acetaminophen is administered to the subject about midway through the administration of the first regimen ATG to the subject; iii) a regimen of about 0.5 mg/kg of ATG is administered to the subject about 2 days prior to the administration of the modified beta cell or composition to the subject; iv) a regimen of about 1.0 mg/kg of ATG is administered to the subject about 1 day prior to the administration of the modified beta cell or composition to the subject; and/or, v) a regimen of about 1.5 mg/kg of ATG is administered to the subject on the same day as the administration of the modified beta cell or composition to the subject, about 1 day after the administration of the modified beta cell or composition to the subject, and about 2 days after the administration of the modified beta cell or composition to the subject. In some embodiments, the acetaminophen is administered at a lower dose. In some embodiments, the ATG is administered at a lower dose.

c. Antihistamines

In some aspects, the one or more immunosuppressive agents comprise an antihistamine (e.g., one or more antihistamines). Antihistamines are drugs that may be used to relieve allergy symptoms such as runny nose, sneezing, and congestion. In some embodiments, the antihistamine is a Hi-antihistamine, a H2-antihistamine, a H3-antihistamine, a H4-antihistamine, a histidine decarboxylase inhibitor, or a mast cell inhibitor. In some embodiments, the antihistamine may be, but is not limited to, diphenhydramine, doxylamine, hydroxyzine, promethazine, phenyltoloxamine, orphenadrine, tripelennamine, cimetidine, clobenpropit, thioperamide, cromolyn sodium, or catechin. In some embodiments, the antihistamine is administered to the subject (e.g. one or more regimens of the antihistamine is administered to the subject). In some embodiments, the antihistamine is administered to the subject in one or more compositions, e.g. a pharmaceutical composition containing the antihistamine. In some embodiments, the antihistamine is administered to the subject prior to, concurrent with, and/or after the administration of the modified beta cell or composition to the subject. In some embodiments, at least one regimen of the antihistamine is administered to the subject prior to, concurrent with, and/or after the administration of the modified beta cell or composition to the subject.

In some embodiments, the one or more immunosuppressive agents comprise diphenhydramine. In some embodiments, the diphenhydramine (e.g. a regimen of diphenhydramine) is administered to the subject prior to, concurrent with, and/or after the administration of the modified beta cell or composition to the subject. In some embodiments, at least one regimen of diphenhydramine is administered to the subject prior to, concurrent with, and/or after the administration of the modified beta cell or composition to the subject.

In some embodiments, the diphenhydramine (e.g. a regimen of diphenhydramine) is administered to the subject prior to the administration of the modified beta cell or composition to the subject. In some embodiments, the diphenhydramine is administered to the subject only prior to the administration of the modified beta cell or composition to the subject. In some embodiments, at least one regimen of diphenhydramine is administered to the subject prior to the administration of the modified beta cell or composition to the subject. In some embodiments, more than one regimen of diphenhydramine is administered to the subject prior to administration of the modified beta cell or composition to the subject. In some embodiments, the diphenhydramine (e.g. at least one regimen of the diphenhydramine) is administered between about 30 seconds and about 10 weeks prior to administration of the modified beta cell or composition to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks prior to administration of the modified beta cell or composition to the subject. In some embodiments, the diphenhydramine is administered at least about 30 seconds prior to administration of the modified beta cell or composition to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, or more, prior to administration of the modified beta cell or composition to the subject. In some embodiments, the diphenhydramine is administered less than about 10 weeks prior to administration of the modified beta cell or composition to the subject, such less than about any of 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the modified beta cell or composition to the subject. In some embodiments, the diphenhydramine is administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the modified beta cell or composition to the subject. In some embodiments, a regimen of diphenhydramine is administered to the subject about 2 days prior to administration of the modified beta cell or composition to the subject.

In some embodiments, a regimen of diphenhydramine is administered to the subject prior to administration of a regimen of ATG to the subject. In some embodiments, the regimen of diphenhydramine is only administered to the subject prior to administration of a regimen of ATG to the subject. In some embodiments, the regimen of diphenhydramine is administered to the subject prior to administration of a first regimen ATG to the subject. In some embodiments, the regimen of diphenhydramine is only administered to the subject prior to administration of a first regimen ATG to the subject. In some embodiments, the regimen of diphenhydramine is administered to the subject between about 30 minutes and about 24 hours prior to the administration of a regimen (e.g. a first regimen) of ATG to the subject, such as between about 30 minutes and about 5 hours, between about 1 hour and about 3 hours, between about 4 hours and about 10 hours, or between about 8 hours and about 24 hours prior to the administration of a regimen of ATG to the subject. In some embodiments, the regimen of diphenhydramine is administered to the subject about 30 minutes prior to the administration of a regimen of ATG to the subject. In some embodiments, the regimen of diphenhydramine is administered to the subject about 30 minutes prior to the administration of a first regimen of ATG to the subject. In some embodiments, the regimen of diphenhydramine is administered to the subject concurrent with the administration of a regimen of ATG to the subject. In some embodiments, the regimen of diphenhydramine is administered to the subject concurrent with the administration of a first regimen of ATG to the subject. In some embodiments, the regimen of diphenhydramine is administered to the subject about midway through the administration of a regimen (e.g. a first regimen) of ATG to the subject. In some embodiments, the regimen of diphenhydramine is administered to the subject prior to administration of regimen of diphenhydramine is administered to the subject prior to administration of ATG to the subject, and prior to administration of the modified beta cell or composition to the subject. In some embodiments, the regimen of diphenhydramine and the regimen ATG are both administered to subject prior to administration of the modified beta cell or composition to the subject.

In some embodiments, a regimen and/or a total daily dose of between about 1 mg and about 1,000 mg diphenhydramine is administered to the subject, such as a regimen of between about 1 mg and about 100 mg diphenhydramine, between about 50 mg and about 500 mg, or between about 500 mg and about 1,000 mg. In some embodiments, a regimen of between about 10 and about 100 mg of diphenhydramine is administered to the subject. In some embodiments, a regimen of greater than about 1 mg of diphenhydramine is administered to the subject, such as a regimen of greater than any of about 2 mg, 3 mg, 4 mg, 5 mg, 10 mg, 50 mg, 100 mg, 200 mg, 300 mg, 400 mg, 500 mg, 1,000 mg or greater, of diphenhydramine. In some embodiments, a regimen of less than about 1,000 mg of diphenhydramine is administered to the subject, such as a regimen of less than any of about 500 mg, 400 mg, 300 mg, 200 mg, 100 mg, 50 mg, 10 mg, 5 mg, 4 mg, 3 mg, 2 mg, 1 mg, or less, of diphenhydramine. In some embodiments, a regimen of about 50 mg diphenhydramine is administered to the subject. In some embodiments, diphenhydramine is administered to the subject orally or rectally.

In some embodiments, a regimen of about 50 mg of a diphenhydramine is administered to the subject about 30 minutes prior to the administration of a first regimen ATG to the subject. In some embodiments, a regimen of about 50 mg of diphenhydramine is administered to the subject about midway through the administration of the first regimen ATG to the subject. In some embodiments, a regimen of about 0.5 mg/kg of ATG is administered to the subject about 2 days prior to the administration of the modified beta cell or composition to the subject. In some embodiments, a regimen of about 1.0 mg/kg of ATG is administered to the subject about 1 day prior to the administration of the modified beta cell or composition to the subject. In some embodiments, a regimen of about 1.5 mg/kg of ATG is administered to the subject on the same day as the administration of the modified beta cell or composition to the subject. In some embodiments, a regimen of about 1.5 mg/kg of ATG is administered to the subject about 1 day after the administration of the modified beta cell or composition to the subject. In some embodiments, a regimen of about 1.5 mg/kg of ATG is administered to the subject 2 days after the administration of the modified beta cell or composition to the subject. In some embodiments, i) a regimen of about 50 mg of a diphenhydramine is administered to the subject about 30 minutes prior to the administration of a first regimen ATG to the subject; ii) a regimen of about 50 mg of diphenhydramine is administered to the subject about midway through the administration of the first regimen ATG to the subject; iii) a regimen of about 0.5 mg/kg of ATG is administered to the subject about 2 days prior to the administration of the modified beta cell or composition to the subject; iv) a regimen of about 1.0 mg/kg of ATG is administered to the subject about 1 day prior to the administration of the modified beta cell or composition to the subject; and/or, v) a regimen of about 1.5 mg/kg of ATG is administered to the subject on the same day as the administration of the modified beta cell or composition to the subject, about 1 day after the administration of the modified beta cell or composition to the subject, and about 2 days after the administration of the modified beta cell or composition to the subject. In some embodiments, the diphenhydramine is administered at a lower dose. In some embodiments, the ATG is administered at a lower dose.

d. Anti-inflammatory Agents

In some aspects, the one or more immunosuppressive agents comprise an anti-inflammatory agent (e.g., one or more anti-inflammatory agents). Anti-inflammatory agents are drugs that may be used to reduce inflammation (redness, swelling, and pain) in a subject. In some embodiments, the anti-inflammatory agent is dexamethasone. In some embodiments, the anti-inflammatory agent is a tumor necrosis factor (TNF) inhibitor. The TNF inhibitor may be, but is not limited to, infliximab, adalimumab, etanercept (TNFR-Fc), golimumab, and certolizumab. In some embodiments, the TNF inhibitor is etanercept. In some embodiments, the anti-inflammatory agent is administered to the subject (e.g. one or more regimens of the anti-inflammatory agent is administered to the subject). In some embodiments, the anti-inflammatory agent is administered to the subject in one or more compositions, e.g. a pharmaceutical composition containing the anti-inflammatory agent. In some embodiments, the anti-inflammatory agent is administered to the subject prior to, concurrent with, and/or after the administration of the modified beta cell or composition (e.g., composition comprising modified beta cells) to the subject. In some embodiments, at least one regimen of the anti-inflammatory agent is administered to the subject prior to, concurrent with, and/or after the administration of the modified beta cell or composition to the subject.

In some embodiments, the one or more immunosuppressive agents comprise dexamethasone. In some embodiments, the dexamethasone (e.g. a regimen of dexamethasone) is administered to the subject prior to, concurrent with, and/or after the administration of the modified beta cell or composition to the subject. In some embodiments, at least one regimen of dexamethasone is administered to the subject prior to, concurrent with, and/or after the administration of the modified beta cell or composition to the subject.

In some embodiments, the one or more immunosuppressive agents comprise etanercept. In some embodiments, the etanercept (e.g. a regimen of etanercept) is administered to the subject prior to, concurrent with, and/or after the administration of the modified beta cell or composition to the subject. In some embodiments, at least one regimen of etanercept is administered to the subject prior to, concurrent with, and/or after the administration of the modified beta cell or composition to the subject.

In some embodiments, the etanercept (e.g. a regimen of etanercept) is administered to the subject prior to the administration of the modified beta cell or composition to the subject. In some embodiments, the etanercept is administered to the subject only prior to the administration of the modified beta cell or composition to the subject. In some embodiments, at least one regimen of etanercept is administered to the subject prior to the administration of the modified beta cell or composition to the subject. In some embodiments, more than one regimen of etanercept is administered to the subject prior to administration of the modified beta cell or composition to the subject. In some embodiments, the etanercept (e.g. at least one regimen of the etanercept) is administered between about 30 seconds and about 10 weeks prior to administration of the modified beta cell or composition to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks prior to administration of the modified beta cell or composition to the subject. In some embodiments, the etanercept is administered at least about 30 seconds prior to administration of the modified beta cell or composition to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, or more, prior to administration of the modified beta cell or composition to the subject. In some embodiments, the etanercept is administered less than about 10 weeks prior to administration of the modified beta cell or composition to the subject, such less than about any of 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the modified beta cell or composition to the subject. In some embodiments, the etanercept is administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the modified beta cell or composition to the subject.

In some embodiments, the etanercept (e.g. a regimen of etanercept, such as a first regimen) is administered to the subject on the same day as the administration of the modified beta cell or composition to the subject. In some embodiments, at least one regimen of etanercept is administered to the subject on the same day as the administration of the modified beta cell or composition to the subject. In some embodiments, the etanercept is administered to the subject concurrent with administration of the modified beta cell or composition to the subject. In some embodiments, at least one regimen of etanercept is administered to the subject concurrent with administration of the modified beta cell or composition to the subject. In some embodiments, the etanercept is administered to the subject on the same day as administration of the modified beta cell or composition to the subject, and continued to be administered over the course of the subject's lifespan. In some embodiments, the etanercept is administered to the subject concurrent with administration of the modified beta cell or composition to the subject, and continued to be administered over the course of the subject's lifespan. In some embodiments, a first regimen of the etanercept is administered to the subject concurrent with administration of the modified beta cell or composition to the subject. In some embodiments, a second regimen the etanercept is administered to the subject concurrent with administration of the modified beta cell or composition to the subject. In some embodiments, the etanercept is administered to the subject on the same day as each round of administration of the modified beta cell or composition. In some embodiments, the etanercept is administered to the subject on the same day as each round of administration of the modified beta cell or composition, and continued to be administered over the course of the subject's lifespan. In some embodiments, the etanercept is administered to the subject concurrent with each round of administration of the modified beta cell or composition. In some embodiments, the etanercept is administered to the subject concurrent with each round of administration of the modified beta cell or composition, and continued to be administered over the course of the subject's lifespan.

In some embodiments, the etanercept (e.g. a regimen of etanercept) is administered to the subject after administration of the modified beta cell or composition to the subject. In some embodiments, at least one regimen of etanercept is administered to the subject after administration of the modified beta cell or composition to the subject. In some embodiments, more than one regimen of etanercept is administered to the subject after administration of the modified beta cell or composition to the subject. In some embodiments, the etanercept is administered to the subject only after administration of a first and/or second regimen of the modified beta cell or composition to the subject. In some embodiments, the etanercept is administered between about 30 seconds and about 10 weeks after administration of the modified beta cell or composition to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks after administration of the modified beta cell or composition to the subject. In some embodiments, the etanercept is administered at least about 30 seconds after administration of the modified beta cell or composition to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, or more, after administration of the modified beta cell or composition to the subject. In some embodiments, the etanercept is administered less than about 10 weeks after administration of the modified beta cell or composition to the subject, such less than about any of 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the modified beta cell or composition to the subject. In some embodiments, the etanercept is administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds after administration of the modified beta cell or composition to the subject. In some embodiments, the etanercept is administered to the subject about 3 days after administration of the modified beta cell or composition. In some embodiments, the etanercept is administered to the subject about 7 days after administration of the modified beta cell or composition. In some embodiments, the etanercept is administered to the subject about 10 days after administration of the modified beta cell or composition. In some embodiments, the etanercept is administered to the subject about 3 days, about 7 days, and about 10 days after administration of the modified beta cell or composition. In some embodiments, the etanercept is administered to the subject after administration of the modified beta cell or composition, and continued to be administered over the course of the subject's lifespan. In some embodiments, the etanercept is administered to the subject after each round of administration of the modified beta cell or composition. In some embodiments, the etanercept is administered to the subject after each round of administration of the modified beta cell or composition, and continued to be administered over the course of the subject's lifespan.

In some embodiments, the etanercept is administered to the subject on the same day and after the administration of the modified beta cell or composition to the subject. In some embodiments, the etanercept is administered to the subject concurrent with and after the administration of the modified beta cell or composition to the subject. In some embodiments, the etanercept is administered to the subject: i) on the same day; ii) about 3 days after; iii) about 7 days after; and/or iv) about 10 days after the administration of the composition comprising modified beta cell to the subject.

In some embodiments, a regimen and/or the total daily dose of between about 1 mg and about 1,000 mg etanercept is administered to the subject, such as a regimen of between about 1 mg and about 100 mg etanercept, between about 50 mg and about 500 mg, or between about 500 mg and about 1,000 mg. In some embodiments, a regimen of between about 10 and about 100 mg of etanercept is administered to the subject. In some embodiments, a regimen of greater than about 1 mg of etanercept is administered to the subject, such as a regimen of greater than any of about 2 mg, 3 mg, 4 mg, 5 mg, 10 mg, 50 mg, 100 mg, 200 mg, 300 mg, 400 mg, 500 mg, 1,000 mg or greater, of etanercept. In some embodiments, a regimen of less than about 1,000 mg of etanercept is administered to the subject, such as a regimen of less than any of about 500 mg, 400 mg, 300 mg, 200 mg, 100 mg, 50 mg, 10 mg, 5 mg, 4 mg, 3 mg, 2 mg, 1 mg, or less, of etanercept. In some embodiments, a regimen of about 50 mg etanercept is administered to the subject. In some embodiments, a regimen of about 25 mg etanercept is administered to the subject.

In some embodiments, a regimen of about 50 mg of etanercept is administered to the subject on the same day as the administration of the modified beta cell or composition to the subject. In some embodiments, a regimen of about 25 mg of etanercept is administered to the subject about 3 days after the administration of the modified beta cell or composition to the subject. In some embodiments, a regimen of about 25 mg of etanercept is administered to the subject about 7 days after the administration of the modified beta cell or composition to the subject. In some embodiments, a regimen of about 25 mg of etanercept is administered to the subject about 10 days after the administration of the modified beta cell or composition to the subject. In some embodiments, i) a regimen of about 50 mg of etanercept is administered to the subject on the same day as the administration of the modified beta cell or composition to the subject; and ii) a regimen of about 25 mg etanercept is administered to the subject about 3 days, about 7 days, and about 10 days after the administration of the modified beta cell or composition to the subject. In some embodiments, the etanercept regimen is administered at a lower dose.

In some embodiments, the subject is administered a regimen of etanercept and a regimen of ATG. In some embodiments, the subject is administered at least one regimen of etanercept and at least one regimen of ATG. In some embodiments, the subject is administered the at least one regimen of ATG prior to, on the same day as, concurrent with, and/or after the at least one regimen of etanercept. In some embodiments, the subject is administered the at least one regimen of ATG prior to the at least one regimen of etanercept. In some embodiments, a regimen of about 40 mg/kg of ATG is administered to the subject each day for four consecutive days. In some embodiments, a first regimen of about 25 mg of etanercept is administered to the subject twice a week for two consecutive weeks after the regimen of ATG. In some embodiments, a regimen of about 25 mg of etanercept is administered to the subject once a month for about four months after the first regimen of etanercept. In some embodiments, the etanercept regimen is administered at a lower dose. In some embodiments, the ATG regimen is administered at a lower dose.

In some embodiments, the subject is administered a regimen of etanercept and a regimen of an IL-1 receptor antagonist. In some embodiments, the subject is administered at least one regimen of etanercept and at least one regimen of an IL-1 receptor antagonist. In some embodiments, the subject is administered the at least one regimen of an IL-1 receptor antagonist prior to, on the same day as, concurrent with, and/or after the at least one regimen of etanercept.

e. mTOR Inhibitors

In some aspects, the one or more immunosuppressive agents comprise a mechanistic target of rapamycin (mTOR) inhibitor (e.g., one or more mTOR inhibitors). mTOR inhibitors are drugs that inhibit mTOR, which is a serine/threonine-specific protein kinase that belongs to the family of phosphatidylinositol-3 kinase (PI3K) related kinases (PIKKs). In some embodiments, the mTOR inhibitor is rapamycin, or an analog thereof, such as but not limited to sirolimus, temsirolimus, everolimus, ridaforolimus, umirolimus, or zotarolimus. In some embodiments, the mTOR inhibitor is sirolimus. In some embodiments, the mTOR inhibitor is administered to the subject (e.g. one or more regimens of the mTOR inhibitor is administered to the subject). In some embodiments, the mTOR inhibitor is administered to the subject in one or more compositions, e.g. a pharmaceutical composition containing the mTOR inhibitor. In some embodiments, the mTOR inhibitor is administered to the subject prior to, concurrent with, and/or after the administration of the modified beta cell or composition to the subject. In some embodiments, at least one regimen of the mTOR inhibitor is administered to the subject prior to, concurrent with, and/or after the administration of the modified beta cell or composition to the subject.

In some embodiments, the one or more immunosuppressive agents comprise sirolimus. In some embodiments, the sirolimus (e.g. a regimen of sirolimus) is administered to the subject prior to, concurrent with, and/or after the administration of the modified beta cell or composition to the subject. In some embodiments, at least one regimen of sirolimus is administered to the subject prior to, concurrent with, and/or after the administration of the modified beta cell or composition to the subject.

In some embodiments, the sirolimus (e.g. a regimen of sirolimus) is administered to the subject prior to the administration of the modified beta cell or composition to the subject. In some embodiments, the sirolimus is administered to the subject only prior to the administration of the modified beta cell or composition to the subject. In some embodiments, at least one regimen of sirolimus is administered to the subject prior to the administration of the modified beta cell or composition to the subject. In some embodiments, more than one regimen of sirolimus is administered to the subject prior to administration of the modified beta cell or composition to the subject. In some embodiments, the sirolimus (e.g. at least one regimen of the sirolimus) is administered between about 30 seconds and about 10 weeks prior to administration of the modified beta cell or composition to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks prior to administration of the modified beta cell or composition to the subject. In some embodiments, the sirolimus is administered at least about 30 seconds prior to administration of the modified beta cell or composition to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, or more, prior to administration of the modified beta cell or composition to the subject. In some embodiments, the sirolimus is administered less than about 10 weeks prior to administration of the modified beta cell or composition to the subject, such less than about any of 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the modified beta cell or composition to the subject. In some embodiments, the sirolimus is administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the modified beta cell or composition to the subject.

In some embodiments, the sirolimus (e.g. a regimen of sirolimus, such as a first regimen) is administered to the subject on the same day as the administration of the modified beta cell or composition to the subject. In some embodiments, at least one regimen of sirolimus is administered to the subject on the same day as the administration of the modified beta cell or composition to the subject. In some embodiments, the sirolimus is administered to the subject concurrent with administration of the modified beta cell or composition to the subject. In some embodiments, at least one regimen of sirolimus is administered to the subject concurrent with administration of the modified beta cell or composition to the subject. In some embodiments, the sirolimus is administered to the subject on the same day as administration of the modified beta cell or composition to the subject, and continued to be administered over the course of the subject's lifespan. In some embodiments, the sirolimus is administered to the subject concurrent with administration of the modified beta cell or composition to the subject, and continued to be administered over the course of the subject's lifespan. In some embodiments, a first regimen of the sirolimus is administered to the subject concurrent with administration of the modified beta cell or composition to the subject. In some embodiments, a second regimen the sirolimus is administered to the subject concurrent with administration of the modified beta cell or composition to the subject. In some embodiments, the sirolimus is administered to the subject on the same day as each round of administration of the modified beta cell or composition. In some embodiments, the sirolimus is administered to the subject on the same day as each round of administration of the modified beta cell or composition, and continued to be administered over the course of the subject's lifespan. In some embodiments, the sirolimus is administered to the subject concurrent with each round of administration of the modified beta cell or composition. In some embodiments, the sirolimus is administered to the subject concurrent with each round of administration of the modified beta cell or composition, and continued to be administered over the course of the subject's lifespan.

In some embodiments, the sirolimus (e.g. a regimen of sirolimus) is administered to the subject after administration of the modified beta cell or composition to the subject. In some embodiments, at least one regimen of sirolimus is administered to the subject after administration of the modified beta cell or composition to the subject. In some embodiments, more than one regimen of sirolimus is administered to the subject after administration of the modified beta cell or composition to the subject. In some embodiments, the sirolimus is administered to the subject only after administration of a first and/or second regimen of the modified beta cell or composition to the subject. In some embodiments, the sirolimus is administered between about 30 seconds and about 10 weeks after administration of the modified beta cell or composition to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks after administration of the modified beta cell or composition to the subject. In some embodiments, the sirolimus is administered at least about 30 seconds after administration of the modified beta cell or composition to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, or more, after administration of the modified beta cell or composition to the subject. In some embodiments, the sirolimus is administered less than about 10 weeks after administration of the modified beta cell or composition to the subject, such less than about any of 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the modified beta cell or composition to the subject. In some embodiments, the sirolimus is administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds after administration of the modified beta cell or composition to the subject. In some embodiments, the sirolimus is administered to the subject after administration of the modified beta cell or composition, and continued to be administered over the course of the subject's lifespan. In some embodiments, the sirolimus is administered to the subject after each round of administration of the modified beta cell or composition. In some embodiments, the sirolimus is administered to the subject after each round of administration of the modified beta cell or composition, and continued to be administered over the course of the subject's lifespan. In some embodiments, the sirolimus is administered to the subject each day after the modified beta cell or composition is administered to the subject. In some embodiments, the sirolimus is administered to the subject each day for up to about 3 months after the modified beta cell or composition is administered to the subject.

In some embodiments, the sirolimus is administered to the subject on the same day and after the administration of the modified beta cell or composition is administered to the subject. In some embodiments, the sirolimus is administered to the subject concurrent with and after the administration of the modified beta cell or composition to the subject. In some embodiments, the sirolimus is administered to the subject: i) on the same day; and/or ii) each day for up to about 3 months after the administration of the composition comprising modified beta cell to the subject.

In some embodiments, a regimen of between about 0.05 mg/kg and about 1.0 mg/kg sirolimus is administered to the subject, such as a regimen of between about 0.05 mg/kg and about 0.1 mg/kg, between about 0.1 mg/kg and about 0.5 mg/kg, or between about 0.3 mg/kg and about 1.0 mg/kg. In some embodiments, a regimen of between about 0.1 mg/kg and about 0.2 mg/kg of sirolimus is administered to the subject. In some embodiments, a regimen of greater than about 0.05 mg/kg of sirolimus is administered to the subject, such as a regimen of greater than any of about 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, or greater, of sirolimus. In some embodiments, a regimen of less than about 1.0 mg/kg of sirolimus is administered to the subject, such as a regimen of less than any of about 0.9 mg/kg, 0.8 mg/kg, 0.7 mg/kg, 0.6 mg/kg, 0.5 mg/kg, 0.4 mg/kg, 0.3 mg/kg, 0.2 mg/kg, 0.1 mg/kg, 0.05 mg/kg, or less, of sirolimus. In some embodiments, a regimen of about 0.1 mg/kg sirolimus is administered to the subject. In some embodiments, a regimen of about 0.2 mg/kg sirolimus is administered to the subject. In some embodiments, the sirolimus is administered orally.

In some embodiments, a total daily dosage of sirolimus administered to the subject yields a blood trough level of between about 1 ng/mL and about 30 ng/mL, such as a blood trough level of between about 2 ng/mL and about 25 ng/mL, between about 5 ng/mL and about 20 ng/mL, or between about 10 ng/mL and about 15 ng/mL, inclusive of each. In some embodiments, the total daily dosage of sirolimus administered to the subject yields a blood trough level of between about 12 ng/mL and about 15 ng/mL. In some embodiments, a total daily dosage of sirolimus administered to the subject yields a blood trough level of greater than about 1 ng/mL, such as greater than any of about 5 ng/mL, 10 ng/mL, 15 ng/mL, 20 ng/mL, or greater of sirolimus. In some embodiments, a total daily dosage of sirolimus administered to the subject yields a blood trough level of less than about 20 ng/mL, such as less than any of about 15 ng/mL, 10 ng/mL, 5 ng/mL, 1 ng/mL, or less of sirolimus. In some embodiments, the total daily dosage of sirolimus administered to the subject yields a blood trough level of between about 12 ng/mL and about 15 ng/mL for about 3 months after the administration of the modified beta cell or composition to the subject. In some embodiments, the total daily dosage of sirolimus administered to the subject yields a blood trough level of between about 7 ng/mL and about 10 ng/mL. In some embodiments, the total daily dosage of sirolimus administered to the subject yields a blood trough level of between about 7 ng/mL and about 10 ng/mL after about 3 months after the administration of the modified beta cell or composition to the subject. In some embodiments, the sirolimus is administered orally.

In some embodiments, a regimen of about 0.2 mg/kg of sirolimus is administered to the subject on the same day as the administration of the modified beta cell or composition to the subject. In some embodiments, a regimen of about 0.1 mg/kg of sirolimus is administered to the subject each day after administration of the modified beta cell or composition to the subject. In some embodiments, a regimen of about 0.1 mg/kg of sirolimus is administered to the subject each day for up to about 3 months after administration of the modified beta cell or composition to the subject. In some embodiments, a regimen of about 0.1 mg/kg of sirolimus is administered to the subject each day after about 3 months after administration of the modified beta cell or composition to the subject. In some embodiments, i) a regimen of about 0.2 mg/kg of sirolimus is administered to the subject on the same day as the administration of the modified beta cell or composition; and/or ii) a regimen of about 0.1 mg/kg of sirolimus is administered to the subject each day up to about 3 months after the administration of the modified beta cell or composition. In some embodiments, the sirolimus regimen is administered at a lower dose. In some embodiments, the total daily dosage of sirolimus administered to the subject yields a blood trough level of between about 12 ng/mL and about 15 ng/mL for about 3 months after the administration of the modified beta cell or composition to the subject. In some embodiments, the total daily dosage of sirolimus administered to the subject yields a blood trough level of between about and between about 7 ng/mL and about 10 ng/mL after about 3 months after the administration of the modified beta cell or composition to the subject. In some embodiments, the blood trough level of sirolimus is lower.

f. Calcineurin Inhibitors

In some aspects, the one or more immunosuppressive agents comprise a calcineurin inhibitor (e.g., one or more calcineurin inhibitors). Calcineurin inhibitors are immunosuppressant drugs that inhibit the action of calcineurin, which is an enzyme that activates T-cells of the immune system. These drugs often reduce IL-2 production and IL-2 receptor expression, thus reducing T-cell activation. Exemplary calcineurin inhibitors include, but are not limited to Astagraf XL®, Cequa™, cyclosporine, cyclosporine ophthalmic, Elidel®, Envarsus XR®, Gengraf®, Hecoria, Lupkynis™, Neoral, pimecrolimus, Prograf®, Protopic, Restasis®, Sandimmune®, tacrolimus, tacrolimus ointment, Verkazia®, or voclosporin. In some embodiments, the calcineurin inhibitor is tacrolimus (FK-506). In some embodiments, the calcineurin inhibitor is cyclosporine. In some embodiments, the calcineurin inhibitor is administered to the subject (e.g. one or more regimens of the calcineurin inhibitor is administered to the subject). In some embodiments, the calcineurin inhibitor is administered to the subject in one or more compositions, e.g. a pharmaceutical composition containing the calcineurin inhibitor. In some embodiments, the calcineurin inhibitor is administered to the subject prior to, concurrent with, and/or after the administration of the modified beta cell or composition to the subject. In some embodiments, at least one regimen of the calcineurin inhibitor is administered to the subject prior to, concurrent with, and/or after the administration of the modified beta cell or composition to the subject.

In some embodiments, the one or more immunosuppressive agents comprise tacrolimus. In some embodiments, the tacrolimus (e.g. a regimen of tacrolimus) is administered to the subject prior to, concurrent with, and/or after the administration of the modified beta cell or composition to the subject. In some embodiments, at least one regimen of tacrolimus is administered to the subject prior to, concurrent with, and/or after the administration of the modified beta cell or composition to the subject.

In some embodiments, the tacrolimus (e.g. a regimen of tacrolimus) is administered to the subject prior to the administration of the modified beta cell or composition to the subject. In some embodiments, the tacrolimus is administered to the subject only prior to the administration of the modified beta cell or composition to the subject. In some embodiments, at least one regimen of tacrolimus is administered to the subject prior to the administration of the modified beta cell or composition to the subject. In some embodiments, more than one regimen of tacrolimus is administered to the subject prior to administration of the modified beta cell or composition to the subject. In some embodiments, the tacrolimus (e.g. at least one regimen of the tacrolimus) is administered between about 30 seconds and about 10 weeks prior to administration of the modified beta cell or composition to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks prior to administration of the modified beta cell or composition to the subject. In some embodiments, the tacrolimus is administered at least about 30 seconds prior to administration of the modified beta cell or composition to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, or more, prior to administration of the modified beta cell or composition to the subject. In some embodiments, the tacrolimus is administered less than about 10 weeks prior to administration of the modified beta cell or composition to the subject, such less than about any of 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the modified beta cell or composition to the subject. In some embodiments, the tacrolimus is administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the modified beta cell or composition to the subject.

In some embodiments, the tacrolimus (e.g. a regimen of tacrolimus, such as a first regimen) is administered to the subject on the same day as the administration of the modified beta cell or composition to the subject. In some embodiments, at least one regimen of tacrolimus is administered to the subject on the same day as the administration of the modified beta cell or composition to the subject. In some embodiments, the tacrolimus is administered to the subject concurrent with administration of the modified beta cell or composition to the subject. In some embodiments, at least one regimen of tacrolimus is administered to the subject concurrent with administration of the modified beta cell or composition to the subject. In some embodiments, the tacrolimus is administered to the subject on the same day as administration of the modified beta cell or composition to the subject, and continued to be administered over the course of the subject's lifespan. In some embodiments, the tacrolimus is administered to the subject concurrent with administration of the modified beta cell or composition to the subject, and continued to be administered over the course of the subject's lifespan. In some embodiments, a first regimen of the tacrolimus is administered to the subject concurrent with administration of the modified beta cell or composition to the subject. In some embodiments, a second regimen the tacrolimus is administered to the subject concurrent with administration of the modified beta cell or composition to the subject. In some embodiments, the tacrolimus is administered to the subject on the same day as each round of administration of the modified beta cell or composition. In some embodiments, the tacrolimus is administered to the subject on the same day as each round of administration of the modified beta cell or composition, and continued to be administered over the course of the subject's lifespan. In some embodiments, the tacrolimus is administered to the subject concurrent with each round of administration of the modified beta cell or composition. In some embodiments, the tacrolimus is administered to the subject concurrent with each round of administration of the modified beta cell or composition, and continued to be administered over the course of the subject's lifespan.

In some embodiments, the tacrolimus (e.g. a regimen of tacrolimus) is administered to the subject after administration of the modified beta cell or composition to the subject. In some embodiments, at least one regimen of tacrolimus is administered to the subject after administration of the modified beta cell or composition to the subject. In some embodiments, more than one regimen of tacrolimus is administered to the subject after administration of the modified beta cell or composition to the subject. In some embodiments, the tacrolimus is administered to the subject only after administration of a first and/or second regimen of the modified beta cell or composition to the subject. In some embodiments, the tacrolimus is administered between about 30 seconds and about 10 weeks after administration of the modified beta cell or composition to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks after administration of the modified beta cell or composition to the subject. In some embodiments, the tacrolimus is administered at least about 30 seconds after administration of the modified beta cell or composition to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, or more, after administration of the modified beta cell or composition to the subject. In some embodiments, the tacrolimus is administered less than about 10 weeks after administration of the modified beta cell or composition to the subject, such less than about any of 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the modified beta cell or composition to the subject. In some embodiments, the tacrolimus is administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds after administration of the modified beta cell or composition to the subject. In some embodiments, the tacrolimus is administered to the subject after administration of the modified beta cell or composition, and continued to be administered over the course of the subject's lifespan. In some embodiments, the tacrolimus is administered to the subject after each round of administration of the modified beta cell or composition. In some embodiments, the tacrolimus is administered to the subject after each round of administration of the modified beta cell or composition, and continued to be administered over the course of the subject's lifespan. In some embodiments, the tacrolimus is administered to the subject each day after the modified beta cell or composition is administered to the subject. In some embodiments, the tacrolimus is administered to the subject each day for up to about 3 months after the modified beta cell or composition is administered to the subject.

In some embodiments, a regimen of between about 0.05 mg and about 10 mg tacrolimus is administered to the subject, such as a regimen of between about 0.05 mg and about 1 mg, between about 0.5 mg and about 5 mg, or between about 2.5 mg and about 10 mg. In some embodiments, a regimen of between about 0.1 mg and about 5 mg of tacrolimus is administered to the subject. In some embodiments, a regimen of greater than about 0.05 mg of tacrolimus is administered to the subject, such as a regimen of greater than any of about 0.1 mg, 0.5 mg, 1 mg, 2 mg, 3 mg, 4 mg, 5 mg, 6 mg, 7 mg, 8 mg, 9 mg, 10 mg, or greater, of tacrolimus. In some embodiments, a regimen of less than about 10 mg of tacrolimus is administered to the subject, such as a regimen of less than any of about 9 mg, 8 mg, 7 mg, 6 mg, 5 mg, 4 mg, 3 mg, 2 mg, 1 mg, 0.5 mg, 0.1 mg, or less, of tacrolimus.

In some embodiments, a total daily dosage of tacrolimus administered to the subject yields a blood trough level of between about 1 ng/mL and about 30 ng/mL, such as a blood trough level of between about 2 ng/mL and about 25 ng/mL, between about 5 ng/mL and about 20 ng/mL, or between about 10 ng/mL and about 15 ng/mL, inclusive of each. In some embodiments, the total daily dosage of tacrolimus administered to the subject yields a blood trough level of between about 12 ng/mL and about 15 ng/mL. In some embodiments, a total daily dosage of tacrolimus administered to the subject yields a blood trough level of greater than about 1 ng/mL, such as greater than any of about 5 ng/mL, 10 ng/mL, 15 ng/mL, 20 ng/mL, or greater of tacrolimus. In some embodiments, a total daily dosage of tacrolimus administered to the subject yields a blood trough level of less than about 20 ng/mL, such as less than any of about 15 ng/mL, 10 ng/mL, 5 ng/mL, 1 ng/mL, or less of tacrolimus. In some embodiments, the total daily dosage of tacrolimus administered to the subject yields a blood trough level of between about 5 ng/mL and about 10 ng/mL. In some embodiments, the total daily dosage of tacrolimus administered to the subject yields a blood trough level of between about 10 ng/mL and about 15 ng/mL.

In some embodiments, the one or more immunosuppressive agents comprise cyclosporine. In some embodiments, the cyclosporine (e.g. a regimen of cyclosporine) is administered to the subject prior to, concurrent with, and/or after the administration of the modified beta cell or composition to the subject. In some embodiments, at least one regimen of cyclosporine is administered to the subject prior to, concurrent with, and/or after the administration of the modified beta cell or composition to the subject. In some embodiments, a regimen of cyclosporine is administered to the subject when the subject displays intolerance to a regimen of one or more other immunosuppressive agents.

In some embodiments, the cyclosporine (e.g. a regimen of cyclosporine) is administered to the subject prior to the administration of the modified beta cell or composition to the subject. In some embodiments, the cyclosporine is administered to the subject only prior to the administration of the modified beta cell or composition to the subject. In some embodiments, at least one regimen of cyclosporine is administered to the subject prior to the administration of the modified beta cell or composition to the subject. In some embodiments, more than one regimen of cyclosporine is administered to the subject prior to administration of the modified beta cell or composition to the subject. In some embodiments, the cyclosporine (e.g. at least one regimen of the cyclosporine) is administered between about 30 seconds and about 10 weeks prior to administration of the modified beta cell or composition to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks prior to administration of the modified beta cell or composition to the subject. In some embodiments, the cyclosporine is administered at least about 30 seconds prior to administration of the modified beta cell or composition to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, or more, prior to administration of the modified beta cell or composition to the subject. In some embodiments, the cyclosporine is administered less than about 10 weeks prior to administration of the modified beta cell or composition to the subject, such less than about any of 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the modified beta cell or composition to the subject. In some embodiments, the cyclosporine is administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the modified beta cell or composition to the subject.

In some embodiments, the cyclosporine (e.g. a regimen of cyclosporine, such as a first regimen) is administered to the subject on the same day as the administration of the modified beta cell or composition to the subject. In some embodiments, at least one regimen of cyclosporine is administered to the subject on the same day as the administration of the modified beta cell or composition to the subject. In some embodiments, the cyclosporine is administered to the subject concurrent with administration of the modified beta cell or composition to the subject. In some embodiments, at least one regimen of cyclosporine is administered to the subject concurrent with administration of the modified beta cell or composition to the subject. In some embodiments, the cyclosporine is administered to the subject on the same day as administration of the modified beta cell or composition to the subject, and continued to be administered over the course of the subject's lifespan. In some embodiments, the cyclosporine is administered to the subject concurrent with administration of the modified beta cell or composition to the subject, and continued to be administered over the course of the subject's lifespan. In some embodiments, a first regimen of the cyclosporine is administered to the subject concurrent with administration of the modified beta cell or composition to the subject. In some embodiments, a second regimen the cyclosporine is administered to the subject concurrent with administration of the modified beta cell or composition to the subject. In some embodiments, the cyclosporine is administered to the subject on the same day as each round of administration of the modified beta cell or composition. In some embodiments, the cyclosporine is administered to the subject on the same day as each round of administration of the modified beta cell or composition, and continued to be administered over the course of the subject's lifespan. In some embodiments, the cyclosporine is administered to the subject concurrent with each round of administration of the modified beta cell or composition. In some embodiments, the cyclosporine is administered to the subject concurrent with each round of administration of the modified beta cell or composition, and continued to be administered over the course of the subject's lifespan.

In some embodiments, the cyclosporine (e.g. a regimen of cyclosporine) is administered to the subject after administration of the modified beta cell or composition to the subject. In some embodiments, at least one regimen of cyclosporine is administered to the subject after administration of the modified beta cell or composition to the subject. In some embodiments, more than one regimen of cyclosporine is administered to the subject after administration of the modified beta cell or composition to the subject. In some embodiments, the cyclosporine is administered to the subject only after administration of a first and/or second regimen of the modified beta cell or composition to the subject. In some embodiments, the cyclosporine is administered between about 30 seconds and about 10 weeks after administration of the modified beta cell or composition to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks after administration of the modified beta cell or composition to the subject. In some embodiments, the cyclosporine is administered at least about 30 seconds after administration of the modified beta cell or composition to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, or more, after administration of the modified beta cell or composition to the subject. In some embodiments, the cyclosporine is administered less than about 10 weeks after administration of the modified beta cell or composition to the subject, such less than about any of 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the modified beta cell or composition to the subject. In some embodiments, the cyclosporine is administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds after administration of the modified beta cell or composition to the subject. In some embodiments, the cyclosporine is administered to the subject after administration of the modified beta cell or composition, and continued to be administered over the course of the subject's lifespan. In some embodiments, the cyclosporine is administered to the subject after each round of administration of the modified beta cell or composition. In some embodiments, the cyclosporine is administered to the subject after each round of administration of the modified beta cell or composition, and continued to be administered over the course of the subject's lifespan. In some embodiments, the cyclosporine is administered to the subject each day after the modified beta cell or composition is administered to the subject. In some embodiments, the cyclosporine is administered to the subject each day.

In some embodiments, a regimen of between about 1 mg/kg and about 20 mg/kg cyclosporine is administered to the subject, such as a regimen of between about 1 mg/kg and about 10 mg/kg, between about 5 mg/kg and about 15 mg/kg, or between about 10 mg/kg and about 20 mg/kg. In some embodiments, a regimen of between about 2 mg/kg and about 10 mg/kg of cyclosporine is administered to the subject. In some embodiments, a regimen of between about 10 mg/kg and about 12 mg/kg of cyclosporine is administered to the subject. In some embodiments, a regimen of greater than about 1 mg/kg of cyclosporine is administered to the subject, such as a regimen of greater than any of about 2 mg/kg, 5 mg/kg, 10 mg/kg, 15 mg/kg, 20 mg/kg, or greater, of sirolimus. In some embodiments, a regimen of less than about 20 mg/kg of cyclosporine is administered to the subject, such as a regimen of less than any of about 15 mg/kg, 10 mg/kg, 5 mg/kg, 2 mg/kg, 1 mg/kg, or less, of cyclosporine. In some embodiments, a regimen of about 6 mg/kg cyclosporine is administered to the subject. In some embodiments, a regimen of cyclosporine is administered to the subject each day.

In some embodiments, a total daily dosage of cyclosporine administered to the subject yields a blood trough level of between about 50 ng/mL and about 300 ng/mL, such as a blood trough level of between about 50 ng/mL and about 150 ng/mL, between about 100 ng/mL and about 250 ng/mL, or between about 150 ng/mL and about 300 ng/mL, inclusive of each. In some embodiments, a total daily dosage of cyclosporine administered to the subject yields a blood trough level of greater than about 50 ng/mL, such as greater than any of about 100 ng/mL, 150 ng/mL, 200 ng/mL, 250 ng/mL, 300 ng/mL, or greater of cyclosporine. In some embodiments, a total daily dosage of cyclosporine administered to the subject yields a blood trough level of less than about 300 ng/mL, such as less than any of about 250 ng/mL, 200 ng/mL, 150 ng/mL, 100 ng/mL, 50 ng/mL or less of cyclosporine.

In some embodiments, the subject is administered a regimen of cyclosporine and a regimen of ATG. In some embodiments, the subject is administered at least one regimen of cyclosporine and at least one regimen of ATG. In some embodiments, the subject is administered the at least one regimen of ATG prior to, on the same day as, concurrent with, and/or after the at least one regimen of cyclosporine. In some embodiments, the subject is administered the at least one regimen of ATG prior to the at least one regimen of cyclosporine. In some embodiments, a regimen of about 40 mg/kg of ATG is administered to the subject each day for four consecutive days. In some embodiments, a regimen of between about 10 mg/kg and about 12 mg/kg of cyclosporine is administered to the subject each day for about 6 months after the regimen of ATG is administered to the subject. In some embodiments, the cyclosporine regimen is administered at a lower dose. In some embodiments, the ATG regimen is administered at a lower dose.

g. Inosine-5′-Monophosphate Dehydrogenase (IMPDH) Inhibitors

In some aspects, the one or more immunosuppressive agents comprise an inosine-5′-monophosphate dehydrogenase (IMPDH) inhibitor (e.g., one or more IMPDH inhibitors). IMPDH inhibitors are immunosuppressant drugs that target IMPDH, which is a rate-limiting enzyme involved in guanosine and deoxyguanosine biosynthesis and widely expressed in immunocytes. In some embodiments, the IMPDH inhibitor is mycophenolic acid (MPA), mycophenolate mofetil (MMF), or mycophenolate sodium (MS). In some embodiments, the IMPDH inhibitor is MPA. In some embodiments, the IMPDH inhibitor is MMF. In some embodiments, the IMPDH inhibitor is MS. In some embodiments, the IMPDH inhibitor is administered to the subject (e.g. one or more regimens of the IMPDH inhibitor is administered to the subject). In some embodiments, the IMPDH inhibitor is administered to the subject in one or more compositions, e.g. a pharmaceutical composition containing the IMPDH inhibitor. In some embodiments, the IMPDH inhibitor is administered to the subject prior to, concurrent with, and/or after the administration of the modified beta cell or composition to the subject. In some embodiments, at least one regimen of IMPDH inhibitor is administered to the subject prior to, concurrent with, and/or after the administration of the modified beta cell or composition to the subject.

In some embodiments, the one or more immunosuppressive agents comprise MPA. In some embodiments, the MPA (e.g. a regimen of MPA) is administered to the subject prior to, concurrent with, and/or after the administration of the modified beta cell or composition to the subject. In some embodiments, at least one regimen of MPA is administered to the subject prior to, concurrent with, and/or after the administration of the modified beta cell or composition to the subject.

In some embodiments, the MPA (e.g. a regimen of MPA) is administered to the subject prior to the administration of the modified beta cell or composition to the subject. In some embodiments, the MPA is administered to the subject only prior to the administration of the modified beta cell or composition to the subject. In some embodiments, at least one regimen of MPA is administered to the subject prior to the administration of the modified beta cell or composition to the subject. In some embodiments, more than one regimen of MPA is administered to the subject prior to administration of the modified beta cell or composition to the subject. In some embodiments, the MPA (e.g. at least one regimen of the MPA) is administered between about 30 seconds and about 10 weeks prior to administration of the modified beta cell or composition to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks prior to administration of the composition comprising a modified beta cell to the subject. In some embodiments, the MPA is administered at least about 30 seconds prior to administration of the composition comprising a modified beta cell to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, or more, prior to administration of the composition comprising a modified beta cell to the subject. In some embodiments, the MPA is administered less than about 10 weeks prior to administration of the composition comprising a modified beta cell to the subject, such less than about any of 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the composition comprising a modified beta cell to the subject. In some embodiments, the MPA is administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the composition comprising a modified beta cell to the subject.

In some embodiments, the MPA (e.g. a regimen of MPA, such as a first regimen) is administered to the subject on the same day as the administration of the composition comprising a modified beta cell to the subject. In some embodiments, at least one regimen of MPA is administered to the subject on the same day as the administration of the composition comprising a modified beta cell to the subject. In some embodiments, the MPA is administered to the subject concurrent with administration of the composition comprising a modified beta cell to the subject. In some embodiments, at least one regimen of MPA is administered to the subject concurrent with administration of the composition comprising a modified beta cell to the subject. In some embodiments, the MPA is administered to the subject on the same day as administration of the composition comprising a modified beta cell to the subject, and continued to be administered over the course of the subject's lifespan. In some embodiments, the MPA is administered to the subject concurrent with administration of the composition comprising a modified beta cell to the subject, and continued to be administered over the course of the subject's lifespan. In some embodiments, a first regimen of the MPA is administered to the subject concurrent with administration of the composition comprising a modified beta cell to the subject. In some embodiments, a second regimen the MPA is administered to the subject concurrent with administration of the composition comprising a modified beta cell to the subject. In some embodiments, the MPA is administered to the subject on the same day as each round of administration of the composition comprising a modified beta cell. In some embodiments, the MPA is administered to the subject on the same day as each round of administration of the composition comprising a modified beta cell, and continued to be administered over the course of the subject's lifespan. In some embodiments, the MPA is administered to the subject concurrent with each round of administration of the composition comprising a modified beta cell. In some embodiments, the MPA is administered to the subject concurrent with each round of administration of the composition comprising a modified beta cell, and continued to be administered over the course of the subject's lifespan.

In some embodiments, the MPA (e.g. a regimen of MPA) is administered to the subject after administration of the composition comprising a modified beta cell to the subject. In some embodiments, at least one regimen of MPA is administered to the subject after administration of the composition comprising a modified beta cell to the subject. In some embodiments, more than one regimen of MPA is administered to the subject after administration of the composition comprising a modified beta cell to the subject. In some embodiments, the MPA is administered to the subject only after administration of a first and/or second regimen of the composition comprising a modified beta cell to the subject. In some embodiments, the MPA is administered between about 30 seconds and about 10 weeks after administration of the composition comprising a modified beta cell to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks after administration of the composition comprising a modified beta cell to the subject. In some embodiments, the MPA is administered at least about 30 seconds after administration of the composition comprising a modified beta cell to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, or more, after administration of the composition comprising a modified beta cell to the subject. In some embodiments, the MPA is administered less than about 10 weeks after administration of the composition comprising a modified beta cell to the subject, such less than about any of 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the composition comprising a modified beta cell to the subject. In some embodiments, the MPA is administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds after administration of the composition comprising a modified beta cell to the subject. In some embodiments, the MPA is administered to the subject after administration of the composition comprising a modified beta cell, and continued to be administered over the course of the subject's lifespan. In some embodiments, the MPA is administered to the subject after each round of administration of the composition comprising a modified beta cell. In some embodiments, the MPA is administered to the subject after each round of administration of the composition comprising a modified beta cell, and continued to be administered over the course of the subject's lifespan. In some embodiments, the MPA is administered to the subject each day after the composition comprising a modified beta cell is administered to the subject. In some embodiments, the MPA is administered to the subject each day for up to about 3 months after the composition comprising a modified beta cell is administered to the subject.

In some embodiments, the subject is administered a regimen of MPA and a regimen of tacrolimus. In some embodiments, the subject is administered at least one regimen of MPA and at least one regimen of tacrolimus. In some embodiments, the subject is administered the at least one regimen of tacrolimus prior to, on the same day as, concurrent with, and/or after the at least one regimen of MPA. In some embodiments, the subject is administered the at least one regimen of MPA prior to, on the same day as, concurrent with, and/or after the at least one regimen of tacrolimus.

In some embodiments, the subject is administered a regimen of MPA and a regimen of cyclosporine. In some embodiments, the subject is administered at least one regimen of MPA and at least one regimen of cyclosporine. In some embodiments, the subject is administered the at least one regimen of cyclosporine prior to, on the same day as, concurrent with, and/or after the at least one regimen of MPA. In some embodiments, the subject is administered the at least one regimen of MPA prior to, on the same day as, concurrent with, and/or after the at least one regimen of cyclosporine.

In some embodiments, the MPA is MMF. In some aspects, the one or more immunosuppressive agents comprise MMF. In some embodiments, the MMF (e.g. a regimen of MMF) is administered to the subject prior to, concurrent with, and/or after the administration of the composition comprising a modified beta cell to the subject. In some embodiments, at least one regimen of MMF is administered to the subject prior to, concurrent with, and/or after the administration of the composition comprising a modified beta cell to the subject.

In some embodiments, the MMF (e.g. a regimen of MMF) is administered to the subject prior to the administration of the composition comprising a modified beta cell to the subject. In some embodiments, the MMF is administered to the subject only prior to the administration of the composition comprising a modified beta cell to the subject. In some embodiments, at least one regimen of MMF is administered to the subject prior to the administration of the composition comprising a modified beta cell to the subject. In some embodiments, more than one regimen of MMF is administered to the subject prior to administration of the composition comprising a modified beta cell to the subject. In some embodiments, the MMF (e.g. at least one regimen of the MMF) is administered between about 30 seconds and about 10 weeks prior to administration of the composition comprising a modified beta cell to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks prior to administration of the composition comprising a modified beta cell to the subject. In some embodiments, the MMF is administered at least about 30 seconds prior to administration of the composition comprising a modified beta cell to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, or more, prior to administration of the composition comprising a modified beta cell to the subject. In some embodiments, the MMF is administered less than about 10 weeks prior to administration of the composition comprising a modified beta cell to the subject, such less than about any of 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the composition comprising a modified beta cell to the subject. In some embodiments, the MMF is administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the composition comprising a modified beta cell to the subject.

In some embodiments, the MMF (e.g. a regimen of MMF, such as a first regimen) is administered to the subject on the same day as the administration of the composition comprising a modified beta cell to the subject. In some embodiments, at least one regimen of MMF is administered to the subject on the same day as the administration of the composition comprising a modified beta cell to the subject. In some embodiments, the MMF is administered to the subject concurrent with administration of the composition comprising a modified beta cell to the subject. In some embodiments, at least one regimen of MMF is administered to the subject concurrent with administration of the composition comprising a modified beta cell to the subject. In some embodiments, the MMF is administered to the subject on the same day as administration of the composition comprising a modified beta cell to the subject, and continued to be administered over the course of the subject's lifespan. In some embodiments, the MMF is administered to the subject concurrent with administration of the composition comprising a modified beta cell to the subject, and continued to be administered over the course of the subject's lifespan. In some embodiments, a first regimen of the MMF is administered to the subject concurrent with administration of the composition comprising a modified beta cell to the subject. In some embodiments, a second regimen the MMF is administered to the subject concurrent with administration of the composition comprising a modified beta cell to the subject. In some embodiments, the MMF is administered to the subject on the same day as each round of administration of the composition comprising a modified beta cell. In some embodiments, the MMF is administered to the subject on the same day as each round of administration of the composition comprising a modified beta cell, and continued to be administered over the course of the subject's lifespan. In some embodiments, the MMF is administered to the subject concurrent with each round of administration of the composition comprising a modified beta cell. In some embodiments, the MMF is administered to the subject concurrent with each round of administration of the composition comprising a modified beta cell, and continued to be administered over the course of the subject's lifespan.

In some embodiments, the MMF (e.g. a regimen of MMF) is administered to the subject after administration of the composition comprising a modified beta cell to the subject. In some embodiments, at least one regimen of MMF is administered to the subject after administration of the composition comprising a modified beta cell to the subject. In some embodiments, more than one regimen of MMF is administered to the subject after administration of the composition comprising a modified beta cell to the subject. In some embodiments, the MMF is administered to the subject only after administration of a first and/or second regimen of the composition comprising a modified beta cell to the subject. In some embodiments, the MMF is administered between about 30 seconds and about 10 weeks after administration of the composition comprising a modified beta cell to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks after administration of the composition comprising a modified beta cell to the subject. In some embodiments, the MMF is administered at least about 30 seconds after administration of the composition comprising a modified beta cell to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, or more, after administration of the composition comprising a modified beta cell to the subject. In some embodiments, the MMF is administered less than about 10 weeks after administration of the composition comprising a modified beta cell to the subject, such less than about any of 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the composition comprising a modified beta cell to the subject. In some embodiments, the MMF is administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds after administration of the composition comprising a modified beta cell to the subject. In some embodiments, the MMF is administered to the subject after administration of the composition comprising a modified beta cell, and continued to be administered over the course of the subject's lifespan. In some embodiments, the MMF is administered to the subject after each round of administration of the composition comprising a modified beta cell. In some embodiments, the MMF is administered to the subject after each round of administration of the composition comprising a modified beta cell, and continued to be administered over the course of the subject's lifespan. In some embodiments, the MMF is administered to the subject each day after the composition comprising a modified beta cell is administered to the subject.

In some embodiments, the total daily dose of MMF administered to the subject is between about 10 mg and about 3,000 mg, such as between about 10 mg and about 500 mg, between about 100 mg and about 1,500 mg, or between about 1,000 mg and about 3,000 mg. In some embodiments, the total daily dose of MMF administered to the subject is between about 100 mg and about 2,500 mg. In some embodiments, the total daily dose of MMF administered to the subject is greater than about 10 mg, such as greater than any of about 20 mg, 30 mg, 40 mg, 50 mg, 100 mg, 500 mg, 1,000 mg, 2,000 mg, 3,000 mg, or greater, of MMF. In some embodiments, the total daily dose of MMF administered to the subject is less than about 3,000 mg, such as less than any of about 2,000 mg, 1,000 mg, 500 mg, 100 mg, 50 mg, 40 mg, 30 mg, 20 mg, 10 mg, or less, of MMF. In some embodiments, the total daily dose of MMF administered to the subject is about 100 mg, 500 mg, 1,000 mg, about 1,500 mg, about 2,000 mg, or about 2,500 mg.

In some embodiments, the MPA is MS. In some aspects, the one or more immunosuppressive agents comprise MS. In some embodiments, the MS (e.g. a regimen of MS) is administered to the subject prior to, concurrent with, and/or after the administration of the composition comprising a modified beta cell to the subject. In some embodiments, at least one regimen of MS is administered to the subject prior to, concurrent with, and/or after the administration of the composition comprising a modified beta cell to the subject.

In some embodiments, the MS (e.g. a regimen of MS) is administered to the subject prior to the administration of the composition comprising a modified beta cell to the subject. In some embodiments, the MS is administered to the subject only prior to the administration of the composition comprising a modified beta cell to the subject. In some embodiments, at least one regimen of MS is administered to the subject prior to the administration of the composition comprising a modified beta cell to the subject. In some embodiments, more than one regimen of MS is administered to the subject prior to administration of the composition comprising a modified beta cell to the subject. In some embodiments, the MS (e.g. at least one regimen of the MS) is administered between about 30 seconds and about 10 weeks prior to administration of the composition comprising a modified beta cell to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks prior to administration of the composition comprising a modified beta cell to the subject. In some embodiments, the MS is administered at least about 30 seconds prior to administration of the composition comprising a modified beta cell to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, or more, prior to administration of the composition comprising a modified beta cell to the subject. In some embodiments, the MS is administered less than about 10 weeks prior to administration of the composition comprising a modified beta cell to the subject, such less than about any of 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the composition comprising a modified beta cell to the subject. In some embodiments, the MS is administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the composition comprising a modified beta cell to the subject.

In some embodiments, the MS (e.g. a regimen of MS, such as a first regimen) is administered to the subject on the same day as the administration of the composition comprising a modified beta cell to the subject. In some embodiments, at least one regimen of MS is administered to the subject on the same day as the administration of the composition comprising a modified beta cell to the subject. In some embodiments, the MS is administered to the subject concurrent with administration of the composition comprising a modified beta cell to the subject. In some embodiments, at least one regimen of MS is administered to the subject concurrent with administration of the composition comprising a modified beta cell to the subject. In some embodiments, the MS is administered to the subject on the same day as administration of the composition comprising a modified beta cell to the subject, and continued to be administered over the course of the subject's lifespan. In some embodiments, the MS is administered to the subject concurrent with administration of the composition comprising a modified beta cell to the subject, and continued to be administered over the course of the subject's lifespan. In some embodiments, a first regimen of the MS is administered to the subject concurrent with administration of the composition comprising a modified beta cell to the subject. In some embodiments, a second regimen the MS is administered to the subject concurrent with administration of the composition comprising a modified beta cell to the subject. In some embodiments, the MS is administered to the subject on the same day as each round of administration of the composition comprising a modified beta cell. In some embodiments, the MS is administered to the subject on the same day as each round of administration of the composition comprising a modified beta cell, and continued to be administered over the course of the subject's lifespan. In some embodiments, the MS is administered to the subject concurrent with each round of administration of the composition comprising a modified beta cell. In some embodiments, the MS is administered to the subject concurrent with each round of administration of the composition comprising a modified beta cell, and continued to be administered over the course of the subject's lifespan.

In some embodiments, the MS (e.g. a regimen of MS) is administered to the subject after administration of the composition comprising a modified beta cell to the subject. In some embodiments, at least one regimen of MS is administered to the subject after administration of the composition comprising a modified beta cell to the subject. In some embodiments, more than one regimen of MS is administered to the subject after administration of the composition comprising a modified beta cell to the subject. In some embodiments, the MS is administered to the subject only after administration of a first and/or second regimen of the composition comprising a modified beta cell to the subject. In some embodiments, the MS is administered between about 30 seconds and about 10 weeks after administration of the composition comprising a modified beta cell to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks after administration of the composition comprising a modified beta cell to the subject. In some embodiments, the MS is administered at least about 30 seconds after administration of the composition comprising a modified beta cell to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, or more, after administration of the composition comprising a modified beta cell to the subject. In some embodiments, the MS is administered less than about 10 weeks after administration of the composition comprising a modified beta cell to the subject, such less than about any of 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the composition comprising a modified beta cell to the subject. In some embodiments, the MS is administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds after administration of the composition comprising a modified beta cell to the subject. In some embodiments, the MS is administered to the subject after administration of the composition comprising a modified beta cell, and continued to be administered over the course of the subject's lifespan. In some embodiments, the MS is administered to the subject after each round of administration of the composition comprising a modified beta cell. In some embodiments, the MS is administered to the subject after each round of administration of the composition comprising a modified beta cell, and continued to be administered over the course of the subject's lifespan. In some embodiments, the MS is administered to the subject each day after the composition comprising a modified beta cell is administered to the subject.

In some embodiments, the total daily dose of MS administered to the subject is between about 10 mg and about 2,700 mg, such as between about 10 mg and about 500 mg, between about 100 mg and about 1,500 mg, or between about 1,000 mg and about 2,700 mg. In some embodiments, the total daily dose of MS administered to the subject is between about 100 mg and about 2,500 mg. In some embodiments, the total daily dose of MS administered to the subject is greater than about 10 mg, such as greater than any of about 20 mg, 30 mg, 40 mg, 50 mg, 100 mg, 500 mg, 1,000 mg, 2,000 mg, 2,700 mg, or greater, of MS. In some embodiments, the total daily dose of MS administered to the subject is less than about 2,700 mg, such as less than any of about 2,000 mg, 1,000 mg, 500 mg, 100 mg, 50 mg, 40 mg, 30 mg, 20 mg, 10 mg, or less, of MS. In some embodiments, the total daily dose of MS administered to the subject is about 100 mg, 360 mg, 720 mg, about 1,080 mg, or about 1,440 mg.

h. Antibodies

In some aspects, the one or more immunosuppressive agents comprise an antibody (e.g., one or more antibodies). In some embodiments, the one or more immunosuppressive agents comprise an antibody for binding to MHC, CD2, CD3, CD4, CD7, CD28, B7, CD25, CD40, CD45, CD95, IFN-gamma, TNF-alpha, IL-2Ralpha, IL-4, IL-5, IL-6R, IL-6, IGF, IGFR1, IL-7, IL-8, IL-10, IL-33, CD11 alpha, or CD58, and antibodies binding to any of their ligands. In some embodiments, the one or more immunosuppressive agents comprise an anti-CD3 antibody. In some embodiments, the anti-CD3 antibody is an anti-CD3e antibody. In some embodiments, the anti-CD3 antibody is OKT3. In some embodiments, the one or more immunosuppressive agents comprise an anti-CD95 antibody. In some embodiments, the one or more immunosuppressive agents comprise an anti-IL-33 antibody. In some embodiments, the one or more immunosuppressive agents comprise soluble IL-15R, IL-10, B7 molecules such as B7-1, B7-2, variants thereof, and fragments thereof, ICOS, and OX40. In some embodiments, the one or more immunosuppressive agents comprise an inhibitor of a negative T cell regulator, such as an antibody against CTLA-4, or similar agents. In some embodiments, the antibody is an anti-CD25 antibody. In some embodiments, the antibody is an anti-IL-2Ralpha antibody. In some embodiments, the anti-CD25 antibody or the anti-IL-2Ralpha antibody is basiliximab, daclizumab, or alemtuzumab. In some embodiments, the antibody is basiliximab. In some embodiments, the antibody is daclizumab. In some embodiments, the antibody is alemtuzumab. In some embodiments, the antibody is administered to the subject (e.g. one or more regimens of the antibody is administered to the subject). In some embodiments, the antibody is administered to the subject in one or more compositions, e.g. a pharmaceutical composition containing the antibody. In some embodiments, the antibody is administered to the subject prior to, concurrent with, and/or after the administration of the composition comprising a modified beta cell to the subject. In some embodiments, at least one regimen of antibody is administered to the subject prior to, concurrent with, and/or after the administration of the composition comprising a modified beta cell to the subject.

In some embodiments, the one or more immunosuppressive agents comprise basiliximab. In some embodiments, the basiliximab (e.g. a regimen of basiliximab) is administered to the subject prior to, concurrent with, and/or after the administration of the composition comprising a modified beta cell to the subject. In some embodiments, at least one regimen of basiliximab is administered to the subject prior to, concurrent with, and/or after the administration of the composition comprising a modified beta cell to the subject.

In some embodiments, the basiliximab (e.g. a regimen of basiliximab) is administered to the subject prior to the administration of the composition comprising a modified beta cell to the subject. In some embodiments, the basiliximab is administered to the subject only prior to the administration of the composition comprising a modified beta cell to the subject. In some embodiments, at least one regimen of basiliximab is administered to the subject prior to the administration of the composition comprising a modified beta cell to the subject. In some embodiments, more than one regimen of basiliximab is administered to the subject prior to administration of the composition comprising a modified beta cell to the subject. In some embodiments, the basiliximab (e.g. at least one regimen of basiliximab) is administered between about 30 seconds and about 10 weeks prior to administration of the composition comprising a modified beta cell to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks prior to administration of the composition comprising a modified beta cell to the subject. In some embodiments, the basiliximab is administered at least about 30 seconds prior to administration of the composition comprising a modified beta cell to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, or more, prior to administration of the composition comprising a modified beta cell to the subject. In some embodiments, the basiliximab is administered less than about 10 weeks prior to administration of the composition comprising a modified beta cell to the subject, such less than about any of 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the composition comprising a modified beta cell to the subject. In some embodiments, the basiliximab is administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the composition comprising a modified beta cell to the subject.

In some embodiments, the basiliximab (e.g. a regimen of basiliximab, such as a first regimen) is administered to the subject on the same day as the administration of the composition comprising a modified beta cell to the subject. In some embodiments, at least one regimen of basiliximab is administered to the subject on the same day as the administration of the composition comprising a modified beta cell to the subject. In some embodiments, the basiliximab is administered to the subject concurrent with administration of the composition comprising a modified beta cell to the subject. In some embodiments, at least one regimen of basiliximab is administered to the subject concurrent with administration of the composition comprising a modified beta cell to the subject. In some embodiments, the basiliximab is administered to the subject on the same day as administration of the composition comprising a modified beta cell to the subject, and continued to be administered over the course of the subject's lifespan. In some embodiments, the basiliximab is administered to the subject concurrent with administration of the composition comprising a modified beta cell to the subject, and continued to be administered over the course of the subject's lifespan. In some embodiments, a first regimen of the basiliximab is administered to the subject concurrent with administration of the composition comprising a modified beta cell to the subject. In some embodiments, a second regimen the basiliximab is administered to the subject concurrent with administration of the composition comprising a modified beta cell to the subject. In some embodiments, the basiliximab is administered to the subject on the same day as each round of administration of the composition comprising a modified beta cell. In some embodiments, the basiliximab is administered to the subject on the same day as each round of administration of the composition comprising a modified beta cell, and continued to be administered over the course of the subject's lifespan. In some embodiments, the basiliximab is administered to the subject concurrent with each round of administration of the composition comprising a modified beta cell. In some embodiments, the basiliximab is administered to the subject concurrent with each round of administration of the composition comprising a modified beta cell, and continued to be administered over the course of the subject's lifespan.

In some embodiments, the basiliximab (e.g. a regimen of basiliximab) is administered to the subject after administration of the composition comprising a modified beta cell to the subject. In some embodiments, at least one regimen of basiliximab is administered to the subject after administration of the composition comprising a modified beta cell to the subject. In some embodiments, more than one regimen of basiliximab is administered to the subject after administration of the composition comprising a modified beta cell to the subject. In some embodiments, the basiliximab is administered to the subject only after administration of a first and/or second regimen of the composition comprising a modified beta cell to the subject. In some embodiments, the basiliximab is administered between about 30 seconds and about 60 months after administration of the composition comprising a modified beta cell to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks, or between about 10 weeks and about 60 months, after administration of the composition comprising a modified beta cell to the subject. In some embodiments, the basiliximab is administered at least about 30 seconds after administration of the composition comprising a modified beta cell to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 12 months, 24 months, 60 months, or more, after administration of the composition comprising a modified beta cell to the subject. In some embodiments, the basiliximab is administered less than about 60 months after administration of the composition comprising a modified beta cell to the subject, such less than about any of 24 months, 12 months, 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the composition comprising a modified beta cell to the subject. In some embodiments, the basiliximab is administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds after administration of the composition comprising a modified beta cell to the subject. In some embodiments, a regimen of basiliximab is administered to the subject about 4 days after the administration of the composition comprising a modified beta cell to the subject. In some embodiments, the basiliximab is administered to the subject after administration of the composition comprising a modified beta cell, and continued to be administered over the course of the subject's lifespan. In some embodiments, the basiliximab is administered to the subject after each round of administration of the composition comprising a modified beta cell. In some embodiments, the basiliximab is administered to the subject after each round of administration of the composition comprising a modified beta cell, and continued to be administered over the course of the subject's lifespan. In some embodiments, the basiliximab is administered to the subject each day after the composition comprising a modified beta cell is administered to the subject.

In some embodiments, a regimen and/or total daily dose of between about 1 mg and about 50 mg basiliximab is administered to the subject, such as a regimen of between about 1 mg and about 10 mg basiliximab, between about 20 mg and about 40 mg, or between about 30 mg and about 50 mg. In some embodiments, a regimen of between about 10 mg and about 30 mg of basiliximab is administered to the subject. In some embodiments, a regimen of greater than about 1 mg of basiliximab is administered to the subject, such as a regimen of greater than any of about 2 mg, 3 mg, 4 mg, 5 mg, 10 mg, 20 mg, 30 mg, 40 mg, 50 mg or greater, of basiliximab. In some embodiments, a regimen of less than about 50 mg of basiliximab is administered to the subject, such as a regimen of less than any of about 40 mg, 30 mg, 20 mg, 10 mg, 5 mg, 4 mg, 3 mg, 2 mg, 1 mg, or less, of basiliximab. In some embodiments, a regimen of about 20 mg basiliximab is administered to the subject.

In some embodiments, a regimen of about 20 mg of basiliximab is administered to the subject on the same day as the administration of the composition comprising a modified beta cell to the subject. In some embodiments, a regimen of about 20 mg of basiliximab is administered to the subject after the administration of the composition comprising a modified beta cell to the subject. In some embodiments, a regimen of about 20 mg of basiliximab is administered to the subject about 4 days after the administration of the composition comprising a modified beta cell to the subject. In some embodiments, i) a regimen of about 20 mg of basiliximab is administered to the subject on the same day as the administration of the modified beta cell to the subject; and ii) a regimen of about 20 mg of basiliximab is administered to the subject about 4 days after the administration of the modified beta cell to the subject. In some embodiments, the regimen of basiliximab is administered at a lower dose.

In some embodiments, the subject is administered a regimen of basiliximab and a regimen of ATG. In some embodiments, the subject is administered at least one regimen of basiliximab and at least one regimen of ATG. In some embodiments, the subject is administered the at least one regimen of ATG prior to, on the same day as, concurrent with, and/or after the at least one regimen of basiliximab. In some embodiments, the subject is administered the at least one regimen of basiliximab prior to, on the same day as, concurrent with, and/or after the at least one regimen of ATG. In some embodiments, the subject is administered the at least one regimen of basiliximab after the at least one regimen of ATG.

In some embodiments, the one or more immunosuppressive agents comprise daclizumab. In some embodiments, the daclizumab (e.g. a regimen of daclizumab) is administered to the subject prior to, concurrent with, and/or after the administration of the composition comprising a modified beta cell to the subject. In some embodiments, at least one regimen of daclizumab is administered to the subject prior to, concurrent with, and/or after the administration of the composition comprising a modified beta cell to the subject.

In some embodiments, the daclizumab (e.g. a regimen of daclizumab) is administered to the subject prior to the administration of the composition comprising a modified beta cell to the subject. In some embodiments, the daclizumab is administered to the subject only prior to the administration of the composition comprising a modified beta cell to the subject. In some embodiments, at least one regimen of daclizumab is administered to the subject prior to the administration of the composition comprising a modified beta cell to the subject. In some embodiments, more than one regimen of daclizumab is administered to the subject prior to administration of the composition comprising a modified beta cell to the subject. In some embodiments, the daclizumab (e.g. at least one regimen of daclizumab) is administered between about 30 seconds and about 10 weeks prior to administration of the composition comprising a modified beta cell to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks prior to administration of the composition comprising a modified beta cell to the subject. In some embodiments, the daclizumab is administered at least about 30 seconds prior to administration of the composition comprising a modified beta cell to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, or more, prior to administration of the composition comprising a modified beta cell to the subject. In some embodiments, the daclizumab is administered less than about 10 weeks prior to administration of the composition comprising a modified beta cell to the subject, such less than about any of 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the composition comprising a modified beta cell to the subject. In some embodiments, the daclizumab is administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the composition comprising a modified beta cell to the subject.

In some embodiments, the daclizumab (e.g. a regimen of daclizumab, such as a first regimen) is administered to the subject on the same day as the administration of the composition comprising a modified beta cell to the subject. In some embodiments, at least one regimen of daclizumab is administered to the subject on the same day as the administration of the composition comprising a modified beta cell to the subject. In some embodiments, the daclizumab is administered to the subject concurrent with administration of the composition comprising a modified beta cell to the subject. In some embodiments, at least one regimen of daclizumab is administered to the subject concurrent with administration of the composition comprising a modified beta cell to the subject. In some embodiments, the daclizumab is administered to the subject on the same day as administration of the composition comprising a modified beta cell to the subject, and continued to be administered over the course of the subject's lifespan. In some embodiments, the daclizumab is administered to the subject concurrent with administration of the composition comprising a modified beta cell to the subject, and continued to be administered over the course of the subject's lifespan. In some embodiments, a first regimen of the daclizumab is administered to the subject concurrent with administration of the composition comprising a modified beta cell to the subject. In some embodiments, a second regimen the daclizumab is administered to the subject concurrent with administration of the composition comprising a modified beta cell to the subject. In some embodiments, the daclizumab is administered to the subject on the same day as each round of administration of the composition comprising a modified beta cell. In some embodiments, the daclizumab is administered to the subject on the same day as each round of administration of the composition comprising a modified beta cell, and continued to be administered over the course of the subject's lifespan. In some embodiments, the daclizumab is administered to the subject concurrent with each round of administration of the composition comprising a modified beta cell. In some embodiments, the daclizumab is administered to the subject concurrent with each round of administration of the composition comprising a modified beta cell, and continued to be administered over the course of the subject's lifespan.

In some embodiments, the daclizumab (e.g. a regimen of daclizumab) is administered to the subject after administration of the composition comprising a modified beta cell to the subject. In some embodiments, at least one regimen of daclizumab is administered to the subject after administration of the composition comprising a modified beta cell to the subject. In some embodiments, more than one regimen of daclizumab is administered to the subject after administration of the composition comprising a modified beta cell to the subject. In some embodiments, the daclizumab is administered to the subject only after administration of a first and/or second regimen of the composition comprising a modified beta cell to the subject. In some embodiments, the daclizumab is administered between about 30 seconds and about 60 months after administration of the composition comprising a modified beta cell to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks, or between about 10 weeks and about 60 months, after administration of the composition comprising a modified beta cell to the subject. In some embodiments, the daclizumab is administered at least about 30 seconds after administration of the composition comprising a modified beta cell to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 12 months, 24 months, 60 months, or more, after administration of the composition comprising a modified beta cell to the subject. In some embodiments, the daclizumab is administered less than about 60 months after administration of the composition comprising a modified beta cell to the subject, such less than about any of 24 months, 12 months, 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the composition comprising a modified beta cell to the subject. In some embodiments, the daclizumab is administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds after administration of the composition comprising a modified beta cell to the subject. In some embodiments, a regimen of basiliximab is administered to the subject about 14 days after the administration of the composition comprising a modified beta cell to the subject. In some embodiments, the daclizumab is administered to the subject after administration of the composition comprising a modified beta cell, and continued to be administered over the course of the subject's lifespan. In some embodiments, the daclizumab is administered to the subject after each round of administration of the composition comprising a modified beta cell. In some embodiments, the daclizumab is administered to the subject after each round of administration of the composition comprising a modified beta cell, and continued to be administered over the course of the subject's lifespan. In some embodiments, the daclizumab is administered to the subject each day after the composition comprising a modified beta cell is administered to the subject.

In some embodiments, a regimen of between about 0.05 mg/kg and about 3.0 mg/kg daclizumab is administered to the subject, such as a regimen of between about 0.05 mg/kg and about 0.5 mg/kg, between about 0.1 mg/kg and about 1.5 mg/kg, or between about 1.0 mg/kg and about 3.0 mg/kg. In some embodiments, a regimen of between about 0.5 mg/kg and about 2.0 mg/kg of daclizumab is administered to the subject. In some embodiments, a regimen of greater than about 0.05 mg/kg of sirolimus is administered to the subject, such as a regimen of greater than any of about 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 2.0 mg/kg, 3.0 mg/kg, or greater, of daclizumab. In some embodiments, a regimen of less than about 3.0 mg/kg of daclizumab is administered to the subject, such as a regimen of less than any of about 2.0 mg/kg, 1.0 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, 0.1 mg/kg, 0.05 mg/kg, or less, of daclizumab. In some embodiments, a regimen of about 1 mg/kg daclizumab is administered to the subject.

In some embodiments, the subject is administered a regimen of daclizumab and a regimen of sirolimus. In some embodiments, the subject is administered a regimen of daclizumab and a regimen of tacrolimus. In some embodiments, the subject is administered a regimen of tacrolimus and a regimen of sirolimus. In some embodiments, the subject is administered at least one regimen of daclizumab and at least one regimen of sirolimus. In some embodiments, the subject is administered at least one regimen of daclizumab and at least one regimen of tacrolimus. In some embodiments, the subject is administered at least one regimen of tacrolimus and at least one regimen of sirolimus. In some embodiments, the subject is administered a regimen of sirolimus on the same day the administration of the composition comprising a modified beta cell. In some embodiments, the subject is administered a regimen of sirolimus after the administration of the composition comprising a modified beta cell. In some embodiments, the subject is administered a regimen of sirolimus each day after the administration of the composition comprising a modified beta cell. In some embodiments, the subject is administered a regimen of tacrolimus on the same day the administration of the composition comprising a modified beta cell. In some embodiments, the subject is administered a regimen of tacrolimus after the administration of the composition comprising a modified beta cell. In some embodiments, the subject is administered a regimen of tacrolimus twice a day about 12 hours after the administration of the composition comprising a modified beta cell. In some embodiments, the subject is administered a regimen of daclizumab after the administration of the composition comprising a modified beta cell. In some embodiments, the subject is administered a regimen of daclizumab about every 14 days after the administration of the composition comprising a modified beta cell. In some embodiments, i) a regimen of about 0.2 mg/kg of sirolimus is administered to the subject on the same day as the administration of the composition comprising a modified beta cell to the subject; ii) a regimen of about 0.1 mg/kg of sirolimus is administered to the subject each day after the administration of the composition comprising a modified beta cell to the subject; iii) a regimen of about 1 mg of tacrolimus is administered to the subject on the same day as the administration of the composition comprising a modified beta cell to the subject; iv) a regimen of about 1 mg of tacrolimus is administered to the subject twice a day about 12 hours after the administration of the composition comprising a modified beta cell to the subject; and v) a regimen of about 1 mg/kg of daclizumab is administered to the subject about every 14 days after the administration of the composition comprising a modified beta cells to the subject. In some embodiments, the total daily dosage of sirolimus administered to the subject yields a blood trough level of between about 12 ng/mL and about 15 ng/mL, inclusive of each, for the first three months after the administration of the composition comprising a modified beta cell to the subject, and wherein the total daily dosage of sirolimus administered to the subject yields a blood trough level of between about 7 ng/mL and about 10 ng/mL, inclusive of each, after the first three months. In some embodiments, the total daily dosage of tacrolimus administered to the subject yields a blood trough level of between about 3 ng/mL and about 6 ng/mL, inclusive of each. In some embodiments, the sirolimus regimen is administered at a lower dose. In some embodiments, the tacrolimus regimen is administered at a lower dose. In some embodiments, the daclizumab regimen is administered at a lower dose. In some embodiments, the subject is not administered glucocorticoids.

In some embodiments, the one or more immunosuppressive agents comprise alemtuzumab. In some embodiments, the alemtuzumab (e.g. a regimen of alemtuzumab) is administered to the subject prior to, concurrent with, and/or after the administration of the composition comprising a modified beta cell to the subject. In some embodiments, at least one regimen of alemtuzumab is administered to the subject prior to, concurrent with, and/or after the administration of the composition comprising a modified beta cell to the subject.

In some embodiments, the alemtuzumab (e.g. a regimen of alemtuzumab) is administered to the subject prior to the administration of the composition comprising a modified beta cell to the subject. In some embodiments, the alemtuzumab is administered to the subject only prior to the administration of the composition comprising a modified beta cell to the subject. In some embodiments, at least one regimen of alemtuzumab is administered to the subject prior to the administration of the composition comprising a modified beta cell to the subject. In some embodiments, more than one regimen of alemtuzumab is administered to the subject prior to administration of the composition comprising a modified beta cell to the subject. In some embodiments, the alemtuzumab (e.g. at least one regimen of alemtuzumab) is administered between about 30 seconds and about 10 weeks prior to administration of the composition comprising a modified beta cell to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks prior to administration of the composition comprising a modified beta cell to the subject. In some embodiments, the alemtuzumab is administered at least about 30 seconds prior to administration of the composition comprising a modified beta cell to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, or more, prior to administration of the composition comprising a modified beta cell to the subject. In some embodiments, the alemtuzumab is administered less than about 10 weeks prior to administration of the composition comprising a modified beta cell to the subject, such less than about any of 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the composition comprising a modified beta cell to the subject. In some embodiments, the alemtuzumab is administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the composition comprising a modified beta cell to the subject.

In some embodiments, the alemtuzumab (e.g. a regimen of alemtuzumab, such as a first regimen) is administered to the subject on the same day as the administration of the composition comprising a modified beta cell to the subject. In some embodiments, at least one regimen of alemtuzumab is administered to the subject on the same day as the administration of the composition comprising a modified beta cell to the subject. In some embodiments, the alemtuzumab is administered to the subject concurrent with administration of the composition comprising a modified beta cell to the subject. In some embodiments, at least one regimen of alemtuzumab is administered to the subject concurrent with administration of the composition comprising a modified beta cell to the subject. In some embodiments, the alemtuzumab is administered to the subject on the same day as administration of the composition comprising a modified beta cell to the subject, and continued to be administered over the course of the subject's lifespan. In some embodiments, the alemtuzumab is administered to the subject concurrent with administration of the composition comprising a modified beta cell to the subject, and continued to be administered over the course of the subject's lifespan. In some embodiments, a first regimen of the alemtuzumab is administered to the subject concurrent with administration of the composition comprising a modified beta cell to the subject. In some embodiments, a second regimen the alemtuzumab is administered to the subject concurrent with administration of the composition comprising a modified beta cell to the subject. In some embodiments, the alemtuzumab is administered to the subject on the same day as each round of administration of the composition comprising a modified beta cell. In some embodiments, the alemtuzumab is administered to the subject on the same day as each round of administration of the composition comprising a modified beta cell, and continued to be administered over the course of the subject's lifespan. In some embodiments, the alemtuzumab is administered to the subject concurrent with each round of administration of the composition comprising a modified beta cell. In some embodiments, the alemtuzumab is administered to the subject concurrent with each round of administration of the composition comprising a modified beta cell, and continued to be administered over the course of the subject's lifespan.

In some embodiments, the alemtuzumab (e.g. a regimen of alemtuzumab) is administered to the subject after administration of the composition comprising a modified beta cell to the subject. In some embodiments, at least one regimen of alemtuzumab is administered to the subject after administration of the composition comprising a modified beta cell to the subject. In some embodiments, more than one regimen of alemtuzumab is administered to the subject after administration of the composition comprising a modified beta cell to the subject. In some embodiments, the alemtuzumab is administered to the subject only after administration of a first and/or second regimen of the composition comprising a modified beta cell to the subject. In some embodiments, the alemtuzumab is administered between about 30 seconds and about 60 months after administration of the composition comprising a modified beta cell to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks, or between about 10 weeks and about 60 months, after administration of the composition comprising a modified beta cell to the subject. In some embodiments, the alemtuzumab is administered at least about 30 seconds after administration of the composition comprising a modified beta cell to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 12 months, 24 months, 60 months, or more, after administration of the composition comprising a modified beta cell to the subject. In some embodiments, the alemtuzumab is administered less than about 60 months after administration of the composition comprising a modified beta cell to the subject, such less than about any of 24 months, 12 months, 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the composition comprising a modified beta cell to the subject. In some embodiments, the alemtuzumab is administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds after administration of the composition comprising a modified beta cell to the subject. In some embodiments, the alemtuzumab is administered to the subject after administration of the composition comprising a modified beta cell, and continued to be administered over the course of the subject's lifespan. In some embodiments, the alemtuzumab is administered to the subject after each round of administration of the composition comprising a modified beta cell. In some embodiments, the alemtuzumab is administered to the subject after each round of administration of the composition comprising a modified beta cell, and continued to be administered over the course of the subject's lifespan. In some embodiments, the alemtuzumab is administered to the subject each day after the composition comprising a modified beta cell is administered to the subject.

In some embodiments, the subject is administered a regimen of alemtuzumab and a regimen of tacrolimus. In some embodiments, the subject is administered at least one regimen of alemtuzumab and at least one regimen of tacrolimus. In some embodiments, the subject is administered the at least one regimen of alemtuzumab prior to, on the same day as, concurrent with, and/or after the at least one regimen of tacrolimus. In some embodiments, the subject is administered the at least one regimen of alemtuzumab prior to the at least one regimen of tacrolimus. In some embodiments, the subject is administered a regimen of alemtuzumab and a regimen of tacrolimus after the composition comprising a modified beta cell is administered to the subject. In some embodiments, the subject is administered a regimen of alemtuzumab and a regimen of MPA. In some embodiments, the subject is administered at least one regimen of alemtuzumab and at least one regimen of MPA. In some embodiments, the subject is administered the at least one regimen of alemtuzumab prior to, on the same day as, concurrent with, and/or after the at least one regimen of MPA. In some embodiments, the subject is administered the at least one regimen of alemtuzumab prior to the at least one regimen of MPA. In some embodiments, the subject is administered a regimen of alemtuzumab and a regimen of MPA after the composition comprising a modified beta cell is administered to the subject. In some embodiments, the subject is administered a regimen of alemtuzumab, a regimen of tacrolimus, and a regimen of MPA. In some embodiments, the subject is administered at least one regimen of alemtuzumab, at least one regimen of tacrolimus, and at least one regimen of MPA. In some embodiments, the subject is administered the at least one regimen of alemtuzumab prior to, on the same day as, concurrent with, and/or after the at least one regimen of tacrolimus and the at least one regimen of MPA. In some embodiments, the subject is administered the at least one regimen of alemtuzumab prior to the at least one regimen of tacrolimus and the at least one regimen of MPA. In some embodiments, the subject is administered a regimen of alemtuzumab, a regimen of tacrolimus, and a regimen of MPA after the composition comprising a modified beta cell is administered to the subject.

i. Antibiotic Agents

In some aspects, the one or more immunosuppressive agents comprise an antibiotic agent (e.g., one or more antibiotic agents). Antibiotic agents are a type of antimicrobial substance active against bacteria. In some embodiments, the antibiotic agent may be, but is not limited to, trimethoprim/sulfamethoxaxole, penicillin, amoxicillin, cephalexin, erythromycin (E-Mycin), clarithromycin (Biaxin), azithromycin (Zithromax), ciprofolxacin (Cipro), levofloxacin (Levaquin), ofloxacin (Floxin), co-trimoxazole (Bactrim) and trimethoprim (Proloprim), tetracycline (Sumycin, Panmycin) and doxycycline (Vibramycin), gentamicin (Garamycin), or tobramycin (Tobrex). In some embodiments, the antibiotic agent is trimethoprim/sulfamethoxaxole. In some embodiments, the antibiotic agent is administered to the subject (e.g. one or more regimens of the antibiotic agent is administered to the subject). In some embodiments, the antibiotic agent is administered to the subject in one or more compositions, e.g. a pharmaceutical composition containing the antibiotic agent. In some embodiments, the antibiotic agent is administered to the subject prior to, concurrent with, and/or after the administration of the composition comprising a modified beta cell to the subject. In some embodiments, at least one regimen of antibiotic agent is administered to the subject prior to, concurrent with, and/or after the administration of the composition comprising a modified beta cell to the subject.

In some embodiments, the one or more immunosuppressive agents comprise trimethoprim/sulfamethoxaxole. In some embodiments, the trimethoprim/sulfamethoxaxole (e.g. a regimen of trimethoprim/sulfamethoxaxole) is administered to the subject prior to, concurrent with, and/or after the administration of the composition comprising a modified beta cell to the subject. In some embodiments, at least one regimen of trimethoprim/sulfamethoxaxole is administered to the subject prior to, concurrent with, and/or after the administration of the composition comprising a modified beta cell to the subject.

In some embodiments, the trimethoprim/sulfamethoxaxole (e.g. a regimen of trimethoprim/sulfamethoxaxole) is administered to the subject prior to the administration of the composition comprising a modified beta cell to the subject. In some embodiments, the trimethoprim/sulfamethoxaxole is administered to the subject only prior to the administration of the composition comprising a modified beta cell to the subject. In some embodiments, at least one regimen of trimethoprim/sulfamethoxaxole is administered to the subject prior to the administration of the composition comprising a modified beta cell to the subject. In some embodiments, more than one regimen of trimethoprim/sulfamethoxaxole is administered to the subject prior to administration of the composition comprising a modified beta cell to the subject. In some embodiments, the trimethoprim/sulfamethoxaxole (e.g. at least one regimen of trimethoprim/sulfamethoxaxole) is administered between about 30 seconds and about 10 weeks prior to administration of the composition comprising a modified beta cell to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks prior to administration of the composition comprising a modified beta cell to the subject. In some embodiments, the trimethoprim/sulfamethoxaxole is administered at least about 30 seconds prior to administration of the composition comprising a modified beta cell to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, or more, prior to administration of the composition comprising a modified beta cell to the subject. In some embodiments, the trimethoprim/sulfamethoxaxole is administered less than about 10 weeks prior to administration of the composition comprising a modified beta cell to the subject, such less than about any of 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the composition comprising a modified beta cell to the subject. In some embodiments, the trimethoprim/sulfamethoxaxole is administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the composition comprising a modified beta cell to the subject.

In some embodiments, the trimethoprim/sulfamethoxaxole (e.g. a regimen of trimethoprim/sulfamethoxaxole, such as a first regimen) is administered to the subject on the same day as the administration of the composition comprising a modified beta cell to the subject. In some embodiments, at least one regimen of trimethoprim/sulfamethoxaxole is administered to the subject on the same day as the administration of the composition comprising a modified beta cell to the subject. In some embodiments, the trimethoprim/sulfamethoxaxole is administered to the subject concurrent with administration of the composition comprising a modified beta cell to the subject. In some embodiments, at least one regimen of trimethoprim/sulfamethoxaxole is administered to the subject concurrent with administration of the composition comprising a modified beta cell to the subject. In some embodiments, the trimethoprim/sulfamethoxaxole is administered to the subject on the same day as administration of the composition comprising a modified beta cell to the subject, and continued to be administered over the course of the subject's lifespan. In some embodiments, the trimethoprim/sulfamethoxaxole is administered to the subject concurrent with administration of the composition comprising a modified beta cell to the subject, and continued to be administered over the course of the subject's lifespan. In some embodiments, a first regimen of the trimethoprim/sulfamethoxaxole is administered to the subject concurrent with administration of the composition comprising a modified beta cell to the subject. In some embodiments, a second regimen the trimethoprim/sulfamethoxaxole is administered to the subject concurrent with administration of the composition comprising a modified beta cell to the subject. In some embodiments, the trimethoprim/sulfamethoxaxole is administered to the subject on the same day as each round of administration of the composition comprising a modified beta cell. In some embodiments, the trimethoprim/sulfamethoxaxole is administered to the subject on the same day as each round of administration of the composition comprising a modified beta cell, and continued to be administered over the course of the subject's lifespan. In some embodiments, the trimethoprim/sulfamethoxaxole is administered to the subject concurrent with each round of administration of the composition comprising a modified beta cell. In some embodiments, the trimethoprim/sulfamethoxaxole is administered to the subject concurrent with each round of administration of the composition comprising a modified beta cell, and continued to be administered over the course of the subject's lifespan.

In some embodiments, the trimethoprim/sulfamethoxaxole (e.g. a regimen of trimethoprim/sulfamethoxaxole) is administered to the subject after administration of the composition comprising a modified beta cell to the subject. In some embodiments, at least one regimen of trimethoprim/sulfamethoxaxole is administered to the subject after administration of the composition comprising a modified beta cell to the subject. In some embodiments, more than one regimen of trimethoprim/sulfamethoxaxole is administered to the subject after administration of the composition comprising a modified beta cell to the subject. In some embodiments, the trimethoprim/sulfamethoxaxole is administered to the subject only after administration of a first and/or second regimen of the composition comprising a modified beta cell to the subject. In some embodiments, the trimethoprim/sulfamethoxaxole is administered between about 30 seconds and about 60 months after administration of the composition comprising a modified beta cell to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks, or between about 10 weeks and about 60 months, after administration of the composition comprising a modified beta cell to the subject. In some embodiments, the trimethoprim/sulfamethoxaxole is administered at least about 30 seconds after administration of the composition comprising a modified beta cell to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 12 months, 24 months, 60 months, or more, after administration of the composition comprising a modified beta cell to the subject. In some embodiments, the trimethoprim/sulfamethoxaxole is administered less than about 60 months after administration of the composition comprising a modified beta cell to the subject, such less than about any of 24 months, 12 months, 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the composition comprising a modified beta cell to the subject. In some embodiments, the trimethoprim/sulfamethoxaxole is administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds after administration of the composition comprising a modified beta cell to the subject. In some embodiments, a regimen of trimethoprim/sulfamethoxaxole is administered to the subject about 6 months after the administration of the composition comprising a modified beta cell to the subject. In some embodiments, the trimethoprim/sulfamethoxaxole is administered to the subject after administration of the composition comprising a modified beta cell, and continued to be administered over the course of the subject's lifespan. In some embodiments, the trimethoprim/sulfamethoxaxole is administered to the subject after each round of administration of the composition comprising a modified beta cell. In some embodiments, the trimethoprim/sulfamethoxaxole is administered to the subject after each round of administration of the composition comprising a modified beta cell, and continued to be administered over the course of the subject's lifespan. In some embodiments, the trimethoprim/sulfamethoxaxole is administered to the subject each day after the composition comprising a modified beta cell is administered to the subject.

In some embodiments, a regimen or a total daily dose of between about 10 mg and about 1,000 mg of trimethoprim/sulfamethoxaxole is administered to the subject, such as between about 10 mg and about 50 mg, between about 25 mg and about 100 mg, or between about 50 mg and about 1,000 mg. In some embodiments, the total daily dose of trimethoprim/sulfamethoxaxole administered to the subject is between about 80 mg and about 400 mg. In some embodiments, the total daily dose of trimethoprim/sulfamethoxaxole administered to the subject is greater than about 10 mg, such as greater than any of about 20 mg, 30 mg, 40 mg, 50 mg, 100 mg, 200 mg, 300 mg, 400 mg, 500 mg, 1,000 mg, or greater, of trimethoprim/sulfamethoxaxole. In some embodiments, the total daily dose of trimethoprim/sulfamethoxaxole administered to the subject is less than about 1,000 mg, such as less than any of about 500 mg, 400 mg, 300 mg, 200 mg, 100 mg, 50 mg, 40 mg, 30 mg, 20 mg, 10 mg, or less, of trimethoprim/sulfamethoxaxole.

j. Antifungal Agents

In some aspects, the one or more immunosuppressive agents comprise an antifungal agent (e.g., one or more antifungal agents). Antifungal agents are a type of pharmaceutical fungicide or fungistatic used to treat and prevent mycosis such as athlete's foot, ringworm, candidiasis, serious systemic infections such as cryptococcal meningitis, and others. In some embodiments, the antifungal agent may be, but is not limited to, clotrimazole, miconazole, ketoconazole, itraconazole, or fluconazole. In some embodiments, the antifungal agent is clotrimazole. In some embodiments, the antifungal agent is administered to the subject (e.g. one or more regimens of the antifungal agent is administered to the subject). In some embodiments, the antifungal agent is administered to the subject in one or more compositions, e.g. a pharmaceutical composition containing the antifungal agent. In some embodiments, the antifungal agent is administered to the subject prior to, concurrent with, and/or after the administration of the composition comprising a modified beta cell to the subject. In some embodiments, at least one regimen of antifungal agent is administered to the subject prior to, concurrent with, and/or after the administration of the composition comprising a modified beta cell to the subject.

In some embodiments, the one or more immunosuppressive agents comprise clotrimazole. In some embodiments, the clotrimazole (e.g. a regimen of clotrimazole) is administered to the subject prior to, concurrent with, and/or after the administration of the composition comprising a modified beta cell to the subject. In some embodiments, at least one regimen of clotrimazole is administered to the subject prior to, concurrent with, and/or after the administration of the composition comprising a modified beta cell to the subject.

In some embodiments, the clotrimazole (e.g. a regimen of clotrimazole) is administered to the subject prior to the administration of the composition comprising a modified beta cell to the subject. In some embodiments, the clotrimazole is administered to the subject only prior to the administration of the composition comprising a modified beta cell to the subject. In some embodiments, at least one regimen of clotrimazole is administered to the subject prior to the administration of the composition comprising a modified beta cell to the subject. In some embodiments, more than one regimen of clotrimazole is administered to the subject prior to administration of the composition comprising a modified beta cell to the subject. In some embodiments, the clotrimazole (e.g. at least one regimen of clotrimazole) is administered between about 30 seconds and about 10 weeks prior to administration of the composition comprising a modified beta cell to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks prior to administration of the composition comprising a modified beta cell to the subject. In some embodiments, the clotrimazole is administered at least about 30 seconds prior to administration of the composition comprising a modified beta cell to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, or more, prior to administration of the composition comprising a modified beta cell to the subject. In some embodiments, the clotrimazole is administered less than about 10 weeks prior to administration of the composition comprising a modified beta cell to the subject, such less than about any of 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the composition comprising a modified beta cell to the subject. In some embodiments, the clotrimazole is administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the composition comprising a modified beta cell to the subject. In some embodiments, the clotrimazole is administered to the subject about four times each day prior to the composition comprising a modified beta cell is administered to the subject.

In some embodiments, the clotrimazole (e.g. a regimen of clotrimazole, such as a first regimen) is administered to the subject on the same day as the administration of the composition comprising a modified beta cell to the subject. In some embodiments, at least one regimen of clotrimazole is administered to the subject on the same day as the administration of the composition comprising a modified beta cell to the subject. In some embodiments, the clotrimazole is administered to the subject concurrent with administration of the composition comprising a modified beta cell to the subject. In some embodiments, at least one regimen of clotrimazole is administered to the subject concurrent with administration of the composition comprising a modified beta cell to the subject. In some embodiments, the clotrimazole is administered to the subject on the same day as administration of the composition comprising a modified beta cell to the subject, and continued to be administered over the course of the subject's lifespan. In some embodiments, the clotrimazole is administered to the subject concurrent with administration of the composition comprising a modified beta cell to the subject, and continued to be administered over the course of the subject's lifespan. In some embodiments, a first regimen of the clotrimazole is administered to the subject concurrent with administration of the composition comprising a modified beta cell to the subject. In some embodiments, a second regimen the clotrimazole is administered to the subject concurrent with administration of the composition comprising a modified beta cell to the subject. In some embodiments, the clotrimazole is administered to the subject on the same day as each round of administration of the composition comprising a modified beta cell. In some embodiments, the clotrimazole is administered to the subject on the same day as each round of administration of the composition comprising a modified beta cell, and continued to be administered over the course of the subject's lifespan. In some embodiments, the clotrimazole is administered to the subject concurrent with each round of administration of the composition comprising a modified beta cell. In some embodiments, the clotrimazole is administered to the subject concurrent with each round of administration of the composition comprising a modified beta cell, and continued to be administered over the course of the subject's lifespan.

In some embodiments, the clotrimazole (e.g. a regimen of clotrimazole) is administered to the subject after administration of the composition comprising a modified beta cell to the subject. In some embodiments, at least one regimen of clotrimazole is administered to the subject after administration of the composition comprising a modified beta cell to the subject. In some embodiments, more than one regimen of clotrimazole is administered to the subject after administration of the composition comprising a modified beta cell to the subject. In some embodiments, the clotrimazole is administered to the subject only after administration of a first and/or second regimen of the composition comprising a modified beta cell to the subject. In some embodiments, the clotrimazole is administered between about 30 seconds and about 60 months after administration of the composition comprising a modified beta cell to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks, or between about 10 weeks and about 60 months, after administration of the composition comprising a modified beta cell to the subject. In some embodiments, the clotrimazole is administered at least about 30 seconds after administration of the composition comprising a modified beta cell to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 12 months, 24 months, 60 months, or more, after administration of the composition comprising a modified beta cell to the subject. In some embodiments, the clotrimazole is administered less than about 60 months after administration of the composition comprising a modified beta cell to the subject, such less than about any of 24 months, 12 months, 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the composition comprising a modified beta cell to the subject. In some embodiments, the clotrimazole is administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds after administration of the composition comprising a modified beta cell to the subject. In some embodiments, the clotrimazole is administered to the subject after administration of the composition comprising a modified beta cell, and continued to be administered over the course of the subject's lifespan. In some embodiments, the clotrimazole is administered to the subject after each round of administration of the composition comprising a modified beta cell. In some embodiments, the clotrimazole is administered to the subject after each round of administration of the composition comprising a modified beta cell, and continued to be administered over the course of the subject's lifespan. In some embodiments, the clotrimazole is administered to the subject each day after the composition comprising a modified beta cell is administered to the subject. In some embodiments, the clotrimazole is administered to the subject each day for up to about 3 months after the composition comprising a modified beta cell is administered to the subject. In some embodiments, the clotrimazole is administered to the subject about four times each day after the composition comprising a modified beta cell is administered to the subject. In some embodiments, the clotrimazole is administered to the subject about four times each day after the composition comprising a modified beta cell is administered to the subject.

k. Antiviral Agents

In some aspects, the one or more immunosuppressive agents comprise an antiviral agent (e.g., one or more antiviral agents). Antiviral agents are used for the treatment or control of viral infections, and generally target stages in the viral life cycle. In some embodiments, the antiviral agent may be, but is not limited to, darunavir, atazanavir, ritonavir, acyclovir, valacyclovir, valganciclovir, tenofovir, and raltegravir. In some embodiments, the antiviral agent is an anti-cytomegaloviral agent. In some embodiments, the antiviral agent is valacyclovir. In some embodiments, the antiviral agent is administered to the subject (e.g. one or more regimens of the antiviral agent is administered to the subject). In some embodiments, the antiviral agent is administered to the subject in one or more compositions, e.g. a pharmaceutical composition containing the antiviral agent. In some embodiments, the antiviral agent is administered to the subject prior to, concurrent with, and/or after the administration of the composition comprising a modified beta cell to the subject. In some embodiments, at least one regimen of antiviral agent is administered to the subject prior to, concurrent with, and/or after the administration of the composition comprising a modified beta cell to the subject.

In some embodiments, the one or more immunosuppressive agents comprise valacyclovir. In some embodiments, the valacyclovir (e.g. a regimen of valacyclovir) is administered to the subject prior to, concurrent with, and/or after the administration of the composition comprising a modified beta cell to the subject. In some embodiments, at least one regimen of valacyclovir is administered to the subject prior to, concurrent with, and/or after the administration of the composition comprising a modified beta cell to the subject.

In some embodiments, the valacyclovir (e.g. a regimen of valacyclovir) is administered to the subject prior to the administration of the composition comprising a modified beta cell to the subject. In some embodiments, the valacyclovir is administered to the subject only prior to the administration of the composition comprising a modified beta cell to the subject. In some embodiments, at least one regimen of valacyclovir is administered to the subject prior to the administration of the composition comprising a modified beta cell to the subject. In some embodiments, more than one regimen of valacyclovir is administered to the subject prior to administration of the composition comprising a modified beta cell to the subject. In some embodiments, the valacyclovir (e.g. at least one regimen of valacyclovir) is administered between about 30 seconds and about 10 weeks prior to administration of the composition comprising a modified beta cell to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks prior to administration of the composition comprising a modified beta cell to the subject. In some embodiments, the valacyclovir is administered at least about 30 seconds prior to administration of the composition comprising a modified beta cell to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, or more, prior to administration of the composition comprising a modified beta cell to the subject. In some embodiments, the clotrimazole is administered less than about 10 weeks prior to administration of the composition comprising a modified beta cell to the subject, such less than about any of 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the composition comprising a modified beta cell to the subject. In some embodiments, the valacyclovir is administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the composition comprising a modified beta cell to the subject.

In some embodiments, the valacyclovir (e.g. a regimen of valacyclovir, such as a first regimen) is administered to the subject on the same day as the administration of the composition comprising a modified beta cell to the subject. In some embodiments, at least one regimen of valacyclovir is administered to the subject on the same day as the administration of the composition comprising a modified beta cell to the subject. In some embodiments, the valacyclovir is administered to the subject concurrent with administration of the composition comprising a modified beta cell to the subject. In some embodiments, at least one regimen of valacyclovir is administered to the subject concurrent with administration of the composition comprising a modified beta cell to the subject. In some embodiments, the valacyclovir is administered to the subject on the same day as administration of the composition comprising a modified beta cell to the subject, and continued to be administered over the course of the subject's lifespan. In some embodiments, the valacyclovir is administered to the subject concurrent with administration of the composition comprising a modified beta cell to the subject, and continued to be administered over the course of the subject's lifespan. In some embodiments, a first regimen of the valacyclovir is administered to the subject concurrent with administration of the composition comprising a modified beta cell to the subject. In some embodiments, a second regimen the valacyclovir is administered to the subject concurrent with administration of the composition comprising a modified beta cell to the subject. In some embodiments, the valacyclovir is administered to the subject on the same day as each round of administration of the composition comprising a modified beta cell. In some embodiments, the valacyclovir is administered to the subject on the same day as each round of administration of the composition comprising a modified beta cell, and continued to be administered over the course of the subject's lifespan. In some embodiments, the valacyclovir is administered to the subject concurrent with each round of administration of the composition comprising a modified beta cell. In some embodiments, the valacyclovir is administered to the subject concurrent with each round of administration of the composition comprising a modified beta cell, and continued to be administered over the course of the subject's lifespan.

In some embodiments, the valacyclovir (e.g. a regimen of valacyclovir) is administered to the subject after administration of the composition comprising a modified beta cell to the subject. In some embodiments, at least one regimen of valacyclovir is administered to the subject after administration of the composition comprising a modified beta cell to the subject. In some embodiments, more than one regimen of valacyclovir is administered to the subject after administration of the composition comprising a modified beta cell to the subject. In some embodiments, the valacyclovir is administered to the subject only after administration of a first and/or second regimen of the composition comprising a modified beta cell to the subject. In some embodiments, the valacyclovir is administered between about 30 seconds and about 60 months after administration of the composition comprising a modified beta cell to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks, or between about 10 weeks and about 60 months, after administration of the composition comprising a modified beta cell to the subject. In some embodiments, the valacyclovir is administered at least about 30 seconds after administration of the composition comprising a modified beta cell to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 12 months, 24 months, 60 months, or more, after administration of the composition comprising a modified beta cell to the subject. In some embodiments, the valacyclovir is administered less than about 60 months after administration of the composition comprising a modified beta cell to the subject, such less than about any of 24 months, 12 months, 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the composition comprising a modified beta cell to the subject. In some embodiments, the valacyclovir is administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds after administration of the composition comprising a modified beta cell to the subject. In some embodiments, the valacyclovir is administered to the subject after administration of the composition comprising a modified beta cell, and continued to be administered over the course of the subject's lifespan. In some embodiments, the valacyclovir is administered to the subject after each round of administration of the composition comprising a modified beta cell. In some embodiments, the valacyclovir is administered to the subject after each round of administration of the composition comprising a modified beta cell, and continued to be administered over the course of the subject's lifespan. In some embodiments, the valacyclovir is administered to the subject each day after the composition comprising a modified beta cell is administered to the subject. In some embodiments, the valacyclovir is administered to the subject each day after about 12 days after the composition comprising a modified beta cell is administered to the subject. In some embodiments, the valacyclovir is administered to the subject for about 14 weeks after the composition comprising a modified beta cell is administered to the subject. In some embodiments, the valacyclovir is administered to the subject each day after about 12 days after the composition comprising a modified beta cell is administered to the subject, and continued for about 14 weeks after the composition comprising a modified beta cell is administered to the subject.

In some embodiments, a regimen and/or total daily dose of between about 200 mg and about 2,000 mg valacyclovir is administered to the subject, such as a regimen of between about 200 mg and about 500 mg valacyclovir, between about 400 mg and about 800 mg, or between about 600 mg and about 2,000 mg. In some embodiments, a regimen of between about 300 mg and about 1,000 mg of valacyclovir is administered to the subject. In some embodiments, a regimen of greater than about 200 mg of valacyclovir is administered to the subject, such as a regimen of greater than any of about 300 mg, 400 mg, 500 mg, 600 mg, 700 mg, 800 mg, 900 mg, 1,000 mg, 1,500 mg, 2,000 mg, or greater, of valacyclovir. In some embodiments, a regimen of less than about 2,000 mg of valacyclovir is administered to the subject, such as a regimen of less than any of about 500 mg, 400 mg, 300 mg, 200 mg, 100 mg, 50 mg, 10 mg, 5 mg, 4 mg, 3 mg, 2 mg, 1 mg, or less, of valacyclovir. In some embodiments, a regimen of about 450 mg valacyclovir is administered to the subject. In some embodiments, a regimen of about 700 mg valacyclovir is administered to the subject.

In some embodiments, a regimen of about 450 mg valacyclovir is administered to the subject after the administration of the composition comprising a modified beta cell to the subject. In some embodiments, a regimen of about 450 mg valacyclovir is administered to the subject each day after the administration of the composition comprising a modified beta cell to the subject. In some embodiments, a regimen of about 450 mg valacyclovir is administered to the subject each day after the administration of the composition comprising a modified beta cell to the subject, up to about 12 days after the administration of the composition comprising a modified beta cell to the subject. In some embodiments, a regimen of about 900 mg valacyclovir is administered to the subject after the administration of the composition comprising a modified beta cell to the subject. In some embodiments, a regimen of about 900 mg valacyclovir is administered to the subject each day after the administration of the composition comprising a modified beta cell to the subject. In some embodiments, a regimen of about 900 mg valacyclovir is administered to the subject each day after the administration of the composition comprising a modified beta cell to the subject after about 12 days after the administration of the composition comprising a modified beta cell to the subject. In some embodiments, the valacyclovir regimen is administered at a lower dose.

l. Hemorheologic Agents

In some aspects, the one or more immunosuppressive agents comprise a hemorheologic agent (e.g., one or more hemorheologic agents). Hemorheologic agents are drugs that increase the blood flow in arteries. In some embodiments, the hemorheologic agent is pentoxifylline. In some embodiments, the hemorheologic agent is administered to the subject (e.g. one or more regimens of the hemorheologic agent is administered to the subject). In some embodiments, the hemorheologic agent is administered to the subject in one or more compositions, e.g. a pharmaceutical composition containing the hemorheologic agent. In some embodiments, the hemorheologic agent is administered to the subject prior to, concurrent with, and/or after the administration of the composition comprising a modified beta cell to the subject. In some embodiments, at least one regimen of hemorheologic agent is administered to the subject prior to, concurrent with, and/or after the administration of the composition comprising a modified beta cell to the subject.

In some embodiments, the one or more immunosuppressive agents comprise pentoxifylline. In some embodiments, the pentoxifylline (e.g. a regimen of pentoxifylline) is administered to the subject prior to, concurrent with, and/or after the administration of the composition comprising a modified beta cell to the subject. In some embodiments, at least one regimen of pentoxifylline is administered to the subject prior to, concurrent with, and/or after the administration of the composition comprising a modified beta cell to the subject.

In some embodiments, the pentoxifylline (e.g. a regimen of pentoxifylline) is administered to the subject prior to the administration of the composition comprising a modified beta cell to the subject. In some embodiments, the pentoxifylline is administered to the subject only prior to the administration of the composition comprising a modified beta cell to the subject. In some embodiments, at least one regimen of pentoxifylline is administered to the subject prior to the administration of the composition comprising a modified beta cell to the subject. In some embodiments, more than one regimen of pentoxifylline is administered to the subject prior to administration of the composition comprising a modified beta cell to the subject. In some embodiments, the pentoxifylline (e.g. at least one regimen of pentoxifylline) is administered between about 30 seconds and about 10 weeks prior to administration of the composition comprising a modified beta cell to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks prior to administration of the composition comprising a modified beta cell to the subject. In some embodiments, the pentoxifylline is administered at least about 30 seconds prior to administration of the composition comprising a modified beta cell to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, or more, prior to administration of the composition comprising a modified beta cell to the subject. In some embodiments, the clotrimazole is administered less than about 10 weeks prior to administration of the composition comprising a modified beta cell to the subject, such less than about any of 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the composition comprising a modified beta cell to the subject. In some embodiments, the pentoxifylline is administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the composition comprising a modified beta cell to the subject. In some embodiments, the pentoxifylline is administered about 2 days prior to administration of the composition comprising a modified beta cell to the subject.

In some embodiments, the pentoxifylline (e.g. a regimen of pentoxifylline, such as a first regimen) is administered to the subject on the same day as the administration of the composition comprising a modified beta cell to the subject. In some embodiments, at least one regimen of pentoxifylline is administered to the subject on the same day as the administration of the composition comprising a modified beta cell to the subject. In some embodiments, the pentoxifylline is administered to the subject concurrent with administration of the composition comprising a modified beta cell to the subject. In some embodiments, at least one regimen of pentoxifylline is administered to the subject concurrent with administration of the composition comprising a modified beta cell to the subject. In some embodiments, the pentoxifylline is administered to the subject on the same day as administration of the composition comprising a modified beta cell to the subject, and continued to be administered over the course of the subject's lifespan. In some embodiments, the pentoxifylline is administered to the subject concurrent with administration of the composition comprising a modified beta cell to the subject, and continued to be administered over the course of the subject's lifespan. In some embodiments, a first regimen of the pentoxifylline is administered to the subject concurrent with administration of the composition comprising a modified beta cell to the subject. In some embodiments, a second regimen the pentoxifylline is administered to the subject concurrent with administration of the composition comprising a modified beta cell to the subject. In some embodiments, the pentoxifylline is administered to the subject on the same day as each round of administration of the composition comprising a modified beta cell. In some embodiments, the pentoxifylline is administered to the subject on the same day as each round of administration of the composition comprising a modified beta cell, and continued to be administered over the course of the subject's lifespan. In some embodiments, the pentoxifylline is administered to the subject concurrent with each round of administration of the composition comprising a modified beta cell. In some embodiments, the pentoxifylline is administered to the subject concurrent with each round of administration of the composition comprising a modified beta cell, and continued to be administered over the course of the subject's lifespan.

In some embodiments, the pentoxifylline (e.g. a regimen of pentoxifylline) is administered to the subject after administration of the composition comprising a modified beta cell to the subject. In some embodiments, at least one regimen of pentoxifylline is administered to the subject after administration of the composition comprising a modified beta cell to the subject. In some embodiments, more than one regimen of pentoxifylline is administered to the subject after administration of the composition comprising a modified beta cell to the subject. In some embodiments, the pentoxifylline is administered to the subject only after administration of a first and/or second regimen of the composition comprising a modified beta cell to the subject. In some embodiments, the pentoxifylline is administered between about 30 seconds and about 60 months after administration of the composition comprising a modified beta cell to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks, or between about 10 weeks and about 60 months, after administration of the composition comprising a modified beta cell to the subject. In some embodiments, the pentoxifylline is administered at least about 30 seconds after administration of the composition comprising a modified beta cell to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 12 months, 24 months, 60 months, or more, after administration of the composition comprising a modified beta cell to the subject. In some embodiments, the pentoxifylline is administered less than about 60 months after administration of the composition comprising a modified beta cell to the subject, such less than about any of 24 months, 12 months, 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the composition comprising a modified beta cell to the subject. In some embodiments, the pentoxifylline is administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds after administration of the composition comprising a modified beta cell to the subject. In some embodiments, the pentoxifylline is administered to the subject after administration of the composition comprising a modified beta cell, and continued to be administered over the course of the subject's lifespan. In some embodiments, the pentoxifylline is administered to the subject after each round of administration of the composition comprising a modified beta cell. In some embodiments, the pentoxifylline is administered to the subject after each round of administration of the composition comprising a modified beta cell, and continued to be administered over the course of the subject's lifespan. In some embodiments, the pentoxifylline is administered to the subject each day after the composition comprising a modified beta cell is administered to the subject. In some embodiments, the pentoxifylline is administered to the subject through about 7 days after the composition comprising a modified beta cell is administered to the subject.

In some embodiments, a regimen and/or total daily dose of between about 200 mg and about 1,000 mg pentoxifylline is administered to the subject, such as a regimen of between about 200 mg and about 500 mg pentoxifylline, between about 400 mg and about 800 mg, or between about 600 mg and about 1,000 mg. In some embodiments, a regimen of between about 300 mg and about 1,000 mg of pentoxifylline is administered to the subject. In some embodiments, a regimen of greater than about 200 mg of pentoxifylline is administered to the subject, such as a regimen of greater than any of about 300 mg, 400 mg, 500 mg, 600 mg, 700 mg, 800 mg, 900 mg, 1,000 mg, or greater, of pentoxifylline. In some embodiments, a regimen of less than about 1,000 mg of pentoxifylline is administered to the subject, such as a regimen of less than any of about 900 mg, 800 mg, 700 mg, 600 mg, 500 mg, 400 mg, 300 mg, 200 mg, or less, of pentoxifylline.

m. Anticoagulation Agents

In some aspects, the one or more immunosuppressive agents comprise an anticoagulation agent (e.g., one or more anticoagulation agents). Anticoagulation agents are drugs that help prevent blood clots. In some embodiments, the anticoagulation agent may be, but is not limited to, apixaban, dabigatran, edoxaban, rivaroxaban, warfarin, aspirin, enoxaparin, or heparin. In some embodiments, the anticoagulation agent is aspirin. In some embodiments, the anticoagulation agent is enoxaparin. In some embodiments, the anticoagulation agent is heparin. In some embodiments, the anticoagulation agent is administered to the subject (e.g. one or more regimens of the anticoagulation agent is administered to the subject). In some embodiments, the anticoagulation agent is administered to the subject in one or more compositions, e.g. a pharmaceutical composition containing the anticoagulation agent. In some embodiments, the anticoagulation agent is administered to the subject prior to, concurrent with, and/or after the administration of the composition comprising a modified beta cell to the subject. In some embodiments, at least one regimen of anticoagulation agent is administered to the subject prior to, concurrent with, and/or after the administration of the composition comprising a modified beta cell to the subject.

In some embodiments, the one or more immunosuppressive agents comprise aspirin. In some embodiments, the aspirin (e.g. a regimen of aspirin) is administered to the subject prior to, concurrent with, and/or after the administration of the composition comprising a modified beta cell to the subject. In some embodiments, at least one regimen of aspirin is administered to the subject prior to, concurrent with, and/or after the administration of the composition comprising a modified beta cell to the subject. In some embodiments, at least one regimen of aspirin is administered to the subject after the administration of the composition comprising a modified beta cell to the subject.

In some embodiments, the one or more immunosuppressive agents comprise enoxaparin. In some embodiments, the enoxaparin (e.g. a regimen of enoxaparin) is administered to the subject prior to, concurrent with, and/or after the administration of the composition comprising a modified beta cell to the subject. In some embodiments, at least one regimen of enoxaparin is administered to the subject prior to, concurrent with, and/or after the administration of the composition comprising a modified beta cell to the subject. In some embodiments, at least one regimen of enoxaparin is administered to the subject after the administration of the composition comprising a modified beta cell to the subject.

In some embodiments, the one or more immunosuppressive agents comprise heparin. In some embodiments, the heparin (e.g. a regimen of heparin) is administered to the subject prior to, concurrent with, and/or after the administration of the composition comprising a modified beta cell to the subject. In some embodiments, at least one regimen of heparin is administered to the subject prior to, concurrent with, and/or after the administration of the composition comprising a modified beta cell to the subject. In some embodiments, at least one regimen of heparin is administered to the subject after the administration of the composition comprising a modified beta cell to the subject.

n. DNA Synthesis Inhibitors

In some aspects, the one or more immunosuppressive agents comprise a DNA synthesis inhibitor (e.g., one or more DNA synthesis inhibitors). DNA synthesis inhibitors (e.g. nucleic acid synthesis inhibitors) can be used to prevent the synthesis of DNA, such as in the context of cancer. The DNA synthesis inhibitor may be an antimetabolite. In some embodiments, the DNA synthesis inhibitor is a purine analog. In some embodiments, the DNA synthesis inhibitor may be, but is not limited to, 5-fluorouracil (5-FU), capecitabine, fludarabine, floxuridine, cytarabine, gemcitabine, decitabine, or vidaza. In some embodiments, the DNA synthesis inhibitor is fludarabine. In some embodiments, the DNA synthesis inhibitor is administered to the subject (e.g. one or more regimens of the DNA synthesis inhibitor is administered to the subject). In some embodiments, the DNA synthesis inhibitor is administered to the subject in one or more compositions, e.g. a pharmaceutical composition containing the antiviral agent. In some embodiments, the DNA synthesis inhibitor is administered to the subject prior to, concurrent with, and/or after the administration of the composition comprising a modified beta cell to the subject. In some embodiments, at least one regimen of DNA synthesis inhibitor is administered to the subject prior to, concurrent with, and/or after the administration of the composition comprising a modified beta cell to the subject.

In some embodiments, the one or more immunosuppressive agents comprise fludarabine. In some embodiments, the fludarabine (e.g. a regimen of fludarabine) is administered to the subject prior to, concurrent with, and/or after the administration of the composition comprising a modified beta cell to the subject. In some embodiments, at least one regimen of fludarabine is administered to the subject prior to, concurrent with, and/or after the administration of the composition comprising a modified beta cell to the subject.

In some embodiments, the fludarabine (e.g. a regimen of fludarabine) is administered to the subject prior to the administration of the composition comprising a modified beta cell to the subject. In some embodiments, the fludarabine is administered to the subject only prior to the administration of the composition comprising a modified beta cell to the subject. In some embodiments, at least one regimen of fludarabine is administered to the subject prior to the administration of the composition comprising a modified beta cell to the subject. In some embodiments, more than one regimen of fludarabine is administered to the subject prior to administration of the composition comprising a modified beta cell to the subject. In some embodiments, the fludarabine (e.g. at least one regimen of fludarabine) is administered between about 30 seconds and about 10 weeks prior to administration of the composition comprising a modified beta cell to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks prior to administration of the composition comprising a modified beta cell to the subject. In some embodiments, the fludarabine is administered to the subject between about 2 days and about 14 days prior to the administration of the composition comprising a modified beta cell to the subject. In some embodiments, the fludarabine is administered at least about 30 seconds prior to administration of the composition comprising a modified beta cell to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, or more, prior to administration of the composition comprising a modified beta cell to the subject. In some embodiments, the fludarabine is administered less than about 10 weeks prior to administration of the composition comprising a modified beta cell to the subject, such less than about any of 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the composition comprising a modified beta cell to the subject. In some embodiments, the fludarabine is administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the composition comprising a modified beta cell to the subject. In some embodiments, the fludarabine is administered to the subject each day prior to the composition comprising a modified beta cell is administered to the subject. In some embodiments, the fludarabine is administered to the subject each day for about 2 days prior to the composition comprising a modified beta cell is administered to the subject. In some embodiments, the fludarabine is administered to the subject each day for about 3 days prior to the composition comprising a modified beta cell is administered to the subject. In some embodiments, the fludarabine is administered to the subject each day for about 4 days prior to the composition comprising a modified beta cell is administered to the subject. In some embodiments, the fludarabine is administered to the subject 5 days prior to the composition comprising a modified beta cell is administered to the subject. In some embodiments, the fludarabine is administered to the subject 4 days prior to the composition comprising a modified beta cell is administered to the subject. In some embodiments, the fludarabine is administered to the subject 3 days prior to the composition comprising a modified beta cell is administered to the subject. In some embodiments, the fludarabine is administered to the subject on day 5, day 4, and day 3, prior to the composition comprising a modified beta cell is administered to the subject.

In some embodiments, the fludarabine (e.g. a regimen of fludarabine, such as a first regimen) is administered to the subject on the same day as the administration of the composition comprising a modified beta cell to the subject. In some embodiments, at least one regimen of fludarabine is administered to the subject on the same day as the administration of the composition comprising a modified beta cell to the subject. In some embodiments, the fludarabine is administered to the subject concurrent with administration of the composition comprising a modified beta cell to the subject. In some embodiments, at least one regimen of fludarabine is administered to the subject concurrent with administration of the composition comprising a modified beta cell to the subject. In some embodiments, the fludarabine is administered to the subject on the same day as administration of the composition comprising a modified beta cell to the subject, and continued to be administered over the course of the subject's lifespan. In some embodiments, the fludarabine is administered to the subject concurrent with administration of the composition comprising a modified beta cell to the subject, and continued to be administered over the course of the subject's lifespan. In some embodiments, a first regimen of the fludarabine is administered to the subject concurrent with administration of the composition comprising a modified beta cell to the subject. In some embodiments, a second regimen the fludarabine is administered to the subject concurrent with administration of the composition comprising a modified beta cell to the subject. In some embodiments, the fludarabine is administered to the subject on the same day as each round of administration of the composition comprising a modified beta cell. In some embodiments, the fludarabine is administered to the subject on the same day as each round of administration of the composition comprising a modified beta cell, and continued to be administered over the course of the subject's lifespan. In some embodiments, the fludarabine is administered to the subject concurrent with each round of administration of the composition comprising a modified beta cell. In some embodiments, the fludarabine is administered to the subject concurrent with each round of administration of the composition comprising a modified beta cell, and continued to be administered over the course of the subject's lifespan.

In some embodiments, the fludarabine (e.g. a regimen of fludarabine) is administered to the subject after administration of the composition comprising a modified beta cell to the subject. In some embodiments, at least one regimen of fludarabine is administered to the subject after administration of the composition comprising a modified beta cell to the subject. In some embodiments, more than one regimen of fludarabine is administered to the subject after administration of the composition comprising a modified beta cell to the subject. In some embodiments, the fludarabine is administered to the subject only after administration of a first and/or second regimen of the composition comprising a modified beta cell to the subject. In some embodiments, the fludarabine is administered between about 30 seconds and about 60 months after administration of the composition comprising a modified beta cell to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks, or between about 10 weeks and about 60 months, after administration of the composition comprising a modified beta cell to the subject. In some embodiments, the fludarabine is administered at least about 30 seconds after administration of the composition comprising a modified beta cell to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 12 months, 24 months, 60 months, or more, after administration of the composition comprising a modified beta cell to the subject. In some embodiments, the fludarabine is administered less than about 60 months after administration of the composition comprising a modified beta cell to the subject, such less than about any of 24 months, 12 months, 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the composition comprising a modified beta cell to the subject. In some embodiments, the fludarabine is administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds after administration of the composition comprising a modified beta cell to the subject. In some embodiments, the fludarabine is administered to the subject after administration of the composition comprising a modified beta cell, and continued to be administered over the course of the subject's lifespan. In some embodiments, the fludarabine is administered to the subject after each round of administration of the composition comprising a modified beta cell. In some embodiments, the fludarabine is administered to the subject after each round of administration of the composition comprising a modified beta cell, and continued to be administered over the course of the subject's lifespan. In some embodiments, the fludarabine is administered to the subject each day after the composition comprising a modified beta cell is administered to the subject. In some embodiments, the fludarabine is administered to the subject each day for up to about 3 months after the composition comprising a modified beta cell is administered to the subject. In some embodiments, the fludarabine is administered to the subject about four times each day after the composition comprising a modified beta cell is administered to the subject. In some embodiments, the fludarabine is administered to the subject about four times each day after the composition comprising a modified beta cell is administered to the subject.

In some embodiments, a regimen and/or total daily dose of between about 5 mg/m² and about 100 mg/m² fludarabine is administered to the subject, such as a regimen of between about 5 mg/m² and about 40 mg/m², between about 20 mg/m² and about 80 mg/m², or between about 50 mg/m² and about 100 mg/m². In some embodiments, a regimen of between about 10 mg/m² and about 40 mg/m² of fludarabine is administered to the subject. In some embodiments, a regimen of greater than about 5 mg/m² of fludarabine is administered to the subject, such as a regimen of greater than any of about 10 mg/m², 20 mg/m², 30 mg/m², 40 mg/m², 50 mg/m², 60 mg/m², 70 mg/m², 80 mg/m², 90 mg/m², 100 mg/m², or greater, of fludarabine. In some embodiments, a regimen of less than about 100 mg/m² of fludarabine is administered to the subject, such as a regimen of less than any of about, 90 mg/m², 80 mg/m², 70 mg/m², 60 mg/m², 50 mg/m², 40 mg/m², 30 mg/m², 20 mg/m², 10 mg/m², 5 mg/m² or less, of fludarabine. In some embodiments, a regimen of about 30 mg/m² of fludarabine is administered to the subject. In some embodiments, fludarabine is administered to the subject intravenously.

o. Alkylating Agents

In some aspects, the one or more immunosuppressive agents comprise an alkylating agent (e.g., one or more alkylating agents). Alkylating agents may act by inhibiting the transcription of DNA into RNA, thereby stopping protein synthesis. Alkylating agents substitute alkyl groups for hydrogen atoms on DNA, resulting in the formation of cross links within the DNA chain and thereby resulting in cytotoxic, mutagenic, and carcinogenic effects. In some embodiments, the alkylating agent is a nitrogen mustard, an ethylenamine or methylenamine derivative, an alkyl sulfonate, a nitrosourea, a triazene, or a platinum-containing antineoplastic agent. In some embodiments, the alkylating agent may be, but is not limited to, mechlorethamine, cyclophosphamide, ifosfamide, melphalan, chlorambucil, altretamine, thiotepa, busulfan, carmustine, lomustine, dacarbazine, procarbazine, temozolomide, cisplatin, carboplatin, or oxaliplatin. In some embodiments, the alkylating agent is cyclophosphamide. In some embodiments, the alkylating agent is administered to the subject (e.g. one or more regimens of the alkylating agent is administered to the subject). In some embodiments, the alkylating agent is administered to the subject in one or more compositions, e.g. a pharmaceutical composition containing the antiviral agent. In some embodiments, the alkylating agent is administered to the subject prior to, concurrent with, and/or after the administration of the composition comprising a modified beta cell to the subject. In some embodiments, at least one regimen of alkylating agent is administered to the subject prior to, concurrent with, and/or after the administration of the composition comprising a modified beta cell to the subject.

In some embodiments, the one or more immunosuppressive agents comprise cyclophosphamide. In some embodiments, the cyclophosphamide (e.g. a regimen of cyclophosphamide) is administered to the subject prior to, concurrent with, and/or after the administration of the composition comprising a modified beta cell to the subject. In some embodiments, at least one regimen of cyclophosphamide is administered to the subject prior to, concurrent with, and/or after the administration of the composition comprising a modified beta cell to the subject.

In some embodiments, the cyclophosphamide (e.g. a regimen of cyclophosphamide) is administered to the subject prior to the administration of the composition comprising a modified beta cell to the subject. In some embodiments, the cyclophosphamide is administered to the subject only prior to the administration of the composition comprising a modified beta cell to the subject. In some embodiments, at least one regimen of cyclophosphamide is administered to the subject prior to the administration of the composition comprising a modified beta cell to the subject. In some embodiments, more than one regimen of cyclophosphamide is administered to the subject prior to administration of the composition comprising a modified beta cell to the subject. In some embodiments, the cyclophosphamide (e.g. at least one regimen of cyclophosphamide) is administered between about 30 seconds and about 10 weeks prior to administration of the composition comprising a modified beta cell to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks prior to administration of the composition comprising a modified beta cell to the subject. In some embodiments, the cyclophosphamide is administered to the subject between about 2 days and about 14 days prior to the administration of the composition comprising a modified beta cell to the subject. In some embodiments, the cyclophosphamide is administered at least about 30 seconds prior to administration of the composition comprising a modified beta cell to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, or more, prior to administration of the composition comprising a modified beta cell to the subject. In some embodiments, the cyclophosphamide is administered less than about 10 weeks prior to administration of the composition comprising a modified beta cell to the subject, such less than about any of 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the composition comprising a modified beta cell to the subject. In some embodiments, the cyclophosphamide is administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the composition comprising a modified beta cell to the subject. In some embodiments, the cyclophosphamide is administered to the subject each day prior to the composition comprising a modified beta cell is administered to the subject. In some embodiments, the cyclophosphamide is administered to the subject each day for about 2 days prior to the composition comprising a modified beta cell is administered to the subject. In some embodiments, the cyclophosphamide is administered to the subject each day for about 3 days prior to the composition comprising a modified beta cell is administered to the subject. In some embodiments, the cyclophosphamide is administered to the subject each day for about 4 days prior to the composition comprising a modified beta cell is administered to the subject. In some embodiments, the cyclophosphamide is administered to the subject 5 days prior to the composition comprising a modified beta cell is administered to the subject. In some embodiments, the cyclophosphamide is administered to the subject 4 days prior to the composition comprising a modified beta cell is administered to the subject. In some embodiments, the cyclophosphamide is administered to the subject 3 days prior to the composition comprising a modified beta cell is administered to the subject. In some embodiments, the cyclophosphamide is administered to the subject on day 5, day 4, and day 3, prior to the composition comprising a modified beta cell is administered to the subject.

In some embodiments, the cyclophosphamide (e.g. a regimen of cyclophosphamide, such as a first regimen) is administered to the subject on the same day as the administration of the composition comprising a modified beta cell to the subject. In some embodiments, at least one regimen of cyclophosphamide is administered to the subject on the same day as the administration of the composition comprising a modified beta cell to the subject. In some embodiments, the cyclophosphamide is administered to the subject concurrent with administration of the composition comprising a modified beta cell to the subject. In some embodiments, at least one regimen of cyclophosphamide is administered to the subject concurrent with administration of the composition comprising a modified beta cell to the subject. In some embodiments, the cyclophosphamide is administered to the subject on the same day as administration of the composition comprising a modified beta cell to the subject, and continued to be administered over the course of the subject's lifespan. In some embodiments, the cyclophosphamide is administered to the subject concurrent with administration of the composition comprising a modified beta cell to the subject, and continued to be administered over the course of the subject's lifespan. In some embodiments, a first regimen of the cyclophosphamide is administered to the subject concurrent with administration of the composition comprising a modified beta cell to the subject. In some embodiments, a second regimen the cyclophosphamide is administered to the subject concurrent with administration of the composition comprising a modified beta cell to the subject. In some embodiments, the cyclophosphamide is administered to the subject on the same day as each round of administration of the composition comprising a modified beta cell. In some embodiments, the cyclophosphamide is administered to the subject on the same day as each round of administration of the composition comprising a modified beta cell, and continued to be administered over the course of the subject's lifespan. In some embodiments, the cyclophosphamide is administered to the subject concurrent with each round of administration of the composition comprising a modified beta cell. In some embodiments, the cyclophosphamide is administered to the subject concurrent with each round of administration of the composition comprising a modified beta cell, and continued to be administered over the course of the subject's lifespan.

In some embodiments, the cyclophosphamide (e.g. a regimen of cyclophosphamide) is administered to the subject after administration of the composition comprising a modified beta cell to the subject. In some embodiments, at least one regimen of cyclophosphamide is administered to the subject after administration of the composition comprising a modified beta cell to the subject. In some embodiments, more than one regimen of cyclophosphamide is administered to the subject after administration of the composition comprising a modified beta cell to the subject. In some embodiments, the cyclophosphamide is administered to the subject only after administration of a first and/or second regimen of the composition comprising a modified beta cell to the subject. In some embodiments, the cyclophosphamide is administered between about 30 seconds and about 60 months after administration of the composition comprising a modified beta cell to the subject, such as between about 30 seconds and about 1 hour, between about 30 minutes and about 12 hours, between about 6 hours and about 1 day, between about 10 hours and about 5 days, between about 2 days and about 7 days, between about 5 days and about 14 days, between about 7 days and about 4 weeks, between about 2 weeks and about 10 weeks, or between about 10 weeks and about 60 months, after administration of the composition comprising a modified beta cell to the subject. In some embodiments, the cyclophosphamide is administered at least about 30 seconds after administration of the composition comprising a modified beta cell to the subject, such as least about any of 1 minute, 5 minutes, 15 minutes, 30 minutes, 1 hour, 2 hours, 4 hours, 6 hours, 8 hours, 10 hours, 12 hours, 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 12 months, 24 months, 60 months, or more, after administration of the composition comprising a modified beta cell to the subject. In some embodiments, the cyclophosphamide is administered less than about 60 months after administration of the composition comprising a modified beta cell to the subject, such less than about any of 24 months, 12 months, 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds prior to administration of the composition comprising a modified beta cell to the subject. In some embodiments, the cyclophosphamide is administered about 10 weeks, 9 weeks, 8 weeks, 7 weeks, 6 weeks, 5 weeks, 4 weeks, 3 weeks, 14 days, 13 days, 12 days, 11 days, 10 days, 9 days, 8 days, 7 days, 6 days, 5 days, 4 days, 3 days, 2 days, 1 day, 12 hours, 10 hours, 8 hours, 6 hours, 4 hours, 2 hours, 1 hour, 30 minutes, 15 minutes, 5 minutes 1 minute, or 30 seconds after administration of the composition comprising a modified beta cell to the subject. In some embodiments, the cyclophosphamide is administered to the subject after administration of the composition comprising a modified beta cell, and continued to be administered over the course of the subject's lifespan. In some embodiments, the cyclophosphamide is administered to the subject after each round of administration of the composition comprising a modified beta cell. In some embodiments, the cyclophosphamide is administered to the subject after each round of administration of the composition comprising a modified beta cell, and continued to be administered over the course of the subject's lifespan. In some embodiments, the cyclophosphamide is administered to the subject each day after the composition comprising a modified beta cell is administered to the subject. In some embodiments, the cyclophosphamide is administered to the subject each day for up to about 3 months after the composition comprising a modified beta cell is administered to the subject. In some embodiments, the cyclophosphamide is administered to the subject about four times each day after the composition comprising a modified beta cell is administered to the subject. In some embodiments, the cyclophosphamide is administered to the subject about four times each day after the composition comprising a modified beta cell is administered to the subject.

In some embodiments, a regimen and/or total daily dose of between about 100 mg/m² and about 1,000 mg/m² cyclophosphamide is administered to the subject, such as a regimen of between about 100 mg/m² and about 500 mg/m², between about 400 mg/m² and about 600 mg/m², or between about 500 mg/m² and about 1,000 mg/m². In some embodiments, a regimen of between about 400 mg/m² and about 600 mg/m² of cyclophosphamide is administered to the subject. In some embodiments, a regimen of greater than about 100 mg/m² of cyclophosphamide is administered to the subject, such as a regimen of greater than any of about 200 mg/m², 300 mg/m², 400 mg/m², 500 mg/m², 600 mg/m², 700 mg/m², 800 mg/m², 900 mg/m², 1,000 mg/m², or greater, of cyclophosphamide. In some embodiments, a regimen of less than about 1,000 mg/m² of cyclophosphamide is administered to the subject, such as a regimen of less than any of about, 900 mg/m², 800 mg/m², 700 mg/m², 600 mg/m², 500 mg/m², 400 mg/m², 300 mg/m², 200 mg/m², 100 mg/m², or less, of cyclophosphamide. In some embodiments, a regimen of about 500 mg/m² of cyclophosphamide is administered to the subject. In some embodiments, cyclophosphamide is administered to the subject intravenously.

In some embodiments, the subject is administered a regimen of cyclophosphamide and a regimen of fludarabine. In some embodiments, the subject is administered at least one regimen of cyclophosphamide and at least one regimen of fludarabine. In some embodiments, the subject is administered the at least one regimen of cyclophosphamide prior to, on the same day as, concurrent with, and/or after the at least one regimen of fludarabine. In some embodiments, the subject is administered the at least one regimen of fludarabine prior to, on the same day as, concurrent with, and/or after the at least one regimen of cyclophosphamide. In some embodiments, the subject is administered the at least one regimen of fludarabine prior to the at least one regimen of cyclophosphamide. In some embodiments, the subject is administered a regimen of fludarabine and a regimen of cyclophosphamide prior to the administration of the composition comprising a modified beta cell to the subject. In some embodiments, the subject is administered at least one regimen of fludarabine and at least one regimen of cyclophosphamide prior to the administration of the composition comprising a modified beta cell to the subject. In some embodiments, the subject is administered a regimen of about 30 mg/m² of fludarabine each day for 3 consecutive days about 2 days to about 7 days prior to the administration of the composition comprising a modified beta cell to the subject. In some embodiments, the subject is administered a regimen of about 500 mg/m² of cyclophosphamide each day for 3 consecutive days about 2 days to about 7 days prior to the administration of the composition comprising a modified beta cell to the subject. In some embodiments, the subject is administered a regimen of about 30 mg/m² of fludarabine and a regimen of about 500 mg/m² of cyclophosphamide each day for 3 consecutive days about 2 days to about 7 days prior to the administration of the composition comprising a modified beta cell to the subject. In some embodiments, the subject is administered a regimen of about 30 mg/m² of fludarabine each day for 2 consecutive days about 2 days to about 14 days prior the administration of the composition comprising a modified beta cell to the subject. In some embodiments, the subject is administered a regimen of about 500 mg/m² of cyclophosphamide each day for 2 consecutive days about 2 days to about 14 days prior the administration of the composition comprising a modified beta cell to the subject. In some embodiments, the subject is administered a regimen of about 30 mg/m² of fludarabine and about 500 mg/m² of cyclophosphamide each day for 2 consecutive days about 2 days to about 14 days prior the administration of the composition comprising a modified beta cell to the subject. In some embodiments, the subject is administered a regimen of about 30 mg/m² of fludarabine on day 5, day 4, and day 3 prior to the administration of the composition comprising a modified beta cell to the subject. In some embodiments, the subject is administered a regimen of about 500 mg/m² of cyclophosphamide on day 5, day 4, and day 3 prior to the administration of the composition comprising a modified beta cell to the subject. In some embodiments, the subject is administered a regimen of about 30 mg/m² of fludarabine and about 500 mg/m² of cyclophosphamide on day 5, day 4, and day 3 prior to the administration of the composition comprising a modified beta cell to the subject. In some embodiments, the fludarabine regimen is administered at a lower dose. In some embodiments, the cyclophosphamide regimen is administered at a lower dose. In some embodiments, the fludarabine regimen and the cyclophosphamide regimen is administered at a lower dose.

p. Additional Immunosuppressive Agents

Besides to the various categories of immunosuppressive agents described, additional immunosuppressive agents and regimens may be useful in the methods and uses provided herein.

In some embodiments, the one or more immunosuppressive agents comprise fingolimod hydrochloride. In some embodiments, the fingolimod hydrochloride (e.g. a regimen of fingolimod hydrochloride) is administered to the subject prior to, concurrent with, and/or after the administration of the composition comprising a modified beta cell to the subject. In some embodiments, at least one regimen of fingolimod hydrochloride is administered to the subject prior to, concurrent with, and/or after the administration of the composition comprising a modified beta cell to the subject.

In some embodiments, the one or more immunosuppressive agents comprise liposomal clodronate. In some embodiments, the liposomal clodronate (e.g. a regimen of liposomal clodronate) is administered to the subject prior to, concurrent with, and/or after the administration of the composition comprising a modified beta cell to the subject. In some embodiments, at least one regimen of liposomal clodronate is administered to the subject prior to, concurrent with, and/or after the administration of the composition comprising a modified beta cell to the subject.

In some embodiments, the one or more immunosuppressive agents comprise CTLA4-Ig. In some embodiments, the CTLA4-Ig (e.g. a regimen of CTLA4-Ig) is administered to the subject prior to, concurrent with, and/or after the administration of the composition comprising a modified beta cell to the subject. In some embodiments, at least one regimen of CTLA4-Ig is administered to the subject prior to, concurrent with, and/or after the administration of the composition comprising a modified beta cell to the subject.

In some embodiments, the one or more immunosuppressive agents comprise aryl hydrocarbon receptor (AhR) ligand 2-(1′H-indole-3′-carbonyl)-thiazole-4-carboxylic acid methyl ester (ITE). In some embodiments, the AhR ligand ITE (e.g. a regimen of AhR ligand ITE) is administered to the subject prior to, concurrent with, and/or after the administration of the composition comprising a modified beta cell to the subject. In some embodiments, at least one regimen of AhR ligand ITE is administered to the subject prior to, concurrent with, and/or after the administration of the composition comprising a modified beta cell to the subject.

In some embodiments, the one or more immunosuppressive agents comprise T1D autoantigen proinsulin. In some embodiments, the TID autoantigen proinsulin (e.g. a regimen of TID autoantigen proinsulin) is administered to the subject prior to, concurrent with, and/or after the administration of the composition comprising a modified beta cell to the subject. In some embodiments, at least one regimen of T1D autoantigen proinsulin is administered to the subject prior to, concurrent with, and/or after the administration of the composition comprising a modified beta cell to the subject.

In some embodiments, the one or more immunosuppressive agents comprise TGF-β1. In some embodiments, the TGF-β1 (e.g. a regimen of TGF-β1) is administered to the subject prior to, concurrent with, and/or after the administration of the composition comprising a modified beta cell to the subject. In some embodiments, at least one regimen of TGF-β1 is administered to the subject prior to, concurrent with, and/or after the administration of the composition comprising a modified beta cell to the subject.

In some embodiments, the one or more immunosuppressive agents comprise methotrexate. In some embodiments, the methotrexate (e.g. a regimen of methotrexate) is administered to the subject prior to, concurrent with, and/or after the administration of the composition comprising a modified beta cell to the subject. In some embodiments, at least one regimen of methotrexate is administered to the subject prior to, concurrent with, and/or after the administration of the composition comprising a modified beta cell to the subject.

In some embodiments, the one or more immunosuppressive agents comprise gold salts. In some embodiments, the gold salts (e.g. a regimen of gold salts) is administered to the subject prior to, concurrent with, and/or after the administration of the composition comprising a modified beta cell to the subject. In some embodiments, at least one regimen of gold salts is administered to the subject prior to, concurrent with, and/or after the administration of the composition comprising a modified beta cell to the subject.

In some embodiments, the one or more immunosuppressive agents comprise sulfasalazine. In some embodiments, the sulfasalazine (e.g. a regimen of sulfasalazine) is administered to the subject prior to, concurrent with, and/or after the administration of the composition comprising a modified beta cell to the subject. In some embodiments, at least one regimen of sulfasalazine is administered to the subject prior to, concurrent with, and/or after the administration of the composition comprising a modified beta cell to the subject.

In some embodiments, the one or more immunosuppressive agents comprise one or more anti-malarials. In some embodiments, the one or more anti-malarials (e.g. a regimen of one or more anti-malarials) are administered to the subject prior to, concurrent with, and/or after the administration of the composition comprising a modified beta cell to the subject. In some embodiments, at least one regimen of one or more anti-malarials is administered to the subject prior to, concurrent with, and/or after the administration of the composition comprising a modified beta cell to the subject.

In some embodiments, the one or more immunosuppressive agents comprise brequinar. In some embodiments, the brequinar (e.g. a regimen of brequinar) is administered to the subject prior to, concurrent with, and/or after the administration of the composition comprising a modified beta cell to the subject. In some embodiments, at least one regimen of brequinar is administered to the subject prior to, concurrent with, and/or after the administration of the composition comprising a modified beta cell to the subject.

In some embodiments, the one or more immunosuppressive agents comprise leflunomide. In some embodiments, the leflunomide (e.g. a regimen of leflunomide) is administered to the subject prior to, concurrent with, and/or after the administration of the composition comprising a modified beta cell to the subject. In some embodiments, at least one regimen of leflunomide is administered to the subject prior to, concurrent with, and/or after the administration of the composition comprising a modified beta cell to the subject.

In some embodiments, the one or more immunosuppressive agents comprise mizoribine. In some embodiments, the mizoribine (e.g. a regimen of mizoribine) is administered to the subject prior to, concurrent with, and/or after the administration of the composition comprising a modified beta cell to the subject. In some embodiments, at least one regimen of mizoribine is administered to the subject prior to, concurrent with, and/or after the administration of the composition comprising a modified beta cell to the subject.

In some embodiments, the one or more immunosuppressive agents comprise 15-deoxyspergualine. In some embodiments, the 15-deoxyspergualine (e.g. a regimen of 15-deoxyspergualine) is administered to the subject prior to, concurrent with, and/or after the administration of the composition comprising a modified beta cell to the subject. In some embodiments, at least one regimen of 15-deoxyspergualine is administered to the subject prior to, concurrent with, and/or after the administration of the composition comprising a modified beta cell to the subject.

In some embodiments, the one or more immunosuppressive agents comprise 6-mercaptopurine. In some embodiments, the 6-mercaptopurine (e.g. a regimen of 6-mercaptopurine) is administered to the subject prior to, concurrent with, and/or after the administration of the composition comprising a modified beta cell to the subject. In some embodiments, at least one regimen of 6-mercaptopurine is administered to the subject prior to, concurrent with, and/or after the administration of the composition comprising a modified beta cell to the subject.

### 3. Compositions and Formulations

In some aspects of the methods, combinations, kits, and uses provided herein, the one or more immunosuppressive agents can be administered in one or more compositions, e.g. a pharmaceutical composition containing one or more immunosuppressive agents.

In some embodiments, the pharmaceutical composition provided herein further includes a pharmaceutically acceptable excipient or carrier. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polysorbates (TWEEN™), poloxamers (PLURONICS™) or polyethylene glycol (PEG). In some embodiments, the pharmaceutical composition includes a pharmaceutically acceptable buffer (e.g. neutral buffer saline or phosphate buffered saline). In some embodiments, the pharmaceutical composition can contain one or more excipients for modifying, maintaining or preserving, for example, the pH, osmolarity, viscosity, clarity, color, isotonicity, odor, sterility, stability, rate of dissolution or release, adsorption, or penetration of the composition.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject.

The pharmaceutical composition in some embodiments contains one or more immunosuppressive agents, as described herein in amounts effective to treat or prevent the beta cell associated disease or disorder, such as a therapeutically effective or prophylactically effective amount. In some embodiments, the pharmaceutical composition contains one or more immunosuppressive agents as described herein in amounts effective to treat or prevent the beta cell associated disease or disorder, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In some embodiments, one or more immunosuppressive agents as described herein are administered using standard administration techniques, formulations, and/or devices. In some embodiments, one or more immunosuppressive agents as described herein are administered using standard administration techniques, formulations, and/or devices. Provided are formulations and devices, such as syringes and vials, for storage and administration of the compositions. One or more immunosuppressive agents can be administered via localized injection, including catheter administration, systemic injection, localized injection, intravenous injection, or parenteral administration. When administering a therapeutic composition (e.g. a pharmaceutical composition containing one or more immunosuppressive agents), it will generally be formulated in a unit dosage injectable form (solution, suspension, emulsion).

Formulations include those for intravenous, intraperitoneal, or subcutaneous, administration. In some embodiments, the one or more immunosuppressive agents are administered parenterally. The term “parenteral,” as used herein, includes intravenous, intramuscular, subcutaneous, rectal, vaginal, and intraperitoneal administration. In some embodiments, the one or more immunosuppressive agents are administered to a subject using peripheral systemic delivery by intravenous, intraperitoneal, or subcutaneous injection.

Compositions in some embodiments are provided as sterile liquid preparations, e.g. isotonic aqueous solutions, suspensions, emulsions, or dispersions, which may in some aspects be buffered to a selected pH. Liquid compositions are somewhat more convenient to administer, especially by injection. Liquid compositions can comprise carriers, which can be a solvent or dispersing medium containing, for example, water, saline, phosphate buffered saline, polyol (for example, glycerol, propylene glycol, liquid polyethylene glycol) and suitable mixtures thereof. Sterile injectable solutions can be prepared by incorporating the one or more immunosuppressive agents in a solvent, such as in admixture with a suitable carrier, diluent, or excipient such as sterile water, physiological saline, glucose, dextrose, or the like.

In some embodiments, the pharmaceutical composition can be formulated for administration by any route known to those of skill in the art including intramuscular, intravenous, intradermal, intralesional, intraperitoneal injection, subcutaneous, intratumoral, epidural, nasal, oral, vaginal, rectal, topical, local, otic, inhalational, buccal (e.g. sublingual), and transdermal administration or any route. In some embodiments, other modes of administration also are contemplated. In some embodiments, the administration is by bolus infusion, by injection, e.g. intravenous or subcutaneous injections, intraocular injection, periocular injection, subretinal injection, intravitreal injection, trans-septal injection, subscleral injection, intrachoroidal injection, intracameral injection, subconjunctival injection, sub-Tenon's injection, retrobulbar injection, peribulbar injection, or posterior juxtascleral delivery. In some embodiments, administration is by parenteral, intrapulmonary, and intranasal, and, if desired for local treatment, intralesional administration. Parenteral infusions include intramuscular, intravenous, intraarterial, intraperitoneal, or subcutaneous administration. In some embodiments, the administration is via the portal vein. In some embodiments, the administration is by injection into the intramuscular space forearm of the subject.

In some embodiments, compositions also can be administered with other biologically active agents, either sequentially, intermittently or in the same composition. In some embodiments, administration also can include controlled release systems including controlled release formulations and device-controlled release, such as by means of a pump. In some embodiments, the administration is oral. In some embodiments, the administration is intravenous.

In some embodiments, the one or more immunosuppressive agents are typically formulated and administered in unit dosage forms or multiple dosage forms. Each unit dose contains a predetermined quantity of therapeutically active immunosuppressive agent, sufficient to produce the desired therapeutic effect, in association with the required pharmaceutical carrier, vehicle or diluent. In some embodiments, unit dosage forms, include, but are not limited to, tablets, capsules, pills, powders, granules, sterile parenteral solutions or suspensions, and oral solutions or suspensions, and oil water emulsions containing suitable quantities of one or more immunosuppressive agents. Unit dose forms can be contained ampoules and syringes or individually packaged tablets or capsules. Unit dose forms can be administered in fractions or multiples thereof. In some embodiments, a multiple dose form is a plurality of identical unit dosage forms packaged in a single container to be administered in segregated unit dose form. Examples of multiple dose forms include vials, bottles of tablets or capsules or bottles of pints or gallons.

### B. Tapering Immunosuppressive Agents

In some aspects, the immunosuppressive regimens provided herein (e.g. immunosuppression protocols of any of the one or more immunosuppressive agents provided herein may be tapered, or reduced. Tapering of an immunosuppression regimen may be beneficial for returning natural immunity and reducing drug-related toxicity in a subject. However, there could be potential risks associated with tapering, and thus, in some exemplary aspects, it may be necessary to gradually taper an immunosuppression regimen by maintaining some immunosuppression for many years and/or over time entire lifespan of the subject.

In some embodiments, the methods provided herein further comprise tapering the administration of the one or more immunosuppressive agents to the subject. In some embodiments, the tapering comprises gradually reducing the amount of the one or more immunosuppressive agents that are administered to the subject. In some embodiments, the one or more immunosuppressive agents are tapered after the administration of the composition comprising a modified beta cell to the subject. In some embodiments, the methods provided herein do not further comprise tapering the administration of the one or more immunosuppressive agents to the subject.

In some embodiments, the one or more immunosuppression agents can be tapered over a period of between about 1 day and about 90 years after the administration of the composition comprising a modified beta cell to the subject, such as between about 1 day and about 12 months, between about 6 months and about 2 years, between about 12 months and about 20 years, between about 10 years and about 50 years, or between about 30 years and about 90 years. In some embodiments, the one or more immunosuppressive agents can be tapered over a period of less than about 90 years, such as less than about any of 80 years, 70 years, 60 years, 50 years, 40 years, 30 years, 20 years, 10 years, 5 years, 2 years, 12 months, 10 months, 8 months, 6 months, 4 months, 2 months, 1 month, 15 days, 10 days, 5 days, 1 day, or less. In some embodiments, the one or more immunosuppressive agents can be tapered over a period of greater than about 1 day, such as greater than about any of 5 days, 10 days, 15 days, 1 month, 2 months, 4 months, 6 months, 8 months, 10 months, 12 months, 2 years, 5 years, 10 years, 20 years, 30 years, 40 years, 50 years, 60 years, 70 years, 80 years, 90 years, or greater. In some embodiments, the tapering does not result in complete withdraw of the administration of the one or more immunosuppressive agents. In some embodiments, the tapering is completed when the subject is not administered any additional one or more immunosuppressive agents.

### III. Engineered Islet Cells and Generation Thereof

Provided herein are compositions comprising an engineered islet that includes islet cells that comprise one or more modifications (termed “engineered islet cell” or “modified islet cell”) that comprises a modification that regulates the expression of one or more target polynucleotide sequences, such as regulates the expression of one or more MHC class I molecules, one or more MHC class II molecules, or one or more MHC class I molecules and one or more MHC class II molecules.

In some embodiments, the provided engineered islets, also includes a modification to modulate (e.g., increase) expression of one or more tolerogenic factor. In some embodiments, the modulation of expression of the tolerogenic factor (e.g., increased expression), and the modulation of expression of the one or more MHC class I molecules and/or one or more MHC class II molecules (e.g., reduced or eliminated expression) is relative to the amount of expression of said molecule(s) in a cell that does not comprise the modification(s), such as a control cell. In some embodiments, the modulation of expression is relative to the amount of expression of said molecule(s) in a wild-type cell. In some embodiments, the control or wild-type cell is an islet cell that has not been engineered with the modifications. In some embodiments, modulation of expression of the tolerogenic factor (e.g., increased expression), and the modulation of expression of the one or more MHC class I molecules and/or one or more MHC class II molecules (e.g., reduced or eliminated expression) is relative to the amount of expression of said molecule(s) in a control or wild-type cell of the same cell type that does comprise not the modification(s). In some embodiments, the control or wild-type cell does not express the one or more tolerogenic factor, the one or more MHC class I molecules, and/or the one or more MHC class II molecules. In some embodiments, it is understood that where the control or wild-type cell does not express the tolerogenic factor, the provided engineered islet cell includes a modification to overexpress the one or more tolerogenic factor or increase the expression of the one or more tolerogenic factor from 0%. It is understood that if the islet cell prior to the engineering does not express a detectable amount of the tolerogenic factor, then a modification that results in any detectable amount of an expression of the tolerogenic factor is an increase in the expression compared to the similar beta cell that does not contain the modifications.

In some embodiments, the provided engineered islets includes a modification to increase expression of one or more tolerogenic factors. In some embodiments, the tolerogenic factor is one or more of DUX4, B2M-HLA-E, CD35, CD52, CD16, CD52, CD47, CD46, CD55, CD59, CD27, CD200, HLA-C, HLA-E, HLA-E heavy chain, HLA-G, PD-L1, IDO1, CTLA4-Ig, Cl-Inhibitor, IL-10, IL-35, FASL, CCL21, MFGE8, SERPINB9, CD35, IL-39, CD16 Fe Receptor, IL15-RF, and H2-M3 (including any combination thereof). In some embodiments, the tolerogenic factor is one or more of CD47, PD-L1, HLA-E or HLA-G, CCL21, FasL, Serpinb9, CD200, and Mfge8 (including any combination thereof). In some embodiments, the modification to increase expression of one or more tolerogenic factors is or includes increased expression of CD47. In some embodiments, the modification to increase expression of one or more tolerogenic factors is or includes increased expression of PD-L1. In some embodiments, the modification to increase expression of one or more tolerogenic factors is or includes increased expression of HLA-E. In some embodiments, the modification to increase expression of one or more tolerogenic factors is or includes increased expression of HLA-G. In some embodiments, the modification to increase expression of one or more tolerogenic factors is or includes increased expression of CCL21, PD-L1, FasL, Serpinb9, H2-M3 (HLA-G), CD47, CD200, and Mfge8.

In some embodiments, the engineered islets includes one or more modifications, such as genomic modifications, that reduce expression of one or more MHC class I molecules and a modification that increases expression of CD47. In other words, the engineered islets comprise exogenous CD47 proteins and exhibit reduced or silenced surface expression of one or more one or more MHC class I molecules. In some embodiments, the engineered islets includes one or more genomic modifications that reduce expression of one or more MHC class II molecules and a modification that increases expression of CD47. In some instances, the engineered islets comprise exogenous CD47 nucleic acids and proteins and exhibit reduced or silenced surface expression of one or more MHC class I molecules. In some embodiments, the engineered islets includes one or more genomic modifications that reduce or eliminate expression of one or more MHC class II molecules, one or more genomic modifications that reduce or eliminate expression of one or more MHC class II molecules, and a modification that increases expression of CD47. In some embodiments, the engineered islets comprise exogenous CD47 proteins, exhibit reduced or silenced surface expression of one or more MHC class I molecules and exhibit reduced or lack surface expression of one or more MHC class II molecules. In many embodiments, the engineered islets is a B2M indel/indel, CIITAindel/indel, CD47tg cell.

In some embodiments, the engineered islets elicits a reduced level of immune activation or no immune activation upon administration to a recipient subject. In some embodiments, the engineered islets elicits a reduced level of systemic TH1 activation or no systemic TH1 activation in a recipient subject. In some embodiments, the engineered islets elicits a reduced level of immune activation of peripheral blood mononuclear cells (PBMCs) or no immune activation of PBMCs in a recipient subject. In some embodiments, the engineered islets elicits a reduced level of donor-specific IgG antibodies or no donor specific IgG antibodies against the cells upon administration to a recipient subject. In some embodiments, the engineered islets elicits a reduced level of IgM and IgG antibody production or no IgM and IgG antibody production against the cells in a recipient subject. In some embodiments, the engineered islets elicits a reduced level of cytotoxic T cell killing of the cells upon administration to a recipient subject.

In some embodiments, the engineered islets provided herein comprises a “suicide gene” or “suicide switch”. A suicide gene or suicide switch can be incorporated to function as a “safety switch” that can cause the death of the engineered islets, such as after the engineered islets is administered to a subject and if the engineered islets should grow and divide in an undesired manner. The “suicide gene” ablation approach includes a suicide gene in a gene transfer vector encoding a protein that results in cell killing only when activated by a specific compound. A suicide gene may encode an enzyme that selectively converts a nontoxic compound into highly toxic metabolites. The result is specifically eliminating cells expressing the enzyme. In some embodiments, the suicide gene is the herpesvirus thymidine kinase (HSV-tk) gene and the trigger is ganciclovir. In other embodiments, the suicide gene is the Escherichia coli cytosine deaminase (EC-CD) gene and the trigger is 5-fluorocytosine (5-FC) (Barese et al, Mol. Therap. 20(10): 1932-1943 (2012), Xu et al, Cell Res. 8:73-8 (1998), both incorporated herein by reference in their entirety).

In other embodiments, the suicide gene is an inducible Caspase protein. An inducible Caspase protein comprises at least a portion of a Caspase protein capable of inducing apoptosis. In preferred embodiments, the inducible Caspase protein is iCasp9. It comprises the sequence of the human FK506-binding protein, FKBP12, with an F36V mutation, connected through a series of amino acids to the gene encoding human caspase 9. FKBP12-F36V binds with high affinity to a small-molecule dimerizing agent, API 903. Thus, the suicide function of iCasp9 in the instant invention is triggered by the administration of a chemical inducer of dimerization (CID). I n some embodiments, the CID is the small molecule drug API 903. Dimerization causes the rapid induction of apoptosis. (See WO2011146862; Stasi et al, N. Engl. J. Med 365; 18 (2011); Tey et al, Biol. Blood Marrow Transplant. 13:913-924 (2007), each of which are incorporated by reference herein in their entirety.)

Inclusion of a safety switch or suicide gene allows for controlled killing of the cells in the event of cytotoxicity or other negative consequences to the recipient, thus increasing the safety of cell-based therapies, including those using tolerogenic factors.

In some embodiments, a safety switch can be incorporated into, such as introduced, into the engineered islets provided herein to provide the ability to induce death or apoptosis of the engineered islets containing the safety switch, for example if the cells grow and divide in an undesired manner or cause excessive toxicity to the host. Thus, the use of safety switches enables one to conditionally eliminate aberrant cells in vivo and can be a critical step for the application of cell therapies in the clinic. Safety switches and their uses thereof are described in, for example, Duzgune, Origins of Suicide Gene Therapy (2019); Duzgune (eds), Suicide Gene Therapy. Methods in Molecular Biology, vol. 1895 (Humana Press, New York, NY) (for HSV-tk, cytosine deaminase, nitroreductase, purine nucleoside phosphorylase, and horseradish peroxidase); Zhou and Brenner, Exp Hematol 44(11):1013-1019 (2016) (for iCaspase9); Wang et al., Blood 18(5):1255-1263 (2001) (for huEGFR); U.S. Patent Application Publication No. 20180002397 (for HER1); and Philip et al., Blood124(8):1277-1287 (2014) (for RQR8).

In some embodiments, the safety switch can cause cell death in a controlled manner, for example, in the presence of a drug or prodrug or upon activation by a selective exogenous compound. In some embodiments, the safety switch is selected from the group consisting of herpes simplex virus thymidine kinase (HSV-tk), cytosine deaminase (CyD), nitroreductase (NTR), purine nucleoside phosphorylase (PNP), horseradish peroxidase, inducible caspase 9 (iCasp9), rapamycin-activated caspase 9 (rapaCasp9), CCR4, CD16, CD19, CD20, CD30, EGFR, GD2, HER1, HER2, MUC1, PSMA, and RQR8.

In some embodiments, the safety switch may be a transgene encoding a product with cell killing capabilities when activated by a drug or prodrug, for example, by turning a non-toxic prodrug to a toxic metabolite inside the cell. In these embodiments, cell killing is activated by contacting a engineered islets with the drug or prodrug. In some cases, the safety switch is HSV-tk, which converts ganciclovir (GCV) to GCV-triphosphate, thereby interfering with DNA synthesis and killing dividing cells. In some cases, the safety switch is CyD or a variant thereof, which converts the antifungal drug 5-fluorocytosine (5-FC) to cytotoxic 5-fluorouracil (5-FU) by catalyzing the hydrolytic deamination of cytosine into uracil. 5-FU is further converted to potent anti-metabolites (5-FdUMP, 5-FdUTP, 5-FUTP) by cellular enzymes. These compounds inhibit thymidylate synthase and the production of RNA and DNA, resulting in cell death. In some cases, the safety switch is NTR or a variant thereof, which can act on the prodrug CB 1954 via reduction of the nitro groups to reactive N-hydroxylamine intermediates that are toxic in proliferating and nonproliferating cells. In some cases, the safety switch is PNP or a variant thereof, which can turn prodrug 6-methylpurine deoxyriboside or fludarabine into toxic metabolites to both proliferating and nonproliferating cells. In some cases, the safety switch is horseradish peroxidase or a variant thereof, which can catalyze indole-3-acetic acid (IAA) to a potent cytotoxin and thus achieve cell killing.

In some embodiments, the safety switch may be an iCasp9. Caspase 9 is a component of the intrinsic mitochondrial apoptotic pathway which, under physiological conditions, is activated by the release of cytochrome C from damaged mitochondria. Activated caspase 9 then activates caspase 3, which triggers terminal effector molecules leading to apoptosis. The iCasp9 may be generated by fusing a truncated caspase 9 (without its physiological dimerization domain or caspase activation domain) to a FK506 binding protein (FKBP), FKBP12-F36V, via a peptide linker. The iCasp9 has low dimer-independent basal activity and can be stably expressed in host cells (e.g., human T cells) without impairing their phenotype, function, or antigen specificity. However, in the presence of chemical inducer of dimerization (CID), such as rimiducid (AP1903), AP20187, and rapamycin, iCasp9 can undergo inducible dimerization and activate the downstream caspase molecules, resulting in apoptosis of cells expressing the iCasp9. See, e.g., PCT Application Publication No. WO2011/146862; Stasi et al., N. Engl. J. Med. 365; 18 (2011); Tey et al., Biol. Blood Marrow Transplant 13:913-924 (2007). In particular, the rapamycin inducible caspase 9 variant is called rapaCasp9. See Stavrou et al., Mal. Ther. 26(5):1266-1276 (2018). Thus, iCasp9 can be used as a safety switch to achieve controlled killing of the host cells.

In some embodiments, the safety switch may be a membrane-expressed protein which allows for cell depletion after administration of a specific antibody to that protein. Safety switches of this category may include, for example, one or more transgene encoding CCR4, CD16, CD19, CD20, CD30, EGFR, GD2, HER1, HER2, MUC1, PSMA, or RQR8 for surface expression thereof. These proteins may have surface epitopes that can be targeted by specific antibodies. In some embodiments, the safety switch comprises CCR4, which can be recognized by an anti-CCR4 antibody. Non-limiting examples of suitable anti-CCR4 antibodies include mogamulizumab and biosimilars thereof. In some embodiments, the safety switch comprises CD16 or CD30, which can be recognized by an anti-CD16 or anti-CD30 antibody. Non-limiting examples of such anti-CD16 or anti-CD30 antibody include AFM13 and biosimilars thereof. In some embodiments, the safety switch comprises CD19, which can be recognized by an anti-CD19 antibody. Non-limiting examples of such anti-CD19 antibody include MOR208 and biosimilars thereof. In some embodiments, the safety switch comprises CD20, which can be recognized by an anti-CD20 antibody. Non-limiting examples of such anti-CD20 antibody include obinutuzumab, ublituximab, ocaratuzumab, rituximab, rituximab-Rllb, and biosimilars thereof. Cells that express the safety switch are thus CD20-positive and can be targeted for killing through administration of an anti-CD20 antibody as described. In some embodiments, the safety switch comprises EGFR, which can be recognized by an anti-EGFR antibody. Non-limiting examples of such anti-EGFR antibody include tomuzotuximab, RO5083945 (GA201), cetuximab, and biosimilars thereof. In some embodiments, the safety switch comprises GD2, which can be recognized by an anti-GD2 antibody. Non-limiting examples of such anti-GD2 antibody include Hul4.18K322A, Hul4.18-IL2, Hu3F8, dinituximab, c.60C3-Rllc, and biosimilars thereof.

In some embodiments, the safety switch may be an exogenously administered agent that recognizes one or more tolerogenic factor on the surface of the engineered islets. In some embodiments, the exogenously administered agent is an antibody directed against or specific to a tolerogenic agent, e.g., an anti-CD47 antibody. By recognizing and blocking a tolerogenic factor on engineered islets, an exogenously administered antibody may block the immune inhibitory functions of the tolerogenic factor thereby re-sensitizing the immune system to the engineered islets. For instance, for a engineered islets that overexpresses CD47 an exogenously administered anti-CD47 antibody may be administered to the subject, resulting in masking of CD47 on the engineered islets and triggering of an immune response to the engineered islets. In some embodiments, the anti-CD47 antibody is Magrolimab.

In some embodiments, the safety switch comprises an anti-CD47 antibody. In some embodiments, the anti-CD47 antibody is Magrolimab. In some embodiments, the safety switch is Magrolimab.

In some embodiments, the method further comprises introducing an expression vector comprising an inducible suicide switch into the cell.

In some embodiments, the tolerogenic factor is CD47 and the cell includes an exogenous polynucleotide encoding a CD47 protein. In some embodiments, the cell expresses an exogenous CD47 polypeptide.

In some embodiments, a method disclosed herein comprises administering to a subject in need thereof a CD47-SIRPα blockade agent, wherein the subject was previously administered a engineered islets engineered to express an exogenous CD47 polypeptide. In some embodiments, the CD47-SIRPα blockade agent comprises a CD47-binding domain. In some embodiments, the CD47-binding domain comprises signal regulatory protein alpha (SIRPα) or a fragment thereof. In some embodiments, the CD47-SIRPα blockade agent comprises an immunoglobulin G (IgG) Fc domain. In some embodiments, the IgG Fc domain comprises an IgG1 Fc domain. In some embodiments, the IgG1 Fc domain comprises a fragment of a human antibody. In some embodiments, the CD47-SIRPα blockade agent is selected from the group consisting of TTI-621, TTI-622, and ALX148. In some embodiments, the CD47-SIRPα blockade agent is TTI-621, TTI-622, and ALX148. In some embodiments, the CD47-SIRPα blockade agent is TTI-622. In some embodiments, the CD47-SIRPα blockade agent is ALX148. In some embodiments, the IgG Fc domain comprises an IgG4 Fc domain. In some embodiments, the CD47-SIRPα blockade agent is an antibody. In some embodiments, the antibody is selected from the group consisting of MIAP410, B6H12, and Magrolimab. In some embodiments, the antibody is MIAP410. In some embodiments, the antibody is B6H12. In some embodiments, the antibody is Magrolimab. In some embodiments, the antibody is selected from the group consisting of AO-176, IBI188 (letaplimab), STI-6643, and ZL-1201. In some embodiments, the antibody is AO-176 (Arch). In some embodiments, the antibody is IBI188 (letaplimab) (Innovent). In some embodiments, the antibody is STI-6643 (Sorrento). In some embodiments, the antibody is ZL-1201 (Zai).

In some embodiments, useful antibodies or fragments thereof that bind CD47 can be selected from a group that includes magrolimab ((Hu5F9-G4)) (Forty Seven, Inc.; Gilead Sciences, Inc.), urabrelimab, CC-90002 (Celgene; Bristol-Myers Squibb), IBI-188 (Innovent Biologics), IBI-322 (Innovent Biologics), TG-1801 (TG Therapeutics; also known as NI-1701, Novimmune SA), ALX148 (ALX Oncology), TJ011133 (also known as TJC4, I-Mab Biopharma), FA3M3, ZL-1201 (Zai Lab Co., Ltd), AK117 (Akesbio Australia Pty, Ltd.), AO-176 (Arch Oncology), SRF231 (Surface Oncology), GenSci-059 (GeneScience), C47B157 (Janssen Research and Development), C47B161 (Janssen Research and Development), C47B167 (Janssen Research and Development), C47B222 (Janssen Research and Development), C47B227 (Janssen Research and Development), Vx-1004 (Corvus Pharmaceuticals), HMBD004 (Hummingbird Bioscience Pte Ltd), SHR-1603 (Hengrui), AMMS4-G4 (Beijing Institute of Biotechnology), RTX-CD47 (University of Groningen), and IMC-002. (Samsung Biologics; ImmuneOncia Therapeutics). In some embodiments, the antibody or fragment thereof does not compete for CD47 binding with an antibody selected from a group that includes magrolimab, urabrelimab, CC-90002, IBI-188, IBI-322, TG-1801 (NI-1701), ALX148, TJ011133, FA3M3, ZL1201, AK117, AO-176, SRF231, GenSci-059, C47B157, C47B161, C47B167, C47B222, C47B227, Vx-1004, HMBD004, SHR-1603, AMMS4-G4, RTX-CD47, and IMC-002. In some embodiments, the antibody or fragment thereof competes for CD47 binding with an antibody selected from magrolimab, urabrelimab, CC-90002, IBI-188, IBI-322, TG-1801 (NI-1701), ALX148, TJ011133, FA3M3, ZL1201, AK117, AO-176, SRF231, GenSci-059, C47B157, C47B161, C47B167, C47B222, C47B227, Vx-1004, HMBD004, SHR-1603, AMMS4-G4, RTX-CD47, and IMC-002. In some embodiments, the antibody or fragment thereof that binds CD47 is selected from a group that includes a single-chain Fv fragment (scFv) against CD47, a Fab against CD47, a VHH nanobody against CD47, a DARPin against CD47, and variants thereof. In some embodiments, the scFv against CD47, a Fab against CD47, and variants thereof are based on the antigen binding domains of any of the antibodies selected from a group that includes magrolimab, urabrelimab, CC-90002, IBI-188, IBI-322, TG-1801 (NI-1701), ALX148, TJ011133, FA3M3, ZL1201, AK117, AO-176, SRF231, GenSci-059, C47B157, C47B161, C47B167, C47B222, C47B227, Vx-1004, HMBD004, SHR-1603, AMMS4-G4, RTX-CD47, and IMC-002.

In some embodiments, the CD47 antagonist provides CD47 blockade. Methods and agents for CD47 blockade are described in PCT/US2021/054326, which is incorporated by reference in its entirety.

In some embodiments, the engineered islets is derived from a source cell already comprising one or more of the desired modifications. In some embodiments, in view of the teachings provided herein one of ordinary skill in the art will readily appreciate how to assess what modifications are required to arrive at the desired final form of a engineered islets and that not all reduced or increased levels of target components are achieved via active engineering. In some embodiments, the modifications of the engineered islets may be in any order, and not necessarily the order listed in the descriptive language provided herein.

Once altered, the presence of expression of any of the molecule described herein can be assayed using known techniques, such as Western blots, ELISA assays, FACS assays, flow cytometry, and the like.

### A. Targets Having Reduced Expression Genes

In some embodiments, the engineered islets comprise a modification (e.g. genetic modifications) of one or more target polynucleotide or protein sequences (also interchangeably referred to as a target gene) that regulate (e.g. reduce or eliminate) the expression of one or more of: one or more MHC class I molecules, one or more MHC class II molecules, MIC-A, MIC-B, TXIP, CTLA-4 and/or PD-1. In some embodiments, the engineered islets comprise a modification of one or more gene that regulates (e.g. reduce or eliminate) one or more MHC class I molecules and/or one or more MHC class II molecules. In some embodiments, the one or more MHC class I molecules and/or one or more MHC class II molecules is any one or more of HLA-A, HLA-B, HLA-C, HLA-DP, HLA-DM, HLA-DOA, HLA-DOB, HLA-DQ and/or HLA-DR. In some embodiments, the modification to the target gene is a modification that reduces or eliminates any one or more of B2M, TAP I, NLRC5, CIITA, RFX5, RFXANK, RFXAP, NFY-A, NFY-B or NFY-C. In some embodiments, the engineered islets comprise a modification that reduces or eliminates expression of one or more of B2M, TAP I, NLRC5, CIITA, RFX5, RFXANK, RFXAP, NFY-A, NFY-B, NFY-C, MIC-A, MIC-B, TXIP, CTLA-4 and/or PD-1. Any of a variety of methods known to a skilled artisan can be used to reduce or eliminate expression of any such target genes, including any of variety of known gene editing technologies.

In some embodiments, the provided the engineered islets comprise a modification (e.g. genetic modifications) of one or more target polynucleotide or protein sequences (also interchangeably referred to as a target gene) that regulate (e.g. reduce or eliminate) the expression of either one or more MHC class I molecules, one or more MHC class II molecules, or one or more MHC class I molecules and one or more MHC class II molecules. In some embodiments, the beta cell to be modified is an unmodified cell or non-modified cell (e.g., control cell) or a wild-type beta cell, such as non-engineered islets, that has not previously been introduced with the one or more modifications. In some embodiments, a genetic editing system is used to modify one or more target polynucleotide sequences that regulate (e.g. reduce or eliminate) the expression of either one or more MHC class I molecules, one or more MHC class II molecules, or one or more MHC class I molecules and one or more MHC class II molecules. In certain embodiments, the genome of the cell has been altered to reduce or delete components required or involved in facilitating HLA expression, such as expression of one or more MHC class I molecules and/or one or more MHC class II molecules on the surface of the cell. For instance, in some embodiments, expression of a beta-2-microgloublin (B2M), a component of one or more MHC class I molecules, is reduced or eliminated in the cell, thereby reducing or elimination the protein expression (e.g. cell surface expression) of one or more MHC class I molecules by the modified cell. Thus, in some embodiments, expression can be reduced via a gene, and/or function thereof, RNA expression and function, protein expression and function, localization (such as cell surface expression), and longevity.

In some embodiments, an MHC in humans is also called a human leukocyte antigen (HLA). For instance, a human MHC class I is also known as an HLA class I and a human MHC class II is also known as an HLA class II. Thus, reference to MHC is intended to include the corresponding human HLA molecules, unless stated otherwise.

In some embodiments, reduced expression of a target is such that expression in a engineered islets is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a corresponding level of expression (e.g., protein expression compared with protein expression) of the target in a source cell prior to being modified to reduce expression of the target. In some embodiments, reduced expression of a target is such that expression in a engineered islets is reduced to a level that is about 60% or less (such as any of about 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a corresponding level of expression (e.g., protein expression compared with protein expression) of the target in a reference cell or a reference cell population (such as a cell or population of the same cell type or a cell having reduced or eliminated immunogenic response). In some embodiments, reduced expression of a target is such that expression in a engineered islets is reduced to a level that is at or less than a measured level of expression (such as a level known to exhibit reduced or eliminated immunogenic response due to the presence of the target). In some embodiments, the level of a target is assessed in a engineered islets, a reference cell, or reference cell population in a stimulated or non-stimulated state. In some embodiments, the level of a target is assessed in a engineered islets, a reference cell, or reference cell population in a stimulated state such that the target is expressed (or will be if it is a capability of the cell in response to the stimulus). In some embodiments, the stimulus represents an in vivo stimulus.

In some embodiments, the provided a engineered islets comprise a modification, such as a genetic modification, of one or more target polynucleotide sequences (also interchangeably referred to as a target gene) that regulate (e.g., reduce or eliminate) the expression of either one or more MHC class I molecules, one or more MHC class II molecules, or one or more MHC class I molecules and one or more MHC class II molecules. In some embodiments, an MHC in humans is also called a human leukocyte antigen. For instance, a human MHC class I molecule is also known as an HLA class I molecule and a human MHC class II molecules is also known as an HLA class II molecule. In some embodiments, the cell to be modified or modified is an unmodified cell or non-modified cell (e.g., control or wild-type cell) that has not previously been introduced with the one or more modifications. In some embodiments, a genetic editing system is used to modify one or more target polynucleotide sequences that regulate the expression of either one or more MHC class I molecules, one or more MHC class II molecules, or one or more MHC class I molecules and one or more MHC class II molecules. In certain embodiments, the genome of the cell has been altered to reduce or delete components require or involved in facilitating HLA expression, such as expression of one or more MHC class I molecules and/or one or more MHC class II molecules on the surface of the cell. For instance, in some embodiments, expression of a beta-2-microgloublin (B2M), a component of one or more MHC class I molecules, is reduced or eliminated in the cell, thereby reducing or elimination the protein expression (e.g. cell surface expression) of one or more MHC class I molecules by the modified cell.

In some embodiments, any of the described modifications in the engineered islets that regulate (e.g. reduce or eliminate) expression of one or more target polynucleotide or protein in the modified cell may be combined together with one or more modifications to overexpress a polynucleotide (e.g. tolerogenic factor, such as CD47) described in Section IIB.

In some embodiments, reduction of one or more MHC class I molecules and/or one or more MHC class II molecules expression can be accomplished, for example, by one or more of the following: (1) targeting the polymorphic HLA alleles (HLA-A, HLA-B, HLA-C) and one or more MHC class II molecules genes directly; (2) removal of B2M, which will reduce surface trafficking of all MHC class I molecules; and/or (3) deletion of one or more components of the MHC enhanceosomes, such as LRC5, RFX-5, RFXANK, RFXAP, IRF1, NF-Y (including NFY-A, NFY-B, NFY-C), and CIITA that are critical for HLA expression.

In certain embodiments, HLA expression is interfered with. In some embodiments, HLA expression is interfered with by targeting individual HLAs (e.g., knocking out expression of HLA-A, HLA-B and/or HLA-C), targeting transcriptional regulators of HLA expression (e.g., knocking out expression of NLRC5, CIITA, RFX5, RFXAP, RFXANK, NFY-A, NFY-B, NFY-C and/or IRF-1), blocking surface trafficking of one or more MHC class I molecules (e.g., knocking out expression of B2M and/or TAP 1), and/or targeting with HLA-RAZOR (see, e.g., WO2016183041).

The human leukocytes antigen (HLA) complex is synonymous with human MHC. In some embodiments, the engineered islets disclosed herein is a human cell. In certain aspects, the engineered islets disclosed herein does not express one or more human leukocyte antigens (e.g., HLA-A, HLA-B and/or HLA-C) corresponding to one or more MHC class I molecules and/or one or more MHC class II molecules and are thus characterized as being hypoimmunogenic. For example, in certain aspects, the engineered islets disclosed herein has been modified such that the cell does not express or exhibit reduced expression of one or more of the following MHC class I molecules: HLA-A, HLA-B and HLA-C. In some embodiments, one or more of HLA-A, HLA-B and HLA-C may be “knocked-out” of a cell. A cell that has a knocked-out HLA-A gene, HLA-B gene, and/or HLA-C gene may exhibit reduced or eliminated expression of each knocked-out gene.

In certain embodiments, the expression of one or more MHC class I molecules and/or one or more MHC class II molecules is modulated by targeting and deleting a contiguous stretch of genomic DNA, thereby reducing or eliminating expression of a target gene selected from the group consisting of B2M, CIITA, and NLRC5.

In some embodiments, the provided engineered islets comprise a modification, such as a genetic modification, of one or more target polynucleotide sequence that regulate one or more MHC class I. Exemplary methods for reducing expression of one or more MHC class I molecules are described in sections below. In some embodiments, the targeted polynucleotide sequence is one or both of B2M and NLRC5. In some embodiments, the engineered islets comprise a genetic editing modification to the B2M gene. In some embodiments, the engineered islets comprise a genetic editing modification to the NLRC5 gene. In some embodiments, the engineered islets comprise genetic editing modifications to the B2M and CIITA genes.

In some embodiments, the provided engineered islets comprise a modification, such as a genetic modification, of one or more target polynucleotide sequence that regulate one or more MHC class II molecules. Exemplary methods for reducing expression of one or more MHC class II molecules are described in sections below. In some embodiments, the engineered islets comprise a genetic editing modification to the CIITA gene.

In some embodiments, the provided engineered islets comprise a modification, such as a genetic modification, of one or more target polynucleotide sequence that regulate one or more MHC class I molecules and one or more MHC class II molecules. Exemplary methods for reducing expression of one or more MHC class I molecules and one or more MHC class II molecules are described in sections below. In some embodiments, the engineered islets comprise genetic editing modifications to the B2M and NLRC5 genes. In some embodiments, the engineered islets comprise genetic editing modifications to the CIITA and NLRC5 genes. In particular embodiments, the cell comprises genetic editing modifications to the B2M, CIITA and NLRC5 genes.

In some embodiments, the modification that reduces B2M, CIITA and/or NLRC5 expression reduces B2M, CIITA and/or NLRC5 mRNA expression. In some embodiments, the reduced mRNA expression of B2M, CIITA and/or NLRC5 is relative to an unmodified or wild-type cell of the same cell type that does not comprise the modification. In some embodiments, the mRNA expression of B2M is reduced by more than about 5%, such as reduced by more than about any of 10%, 20%, 30%, 40%, 50%, 60%, 70%, 80%, 90%, or more. In some embodiments, the mRNA expression of B2M, CIITA and/or NLRC5 is reduced by up to about 100%, such as reduced by up to about any of 90%, 80%, 70%, 60%, 50%, 40%, 30%, 20%, 10%, 5%, or less. In some embodiments, the mRNA expression of B2M, CIITA and/or NLRC5 is reduced by any of about 5%, 10%, 20%, 30%, 40%, 50%, 60%, 70%, 80%, 90%, or 100%. In some embodiments, the mRNA expression of B2M, CIITA and/or NLRC5 is eliminated (e.g., 0% expression of B2M, CIITA and/or NLRC5 mRNA). In some embodiments, the modification that reduces B2M, CIITA and/or NLRC5 mRNA expression eliminates B2M, CIITA and/or NLRC5 gene activity.

In some embodiments, the modification that reduces B2M, CIITA and/or NLRC5 expression reduces B2M, CIITA and/or NLRC5 protein expression. In some embodiments, the reduced protein expression of B2M, CIITA and/or NLRC5 is relative to an unmodified or wild-type cell of the same cell type that does not comprise the modification. In some embodiments, the protein expression of B2M, CIITA and/or NLRC5 is reduced by more than about 5%, such as reduced by more than about any of 10%, 20%, 30%, 40%, 50%, 60%, 70%, 80%, 90%, or more. In some embodiments, the protein expression of B2M, CIITA and/or NLRC5 is reduced by up to about 100%, such as reduced by up to about any of 90%, 80%, 70%, 60%, 50%, 40%, 30%, 20%, 10%, 5%, or less. In some embodiments, the protein expression of B2M, CIITA and/or NLRC5 is reduced by any of about 5%, 10%, 20%, 30%, 40%, 50%, 60%, 70%, 80%, 90%, or 100%. In some embodiments, the protein expression of B2M, CIITA and/or NLRC5 is eliminated (e.g., 0% expression of B2M, CIITA and/or NLRC5 protein). In some embodiments, the modification that reduces B2M, CIITA and/or NLRC5 protein expression eliminates B2M, CIITA and/or NLRC5 gene activity.

In some embodiments, the modification that reduces B2M, CIITA and/or NLRC5 expression comprises inactivation or disruption of the B2M, CIITA and/or NLRC5 gene. In some embodiments, the modification that reduces B2M, CIITA and/or NLRC5 expression comprises inactivation or disruption of one allele of the B2M, CIITA and/or NLRC5 gene. In some embodiments, the modification that reduces B2M, CIITA and/or NLRC5 expression comprises inactivation or disruption comprises inactivation or disruption of both alleles of the B2M, CIITA and/or NLRC5 gene.

In some embodiments, the modification comprises inactivation or disruption of one or more B2M, CIITA and/or NLRC5 coding sequences in the cell. In some embodiments, the modification comprises inactivation or disruption of all B2M, CIITA and/or NLRC5 coding sequences in the cell. In some embodiments, the modification comprises inactivation or disruption comprises an indel in the B2M, CIITA and/or NLRC5 gene. In some embodiments, the modification is a frameshift mutation of genomic DNA of the B2M, CIITA and/or NLRC5 gene. In some embodiments, the modification is a deletion of genomic DNA of the B2M, CIITA and/or NLRC5 gene. In some embodiments, the modification is a deletion of a contiguous stretch of genomic DNA of the B2M, CIITA and/or NLRC5 gene. In some embodiments, the B2M, CIITA and/or NLRC5 gene is knocked out.

In some embodiments, the engineered islets comprise reduced expression of one or more MHC class I, or a component thereof, wherein reduced is as described herein, such as relative to prior to engineering to reduce expression of one or more MHC class I molecules or a component thereof, a reference cell or a reference cell population (such as a cell having a desired lack of an immunogenic response), or a measured value. In some embodiments, the engineered islets is modified to reduce cell surface expression of the one or more MHC class I polypeptides, or a component thereof (such as B2M). In some embodiments, cell surface expression of the one or more MHC class I polypeptides, or a component thereof (such as B2M), on the engineered islets is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of the one or more MHC class I polypeptides, or a component thereof (such as B2M), cell surface expression prior to being modified to reduce cell surface presentation of the one or more MHC class I polypeptides, or a component thereof (such as B2M). In some embodiments, cell surface expression of the one or more MHC class I polypeptides, or a component thereof (such as B2M), on the modified cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of the one or more MHC class I polypeptides, or a component thereof (such as B2M), cell surface expression on a reference cell or a reference cell population (such as an average amount of one or more MHC class I polypeptides, or a component thereof (such as B2M), cell surface expression). In some embodiments, there is no cell surface presentation of the one or more MHC class I polypeptides, or a component thereof (such as B2M), on the modified beta bell (including no detectable cell surface expression, including as measured using known techniques, e.g., flow cytometry). In some embodiments, the engineered islets exhibits reduced protein expression of the one or more MHC class I polypeptides, or a component thereof (such as B2M). In some embodiments, protein expression of the one or more MHC class I polypeptides, or a component thereof (such as B2M), of the modified cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of the one or more MHC class I polypeptides, or a component thereof (such as B2M), protein expression prior to being modified to reduce protein expression of the one or more MHC class I polypeptides, or a component thereof (such as B2M). In some embodiments, protein expression of the one or more MHC class I polypeptides, or a component thereof (such as B2M), of the engineered islets is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of the one or more MHC class I polypeptides, or a component thereof (such as B2M), prior to being modified to reduce protein expression of the one or more MHC class I polypeptides, or a component thereof (such as B2M). In some embodiments, the engineered islets exhibits no protein expression of the one or more MHC class I polypeptides, or a component thereof (such as B2M), (including no detectable protein expression, including as measured using known techniques, e.g., western blot or mass spectrometry). In some embodiments, the engineered islets does not comprise the one or more MHC class I polypeptides, or a component thereof (such as B2M) (including no detectable protein, including as measured using known techniques, e.g., western blot or mass spectrometry). In some embodiments, the engineered islets exhibits reduced mRNA expression encoding the one or more MHC class I polypeptides, or a component thereof (such as B2M). In some embodiments, mRNA expression encoding the one or more MHC class I polypeptides, or a component thereof (such as B2M), of the engineered islets is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of mRNA expression encoding the one or more MHC class I polypeptides, or a component thereof (such as B2M), prior to being modified to reduce mRNA expression of the one or more MHC polypeptides, or a component thereof (such as B2M). In some embodiments, mRNA expression encoding the one or more MHC class I polypeptides, or a component thereof (such as B2M), of the engineered islets is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of mRNA expression of a reference cell or a reference cell population. In some embodiments, the engineered islets does not express mRNA encoding one or more MHC class I polypeptides, or a component thereof (including no detectable mRNA expression, including as measured using known techniques, e.g., sequencing techniques or PCR). In some embodiments, the engineered islets does not comprise mRNA encoding one or more MHC class I polypeptides, or a component thereof (including no detectable mRNA, including as measured using known techniques, e.g., sequencing techniques or PCR). In some embodiments, the engineered islets comprise a gene inactivation or disruption of the one or more MHC class I molecules gene. In some embodiments, the engineered islets comprise a gene inactivation or disruption of the one or more MHC class I molecules gene in both alleles. In some embodiments, the engineered islets comprise a gene inactivation or disruption of the one or more MHC class I molecules gene in all alleles. In some embodiments, the engineered islets is a one or more MHC class I molecules knockout or a one or more MHC class I molecules component (such as B2M) knockout.

In some embodiments, the engineered islets comprise reduced expression of one or more MHC class II molecules, wherein reduced is as described herein, such as relative to prior to engineering to reduce one or more MHC class II molecules expression, a reference cell or a reference cell population (such as a cell having a desired lack of an immunogenic response), or a measured value. In some embodiments, the engineered islets is engineered to reduced cell surface expression of the one or more MHC class II polypeptides. In some embodiments, cell surface expression of the one or more MHC class II polypeptides on the engineered islets is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of the one or more MHC class II polypeptides cell surface expression prior to being modified to reduce cell surface presentation of the one or more MHC class II polypeptides. In some embodiments, cell surface expression of the one or more MHC class II polypeptides on the engineered islets is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of the one or more MHC class II polypeptides cell surface expression on a reference cell or a reference cell population (such as an average amount of one or more MHC class II polypeptides cell surface expression). In some embodiments, there is no cell surface presentation of the one or more MHC class II polypeptides on the engineered islets (including no detectable cell surface expression, including as measured using known techniques, e.g., flow cytometry). In some embodiments, the engineered islets exhibits reduced protein expression of the one or more MHC class II polypeptides. In some embodiments, protein expression of the one or more MHC class II polypeptides of the engineered islets is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of the one or more MHC class II polypeptides protein expression prior to being modified beta to reduce protein expression of the one or more MHC class II polypeptides. In some embodiments, protein expression of the MHC class II polypeptides of the engineered islets is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of the one or more MHC class II polypeptides prior to being modified to reduce protein expression of the one or more MHC class II polypeptides. In some embodiments, the engineered islets exhibits no protein expression of the one or more MHC class II polypeptides (including no detectable protein expression, including as measured using known techniques, e.g., western blot or mass spectrometry). In some embodiments, the engineered islets does not comprise the one or more MHC class II polypeptides (including no detectable protein, including as measured using known techniques, e.g., western blot or mass spectrometry). In some embodiments, the engineered islets exhibits reduced mRNA expression encoding the one or more MHC class II polypeptides. In some embodiments, mRNA expression encoding the one or more MHC class II polypeptides of the engineered islets is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of mRNA expression encoding the one or more MHC class II polypeptides prior to being modified beta to reduce mRNA expression of the one or more MHC class II polypeptides. In some embodiments, mRNA expression encoding the one or more MHC class II polypeptides of the engineered islets is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of mRNA expression of a reference cell or a reference cell population. In some embodiments, the engineered islets does not express mRNA encoding one or more MHC class II polypeptides (including no detectable mRNA expression, including as measured using known techniques, e.g., sequencing techniques or PCR). In some embodiments, the engineered islets does not comprise mRNA encoding one or more MHC class II polypeptides (including no detectable mRNA, including as measured using known techniques, e.g., sequencing techniques or PCR). In some embodiments, the engineered islets comprise a gene inactivation or disruption of the one or more MHC class II molecules gene. In some embodiments, the engineered islets comprise a gene inactivation or disruption of the one or more MHC class II molecules gene in both alleles. In some embodiments, the engineered islets comprise a gene inactivation or disruption of the one or more MHC class II molecules in all alleles. In some embodiments, the engineered islets is a one or more MHC class II molecules knockout.

### 1. Methods of Reducing Expression

In some embodiments, the cells provided herein are modified, such as genetically modified, to reduce expression of the one or more target polynucleotides as described. In some embodiments, the cell that is engineered (e.g., modified) with the one or more modifications to reduce (e.g. eliminate) expression of a polynucleotide or protein is any source cell as described herein. In some embodiments, the source cell is any cell described herein. In certain embodiments, the cells (e.g., beta cells) disclosed herein comprise one or more modifications, such as genetic modifications, to reduce expression of one or more target polynucleotides. Non-limiting examples of the one or more target polynucleotides include any as described above, such as one or more of MHC class I molecules, or a component thereof, one or more MHC class II molecules, CIITA, B2M, NLRC5, HLA-A, HLA-B, HLA-C, LRC5, RFX-ANK, RFX5, RFX-AP, NFY-A, NFY-B, NFY-C, IRF1, and TAP1. In some embodiments, the one or more modifications, such as genetic modifications, to reduce expression of the one or more target polynucleotides is combined with one or more modifications to increase expression of a desired transgene, such as any described herein. In some embodiments, the one or more modifications, such as genetic modifications, create engineered islets that are immune-privileged or hypoimmunogenic cells. By modulating (e.g., reducing or deleting) expression of one or a plurality of the target polynucleotides, such cells exhibit decreased immune activation when engrafted into a recipient subject. In some embodiments, the cell is considered hypoimmunogenic, e.g., in a recipient subject or patient upon administration.

Any method for reducing expression of a target polynucleotide may be used. In some embodiments, the modifications (e.g., genetic modifications) result in permanent elimination or reduction in expression of the target polynucleotide. For instance, in some embodiments, the target polynucleotide or gene is disrupted by introducing a DNA break in the target polynucleotide, such as by using a targeting endonuclease. In other embodiments, the modifications (e.g., genetic modifications) result in transient reduction in expression of the target polynucleotide. For instance, in some embodiments gene repression is achieved using an inhibitory nucleic acid that is complementary to the target polynucleotide to selectively suppress or repress expression of the gene, for instance using antisense techniques, such as by RNA interference (RNAi), short interfering RNA (siRNA), short hairpin (shRNA), and/or ribozymes.

In some embodiments, the target polynucleotide sequence is a genomic sequence. In some embodiments, the target polynucleotide sequence is a human genomic sequence. In some embodiments, the target polynucleotide sequence is a mammalian genomic sequence. In some embodiments, the target polynucleotide sequence is a vertebrate genomic sequence.

In some embodiments, any of gene editing technologies can be used to reduce expression of the one or more target polynucleotides or target proteins as described. In some embodiments, the gene editing technology can include systems involving nucleases, integrases, transposases, recombinases. In some embodiments, the gene editing technologies can be used for knock-out or knock-down of genes. In some embodiments, the gene-editing technologies can be used for knock-in or integration of DNA into a region of the genome. In some embodiments, the gene editing technology mediates single-strand breaks (SSB). In some embodiments, the gene editing technology mediates double-strand breaks (DSB), including in connection with non-homologous end-joining (NHEJ) or homology-directed repair (HDR). In some embodiments, the gene editing technology can include DNA-based editing or prime-editing. In some embodiments, the gene editing technology can include Programmable Addition via Site-specific Targeting Elements (PASTE).

In some embodiments, gene disruption is carried out by induction of one or more double-stranded breaks and/or one or more single-stranded breaks in the gene, typically in a targeted manner. In some embodiments, the double-stranded or single-stranded breaks are made by a nuclease, e.g., an endonuclease, such as a gene-targeted nuclease. In some embodiments, the targeted nuclease is selected from zinc finger nucleases (ZFN), transcription activator-like effector nucleases (TALENs), and RNA-guided nucleases such as a CRISPR-associated nuclease (Cas), specifically designed to be targeted to the sequence of a gene or a portion thereof. In some embodiments, the targeted nuclease generates double-stranded or single-stranded breaks that then undergo repair through error prone non-homologous end joining (NHEJ) or, in some cases, precise homology directed repair (HDR) in which a template is used. In some embodiments, the targeted nuclease generates DNA double strand breaks (DSBs). In some embodiments, the process of producing and repairing the breaks is typically error prone and results in insertions and deletions (indels) of DNA bases from NHEJ repair. In some embodiments, the genetic modification may induce a deletion, insertion or mutation of the nucleotide sequence of the target gene. In some cases, the genetic modification may result in a frameshift mutation, which can result in a premature stop codon. In examples of nuclease-mediated gene editing the targeted edits occur on both alleles of the gene resulting in a biallelic disruption or edit of the gene. In some embodiments, all alleles of the gene are targeted by the gene editing. In some embodiments, genetic modification with a targeted nuclease, such as using a CRISPR/Cas system, leads to complete knockout of the gene.

In some embodiments, the nuclease, such as a rare-cutting endonuclease, is introduced into a cell containing the target polynucleotide sequence. The nuclease may be introduced into the cell in the form of a nucleic acid encoding the nuclease. The process of introducing the nucleic acids into cells can be achieved by any suitable technique. Suitable techniques include calcium phosphate or lipid-mediated transfection, electroporation, and transduction or infection using a viral vector. In some embodiments, the nucleic acid that is introduced into the cell is DNA. In some embodiments, the nuclease is introduced into the cell in the form of a protein. For instance, in the case of a CRISPR/Cas system a ribonucleoprotein (RNP) may be introduced into the cell.

In some embodiments, the modification (e.g., genetic modification) occurs using a CRISPR/Cas system. Any CRISPR/Cas system that is capable of altering a target polynucleotide sequence in a cell can be used. Such CRISPR-Cas systems can employ a variety of Cas proteins (Haft et al. PloS Comput Biol. 2005; 1(6)e60). The molecular machinery of such Cas proteins that allows the CRISPR/Cas system to alter target polynucleotide sequences in cells include RNA binding proteins, endo- and exo-nucleases, helicases, and polymerases. In some embodiments, the CRISPR/Cas system is a CRISPR type I system. In some embodiments, the CRISPR/Cas system is a CRISPR type II system. In some embodiments, the CRISPR/Cas system is a CRISPR type V system.

The CRISPR/Cas systems includes targeted systems that can be used to alter any target polynucleotide sequence in a cell. In some embodiments, a CRISPR/Cas system provided herein includes a Cas protein and one or more, such as at least one to two, ribonucleic acids (e.g., guide RNA (gRNA)) that are capable of directing the Cas protein to and hybridizing to a target motif of a target polynucleotide sequence.

In some embodiments, a Cas protein comprises one or more amino acid substitutions or modifications. In some embodiments, the one or more amino acid substitutions comprises a conservative amino acid substitution. In some instances, substitutions and/or modifications can prevent or reduce proteolytic degradation and/or extend the half-life of the polypeptide in a cell. In some embodiments, the Cas protein can comprise a peptide bond replacement (e.g., urea, thiourea, carbamate, sulfonyl urea, etc.). In some embodiments, the Cas protein can comprise a naturally occurring amino acid. In some embodiments, the Cas protein can comprise an alternative amino acid (e.g., D-amino acids, beta-amino acids, homocysteine, phosphoserine, etc.). In some embodiments, a Cas protein can comprise a modification to include a moiety (e.g., PEGylation, glycosylation, lipidation, acetylation, end-capping, etc.).

In some embodiments, a Cas protein comprises a core Cas protein. Exemplary Cas core proteins include, but are not limited to, Cas1, Cas2, Cas3, Cas4, Cas5, Cas6, Cas7, Cas8, Cas9, Cas12a, and Cas13. In some embodiments, a Cas protein comprises a Cas protein of an E. coli subtype (also known as CASS2). Exemplary Cas proteins of the E. Coli subtype include, but are not limited to Cse1, Cse2, Cse3, Cse4, and Cas5e. In some embodiments, a Cas protein comprises a Cas protein of the Ypest subtype (also known as CASS3). Exemplary Cas proteins of the Ypest subtype include, but are not limited to Csy1, Csy2, Csy3, and Csy4. In some embodiments, a Cas protein comprises a Cas protein of the Nmeni subtype (also known as CASS4). Exemplary Cas proteins of the Nmeni subtype include, but are not limited to Csn1 and Csn2. In some embodiments, a Cas protein comprises a Cas protein of the Dvulg subtype (also known as CASS1). Exemplary Cas proteins of the Dvulg subtype include Csd1, Csd2, and Cas5d. In some embodiments, a Cas protein comprises a Cas protein of the Tneap subtype (also known as CASS7). Exemplary Cas proteins of the Tneap subtype include, but are not limited to, Cst1, Cst2, Cas5t. In some embodiments, a Cas protein comprises a Cas protein of the Hmari subtype. Exemplary Cas proteins of the Hmari subtype include, but are not limited to Csh1, Csh2, and Cas5 h. In some embodiments, a Cas protein comprises a Cas protein of the Apern subtype (also known as CASS5). Exemplary Cas proteins of the Apern subtype include, but are not limited to Csa1, Csa2, Csa3, Csa4, Csa5, and Cas5a. In some embodiments, a Cas protein comprises a Cas protein of the Mtube subtype (also known as CASS6). Exemplary Cas proteins of the Mtube subtype include, but are not limited to Csm1, Csm2, Csm3, Csm4, and Csm5. In some embodiments, a Cas protein comprises a RAMP module Cas protein. Exemplary RAMP module Cas proteins include, but are not limited to, Cmr1, Cmr2, Cmr3, Cmr4, Cmr5, and Cmr6. See, e.g., Klompe et al., Nature 571, 219-225 (2019); Strecker et al., Science 365, 48-53 (2019).

In some embodiments, the methods for genetically modifying cells to knock out, knock down, or otherwise modify one or more genes comprise using a site-directed nuclease, including, for example, zinc finger nucleases (ZFNs), transcription activator-like effector nucleases (TALENs), meganucleases, transposases, and clustered regularly interspaced short palindromic repeat (CRISPR)/Cas systems

ZFNs are fusion proteins comprising an array of site-specific DNA binding domains adapted from zinc finger-containing transcription factors attached to the endonuclease domain of the bacterial FokI restriction enzyme. A ZFN may have one or more (e.g., 1, 2, 3, 4, 5, 6, 7, 8, 9, 10, or more) of the DNA binding domains or zinc finger domains. See, e.g., Carroll et al., Genetics Society of America (2011) 188:773-782; Kim et al., Proc. Natl. Acad. Sci. USA (1996) 93:1156-1160. Each zinc finger domain is a small protein structural motif stabilized by one or more zinc ions and usually recognizes a 3-to 4-bp DNA sequence. Tandem domains can thus potentially bind to an extended nucleotide sequence that is unique within a cell's genome.

Various zinc fingers of known specificity can be combined to produce multi-finger polypeptides which recognize about 6, 9, 12, 15, or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells. Zinc fingers can be engineered to bind a predetermined nucleic acid sequence. Criteria to engineer a zinc finger to bind to a predetermined nucleic acid sequence are known in the art. See, e.g., Sera et al., Biochemistry (2002) 41:7074-7081; Liu et al., Bioinformatics (2008) 24:1850-1857.

ZFNs containing FokI nuclease domains or other dimeric nuclease domains function as a dimer. Thus, a pair of ZFNs are required to target non-palindromic DNA sites. The two individual ZFNs must bind opposite strands of the DNA with their nucleases properly spaced apart. See Bitinaite et al., Proc. Natl. Acad. Sci. USA (1998) 95:10570-10575. To cleave a specific site in the genome, a pair of ZFNs are designed to recognize two sequences flanking the site, one on the forward strand and the other on the reverse strand. Upon binding of the ZFNs on either side of the site, the nuclease domains dimerize and cleave the DNA at the site, generating a DSB with 5′ overhangs. HDR can then be utilized to introduce a specific mutation, with the help of a repair template containing the desired mutation flanked by homology arms. The repair template is usually an exogenous double-stranded DNA vector introduced to the cell. See Miller et al., Nat. Biotechnol. (2011) 29:143-148; Hockemeyer et al., Nat. Biotechnol. (2011) 29:731-734.

TALENs are another example of an artificial nuclease which can be used to edit a target gene. TALENs are derived from DNA binding domains termed TALE repeats, which usually comprise tandem arrays with 10 to 30 repeats that bind and recognize extended DNA sequences. Each repeat is 33 to 35 amino acids in length, with two adjacent amino acids (termed the repeat-variable di-residue, or RVD) conferring specificity for one of the four DNA base pairs. Thus, there is a one-to-one correspondence between the repeats and the base pairs in the target DNA sequences.

TALENs are produced artificially by fusing one or more TALE DNA binding domains (e.g., 1, 2, 3, 4, 5, 6, 7, 8, 9, 10 or more) to a nuclease domain, for example, a FokI endonuclease domain. See Zhang, Nature Biotech. (2011) 29:149-153. Several mutations to FokI have been made for its use in TALENs; these, for example, improve cleavage specificity or activity. See Cermak et al., Nucl. Acids Res. (2011) 39:e82; Miller et al., Nature Biotech. (2011) 29:143-148; Hockemeyer et al., Nature Biotech. (2011) 29:731-734; Wood et al., Science (2011) 333:307; Doyon et al., Nature Methods (2010) 8:74-79; Szczepek et al., Nature Biotech (2007) 25:786-793; Guo et al., J. Mol. Biol. (2010) 200:96. The FokI domain functions as a dimer, requiring two constructs with unique DNA binding domains for sites in the target genome with proper orientation and spacing. Both the number of amino acid residues between the TALE DNA binding domain and the FokI nuclease domain and the number of bases between the two individual TALEN binding sites appear to be important parameters for achieving high levels of activity. Miller et al., Nature Biotech. (2011) 29:143-148.

By combining engineered TALE repeats with a nuclease domain, a site-specific nuclease can be produced specific to any desired DNA sequence. Similar to ZFNs, TALENs can be introduced into a cell to generate DSBs at a desired target site in the genome, and so can be used to knock out genes or knock in mutations in similar, HDR-mediated pathways. See Boch, Nature Biotech. (2011) 29:135-136; Boch et al., Science (2009) 326:1509-1512; Moscou et al., Science (2009) 326:3501.

Meganucleases are enzymes in the endonuclease family which are characterized by their capacity to recognize and cut large DNA sequences (from 14 to 40 base pairs). Meganucleases are grouped into families based on their structural motifs which affect nuclease activity and/or DNA recognition. The most widespread and best known meganucleases are the proteins in the LAGLIDADG family, which owe their name to a conserved amino acid sequence. See Chevalier et al., Nucleic Acids Res. (2001) 29(18): 3757-3774. On the other hand, the GIY-YIG family members have a GIY-YIG module, which is 70-100 residues long and includes four or five conserved sequence motifs with four invariant residues, two of which are required for activity. See Van Roey et al., Nature Struct. Biol. (2002) 9:806-811. The His-Cys family meganucleases are characterized by a highly conserved series of histidines and cysteines over a region encompassing several hundred amino acid residues. See Chevalier et al., Nucleic Acids Res. (2001) 29(18):3757-3774. Members of the NHN family are defined by motifs containing two pairs of conserved histidines surrounded by asparagine residues. See Chevalier et al., Nucleic Acids Res. (2001) 29(18):3757-3774.

Because the chance of identifying a natural meganuclease for a particular target DNA sequence is low due to the high specificity requirement, various methods including mutagenesis and high throughput screening methods have been used to create meganuclease variants that recognize unique sequences. Strategies for engineering a meganuclease with altered DNA-binding specificity, e.g., to bind to a predetermined nucleic acid sequence are known in the art. See, e.g., Chevalier et al., Mol. Cell. (2002) 10:895-905; Epinat et al., Nucleic Acids Res (2003) 31:2952-2962; Silva et al., J Mol. Biol. (2006) 361:744-754; Seligman et al., Nucleic Acids Res (2002) 30:3870-3879; Sussman et al., J Mol Biol (2004) 342:31-41; Doyon et al., J Am Chem Soc (2006) 128:2477-2484; Chen et al., Protein Eng Des Sel (2009) 22:249-256; Arnould et al., J Mol Biol. (2006) 355:443-458; Smith et al., Nucleic Acids Res. (2006) 363(2):283-294.

Like ZFNs and TALENs, Meganucleases can create DSBs in the genomic DNA, which can create a frame-shift mutation if improperly repaired, e.g., via NHEJ, leading to a decrease in the expression of a target gene in a cell. Alternatively, foreign DNA can be introduced into the cell along with the meganuclease. Depending on the sequences of the foreign DNA and chromosomal sequence, this process can be used to modify the target gene. See Silva et al., Current Gene Therapy (2011) 11:11-27.

Transposases are enzymes that bind to the end of a transposon and catalyze its movement to another part of the genome by a cut and paste mechanism or a replicative transposition mechanism. By linking transposases to other systems such as the CRISPER/Cas system, new gene editing tools can be developed to enable site specific insertions or manipulations of the genomic DNA. There are two known DNA integration methods using transposons which use a catalytically inactive Cas effector protein and Tn7-like transposons. The transposase-dependent DNA integration does not provoke DSBs in the genome, which may guarantee safer and more specific DNA integration.

The CRISPR system was originally discovered in prokaryotic organisms (e.g., bacteria and archaea) as a system involved in defense against invading phages and plasmids that provides a form of acquired immunity. Now it has been adapted and used as a popular gene editing tool in research and clinical applications.

CRISPR/Cas systems generally comprise at least two components: one or more guide RNAs (gRNAs) and a Cas protein. The Cas protein is a nuclease that introduces a DSB into the target site. CRISPR-Cas systems fall into two major classes: class 1 systems use a complex of multiple Cas proteins to degrade nucleic acids; class 2 systems use a single large Cas protein for the same purpose. Class 1 is divided into types I, III, and IV; class 2 is divided into types II, V, and VI. Different Cas proteins adapted for gene editing applications include, but are not limited to, Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12 h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7. The most widely used Cas9 is a type II Cas protein and is described herein as illustrative. These Cas proteins may be originated from different source species. For example, Cas9 can be derived from S. pyogenes or S. aureus.

In the original microbial genome, the type II CRISPR system incorporates sequences from invading DNA between CRISPR repeat sequences encoded as arrays within the host genome. Transcripts from the CRISPR repeat arrays are processed into CRISPR RNAs (crRNAs) each harboring a variable sequence transcribed from the invading DNA, known as the “protospacer” sequence, as well as part of the CRISPR repeat. Each crRNA hybridizes with a second transactivating CRISPR RNA (tracrRNA), and these two RNAs form a complex with the Cas9 nuclease. The protospacer-encoded portion of the crRNA directs the Cas9 complex to cleave complementary target DNA sequences, provided that they are adjacent to short sequences known as “protospacer adjacent motifs” (PAMs).

Since its discovery, the CRISPR system has been adapted for inducing sequence specific DSBs and targeted genome editing in a wide range of cells and organisms spanning from bacteria to eukaryotic cells including human cells. In its use in gene editing applications, artificially designed, synthetic gRNAs have replaced the original crRNA:tracrRNA complex. For example, the gRNAs can be single guide RNAs (sgRNAs) composed of a crRNA, a tetraloop, and a tracrRNA. The crRNA usually comprises a complementary region (also called a spacer, usually about 20 nucleotides in length) that is user-designed to recognize a target DNA of interest. The tracrRNA sequence comprises a scaffold region for Cas nuclease binding. The crRNA sequence and the tracrRNA sequence are linked by the tetraloop and each have a short repeat sequence for hybridization with each other, thus generating a chimeric sgRNA. One can change the genomic target of the Cas nuclease by simply changing the spacer or complementary region sequence present in the gRNA. The complementary region will direct the Cas nuclease to the target DNA site through standard RNA-DNA complementary base pairing rules.

In order for the Cas nuclease to function, there must be a PAM immediately downstream of the target sequence in the genomic DNA. Recognition of the PAM by the Cas protein is thought to destabilize the adjacent genomic sequence, allowing interrogation of the sequence by the gRNA and resulting in gRNA-DNA pairing when a matching sequence is present. The specific sequence of PAM vanes depending on the species of the Cas gene. For example, the most commonly used Cas9 nuclease derived from S. pyogenes recognizes a PAM sequence of 5′-NGG-3′ or, at less efficient rates, 5′-NAG-3′, where “N” can be any nucleotide. Other Cas nuclease variants with alternative PAMs have also been characterized and successfully used for genome editing, which are summarized in Table 1a below.

TABLE 1a
Exemplary Cas nuclease variants and their PAM sequences

| CRISPR Nuclease | Source Organism | PAM Sequence (5′→3′) |
|---|---|---|
| SpCas9 | Streptococcus pyogenes | NGG or NAG |
| SaCas9 | Staphylococcus aureus | NGRRT or NGRRN |
| NmeCas9 | Neisseria meningitidis | NNNNGATT |
| CjCas9 | Campylobacter jejuni | NNNNRYAC |
| StCas9 | Streptococcus thermophilus | NNAGAAW |
| TdCas9 | Treponema denticola | NAAAAC |
| LbCas12a (Cpf1) | Lachnospiraceae bacterium | TTTV |
| AsCas 12a (Cpf1) | Acidaminococcus sp. | TTTV |
| AacCas12b | Alicyclobacillus acidiphilus | TTN |
| BhCas 12b v4 | Bacillus hisashii | ATTN, TTTN, or GTTN |

R = A or G; Y = C or T; W = A or T; V = A or C or G; N = any base

In some embodiments, Cas nucleases may comprise one or more mutations to alter their activity, specificity, recognition, and/or other characteristics. For example, the Cas nuclease may have one or more mutations that alter its fidelity to mitigate off-target effects (e.g., eSpCas9, SpCas9-HF1, HypaSpCas9, HeFSpCas9, and evoSpCas9 high-fidelity variants of SpCas9). For another example the Cas nuclease may have one or more mutations that alter its PAM specificity.

In some embodiments, a Cas protein comprises any one of the Cas proteins described herein or a functional portion thereof. As used herein, “functional portion” refers to a portion of a peptide which retains its ability to complex with at least one ribonucleic acid (e.g., guide RNA (gRNA)) and cleave a target polynucleotide sequence. In some embodiments, the functional portion comprises a combination of operably linked Cas9 protein functional domains selected from the group consisting of a DNA binding domain, at least one RNA binding domain, a helicase domain, and an endonuclease domain. In some embodiments, the functional portion comprises a combination of operably linked Cas12a (also known as Cpf1) protein functional domains selected from the group consisting of a DNA binding domain, at least one RNA binding domain, a helicase domain, and an endonuclease domain. In some embodiments, the functional domains form a complex. In some embodiments, a functional portion of the Cas9 protein comprises a functional portion of a RuvC-like domain. In some embodiments, a functional portion of the Cas9 protein comprises a functional portion of the HNH nuclease domain. In some embodiments, a functional portion of the Cas12a protein comprises a functional portion of a RuvC-like domain.

In some embodiments, suitable Cas proteins include, but are not limited to, Cas0, Cas12a (i.e. Cpf1), Cas12b, Cas12i, CasX, and Mad7.

In some embodiments, exogenous Cas protein can be introduced into the cell in polypeptide form. In certain embodiments, Cas proteins can be conjugated to or fused to a cell-penetrating polypeptide or cell-penetrating peptide. As used herein, “cell-penetrating polypeptide” and “cell-penetrating peptide” refers to a polypeptide or peptide, respectively, which facilitates the uptake of molecule into a cell. The cell-penetrating polypeptides can contain a detectable label.

In certain embodiments, Cas proteins can be conjugated to or fused to a charged protein (e.g., that carries a positive, negative or overall neutral electric charge). Such linkage may be covalent. In some embodiments, the Cas protein can be fused to a superpositively charged GFP to significantly increase the ability of the Cas protein to penetrate a cell (Cronican et al. ACS Chem Biol. 2010; 5(8):747-52). In certain embodiments, the Cas protein can be fused to a protein transduction domain (PTD) to facilitate its entry into a cell. Exemplary PTDs include Tat, oligoarginine, and penetratin. In some embodiments, the Cas9 protein comprises a Cas9 polypeptide fused to a cell-penetrating peptide. In some embodiments, the Cas9 protein comprises a Cas9 polypeptide fused to a PTD. In some embodiments, the Cas9 protein comprises a Cas9 polypeptide fused to a tat domain. In some embodiments, the Cas9 protein comprises a Cas9 polypeptide fused to an oligoarginine domain. In some embodiments, the Cas9 protein comprises a Cas9 polypeptide fused to a penetratin domain. In some embodiments, the Cas9 protein comprises a Cas9 polypeptide fused to a superpositively charged GFP. In some embodiments, the Cas12a protein comprises a Cas12a polypeptide fused to a cell-penetrating peptide. In some embodiments, the Cas12a protein comprises a Cas12a polypeptide fused to a PTD. In some embodiments, the Cas12a protein comprises a Cas12a polypeptide fused to a tat domain. In some embodiments, the Cas12a protein comprises a Cas12a polypeptide fused to an oligoarginine domain. In some embodiments, the cas12a protein comprises a Cas12a polypeptide fused to a penetratin domain. In some embodiments, the Cas12a protein comprises a Cas12a polypeptide fused to a superpositively charged GFP.

In some embodiments, the Cas protein can be introduced into a cell containing the target polynucleotide sequence in the form of a nucleic acid encoding the Cas protein. The process of introducing the nucleic acids into cells can be achieved by any suitable technique. Suitable techniques include calcium phosphate or lipid-mediated transfection, electroporation, and transduction or infection using a viral vector. In some embodiments, the nucleic acid comprises DNA. In some embodiments, the nucleic acid comprises a modified DNA, as described herein. In some embodiments, the nucleic acid comprises mRNA. In some embodiments, the nucleic acid comprises a modified mRNA, as described herein (e.g., a synthetic, modified mRNA).

In some embodiments, the Cas protein is complexed with one to two ribonucleic acids (e.g., guide RNA (gRNA)). In some embodiments, the Cas protein is complexed with two ribonucleic acids. In some embodiments, the Cas protein is complexed with one ribonucleic acid. In some embodiments, the Cas protein is encoded by a modified nucleic acid, as described herein (e.g., a synthetic, modified mRNA).

In provided embodiments, a CRISPR/Cas system generally includes two components: one or more guide RNA (gRNA) and a Cas protein. In some embodiments, the Cas protein is complexed with the one or more, such as one to two, ribonucleic acids (e.g., guide RNA (gRNA)). In some embodiments, the Cas protein is complexed with two ribonucleic acids. In some embodiments, the Cas protein is complexed with one ribonucleic acid. In some embodiments, the Cas protein is encoded by a modified nucleic acid, as described herein (e.g., a synthetic, modified mRNA).

In some embodiments, gRNAs are short synthetic RNAs composed of a scaffold sequence for Cas binding and a user-designed spacer or complementary portion designated crRNA. The cRNA is composed of a crRNA targeting sequence (herein after also called a gRNA targeting sequence; usually about 20 nucleotides in length) that defines the genomic target to be modified and a region of crRNA repeat (e.g. GUUUUAGAGCUA; SEQ ID NO: 19). One can change the genomic target of the Cas protein by simply changing the complementary portion sequence (e.g. gRNA targeting sequence) present in the gRNA. In some embodiments the scaffold sequence for Cas binding is made up of a tracrRNA sequence (e.g.

UAGCAAGUUAAAAUAAGGCUAGUCCGUUAUCAACUUGAAAAAGUGGCACCGAGUCGGUG CUUU; SEQ ID NO: 20) that hybridizes to the crRNA through its anti-repeat sequence. The complex between crRNA:tracrRNA recruits the Cas nuclease (e.g. Cas9) and cleaves upstream of a protospacer-adjacent motif (PAM). In order for the Cas protein to function, there must be a PAM immediately downstream of the target sequence in the genomic DNA. Recognition of the PAM by the Cas protein is thought to destabilize the adjacent genomic sequence, allowing interrogation of the sequence by the gRNA and resulting in gRNA-DNA pairing when a matching sequence is present. The specific sequence of PAM varies depending on the species of the Cas gene. For example, the most commonly used Cas9 nuclease, derived from S. pyogenes, recognizes a PAM sequence of NGG. Other Cas9 variants and other nucleases with alternative PAMs have also been characterized and successfully used for genome editing. Thus, the CRISPR/Cas system can be used to create targeted DSBs at specified genomic loci that are complementary to the gRNA designed for the target loci. The crRNA and tracrRNA can be linked together with a loop sequence (e.g. a tetraloop; GAAA, SEQ ID NO: 21) for generation of a gRNA that is a chimeric single guide RNA (sgRNA; Hsu et al. 2013). sgRNA can be generated for DNA-based expression or by chemical synthesis.

In some embodiments, the complementary portion sequences (e.g. gRNA targeting sequence) of the gRNA will vary depending on the target site of interest. In some embodiments, the gRNAs comprise complementary portions specific to a sequence of a gene set forth in Table 1b. In some embodiments, the genomic locus targeted by the gRNAs is located within 4000 bp, within 3500 bp, within 3000 bp, within 2500 bp, within 2000 bp, within 1500 bp, within 1000 bp, or within 500 bp of any of the loci as described.

The methods disclosed herein contemplate the use of any ribonucleic acid that is capable of directing a Cas protein to and hybridizing to a target motif of a target polynucleotide sequence. In some embodiments, at least one of the ribonucleic acids comprises tracrRNA. In some embodiments, at least one of the ribonucleic acids comprises CRISPR RNA (crRNA). In some embodiments, a single ribonucleic acid comprises a guide RNA that directs the Cas protein to and hybridizes to a target motif of the target polynucleotide sequence in a cell. In some embodiments, at least one of the ribonucleic acids comprises a guide RNA that directs the Cas protein to and hybridizes to a target motif of the target polynucleotide sequence in a cell. In some embodiments, both of the one to two ribonucleic acids comprise a guide RNA that directs the Cas protein to and hybridizes to a target motif of the target polynucleotide sequence in a cell. The ribonucleic acids provided herein can be selected to hybridize to a variety of different target motifs, depending on the particular CRISPR/Cas system employed, and the sequence of the target polynucleotide, as will be appreciated by those skilled in the art. The one to two ribonucleic acids can also be selected to minimize hybridization with nucleic acid sequences other than the target polynucleotide sequence. In some embodiments, the one to two ribonucleic acids hybridize to a target motif that contains at least two mismatches when compared with all other genomic nucleotide sequences in the cell. In some embodiments, the one to two ribonucleic acids hybridize to a target motif that contains at least one mismatch when compared with all other genomic nucleotide sequences in the cell. In some embodiments, the one to two ribonucleic acids are designed to hybridize to a target motif immediately adjacent to a deoxyribonucleic acid motif recognized by the Cas protein. In some embodiments, each of the one to two ribonucleic acids are designed to hybridize to target motifs immediately adjacent to deoxyribonucleic acid motifs recognized by the Cas protein which flank a mutant allele located between the target motifs. In some embodiments, each of the one to two ribonucleic acids comprises guide RNAs that directs the Cas protein to and hybridizes to a target motif of the target polynucleotide sequence in a cell.

In some embodiments, one or two ribonucleic acids (e.g., guide RNAs) are complementary to and/or hybridize to sequences on the same strand of a target polynucleotide sequence. In some embodiments, one or two ribonucleic acids (e.g., guide RNAs) are complementary to and/or hybridize to sequences on the opposite strands of a target polynucleotide sequence. In some embodiments, the one or two ribonucleic acids (e.g., guide RNAs) are not complementary to and/or do not hybridize to sequences on the opposite strands of a target polynucleotide sequence. In some embodiments, the one or two ribonucleic acids (e.g., guide RNAs) are complementary to and/or hybridize to overlapping target motifs of a target polynucleotide sequence. In some embodiments, the one or two ribonucleic acids (e.g., guide RNAs) are complementary to and/or hybridize to offset target motifs of a target polynucleotide sequence.

In some embodiments, nucleic acids encoding Cas protein and nucleic acids encoding the at least one to two ribonucleic acids are introduced into a cell via viral transduction (e.g., lentiviral transduction). In some embodiments, the Cas protein is complexed with 1-2 ribonucleic acids. In some embodiments, the Cas protein is complexed with two ribonucleic acids. In some embodiments, the Cas protein is complexed with one ribonucleic acid. In some embodiments, the Cas protein is encoded by a modified nucleic acid, as described herein (e.g., a synthetic, modified mRNA).

Exemplary gRNA targeting sequences useful for CRISPR/Cas-based targeting of genes described herein are provided in Table 1b. The sequences can be found in WO2016183041 filed May 9, 2016, the disclosure including the Tables, Appendices, and Sequence Listing is incorporated herein by reference in its entirety.

TABLE 1b
Exemplary gRNA targeting sequences useful for targeting genes

| Gene Name | SEQ ID NO: | WO2016183041 |
|---|---|---|
| HLA-A | SEQ ID NOS: 2-1418 | Table 8, Appendix 1 |
| HLA-B | SEQ ID NOS: 1419-3277 | Table 9, Appendix 2 |
| HLA-C | SEQ ID NOS: 3278-5183 | Table 10, Appendix 3 |
| RFX-ANK | SEQ ID NOS: 95636-102318 | Table 11, Appendix 4 |
| NFY-A | SEQ ID NOS: 102319-121796 | Table 13, Appendix 6 |
| RFX5 | SEQ ID NOS: 85645-90115 | Table 16, Appendix 9 |
| RFX-AP | SEQ ID NOS: 90116-95635 | Table 17, Appendix 10 |
| NFY-B | SEQ ID NOS: 121797-135112 | Table 20, Appendix 13 |
| NFY-C | SEQ ID NOS: 135113-176601 | Table 22, Appendix 15 |
| IRF1 | SEQ ID NOS: 176602-182813 | Table 23, Appendix 16 |
| TAP1 | SEQ ID NOS: 182814-188371 | Table 24, Appendix 17 |
| CIITA | SEQ ID NOS: 5184-36352 | Table 12, Appendix 5 |
| B2M | SEQ ID NOS: 81240-85644 | Table 15, Appendix 8 |
| NLRC5 | SEQ ID NOS: 36353-81239 | Table 14, Appendix 7 |
| CD47 | SEQ ID NOS: 200784-231885 | Table 29, Appendix 22 |
| HLA-E | SEQ ID NOS: 189859-193183 | Table 19, Appendix 12 |
| HLA-F | SEQ ID NOS: 688808-699754 | Table 45, Appendix 38 |
| HLA-G | SEQ ID NOS: 188372-189858 | Table 18, Appendix 11 |
| PD-L1 | SEQ ID NOS: 193184-200783 | Table 21, Appendix 14 |

In some embodiments, it is within the level of a skilled artisan to identify new loci and/or gRNA targeting sequences for use in methods of genetic disruption to reduce or eliminate expression of a gene as described. For example, for CRISPR/Cas systems, when an existing gRNA targeting sequence for a particular locus (e.g., within a target gene, e.g. set forth in Table 1b) is known, an “inch worming” approach can be used to identify additional loci for targeted insertion of transgenes by scanning the flanking regions on either side of the locus for PAM sequences, which usually occurs about every 100 base pairs (bp) across the genome. The PAM sequence will depend on the particular Cas nuclease used because different nucleases usually have different corresponding PAM sequences. The flanking regions on either side of the locus can be between about 500 to 4000 bp long, for example, about 500 bp, about 1000 bp, about 1500 bp, about 2000 bp, about 2500 bp, about 3000 bp, about 3500 bp, or about 4000 bp long. When a PAM sequence is identified within the search range, a new guide can be designed according to the sequence of that locus for use in genetic disruption methods. Although the CRISPR/Cas system is described as illustrative, any gene-editing approaches as described can be used in this method of identifying new loci, including those using ZFNs, TALENS, meganucleases and transposases.

Additional exemplary Cas9 guide RNA sequences useful for CRISPR/Cas-based targeting of genes described herein are provided in Table 2.

TABLE 2
Additional exemplary Cas9 guide RNA sequences
useful for targeting genes

| | | PAM | Target | | SEQ ID |
|---|---|---|---|---|---|
| Gene | guide sequence | | site | gRNA cut location | NO |
| ABO | UCUCUCCAUGUGCAGUAGGA | AGG | Exon 7 | chr9: 133,257,541 | 25 |
| FUT1 | CUGGAUGUCGGAGGAGUACG | CGG | Exon 4 | chr19: 48,750,822 | 26 |
| RHD | GUCUCCGGAAACUCGAGGUG | AGG | Exon 2 | chr1: 25,284,622 | 27 |
| F3 | ACAGUGUAGACUUGAUUGAC | GGG | Exon 2 | chr1: 94,540,281 | 28 |
| (CD142) | | | | | |
| B2M | CGUGAGUAAACCUGAAUCUU | TGG | Exon 2 | chr15: 44,715,434 | 29 |
| CIITA | GAUAUUGGCAUAAGCCUCCC | TGG | Exon 3 | chr16: 10,895,747 | 30 |
| TRAC | AGAGUCUCUCAGCUGGUACA | CGG | Exon 1 | chr14: 22,5547,533 | 31 |

In some embodiments, the cells described herein are made using Transcription Activator-Like Effector Nucleases (TALEN) methodologies. By a “TALE-nuclease” (TALEN) is intended a fusion protein consisting of a nucleic acid-binding domain typically derived from a Transcription Activator Like Effector (TALE) and one nuclease catalytic domain to cleave a nucleic acid target sequence. The catalytic domain is preferably a nuclease domain and more preferably a domain having endonuclease activity, like for instance I-TevI, ColE7, NucA and Fok-I. In a particular embodiment, the TALE domain can be fused to a meganuclease like for instance I-CreI and I-OnuI or functional variant thereof. In a more preferred embodiment, said nuclease is a monomeric TALE-Nuclease. A monomeric TALE-Nuclease is a TALE-Nuclease that does not require dimerization for specific recognition and cleavage, such as the fusions of engineered TAL repeats with the catalytic domain of I-TevI described in WO2012138927. Transcription Activator like Effector (TALE) are proteins from the bacterial species Xanthomonas comprise a plurality of repeated sequences, each repeat comprising di-residues in position 12 and 13 (RVD) that are specific to each nucleotide base of the nucleic acid targeted sequence. Binding domains with similar modular base-per-base nucleic acid binding properties (MBBBD) can also be derived from new modular proteins recently discovered by the applicant in a different bacterial species. The new modular proteins have the advantage of displaying more sequence variability than TAL repeats. Preferably, RVDs associated with recognition of the different nucleotides are HD for recognizing C, NG for recognizing T, NI for recognizing A, NN for recognizing G or A, NS for recognizing A, C, G or T, HG for recognizing T, IG for recognizing T, NK for recognizing G, HA for recognizing C, ND for recognizing C, HI for recognizing C, HN for recognizing G, NA for recognizing G, SN for recognizing G or A and YG for recognizing T, TL for recognizing A, VT for recognizing A or G and SW for recognizing A. In another embodiment, critical amino acids 12 and 13 can be mutated towards other amino acid residues in order to modulate their specificity towards nucleotides A, T, C and G and in particular to enhance this specificity. TALEN kits are sold commercially.

In some embodiments, the cells are manipulated using zinc finger nuclease (ZFN). A “zinc finger binding protein” is a protein or polypeptide that binds DNA, RNA and/or protein, preferably in a sequence-specific manner, as a result of stabilization of protein structure through coordination of a zinc ion. The term zinc finger binding protein is often abbreviated as zinc finger protein or ZFP. The individual DNA binding domains are typically referred to as “fingers.” A ZFP has least one finger, typically two fingers, three fingers, or six fingers. Each finger binds from two to four base pairs of DNA, typically three or four base pairs of DNA. A ZFP binds to a nucleic acid sequence called a target site or target segment. Each finger typically comprises an approximately 30 amino acid, zinc-chelating, DNA-binding subdomain. Studies have demonstrated that a single zinc finger of this class consists of an alpha helix containing the two invariant histidine residues coordinated with zinc along with the two cysteine residues of a single beta turn (see, e.g., Berg & Shi, Science 271:1081-1085 (1996)).

In some embodiments, the cells described herein are made using a homing endonuclease. Such homing endonucleases are well-known to the art (Stoddard 2005). Homing endonucleases recognize a DNA target sequence and generate a single- or double-strand break. Homing endonucleases are highly specific, recognizing DNA target sites ranging from 12 to 45 base pairs (bp) in length, usually ranging from 14 to 40 bp in length. The homing endonuclease may for example correspond to a LAGLIDADG endonuclease, to an HNH endonuclease, or to a GIY-YIG endonuclease. In some embodiments, the homing endonuclease can be an I-CreI variant.

In some embodiments, the cells described herein are made using a meganuclease. Meganucleases are by definition sequence-specific endonucleases recognizing large sequences (Chevalier, B. S. and B. L. Stoddard, Nucleic Acids Res., 2001, 29, 3757-3774). They can cleave unique sites in living cells, thereby enhancing gene targeting by 1000-fold or more in the vicinity of the cleavage site (Puchta et al., Nucleic Acids Res., 1993, 21, 5034-5040; Rouet et al., Mol. Cell. Biol., 1994, 14, 8096-8106; Choulika et al., Mol. Cell. Biol., 1995, 15, 1968-1973; Puchta et al., Proc. Natl. Acad. Sci. USA, 1996, 93, 5055-5060; Sargent et al., Mol. Cell. Biol., 1997, 17, 267-77; Donoho et al., Mol. Cell. Biol, 1998, 18, 4070-4078; Elliott et al., Mol. Cell. Biol., 1998, 18, 93-101; Cohen-Tannoudji et al., Mol. Cell. Biol., 1998, 18, 1444-1448).

In some embodiments, the gene editing technology is associated with base editing. Base editors (Bes) are typically fusions of a Cas (“CRISPR-associated”) domain and a nucleobase modification domain (e.g., a natural or evolved deaminase, such as a cytidine deaminase that include APOBEC1 (“apolipoprotein B mRNA editing enzyme, catalytic polypeptide 1”), CDA (“cytidine deaminase”), and AID (“activation-induced cytidine deaminase”)) domains. In some cases, base editors may also include proteins or domains that alter cellular DNA repair processes to increase the efficiency and/or stability of the resulting single-nucleotide change.

In some aspects, currently available base editors include cytidine base editors (e.g., BE4) that convert target C·G to T·A and adenine base editors (e.g., ABE7.10) that convert target A·T to G·C. In some aspects, Cas9-targeted deamination was first demonstrated in connection with a Base Editor (BE) system designed to induce base changes without introducing double-strand DNA breaks. Further Rat deaminase APOBEC1 (rAPOBEC1) fused to deactivated Cas9 (dCas9) was used to successfully convert cytidines to thymidines upstream of the PAM of the sgRNA. In some aspects, this first BE system was optimized by changing the dCas9 to a “nickase” Cas9 D10A, which nicks the strand opposite the deaminated cytidine. Without being bound by theory, this is expected to initiate long-patch base excision repair (BER), where the deaminated strand is preferentially used to template the repair to produce a U:A base pair, which is then converted to T:A during DNA replication.

In some embodiments, the base editor is a nucleobase editor containing a first DNA binding protein domain that is catalytically inactive, a domain having base editing activity, and a second DNA binding protein domain having nickase activity, where the DNA binding protein domains are expressed on a single fusion protein or are expressed separately (e.g., on separate expression vectors). In some embodiments, the base editor is a fusion protein comprising a domain having base editing activity (e.g., cytidine deaminase or adenosine deaminase), and two nucleic acid programmable DNA binding protein domains (napDNAbp), a first comprising nickase activity and a second napDNAbp that is catalytically inactive, wherein at least the two napDNAbp are joined by a linker. In some embodiments, the base editor is a fusion protein that comprises a DNA domain of a CRISPR-Cas (e.g., Cas9) having nickase activity (nCas; nCas9), a catalytically inactive domain of a CRISPR-Cas protein (e.g., Cas9) having nucleic acid programmable DNA binding activity (dCas; e.g., dCas9), and a deaminase domain, wherein the dCas is joined to the nCas by a linker, and the dCas is immediately adjacent to the deaminase domain. In some embodiments, the base editor is a adenine-to-thymine or “ATBE” (or thymine-to-adenine or “TABE”) transversion base editors. Exemplary base editor and base editor systems include any as described in patent publication Nos. US20220127622, US20210079366, US20200248169, US20210093667, US20210071163, WO2020181202, WO2021158921, WO2019126709, WO2020181178, WO2020181195, WO2020214842, WO2020181193, which are hereby incorporated in their entirety.

In some embodiments, the gene editing technology is target-primed reverse transcription (TPRT) or “prime editing”. In some embodiments, prime editing mediates targeted insertions, deletions, all 12 possible base-to-base conversions, and combinations thereof in human cells without requiring DSBs or donor DNA templates.

Prime editing is a genome editing method that directly writes new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“PEgRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution) shares the same sequence as the endogenous strand of the target site to be edited (with the exception that it includes the desired edit). Through DNA repair and/or replication machinery, the endogenous strand of the target site is replaced by the newly synthesized replacement strand containing the desired edit. In some cases, prime editing may be thought of as a “search-and-replace” genome editing technology since the prime editors search and locate the desired target site to be edited, and encode a replacement strand containing a desired edit which is installed in place of the corresponding target site endogenous DNA strand at the same time. For example, prime editing can be adapted for conducting precision CRISPR/Cas-based genome editing in order to bypass double stranded breaks. In some embodiments, the homologous protein is or encodes for a Cas protein-reverse transcriptase fusions or related systems to target a specific DNA sequence with a guide RNA, generate a single strand nick at the target site, and use the nicked DNA as a primer for reverse transcription of an engineered reverse transcriptase template that is integrated with the guide RNA. In some embodiments, the prime editor protein is paired with two prime editing guide RNAs (pegRNAs) that template the synthesis of complementary DNA flaps on opposing strands of genomic DNA, resulting in the replacement of endogenous DNA sequence between the PE-induced nick sites with pegRNA-encoded sequences.

In some embodiments, the gene editing technology is associated with a prime editor that is a reverse transcriptase, or any DNA polymerase known in the art. Thus, in one aspect, the prime editor may comprise Cas9 (or an equivalent napDNAbp) which is programmed to target a DNA sequence by associating it with a specialized guide RNA (i.e., PEgRNA) containing a spacer sequence that anneals to a complementary protospacer in the target DNA. Such methods include any disclosed in Anzalone et al., (doi.org/10.1038/s41586-019-1711-4), or in PCT publication Nos. WO2020191248, WO2021226558, or WO2022067130, which are hereby incorporated in their entirety.

In some embodiments, the gene editing technology is Programmable Addition via Site-specific Targeting Elements (PASTE). In some aspects, PASTE is platform in which genomic insertion is directed via a CRISPR-Cas9 nickase fused to both a reverse transcriptase and serine integrase. As described in Ioannidi et al. (doi.org/10.1101/2021.11.01.466786), PASTE does not generate double stranded breaks, but allows for integration of sequences as large as −36 kb. In some embodiments, the serine integrase can be any known in the art. In some embodiments, the serine integrase has sufficient orthogonality such that PASTE can be used for multiplexed gene integration, simultaneously integrating at least two different genes at least two genomic loci. In some embodiments, PASTE has editing efficiencies comparable to or better than those of homology directed repair or non-homologous end joining based integration, with activity in nondividing cells and fewer detectable off-target events.

In some embodiments, the cells provided herein are made using RNA silencing or RNA interference (RNAi) to knockdown (e.g., decrease, eliminate, or inhibit) the expression of a polypeptide. Useful RNAi methods include those that utilize synthetic RNAi molecules, short interfering RNAs (siRNAs), PIWI-interacting NRAs (piRNAs), short hairpin RNAs (shRNAs), microRNAs (miRNAs), and other transient knockdown methods recognized by those skilled in the art. Reagents for RNAi including sequence specific shRNAs, siRNA, miRNAs and the like are commercially available. For instance, a target polynucleotide, such as any described above, e.g. CIITA, B2M, or NLRC5, can be knocked down in a cell by RNA interference by introducing an inhibitory nucleic acid complementary to a target motif of the target polynucleotide, such as an siRNA, into the cells. In some embodiments, a target polynucleotide, such as any described above, e.g. CIITA, B2M, or NLRC5, can be knocked down in a cell by transducing a shRNA-expressing virus into the cell. In some embodiments, RNA interference is employed to reduce or inhibit the expression of at least one selected from the group consisting of CIITA, B2M, and NLRC5.

In some embodiments, the modification, such as the genetic modification, reduces or eliminates, such as knocks out, the expression of one or more MHC class I molecules genes and/or one or more MHC class II molecule genes by targeting the accessory chain B2M. B2M, TAP I, NLRC5, CIITA, HLA-A, HLA-B, HLA-C, HLA-DP, HLA-DM, HLA-DOA, HLA-DOB, HLA-DQ, HLA-DR, RFX5, RFXANK, RFXAP, NFY-A, NFY-B and/or NFY-C and any combination thereof. In some embodiments, decreased or eliminated expression of one or more MHC class I molecules and/or one or more MHC class II molecules is a modification that reduces expression of, e.g., knocks out, one or more of the following B2M. B2M, TAP I, NLRC5, CIITA, HLA-A, HLA-B, HLA-C, HLA-DP, HLA-DM, HLA-DOA, HLA-DOB, HLA-DQ, HLA-DR, RFX5, RFXANK, RFXAP, NFY-A, NFY-B and/or NFY-C.

### 2. Exemplary Target Polynucleotides and Methods for Reducing Expression

a. MHC Class I Molecules

In certain embodiments, the modification, such as the genetic modification, reduces or eliminates, such as knocks out, the expression of one or more MHC class I molecules genes by targeting the accessory chain B2M. In some embodiments, the genetic modification occurs using a CRISPR/Cas system. By reducing or eliminating, such as knocking out, expression of B2M, surface trafficking of one or more MHC class I molecules is blocked and such cells exhibit immune tolerance when engrafted into a recipient subject. In some embodiments, the cell is considered hypoimmunogenic, e.g., in a recipient subject or patient upon administration.

In some embodiments, the target polynucleotide sequence provided herein is a variant of B2M. In some embodiments, the target polynucleotide sequence is a homolog of B2M. In some embodiments, the target polynucleotide sequence is an ortholog of B2M.

In some embodiments, decreased or eliminated expression of one or more MHC class I molecules is a modification that reduces expression of one or more of the following MHC class I molecules—HLA-A, HLA-B, and HLA-C. In some embodiments, decreased or eliminated expression of B2M reduces or eliminates expression of one or more of the following MHC class I molecules—HLA-A, HLA-B, and HLA-C. In some embodiments, decreased or eliminated expression of B2M reduces or eliminates expression of an HLA-A protein. In some embodiments, decreased or eliminated expression of B2M reduces or eliminates expression of an HLA-B protein. In some embodiments, decreased or eliminated expression of B2M reduces or eliminates expression of an HLA-C protein. In some embodiments, decreased or eliminated expression of B2M reduces or eliminates expression of one or more of the following MHC class I molecules—HLA-A, HLA-B, and HLA-C, by knocking out a gene encoding said molecule. In some embodiments, the gene encoding an HLA-A protein is knocked out to reduce or eliminate expression of said HLA-A protein. In some embodiments, the gene encoding an HLA-B protein is knocked out to reduce or eliminate expression of said HLA-B protein. In some embodiments, the gene encoding an HLA-C protein is knocked out to reduce or eliminate expression of said HLA-C protein.

In some embodiments, the engineered islets comprise a modification (e.g., genetic modification) targeting the B2M gene. In some embodiments, the modification (e.g., genetic modification) targeting the B2M gene is by using a targeted nuclease system that comprises a Cas protein or a polynucleotide encoding a Cas protein, and at least one guide ribonucleic acid sequence for specifically targeting the B2M gene. In some embodiments, the at least one guide ribonucleic acid sequence (e.g. gRNA targeting sequence) for specifically targeting the B2M gene is selected from the group consisting of SEQ ID NOS: 81240-85644 of Appendix 2 or Table 15 of WO2016/183041, the disclosure is incorporated by reference in its entirety. In some embodiments, the gRNA targeting sequence for specifically targeting the B2M gene is CGUGAGUAAACCUGAAUCUU (SEQ ID NO: 29).

In some embodiments, an exogenous nucleic acid or transgene encoding a polypeptide as disclosed herein (e.g., a chimeric antigen receptor, CD47, or another tolerogenic factor disclosed herein) is inserted at the B2M gene. Exemplary transgenes for targeted insertion at the B2M locus include any as described herein.

Assays to test whether the B2M gene has been inactivated are known and described herein. In one embodiment, the resulting genetic modification of the B2M gene is assessed by PCR. In some embodiments, the reduction of one or more MHC class I, such as HLA-I, expression can be assays by flow cytometry, such as by FACS analysis. In another embodiment, B2M protein expression is detected using a Western blot of cells lysates probed with antibodies to the B2M protein. In another embodiment, reverse transcriptase polymerase chain reactions (RT-PCR) are used to confirm the presence of the inactivating modification, such as genetic modification. In some embodiments, the reduction in one or more MHC class I molecules expression is assessed using an immunoaffinity technique, such as immunohistochemistry or immunocytochemistry.

In some embodiments, the reduction of one or more MHC class I molecules expression or function (HLA I when the cells are derived from human cells) in the engineered islets can be measured using techniques known in the art; for example, FACS techniques using labeled antibodies that bind the HLA complex; for example, using commercially available HLA-A, B, C antibodies that bind to the alpha chain of the human major histocompatibility HLA Class I antigens. In addition, the cells can be tested to confirm that the HLA I complex is not expressed on the cell surface. This may be assayed by FACS analysis using antibodies to one or more HLA cell surface components as discussed above. In addition to the reduction of HLA I (or MHC class I), the engineered islets provided herein have a reduced susceptibility to macrophage phagocytosis and NK cell killing. Methods to assay for hypoimmunogenic phenotypes of the engineered islets are described further below.

In some embodiments, the modification (e.g., genetic modification) that reduces B2M expression reduces B2M mRNA expression. In some embodiments, the reduced mRNA expression of B2M is relative to an unmodified or wild-type cell of the same cell type that does not comprise the modification. In some embodiments, the mRNA expression of B2M is reduced by more than about 5%, such as reduced by more than about any of 10%, 20%, 30%, 40%, 50%, 60%, 70%, 80%, 90%, or more. In some embodiments, the mRNA expression of B2M is reduced by up to about 100%, such as reduced by up to about any of 90%, 80%, 70%, 60%, 50%, 40%, 30%, 20%, 10%, 5%, or less. In some embodiments, the mRNA expression of B2M is reduced by any of about 5%, 10%, 20%, 30%, 40%, 50%, 60%, 70%, 80%, 90%, or 100%. In some embodiments, the mRNA expression of B2M is eliminated (e.g., 0% expression of B2M mRNA). In some embodiments, the modification that reduces B2M mRNA expression eliminates B2M gene activity.

In some embodiments, the modification (e.g., genetic modification) that reduces B2M expression reduces B2M protein expression. In some embodiments, the reduced protein expression of B2M is relative to an unmodified or wild-type cell of the same cell type that does not comprise the modification. In some embodiments, the protein expression of B2M is reduced by more than about 5%, such as reduced by more than about any of 10%, 20%, 30%, 40%, 50%, 60%, 70%, 80%, 90%, or more. In some embodiments, the protein expression of B2M is reduced by up to about 100%, such as reduced by up to about any of 90%, 80%, 70%, 60%, 50%, 40%, 30%, 20%, 10%, 5%, or less. In some embodiments, the protein expression of B2M is reduced by any of about 5%, 10%, 20%, 30%, 40%, 50%, 60%, 70%, 80%, 90%, or 100%. In some embodiments, the protein expression of B2M is eliminated (e.g., 0% expression of B2M protein). In some embodiments, the modification that reduces B2M protein expression eliminates B2M gene activity.

In some embodiments, the modification (e.g., genetic modification) that reduces B2M expression comprises inactivation or disruption of the B2M gene. In some embodiments, the modification that reduces B2M expression comprises inactivation or disruption of one allele of the B2M gene. In some embodiments, the modification that reduces B2M expression comprises inactivation or disruption comprises inactivation or disruption of both alleles of the B2M gene.

In some embodiments, the modification (e.g., genetic modification) comprises inactivation or disruption of one or more B2M coding sequences in the cell. In some embodiments, the modification comprises inactivation or disruption of all B2M coding sequences in the cell. In some embodiments, the modification comprises inactivation or disruption comprises an indel in the B2M gene. In some embodiments, the modification is a frameshift mutation of genomic DNA of the B2M gene. In some embodiments, the modification is a deletion of genomic DNA of the B2M gene. In some embodiments, the modification is a deletion of a contiguous stretch of genomic DNA of the B2M gene. In some embodiments, the B2M gene is knocked out.

a. MHC Class II Molecules

In certain aspects, the modification, such as genetic modification, reduces or eliminates, such as knocks out, the expression of one or more MHC class II molecules genes by targeting Class II molecules transactivator (CIITA) expression. In some embodiments, the genetic modification occurs using a CRISPR/Cas system. CIITA is a member of the LR or nucleotide binding domain (NBD) leucine-rich repeat (LRR) family of proteins and regulates the transcription of one or more MHC class II molecules by associating with the MHC enhanceosome. By reducing or eliminating, such as knocking out, expression of CIITA, expression of one or more MHC class II molecules is reduced thereby also reducing surface expression. In some cases, such cells exhibit immune tolerance when engrafted into a recipient subject. In some embodiments, the cell is considered hypoimmunogenic, e.g., in a recipient subject or patient upon administration.

In some embodiments, the target polynucleotide sequence is a variant of CIITA. In some embodiments, the target polynucleotide sequence is a homolog of CIITA. In some embodiments, the target polynucleotide sequence is an ortholog of CIITA.

In some embodiments, decreased or eliminated expression of one or more MHC class II molecules is a modification that reduces expression of one or more of the following MHC class II molecules—HLA-DP, HLA-DM, HLA-DOA, HLA-DOB, HLA-DQ, and HLA-DR. In some embodiments, reduced or eliminated expression of CIITA reduces or eliminates expression of one or more of the following MHC class II molecules—HLA-DP, HLA-DM, HLA-DOA, HLA-DOB, HLA-DQ, and HLA-DR. In some embodiments, decreased or eliminated expression of CIITA reduces or eliminates expression of an HLA-DP protein. In some embodiments, decreased or eliminated expression of CIITA reduces or eliminates expression of an HLA-DM protein. In some embodiments, decreased or eliminated expression of CIITA reduces or eliminates expression of an HLA-DOA protein. In some embodiments, decreased or eliminated expression of CIITA reduces or eliminates expression of an HLA-DOB protein. In some embodiments, decreased or eliminated expression of CIITA reduces or eliminates expression of an HLA-DQ protein. In some embodiments, decreased or eliminated expression of CIITA reduces or eliminates expression of an HLA-DR protein. In some embodiments, decreased or eliminated expression of CIITA reduces or eliminates expression of one or more of the following MHC class II molecules—HLA-DP, HLA-DM, HLA-DOA, HLA-DOB, HLA-DQ, and HLA-DR, by knocking out a gene encoding said molecule. In some embodiments, the gene encoding an HLA-DP protein is knocked out to reduce or eliminate expression of said HLA-DP protein. In some embodiments, the gene encoding an HLA-DM protein is knocked out to reduce or eliminate expression of said HLA-DM protein. In some embodiments, the gene encoding an HLA-DOA protein is knocked out to reduce or eliminate expression of said HLA-DOA protein. In some embodiments, the gene encoding an HLA-DOB protein is knocked out to reduce or eliminate expression of said HLA-DOB protein. In some embodiments, the gene encoding an HLA-DQ protein is knocked out to reduce or eliminate expression of said HLA-DQ protein. In some embodiments, the gene encoding an HLA-DR protein is knocked out to reduce or eliminate expression of said HLA-DR protein.

In some embodiments, the engineered islets comprise a modification (e.g., genetic modification) targeting the CIITA gene. In some embodiments, the modification targeting the CIITA gene is by a targeted nuclease system that comprises a Cas protein or a polynucleotide encoding a Cas protein, and at least one guide ribonucleic acid sequence for specifically targeting the CIITA gene. In some embodiments, the at least one guide ribonucleic acid sequence (e.g. gRNA targeting sequence) for specifically targeting the CIITA gene is selected from the group consisting of SEQ ID NOS: 5184-36352 of Appendix 1 or Table 12 of WO2016183041, the disclosure is incorporated by reference in its entirety. In some embodiments, the gRNA targeting sequence for specifically targeting the CIITA gene is GAUAUUGGCAUAAGCCUCCC (SEQ ID NO: 30).

In some embodiments, an exogenous nucleic acid or transgene encoding a polypeptide as disclosed herein (e.g., a chimeric antigen receptor, CD47, or another tolerogenic factor disclosed herein) is inserted at the CIITA gene. Exemplary transgenes for targeted insertion at the B2M locus include any as described in herein.

Assays to test whether the CIITA gene has been inactivated are known and described herein. In one embodiment, the resulting genetic modification of the CIITA gene is assessed by PCR. In some embodiments, the reduction of one or more MHC class II molecules, such as HLA-II, expression can be assays by flow cytometry, such as by FACS analysis. In another embodiment, CIITA protein expression is detected using a Western blot of cells lysates probed with antibodies to the CIITA protein. In another embodiment, reverse transcriptase polymerase chain reactions (RT-PCR) are used to confirm the presence of the inactivating modification, such as genetic modification. In some embodiments, the reduction in one or more MHC class II molecules expression is assessed using an immunoaffinity technique, such as immunohistochemistry or immunocytochemistry.

In some embodiments, the reduction of the one or more MHC class II molecules expression or function (HLA II when the cells are derived from human cells) in the engineered islets can be measured using techniques known in the art, such as Western blotting using antibodies to the protein, FACS techniques, RT-PCR techniques, etc. In some embodiments, the engineered islets can be tested to confirm that the HLA II complex is not expressed on the cell surface. Methods to assess surface expression include methods known in the art (See FIG. 21 of WO2018132783, for example) and generally is done using either Western Blots or FACS analysis based on commercial antibodies that bind to human HLA Class II HLA-DR, DP and most DQ antigens. In addition to the reduction of one or more HLA class II molecules (or one or more MHC class II molecules), the engineered islets provided herein have a reduced susceptibility to macrophage phagocytosis and NK cell killing. Methods to assay for hypoimmunogenic phenotypes of the engineered islets are described further below.

In some embodiments, the modification (e.g., genetic modification) that reduces CIITA expression reduces CIITA mRNA expression. In some embodiments, the reduced mRNA expression of CIITA is relative to an unmodified or wild-type cell of the same cell type that does not comprise the modification. In some embodiments, the mRNA expression of CIITA is reduced by more than about 5%, such as reduced by more than about any of 10%, 20%, 30%, 40%, 50%, 60%, 70%, 80%, 90%, or more. In some embodiments, the mRNA expression of CIITA is reduced by up to about 100%, such as reduced by up to about any of 90%, 80%, 70%, 60%, 50%, 40%, 30%, 20%, 10%, 5%, or less. In some embodiments, the mRNA expression of CIITA is reduced by any of about 5%, 10%, 20%, 30%, 40%, 50%, 60%, 70%, 80%, 90%, or 100%. In some embodiments, the mRNA expression of CIITA is eliminated (e.g., 0% expression of CIITA mRNA). In some embodiments, the modification that reduces CIITA mRNA expression eliminates CIITA gene activity.

In some embodiments, the modification (e.g., genetic modification) that reduces CIITA expression reduces CIITA protein expression. In some embodiments, the reduced protein expression of CIITA is relative to an unmodified or wild-type cell of the same cell type that does not comprise the modification. In some embodiments, the protein expression of CIITA is reduced by more than about 5%, such as reduced by more than about any of 10%, 20%, 30%, 40%, 50%, 60%, 70%, 80%, 90%, or more. In some embodiments, the protein expression of CIITA is reduced by up to about 100%, such as reduced by up to about any of 90%, 80%, 70%, 60%, 50%, 40%, 30%, 20%, 10%, 5%, or less. In some embodiments, the protein expression of CIITA is reduced by any of about 5%, 10%, 20%, 30%, 40%, 50%, 60%, 70%, 80%, 90%, or 100%. In some embodiments, the protein expression of CIITA is eliminated (e.g., 0% expression of CIITA protein). In some embodiments, the modification that reduces CIITA protein expression eliminates CIITA gene activity.

In some embodiments, the modification (e.g., genetic modification) that reduces CIITA expression comprises inactivation or disruption of the CIITA gene. In some embodiments, the modification that reduces CIITA expression comprises inactivation or disruption of one allele of the CIITA gene. In some embodiments, the modification that reduces CIITA expression comprises inactivation or disruption comprises inactivation or disruption of both alleles of the CIITA gene.

In some embodiments, the modification F (e.g., genetic modification) comprises inactivation or disruption of one or more CIITA coding sequences in the cell. In some embodiments, the modification comprises inactivation or disruption of all CIITA coding sequences in the cell. In some embodiments, the modification comprises inactivation or disruption comprises an indel in the CIITA gene. In some embodiments, the modification is a frameshift mutation of genomic DNA of the CIITA gene. In some embodiments, the modification is a deletion of genomic DNA of the CIITA gene. In some embodiments, the modification is a deletion of a contiguous stretch of genomic DNA of the CIITA gene. In some embodiments, the CIITA gene is knocked out.

### B. Overexpression of Polynucleotides

In some embodiments, the engineered islets is genetically modified or engineered, such as by introduction of one or more modifications into a beta cell to overexpress a desired polynucleotide in the cell. In some embodiments, the islet cells to be modified or engineered is an unmodified cell or non-engineered cell (e.g. unengineered islets or non-engineered islet cells, e.g., control or wild-type cell) that has not previously been introduced with the one or more modifications. In some embodiments, the engineered islet cells are genetically modified to include one or more exogenous polynucleotides encoding an exogenous protein (also interchangeably used with the term “transgene”). As described, in some embodiments, the engineered islet cells are modified to increase expression of certain genes that are tolerogenic (e.g., immune) factors that affect immune recognition and tolerance in a recipient. The one or more polynucleotides, e.g. exogenous polynucleotides, may be expressed (e.g. overexpressed) in the engineered islets together with one or more genetic modifications to reduce expression of a target polynucleotide described herein, such as an one or more MHC class I molecules and/or one or more MHC class II molecules. In some embodiments, the provided engineered islets do not trigger or activate an immune response upon administration to a recipient subject.

In some embodiments, the engineered islets includes 1, 2, 3, 4, 5, 6, 7, 8, 9, 10 or more different overexpressed polynucleotides. In some embodiments, the engineered islets includes 1, 2, 3, 4, 5, 6, 7, 8, 9, 10 or more different overexpressed polynucleotides. In some embodiments, the overexpressed polynucleotide is an exogenous polynucleotide. In some embodiments, the engineered islets includes 1, 2, 3, 4, 5, 6, 7, 8, 9, 10 or more different exogenous polynucleotides. In some embodiments, the engineered islets includes 1, 2, 3, 4, 5, 6, 7, 8, 9, 10 or more different exogenous polynucleotides. In some embodiments, the overexpressed polynucleotide is an exogenous polynucleotide that is expressed episomally in the beta cell. In some embodiments, the overexpressed polynucleotide is an exogenous polynucleotide that is inserted or integrated into one or more genomic loci of the engineered islets.

In some embodiments, expression of a polynucleotide is increased, i.e. the polynucleotide is overexpressed, using a fusion protein containing a DNA-targeting domain and a transcriptional activator. Targeted methods of increasing expression using transactivator domains are known to a skilled artisan.

In some embodiments, the modified beta contains one or more exogenous polynucleotides in which the one or more exogenous polynucleotides are inserted or integrated into a genomic locus of the cell by non-targeted insertion methods, such as by transduction with a lentiviral vector. In some embodiments, the lentiviral vector comprises a piggyBac transposon. During transposition, the piggyback transposon recognizes transposon-specific inverted terminal repeats (ITRs) in a lentiviral vector, to allow for the efficient movement and integration of the vector contents into TTAA chromosomal sites. In some embodiments, the one or more exogenous polynucleotides are inserted or integrated into the genome of the cell, such as beta cell, by targeted insertion methods, such as by using homology directed repair (HDR). Any suitable method can be used to insert the exogenous polynucleotide into the genomic locus of the modified cell, such as beta cell, by HDR including the gene editing methods described herein (e.g., a CRISPR/Cas system). In some embodiments, the one or more exogenous polynucleotides are inserted into one or more genomic locus, such as any genomic locus described herein (e.g. Table 4). In some embodiments, the exogenous polynucleotides are inserted into the same genomic loci. In some embodiments, the exogenous polynucleotides are inserted into different genomic loci. In some embodiments, the two or more of the exogenous polynucleotides are inserted into the same genomic loci, such as any genomic locus described herein (e.g. Table 4). In some embodiments, two or more exogenous polynucleotides are inserted into a different genomic loci, such as two or more genomic loci as described herein (e.g., Table 4).

Exemplary polynucleotides or overexpression, and methods for overexpressing the same, are described in the following subsections.

### 1. Tolerogenic Factor

In some embodiments, expression of a tolerogenic factor is overexpressed or increased in the cell, e.g. engineered islets. In some embodiments, the engineered islets includes increased expression, i.e. overexpression, of at least one tolerogenic factor. In some embodiments, the tolerogenic factor is any factor that promotes or contributes to promoting or inducing tolerance to the engineered islets by the immune system (e.g. innate or adaptive immune system).

In some embodiments, the one or more tolerogenic factors is selected from the group consisting of CD47, A20/TNFAIP3, Cl-Inhibitor, CCL21, CCL22, CD16, CD16 Fc receptor, CD24, CD27, CD35, CD39, CD46, CD52, CD55, CD59, CD200, CR1, CTLA4-Ig, DUX4, FasL, H2-M3, HLA-C, HLA-E, HLA-E heavy chain, HLA-G, PD-L1, IDO1, IL-10, IL15-RF, IL-35, MANF, Mfge8, PD-1, PD-L1, or Serpinb9. In some embodiments, the tolerogenic factor is DUX4, B2M-HLA-E, CD35, CD52, CD16, CD52, CD47, CD46, CD55, CD59, CD27, CD200, HLA-C, HLA-E, HLA-E heavy chain, HLA-G, PD-L1, IDO1, CTLA4-Ig, Cl-Inhibitor, IL-10, IL-35, FASL, CCL21, MFGE8, SERPINB9, CD35, IL-39, CD16 Fc Receptor, IL15-RF, and H2-M3. In some embodiments, the tolerogenic factor is CD47, PD-L1, HLA-E or HLA-G, CCL21, FasL, Serpinb9, CD200 or Mfge8, or any combination thereof. In some embodiments, the cell, such as a beta cell, includes at least one exogenous polynucleotide that includes a polynucleotide that encodes for a tolerogenic factor. For instance, in some embodiments, at least one of the exogenous polynucleotides is a polynucleotide that encodes CD47. Provided herein are cells that do not trigger or activate an immune response upon administration to a recipient subject. As described above, in some embodiments, the cells, such as beta cells, are modified to increase expression of genes and tolerogenic (e.g., immune) factors that affect immune recognition and tolerance in a recipient.

In some embodiments, the expression (e.g., surface expression) of a tolerogenic factor is increased by about 10% or higher compared to a cell of the same cell type that does not comprise the modification, such as increased by any of about 15%, 20%, 25%, 30%, 35%, 40%, 45%, 50%, 55%, 60%, 65%, 70%, 75%, 80%, 85%, 90%, 95%, 99%, or higher, compared to a cell of the same cell type that does not comprise the modification. In some embodiments, the expression of a tolerogenic factor is increased by about 99% or lower compared to a cell of the same cell type that does not comprise the modification, such as increased by any of about 95%, 90%, 85%, 80%, 75%, 70%, 65%, 60%, 55%, 50%, 45%, 40%, 35%, 30%, 25%, 20%, 15%, 10%, or lower, compared to a cell of the same cell type that does not comprise the modification. In some embodiments, the expression of a tolerogenic factor is increased by between about 10% and about 100% compared to a cell of the same cell type that does not comprise the modification, such as between any of about 10% and about 40%, about 20% and about 60%, about 50% and about 80%, and about 70% and about 100%, compared to a cell of the same cell type that does not comprise the modification.

In some embodiments, the expression (e.g., surface expression) of a tolerogenic factor is increased by about 2-fold or higher compared to a cell of the same cell type that does not comprise the modification, such as any of about 4-fold or higher, 6-fold or higher, 8-fold or higher, 10-fold or higher, 15-fold or higher, 20-fold or higher, 30-fold or higher, 40-fold or higher, 50-fold or higher, 60-fold or higher, 70-fold or higher, 80-fold or higher, 90-fold or higher, 100-fold or higher, 150-fold or higher, and 200-fold or higher compared to a cell of the same cell type that does not comprise the modification. In some embodiments, the expression of a tolerogenic factor is increased by about 200-fold or lower compared to a cell of the same cell type that does not comprise the modification, such as any of about 150-fold or lower, 100-fold or lower, 90-fold or lower, 80-fold or lower, 70-fold or lower, 60-fold or lower, 50-fold or lower, 40-fold or lower, 30-fold or lower, 15-fold or lower, 10-fold or lower, 8-fold or lower, 6-fold or lower, 4-fold or lower, and 2-fold or lower compared to a cell of the same cell type that does not comprise the modification. In some embodiments, the expression of a tolerogenic factor is increased by between about 2-fold and about 200-fold compared to a cell of the same cell type that does not comprise the modification, such as between any of about 2-fold and about 20-fold, about 10-fold and about 50-fold, about 30-fold and about 70-fold, about 50-fold and about 100-fold, about 80-fold and about 150-fold, and about 120-fold and about 200-fold, compared to a cell of the same cell type that does not comprise the modification.

In some embodiments, the present disclosure provides a cell, such as a beta cell, or population thereof that has been modified to express the tolerogenic factor (e.g., immunomodulatory polypeptide), such as CD47. In some embodiments, the present disclosure provides a method for altering a cell genome to express the tolerogenic factor (e.g. immunomodulatory polypeptide), such as CD47. In some embodiments, the engineered islets expresses an exogenous tolerogenic factor (e.g. immunomodulatory polypeptide), such as an exogenous CD47. In some instances, overexpression or increasing expression of the exogenous polynucleotide is achieved by introducing into the beta cell (e.g. transducing the cell) with an expression vector comprising a nucleotide sequence encoding a human CD47 polypeptide. In some embodiments, the expression vector may be a viral vector, such as a lentiviral vector) or may be a non-viral vector. In some embodiments, the cell, such as a beta cell, is modified to contain one or more exogenous polynucleotides in which at least one of the exogenous polynucleotides includes a polynucleotide that encodes for a tolerogenic factor. In some embodiments, the DUX4, B2M-HLA-E, CD35, CD52, CD16, CD52, CD47, CD46, CD55, CD59, CD27, CD200, HLA-C, HLA-E, HLA-E heavy chain, HLA-G, PD-L1, IDO1, CTLA4-Ig, Cl-Inhibitor, IL-10, IL-35, FASL, CCL21, MFGE8, SERPINB9, CD35, IL-39, CD16 Fc Receptor, IL15-RF, and H2-M3 (including any combination thereof). In some embodiments, the tolerogenic factor is one or more of CD47, PD-L1, HLA-E or HLA-G, CCL21, FasL, Serpinb9, CD200, and Mfge8 (including any combination thereof). For instance, in some embodiments, at least one of the exogenous polynucleotides is a polynucleotide that encodes CD47.

In some embodiments, the tolerogenic factor is CD47. In some embodiments, the engineered islets contains an exogenous polynucleotide that encodes CD47, such as human CD47. In some embodiments, CD47 is overexpressed in the cell. In some embodiments, the expression of CD47 is overexpressed or increased in the engineered islets compared to a similar cell of the same cell type that has not been modified with the modification, such as a reference or unmodified cell, e.g. a beta cell not modified with an exogenous polynucleotide encoding CD47. CD47 is a leukocyte surface antigen and has a role in cell adhesion and modulation of integrins. It is normally expressed on the surface of a cell and signals to circulating macrophages not to eat the cell. Useful genomic, polynucleotide and polypeptide information about human CD47 are provided in, for example, the NP_001768.1, NP_942088.1, NM_001777.3 and NM_198793.2.

In some embodiments, the expression (e.g., surface expression) of CD47 is increased by about 10% or higher compared to a cell of the same cell type that does not comprise the modification, such as increased by any of about 15%, 20%, 25%, 30%, 35%, 40%, 45%, 50%, 55%, 60%, 65%, 70%, 75%, 80%, 85%, 90%, 95%, 99%, or higher, compared to a cell of the same cell type that does not comprise the modification. In some embodiments, the expression of CD47 is increased by about 99% or lower compared to a cell of the same cell type that does not comprise the modification, such as increased by any of about 95%, 90%, 85%, 80%, 75%, 70%, 65%, 60%, 55%, 50%, 45%, 40%, 35%, 30%, 25%, 20%, 15%, 10%, or lower, compared to a cell of the same cell type that does not comprise the modification. In some embodiments, the expression of CD47 is increased by between about 10% and about 100% compared to a cell of the same cell type that does not comprise the modification, such as between any of about 10% and about 40%, about 20% and about 60%, about 50% and about 80%, and about 70% and about 100%, compared to a cell of the same cell type that does not comprise the modification.

In some embodiments, the expression (e.g., surface expression) of CD47 is increased in the engineered islets by about 2-fold or higher compared to a cell of the same cell type that does not comprise the modification, such as any of about 4-fold or higher, 6-fold or higher, 8-fold or higher, 10-fold or higher, 15-fold or higher, 20-fold or higher, 30-fold or higher, 40-fold or higher, 50-fold or higher, 60-fold or higher, 70-fold or higher, 80-fold or higher, 90-fold or higher, 100-fold or higher, 150-fold or higher, and 200-fold or higher compared to a cell of the same cell type that does not comprise the modification. In some embodiments, the expression of CD47 is increased by about 200-fold or lower compared to a cell of the same cell type that does not comprise the modification, such as any of about 150-fold or lower, 100-fold or lower, 90-fold or lower, 80-fold or lower, 70-fold or lower, 60-fold or lower, 50-fold or lower, 40-fold or lower, 30-fold or lower, 15-fold or lower, 10-fold or lower, 8-fold or lower, 6-fold or lower, 4-fold or lower, and 2-fold or lower compared to a cell of the same cell type that does not comprise the modification. In some embodiments, the expression of CD47 is increased by between about 2-fold and about 200-fold compared to a cell of the same cell type that does not comprise the modification, such as between any of about 2-fold and about 20-fold, about 10-fold and about 50-fold, about 30-fold and about 70-fold, about 50-fold and about 100-fold, about 80-fold and about 150-fold, and about 120-fold and about 200-fold, compared to a cell of the same cell type that does not comprise the modification.

In some embodiments, the engineered islets comprise a nucleotide sequence encoding a CD47 polypeptide has at least 95% sequence identity (e.g., 95%, 96%, 97%, 98%, 99%, or more) to an amino acid sequence as set forth in NCBI Ref Sequence Nos. NP_001768.1 and NP_942088.1. In some embodiments, the engineered islets comprise a nucleotide sequence encoding a CD47 polypeptide having an amino acid sequence as set forth in NCBI Ref Sequence Nos. NP_001768.1 and NP_942088.1. In some embodiments, the engineered islets comprise a nucleotide sequence for CD47 having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref Nos. NM_001777.3 and NM_198793.2. In some embodiments, the engineered islets comprise a nucleotide sequence for CD47 as set forth in NCBI Ref. Sequence Nos. NM_001777.3 and NM_198793.2.

In some embodiments, the engineered islets comprise a CD47 polypeptide having at least 95% sequence identity (e.g., 95%, 96%, 97%, 98%, 99%, or more) to an amino acid sequence as set forth in NCBI Ref Sequence Nos. NP_001768.1 and NP_942088.1. In some embodiments, the engineered islets comprise a CD47 polypeptide having an amino acid sequence as set forth in NCBI Ref Sequence Nos. NP_001768.1 and NP_942088.1.

In some embodiments, the engineered islets comprise a CD47 polypeptide having at least 95% sequence identity (e.g., 95%, 96%, 97%, 98%, 99%, or more) to an amino acid sequence as set forth in SEQ ID NO: 1. In some embodiments, the engineered islets comprise a CD47 polypeptide having the amino acid sequence as set forth in SEQ ID NO: 1. In some embodiments, the engineered islets comprise a CD47 polypeptide having at least 95% sequence identity (e.g., 95%, 96%, 97%, 98%, 99%, or more) to an amino acid sequence as set forth in SEQ ID NO: 2. In some embodiments, the engineered islets comprise a CD47 polypeptide having the amino acid sequence as set forth in SEQ ID NO: 2.

In certain embodiments, the polynucleotide encoding CD47 is operably linked to a promoter.

In some embodiments, an exogenous polynucleotide encoding CD47 is integrated into the genome of the engineered islets by targeted or non-targeted methods of insertion, such as described further below. In some embodiments, targeted insertion is by homology-dependent insertion into a target loci, such as by insertion into any one of the genomic (gene) loci. In some embodiments, each of the one or more genomic loci are selected from the group consisting of a MICA gene locus, a MICB gene locus, a B2M gene locus, a CIITA gene locus, a CD142 gene locus, a CCR5 gene locus, CXCR4 gene locus, PPP1R12C (also known as AAVS1) gene locus, albumin gene locus, SHS231 locus, CLYBL gene locus, ROSA26 gene locus, LRP1 gene locus, HMGB1 gene locus, ABO gene locus, RHD gene locus, FUT1 gene locus, and KDM5D gene locus. In some embodiments, each of the one or more genomic loci are selected from the group consisting of a B2M locus, a TAP1 locus, a CIITA locus, a MIC-A locus, a MIC-B locus, and a safe harbor locus. In some embodiments, the safe harbor locus is selected from the group consisting of an AAVS1, ABO, CCR5, CLYBL, CXCR4, F3, FUT1, HMGB1, KDM5D, LRP1, MICA, MICB, RHD, ROSA26, and SHS231 locus.

In some embodiments, targeted insertion is by homology-dependent insertion into a target loci, such as by insertion into any one of the gene loci described herein, e.g. a B2M gene, a CIITA gene. In some embodiments, targeted insertion is by homology-independent insertion, such as by insertion into a safe harbor locus. In some cases, the polynucleotide encoding CD47 is inserted into a safe harbor locus, such as but not limited to, a gene locus selected from AAVS1, CCR5, CLYBL, ROSA26, and SHS231. In particular embodiments, the polynucleotide encoding CD47 is inserted into the CCR5 gene locus, the PPP1R12C (also known as AAVS1) gene locus or the CLYBL gene locus. In some embodiments, the polynucleotide encoding CD47 is inserted into any one of the gene loci described herein. In some cases, the polynucleotide encoding CD47 is inserted into a safe harbor locus.

In particular embodiments, the polynucleotide encoding CD47 is inserted into the CCR5 gene locus, the PPP1R12C (also known as AAVS1) gene locus or the CLYBL gene locus. In some embodiments, the polynucleotide encoding CD47 is inserted into a B2M gene locus or a CIITA gene locus. In some embodiments, a suitable gene editing system (e.g., CRISPR/Cas system or any of the gene editing systems described herein) is used to facilitate the insertion of a polynucleotide encoding CD47, into a genomic locus of the cell.

In some embodiments, CD47 protein expression is detected using a Western blot of cell lysates probed with antibodies against the CD47 protein. In another embodiment, reverse transcriptase polymerase chain reactions (RT-PCR) are used to confirm the presence of the exogenous CD47 mRNA.

In some embodiments, the engineered islets contains an exogenous polynucleotide that encodes CD200, such as human CD200. In some embodiments, CD200 is overexpressed in the cell. In some embodiments, the expression of CD200 is increased in the engineered islets compared to a similar reference or unmodified cell (including with any other modifications, such as genetic modifications) except that the reference or unmodified cell does not include the exogenous polynucleotide encoding CD200. Useful genomic, polynucleotide and polypeptide information about human CD200 are provided in, for example, the GeneCard Identifier GC03P 112332, HGNC No. 7203, NCBI Gene ID 4345, Uniprot No. P41217, and NCBI RefSeq Nos. NP_001004196.2, NM 001004196.3, NP_001305757.1, NM 001318828.1, NP_005935.4, NM 005944.6, XP_005247539.1, and XM_005247482.2. In certain embodiments, the polynucleotide encoding CD200 is operably linked to a promoter.

In some embodiments, the polynucleotide encoding CD200 is inserted into any one of the gene loci described herein. In some cases, the polynucleotide encoding CD200 is inserted into a safe harbor locus, such as but not limited to, a gene locus selected from AAVS1, CCR5, CLYBL, ROSA26, and SHS231. In particular embodiments, the polynucleotide encoding CD200 is inserted into the CCR5 gene locus, the PPP1R12C (also known as AAVS1) gene locus or the CLYBL gene locus. In some embodiments, the polynucleotide encoding CD200 is inserted into a B2M gene locus or a CIITA gene locus. In some embodiments, a suitable gene editing system (e.g., CRISPR/Cas system or any of the gene editing systems described herein) is used to facilitate the insertion of a polynucleotide encoding CD200, into a genomic locus of the cell.

In some embodiments, CD200 protein expression is detected using a Western blot of cell lysates probed with antibodies against the CD200 protein. In another embodiment, reverse transcriptase polymerase chain reactions (RT-PCR) are used to confirm the presence of the exogenous CD200 mRNA.

In some embodiments, the engineered islets contains an exogenous polynucleotide that encodes HLA-E, such as human HLA-E. In some embodiments, HLA-E is overexpressed in the cell. In some embodiments, the expression of HLA-E is increased in the engineered islets compared to a similar reference or unmodified cell (including with any other modifications) except that the reference or unmodified cell does not include the exogenous polynucleotide encoding HLA-E. Useful genomic, polynucleotide and polypeptide information about human HLA-E are provided in, for example, the GeneCard Identifier GC06P047281, HGNC No. 4962, NCBI Gene ID 3133, Uniprot No. P13747, and NCBI RefSeq Nos. NP_005507.3 and NM_005516.5. In certain embodiments, the polynucleotide encoding HLA-E is operably linked to a promoter.

In some embodiments, the polynucleotide encoding HLA-E is inserted into any one of the gene loci described herein. In some cases, the polynucleotide encoding HLA-E is inserted into a safe harbor locus, such as but not limited to, a gene locus selected from AAVS1, CCR5, CLYBL, ROSA26, SHS231. In particular embodiments, the polynucleotide encoding HLA-E is inserted into the CCR5 gene locus, the PPP1R12C (also known as AAVS1) gene locus or the CLYBL gene locus. In some embodiments, the polynucleotide encoding HLA-E is inserted into a B2M gene locus, a CIITA gene locus. In some embodiments, a suitable gene editing system (e.g., CRISPR/Cas system or any of the gene editing systems described herein) is used to facilitate the insertion of a polynucleotide encoding HLA-E, into a genomic locus of the cell.

In some embodiments, HLA-E protein expression is detected using a Western blot of cell lysates probed with antibodies against the HLA-E protein. In another embodiment, reverse transcriptase polymerase chain reactions (RT-PCR) are used to confirm the presence of the exogenous HLA-E mRNA.

In some embodiments, the engineered islets contains an exogenous polynucleotide that encodes HLA-G, such as human HLA-G. In some embodiments, HLA-G is overexpressed in the cell. In some embodiments, the expression of HLA-G is increased in the engineered islets compared to a similar reference or unmodified cell (including with any other modifications, such as genetic modifications) except that the reference or unmodified cell does not include the exogenous polynucleotide encoding HLA-G. Useful genomic, polynucleotide and polypeptide information about human HLA-G are provided in, for example, the GeneCard Identifier GC06P047256, HGNC No. 4964, NCBI Gene ID 3135, Uniprot No. P17693, and NCBI RefSeq Nos. NP_002118.1 and NM_002127.5. In certain embodiments, the polynucleotide encoding HLA-G is operably linked to a promoter.

In some embodiments, the polynucleotide encoding HLA-G is inserted into any one of the gene loci described herein. In some cases, the polynucleotide encoding HLA-G is inserted into a safe harbor locus, such as but not limited to, a gene locus selected from AAVS1, CCR5, CLYBL, ROSA26, and SHS231. In particular embodiments, the polynucleotide encoding HLA-G is inserted into the CCR5 gene locus, the PPP1R12C (also known as AAVS1) gene locus or the CLYBL gene locus. In some embodiments, the polynucleotide encoding HLA-G is inserted into a B2M gene locus or; a CIITA gene locus. In some embodiments, a suitable gene editing system (e.g., CRISPR/Cas system or any of the gene editing systems described herein) is used to facilitate the insertion of a polynucleotide encoding HLA-G, into a genomic locus of the cell.

In some embodiments, HLA-G protein expression is detected using a Western blot of cell lysates probed with antibodies against the HLA-G protein. In another embodiment, reverse transcriptase polymerase chain reactions (RT-PCR) are used to confirm the presence of the exogenous HLA-G mRNA.

In some embodiments, the engineered islets contains an exogenous polynucleotide that encodes PD-L1, such as human PD-L1. In some embodiments, PD-L1 is overexpressed in the cell. In some embodiments, the expression of PD-L1 is increased in engineered islets compared to a similar reference or unmodified cell (including with any other modifications) except that the reference or unmodified cell does not include the exogenous polynucleotide encoding PD-L1. Useful genomic, polynucleotide and polypeptide information about human PD-L1 or CD274 are provided in, for example, the GeneCard Identifier GC09P005450, HGNC No. 17635, NCBI Gene ID 29126, Uniprot No. Q9NZQ7, and NCBI RefSeq Nos. NP_001254635.1, NM_001267706.1, NP_054862.1, and NM_014143.3. In certain embodiments, the polynucleotide encoding PD-L1 is operably linked to a promoter.

In some embodiments, the polynucleotide encoding PD-L1 is inserted into any one of the gene loci described herein. In some cases, the polynucleotide encoding PD-L1 is inserted into a safe harbor locus, such as but not limited to, a gene locus selected from AAVS1, CCR5, CLYBL, ROSA26, and SHS231. In particular embodiments, the polynucleotide encoding PD-L1 is inserted into the CCR5 gene locus, the PPPIR12C (also known as AAVS1) gene locus or the CLYBL gene locus. In some embodiments, the polynucleotide encoding PD-L1 is inserted into a B2M gene locus, a CIITA gene locus. In some embodiments, a suitable gene editing system (e.g., CRISPR/Cas system or any of the gene editing systems described herein) is used to facilitate the insertion of a polynucleotide encoding PD-L1, into a genomic locus of the cell.

In some embodiments, PD-L1 protein expression is detected using a Western blot of cell lysates probed with antibodies against the PD-L1 protein. In another embodiment, reverse transcriptase polymerase chain reactions (RT-PCR) are used to confirm the presence of the exogenous PD-L1 mRNA.

In some embodiments, the engineered islets contains an exogenous polynucleotide that encodes FasL, such as human FasL. In some embodiments, FasL is overexpressed in the cell. In some embodiments, the expression of FasL is increased in the engineered islets compared to a similar reference or unmodified cell (including with any other modifications, such as genetic modifications) except that the reference or unmodified cell does not include the exogenous polynucleotide encoding FasL. Useful genomic, polynucleotide and polypeptide information about human Fas ligand (which is known as FasL, FASLG, CD178, TNFSF6, and the like) are provided in, for example, the GeneCard Identifier GC01P172628, HGNC No. 11936, NCBI Gene ID 356, Uniprot No. P48023, and NCBI RefSeq Nos. NP_000630.1, NM 000639.2, NP_001289675.1, and NM_001302746.1. In certain embodiments, the polynucleotide encoding Fas-L is operably linked to a promoter.

In some embodiments, the polynucleotide encoding Fas-L is inserted into any one of the gene loci described herein. In some cases, the polynucleotide encoding Fas-L is inserted into a safe harbor locus, such as but not limited to, a gene locus selected from AAVS1, CCR5, CLYBL, ROSA26, and SHS231. In particular embodiments, the polynucleotide encoding Fas-L is inserted into the CCR5 gene locus, the PPPIR12C (also known as AAVS1) gene locus or the CLYBL gene locus. In some embodiments, the polynucleotide encoding Fas-L is inserted into a B2M gene locus or a CIITA gene locus. In some embodiments, a suitable gene editing system (e.g., CRISPR/Cas system or any of the gene editing systems described herein) is used to facilitate the insertion of a polynucleotide encoding Fas-L, into a genomic locus of the cell.

In some embodiments, Fas-L protein expression is detected using a Western blot of cell lysates probed with antibodies against the Fas-L protein. In another embodiment, reverse transcriptase polymerase chain reactions (RT-PCR) are used to confirm the presence of the exogenous Fas-L mRNA.

In some embodiments, the engineered islets contains an exogenous polynucleotide that encodes CCL21, such as human CCL21. In some embodiments, CCL21 is overexpressed in the cell. In some embodiments, the expression of CCL21 is increased in the engineered islets compared to a similar reference or unmodified cell (including with any other modifications, such as genetic modifications) except that the reference or unmodified cell does not include the exogenous polynucleotide encoding CCL21. Useful genomic, polynucleotide and polypeptide information about human CCL21 are provided in, for example, the GeneCard Identifier GC09M034709, HGNC No. 10620, NCBI Gene ID 6366, Uniprot No. 000585, and NCBI RefSeq Nos. NP_002980.1 and NM_002989.3. In certain embodiments, the polynucleotide encoding CCL21 is operably linked to a promoter.

In some embodiments, the polynucleotide encoding CCL21 is inserted into any one of the gene loci described herein. In some cases, the polynucleotide encoding CCL21 is inserted into a safe harbor locus, such as but not limited to, a gene locus selected from AAVS1, CCR5, CLYBL, ROSA26, and SHS231. In particular embodiments, the polynucleotide encoding CCL21 is inserted into the CCR5 gene locus, the PPP1R12C (also known as AAVS1) gene locus or the CLYBL gene locus. In some embodiments, the polynucleotide encoding CCL21 is inserted into a B2M gene locus, a CIITA gene locus. In some embodiments, a suitable gene editing system (e.g., CRISPR/Cas system or any of the gene editing systems described herein) is used to facilitate the insertion of a polynucleotide encoding CCL21, into a genomic locus of the cell.

In some embodiments, CCL21 protein expression is detected using a Western blot of cell lysates probed with antibodies against the CCL21 protein. In another embodiment, reverse transcriptase polymerase chain reactions (RT-PCR) are used to confirm the presence of the exogenous CCL21 mRNA.

In some embodiments, the engineered islets contains an exogenous polynucleotide that encodes CCL22, such as human CCL22. In some embodiments, CCL22 is overexpressed in the cell. In some embodiments, the expression of CCL22 is increased in the engineered islets compared to a similar reference or unmodified cell (including with any other modifications, such as genetic modifications) except that the reference or unmodified cell does not include the exogenous polynucleotide encoding CCL22. Useful genomic, polynucleotide and polypeptide information about human CCL22 are provided in, for example, the GeneCard Identifier GC16P057359, HGNC No. 10621, NCBI Gene ID 6367, Uniprot No. 000626, and NCBI RefSeq Nos. NP_002981.2, NM_002990.4, XP_016879020.1, and XM_017023531.1. In certain embodiments, the polynucleotide encoding CCL22 is operably linked to a promoter.

In some embodiments, the polynucleotide encoding CCL22 is inserted into any one of the gene loci described herein. In some cases, the polynucleotide encoding CCL22 is inserted into a safe harbor locus, such as but not limited to, a gene locus selected from AAVS1, CCR5, CLYBL, ROSA26, and SHS231. In particular embodiments, the polynucleotide encoding CCL22 is inserted into the CCR5 gene locus, the PPP1R12C (also known as AAVS1) gene locus or the CLYBL gene locus. In some embodiments, the polynucleotide encoding CCL22 is inserted into a B2M gene locus, a CIITA gene locus. In some embodiments, a suitable gene editing system (e.g., CRISPR/Cas system or any of the gene editing systems described herein) is used to facilitate the insertion of a polynucleotide encoding CCL22, into a genomic locus of the cell.

In some embodiments, CCL22 protein expression is detected using a Western blot of cell lysates probed with antibodies against the CCL22 protein. In another embodiment, reverse transcriptase polymerase chain reactions (RT-PCR) are used to confirm the presence of the exogenous CCL22 mRNA.

In some embodiments, the engineered islets contains an exogenous polynucleotide that encodes Mfge8, such as human Mfge8. In some embodiments, Mfge8 is overexpressed in the cell. In some embodiments, the expression of Mfge8 is increased in the engineered islets compared to a similar reference or unmodified cell (including with any other modifications, such as genetic modifications) except that the reference or unmodified cell does not include the exogenous polynucleotide encoding Mfge8. Useful genomic, polynucleotide and polypeptide information about human Mfge8 are provided in, for example, the GeneCard Identifier GC15M088898, HGNC No. 7036, NCBI Gene ID 4240, Uniprot No. Q08431, and NCBI RefSeq Nos. NP_001108086.1, NM_001114614.2, NP_001297248.1, NM_001310319. 1, NP_001297249.1, NM_001310320.1, NP 001297250.1, NM_001310321.1, NP_005919.2, and NM_005928.3. In certain embodiments, the polynucleotide encoding Mfge8 is operably linked to a promoter.

In some embodiments, the polynucleotide encoding Mfge8 is inserted into any one of the gene loci described herein. In some cases, the polynucleotide encoding Mfge8 is inserted into a safe harbor locus, such as but not limited to, a gene locus selected from AAVS1, CCR5, CLYBL, ROSA26, and SHS231. In particular embodiments, the polynucleotide encoding Mfge8 is inserted into the CCR5 gene locus, the PPP1R12C (also known as AAVS1) gene locus or the CLYBL gene locus. In some embodiments, the polynucleotide encoding Mfge8 is inserted into a B2M gene locus, a CIITA gene locus. In some embodiments, a suitable gene editing system (e.g., CRISPR/Cas system or any of the gene editing systems described herein) is used to facilitate the insertion of a polynucleotide encoding Mfge8, into a genomic locus of the cell.

In some embodiments, Mfge8 protein expression is detected using a Western blot of cell lysates probed with antibodies against the Mfge8 protein. In another embodiment, reverse transcriptase polymerase chain reactions (RT-PCR) are used to confirm the presence of the exogenous Mfge8 mRNA.

In some embodiments, the engineered islets contains an exogenous polynucleotide that encodes SerpinB9, such as human SerpinB9. In some embodiments, SerpinB9 is overexpressed in the cell. In some embodiments, the expression of SerpinB9 is increased in the engineered islets compared to a similar reference or unmodified cell (including with any other modifications, such as genetic modifications) except that the reference or unmodified cell does not include the exogenous polynucleotide encoding SerpinB9. Useful genomic, polynucleotide and polypeptide information about human SerpinB9 are provided in, for example, the GeneCard Identifier GC06M002887, HGNC No. 8955, NCBI Gene ID 5272, Uniprot No. P50453, and NCBI RefSeq Nos. NP_004146.1, NM_004155.5, XP_005249241.1, and XM_005249184.4. In certain embodiments, the polynucleotide encoding SerpinB9 is operably linked to a promoter.

In some embodiments, the polynucleotide encoding SerpinB9 is inserted into any one of the gene loci described herein. In some cases, the polynucleotide encoding SerpinB9 is inserted into a safe harbor locus, such as but not limited to, a gene locus selected from AAVS1, CCR5, CLYBL, ROSA26, and SHS231. In particular embodiments, the polynucleotide encoding SerpinB9 is inserted into the CCR5 gene locus, the PPP1R12C (also known as AAVS1) gene locus or the CLYBL gene locus. In some embodiments, the polynucleotide encoding SerpinB9 is inserted into a B2M gene locus, a CIITA gene locus. In some embodiments, a suitable gene editing system (e.g., CRISPR/Cas system or any of the gene editing systems described herein) is used to facilitate the insertion of a polynucleotide encoding SerpinB9, into a genomic locus of the cell.

In some embodiments, SerpinB9 protein expression is detected using a Western blot of cell lysates probed with antibodies against the SerpinB9 protein. In another embodiment, reverse transcriptase polymerase chain reactions (RT-PCR) are used to confirm the presence of the exogenous SerpinB9 mRNA.

In some embodiments, the tolerogenic factor overexpressed or increased in the cell, e.g. engineered hypoimmunogenic islets, is an engineered CD47 protein. In some embodiments, the engineered CD47 protein have fewer amino acids than the wild-type full-length human CD47 protein. Such engineered proteins afford more efficient cell engineering approaches, including delivery via integrating gene therapy vectors. In some embodiments, the engineered CD47 proteins overexpressed or increased in the engineered hypoimmunogenic islets, are those described in PCT Publication No. WO2023158836, which is hereby incorporated by reference in its entirety.

CD47, also known as integrin-associated protein (IAP) or MER6, is a transmembrane protein that, in humans, is encoded by the human CD47 gene. CD47 is a member of the immunoglobulin (Ig) superfamily and is involved in a range of cellular processes, including apoptosis, proliferation, adhesion, and migration.

Human CD47 has a single IgV-like domain at its N-terminus, a highly hydrophobic stretch with five membrane-spanning segments, and an alternatively spliced cytoplasmic tail at its C-terminus. In addition, it has two extracellular regions and two intracellular regions between neighboring membrane-spanning segments. The signal peptide, when it exists on a CD47 isoform, is located at the N-terminus of the IgV-like domain.

As used herein, a human CD47 extracellular domain refers to the IgV-like domain at the N-terminus of the human CD47 protein. Structurally, the human CD47 extracellular domain is the N-terminal portion of the human CD47 protein that is located outside a cell when the human CD47 protein is anchored in the cell membrane. In some embodiments, the human CD47 extracellular domain has an amino acid sequence corresponding to amino acids 19-141 of SEQ ID NO:78, or an amino acid sequence that has at least 80%, 85%, 90%, 91%, 92%, 93%, 94%, 95%, 96%, 97%, 98%, or 99% identity with amino acids 19-141 of SEQ ID NO: 78. In some embodiments, the human CD47 extracellular domain has an amino acid sequence corresponding to amino acids 19-141 of SEQ ID NO: 78, or an amino acid sequence that has at least about 80%, 85%, 90%, 91%, 92%, 93%, 94%, 95%, 96%, 97%, 98%, or 99% identity with amino acids 19-141 of SEQ ID NO: 78.

As used herein, a human CD47 intracellular domain refers to the cytoplasmic tail at the C-terminus of the human CD47 protein. Structurally, the human CD47 intracellular domain is the C-terminal portion of the human CD47 protein that is located inside a cell when the human CD47 protein is anchored in the cell membrane. The human CD47 intracellular domain is alternatively spliced in vivo. In some embodiments, the human CD47 intracellular domain has an amino acid sequence corresponding to amino acids 290-323 of SEQ ID NO: 78, or an amino acid sequence that has at least 80%, 85%, 90%, 91%, 92%, 93%, 94%, 95%, 96%, 97%, 98%, or 99% identity with amino acids 290-323 of SEQ ID NO: 78. In some embodiments, the human CD47 intracellular domain has an amino acid sequence corresponding to amino acids 290-323 of SEQ ID NO: 78, or an amino acid sequence that has at least about 80%, 85%, 90%, 91%, 92%, 93%, 94%, 95%, 96%, 97%, 98%, or 99% identity with amino acids 290-323 of SEQ ID NO: 78.

As used herein, a human CD47 transmembrane domain refers to one of the membrane-spanning segments of the human CD47 protein. In some embodiments, the human CD47 transmembrane domain has an amino acid sequence corresponding to amino acids 142-162, 177-197, 208-228, 236-257, or 269-289 of SEQ ID NO: 78, or an amino acid sequence that has at least 80%, 85%, 90%, 91%, 92%, 93%, 94%, 95%, 96%, 97%, 98%, or 99% identity with amino acids 142-162, 177-197, 208-228, 236-257, or 269-289 of SEQ ID NO: 78. In some embodiments, the human CD47 transmembrane domain has an amino acid sequence corresponding to amino acids 142-162, 177-197, 208-228, 236-257, or 269-289 of SEQ ID NO: 78, or an amino acid sequence that has at least about 80%, 85%, 90%, 91%, 92%, 93%, 94%, 95%, 96%, 97%, 98%, or 99% identity with amino acids 142-162, 177-197, 208-228, 236-257, or 269-289 of SEQ ID NO: 78.

As used herein, a signal peptide refers to the short peptide present at the N-terminus of the CD47 protein when the protein is initially translated. Signal peptides are usually cleaved off from a protein by a signal peptidase during or immediately after insertion into a cell membrane. Signal peptides function to prompt a cell to translocate the protein, usually to the plasma membrane. In some embodiments, the signal peptide for a human CD47 protein has an amino acid sequence corresponding to amino acids 1-18 of SEQ ID NO: 78, or an amino acid sequence that has at least 80%, 85%, 90%, 91%, 92%, 93%, 94%, 95%, 96%, 97%, 98%, or 99% identity with amino acids 1-18 of SEQ ID NO: 78. In some embodiments, the signal peptide for a human CD47 protein has an amino acid sequence corresponding to amino acids 1-18 of SEQ ID NO: 78, or an amino acid sequence that has at least about 80%, 85%, 90%, 91%, 92%, 93%, 94%, 95%, 96%, 97%, 98%, or 99% identity with amino acids 1-18 of SEQ ID NO: 78.

The wild-type full-length human CD47 protein, as used herein, refers to the isoform CD47-202 as disclosed in the Ensembl database as of the filing date of this patent application. The wild-type full-length human CD47 protein has an amino acid sequence of SEQ ID NO: 78, wherein amino acids 1-18 are the signal peptide, amino acids 19-141 are the extracellular domain, amino acids 142-162, 177-197, 208-228, 236-257, 269-289 are the five transmembrane domains, and amino acids 290-323 are the intracellular domain. Amino acids 163-176 and 229-235 are the two intracellular connections between the transmembrane domains, and amino acids 198-207 and 257-268 are the two extracellular connections between the transmembrane domains.

In some embodiments, the engineered CD47 protein is a C-terminally truncated version of isoform 202 (SEQ ID NO: 78). For example, in some embodiments, the C-terminal truncation is consecutive and is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, or 161 amino acid(s) long. In some embodiments, the engineered CD47 protein having a C-terminal truncation of SEQ ID NO: 78 further has an N-terminal truncation of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, or 140 consecutive amino acid(s).

In some embodiments, the engineered CD47 protein is a C-terminally truncated version of isoform 201 (SEQ ID NO: 77). For example, in some embodiments, the C-terminal truncation is consecutive and is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, or 143 amino acid(s) long. In some embodiments, the engineered CD47 protein having a C-terminal truncation of SEQ ID NO: 77 further has an N-terminal truncation of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95 consecutive amino acid(s).

In some embodiments, the engineered CD47 protein is a C-terminally truncated version of isoform 206 (SEQ ID NO: 79). For example, in some embodiments, the C-terminal truncation is consecutive and is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, or 66 amino acid(s) long. In some embodiments, the engineered CD47 protein having a C-terminal truncation of SEQ ID NO: 79 further has an N-terminal truncation of 1, 2,3, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, or 140 consecutive amino acid(s).

In some embodiments, the engineered CD47 protein comprises a minimal intracellular domain. As used herein, a minimal intracellular domain refers to an intracellular domain that has the minimum number of amino acids required to preserve SIRPα binding of the engineered CD47 protein.

In some embodiments, the engineered CD47 protein comprises a minimal extracellular domain. As used herein, a minimal extracellular domain refers to an extracellular domain that has the minimum number of amino acids required for the engineered CD47 protein to bind to SIRPα.

In an aspect, the present disclosure provides an engineered CD47 protein that comprises a human CD47 extracellular domain or a portion thereof and at least one human CD47 transmembrane domain, wherein when an intracellular domain exists, it is a human CD47 intracellular domain with a deletion of at least one amino acid. In some embodiments, when there are more than one transmembrane domains in the engineered CD47 protein, each of the transmembrane domains are interconnected with intracellular and/or extracellular connection(s).

In an aspect, the present disclosure provides an engineered CD47 protein that consists essentially of a human CD47 extracellular domain or a portion thereof and at least one human CD47 transmembrane domain. In some embodiments, when there are more than one transmembrane domain in the engineered CD47 protein, each of the transmembrane domains are interconnected with intracellular and/or extracellular connection(s). As used herein, the term “consisting essentially of” includes the specified elements and any additional elements that do not abrogate SIRPα binding of the engineered CD47 protein.

In an aspect, the present disclosure provides an engineered CD47 protein that consists of a human CD47 extracellular domain or a portion thereof and at least one human CD47 transmembrane domain. In some embodiments, when there are more than one transmembrane domain in the engineered CD47 protein, each of the transmembrane domains are interconnected with intracellular and/or extracellular connection(s).

In some embodiments, the engineered CD47 protein comprises a human CD47 extracellular domain or a portion thereof, at least one human CD47 transmembrane domain or a portion thereof, and a portion of a human CD47 intracellular domain comprising a deletion of at least one amino acid, wherein the deletion is not a C-terminal deletion of 18 amino acids.

In some embodiments, the engineered CD47 protein comprises a portion of a human CD47 extracellular domain, at least one human CD47 transmembrane domain or a portion thereof, and a portion of a human CD47 intracellular domain comprising a C-terminal deletion of 18 amino acids.

In some embodiments, the engineered CD47 protein comprises a human CD47 extracellular domain or a portion thereof, at least one and fewer than five human CD47 transmembrane domain(s) or portion(s) thereof, and a portion of a human CD47 intracellular domain comprising a C-terminal deletion of 18 amino acids.

In some embodiments, the engineered CD47 protein comprises a human CD47 extracellular domain or a portion thereof, at least one human CD47 transmembrane domain or a portion thereof, and a signal peptide, wherein the engineered CD47 protein does not comprise an intracellular domain.

In some embodiments, the engineered CD47 protein comprises a human CD47 extracellular domain, and at least one human CD47 transmembrane domain or a portion thereof, wherein the engineered CD47 protein does not comprise an intracellular domain.

In some embodiments, the engineered CD47 protein comprises a human CD47 extracellular domain or a portion thereof, and at least one and fewer than five human CD47 transmembrane domain(s) or portion(s) thereof, wherein the engineered CD47 protein does not comprise an intracellular domain.

In some embodiments, the engineered CD47 protein comprises a human CD47 extracellular domain, five human CD47 transmembrane domains and a human CD47 intracellular domain with a C-terminal deletion of 18 amino acids, wherein the amino acid sequence of the engineered CD47 protein is not SEQ ID NO: 77.

In some embodiments, the engineered CD47 protein comprises a human CD47 extracellular domain and five human CD47 transmembrane domain, wherein the amino acid sequence of the engineered CD47 protein is not SEQ ID NO: 79.

In some embodiments, the engineered CD47 protein comprises a human CD47 extracellular domain or a portion thereof, at least one human CD47 transmembrane domain or a portion thereof, and no intracellular domain or a human CD47 intracellular domain comprising a deletion of at least one amino acid, wherein the amino acid sequence of the engineered CD47 protein has at most 99% identity with SEQ ID NO: 77 and SEQ ID NO: 79. In other words, in some embodiments, the engineered CD47 protein comprises a human CD47 extracellular domain or a portion thereof, at least one human CD47 transmembrane domain or a portion thereof, and no intracellular domain or a human CD47 intracellular domain comprising a deletion of at least one amino acid, wherein the amino acid sequence of the engineered CD47 protein has 99% identity or less to SEQ ID NO: 77 and has 99% identity or less to SEQ ID NO: 79.

In some embodiments, the human CD47 extracellular domain in the engineered CD47 protein is a wild-type human CD47 extracellular domain. In some embodiments, the wild-type domain has an amino acid sequence corresponding to amino acids 19-141 of SEQ ID NO: 77, or to amino acids 1-96 of SEQ ID NO: 79.

In some embodiments, the human CD47 extracellular domain in the engineered CD47 protein has an amino acid sequence with at least 80%, 85%, 90%, 91%, 92%, 93%, 94%, 95%, 96%, 97%, 98%, 99%, or 100% identity to amino acids 19-141 of SEQ ID NO: 77, or to amino acids 1-96 of SEQ ID NO: 79. In some embodiments, the human CD47 extracellular domain in the engineered CD47 protein has an amino acid sequence with at least about 80%, 85%, 90%, 91%, 92%, 93%, 94%, 95%, 96%, 97%, 98%, 99%, or 100% identity to amino acids 19-141 of SEQ ID NO: 77, or to amino acids 1-96 of SEQ ID NO: 79.

In some embodiments, the human CD47 extracellular domain in the engineered CD47 protein is structurally equivalent to a wild-type human CD47 extracellular domain.

As used herein, “structurally equivalent” refers to two amino acid sequences having at least 80%, 85%, 90%, 91%, 92%, 93%, 94%, 95%, 96%, 97%, 98%, 99%, or 100% identity. Preferably, the sequence variation does not change the engineered CD47 protein's biological activity. In some embodiments, the sequence variation does not prevent the engineered human protein from binding to SIRPα. In some embodiments, the sequence variation does not prevent the engineered human protein from being a tolerogenic factor. In some embodiments, at least a portion of the sequence variation may occur through conservative amino acid substitution(s).

In some embodiments, an engineered protein of the present disclosure comprises one or more membrane tethers. In some embodiments, one or more membrane tethers are or comprise a transmembrane domain. In some embodiments, a transmembrane domain comprises a GPCR transmembrane domain selected from the group consisting of: 5-hydroxytryptamine (serotonin) receptor 1A (HTR1A), 5-hydroxytryptamine (serotonin) receptor 1B (HTR1B), 5-hydroxytryptamine (serotonin) receptor 1D (HTR1D), 5-hydroxytryptamine (serotonin) receptor IE (HTR1E), 5-hydroxytryptamine (serotonin) receptor 1F (HTR1F), 5-hydroxytryptamine (serotonin) receptor 2A (HTR2A), 5-hydroxytryptamine (serotonin) receptor 2B (HTR2B), 5-hydroxytryptamine (serotonin) receptor 2C (HTR2C), 5-hydroxytryptamine (serotonin) receptor 4 (HTR4), 5-hydroxytryptamine (serotonin) receptor 5A (HTR5A), 5-hydroxytryptamine (serotonin) receptor 5B (HTR5BP), 5-hydroxytryptamine (serotonin) receptor 6 (HTR6), 5-hydroxytryptamine (serotonin) receptor 7, adenylate cyclase-coupled (HTR7), cholinergic receptor, muscarinic 1 (CHRM1), cholinergic receptor, muscarinic 2 (CHRM2), cholinergic receptor, muscarinic 3 (CHRM3), cholinergic receptor, muscarinic 4 (CHRM4), cholinergic receptor, muscarinic 5 (CHRM5), adenosine A1 receptor (ADORA1), adenosine A2a receptor (ADORA2A), adenosine A2b receptor (ADORA2B), adenosine A3 receptor (ADORA3), adhesion G protein-coupled receptor A1 (ADGRA1), adhesion G protein-coupled receptor A2 (ADGRA2), adhesion G protein-coupled receptor A3 (ADGRA3), adhesion G protein-coupled receptor B1 (ADGRB1), adhesion G protein-coupled receptor B2 (ADGRB2), adhesion G protein-coupled receptor B3 (ADGRB3), cadherin EGF LAG seven-pass G-type receptor 1 (CELSR1), cadherin EGF LAG seven-pass G-type receptor 2 (CELSR2), cadherin EGF LAG seven-pass G-type receptor 3 (CELSR3), adhesion G protein-coupled receptor D1 (ADGRD1), adhesion G protein-coupled receptor D2 (ADGRD2), adhesion G protein-coupled receptor E1 (ADGRE1), adhesion G protein-coupled receptor E2 (ADGRE2), adhesion G protein-coupled receptor E3 (ADGRE3), adhesion G protein-coupled receptor E4 (ADGRE4P), adhesion G protein-coupled receptor E5 (ADGRE5), adhesion G protein-coupled receptor F1 (ADGRF1), adhesion G protein-coupled receptor F2 (ADGRF2), adhesion G protein-coupled receptor F3 (ADGRF3), adhesion G protein-coupled receptor F4 (ADGRF4), adhesion G protein-coupled receptor F5 (ADGRF5), adhesion G protein-coupled receptor G1 (ADGRG1), adhesion G protein-coupled receptor G2 (ADGRG2), adhesion G protein-coupled receptor G3 (ADGRG3), adhesion G protein-coupled receptor G4 (ADGRG4), adhesion G protein-coupled receptor G5 (ADGRG5), adhesion G protein-coupled receptor G6 (ADGRG6), adhesion G protein-coupled receptor G7 (ADGRG7), adhesion G protein-coupled receptor L1 (ADGRL1), adhesion G protein-coupled receptor L2 (ADGRL2), adhesion G protein-coupled receptor L3 (ADGRL3), adhesion G protein-coupled receptor L4 (ADGRL4), adhesion G protein-coupled receptor V1 (ADGRV1), adrenoceptor alpha 1A (ADRA1A), adrenoceptor alpha 1B (ADRA1B), adrenoceptor alpha 1D (ADRA1D), adrenoceptor alpha 2A (ADRA2A), adrenoceptor alpha 2B (ADRA2B), adrenoceptor alpha 2C (ADRA2C), adrenoceptor beta 1 (ADRB1), adrenoceptor beta 2 (ADRB2), adrenoceptor beta 3 (ADRB3), angiotensin II receptor type 1 (AGTR1), angiotensin II receptor type 2 (AGTR2), apelin receptor (APLNR), G protein-coupled bile acid receptor 1 (GPBAR1), neuromedin B receptor (NMBR), gastrin releasing peptide receptor (GRPR), bombesin like receptor 3 (BRS3), bradykinin receptor B1 (BDKRB1), bradykinin receptor B2 (BDKRB2), calcitonin receptor (CALCR), calcitonin receptor like receptor (CALCRL), calcium sensing receptor (CASR), G protein-coupled receptor, class C (GPRC6A), cannabinoid receptor 1 (brain) (CNR1), cannabinoid receptor 2 (CNR2), chemerin chemokine-like receptor 1 (CMKLR1), chemokine (C-C motif) receptor 1 (CCR1), chemokine (C-C motif) receptor 2 (CCR2), chemokine (C-C motif) receptor 3 (CCR3), chemokine (C-C motif) receptor 4 (CCR4), chemokine (C-C motif) receptor 5 (gene/pseudogene) (CCR5), chemokine (C-C motif) receptor 6 (CCR6), chemokine (C-C motif) receptor 7 (CCR7), chemokine (C-C motif) receptor 8 (CCR8), chemokine (C-C motif) receptor 9 (CCR9), chemokine (C-C motif) receptor 10 (CCR10), chemokine (C-X-C motif) receptor 1 (CXCR1), chemokine (C-X-C motif) receptor 2 (CXCR2), chemokine (C-X-C motif) receptor 3 (CXCR3), chemokine (C-X-C motif) receptor 4 (CXCR4), chemokine (C-X-C motif) receptor 5 (CXCR5), chemokine (C-X-C motif) receptor 6 (CXCR6), chemokine (C-X3-C motif) receptor 1 (CX3CR1), chemokine (C motif) receptor 1 (XCR1), atypical chemokine receptor 1 (Duffy blood group) (ACKR1), atypical chemokine receptor 2 (ACKR2), atypical chemokine receptor 3 (ACKR3), atypical chemokine receptor 4 (ACKR4), chemokine (C-C motif) receptor-like 2 (CCRL2), cholecystokinin A receptor (CCKAR), cholecystokinin B receptor (CCKBR), G protein-coupled receptor 1 (GPR1), bombesin like receptor 3 (BRS3), G protein-coupled receptor 3 (GPR3), G protein-coupled receptor 4 (GPR4), G protein-coupled receptor 6 (GPR6), G protein-coupled receptor 12 (GPR12), G protein-coupled receptor 15 (GPR15), G protein-coupled receptor 17 (GPR17), G protein-coupled receptor 18 (GPR18), G protein-coupled receptor 19 (GPR19), G protein-coupled receptor 20 (GPR20), G protein-coupled receptor 21 (GPR21), G protein-coupled receptor 22 (GPR22), G protein-coupled receptor 25 (GPR25), G protein-coupled receptor 26 (GPR26), G protein-coupled receptor 27 (GPR27), G protein-coupled receptor 31 (GPR31), G protein-coupled receptor 32 (GPR32), G protein-coupled receptor 33 (gene/pseudogene) (GPR33), G protein-coupled receptor 34 (GPR34), G protein-coupled receptor 35 (GPR35), G protein-coupled receptor 37 (endothelin receptor type B-like) (GPR37), G protein-coupled receptor 37 like 1 (GPR37L1), G protein-coupled receptor 39 (GPR39), G protein-coupled receptor 42 (gene/pseudogene) (GPR42), G protein-coupled receptor 45 (GPR45), G protein-coupled receptor 50 (GPR50), G protein-coupled receptor 52 (GPR52), G protein-coupled receptor 55 (GPR55), G protein-coupled receptor 61 (GPR61), G protein-coupled receptor 62 (GPR62), G protein-coupled receptor 63 (GPR63), G protein-coupled receptor 65 (GPR65), G protein-coupled receptor 68 (GPR68), G protein-coupled receptor 75 (GPR75), G protein-coupled receptor 78 (GPR78), G protein-coupled receptor 79 (GPR79), G protein-coupled receptor 82 (GPR82), G protein-coupled receptor 83 (GPR83), G protein-coupled receptor 84 (GPR84), G protein-coupled receptor 85 (GPR85), G protein-coupled receptor 87 (GPR87), G protein-coupled receptor 88 (GPR88), G protein-coupled receptor 101 (GPR101), G protein-coupled receptor 119 (GPR119), G protein-coupled receptor 132 (GPR132), G protein-coupled receptor 135 (GPR135), G protein-coupled receptor 139 (GPR139), G protein-coupled receptor 141 (GPR141), G protein-coupled receptor 142 (GPR142), G protein-coupled receptor 146 (GPR146), G protein-coupled receptor 148 (GPR148), G protein-coupled receptor 149 (GPR149), G protein-coupled receptor 150 (GPR150), G protein-coupled receptor 151 (GPR151), G protein-coupled receptor 152 (GPR152), G protein-coupled receptor 153 (GPR153), G protein-coupled receptor 160 (GPR160), G protein-coupled receptor 161 (GPR161), G protein-coupled receptor 162 (GPR162), G protein-coupled receptor 171 (GPR171), G protein-coupled receptor 173 (GPR173), G protein-coupled receptor 174 (GPR174), G protein-coupled receptor 176 (GPR176), G protein-coupled receptor 182 (GPR182), G protein-coupled receptor 183 (GPR183), leucine-rich repeat containing G protein-coupled receptor 4 (LGR4), leucine-rich repeat containing G protein-coupled receptor 5 (LGR5), leucine-rich repeat containing G protein-coupled receptor 6 (LGR6), MAS1 proto-oncogene (MAS1), MAS1 proto-oncogene like (MAS1L), MAS related GPR family member D (MRGPRD), MAS related GPR family member E (MRGPRE), MAS related GPR family member F (MRGPRF), MAS related GPR family member G (MRGPRG), MAS related GPR family member X1 (MRGPRX1), MAS related GPR family member X2 (MRGPRX2), MAS related GPR family member X3 (MRGPRX3), MAS related GPR family member X4 (MRGPRX4), opsin 3 (OPN3), opsin 4 (OPN4), opsin 5 (OPN5), purinergic receptor P2Y (P2RY8), purinergic receptor P2Y (P2RY10), trace amine associated receptor 2 (TAAR2), trace amine associated receptor 3 (gene/pseudogene) (TAAR3), trace amine associated receptor 4 (TAAR4P), trace amine associated receptor 5 (TAAR5), trace amine associated receptor 6 (TAAR6), trace amine associated receptor 8 (TAAR8), trace amine associated receptor 9 (gene/pseudogene) (TAAR9), G protein-coupled receptor 156 (GPR156), G protein-coupled receptor 158 (GPR158), G protein-coupled receptor 179 (GPR179), G protein-coupled receptor, class C (GPRC5A), G protein-coupled receptor, class C (GPRC5B), G protein-coupled receptor, class C (GPRC5C), G protein-coupled receptor, class C (GPRC5D), frizzled class receptor 1 (FZD1), frizzled class receptor 2 (FZD2), frizzled class receptor 3 (FZD3), frizzled class receptor 4 (FZD4), frizzled class receptor 5 (FZD5), frizzled class receptor 6 (FZD6), frizzled class receptor 7 (FZD7), frizzled class receptor 8 (FZD8), frizzled class receptor 9 (FZD9), frizzled class receptor 10 (FZD10), smoothened, frizzled class receptor (SMO), complement component 3a receptor 1 (C3AR1), complement component 5a receptor 1 (C5AR1), complement component 5a receptor 2 (C5AR2), corticotropin releasing hormone receptor 1 (CRHR1), corticotropin releasing hormone receptor 2 (CRHR2), dopamine receptor D1 (DRD1), dopamine receptor D2 (DRD2), dopamine receptor D3 (DRD3), dopamine receptor D4 (DRD4), dopamine receptor D5 (DRD5), endothelin receptor type A (EDNRA), endothelin receptor type B (EDNRB), formyl peptide receptor 1 (FPR1), formyl peptide receptor 2 (FPR2), formyl peptide receptor 3 (FPR3), free fatty acid receptor 1 (FFAR1), free fatty acid receptor 2 (FFAR2), free fatty acid receptor 3 (FFAR3), free fatty acid receptor 4 (FFAR4), G protein-coupled receptor 42 (gene/pseudogene) (GPR42), gamma-aminobutyric acid (GABA) B receptor, 1 (GABBR1), gamma-aminobutyric acid (GABA) B receptor, 2 (GABBR2), galanin receptor 1 (GALR1), galanin receptor 2 (GALR2), galanin receptor 3 (GALR3), growth hormone secretagogue receptor (GHSR), growth hormone releasing hormone receptor (GHRHR), gastric inhibitory polypeptide receptor (GIPR), glucagon like peptide 1 receptor (GLP1R), glucagon-like peptide 2 receptor (GLP2R), glucagon receptor (GCGR), secretin receptor (SCTR), follicle stimulating hormone receptor (FSHR), luteinizing hormone/choriogonadotropin receptor (LHCGR), thyroid stimulating hormone receptor (TSHR), gonadotropin releasing hormone receptor (GNRHR), gonadotropin releasing hormone receptor 2 (pseudogene) (GNRHR2), G protein-coupled receptor 18 (GPR18), G protein-coupled receptor 55 (GPR55), G protein-coupled receptor 119 (GPR119), G protein-coupled estrogen receptor 1 (GPER1), histamine receptor H1 (HRH1), histamine receptor H2 (HRH2), histamine receptor H3 (HRH3), histamine receptor H4 (HRH4), hydroxycarboxylic acid receptor 1 (HCAR1), hydroxycarboxylic acid receptor 2 (HCAR2), hydroxycarboxylic acid receptor 3 (HCAR3), KISS1 receptor (KISSIR), leukotriene B4 receptor (LTB4R), leukotriene B4 receptor 2 (LTB4R2), cysteinyl leukotriene receptor 1 (CYSLTR1), cysteinyl leukotriene receptor 2 (CYSLTR2), oxoeicosanoid (OXE) receptor 1 (OXERI), formyl peptide receptor 2 (FPR2), lysophosphatidic acid receptor 1 (LPAR1), lysophosphatidic acid receptor 2 (LPAR2), lysophosphatidic acid receptor 3 (LPAR3), lysophosphatidic acid receptor 4 (LPAR4), lysophosphatidic acid receptor 5 (LPAR5), lysophosphatidic acid receptor 6 (LPAR6), sphingosine-1-phosphate receptor 1 (S1PR1), sphingosine-1-phosphate receptor 2 (S1PR2), sphingosine-1-phosphate receptor 3 (S1PR3), sphingosine-1-phosphate receptor 4 (S1PR4), sphingosine-1-phosphate receptor 5 (S1PR5), melanin concentrating hormone receptor 1 (MCHR1), melanin concentrating hormone receptor 2 (MCHR2), melanocortin 1 receptor (alpha melanocyte stimulating hormone receptor) (MCIR), melanocortin 2 receptor (adrenocorticotropic hormone) (MC2R), melanocortin 3 receptor (MC3R), melanocortin 4 receptor (MC4R), melanocortin 5 receptor (MC5R), melatonin receptor 1A (MTNR1A), melatonin receptor 1B (MTNR1B), glutamate receptor, metabotropic 1 (GRM1), glutamate receptor, metabotropic 2 (GRM2), glutamate receptor, metabotropic 3 (GRM3), glutamate receptor, metabotropic 4 (GRM4), glutamate receptor, metabotropic 5 (GRM5), glutamate receptor, metabotropic 6 (GRM6), glutamate receptor, metabotropic 7 (GRM7), glutamate receptor, metabotropic 8 (GRM8), motilin receptor (MLNR), neuromedin U receptor 1 (NMUR1), neuromedin U receptor 2 (NMUR2), neuropeptide FF receptor 1 (NPFFR1), neuropeptide FF receptor 2 (NPFFR2), neuropeptide S receptor 1 (NPSR1), neuropeptides B/W receptor 1 (NPBWR1), neuropeptides B/W receptor 2 (NPBWR2), neuropeptide Y receptor Y1 (NPY1R), neuropeptide Y receptor Y2 (NPY2R), neuropeptide Y receptor Y4 (NPY4R), neuropeptide Y receptor Y5 (NPY5R), neuropeptide Y receptor Y6 (pseudogene) (NPY6R), neurotensin receptor 1 (high affinity) (NTSR1), neurotensin receptor 2 (NTSR2), opioid receptor, delta 1 (OPRD1), opioid receptor, kappa 1 (OPRK1), opioid receptor, mu 1 (OPRM1), opiate receptor-like 1 (OPRL1), hypocretin (orexin) receptor 1 (HCRTR1), hypocretin (orexin) receptor 2 (HCRTR2), G protein-coupled receptor 10⁷ (GPR10⁷), G protein-coupled receptor 137 (GPR137), olfactory receptor family 51 subfamily E member 1 (OR51E1), transmembrane protein, adipocyte associated 1 (TPRA1), G protein-coupled receptor 143 (GPR143), G protein-coupled receptor 157 (GPR157), oxoglutarate (alpha-ketoglutarate) receptor 1 (OXGR1), purinergic receptor P2Y (P2RY 1), purinergic receptor P2Y (P2RY2), pyrimidinergic receptor P2Y (P2RY4), pyrimidinergic receptor P2Y (P2RY6), purinergic receptor P2Y (P2RY11), purinergic receptor P2Y (P2RY12), purinergic receptor P2Y (P2RY13), purinergic receptor P2Y (P2RY14), parathyroid hormone 1 receptor (PTH1R), parathyroid hormone 2 receptor (PTH2R), platelet-activating factor receptor (PTAFR), prokineticin receptor 1 (PROKR1), prokineticin receptor 2 (PROKR2), prolactin releasing hormone receptor (PRLHR), prostaglandin D2 receptor (DP) (PTGDR), prostaglandin D2 receptor 2 (PTGDR2), prostaglandin E receptor 1 (PTGER1), prostaglandin E receptor 2 (PTGER2), prostaglandin E receptor 3 (PTGER3), prostaglandin E receptor 4 (PTGER4), prostaglandin F receptor (PTGFR), prostaglandin 12 (prostacyclin) receptor (IP) (PTGIR), thromboxane A2 receptor (TBXA2R), coagulation factor II thrombin receptor (F2R), F2R like trypsin receptor 1 (F2RL1), coagulation factor II thrombin receptor like 2 (F2RL2), F2R like thrombin/trypsin receptor 3 (F2RL3), pyroglutamylated RFamide peptide receptor (QRFPR), relaxin/insulin-like family peptide receptor 1 (RXFP1), relaxin/insulin-like family peptide receptor 2 (RXFP2), relaxin/insulin-like family peptide receptor 3 (RXFP3), relaxin/insulin-like family peptide receptor 4 (RXFP4), somatostatin receptor 1 (SSTR1), somatostatin receptor 2 (SSTR2), somatostatin receptor 3 (SSTR3), somatostatin receptor 4 (SSTR4), somatostatin receptor 5 (SSTR5), succinate receptor 1 (SUCNR1), tachykinin receptor 1 (TACRI), tachykinin receptor 2 (TACR2), tachykinin receptor 3 (TACR3), taste 1 receptor member 1 (TAS1R1), taste 1 receptor member 2 (TAS1R2), taste 1 receptor member 3 (TAS1R3), taste 2 receptor member 1 (TAS2R1), taste 2 receptor member 3 (TAS2R3), taste 2 receptor member 4 (TAS2R4), taste 2 receptor member 5 (TAS2R5), taste 2 receptor member 7 (TAS2R7), taste 2 receptor member 8 (TAS2R8), taste 2 receptor member 9 (TAS2R9), taste 2 receptor member 10 (TAS2R10), taste 2 receptor member 13 (TAS2R13), taste 2 receptor member 14 (TAS2R14), taste 2 receptor member 16 (TAS2R16), taste 2 receptor member 19 (TAS2R19), taste 2 receptor member 20 (TAS2R20), taste 2 receptor member 30 (TAS2R30), taste 2 receptor member 31 (TAS2R31), taste 2 receptor member 38 (TAS2R38), taste 2 receptor member 39 (TAS2R39), taste 2 receptor member 40 (TAS2R40), taste 2 receptor member 41 (TAS2R41), taste 2 receptor member 42 (TAS2R42), taste 2 receptor member 43 (TAS2R43), taste 2 receptor member 45 (TAS2R45), taste 2 receptor member 46 (TAS2R46), taste 2 receptor member 50 (TAS2R50), taste 2 receptor member 60 (TAS2R60), thyrotropin-releasing hormone receptor (TRHR), trace amine associated receptor 1 (TAAR1), urotensin 2 receptor (UTS2R), arginine vasopressin receptor 1A (AVPR1A), arginine vasopressin receptor 1B (AVPR1B), arginine vasopressin receptor 2 (AVPR2), oxytocin receptor (OXTR), adenylate cyclase activating polypeptide 1 (pituitary) receptor type I (ADCYAPIRI), vasoactive intestinal peptide receptor 1 (VIPR1), vasoactive intestinal peptide receptor 2 (VIPR2), and any variant thereof. In some embodiments, a transmembrane domain is or comprises a CD3zeta, CD8a, CD16a, CD28, CD32a, CD32c, CD40, CD47, CD64, ICOS, Dectin-1, DNGR1, EGFR, GPCR, MyD88, PDGFR, SLAMF7, TRL1, TLR2, TLR3, TRL4, TLR5, TLR6, TLR7, TLR8, TLR9, or VEGFR transmembrane domain.

Plasma membrane proteins can be attached to the peripheral membrane or can be integral membrane proteins. See, for example, a review in Komath S S, Fujita M, Hart G W, et al.

Glycosylphosphatidylinositol Anchors. In: Varki A, Cummings R D, Esko J D, et al., editors. Essentials of Glycobiology. 4th edition. Cold Spring Harbor (NY): Cold Spring Harbor Laboratory Press; 2022. Chapter 12. GPI anchorage refers to the attachment of glycosylphosphatidylinositol, or GPI, to the C-terminus of a protein during posttranslational modification. In some embodiments, a heterologous membrane attachment sequence is a GPI anchor attachment sequence. Proteins that are attached to GPI anchors via their C-terminus are typically found in the outer lipid bilayer. GPI anchors are alternatives to the single transmembrane domain of type-I integral membrane proteins.

A heterologous GPI anchor attachment sequence can be derived from any known GPI-anchored protein (reviewed in Ferguson M A J, Kinoshita T, Hart G W. Glycosylphosphatidylinositol Anchors. In: Varki A, Cummings R D, Esko J D, et al., editors. Essentials of Glycobiology. 2nd edition. Cold Spring Harbor (NY): Cold Spring Harbor Laboratory Press; 2009. Chapter 11). In some embodiments, a heterologous GPI anchor attachment sequence is a GPI anchor attachment sequence from CD14, CD16, CD48, DAF/CD55, CD59, CD80, CD87, or TRAIL-R3. In some embodiments, a heterologous GPI anchor attachment sequence is derived from DAF/CD55. In some embodiments, a heterologous GPI anchor attachment sequence is derived from CD59. In some embodiments, a heterologous GPI anchor attachment sequence is derived from TRAIL-R3. In illustrative embodiments, a heterologous GPI anchor attachment sequence is derived from DAF/CD55, CD59, or TRAIL-R3. In some embodiments, one or both of the activation elements include a heterologous signal sequence to help direct expression of the activation element to the cell membrane. Any signal sequence that is active in the packaging cell line can be used. In some embodiments, a signal sequence is a DAF/CD55 signal sequence. In some embodiments, a signal sequence is a CD59 signal sequence. In some embodiments, a signal sequence is a TRAIL-R3 signal sequence.

In some embodiments, the engineered CD47 protein comprises one or more wild-type human CD47 transmembrane domains. In some embodiments, the wild-type domain has an amino acid sequence corresponding to amino acids 142-162, 177-197, 208-228, 236-257, or 269-289 of SEQ ID NO: 78.

In some embodiments, the engineered CD47 protein comprises one or more transmembrane domains that are structurally equivalent to a wild-type human CD47 transmembrane domain. In some embodiments, the engineered CD47 protein comprises one or more transmembrane domains having an amino acid sequence with at least 80%, 90%, 95%, 96%, 97%, 98%, 99%, or 100% identity to amino acids 142-162, 177-197, 208-228, 236-257, or 269-289 of SEQ ID NO: 78.

In some embodiments, the human CD47 intracellular domain in the engineered CD47 protein is a wild-type human CD47 intracellular domain. In some embodiments, the wild-type intracellular domain has an amino acid sequence corresponding to amino acids 290-323 of SEQ ID NO: 78.

In some embodiments, the human CD47 intracellular domain in the engineered CD47 protein is structurally equivalent to a wild-type human CD47 intracellular domain. In some embodiments, the human CD47 intracellular domain in the engineered CD47 protein has an amino acid sequence with at least 80%, 90%, 95%, 96%, 97%, 98%, 99%, or 100% identity to amino acids 290-323 of SEQ ID NO: 78.

In some embodiments, the engineered CD47 protein is a transmembrane protein. A transmembrane protein is an integral membrane protein that spans the entirety of the cell membrane and has both intracellular and extracellular portions. As used herein, “intracellular portion” can include the CD47 intracellular domain and the intracellular connections, when present in the molecule. As used herein, “extracellular portion” can include the CD47 extracellular domain and the extracellular connections, when present in the molecule.

As used herein, a wild-type human CD47 extracellular domain refers to the extracellular domain of any one of the wild-type human CD47 protein isoforms. A wild-type human CD47 transmembrane domain refers to a transmembrane domain of any one of the wild-type human CD47 protein isoforms. A wild-type human CD47 intracellular domain refers to the intracellular domain of any one of the wild-type human CD47 protein isoforms.

In some embodiments, the engineered CD47 protein is an engineered human CD47 protein, an engineered humanized CD47 protein, or an engineered partially-humanized CD47 protein.

As used herein, “humanized” or “humanization” means that the amino acid sequence of the engineered CD47 protein is modified to reduce its immunogenicity in humans. As used herein, “partially-humanized” or “partial humanization” means that a portion of the amino acid sequence of the engineered CD47 protein is modified to reduce the engineered CD47 protein's immunogenicity in humans. For example, in some embodiments, the extracellular domain of the engineered CD47 protein is modified to reduce the engineered CD47 protein's immunogenicity in humans. Humanization is usually achieved by modifying a protein sequence from a non-human source to increase its similarity to its counterpart protein produced naturally in humans. Two major approaches have been used to humanize proteins: rational design and empirical methods. The rational design methods are characterized by protein structural modeling, generating a few variants of the protein and assessing their binding or any other property of interest. In contrast to the rational design methods, empirical methods do not require the structure information of the protein. They depend on the generation of large combinatorial libraries and selection of the desired variants by enrichment technologies such as phage, ribosome or yeast display, or by high throughput screening techniques. These methods rest on selection rather than making assumptions on the impact of mutations on the protein structure. For example, in some embodiments, humanization of the engineered CD47 protein comprises grafting the SIRPα binding region in the engineered CD47 protein onto a human CD47 protein. In other embodiments, humanization of the engineered CD47 protein comprises introducing one or more point mutations in the engineered CD47 protein so that one or more residue(s) in the engineered CD47 protein is substituted with the corresponding residue in a human CD47 protein.

In some embodiments, an engineered protein of the present disclosure comprises a SIRPα interaction motif comprising a SIRPα antibody. In some embodiments, a SIRPα antibody is selected from Table 3.

TABLE 3
SIRPα Antibodies
Table 3. SIRPα Antibodies

| Name | Associated Patent | Non-Patent Lit | Company/Institution |
|---|---|---|---|
| ADU-1805 | 2020 US20220135671 | 2019 Functional | Aduro |
| | 2018 US20180312587 | characterization of the | |
| | 2018 U.S. Pat. No. 10,851,164 | selective pan-allele anti- | |
| | | SIRPα antibody ADU-1805 | |
| | | that blocks the SIRPα-CD47 | |
| | | innate immune checkpoint. | |
| Alector patent | 2022 US20220289844 | | Alector |
| anti-SIRPA | 2019 US20210277113 | | |
| | 2019 US20190359707 | | |
| | 2019 WO2019226973 | | |
| | 2019 U.S. Pat. No. 11,319,373 | | |
| | 2017 WO2018107058 | | |
| | 2017 US20190275150 | | |
| ALX Oncology | 2022 US20220242928 | | ALX Oncology |
| patent anti- | 2022 US20220324996 | | |
| SIRPa | 2021 US20210363269 | | |
| | 2021 US20220002434 | | |
| | 2021 U.S. Pat. No. 11,242,404 | | |
| | 2021 US20230018821 | | |
| | 2021 WO2021247430 | | |
| | 2020 US20200392199 | | |
| | 2020 US20210070838 | | |
| | 2020 U.S. Pat. No. 11,208,459 | | |
| | 2019 US20190352419 | | |
| | 2019 U.S. Pat. No. 11,292,850 | | |
| | 2017 US20180105600 | | |
| | 2017 U.S. Pat. No. 11,401,338 | | |
| Apexigen | 2021 WO2021174127 | | Apexigen |
| patent anti- | | | |
| SIRPA | | | |
| Arch Oncology | 2021 US20220226469 | 2021 Novel SIRPα | Arch Oncology |
| patent anti- | 2021 WO2021222746 | Antibodies That Induce | |
| SIRPA | 2019 WO2020102422 | Single-Agent Phagocytosis of | |
| | 2019 US20200297842 | Tumor Cells while Preserving | |
| | 2019 U.S. Pat. No. 11,202,828 | T Cells. | |
| BI 765063 | 2022 WO2022254379 | 2022 NCT05249426 Phase 1 | Boehringer |
| | 2022 US20230012273 | A Study to Test Whether | OSE |
| | 2022 US20220281991 | Different Combinations of BI | Immunotherapeutics |
| | 2019 WO2019175218 | 765063, Ezabenlimab, | |
| | 2018 WO2019073080 | Chemotherapy, Cetuximab, | |
| | 2018 US20210179728 | and BI 836880 Help People | |
| | 2017 WO2017178653 | With Head and Neck Cancer | |
| | 2017 US20190127477 | or Liver Cancer | |
| | 2017 U.S. Pat. No. 11,279,766 | 2020 NCT04653142 Phase 1 | |
| | | A Study to Test Different | |
| | | Doses of BI 765063 Alone | |
| | | and in Combination With BI | |
| | | 754091 in Japanese Patients | |
| | | With Different Types of | |
| | | Advanced Cancer (Solid | |
| | | Tumors) | |
| Bio-Thera | 2022 WO2022247933 | | Bio-Thera Solutions |
| patent anti- | | | |
| SIRPA | | | |
| Bioray Pharma | 2021 WO2022121980 | | Bioray Pharma |
| patent anti- | | | Zheijang Hisun |
| SIRPA | | | |
| Byondis patent | 2022 US20220195063 | | Byondis |
| anti-SIRPA | 2019 US20210388107 | | |
| | 2018 US20210070874 | | |
| | 2018 U.S. Pat. No. 11,274,159 | | |
| CC-95251 | 2019 WO2020068752 | 2022 NCT05168202 Phase 1 | Celgene |
| | | A Study to Assess the Effect | |
| | | of CC-95251 in Participants | |
| | | With Acute Myeloid | |
| | | Leukemia and | |
| | | Myelodysplastic Syndromes | |
| | | 2019 NCT03783403 Phase 1 | |
| | | A Study of CC-95251, a | |
| | | Monoclonal Antibody | |
| | | Directed Against SIRPα, | |
| | | With Cetuximab in Subjects | |
| | | With Advanced Solid | |
| | | Cancers | |
| | | 2019 NCT03816254 Phase 1 | |
| | | A Study of CC-95251, a | |
| | | Monoclonal Antibody | |
| | | Directed Against SIRPα, | |
| | | With Cetuximab in Subjects | |
| | | With Advanced Solid | |
| | | Cancers | |
| ES004 | 2020 WO2021032078 | | Elpiscience |
| | 2020 US20210347908 | | |
| Hospital for | 2012 WO2013056352 | | Toronto Hospital for |
| Sick Children | 2012 US20140242095 | | Sick Children |
| patent anti- | | | |
| SIRPA | | | |
| Kobe U. anti- | 2021 WO2022102634 | 2022 Anticancer efficacy of | Daiichi Sankyo |
| SIRPA | 2019 WO2020013170 | monotherapy with antibodies | Kobe University |
| | 2019 US20210155707 | to SIRPα/SIRPβ1 mediated | |
| | | by induction of | |
| | | antitumorigenic macrophages. | |
| | | 2018 Anti-human SIRPα | |
| | | antibody is a new tool for | |
| | | cancer immunotherapy. | |
| L & L | 2021 WO2021185273 | | L&L Biopharma |
| Biopharma | | | |
| patent anti- | | | |
| SIRPalpha | | | |
| Lanova patent | 2021 US20220169726 | | Lanova |
| anti-SIRPA | 2020 WO2021129697 | | |
| Qure patent | 2021 WO2022110922 | | QureBio |
| anti-SIRPA | | | |
| Sairopa patent | 2022 US20220135677 | | Sairopa |
| anti-SIRPA | | | |
| Sound | 2020 WO2020180811 | | Sound Biologics |
| Biologics | 2020 US20220235144 | | |
| patent anti- | | | |
| SIRPA | | | |
| Stanford U. | 2021 US20210171654 | 2017 Anti-SIRPα antibody | Forty Seven |
| patent anti- | 2020 WO2021011912 | immunotherapy enhances | Stanford |
| SIRPA | 2020 US20200262918 | neutrophil and macrophage | |
| | 2018 U.S. Pat. No. 10,487,150 | antitumor activity. | |
| | 2018 WO2019023347 | | |
| | 2018 US20190119396 | | |
| | 2018 U.S. Pat. No. 10,961,318 | | |
| | 2018 US20180319883 | | |
| | 2018 U.S. Pat. No. 10,781,256 | | |
| | 2017 US20180037652 | | |
| | 2017 U.S. Pat. No. 10,611,842 | | |
| | 2016 WO2016205042 | | |
| | 2016 US20180171030 | | |
| | 2016 U.S. Pat. No. 10,618,976 | | |
| | 2015 WO2015138600 | | |
| | 2015 US20170073414 | | |
| | 2015 U.S. Pat. No. 10,081,680 | | |
| Synthon patent | 2018 WO2018210795 | | Synthon |
| anti-SIRPA | 2018 WO2018210793 | | |
| Electra patent | 2021 WO2021226576 | | Electra |
| anti- | 2021 WO2021226591 | | |
| SIRPA/SIRPB | | | |
| Forty Seven | 2020 WO2020198353 | | Fort Seven |
| patent anti- | | | |
| SIRPA/ | | | |
| SIGLEC-10 | | | |
| IMM2902 | | 2022 NCT05076591 Phase 1 | ImmuneOnco |
| | | IMM2902, a HER2/SIRPα | |
| | | Bispecific mAb-Trap | |
| | | Antibody-receptor Fusion | |
| | | Protein, in Patients With | |
| | | HER2-expressing Advanced | |
| | | Solid Tumors | |
| Lanova patent | 2021 WO2022127901 | | Lanova |
| anti-SIRPA/ | | | |
| PD-L1 | | | |
| OSE patent | 2019 US20220056135 | | OSE |
| anti-PD-1/ | | | Immunotherapeutics |
| SIRPA | | | |

In some embodiments, the SIRPα antibody or a portion thereof comprises an amino acid sequence that is at least 80% identical to SEQ ID NO: 64. In some embodiments, the SIRPα antibody or a portion thereof comprises an amino acid sequence that is at least 80% identical to SEQ ID NO: 65. In some embodiments, the SIRPα antibody or a portion thereof comprises an amino acid sequence that is at least 80% identical to SEQ ID NO: 66. In some embodiments, the SIRPα antibody or a portion thereof comprises an amino acid sequence that is at least 80% identical to SEQ ID NO: 67. In some embodiments, the SIRPα antibody or a portion thereof comprises an amino acid sequence that is at least 80% identical to SEQ ID NO: 68. In some embodiments, the SIRPα antibody or a portion thereof comprises an amino acid sequence that is at least 80% identical to SEQ ID NO: 69. In some embodiments, the SIRPα antibody or a portion thereof comprises an amino acid sequence that is at least 80% identical to SEQ ID NO: 70. In some embodiments, the SIRPα antibody or a portion thereof comprises an amino acid sequence that is at least 80% identical to SEQ ID NO: 71. In some embodiments, the SIRPα antibody or a portion thereof comprises an amino acid sequence that is at least 80% identical to SEQ ID NO: 72. In some embodiments, the SIRPα antibody or a portion thereof comprises an amino acid sequence that is at least 80% identical to SEQ ID NO: 73. In some embodiments, the SIRPα antibody or a portion thereof comprises an amino acid sequence that is at least 80% identical to SEQ ID NO: 74. In some embodiments, the SIRPα antibody or a portion thereof comprises an amino acid sequence that is at least 80% identical to SEQ ID NO: 75. In some embodiments, the SIRPα antibody or a portion thereof comprises an amino acid sequence that is at least 80% identical to SEQ ID NO: 76.

The human CD47 protein is glycosylated. Protein glycosylation involves the covalent attachment of glycans (also called carbohydrates, saccharides, or sugars) to a protein. Based on the amino acid side-chain atoms to which glycans are linked, most protein glycosylations fall within two categories: N-linked glycosylation and O-linked glycosylation. In N-linked glycosylation, glycans are attached to the side-chain nitrogen atoms of asparagine residues in a conserved consensus sequence Asn-Xaa-Ser/Thr (Xaa≠Pro), whereas in O-linked glycosylation, glycans are attached to the side-chain oxygen atoms of hydroxyl amino acids, primarily serine and threonine residues.

The IgV domain of wild type human CD47 protein is N-glycosylated and modified with O-linked glycosaminoglycans. The human CD47 protein can be expressed as a proteoglycan with a molecular weight of >250 kDa, having both heparan and chondroitin sulfate glycosaminoglycan (GAG) chains at Ser⁶⁴ and Ser⁷⁹ (Kaur et al., J. Biological Chemistry, 2011). Heparan sulfate (HSGAG) and chondroitin sulfate (CSGAG) are synthesized in the Golgi apparatus, where protein cores made in the rough endoplasmic reticulum are post-translationally modified with O-linked glycosylation by glycosyltransferases forming proteoglycans. In addition, N-linked glycosylation has been identified at four of the five potential modification sites (N¹⁶, N³², N⁵⁵, and N⁹³) in the human CD47 protein (Hatherley et al., Cell, 2008). The numbering of amino acid in this paragraph is based on SEQ ID NO: 2 (i.e., mature, full length, wild type CD47).

In some embodiments, the engineered CD47 protein comprises fewer glycosylation modification sites than a wild-type human CD47 protein. A glycosylation modification site refers to a sequence of consecutive amino acids in a protein that can serve as the attachment site for a glycan. Glycosylation modification sites are also called sequons. In some embodiments, one or more amino acid(s) within the glycosaminoglycan modification site of a wild-type human CD47 protein is deleted or substituted in the engineered CD47 protein. In some embodiments, the engineered CD47 protein has 0, 1, 2, 3, 4, 5, or 6 glycosylation site(s).

In some embodiments, the engineered CD47 protein comprises fewer glycosylation modifications than a wild-type human CD47 protein. The glycosylation modifications include, but are not limited to, N-glycosylation, O glycosylation, phosphoserine glycosylation, and C-glycosylation. In some embodiments, the engineered CD47 protein has 0, 1, 2, 3, 4, or 5 glycosylation modification(s).

In some embodiments, the engineered CD47 protein comprises fewer glycosaminoglycan modification sites than a wild-type human CD47 protein. A glycosaminoglycan modification site refers to a sequence of consecutive amino acids in a protein that can serve as the attachment site for a glycosaminoglycan. In some embodiments, one or more amino acid(s) within the glycosaminoglycan modification site of a wild-type human CD47 protein is deleted or substituted in the engineered CD47 protein. In some embodiments, the engineered CD47 protein has 0 or 1 glycosaminoglycan modification site.

In some embodiments, the engineered CD47 protein comprises fewer glycosaminoglycan chains than a wild-type human CD47 protein. Glycosaminoglycan chains include, but are not limited to, heparan sulfate (HSGAG), chondroitin sulfate (CSGAG), keratan sulfate, and hyaluronic acid. In some embodiments, the engineered CD47 protein has 0 or 1 glycosaminoglycan side chain.

In some embodiments, the engineered CD47 protein comprises fewer than two heparan and/or chondroitin sulfate glycosaminoglycan modification sites. In some embodiments, one or more amino acid(s) within the glycosaminoglycan modification sites of a wild-type human CD47 protein is deleted or substituted in the engineered CD47 protein. In some embodiments, the engineered CD47 protein comprises one heparan and/or chondroitin sulfate glycosaminoglycan modification site. In some embodiments, the engineered CD47 protein comprises no heparan and/or chondroitin sulfate glycosaminoglycan modification sites.

In some embodiments, the engineered CD47 protein comprises fewer than two heparan and/or chondroitin sulfate glycosaminoglycan chains. In some embodiments, the engineered CD47 protein has 0 or 1 heparan and/or chondroitin sulfate glycosaminoglycan chains.

In some embodiments, the engineered CD47 protein comprises fewer N-linked glycosylation sites than a wild-type human CD47 protein. An N-linked glycosylation site is a sequence of consecutive amino acids in a protein that can serve as the attachment site for a saccharide, particularly an N-glycan. In some embodiments, the engineered CD47 protein has 0, 1, 2, 3, or 4 N-linked glycosylation site(s).

In some embodiments, the engineered CD47 protein comprises fewer N-linked glycosylation modifications than a wild-type human CD47 protein. In some embodiments, the engineered CD47 protein has 0, 1, 2, or 3 N-linked glycosylation modification(s).

a) Interaction with SIRP proteins

The signal regulator protein (SIRP) family contains three members, and of these SIRPα and SIRPγ are known CD47 receptors. SIRP proteins belong to the Ig family of cell surface glycoproteins, and the first member identified was SIRPα (also known as SHPS-1, CD172a, BIT, MFR, or P84). SIRPα is highly expressed in myeloid cells and neurons, but also in endothelial cells and fibroblasts, and has three extracellular Ig-like domains, one distal IgV-like domain, and two membrane proximal IgC-like domains. In addition, an alternatively spliced form having only one IgV domain has also been reported. In its intracellular tail, SIRPα has two immunoreceptor tyrosine-based inhibitory motifs (ITIMs), which when phosphorylated, can bind the Src homology 2 (SH2) domain-containing protein-tyrosine phosphatases SHP-1 and SHP-2. Additional cytoplasmic binding partners for SIRPα are the adaptor molecules Src kinase-associated protein of 55 kDa homolog/SKAP2 (SKAP55hom/R), Fyn-binding protein/SLP-76-associated phosphoprotein of 130 kDa (FYB/SLAP-130), and the tyrosine kinase PYK2. SIRPα is also a substrate for the kinase activity of the insulin, EGF, and bPDGF receptors. The overexpression of SIRPα in fibroblasts decreases proliferation and other downstream events in response to insulin, EGF, and bPDGF. Since SIRPα is also constitutively associated with the M-CSF receptor c-fms, SIRPα overexpression partially reverses the v-fins phenotype.

CD47 is a ligand for SIRPα. The glycosylation of CD47 or SIRPα does not seem to be necessary for their interaction, but the level of N-glycosylation of SIRPα has an impact on the interaction, such that over glycosylation reduces the binding of CD47. The long-range disulfide bond between Cys³³ in the CD47 IgV domain and Cys²⁶³ in the CD47 transmembrane domain is also important to establish an orientation of the CD47 IgV domain that enhances its binding to SIRPα. The numbering of amino acids is based on SEQ ID NO:2 in this paragraph (i.e., the mature, wild-type, full-length CD47 protein).

The structure of CD47 and the CD47/SIRPα complex have been revealed (Hatherley et al., 2008). Two P-strands, corresponding to amino acids 92-100 and 103-113 of SEQ ID NO:2, were identified to be at the core of the CD47/SIRPα interaction. Amino acids 97 (Glu), 99 (Thr), 100 (Glu), 103 (Arg), 104 (Glu), and 106 (Glu) of SEQ ID NO:2 were identified as contact residues in the CD47/SIRPα interaction and play a role in CD47's binding affinity for SIRPα (Hatherley et al., 2008). In preferred embodiments, the two P-strands and the six contact residues within them are retained in the engineered CD47 proteins disclosed herein to maintain SIRPα binding capability.

In some embodiments, the engineered CD47 protein comprises at least one SIRPα interaction motif in its extracellular domain. In some embodiments, the amino acids corresponding to amino acids 97, 99, 100, 103, 104, 106 of SEQ ID NO:2 are retained in the engineered CD47 protein. In some embodiments, the two P-strands are retained in the engineered CD47 protein.

In some embodiments, the engineered CD47 protein comprises a disulfide bond between a cysteine within the human CD47 extracellular domain or portion thereof and a cysteine within or between the human CD47 transmembrane domain(s). In some embodiments, the two cysteines are Cys³³ in the extracellular domain and the Cys²⁶³ in the transmembrane domain, wherein the numbering is based on SEQ ID NO:2.

In some embodiments, the engineered CD47 protein can bind to SIRPα. In some embodiments, the engineered CD47 protein can bind to SIRPα with a binding affinity that is similar to a wild-type human CD47 protein. In some embodiments, the engineered CD47 protein binds to SIRPα with a K_{D} lower than about 0.01 μM, 0.02 μM, 0.03 μM, 0.04 μM, 0.05 μM, 0.06 μM, 0.07 μM, 0.08 μM, 0.09 M, 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 M, 5 μM. In some embodiments, the engineered CD47 protein can bind to SIRPα with a higher binding affinity than a wild-type human CD47 protein.

Direct binding studies demonstrate that TSP-1 inhibits SIRPα binding to cells expressing CD47 (Isenberg et al., 2009). This data is consistent with competitive binding of TSP-1 and SIRPα to a single site on CD47, steric inhibition of binding to distinct but proximal sites, or allosteric inhibition. Glycosylation at Ser⁶⁴ with a heparan sulfate chain is necessary for TSP-1 binding but not SIRPα binding (Soto-Pantoja et al., 2015). Therefore, in some embodiments, the engineered CD47 protein that lacks glycosylation at Ser⁶⁴ has enhanced SIRPα binding capacity because TSP-1 binding is hindered.

The CD47/SIRPα interaction regulates a multitude of intercellular interactions in many body systems, such as the immune system where it regulates lymphocyte homeostasis, dendritic cell (DC) maturation and activation, proper localization of certain DC subsets in secondary lymphoid organs, and cellular transmigration. The CD47/SIRPα interaction also regulates cells of the nervous system. An interaction between these two proteins also plays an important role in bone remodeling. Cellular responses regulated by the CD47/SIRPα interaction are often dependent on a bidirectional signaling through both receptors.

CD47 on host cells can function as a “marker of self” and regulate phagocytosis by binding to SIRPα on the surface of circulating immune cells to deliver an inhibitory “don't kill me” signal. As disclosed above, SIRPα encodes an Ig-superfamily receptor expressed on the surface of macrophages and dendritic cells, whose cytoplasmic region contains immunoreceptor tyrosine-based inhibition motifs (ITIMs) that can trigger a cascade to inhibit phagocytosis. CD47-SIRPα binding results in phosphorylation of ITIMs on SIRPα, which triggers recruitment of the SHP1 and SHP2 Src homology phosphatases. These phosphatases, in turn, inhibit accumulation of myosin II at the phagocytic synapse, preventing phagocytosis (Fujioka et al., 1996). Phagocytosis of target cells by macrophages is ultimately regulated by a balance of activating signals (FcγR, CRT, LRP-1) and inhibitory signals (SIRPα-CD47). Elevated expression of CD47 can help the cell evade immune surveillance and subsequent destruction. Elevated expression of CD47 can help the cell evade innate immune cell killing.

In some embodiments, the engineered CD47 protein is a tolerogenic factor. As used herein, tolerogenic factor is an agent that induces immune tolerance when there is pathological or undesirable activation of the normal immune response. This can occur, for example, when a patient develops an immune reaction to donor antigens after receiving an allogeneic transplantation or an allogeneic cell therapy, or when the body responds inappropriately to self-antigens implicated in autoimmune diseases. In some embodiments, “tolerogenic factor” includes hypoimmunity factors, complement inhibitors, and other factors that modulate or affect the ability of a cell to be recognized by the immune system of a host or recipient subject upon administration, transplantation, or engraftment. In some embodiments, the tolerogenic factor is genetically modified to achieve additional functions.

In some embodiments, the engineered CD47 protein can inhibit phagocytosis, release of cytotoxic agents, and/or other mechanisms of cell-mediated killing.

### b) Interactions Between CD47 Molecules

It has also been shown that CD47 mediates cell adhesion interactions in the absence of any known CD47 ligands. This cell-cell adhesion, which requires CD47 but not any of its known ligands, suggests that homotypic binding can also occur between the IgV domains of CD47 on opposing cells (Rebres et al., 2005). This interaction may require an unidentified trypsin-sensitive protein (X) to mediate cell-cell adhesion, but the potential should be considered that this cell-cell interaction and homotypic binding of proteolytically shed CD47 IgV domain (Maile et al., 2010) or CD47 in exosomes (Kaur et al., 2014) to cell surface CD47 could elicit CD47 signal transduction. However, direct evidence for CD47-CD47 binding and signaling resulting from homotypic CD47 binding is lacking. In some embodiments, the engineered CD47 protein cannot interact with another CD47 molecule.

### 2. Methods of Increasing Expression (e.g., Overexpression) of a Polynucleotide

In some embodiments, increased expression of a polynucleotide may be carried out by any of a variety of techniques. For instance, methods for modulating expression of genes and factors (proteins) include genome editing technologies, and RNA or protein expression technologies and the like. For all of these technologies, well known recombinant techniques are used, to generate recombinant nucleic acids as outlined herein. In some embodiments, the engineered islets with the one or more modifications for overexpression or increased expression of a polynucleotide is any source cell as described herein. In some embodiments, the source cell is any cell described herein.

In some embodiments, expression of a gene is increased by increasing endogenous gene activity (e.g., increasing transcription of the exogenous gene). In some cases, endogenous gene activity is increased by increasing activity of a promoter or enhancer operably linked to the endogenous gene. In some embodiments, increasing activity of the promoter or enhancer comprises making one or more modifications to an endogenous promoter or enhancer that increase activity of the endogenous promoter or enhancer. In some cases, increasing gene activity of an endogenous gene comprises modifying an endogenous promoter of the gene. In some embodiments increasing gene activity of an endogenous gene comprises introducing a heterologous promoter. In some embodiments, the heterologous promoter is selected from the group consisting of a CAG promoter, cytomegalovirus (CMV) promoter, EF1a promoter, PGK promoter, adenovirus late promoter, vaccinia virus 7.5K promoter, SV40 promoter, tk promoter of HSV, mouse mammary tumor virus (MMTV) promoter, LTR promoter of HIV, promoter of moloney virus, Epstein Barr virus (EBV) promoter, Rous sarcoma virus (RSV) promoter, and UBC promoter.

a. DNA-Binding Fusion Proteins

In some embodiments, expression of a target gene (e.g., CD47, or another tolerogenic factor) is increased by expression of fusion protein or a protein complex containing (1) a site-specific binding domain specific for the endogenous CD47, or other gene and (2) a transcriptional activator.

In some embodiments, the regulatory factor is comprised of a site specific DNA-binding nucleic acid molecule, such as a guide RNA (gRNA). In some embodiments, the method is achieved by site specific DNA-binding targeted proteins, such as zinc finger proteins (ZFP) or fusion proteins containing ZFP, which are also known as zinc finger nucleases (ZFNs).

In some embodiments, the regulatory factor comprises a site-specific binding domain, such as using a DNA binding protein or DNA-binding nucleic acid, which specifically binds to or hybridizes to the gene at a targeted region. In some embodiments, the provided polynucleotides or polypeptides are coupled to or complexed with a site-specific nuclease, such as a modified nuclease. For example, in some embodiments, the administration is effected using a fusion comprising a DNA-targeting protein of a modified nuclease, such as a meganuclease or an RNA-guided nuclease such as a clustered regularly interspersed short palindromic nucleic acid (CRISPR)-Cas system, such as CRISPR-Cas9 system. In some embodiments, the nuclease is modified to lack nuclease activity. In some embodiments, the modified nuclease is a catalytically dead dCas9.

In some embodiments, the site specific binding domain may be derived from a nuclease. For example, the recognition sequences of homing endonucleases and meganucleases such as I-SceI, I-CeuI, PI-PspI, PI-Sce, I-SceIV, I-CsmI, I-PanI, I-SceII, I-PpoI, I-SceIII, I-Crel, I-TevI, I-TevII and I-TevIII. See also U.S. Pat. Nos. 5,420,032; 6,833,252; Belfort et al., (1997) Nucleic Acids Res. 25:3379-3388; Dujon et al., (1989) Gene 82:115-118; Perler et al, (1994) Nucleic Acids Res. 22, 1125-1127; Jasin (1996) Trends Genet. 12:224-228; Gimble et al., (1996) J. Mol. Biol. 263:163-180; Argast et al, (1998) J. Mol. Biol. 280:345-353 and the New England Biolabs catalogue. In addition, the DNA-binding specificity of homing endonucleases and meganucleases can be engineered to bind non-natural target sites. See, for example, Chevalier et al, (2002) Molec. Cell 10:895-905; Epinat et al, (2003) Nucleic Acids Res. 31:2952-2962; Ashworth et al, (2006) Nature 441:656-659; Paques et al, (2007) Current Gene Therapy 7:49-66; U.S. Patent Publication No. 2007/0117128.

Zinc finger, TALE, and CRISPR system binding domains can be “engineered” to bind to a predetermined nucleotide sequence, for example via engineering (altering one or more amino acids) of the recognition helix region of a naturally occurring zinc finger or TALE protein. Engineered DNA binding proteins (zinc fingers or TALEs) are proteins that are non-naturally occurring. Rational criteria for design include application of substitution rules and computerized algorithms for processing information in a database storing information of existing ZFP and/or TALE designs and binding data. See, for example, U.S. Pat. Nos. 6,140,081; 6,453,242; and 6,534,261; see also WO 98/53058; WO 98/53059; WO 98/53060; WO 02/016536 and WO 03/016496 and U.S. Publication No. 20110301073.

In some embodiments, the site-specific binding domain comprises one or more zinc-finger proteins (ZFPs) or domains thereof that bind to DNA in a sequence-specific manner. A ZFP or domain thereof is a protein or domain within a larger protein that binds DNA in a sequence-specific manner through one or more zinc fingers, regions of amino acid sequence within the binding domain whose structure is stabilized through coordination of a zinc ion.

Among the ZFPs are artificial ZFP domains targeting specific DNA sequences, typically 9-18 nucleotides long, generated by assembly of individual fingers. ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3 and 6) on a zinc finger recognition helix. Thus, in some embodiments, the ZFP or ZFP-containing molecule is non-naturally occurring, e.g., is engineered to bind to a target site of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties.

Many gene-specific engineered zinc fingers are available commercially. For example, Sangamo Biosciences (Richmond, CA, USA) has developed a platform (CompoZr) for zinc-finger construction in partnership with Sigma-Aldrich (St. Louis, MO, USA), allowing investigators to bypass zinc-finger construction and validation altogether, and provides specifically targeted zinc fingers for thousands of proteins (Gaj et al., Trends in Biotechnology, 2013, 31(7), 397-405). In some embodiments, commercially available zinc fingers are used or are custom designed.

In some embodiments, the site-specific binding domain comprises a naturally occurring or engineered (non-naturally occurring) transcription activator-like protein (TAL) DNA binding domain, such as in a transcription activator-like protein effector (TALE) protein, See, e.g., U.S. Patent Publication No. 20110301073, incorporated by reference in its entirety herein.

In some embodiments, the site-specific binding domain is derived from the CRISPR/Cas system. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system, or a “targeting sequence”), and/or other sequences and transcripts from a CRISPR locus.

In general, a guide sequence includes a targeting domain (e.g. targeting sequence) comprising a polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. In some examples, the targeting domain of the gRNA is complementary, e.g., at least 80, 85, 90, 95, 98 or 99% complementary, e.g., fully complementary, to the target sequence on the target nucleic acid.

In some embodiments, the gRNA may be any as described herein. In particular embodiments, the gRNA has a targeting sequence that is complementary to a target site of CD47, such as set forth in any one of SEQ ID NOS: 200784-231885 (Table 29, Appendix 22 of WO2016183041); HLA-E, such as set forth in any one of SEQ ID NOS: 189859-193183 (Table 19, Appendix 12 of WO2016183041); HLA-F, such as set forth in any one of SEQ ID NOS: 688808-699754 (Table 45, Appendix 38 of WO2016183041); HLA-G, such as set forth in any one of SEQ ID NOS: 188372-189858 (Table 18, Appendix 11 of WO2016183041); or PD-L1, such as set forth in any one of SEQ ID NOS: 193184-200783 (Table 21, Appendix 14 of WO2016183041).

In some embodiments, the target site is upstream of a transcription initiation site of the target gene. In some embodiments, the target site is adjacent to a transcription initiation site of the gene. In some embodiments, the target site is adjacent to an RNA polymerase pause site downstream of a transcription initiation site of the gene.

In some embodiments, the targeting domain is configured to target the promoter region of the target gene to promote transcription initiation, binding of one or more transcription enhancers or activators, and/or RNA polymerase. One or more gRNA can be used to target the promoter region of the gene. In some embodiments, one or more regions of the gene can be targeted. In certain aspects, the target sites are within 600 base pairs on either side of a transcription start site (TSS) of the gene.

It is within the level of a skilled artisan to design or identify a gRNA sequence (i.e. gRNA targeting sequence) that is or comprises a sequence targeting a gene, including the exon sequence and sequences of regulatory regions, including promoters and activators. A genome-wide gRNA database for CRISPR genome editing is publicly available, which contains exemplary single guide RNA (sgRNA) target sequences in constitutive exons of genes in the human genome or mouse genome (see e.g., genescript.com/gRNA-database.html; see also, Sanjana et al. (2014) Nat. Methods, 11:783-4; www.e-crisp.org/E-CRISP/; crispr.mit.edu/). In some embodiments, the gRNA sequence is or comprises a targeting sequence with minimal off-target binding to a non-target gene.

In some embodiments, the regulatory factor further comprises a functional domain, e.g., a transcriptional activator.

In some embodiments, the transcriptional activator is or contains one or more regulatory elements, such as one or more transcriptional control elements of a target gene, whereby a site-specific domain as provided above is recognized to drive expression of such gene. In some embodiments, the transcriptional activator drives expression of the target gene. In some cases, the transcriptional activator, can be or contain all or a portion of a heterologous transactivation domain. For example, in some embodiments, the transcriptional activator is selected from Herpes simplex-derived transactivation domain, Dnmt3a methyltransferase domain, p65, VP16, and VP64.

In some embodiments, the regulatory factor is a zinc finger transcription factor (ZF-TF). In some embodiments, the regulatory factor is VP64-p65-Rta (VPR).

In certain embodiments, the regulatory factor further comprises a transcriptional regulatory domain. Common domains include, e.g., transcription factor domains (activators, repressors, co-activators, co-repressors), silencers, oncogenes (e.g., myc, jun, fos, myb, max, mad, rel, ets, bcl, myb, mos family members etc.); DNA repair enzymes and their associated factors and modifiers; DNA rearrangement enzymes and their associated factors and modifiers; chromatin associated proteins and their modifiers (e.g. kinases, acetylases and deacetylases); and DNA modifying enzymes (e.g., methyltransferases such as members of the DNMT family (e.g., DNMT1, DNMT3A, DNMT3B, DNMT3L, etc., topoisomerases, helicases, ligases, kinases, phosphatases, polymerases, endonucleases) and their associated factors and modifiers. See, e.g., U.S. Publication No. 2013/0253040, incorporated by reference in its entirety herein.

Suitable domains for achieving activation include the HSV VP 16 activation domain (see, e.g., Hagmann et al, J. Virol. 71, 5952-5962 (1 97)) nuclear hormone receptors (see, e.g., Torchia et al., Curr. Opin. Cell. Biol. 10:373-383 (1998)); the p65 subunit of nuclear factor kappa B (Bitko & Bank, J. Virol. 72:5610-5618 (1998) and Doyle & Hunt, Neuroreport 8:2937-2942 (1997)); Liu et al., Cancer Gene Ther. 5:3-28 (1998)), or artificial chimeric functional domains such as VP64 (Beerli et al., (1998) Proc. Natl. Acad. Sci. USA 95:14623-33), and degron (Molinari et al., (1999) EMBO J. 18, 6439-6447). Additional exemplary activation domains include, Oct 1, Oct-2A, Spl, AP-2, and CTF1 (Seipel et al, EMBOJ. 11, 4961-4968 (1992) as well as p300, CBP, PCAF, SRC1 PvALF, AtHD2A and ERF-2. See, for example, Robyr et al, (2000) Mol. Endocrinol. 14:329-347; Collingwood et al, (1999) J. Mol. Endocrinol 23:255-275; Leo et al, (2000) Gene 245:1-11; Manteuffel-Cymborowska (1999) Acta Biochim. Pol. 46:77-89; McKenna et al, (1999) J. Steroid Biochem. Mol. Biol. 69:3-12; Malik et al, (2000) Trends Biochem. Sci. 25:277-283; and Lemon et al, (1999) Curr. Opin. Genet. Dev. 9:499-504. Additional exemplary activation domains include, but are not limited to, OsGAI, HALF-1, Cl, AP1, ARF-5, -6, -1, and -8, CPRF1, CPRF4, MYC-RP/GP, and TRAB1, See, for example, Ogawa et al, (2000) Gene 245:21-29; Okanami et al, (1996) Genes Cells 1:87-99; Goff et al, (1991) Genes Dev. 5:298-309; Cho et al, (1999) Plant Mol Biol 40:419-429; Ulmason et al, (1999) Proc. Natl. Acad. Sci. USA 96:5844-5849; Sprenger-Haussels et al, (2000) Plant J. 22:1-8; Gong et al, (1999) Plant Mol. Biol. 41:33-44; and Hobo et al., (1999) Proc. Natl. Acad. Sci. USA 96:15,348-15,353.

Exemplary repression domains that can be used to make genetic repressors include, but are not limited to, KRAB A/B, KOX, TGF-beta-inducible early gene (TIEG), v-erbA, SID, MBD2, MBD3, members of the DNMT family (e.g., DNMT1, DNMT3A, DNMT3B, DNMT3L, etc.), Rb, and MeCP2. See, for example, Bird et al, (1999) Cell 99:451-454; Tyler et al, (1999) Cell 99:443-446; Knoepfler et al, (1999) Cell 99:447-450; and Robertson et al, (2000) Nature Genet. 25:338-342. Additional exemplary repression domains include, but are not limited to, ROM2 and AtHD2A. See, for example, Chem et al, (1996) Plant Cell 8:305-321; and Wu et al, (2000) Plant J. 22:19-27.

In some instances, the domain is involved in epigenetic regulation of a chromosome. In some embodiments, the domain is a histone acetyltransferase (HAT), e.g. type—A, nuclear localized such as MYST family members MOZ, Ybf2/Sas3, MOF, and Tip60, GNAT family members Gcn5 or pCAF, the p300 family members CBP, p300 or Rtt109 (Bemdsen and Denu (2008) Curr Opin Struct Biol 18(6):682-689). In other instances, the domain is a histone deacetylase (HD AC) such as the class I (HDAC-1, 2, 3, and 8), class II molecules (HDAC IIA (HDAC-4, 5, 7 and 9), HD AC IIB (HDAC 6 and 10)), class IV (HDAC-1 1), class III (also known as sirtuins (SIRTs); SIRT1-7) (see Mottamal et al., (2015) Molecules 20(3):3898-3941). Another domain that is used in some embodiments is a histone phosphorylase or kinase, where examples include MSK1, MSK2, ATR, ATM, DNA-PK, Bubl, VprBP, IKK-a, PKCpi, Dik/Zip, JAK2, PKC5, WSTF and CK2. In some embodiments, a methylation domain is used and may be chosen from groups such as Ezh2, PRMT1/6, PRMT5/7, PRMT 2/6, CARM1, set7/9, MLL, ALL-1, Suv 39 h, G9a, SETDB1, Ezh2, Set2, Dotl, PRMT 1/6, PRMT 5/7, PR-Set7 and Suv4-20 h, Domains involved in sumoylation and biotinylation (Lys9, 13, 4, 18 and 12) may also be used in some embodiments (review see Kousarides (2007) Cell 128:693-705).

Fusion molecules are constructed by methods of cloning and biochemical conjugation that are well known to those of skill in the art. Fusion molecules comprise a DNA-binding domain and a functional domain (e.g., a transcriptional activation or repression domain). Fusion molecules also optionally comprise nuclear localization signals (such as, for example, that from the SV40 medium T-antigen) and epitope tags (such as, for example, FLAG and hemagglutinin). Fusion proteins (and nucleic acids encoding them) are designed such that the translational reading frame is preserved among the components of the fusion.

Fusions between a polypeptide component of a functional domain (or a functional fragment thereof) on the one hand, and a non-protein DNA-binding domain (e.g., antibiotic, intercalator, minor groove binder, nucleic acid) on the other, are constructed by methods of biochemical conjugation known to those of skill in the art. See, for example, the Pierce Chemical Company (Rockford, IL) Catalogue. Methods and compositions for making fusions between a minor groove binder and a polypeptide have been described. Mapp et al, (2000) Proc. Natl. Acad. Sci. USA 97:3930-3935. Likewise, CRISPR/Cas TFs and nucleases comprising a sgRNA nucleic acid component in association with a polypeptide component function domain are also known to those of skill in the art and detailed herein. b. Exogenous Polypeptide

In some embodiments, increased expression (i.e. overexpression) of the polynucleotide is mediated by introducing into the beta cell an exogenous polynucleotide encoding the polynucleotide to be overexpressed. In some embodiments, the exogenous polynucleotide is a recombinant nucleic acid. Well-known recombinant techniques can be used to generate recombinant nucleic acids as outlined herein.

In certain embodiments, the recombinant nucleic acids encoding an exogenous polynucleotide, such as a tolerogenic factor or a chimeric antigen receptor, may be operably linked to one or more regulatory nucleotide sequences in an expression construct. Regulatory nucleotide sequences will generally be appropriate for the host cell and recipient subject to be treated. Numerous types of appropriate expression vectors and suitable regulatory sequences are known in the art for a variety of host cells. Typically, the one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are also contemplated. The promoters may be either naturally occurring promoters, or hybrid promoters that combine elements of more than one promoter. An expression construct may be present in a cell on an episome, such as a plasmid, or the expression construct may be inserted in a chromosome. In a specific embodiment, the expression vector includes a selectable marker gene to allow the selection of transformed host cells. Certain embodiments include an expression vector comprising a nucleotide sequence encoding a variant polypeptide operably linked to at least one regulatory sequence. Regulatory sequence for use herein include promoters, enhancers, and other expression control elements. In certain embodiments, an expression vector is designed for the choice of the host cell to be transformed, the particular variant polypeptide desired to be expressed, the vector's copy number, the ability to control that copy number, and/or the expression of any other protein encoded by the vector, such as antibiotic markers.

In some embodiments, the exogenous polynucleotide is operably linked to a promoter for expression of the exogenous polynucleotide in the engineered islets. Examples of suitable mammalian promoters include, for example, promoters from the following genes: elongation factor 1 alpha (EF1a) promoter, ubiquitin/S27a promoter of the hamster (WO 97/15664), Simian vacuolating virus 40 (SV40) early promoter, adenovirus major late promoter, mouse metallothionein-I promoter, the long terminal repeat region of Rous Sarcoma Virus (RSV), mouse mammary tumor virus promoter (MMTV), Moloney murine leukemia virus Long Terminal repeat region, and the early promoter of human Cytomegalovirus (CMV). Examples of other heterologous mammalian promoters are the actin, immunoglobulin or heat shock promoter(s). In additional embodiments, promoters for use in mammalian host cells can be obtained from the genomes of viruses such as polyoma virus, fowlpox virus (UK 2,211,504 published 5 Jul. 1989), bovine papilloma virus, avian sarcoma virus, cytomegalovirus, a retrovirus, hepatitis-B virus and Simian Virus 40 (SV40). In further embodiments, heterologous mammalian promoters are used. Examples include the actin promoter, an immunoglobulin promoter, and heat-shock promoters. The early and late promoters of SV40 are conveniently obtained as an SV40 restriction fragment which also contains the SV40 viral origin of replication (Fiers et al, Nature 273: 113-120 (1978)). The immediate early promoter of the human cytomegalovirus is conveniently obtained as a HindIII restriction enzyme fragment (Greenaway et al, Gene 18: 355-360 (1982)). The foregoing references are incorporated by reference in their entirety.

In some embodiments, the expression vector is a bicistronic or multicistronic expression vector. Bicistronic or multicistronic expression vectors may include (1) multiple promoters fused to each of the open reading frames; (2) insertion of splicing signals between genes; (3) fusion of genes whose expressions are driven by a single promoter; and (4) insertion of proteolytic cleavage sites between genes (self-cleavage peptide) or insertion of internal ribosomal entry sites (IRESs) between genes.

In some embodiments, an expression vector or construct herein is a multicistronic construct. The terms “multicistronic construct” and “multicistronic vector” are used interchangeably herein and refer to a recombinant DNA construct that is to be transcribed into a single mRNA molecule, wherein the single mRNA molecule encodes two or more genes (e.g., two or more transgenes). The multi-cistronic construct is referred to as bicistronic construct if it encodes two genes, and tricistronic construct if it encodes three genes, and quadrocistronic construct if it encodes four genes, and so on.

In some embodiments, two or more exogenous polynucleotides comprised by a vector or construct (e.g., a transgene) are each separated by a multicistronic separation element. In some embodiments, the multicistronic separation element is an IRES or a sequence encoding a cleavable peptide or ribosomal skip element. In some embodiments, the multicistronic separation element is an IRES, such as an encephalomyocarditis (EMCV) virus IRES. In some embodiments, the multicistronic separation element is a cleavable peptide such as a 2A peptide. Exemplary 2A peptides include a P2A peptide, a T2A peptide, an E2A peptide, and an F2Apeptide. In some embodiments, the cleavable peptide is a T2A. In some embodiments, the two or more exogenous polynucleotides (e.g. the first exogenous polynucleotide and second exogenous polynucleotide) are operably linked to a promoter. In some embodiments, the first exogenous polynucleotide and the second exogenous polynucleotide are each operably linked to a promoter. In some embodiments, the promoter is the same promoter. In some embodiments, the promoter is an EF1 promoter.

In some cases, an exogenous polynucleotide encoding an exogenous polypeptide (e.g., an exogenous polynucleotide encoding a tolerogenic factor or complement inhibitor described herein) encodes a cleavable peptide or ribosomal skip element, such as T2A at the N-terminus or C-terminus of an exogenous polypeptide encoded by a multicistronic vector. In some embodiments, inclusion of the cleavable peptide or ribosomal skip element allows for expression of two or more polypeptides from a single translation initiation site. In some embodiments, the cleavable peptide is a T2A. In some embodiments, the T2A is or comprises the amino acid sequence set forth by SEQ ID NO: 11. In some embodiments, the T2A is or comprises the amino acid sequence set forth by SEQ ID NO: 12. In some embodiments, the T2A is or comprises the amino acid sequence set forth by SEQ ID NO: 17. In some embodiments, the T2A is or comprises the amino acid sequence set forth by SEQ ID NO: 18.

In some embodiments, the vector or construct includes a single promoter that drives the expression of one or more transcription units of an exogenous polynucleotide. In some embodiments, such vectors or constructs can be multicistronic (bicistronic or tricistronic, see e.g., U.S. Pat. No. 6,060,273). For example, in some embodiments, transcription units can be engineered as a bicistronic unit containing an IRES (internal ribosome entry site), which allows coexpression of gene products (e.g. one or more tolerogenic factors such as CD47) from an RNA transcribed from a single promoter. In some embodiments, the vectors or constructs provided herein are bicistronic, allowing the vector or construct to express two separate polypeptides. In some cases, the two separate polypeptides encoded by the vector or construct are tolerogenic factors (e.g., two factors selected from DUX4, B2M-HLA-E, CD35, CD52, CD16, CD52, CD47, CD46, CD55, CD59, CD27, CD200, HLA-C, HLA-E, HLA-E heavy chain, HLA-G, PD-L1, IDO1, CTLA4-Ig, Cl-Inhibitor, IL-10, IL-35, FASL, CCL21, MFGE8, SERPINB9, CD35, IL-39, CD16 Fc Receptor, IL15-RF, and H2-M3 (including any combination thereof). In some embodiments, the tolerogenic factor is two or more of CD47, PD-L1, HLA-E or HLA-G, CCL21, FasL, Serpinb9, CD200, and Mfge8 (including any combination thereof). In some embodiments, the two separate polypeptides encoded by the vector or construct are a tolerogenic factor (e.g., CD47). In some embodiments, the vectors or constructs provided herein are tricistronic, allowing the vector or construct to express three separate polypeptides. In some cases, the three nucleic acid sequences of the tricistronic vector or construct are a tolerogenic factor such as CD47. In some cases, the three nucleic acid sequences of the tricistronic vector or construct are three tolerogenic factors selected from DUX4, B2M-HLA-E, CD35, CD52, CD16, CD52, CD47, CD46, CD55, CD59, CD27, CD200, HLA-C, HLA-E, HLA-E heavy chain, HLA-G, PD-L1, IDO1, CTLA4-Ig, Cl-Inhibitor, IL-10, IL-35, FASL, CCL21, MFGE8, SERPINB9, CD35, IL-39, CD16 Fe Receptor, IL15-RF, and H2-M3 (including any combination thereof). In some embodiments, the three tolerogenic factor are selected from CD47, PD-L1, HLA-E or HLA-G, CCL21, FasL, Serpinb9, CD200, and Mfge8 (including any combination thereof). In some embodiments, the vectors or constructs provided herein are quadrocistronic, allowing the vector or construct to express four separate polypeptides. In some cases, the four separate polypeptides of the quadrocistronic vector or construct are four tolerogenic factors selected from DUX4, B2M-HLA-E, CD35, CD52, CD16, CD52, CD47, CD46, CD55, CD59, CD27, CD200, HLA-C, HLA-E, HLA-E heavy chain, HLA-G, PD-L1, IDO1, CTLA4-Ig, Cl-Inhibitor, IL-10, IL-35, FASL, CCL21, MFGE8, SERPINB9, CD35, IL-39, CD16 Fc Receptor, IL15-RF, and H2-M3 (including any combination thereof). In some embodiments, the four tolerogenic factor are selected from CD47, PD-L1, HLA-E or HLA-G, CCL21, FasL, Serpinb9, CD200, and Mfge8 (including any combination thereof). In some embodiments, the cell comprises one or more vectors or constructs, wherein each vector or construct is a monocistronic or a multicistronic construct as described above, and the monocistronic or multicistronic constructs encode one or more tolerogenic factors, in any combination or order.

In some embodiments, the cell comprises one or more vectors or constructs, wherein each vector or construct is a monocistronic or a multicistronic construct as described above, and the monocistronic or multicistronic constructs encode one or more tolerogenic factors, in any combination or order.

In some embodiments, a single promoter directs expression of an RNA that contains, in a single open reading frame (ORF), two, three, or four genes (e.g. encoding a tolerogenic factor (e.g., CD47)) separated from one another by sequences encoding a self-cleavage peptide (e.g., 2A sequences) or a protease recognition site (e.g., furin). The ORF thus encodes a single polypeptide, which, either during (in the case of 2A) or after translation, is processed into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known in the art. Examples of 2A sequences that can be used in the methods and nucleic acids disclosed herein include, without limitation, 2A sequences from the foot-and-mouth disease virus (F2A, e.g., SEQ ID NO: 16), equine rhinitis A virus (E2A, e.g., SEQ ID NO: 15), thosea asigna virus (T2A, e.g., SEQ ID NO: 11, 12, 17, or 18), and porcine teschovirus-1 (P2A, e.g., SEQ ID NO: 13 or 14) as described in U.S. Patent Publication No. 20070116690.

In cases where the vector or construct (e.g., transgene) contains more than one nucleic acid sequence encoding a protein, e.g., a first exogenous polynucleotide encoding CD47, and second exogenous polynucleotide encoding a second transgene, the vector or construct (e.g., transgene) may further include a nucleic acid sequence encoding a peptide between the first and second exogenous polynucleotide sequences. In some cases, the nucleic acid sequence positioned between the first and second exogenous polynucleotides encodes a peptide that separates the translation products of the first and second exogenous polynucleotides during or after translation. In some embodiments, the peptide contains a self-cleaving peptide or a peptide that causes ribosome skipping (a ribosomal skip element), such as a T2A, P2A, E2A or F2A peptide. In some embodiments, inclusion of the cleavable peptide or ribosomal skip element allows for expression of two or more polypeptides from a single translation initiation site. In some embodiments, the peptide is a self-cleaving peptide that is a T2A peptide. In some embodiments, the T2A is or comprises the amino acid sequence set forth by SEQ ID NO: 11. In some embodiments, the T2A is or comprises the amino acid sequence set forth by SEQ ID NO: 12. In some embodiments, the T2A is or comprises the amino acid sequence set forth by SEQ ID NO: 17. In some embodiments, the T2A is or comprises the amino acid sequence set forth by SEQ ID NO: 18. In some embodiments, the P2A is or comprises the amino acid sequence set forth by SEQ ID NO: 13. In some embodiments, the P2A is or comprises the amino acid sequence set forth by SEQ ID NO: 14. In some embodiments, the E2A is or comprises the amino acid sequence set forth by SEQ ID NO: 15. In some embodiments, the F2A is or comprises the amino acid sequence set forth by SEQ ID NO: 16.

The process of introducing the polynucleotides described herein into beta cells can be achieved by any suitable technique. Suitable techniques include calcium phosphate or lipid-mediated transfection, electroporation, transposase-mediated delivery, and transduction or infection using a viral vector. In some embodiments, the polynucleotides are introduced into a cell via viral transduction (e.g., lentiviral transduction) or otherwise delivered on a viral vector (e.g., fusogen-mediated delivery). In some embodiments, vectors that package a polynucleotide encoding an exogenous polynucleotide may be used to deliver the packaged polynucleotides to a cell or population of cells. These vectors may be of any kind, including DNA vectors, RNA vectors, plasmids, viral vectors and particles. In some embodiments, lipid nanoparticles can be used to deliver an exogenous polynucleotide to a cell. In some embodiments, viral vectors can be used to deliver an exogenous polynucleotide to a cell. Viral vector technology is well known and described in Sambrook et al. (2001, Molecular Cloning: A Laboratory Manual, Cold Spring Harbor Laboratory, New York). Viruses, which are useful as vectors include, but are not limited to lentiviral vectors, adenoviral vectors, adeno-associated viral (AAV) vectors, herpes simplex viral vectors, retroviral vectors, oncolytic viruses, and the like. In some embodiments, the introduction of the exogenous polynucleotide into the cell can be specific (targeted) or non-specific (e.g. non-targeted). In some embodiments, the introduction of the exogenous polynucleotide into the cell can result in integration or insertion into the genome in the cell. In other embodiments, the introduced exogenous polynucleotide may be non-integrating or episomal in the cell. A skilled artisan is familiar with methods of introducing nucleic acid transgenes into a cell, including any of the exemplary methods described herein, and can choose a suitable method.

### 1) Non-Targeted Delivery

In some embodiments, an exogenous polynucleotide is introduced into a beta cell (e.g. source cell) by any of a variety of non-targeted methods. In some embodiments, the exogenous polynucleotide is inserted into a random genomic locus of a host cell. As known to a person skilled in the art, viral vectors, including, for example, retroviral vectors and lentiviral vectors are commonly used to deliver genetic material into host cells and randomly insert the foreign or exogenous gene into the host cell genome to facilitate stable expression and replication of the gene. In some embodiments, the non-targeted introduction of the exogenous polynucleotide into the cell is under conditions for stable expression of the exogenous polynucleotide in the cell. In some embodiments, methods for introducing a nucleic acid for stable expression in a cell involves any method that results in stable integration of the nucleic acid into the genome of the cell, such that it may be propagated if the cell it has integrated into divides.

In some embodiments, the viral vector is a lentiviral vector. Lentiviral vectors are particularly useful means for successful viral transduction as they permit stable expression of the gene contained within the delivered nucleic acid transcript. Lentiviral vectors express reverse transcriptase and integrase, two enzymes required for stable expression of the gene contained within the delivered nucleic acid transcript. Reverse transcriptase converts an RNA transcript into DNA, while integrase inserts and integrates the DNA into the genome of the target cell. Once the DNA has been integrated stably into the genome, it divides along with the host. The gene of interest contained within the integrated DNA may be expressed constitutively or it may be inducible. As part of the host cell genome, it may be subject to cellular regulation, including activation or repression, depending on a host of factors in the target cell.

Lentiviruses are subgroup of the Retroviridae family of viruses, named because reverse transcription of viral RNA genomes to DNA is required before integration into the host genome. As such, the most important features of lentiviral vehicles/particles are the integration of their genetic material into the genome of a target/host cell. Some examples of lentivirus include the Human Immunodeficiency Viruses: HIV-1 and HIV-2, the Simian Immunodeficiency Virus (SIV), feline immunodeficiency virus (FIV), bovine immunodeficiency virus (BIV), Jembrana Disease Virus (JDV), equine infectious anemia virus (EIAV), equine infectious anemia, virus, visna-maedi and caprine arthritis encephalitis virus (CAEV).

Typically, lentiviral particles making up the gene delivery vehicle are replication defective on their own (also referred to as “self-inactivating”). Lentiviruses are able to infect both dividing and non-dividing cells by virtue of the entry mechanism through the intact host nuclear envelope (Naldini L et al., Curr. Opin. Bioiecknol, 1998, 9: 457-463). Recombinant lentiviral vehicles/particles have been generated by multiply attenuating the HIV virulence genes, for example, the genes Env, Vif, Vpr, Vpu, Nef and Tat are deleted making the vector biologically safe. Correspondingly, lentiviral vehicles, for example, derived from HIV-1/HIV-2 can mediate the efficient delivery, integration and long-term expression of transgenes into non-dividing cells.

Lentiviral particles may be generated by co-expressing the virus packaging elements and the vector genome itself in a producer cell such as human HEK293T cells. These elements are usually provided in three (in second generation lentiviral systems) or four separate plasmids (in third generation lentiviral systems). The producer cells are co-transfected with plasmids that encode lentiviral components including the core (i.e. structural proteins) and enzymatic components of the virus, and the envelope protein(s) (referred to as the packaging systems), and a plasmid that encodes the genome including a foreign transgene, to be transferred to the target cell, the vehicle itself (also referred to as the transfer vector). In general, the plasmids or vectors are included in a producer cell line. The plasmids/vectors are introduced via transfection, transduction or infection into the producer cell line. Methods for transfection, transduction or infection are well known by those of skill in the art. As non-limiting example, the packaging and transfer constructs can be introduced into producer cell lines by calcium phosphate transfection, lipofection or electroporation, generally together with a dominant selectable marker, such as neomycin (neo), dihydrofolate reductase (DHFR), glutamine synthetase or adenosine deaminase (ADA), followed by selection in the presence of the appropriate drug and isolation of clones.

The producer cell produces recombinant viral particles that contain the foreign gene, for example, the polynucleotides encoding the exogenous polynucleotide. The recombinant viral particles are recovered from the culture media and titrated by standard methods used by those of skill in the art. The recombinant lentiviral vehicles can be used to infect target cells, such source cells including any described in Section IIC.

Cells that can be used to produce high-titer lentiviral particles may include, but are not limited to, HEK293T cells, 293G cells, STAR cells (Relander et al., Mol Ther. 2005, 11: 452-459), FreeStyle™ 293 Expression System (ThermoFisher, Waltham, MA), and other HEK293T- based producer cell lines (e.g., Stewart et al., Hum Gene Ther. 2011, 2,2.(3):357˜369; Lee et al, Biotechnol Bioeng, 2012, 10996): 1551-1560; Throm et al., Blood. 2009, 113(21): 5104-5110).

Additional elements provided in lentiviral particles may comprise retroviral LTR (long-terminal repeat) at either 5′ or 3′ terminus, a retroviral export element, optionally a lentiviral reverse response element (RRE), a promoter or active portion thereof, and a locus control region (LCR) or active portion thereof. Other elements include central polypurine tract (cPPT) sequence to improve transduction efficiency in non-dividing cells, Woodchuck Hepatitis Virus (WHP) Posttranscriptional Regulatory Element (WPRE) which enhances the expression of the transgene, and increases titer.

Methods for generating recombinant lentiviral particles are known to a skilled artisan, for example, U.S. Pat. Nos. 8,846,385; 7,745,179; 7,629,153; 7,575,924; 7,179,903; and 6,808,905. Lentivirus vectors used may be selected from, but are not limited to pLVX, pLenti, pLenti6, pLJMI, FUGW, pWPXL, pWPI, pLenti CMV puro DEST, pLJMI-EGFP, pULTRA, pInducer2Q, pHIV-EGFP, pCW57.1, pTRPE, pELPS, pRRL, and pLionII, Any known lentiviral vehicles may also be used (See, U.S. Pat. Nos. 9,260,725: 9,068,199: 9,023,646: 8,900,858: 8,748,169; 8,709,799; 8,420,104; 8,329,462; 8,076,106; 6,013,516; and 5,994, 136; International Patent Publication NO.: WO2012079000).

In some embodiments, the exogenous polynucleotide is introduced into the cell under conditions for transient expression of the cell, such as by methods that result in episomal delivery of an exogenous polynucleotide.

In some embodiments, polynucleotides encoding the exogenous polynucleotide may be packaged into recombinant adeno-associated viral (rAAV) vectors. Such vectors or viral particles may be designed to utilize any of the known serotype capsids or combinations of serotype capsids. The serotype capsids may include capsids from any identified AAV serotypes and variants thereof, for example, AAV1, AAV2, AAV2G9, AAV3, AAV4, AAV4-4, AAV5, AAV6, AAV7, AAV8, AAV9, AAV10, AAV 11, AAV12 and AAVrh10. In some embodiments, the AAV serotype may be or have a sequence as described in United States Publication No. US20030138772; Pulicherla et al. Molecular Therapy, 2011, 19(6): 1070-1078; U.S. Pat. Nos. 6,156,303; 7,198,951; U.S. Patent Publication Nos. US2015/0159173 and US2014/0359799; and International Patent Publication nos.: WO1998/011244, WO2005/033321 and WO2014/14422.

AAV vectors include not only single stranded vectors but self-complementary AAV vectors (scAAVs). scAAV vectors contain DNA which anneals together to form double stranded vector genome. By skipping second strand synthesis, scAAVs allow for rapid expression in the cell. The rAAV vectors may be manufactured by standard methods in the art such as by triple transfection, in sf9 insect cells or in suspension cell cultures of human cells such as HEK293 cells.

In some embodiments, non-viral based methods may be used. For instance, in some aspects, vectors comprising the polynucleotides may be transferred to cells by non-viral methods by physical methods such as needles, electroporation, sonoporation, hyrdoporation; chemical carriers such as inorganic particles (e.g. calcium phosphate, silica, gold) and/or chemical methods. In other aspects, synthetic or natural biodegradable agents may be used for delivery such as cationic lipids, lipid nano emulsions, nanoparticles, peptide-based vectors, or polymer-based vectors.

### 2) Targeted Delivery

The exogenous polynucleotide can be inserted into any suitable target genomic loci of the beta cell. In some embodiments, the exogenous polynucleotide is introduced into the cell by targeted integration into a target loci. In some embodiments, targeted integration can be achieved by gene editing using one or more nucleases and/or nickases and a donor template in a process involving homology-dependent or homology-independent recombination.

A number of gene editing methods can be used to insert an exogenous polynucleotide into the specific genomic locus of choice, including for example homology-directed repair (HOR), homology-mediated end-joining (HMEJ), homology-independent targeted integration (HITI), obligate ligation-gated recombination (ObliGaRe), or precise integration into target chromosome (PITCh).

In some embodiments, the nucleases create specific double-strand breaks (DSBs) at desired locations (e.g. target sites) in the genome, and harness the cell's endogenous mechanisms to repair the induced break. The nickases create specific single-strand breaks at desired locations in the genome. In one non-limiting example, two nickases can be used to create two single-strand breaks on opposite strands of a target DNA, thereby generating a blunt or a sticky end. Any suitable nuclease can be introduced into a cell to induce genome editing of a target DNA sequence including, but not limited to, CRISPR-associated protein (Cas) nucleases, zinc finger nucleases (ZFNs), transcription activator-like effector nucleases (TALENs), meganucleases, other endo- or exo-nucleases, variants thereof, fragments thereof, and combinations thereof. In some embodiments, when a nuclease or a nickase is introduced with a donor template containing an exogenous polynucleotide sequence (also called a transgene) flanked by homology sequences (e.g. homology arms) that are homologous to sequences at or near the endogenous genomic target locus, e.g. a safe harbor locus, DNA damage repair pathways can result in integration of the transgene sequence at the target site in the cell. This can occur by a homology-dependent process. In some embodiments, the donor template is a circular double-stranded plasmid DNA, single-stranded donor oligonucleotide (ssODN), linear double-stranded polymerase chain reaction (PCR) fragments, or the homologous sequences of the intact sister chromatid. Depending on the form of the donor template, the homology-mediated gene insertion and replacement can be carried out via specific DNA repair pathways such as homology-directed repair (HDR), synthesis-dependent strand annealing (SDSA), microhomology-mediated end joining (MMEJ), and homology-mediated end joining (HMEJ) pathways.

For instance, DNA repair mechanisms can be induced by a nuclease after (i) two SSBs, where there is a SSB on each strand, thereby inducing single strand overhangs; or (ii) a DSB occurring at the same cleavage site on both strands, thereby inducing a blunt end break. Upon cleavage by one of these agents, the target locus with the SSBs or the DSB undergoes one oftwo major pathways for DNA damage repair: (1) the error-prone non-homologous end joining (NHEJ), or (2) the high-fidelity homology-directed repair (HDR) pathway. In some embodiments, a donor template (e.g. circular plasmid DNA or a linear DNA fragment, such as a ssODN) introduced into cells in which there are SSBs or a DSB can result in HDR and integration of the donor template into the target locus. In general, in the absence of a donor template, the NHEJ process re-ligates the ends of the cleaved DNA strands, which frequently results in nucleotide deletions and insertions at the cleavage site.

In some embodiments, site-directed insertion of the exogenous polynucleotide into a cell may be achieved through HDR-based approaches. HDR is a mechanism for cells to repair double-strand breaks (DSBs) in DNA and can be utilized to modify genomes in many organisms using various gene editing systems, including clustered regularly interspaced short palindromic repeat (CRISPR)/Cas systems, zinc finger nucleases (ZFNs), transcription activator-like effector nucleases (TALENs), meganucleases, and transposases.

In some embodiments, the targeted integration is carried by introducing one or more sequence-specific or targeted nucleases, including DNA-binding targeted nucleases and gene editing nucleases such as zinc finger nucleases (ZFN) and transcription activator-like effector nucleases (TALENs), and RNA-guided nucleases such as a CRISPR-associated nuclease (Cas) system, specifically designed to be targeted to at least one target site(s) sequence of a target gene. Exemplary ZFNs, TALEs, and TALENs are described in, e.g., Lloyd et al., Frontiers in Immunology, 4(221): 1-7 (2013). In particular embodiments, targeted genetic disruption at or near the target site is carried out using clustered regularly interspaced short palindromic repeats (CRISPR) and CRISPR-associated (Cas) proteins. See Sander and Joung, (2014) Nature Biotechnology, 32(4): 347-355.

Any of the systems for gene disruption described in Section II. A.1 can be used and, when also introduced with an appropriate donor template having with an exogenous polynucleotide, e.g. transgene sequences, can result in targeted integration of the exogenous polynucleotide at or near the target site of the genetic disruption. In particular embodiments, the genetic disruption is mediated using a CRISPR/Cas system containing one or more guide RNAs (gRNA) and a Cas protein. Exemplary Cas proteins and gRNA are described in Section II.A above, any of which can be used in HDR mediated integration of an exogenous polynucleotide into a target locus to which the Crispr/Cas system is specific for. It is within the level of a skilled artisan to choose an appropriate Cas nuclease and gRNA, such as depending on the particular target locus and target site for cleavage and integration of the exogenous polynucleotide by HDR. Further, depending on the target locus a skilled artisan can readily prepare an appropriate donor template, such as described further below.

In some embodiments, the DNA editing system is an RNA-guided CRISPR/Cas system (such as RNA-based CRISPR/Cas system), wherein the CRISPR/Cas system is capable of creating a double-strand break in the target locus (e.g. safe harbor locus) to induce insertion of the transgene into the target locus. In some embodiments, the nuclease system is a CRISPR/Cas9 system. In some embodiments, the CRISPR/Cas9 system comprises a plasmid-based Cas9. In some embodiments, the CRISPR/Cas9 system comprises a RNA-based Cas9. In some embodiments, the CRISPR/Cas9 system comprises a Cas9 mRNA and gRNA. In some embodiments, the CRISPR/Cas9 system comprises a protein/RNA complex, or a plasmid/RNA complex, or a protein/plasmid complex. In some embodiments, there are provided methods for generating modified cells, which comprises introducing into a source cell (e.g. a beta cell) a donor template containing a transgene or exogenous polynucleotide sequence and a DNA nuclease system including a DNA nuclease system (e.g. Cas9) and a locus-specific gRNA. In some embodiments, the Cas9 is introduced as an mRNA. In some embodiments, the Cas9 is introduced as a ribonucleoprotein complex with the gRNA.

Generally, the donor template to be inserted would comprise at least the transgene cassette containing the exogenous polynucleotide of interest (e.g., the tolerogenic factor or CAR) and would optionally also include the promoter. In certain of these embodiments, the transgene cassette containing the exogenous polynucleotide and/or promoter to be inserted would be flanked in the donor template by homology arms with sequences homologous to sequences immediately upstream and downstream of the target cleavage site, i.e., left homology arm (LHA) and right homology arm (RHA). Typically, the homology arms of the donor template are specifically designed for the target genomic locus to serve as template for HDR. The length of each homology arm is generally dependent on the size of the insert being introduced, with larger insertions requiring longer homology arms.

In some embodiments, a donor template (e.g., a recombinant donor repair template) comprises: (i) a transgene cassette comprising an exogenous polynucleotide sequence (for example, a transgene operably linked to a promoter, for example, a heterologous promoter); and (ii) two homology arms that flank the transgene cassette and are homologous to portions of a target locus (e.g. safe harbor locus) at either side of a DNA nuclease (e.g., Cas nuclease, such as Cas9 or Cas12) cleavage site. The donor template can further comprise a selectable marker, a detectable marker, and/or a purification marker.

In some embodiments, the homology arms are the same length. In other embodiments, the homology arms are different lengths. The homology arms can be at least about 10 base pairs (bp), e.g., at least about 10 bp, 15 bp, 20 bp, 25 bp, 30 bp, 35 bp, 45 bp, 55 bp, 65 bp, 75 bp, 85 bp, 95 bp, 100 bp, 150 bp, 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 950 bp, 1000 bp, 1.1 kilobases (kb), 1.2 kb, 1.3 kb, 1.4 kb, 1.5 kb, 1.6 kb, 1.7 kb, 1.8 kb, 1.9 kb, 2.0 kb, 2.1 kb, 2.2 kb, 2.3 kb, 2.4 kb, 2.5 kb, 2.6 kb, 2.7 kb, 2.8 kb, 2.9 kb, 3.0 kb, 3.1 kb, 3.2 kb, 3.3 kb, 3.4 kb, 3.5 kb, 3.6 kb, 3.7 kb, 3.8 kb, 3.9 kb, 4.0 kb, or longer. The homology arms can be about 10 bp to about 4 kb, e.g., about 10 bp to about 20 bp, about 10 bp to about 50 bp, about 10 bp to about 100 bp, about 10 bp to about 200 bp, about 10 bp to about 500 bp, about 10 bp to about 1 kb, about 10 bp to about 2 kb, about 10 bp to about 4 kb, about 100 bp to about 200 bp, about 100 bp to about 500 bp, about 100 bp to about 1 kb, about 100 bp to about 2 kb, about 100 bp to about 4 kb, about 500 bp to about 1 kb, about 500 bp to about 2 kb, about 500 bp to about 4 kb, about 1 kb to about 2 kb, about 1 kb to about 2 kb, about 1 kb to about 4 kb, or about 2 kb to about 4 kb.

In some embodiments, the donor template can be cloned into an expression vector. Conventional viral and non-viral based expression vectors known to those of ordinary skill in the art can be used.

In some embodiments, the target locus targeted for integration may be any locus in which it would be acceptable or desired to target integration of an exogenous polynucleotide or transgene. Non-limiting examples of a target locus include, but are not limited to, a CXCR4 gene, an albumin gene, a SHS231 locus, an F3 gene (also known as CD142), a MICA gene, a MICB gene, a LRP1 gene (also known as CD91), a HMGB1 gene, an ABO gene, a RHD gene, a FUT1 gene, a KDM5D gene (also known as HY), a B2M gene, a CIITA gene, a CCR5 gene, a F3 (i.e., CD142) gene, a LRP1 gene, a HMGB1 gene, an ABO gene, a RHD gene, a FUT1 gene, a KDM5D (i.e., HY) gene, a PDGFRa gene, a OLIG2 gene, and/or a GFAP gene. In some embodiments, the exogenous polynucleotide can be inserted in a suitable region of the target locus (e.g. safe harbor locus), including, for example, an intron, an exon, and/or gene coding region (also known as a Coding Sequence, or “CDS”). In some embodiments, the insertion occurs in one allele of the target genomic locus. In some embodiments, the insertion occurs in both alleles of the target genomic locus. In either of these embodiments, the orientation of the transgene inserted into the target genomic locus can be either the same or the reverse of the direction of the gene in that locus.

In some embodiments, the exogenous polynucleotide is interested into an intron, exon, or coding sequence region of the safe harbor gene locus. In some embodiments, the exogenous polynucleotide is inserted into an endogenous gene wherein the insertion causes silencing or reduced expression of the endogenous gene. Exemplary genomic loci for insertion of an exogenous polynucleotide are depicted in Table 4.

TABLE 4
Exemplary genomic loci for insertion of exogenous polynucleotides

| | | | | Target region | |
|---|---|---|---|---|---|
| Number | species | name | Ensembl ID | for cleavage | Also known as |
| 1 | human | B2M | ENSG00000166710 | CDS | |
| 2 | human | CIITA | ENSG00000179583 | CDS | |
| 3 | human | TRAC | ENSG00000277734 | CDS | |
| 4 | human | PPP1R12C | ENSG00000125503 | Intron 1 and 2 | AAVS1 |
| 5 | human | CLYBL | ENSG00000125246 | Intron 2 | |
| 6 | human | CCR5 | ENSG00000160791 | Exons 1-3, | |
| | | | | introns 1-2, and | |
| | | | | CDS | |
| 7 | human | THUMPD3-AS1 | ENSG00000206573 | Intron 1 | ROSA26 |
| 8 | human | Ch-4: 58,976,613 | | 500 bp window | SHS231 |
| 9 | human | F3 | ENSG00000117525 | CDS | CD142 |
| 10 | human | MICA | ENSG00000204520 | CDS | |
| 11 | human | MICB | ENSG00000204516 | CDS | |
| 12 | human | LRP1 | ENSG00000123384 | CDS | |
| 13 | human | HMGB1 | ENSG00000189403 | CDS | |
| 14 | human | ABO | ENSG00000175164 | CDS | |
| 15 | human | RHD | ENSG00000187010 | CDS | |
| 16 | human | FUT1 | ENSG00000174951 | CDS | |
| 17 | human | KDM5D | ENSG00000012817 | CDS | HY |

In some embodiments, the target locus is a safe harbor locus. In some embodiments, a safe harbor locus is a genomic location that allows for stable expression of integrated DNA with minimal impact on nearby or adjacent endogenous genes, regulatory element and the like. In some cases, a safe harbor gene enables sustainable gene expression and can be targeted by engineered nuclease for gene modification in various cell types including beta cells, including derivatives thereof, and differentiated cells thereof. Non-limiting examples of a safe harbor locus include, but are not limited to, a CCR5 gene locus, a PPP1R12C (also known as AAVS1) gene locus, a CLYBL gene locus, and/or a Rosa gene locus (e.g., ROSA26 gene locus). n some embodiments, the safe harbor locus is selected from the group consisting of the AAVS1 locus, the CCR5 locus, and the CLYBL locus. In some cases SHS231 can be targeted as a safe harbor locus in many cell types. In some cases, certain loci can function as a safe harbor locus in certain cell types. For instance, PDGFRa is a safe harbor for glial progenitor cells (GPCs), OLIG2 is a safe harbor locus for oligodendrocytes, and GFAP is a safe harbor locus for astrocytes. It is within the level of a skilled artisan to choose an appropriate safe harbor locus for the engineered islets. In some cases, more than one safe harbor gene can be targeted, thereby introducing more than one transgene into the genetically modified cell.

In some embodiments, there are provided methods for generating engineered islets, which comprises introducing into a source cell (e.g. a primary islet cell) a donor template containing a transgene or exogenous polynucleotide sequence and a DNA nuclease system including a DNA nuclease system (e.g. Cas9) and a locus-specific gRNA that comprise complementary portions (e.g. gRNA targeting sequence) specific to a CCR5 gene locus, a PPP1R12C (also known as AAVS1) gene locus, a CLYBL gene locus, and/or a Rosa gene locus (e.g., ROSA26 gene locus). In some embodiments, the genomic locus targeted by the gRNAs is located within 4000 bp, within 3500 bp, within 3000 bp, within 2500 bp, within 2000 bp, within 1500 bp, within 1000 bp, or within 500 bp of any of the loci as described.

In some embodiments, the gRNAs used herein for HDR-mediated insertion of a transgene comprise a complementary portion (e.g. gRNA targeting sequence) that recognizes a target sequence in AAVS1. In certain of these embodiments, the target sequence is located in intron 1 of AAVS 1. AAVS1 is located at Chromosome 19: 55,090,918-55,117,637 reverse strand, and AAVS1 intron 1 (based on transcript ENSG00000125503) is located at Chromosome 19: 55,117,222-55,112,796 reverse strand. In certain embodiments, the gRNAs target a genomic locus within 4000 bp, within 3500 bp, within 3000 bp, within 2500 bp, within 2000 bp, within 1500 bp, within 1000 bp, or within 500 bp of Chromosome 19: 55, 117,222-55, 112,796. In certain embodiments, the gRNAs target a genomic locus within 4000 bp, within 3500 bp, within 3000 bp, within 2500 bp, within 2000 bp, within 1500 bp, within 1000 bp, or within 500 bp of Chromosome 19: 55,115,674. In certain embodiments, the gRNA is configured to produce a cut site at Chromosome 19: 55, 115,674, or at a position within 5, 10, 15, 20, 30, 40 or 50 nucleotides of Chromosome 19: 55, 115,674. In certain embodiments, the gRNA is GET000046, also known as “sgAAVS1-1,” described in Li et al., Nat. Methods 16:866-869 (2019). This gRNA comprises a complementary portion (e.g. gRNA targeting sequence) having the nucleic acid sequence set forth in SEQ ID NO: 22 (e.g. Table 5) and targets intron 1 of AAVS1 (also known as PPP1R12C).

In some embodiments, the gRNAs used herein for HDR-mediated insertion of a transgene comprise a complementary portion (e.g. gRNA targeting sequence) that recognizes a target sequence in CLYBL. In certain of these embodiments, the target sequence is located in intron 2 of CL YBL. CLYBL is located at Chromosome 13: 99,606,669-99,897, 134 forward strand, and CLYBL intron 2 (based on transcript ENST00000376355.7) is located at Chromosome 13: 99,773,011-99,858,860 forward strand. In certain embodiments, the gRNAs target a genomic locus within 4000 bp, within 3500 bp, within 3000 bp, within 2500 bp, within 2000 bp, within 1500 bp, within 1000 bp, or within 500 bp of Chromosome 13: 99,773,011-99,858,860. In certain embodiments, the gRNAs target a genomic locus within 4000 bp, within 3500 bp, within 3000 bp, within 2500 bp, within 2000 bp, within 1500 bp, within 1000 bp, or within 500 bp of Chromosome 13: 99,822,980. In certain embodiments, the gRNA is configured to produce a cut site at Chromosome 13: 99,822,980, or at a position within 5, 0, 15, 20, 30, 40 or 50 nucleotides of Chromosome 13: 99,822,980. In certain embodiments, the gRNA is GET000047, which comprises a complementary portion (e.g. gRNA targeting sequence) having the nucleic acid sequence set forth in SEQ ID NO: 23 (e.g. Table 5) and targets intron 2 of CLYBL. The target site is similar to the target site of the TALENs as described in Cerbini et al., pLoS One, 10(1): e0116032 (2015).

In some embodiments, the gRNAs used herein for HDR-mediated insertion of a transgene comprise a complementary portion (e.g. gRNA targeting sequence) that recognizes a target sequence in CCR5. In certain of these embodiments, the target sequence is located in exon 3 of CCR5. CCR5 is located at Chromosome 3: 46,370,854-46,376,206 forward strand, and CCR5 exon 3 (based on transcript ENST00000292303.4) is located at Chromosome 3: 46,372,892-46,376,206 forward strand. In certain embodiments, the gRNAs target a genomic locus within 4000 bp, within 3500 bp, within 3000 bp, within 2500 bp, within 2000 bp, within 1500 bp, within 1000 bp, or within 500 bp of Chromosome 3: 46,372,892-46,376,206. In certain embodiments, the gRNAs target a genomic locus within 4000 bp, within 3500 bp, within 3000 bp, within 2500 bp, within 2000 bp, within 1500 bp, within 1000 bp, or within 500 bp of Chromosome 3: 46,373,180. In certain embodiments, the gRNA is configured to produce a cut site at Chromosome 3: 46,373,180, or at a position within 5, 10, 15, 20, 30, 40, or 50 nucleotides of Chromosome 3: 46,373,180. In certain embodiments the gRNA is GET000048, also known as “crCCR5_D,” described in Mandal et al., Cell Stem Cell 15:643-652 (2014). This gRNA comprises a complementary portion having the nucleic acid sequence set forth in SEQ ID NO: 24 (e.g. Table 5) and targets exon 3 of CCR5 (alternatively annotated as exon 2 in the Ensembl genome database). See Gomez-Ospina et al., Nat. Comm. 10(1):4045 (2019).

Table 5 sets forth exemplary gRNA targeting sequences. As is understood by a skilled artisan, the gRNA targeting sequence will comprise the base uracil (U), whereas DNA encoding the gRNA targeting sequence will comprise the base thymine (T) as shown in the DNA complementary portion sequences set forth in Table 5. It will be understood by one of ordinary skill in the art that uracil and thymine can both be represented by ‘t’, instead of ‘u’ for uracil and ‘t’ for thymine; in the context of a ribonucleic acid, it will be understood that ‘t’ is used to represent uracil unless otherwise indicated. While not wishing to be bound by theory, in some embodiments, it is believed that the complementarity of the guide sequence with the target sequence contributes to specificity of the interaction of the gRNA molecule/Cas molecule complex with a target nucleic acid. It is understood that in a guide sequence and target sequence pair, the uracil bases in the guide sequence will pair with the adenine bases in the target sequence.

TABLE 5
Exemplary gRNA targeting sequences for CCR5

| | Nucleic Acid | SEQ ID |
|---|---|---|
| Description | Sequence | NO |
| GET000046 guide | (5′→3′)accccacag | 22 |
| | tggggccacta | |
| GET000047 guide | (5′→3′)tgttggaag | 23 |
| | gatgaggaaat | |
| G ET000048 guide | (5′→3′)tcactatgc | 24 |
| | tgccgcccagt | |

In some embodiments, the target locus is a locus that is desired to be knocked out in the cells. In such embodiments, such a target locus is any target locus whose disruption or elimination is desired in the cell, such as to modulate a phenotype or function of the cell. For instance, any of the gene modifications described herein to reduce expression of a target gene may be a desired target locus for targeted integration of an exogenous polynucleotide, in which the genetic disruption or knockout of a target gene and overexpression by targeted insertion of an exogenous polynucleotide may be achieved at the same target site or locus in the cell. For instance, the HDR process may be used to result in a genetic disruption to eliminate or reduce expression of (e.g. knock out) any target gene set forth in Table 1b while also integrating (e.g. knocking in) an exogenous polynucleotide into the target gene by using a donor template with flanking homology arms that are homologous to nucleic acid sequences at or near the target site of the genetic disruption.

In some embodiments, there are provided methods for generating engineered islets, which comprises introducing into a source cell (e.g. a beta cell) a donor template containing a transgene or exogenous polynucleotide sequence and a DNA nuclease system including a DNA nuclease system (e.g. Cas9) and a locus-specific gRNA that comprise complementary portions specific to the B2M locus or the CIITA locus. In some embodiments, the genomic locus targeted by the gRNAs is located within 4000 bp, within 3500 bp, within 3000 bp, within 2500 bp, within 2000 bp, within 1500 bp, within 1000 bp, or within 500 bp of any of the loci as described.

In particular embodiments, the target locus is B2M. In some embodiments, the engineered islets comprise a genetic modification targeting the B2M gene. In some embodiments, the genetic modification targeting the B2M gene is by using a targeted nuclease system that comprises a Cas protein or a polynucleotide encoding a Cas protein, and at least one guide ribonucleic acid sequence for specifically targeting the B2M gene. In some embodiments, the at least one guide ribonucleic acid (gRNA) sequence for specifically targeting the B2M gene is selected from the group consisting of SEQ ID NOS: 81240-85644 of Appendix 2 or Table 15 of WO2016/183041, the disclosure is incorporated by reference in its entirety. In some embodiments, an exogenous polynucleotide is integrated into the disrupted B2M locus by HDR by introducing a donor template containing the exogenous polynucleotide sequence with flanking homology arms homologous to sequences adjacent to the target site targeted by the gRNA.

In particular embodiments, the target locus is CIITA. In some embodiments, the engineered islets comprise a genetic modification targeting the CIITA gene. In some embodiments, the genetic modification targeting the CIITA gene is by a targeted nuclease system that comprises a Cas protein or a polynucleotide encoding a Cas protein, and at least one guide ribonucleic acid sequence for specifically targeting the CIITA gene. In some embodiments, the at least one guide ribonucleic acid sequence for specifically targeting the CIITA gene is selected from the group consisting of SEQ ID NOS: 5184-36352 of Appendix 1 or Table 12 of WO2016183041, the disclosure is incorporated by reference in its entirety. In some embodiments, an exogenous polynucleotide is integrated into the disrupted CIITA locus by HDR by introducing a donor template containing the exogenous polynucleotide sequence with flanking homology arms homologous to sequences adjacent to the target site targeted by the gRNA.

In some embodiments, it is within the level of a skilled artisan to identify new loci and/or gRNA sequences for use in HDR-mediated integration approaches as described. For example, for CRISPR/Cas systems, when an existing gRNA for a particular locus (e.g., within a target gene, e.g. set forth in Table 1b) is known, an “inch worming” approach can be used to identify additional loci for targeted insertion of transgenes by scanning the flanking regions on either side of the locus for PAM sequences, which usually occurs about every 100 base pairs (bp) across the genome. The PAM sequence will depend on the particular Cas nuclease used because different nucleases usually have different corresponding PAM sequences. The flanking regions on either side of the locus can be between about 500 to 4000 bp long, for example, about 500 bp, about 1000 bp, about 1500 bp, about 2000 bp, about 2500 bp, about 3000 bp, about 3500 bp, or about 4000 bp long. When a PAM sequence is identified within the search range, a new guide can be designed according to the sequence of that locus for use in genetic disruption methods. Although the CRISPR/Cas system is described as illustrative, any HDR-mediated approaches as described can be used in this method of identifying new loci, including those using ZFNs, TALENS, meganucleases and transposases.

In some embodiments, the exogenous polynucleotide encodes an exogenous CD47 polypeptide (e.g., a human CD47 polypeptide) and the exogenous polypeptide is inserted into a safe harbor gene loci or a safe harbor site as disclosed herein or a genomic locus that causes silencing or reduced expression of the endogenous gene. In some embodiments, the exogenous polynucleotide encoding CD47 is inserted in a CCR5 gene locus, a PPP1R12C (also known as AAVS1) gene locus, a CLYBL gene locus, and/or a Rosa gene locus (e.g., ROSA26 gene locus). In some embodiments, the polynucleotide is inserted in a B2M, CIITA, PD1 or CTLA4 gene locus.

C. Exemplary Embodiments of Engineered islets

In some embodiments, the engineered islets exhibit increased expression of CD47 and reduced expression of one or more molecules of the MHC class I complex. In some embodiments, the engineered islets exhibit increased expression of CD47 and reduced expression of one or more molecules of the MHC class I and MHC class II complexes. In some embodiments, the engineered islets express one or more exogenous complement inhibitor polypeptides selected from CD46, CD59, CD55, and any combinations thereof. In some embodiments, the engineered islets exhibit reduced expression of CD142.

In some embodiments, engineered islets exhibit increased expression of CD47 and reduced expression of B2M. In some embodiments, engineered islets exhibit increased expression of CD47 and reduced expression of CIITA. In some embodiments, the engineered islets exhibit increased expression of CD47 and reduced expression of NLRC5. In some embodiments, the engineered islets exhibit increased expression of CD47 and reduced expression of one or more molecules of B2M and CIITA. In some embodiments, the engineered islets exhibit increased expression of CD47 and reduced expression of one or more molecules of B2M and NLRC5. In some embodiments, the engineered islets exhibit increased expression of CD47 and reduced expression of one or more molecules of B2M, CIITA and NLRC5. Any of the engineered islets described herein can also exhibit increased expression of one or more factors selected from the group including, but not limited to, DUX4, CD24, CD27, CD200, HLA-C, HLA-E, HLA-E heavy chain, HLA-G, PD-L1, IDO1, CTLA4-Ig, C1-Inhibitor, IL-10, IL-35, IL-39, FasL, CCL21, CCL22, Mfge8, and Serpinb9. In some embodiments, the engineered islets express one or more exogenous complement inhibitor polypeptides selected from CD46, CD59, CD55, and any combinations thereof.

In some embodiments, the engineered islets exhibit increased expression of CD47, reduced expression of CD142, and reduced expression of B2M. In some embodiments, the engineered islets exhibit increased expression of CD47, reduced expression of CD142, and reduced expression of CIITA. In some embodiments, the modified beta engineered islets exhibit increased expression of CD47, reduced expression of CD142, and reduced expression of NLRC5. In some embodiments, the engineered islets exhibit increased expression of CD47, reduced expression of CD142, and reduced expression of one or more molecules of B2M and CIITA. In some embodiments, the engineered islets exhibit increased expression of CD47, reduced expression of CD142, and reduced expression of one or more molecules of B2M and NLRC5. In some embodiments, the engineered islets exhibit increased expression of CD47, reduced expression of CD142, and reduced expression of one or more molecules of B2M, CIITA and NLRC5. Any of the engineered islets can also exhibit increased expression of one or more factors selected from the group including, but not limited to, DUX4, CD24, CD27, CD200, HLA-C, HLA-E, HLA-E heavy chain, HLA-G, PD-L1, IDO1, CTLA4-Ig, C1-Inhibitor, IL-10, IL-35, IL-39, FasL, CCL21, CCL22, Mfge8, and Serpinb9. In some embodiments, the engineered islets express one or more exogenous complement inhibitor polypeptides selected from CD46, CD59, CD55, and any combinations thereof.

In some embodiments, the engineered islets exhibit increased expression of CD47 and at least one complement inhibitor selected from the group consisting of CD46, CD59, CD55, and any combination thereof, and reduced expression of one or more molecules of the MHC class I complex. In some embodiments, the engineered islets exhibit increased expression of CD47 and at least one complement inhibitor selected from the group consisting of CD46, CD59, CD55, and any combination thereof, and reduced expression of one or more molecules of the MHC class II complex. In some embodiments, the engineered islets exhibit increased expression of CD47 and at least one complement inhibitor selected from the group consisting of CD46, CD59, CD55, and any combination thereof, and reduced expression of one or more molecules of the MHC class II and MHC class II complexes. In some embodiments, the engineered islets and populations thereof exhibit increased expression of CD47 and at least one complement inhibitor selected from the group consisting of CD46, CD59, CD55, and any combination thereof, and reduced expression of B2M. In some embodiments, the engineered islets and populations thereof exhibit increased expression of CD47 and at least one complement inhibitor selected from the group consisting of CD46, CD59, CD55, and any combination thereof, and reduced expression of CIITA. In some embodiments, the engineered islets and populations thereof exhibit increased expression of CD47 and at least one complement inhibitor selected from the group consisting of CD46, CD59, CD55, and any combination thereof, and reduced expression of NLRC5. In some embodiments, the engineered islets and populations thereof exhibit increased expression of CD47 and at least one complement inhibitor selected from the group consisting of CD46, CD59, CD55, and any combination thereof, and reduced expression of one or more molecules of B2M and CIITA. In some embodiments, the engineered islets and populations thereof exhibit increased expression of CD47 and at least one complement inhibitor selected from the group consisting of CD46, CD59, CD55, and any combination thereof, and reduced expression of one or more molecules of B2M and NLRC5. In some embodiments, the engineered islets and populations thereof exhibit increased expression of CD47 and at least one complement inhibitor selected from the group consisting of CD46, CD59, CD55, and any combination thereof, and reduced expression of one or more molecules of CIITA and NLRC5. In some embodiments, the engineered islets and populations thereof exhibit increased expression of CD47 at least one complement inhibitor selected from the group consisting of CD46, CD59, CD55, and any combination thereof, and reduced expression of one or more molecules of B2M, CIITA and NLRC5.

In some embodiments, the engineered islets and populations thereof exhibit increased expression of CD47 and at least one complement inhibitor selected from the group consisting of CD46, CD59, CD55, and any combination thereof, reduced expression of CD142, and reduced expression of one or more molecules of the MHC class I complex. In some embodiments, the engineered islets and populations thereof exhibit increased expression of CD47 and at least one complement inhibitor selected from the group consisting of CD46, CD59, CD55, and any combination thereof, reduced expression of CD142, and reduced expression of one or more molecules of the MHC class II complex. In some embodiments, the engineered islets and populations thereof exhibit increased expression of CD47 and at least one complement inhibitor selected from the group consisting of CD46, CD59, CD55, and any combination thereof, reduced expression of CD142, and reduced expression of one or more molecules of the MHC class II and MHC class II complexes. In some embodiments, the engineered islets and populations thereof exhibit increased expression of CD47 and at least one complement inhibitor selected from the group consisting of CD46, CD59, CD55, and any combination thereof, reduced expression of CD142, and reduced expression of B2M. In some embodiments, the engineered islets and populations thereof exhibit increased expression of CD47 and at least one complement inhibitor selected from the group consisting of CD46, CD59, CD55, and any combination thereof, reduced expression of CD142, and reduced expression of CIITA. In some embodiments, the engineered islets and populations thereof exhibit increased expression of CD47 and at least one complement inhibitor selected from the group consisting of CD46, CD59, CD55, and any combination thereof, reduced expression of CD142, and reduced expression of NLRC5. In some embodiments, the engineered islets and populations thereof exhibit increased expression of CD47 and at least one complement inhibitor selected from the group consisting of CD46, CD59, CD55, and any combination thereof, reduced expression of CD142, and reduced expression of one or more molecules of B2M and CIITA. In some embodiments, the engineered islets and populations thereof exhibit increased expression of CD47 and at least one complement inhibitor selected from the group consisting of CD46, CD59, CD55, and any combination thereof, reduced expression of CD142, and reduced expression of one or more molecules of B2M and NLRC5. In some embodiments, the engineered islets and populations thereof exhibit increased expression of CD47 and at least one complement inhibitor selected from the group consisting of CD46, CD59, CD55, and any combination thereof, reduced expression of CD142, and reduced expression of one or more molecules of CIITA and NLRC5. In some embodiments, the engineered islets and populations thereof exhibit increased expression of CD47 at least one complement inhibitor selected from the group consisting of CD46, CD59, CD55, and any combination thereof, reduced expression of CD142, and reduced expression of one or more molecules of B2M, CIITA and NLRC5.

In some embodiments, a engineered islets exhibits increased or decreased expression of the one more target molecules (e.g. MHC class I or class II, or CD47) in which the increase or decrease in expression is retained or similar (e.g. 75% to 100% of the level) compared to the unmodified or wild-type cell. Also provided herein is a population of engineered islets that include a plurality of cells that exhibit increased or decreased expression of the one more target molecules (e.g. MHC class I or class II, or CD47).

In some embodiments, at least at or about 50%, at least at or about 60%, at least at or about 70%, at least at or about 80%, or at least at or about 90% of the cells in the population are eliminated for expression of MHC class I or for B2M. In some embodiments, at least at or about 50%, at least at or about 60%, at least at or about 70%, at least at or about 80%, or at least at or about 90% of the cells in the population are eliminated for expression of MHC class II or for CIITA.

In some embodiments, least at or about 50%, at least at or about 60%, at least at or about 70%, at least at or about 80%, or at least at or about 90% of the cells in the population level have increased expression of the tolerogenic factor (CD47) that is greater than at or about 5-fold, greater than at or about 10-fold, greater than at or about 20-fold, greater than at or about 30-fold, greater than at or about 40-fold, greater than at or about 50-fold, greater than at or about 60-fold, or greater than at or about 70-fold over a wild-type primary beta cell or an unmodified pluripotent stem cell or an unmodified SC-beta differentiated from the unmodified pluripotent stem cell. In some embodiments, at least at or about 50%, at least at or about 60%, at least at or about 70%, at least at or about 80%, or at least at or about 90% of the cells in the population expresses the tolerogenic factor (e.g. CD47) at greater than at or about 20,000 molecules per cell, at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

In some embodiments, at least at or about 50%, at least at or about 60%, at least at or about 70%, at least at or about 80%, or at least at or about 90% of the cells in the population are eliminated for expression of CD142.

One skilled in the art will appreciate that levels of expression such as increased or reduced expression of a gene, protein or molecule can be referenced or compared to a comparable cell. In some embodiments, a modified cell (e.g., an engineered islet, such as a modified beta islet cell) having increased expression of a protein (e.g., CD46, CD59. CD55, CD47, or any other tolerogenic factor) refers to a modified cell having a higher level of the protein compared to an unmodified cell. In some embodiments, an engineered islets having increased expression of a protein (e.g., CD46, CD59. CD55, CD47, or any other tolerogenic factor) is a cell comprising modifications, wherein the cell comprising modifications has a higher level of the protein compared to a cell without said modifications (e.g., beta cell without the modifications may comprise other modifications). In some embodiments, an engineered islet having reduced expression of a protein (e.g., CD142, B2M, or CIITA) is a cell comprising modifications, wherein the cell comprising modifications has a lower level of the protein or RNA compared to a cell without said modifications (e.g., beta cell without the modifications may comprise other modifications). In some embodiments, the engineered islets express one or more exogenous complement inhibitor polypeptides selected from CD46, CD59, CD55, and any combinations thereof.

In one embodiment, provided herein are engineered islets expressing exogenous CD47 polypeptides and having reduced expression of CD142 and reduced expression of either one or more MHC class I complex proteins, one or more MHC class II complex proteins, or any combination of MHC class I and class II complex proteins. In another embodiment, the engineered islets express exogenous CD47 polypeptides and express reduced levels CD142 of B2M and CIITA polypeptides. In some embodiments, the engineered islets express exogenous CD47 polypeptides and possess modifications of the CD142, B2M and CIITA genes. In some instances, the modifications inactivate the B2M and CIITA genes. In some embodiments, the engineered islets express one or more exogenous complement inhibitor polypeptides selected from CD46, CD59, CD55, and any combinations thereof.

In one embodiment, provided herein are engineered islets expressing exogenous CD47 polypeptides and having reduced expression of either one or more MHC class I complex proteins, one or more MHC class II complex proteins, or any combination of MHC class I and class II complex proteins. In another embodiment, the engineered islets express exogenous CD47 polypeptides and express reduced levels of B2M and CIITA polypeptides. In some embodiments, the engineered islets express exogenous CD47 polypeptides and possess modifications of the B2M and CIITA genes. In some instances, the modifications inactivate the B2M and CIITA genes. In some embodiments, the modified cells express one or more exogenous complement inhibitor polypeptides selected from CD46, CD59, CD55, and any combinations thereof.

In some embodiments, the engineered islets comprise a CD46 polypeptide having at least 95% sequence identity (e.g., 95%, 96%, 97%, 98%, 99%, or more) to an amino acid sequence as set forth in SEQ ID NO: 3. In some embodiments, the engineered islets comprise a CD46 polypeptide having the amino acid sequence as set forth in SEQ ID NO: 3. In some embodiments, the engineered islets comprise a CD46 polypeptide having at least 95% sequence identity (e.g., 95%, 96%, 97%, 98%, 99%, or more) to an amino acid sequence as set forth in SEQ ID NO: 4. In some embodiments, the engineered islets comprise a CD46 polypeptide having the amino acid sequence as set forth in SEQ ID NO: 4.

In some embodiments, the engineered islets comprise a CD59 polypeptide having at least 95% sequence identity (e.g., 95%, 96%, 97%, 98%, 99%, or more) to an amino acid sequence as set forth in SEQ ID NO: 5. In some embodiments, the engineered islets comprise a CD59 polypeptide having the amino acid sequence as set forth in SEQ ID NO: 5. In some embodiments, the engineered islets comprise a CD59 polypeptide having at least 95% sequence identity (e.g., 95%, 96%, 97%, 98%, 99%, or more) to an amino acid sequence as set forth in SEQ ID NO: 6. In some embodiments, the engineered islets comprise a CD59 polypeptide having the amino acid sequence as set forth in SEQ ID NO: 6. In some embodiments, the engineered islets comprise a CD59 polypeptide having at least 95% sequence identity (e.g., 95%, 96%, 97%, 98%, 99%, or more) to an amino acid sequence as set forth in SEQ ID NO: 7. In some embodiments, the engineered islets comprise a CD59 polypeptide having the amino acid sequence as set forth in SEQ ID NO: 7.

In some embodiments, the engineered islets comprise a CD55 polypeptide having at least 95% sequence identity (e.g., 95%, 96%, 97%, 98%, 99%, or more) to an amino acid sequence as set forth in SEQ ID NO: 8. In some embodiments, the engineered islets comprise a CD55 polypeptide having the amino acid sequence as set forth in SEQ ID NO: 8. In some embodiments, the engineered islets comprise a CD55 polypeptide having at least 95% sequence identity (e.g., 95%, 96%, 97%, 98%, 99%, or more) to an amino acid sequence as set forth in SEQ ID NO: 9. In some embodiments, the engineered islets comprise a CD55 polypeptide having the amino acid sequence as set forth in SEQ ID NO: 9. In some embodiments, the engineered islets comprise a CD55 polypeptide having at least 95% sequence identity (e.g., 95%, 96%, 97%, 98%, 99%, or more) to an amino acid sequence as set forth in SEQ ID NO: 10. In some embodiments, the engineered islets comprise a CD55 polypeptide having the amino acid sequence as set forth in SEQ ID NO: 10.

D. Exemplary Features of the Engineered islets

The engineered islets are highly functional engineered islets. The engineered islets or population exhibits a glucose stimulated insulin secretion (GSIS) response both in vitro and in vivo. The isolated engineered islets or population thereof also exhibits at least one characteristic feature of a mature endogenous beta cell. In some aspects, a engineered islets or population thereof exhibits a stimulation index of between about 1.4 and about 2.4. In some aspects, a engineered islets or population thereof produces insulin at between approximately 300 uIU to about 4000 uIU per 30 minute per 106 total cells incubation at a high glucose concentration.

In certain embodiments, static insulin secretion is greater than about 1 uIU/103 cells/hour at high glucose (e.g. 20 mM). In certain embodiments, the static insulin secretion is greater than about 1.5 uIU/103 cells/hour at high glucose. In certain embodiments, the static insulin secretion is greater than about 2.0 uIU/103 cells/hour at high glucose. In certain embodiments, the static insulin secretion is greater than about 2.5 uIU/103 cells/hour at high glucose. In certain embodiments, the static insulin secretion is greater than about 3.0 uIU/103 cells/hour at high glucose. In certain embodiments, the static insulin secretion is greater than about 3.5 uIU/103 cells/hour at high glucose. In certain embodiments, the static insulin secretion is greater than about 4.0 uIU/103 cells/hour at high glucose. In certain embodiments, the static stimulation index is defined as a ratio of 20 mM glucose to 2 mM glucose, incubated for 1 hr.

Assays to assess functional activity of the engineered islets include static and dynamic GSIS assays to measure glucose responsiveness, insulin content and proinsulin-to-insulin ratio to determine intracellular insulin levels and processing, flow cytometry to measure the percentages of the different hormone-producing cells, and qRT-PCR and immunostaining to confirm the presence of 3-cell-specific markers. In particular, engineered islets can be identified by their coexpression of C-peptide and NKX6-1, while chromogranin A (CHGA) marks the general endocrine population. After aggregation, >80% of the cells in these clusters should express CHGA, with ˜20-60% of these cells being C-peptide+/NKX6-1+.

In certain embodiments, the engineered islets achieve both first and second-phase dynamic insulin secretion. In some embodiments, the engineered islets achieve equivalent functional capabilities of human islets. In certain embodiments, the engineered islets retain functionality for 1 or more days. In certain embodiments, the engineered islets retain functionality for more than 1 week. In certain embodiments, the engineered islets may be used to treat or reverse severe preexisting diabetes at a rate similar to primary human islets, outperforming cells generated with a suspension-based protocol. In certain embodiments, the engineered islets, when transplanted, are capable of maintaining normoglycemia indefinitely.

In some embodiments, the engineered islets comprise engineered beta islet cells. In some embodiments, at least 20%, at least 25%, at least 30%, at least 35%, at least 40%, at least 45%, at least 50%, at least 55%, at least 60%, at least 65%, or at least 70% of the cells in the engineered hypoimmunogenic islets comprise engineered beta islet cells. In some embodiments, at least 20% of the cells in the engineered hypoimmunogenic islets comprise engineered beta islet cells. In some embodiments, at least 25% of the cells in the engineered hypoimmunogenic islets comprise engineered beta islet cells. In some embodiments, at least 30% of the cells in the engineered hypoimmunogenic islets comprise engineered beta islet cells. In some embodiments, at least 35% of the cells in the engineered hypoimmunogenic islets comprise engineered beta islet cells. In some embodiments, at least 40% of the cells in the engineered hypoimmunogenic islets comprise engineered beta islet cells. In some embodiments, at least 45% of the cells in the engineered hypoimmunogenic islets comprise engineered beta islet cells. In some embodiments, at least 50% of the cells in the engineered hypoimmunogenic islets comprise engineered beta islet cells. In some embodiments, at least 55% of the cells in the engineered hypoimmunogenic islets comprise engineered beta islet cells. In some embodiments, at least 60% of the cells in the engineered hypoimmunogenic islets comprise engineered beta islet cells. In some embodiments, at least 65% of the cells in the engineered hypoimmunogenic islets comprise engineered beta islet cells. In some embodiments, at least 70% of the cells in the engineered hypoimmunogenic islets comprise engineered beta islet cells.

1. Assays to Assess Engineered islets Function

A key functional feature of a beta cell is its ability to repeatedly perform glucose stimulated insulin secretion (GSIS). In certain embodiments, assays can be performed to determine the physiological function in vitro of secreting insulin in response to glucose. In certain embodiments, the GSIS assay may be a perfusion GSIS (dynamic GSIS) assay (for example as in Velazco-Cruz, Stem Cell Reports, 2019).

In certain embodiments, other assays can be performed to examine the expression of specific genes, pathways, and transcription factors. Such assays include those detecting the presence of Yap (Rosado-Olivieri et al., 2019), the ROCKII pathway (Ghazizadeh et al., 2017), the transforming growth factor b (TGF-b) pathway (Velazco-Cruz, et al., 2019), the cytoskeleton (Hogrebe et al., 2020), and the expression of SIX2 (Velazco-Cruz et al., Cell Reports, 2020). In certain embodiments, other transcription factors important for the engineered islets phenotype include PDX1, NKX6-1, NKX2-2, and NEUROD1 (Hogrebe et al., 2020).

In certain embodiments, an assay measuring changes in intracellular Ca2+ may be performed as described in Pagliuca et al. (Cell, 2014). Beta cells sense changing glucose levels through calcium signaling; increasing glucose levels leads to membrane depolarization causing an influx of calcium ions which triggers insulin exocytosis (Mohammed et al., 2009). In certain embodiments, the functional engineered islets exhibit calcium flux similarly to primary human islet cells.

In certain embodiments, assays can also be performed to assess in vivo functionality of the engineered islets. An example of such an assay can be found in Pagliuca et al. (Cell, 2014). Briefly, to test their capacity to function in vivo, engineered islets are transplanted under the kidney capsule of immunocompromised mice and the ability of the cells to produce insulin is analyzed.

### E. Assays for Hypoimmunogenic Phenotypes

In some embodiments, the engineered islets are modified such that they are able to evade immune recognition and responses when administered to a patient (e.g. recipient subject). The engineered islets can evade killing by immune cells in vitro and in vivo. In some embodiments, the engineered islets evade killing by macrophages and NK cells. In some embodiments, the engineered islets are ignored by immune cells or a subject's immune system. In other words, the engineered islets administered in accordance with the methods described herein are not detectable by immune cells of the immune system. In some embodiments, the engineered islets are cloaked and therefore avoid immune rejection.

Methods of determining whether a engineered islets provided herein evades immune recognition include, but are not limited to, IFN-γ Elispot assays, microglia killing assays, cell engraftment animal models, cytokine release assays, ELISAs, killing assays using bioluminescence imaging or chromium release assay or Xcelligence analysis, mixed-lymphocyte reactions, immunofluorescence analysis, etc.

In some embodiments, the immunogenicity of the engineered islets is evaluated in a complement-dependent cytotoxicity (CDC) assay. CDC can be assayed in vitro by incubating cells with IgG or IgM antibodies targeting an HLA-independent antigen expressed on the cell surface in the presence of serum containing complement and analyzing cell killing. In some embodiments, CDC can be assayed by incubating cells with ABO blood type incompatible serum, wherein the cells comprise A antigens or B antigens, and the serum comprises antibodies against the A antigens and/or B antigens of the cells.

In some embodiments, once the engineered islets have been modified or generated as described herein, they may be assayed for their hypoimmunogenicity. Any of a variety of assays can be used to assess if the cells are hypoimmunogenic or can evade the immune system. Exemplary assays include any as is described in WO2016183041 and WO2018132783.

In some embodiments, the engineered islets described herein survive in a host without stimulating the host immune response for one week or more (e.g. one week, two weeks, one month, two months, three months, 6 months, one year, two years, three years, four years, five years or more, e.g. for the life of the cell and/or its progeny). The engineered islets maintain expression of the transgenes and/or are deleted or reduced in expression of target genes for as long as they survive in the host. In some aspects, if the transgenes are no longer expressed and/or if target genes are expressed the engineered islets may be removed by the host's immune system. In some embodiments, the persistence or survival of the engineered islets may be monitored after their administration to a recipient by further expressing a transgene encoding a protein that allows the cells to be detected in vivo (e.g. a fluorescent protein, such as GFP, a truncated receptor or other surrogate marker or other detectable marker).

The hypoimmunogenic cells are administered in a manner that permits them to engraft to the intended tissue site and reconstitute or regenerate the functionally deficient area. In some embodiments, the hypoimmunogenic cells are assayed for engraftment (e.g. successful engraftment). In some embodiments, the engraftment of the hypoimmunogenic cells is evaluated after a pre-selected amount of time. In some embodiments, the engrafted cells are monitored for cell survival. For example, the cell survival may be monitored via bioluminescence imaging (BLI), wherein the cells are transduced with a luciferase expression construct for monitoring cell survival. In some embodiments, the engrafted cells are visualized by immunostaining and imaging methods known in the art. In some embodiments, the engrafted cells express known biomarkers that may be detected to determine successful engraftment. For example, flow cytometry may be used to determine the surface expression of particular biomarkers. In some embodiments, the hypoimmunogenic cells are engrafted to the intended tissue site as expected (e.g. successful engraftment of the hypoimmunogenic cells). In some embodiments, the hypoimmunogenic cells are engrafted to the intended tissue site as needed, such as at a site of cellular deficiency. In some embodiments, the hypoimmunogenic cells are engrafted to the intended tissue site in the same manner as a cell of the same type not comprising the modifications.

In some embodiments, administering the combinations (e.g. administering a composition comprising a engineered islets in combination with one or more immunosuppressive agents) improves survival and engraftment by allowing cells to avoid or reduce IBMIR that occurs as a result of exposure of the cells to blood during transplant. In some embodiments, the reduction in IBMIR reduces the amount of cell loss (e.g. loss of transplanted islets) that occurs during transplant.

In some embodiments, the hypoimmunogenic cells are assayed for function. In some embodiments, the hypoimmunogenic cells are assayed for function prior to their engraftment to the intended tissue site. In some embodiments, the hypoimmunogenic cells are assayed for function following engraftment to the intended tissue site. In some embodiments, the function of the hypoimmunogenic cells is evaluated after a pre-selected amount. In some embodiments, the function of the engrafted cells is evaluated by the ability of the cells to produce a detectable phenotype. For example, engrafted beta islet cell function may be evaluated based on the restoration of lost glucose control due to diabetes. In some embodiments, the function of the hypoimmunogenic cells is as expected (e.g. successful function of the hypoimmunogenic cells while avoiding antibody-mediated rejection). In some embodiments, the function of the hypoimmunogenic cells is as needed, such as sufficient function at a site of cellular deficiency while avoiding antibody-mediated rejection. In some embodiments, the engineered islets function in the same manner as a non-modified cell of the same type.

### 1. Instant Blood-Mediated Inflammatory Reaction

In some embodiments, the engineered islets provided herein evade an instant blood-mediated inflammatory reaction. A major contributor to the poor outcome of clinical islet transplantation is the occurrence of the destructive instant blood mediated inflammatory reaction (IBMIR), which leads to loss of transplanted tissue when the islets encounter the blood in the portal vein (Bennet et al., (1995) Diabetes 48:1907-1914; Moberg et al., (2002) Lancet 360:2039-2045). This reaction is triggered by tissue factor (TF) expression by the endocrine cells of the islets, combined with an array of other proinflammatory events, such as the expression of MCP-1 (Piemonti et al., (2002) Diabetes 51:55-65), IL-8, and MIF (Waeber et al., (1997) Proc Natl Acad Sci USA 94:4782-4787; Johansson et al., (2006) Am J Transplantation 6(2):305).

Instant blood-mediated inflammatory reaction (IBMIR) is a nonspecific inflammatory and thrombotic reaction that can occur when cells expressing CD142 come into contact with blood. IBMIR is initiated rapidly by exposure to human blood in the portal vein. It is characterized by activation of complement, platelets, and the coagulation pathway, which in turn leads to the recruitment of neutrophils. IBMIR causes significant loss of transplanted islets. In some embodiments, provided herein are compositions (e.g. modified cells comprising reduced expression of CD142 in combination with one or more of the other modifications described herein), combinations (e.g. a combination comprising any of the populations of modified cells described herein and an anti-coagulant agent that reduces coagulation), and methods (e.g. methods of treating a patient comprising administering any of the populations of modified cells described herein and anti-coagulant agent that reduces coagulation) that reduce an IBMIR associated with transplantation of the cells or exposure of the cells to blood.

In some embodiments, IBMIR can be assayed in vitro, for example, in an in vitro tubing loop model of IBMIR, which has been previously described in U.S. Pat. No. 7,045,502, which is herein incorporated by reference in its entirety.

In some embodiments, IBMIR can be assayed in vivo (e.g. in a mammal or in a human patient) by drawing blood samples during the peritransplant period and evaluating plasma levels of thrombin-anti-thrombin III complex (TAT), C-peptide, factor xIa-antithrombin (FXIa-AT), factor XIIa-antithrombin (FXIIa-AT), thrombin-antithrombin (TAT) plasmin-alpha 2 antiplasmin (PAP), and/or complement C3a. sIn some embodiments, IBMIR is associated with increased levels of TAT, C-peptide, FXIa-AT, FXIIa-AT, PAP, and/or complement C3a during infusion of transplanted cells and/or in a period of time following transplant (e.g. up to 3, 5, 10, or more than 10 hours after transplant). In some embodiments, IBMIR can be assayed by monitoring counts of free circulating platelets, wherein a decrease in the counts of platelets during or following transplantation is associated with IBMIR (e.g. with platelet consumption due to IBMIR).

### 2. Complement Dependent Cytotoxicity

In some embodiments, the engineered islets provided herein evade complement dependent cytotoxicity (CDC). In some embodiments, the CDC is secondary to a thrombotic reaction of IBMIR. In some embodiments, the CDC occurs independently of IBMIR.

In some embodiments, susceptibility of cells to CDC can be analyzed in vitro according to standard protocols understood by one of ordinary skill in the art. In some embodiments, CDC can be analyzed in vitro by mixing serum comprising the components of the complement system (e.g. human serum), with target cells bound by an antibody (e.g. an IgG or IgM antibody), and then to determine cell death. In some embodiments, susceptibility of cells to CDC can be analyzed in vitro by incubating cells in the presence of ABO-incompatible or Rh factor incompatible serum, comprising the components of the complement system and antibodies against ABO type A, ABO type B, and/or Rh factor antigens of the cells.

A common CDC assay determines cell death via pre-loading the target cells with a radioactive compound. As cells die, the radioactive compound is released from them. Hence, the efficacy of the antibody to mediate cell death is determined by the radioactivity level. Unlike radioactive CDC assays, non-radioactive CDC assays often determine the release of abundant cell components, such as GAPDH, with fluorescent or luminescent determination. In some embodiments, cell killing by CDC can be analyzed using a label-free platform such as xCELLigence™ (Agilent).

### IV. Exemplary Embodiments

Among the provided embodiments are:
Embodiment 1. A method of treating or preventing a beta cell disorder in a subject in need thereof, the method comprising administering to the subject a dose of engineered hypoimmunogenic islets,
wherein the dose is administered to the subject via intramuscular injection, and
wherein the dose is a dose from:
   A) about 1×10⁷ cells to about 3×10⁸ cells;
   B) about 1.25×10⁵ cells/kg to about 1.2×10⁷ cells/kg;
   C) about 6,500 islet equivalents (IEQ) to about 600,000 IEQ; or
   D) about 80 IEQ/kg to about 24,000 IEQ/kg.
Embodiment 2. A method of reducing exogenous insulin dependence in a subject having or at risk of having a beta cell disorder, the method comprising administering to the subject a dose of engineered hypoimmunogenic islets, wherein the dose is administered via intramuscular injection, wherein the dose is a dose from:
   A) about 1×10⁷ cells to about 3×10⁸ cells;
   B) about 1.25×10⁵ cells/kg to about 1.2×10⁷ cells/kg;
   C) about 6,500 islet equivalents (IEQ) to about 600,000 IEQ; or
   D) about 80 IEQ/kg to about 24,000 IEQ/kg, and
wherein the amount of exogenous insulin required is less than the amount of exogenous insulin required for a subject treated with non-hypoimmunogenic islets or is less than the amount of exogenous insulin required for untreated subjects that have the beta cell disorder.
Embodiment 3. A method of promoting insulin independence in a subject having or at risk of having a beta cell disorder, the method comprising administering to the subject a dose of engineered hypoimmunogenic islets,
wherein the dose is administered via intramuscular injection, and
wherein the dose is a dose from:
   A) about 1×10⁷ cells to about 3×10⁸ cells;
   B) about 1.25×10⁵ cells/kg to about 1.2×10⁷ cells/kg;
   C) about 6,500 Islet equivalents (IEQ) to about 600,000 IEQ; or
   D) about 80 IEQ/kg to about 24,000 IEQ/kg.
Embodiment 4. A method of improving graft function in a subject having or at risk of having a beta cell disorder, the method comprising administering to the subject a dose of engineered hypoimmunogenic islets,
wherein the dose is administered via intramuscular injection, and
wherein the dose is a dose from:
   A) about 1×10⁷ cells to about 3×10⁷ cells;
   B) about 1.25×10⁵ cells/kg to about 1.2×10⁷ cells/kg;
   C) about 6,500 Islet equivalents (IEQ) to about 600,000 IEQ; or
   D) about 80 IEQ/kg to about 24,000 IEQ/kg.
Embodiment 5. A method of enhancing engraftment in a subject having or at risk of having a beta cell disorder, the method comprising administering to the subject a dose of engineered hypoimmunogenic islets,
wherein the dose is administered via intramuscular injection, and
wherein the dose is a dose from:
   A) about 1×10⁷ cells to about 3×10⁸ cells;
   B) about 1.25×10⁵ cells/kg to about 1.2×10⁷ cells/kg;
   C) about 6,500 Islet equivalents (IEQ) to about 600,000 Islet equivalents (IEQ);
   D) about 80 IEQ/kg to about 24,000 IEQ/kg.
Embodiment 6. A method of stabilizing glucose levels in a subject having or at risk of having a beta cell disorder, the method comprising administering to the subject a dose of engineered hypoimmunogenic islets,
wherein the dose is administered via intramuscular injection,
wherein the dose is a dose from:
   A) about 1×10⁷ cells to about 3×10⁸ cells;
   B) about 1.25×10⁵ cells/kg to about 1.2×10⁷ cells/kg;
   C) about 6,500 islet equivalents (IEQ) to about 600,000 IEQ; or
   D) about 80 IEQ/kg to about 24,000 IEQ/kg,
wherein the glucose levels are stabilized compared to a subject administered an alternative islet therapy or compared to an untreated subject.
Embodiment 7. A method of stabilizing/increasing c-peptide levels in a subject having or at risk of having a beta cell disorder, the method comprising administering to the subject a dose of engineered hypoimmunogenic islets,
wherein the dose is administered via intramuscular injection, and
wherein the dose is a dose from:
   A) about 1×10⁷ cells to about 3×10⁷ cells;
   B) about 1.25×10⁵ cells/kg to about 1.2×10⁷ cells/kg;
   C) about 6,500 islet equivalents (IEQ) to about 600,000 IEQ; or
   D) about 80 IEQ/kg to about 24,000 IEQ/kg,
wherein the c-peptide levels are stabilized or increased compared to a subject administered an alternative islet therapy or compared to an untreated subject.
Embodiment 8. A method of reducing HbA1c levels in a subject having or at risk of having a beta cell disorder, the method comprising administering to the subject a dose of engineered hypoimmunogenic islets,
wherein the dose is administered via intramuscular injection, and
wherein the dose is a dose from:
   A) about 1×10⁷ cells to about 3×10⁸ cells;
   B) about 1.25×10⁵ cells/kg to about 1.2×10⁷ cells/kg;
   C) about 6,500 islet equivalents (IEQ) to about 600,000 IEQ; or
   D) about 80 IEQ/kg to about 24,000 IEQ/kg,
wherein the HbA1c levels are reduced compared to a subject administered an alternative islet therapy or compared to an untreated subject.
Embodiment 9. A method of reducing adverse side effects associated with islet cell therapy in a subject having or at risk of having a beta cell disorder, the method comprising
i) introducing hypoimmunogenic modification to a population of islet cells comprising beta cells to generate engineered hypoimmunogenic islets, and
ii) administering a dose of the engineered hypoimmunogenic islets to a subject having or at risk of having a beta cell disorder,
wherein the dose is administered via intramuscular injection, and
wherein the dose is a dose from:
   A) about 1×10⁷ cells to about 3×10⁷ cells;
   B) about 1.25×10⁵ cells/kg to about 1.2×10⁷ cells/kg;
   C) about 6,500 islet equivalents (IEQ) to about 600,000 IEQ; or
   D) about 80 IEQ/kg to about 24,000 IEQ/kg.
Embodiment 10. A method increasing time in range (TIR) in a subject having or at risk of having a beta cell disorder, the method comprising administering to the subject a dose of engineered hypoimmunogenic islets,
wherein the dose is administered via intramuscular injection, and
wherein the dose is a dose from:
   A) about 1×10⁷ cells to about 3×10⁸ cells;
   B) about 1.25×10⁵ cells/kg to about 1.2×10⁷ cells/kg;
   C) about 6,500 islet equivalents (IEQ) to about 600,000 IEQ; or
   D) about 80 IEQ/kg to about 24,000 IEQ/kg,
wherein the TIR is increased compared to a subject administered an alternative islet therapy or compared to an untreated subject.
Embodiment 11. The method of any of embodiments 1-10, wherein the method results in reduction in other medication requirements for treating the beta cell disorder, optionally wherein the beta cell disorder medication is insulin.
Embodiment 12. The method of any of embodiments 1-11, wherein the subject exhibits reduced insulin dependence.
Embodiment 13. The method of embodiment 2 or embodiment 12, wherein the amount of exogenous insulin is reduced by 10% or more compared to the amount of exogenous insulin required for a subject administered non-hypoimmunogenic islets for treating the beta cell disorder or the amount of exogenous insulin required for untreated subjects that have the beta cell disorder.
Embodiment 14. The method of any of embodiments 2, 12 and 13, wherein the amount of insulin is reduced by more than about 20%, about 30%, about 40%, about 50%, about 60%, about 70%, about 75%, about 80% or more.
Embodiment 15. The method of any of embodiments 2 and 12-14, wherein the method is characterized by the subject meeting one or more of the following criteria: (i) fasting capillary glucose level does not exceed 140 mg/dL (7.8 mmol/L) more than three times in 1 week (based on measuring capillary glucose levels a minimum of 7 times in a seven day period); (ii) 2-hours post-prandial capillary glucose does not exceed 180 mg/dL (10.0 mmol/L) more than three times in 1 week (based on measuring capillary glucose levels a minimum of 21 times in a seven day period); and (iii) evidence of endogenous insulin production defined as fasting or stimulated C-peptide levels >0.5 ng/mL (0.16 pmol/L).
Embodiment 16. The method of any of embodiments 1-9, wherein the method results in the subject exhibiting insulin-independence.
Embodiment 17. The method of embodiment 16, wherein the subject exhibits insulin-independence for a period of greater than one month, greater than two months, greater than three months, greater than four months, greater than 5 months, greater than 6 months, greater than 7 months, greater than 8 months, greater than 9 months, greater than 10 months, greater than 11 months or greater than 12 months.
Embodiment 18. The method of embodiment 17, wherein the subject exhibits insulin-independence for a period of at least 1 year.
Embodiment 19. The method of any of embodiments 1-9 and 16, wherein the subject is able to titrate off insulin therapy for at least 1 week and meet one or more of the following criteria: (i) fasting capillary glucose level does not exceed 140 mg/dL (7.8 mmol/L) more than three times in 1 week (based on measuring capillary glucose levels a minimum of 7 times in a seven day period); (ii) 2-hours post-prandial capillary glucose does not exceed 180 mg/dL (10.0 mmol/L) more than three times in 1 week (based on measuring capillary glucose levels a minimum of 21 times in a seven day period); and (iii) evidence of endogenous insulin production defined as fasting or stimulated C-peptide levels >0.5 ng/mL (0.16 pmol/L).
Embodiment 20. The method of any of embodiments 1-9 and 16-18, wherein the subject is able to titrate off insulin therapy for the period and meet one or more of the following criteria: (i) fasting capillary glucose level does not exceed 140 mg/dL (7.8 mmol/L) more than three times in 1 week (based on measuring capillary glucose levels a minimum of 7 times in a seven day period); (ii) 2-hours post-prandial capillary glucose does not exceed 180 mg/dL (10.0 mmol/L) more than three times in 1 week (based on measuring capillary glucose levels a minimum of 21 times in a seven day period); and (iii) evidence of endogenous insulin production defined as fasting or stimulated C-peptide levels >0.5 ng/mL (0.16 pmol/L).
Embodiment 21. The method of any of embodiments 15, 19 and 20, wherein the subject is characterized by at least two of (i)-(iii).
Embodiment 22. The method of any of embodiments 15, 19 and 20, wherein the subject is characterized by each of (i)-(iii).
Embodiment 23. The method of any of embodiments 1-22, the method is characterized by the subject meeting one or more of the following:
a) Peak c-peptide >0.20 nmol/l (as assessed by mixed meal tolerance test);
b) Non-fasting c-peptide >0.10 nmol/l (as assessed by mixed meal tolerance test);
c) Daily exogenous insulin requirement <0.25 U/kg;
d) Daily exogenous insulin requirement=0 U/kg;
e) Decrease in exogenous insulin requirement (per kg body weight);
f) Decrease in HbA1c (per kg body weight);
g) Decrease in glucose variability (stabilization);
h) Decrease in duration of hypoglycemia and/or hyperglycemia (improved euglycemia);
i) Glycemic control HbA1c<6.5% (48 mmol/mol); and
j) Glycemic control HbA1c≤7.0% (53 mmol/mol).
Embodiment 24. The method of embodiment 23, wherein the method is characterized by the subject meeting 2, 3, 4, 5, 6, 7, 8, 9 or 10 of a)-j).
Embodiment 25. The method of embodiment 23, wherein the method is characterized by the subject meeting each of a)-j).
Embodiment 26. The method of any of embodiments 1-25, wherein the engineered hypoimmunogenic islets comprise modifications that:
(a) inactivate or disrupt one or more alleles of: (i) one or more major histocompatibility complex (MHC) class I molecules or one or more molecules that regulate expression of the one or more MHC class I molecules, and/or (ii) one or more MHC class II molecules or one or more molecules that regulate expression of the one or more MHC class II molecules; and/or
(b) increase expression of one or more tolerogenic factors, wherein the increased expression is relative to a control or wild-type islet that does not comprise the modifications.
Embodiment 27. The method of any one of embodiments 1-26, wherein the engineered hypoimmunogenic islets comprise engineered beta islet cells.
Embodiment 28. The method of embodiment 27, wherein the engineered hypoimmunogenic islets further comprises additional engineered islet cells, optionally wherein the additional engineered islet cells comprise alpha cells and/or delta cells.
Embodiment 29. The method of embodiment 28, wherein the additional engineered islet cells comprises cells that comprises the same modifications of the engineered beta islet cells.
Embodiment 30. The method of any one of embodiments 1-29, wherein at least 20%, at least 25%, at least 30%, at least 35%, at least 40%, at least 45%, at least 50%, at least 55%, at least 60%, at least 65%, or at least 70% of the cells in the engineered hypoimmunogenic islets comprise engineered beta islet cells.
Embodiment 31. The method of embodiment 30, wherein at least 60% of the cells in the engineered hypoimmunogenic islets comprise engineered beta islet cells.
Embodiment 32. The method of any one of embodiments 1-31, wherein the engineered hypoimmunogenic islets is an islet cluster.
Embodiment 33. The method of any one of embodiments 1-32, wherein the engineered hypoimmunogenic islets is engineered from primary islets.
Embodiment 34. The method of embodiment 33, wherein the primary islets are from a pancreas.
Embodiment 35. The method of embodiment 33 or embodiment 34, wherein the primary islets are from a human subject.
Embodiment 36. The method of embodiment 33 or embodiment 34, wherein the primary islets are from an animal subject.
Embodiment 37. The method of embodiment 36, wherein the primary islets are porcine, bovine or ovine.
Embodiment 38. The method of any of embodiments 33-35, wherein the primary islets are from a donor subject that is not suspected of having a beta cell related disorder.
Embodiment 39. The method of embodiment 38, wherein the donor is a cadaver.
Embodiment 40. The method of any one of embodiments 1-39, wherein the engineered hypoimmunogenic islets are ABO blood group type O.
Embodiment 41. The method of any one of embodiments 1-40, wherein the engineered hypoimmunogenic islets are Rhesus factor negative (Rh-).
Embodiment 42. The method of any one of embodiments 1-41, wherein the engineered hypoimmunogenic islets are differentiated from a stem cell.
Embodiment 43. The method of embodiment 42, wherein the stem cell is selected from the group consisting of a pluripotent stem cell (PSC), an induced pluripotent stem cell (iPSC), an embryonic stem cell, a hematopoietic stem cell, a mesenchymal stem cell, an endothelial stem cell, an epithelial stem cell, an adipose stem cell, a germline stem cell, a lung stem cell, a cord blood stem cell, and a multipotent stem cell.
Embodiment 44. The method of embodiment 42 or embodiment 43, wherein the stem cell is an induced pluripotent stem cell (iPSC), mesenchymal stem cell (MSC), hematopoietic stem cell (HSC), or embryonic stem cell (ESC).
Embodiment 45. The method of any of embodiments 42-44, wherein the stem cell is a pluripotent stem cell (PSC).
Embodiment 46. The method of embodiment any of embodiments 1-45, wherein beta cell disorder is a metabolic disorder.
Embodiment 47. The method of embodiment 46, wherein the metabolic disorder is selected from the group consisting of: familial hypercholesterolemia, Gaucher disease, Hunter syndrome, Krabbe disease, maple syrup urine disease, metachromatic leukodystrophy, mitochondrial encephalopathy, lactic acidosis, stroke-like episodes (MELAS), Niemann-Pick disease, phenylketonuria (PKU), porphyria, Tay-Sachs disease, Wilson's disease, Type I diabetes, Type II diabetes, obesity, hypertension, dyslipidemia, and carbohydrate intolerance.
Embodiment 48. The method of any one of embodiments 1-46, wherein the beta cell disorder is diabetes.
Embodiment 49. The method of any of embodiments 1-48, wherein the beta cell disorder is Type I diabetes.
Embodiment 50. The method of any of embodiments 1-49, wherein the subject to be treated is characterized by one or more of the following: type 1 diabetes for more than 5 years, C-peptide negative (or <0.01 nmol/l) in response to mixed meal tolerance test (MMTT), positive for antibodies to either GAD or IA2, HbA_{1c}, >70 mmol/mol, and an exogenous insulin requirement <1 U/kg.
Embodiment 51. The method of any one of embodiments 1-50, wherein the dose of engineered hypoimmunogenic islets comprises a pharmaceutically acceptable carrier.
Embodiment 52. The method of embodiment 51, wherein the pharmaceutically acceptable carrier is a buffered aqueous solution.
Embodiment 53. The method of embodiment 52, wherein the buffered aqueous solution is saline.
Embodiment 54. The method of any one of embodiments 1-53, wherein the dose is administered to the subject intravenously.
Embodiment 55. The method of embodiment 54, wherein intravenous administration is via the portal vein.
Embodiment 56. The method of any one of embodiments 1-53, wherein the dose is administered to the subject via a kidney capsule.
Embodiment 57. The method of any one of embodiments 1-53, wherein the dose is administered to the subject subcutaneously.
Embodiment 58. The method of any one of embodiments 1-53, wherein the dose is administered to the subject intramuscularly.
Embodiment 59. The method of embodiment 58, wherein intramuscular administration is via the intramuscular space of the forearm.
Embodiment 60. The method of any one of embodiments 1-53 and 57-59, wherein the dose is administered to the upper arm, hip, thigh or buttocks.
Embodiment 61. The method of any one of embodiments 1-60, wherein the dose is administered to the liver, kidney, spleen, muscle, subcutaneous tissue or white adipose tissue of the subject.
Embodiment 62. The method of embodiment 61, wherein the dose is administered to the liver, muscle or white adipose tissue of the subject.
Embodiment 63. The method of embodiment 61 or embodiment 62, wherein the white adipose tissue is omentum.
Embodiment 64. The method of any one of embodiments 1-63, wherein the dose comprises administration of one or more further doses of the hypoimmunogenic engineered cells.
Embodiment 65. The method of embodiment 64, wherein the one or more further doses of the hypoimmunogenic engineered cells is administered to the subject when, after the initial dose: (a) the subject does not exhibit a reduction in other medication requirements for treating the beta cell disorder, optionally wherein the beta cell disorder medication is insulin; and/or
(b) the administered hypoimmunogenic engineered cells are not detected by imaging.
Embodiment 66. The method of embodiment 65, wherein the subject does not exhibit reduced insulin dependence after the initial dose.
Embodiment 67. The method of embodiment 64, wherein the one or more further doses of the hypoimmunogenic engineered cells is administered to the subject when, after the initial dose, the subject does not meet one or more of the following criteria: (i) fasting capillary glucose level does not exceed 140 mg/dL (7.8 mmol/L) more than three times in 1 week (based on measuring capillary glucose levels a minimum of 7 times in a seven day period); (ii) 2-hours post-prandial capillary glucose does not exceed 180 mg/dL (10.0 mmol/L) more than three times in 1 week (based on measuring capillary glucose levels a minimum of 21 times in a seven day period); and (iii) evidence of endogenous insulin production defined as fasting or stimulated C-peptide levels >0.5 ng/mL (0.16 pmol/L).
Embodiment 68. The method of embodiment 64, wherein the one or more further doses of the hypoimmunogenic engineered cells is administered to the subject when:
(a) the subject does not achieve insulin-independence within a period of time after the initial dose; and/or
(b) the subject does not exhibit a reduction in other medication requirements for treating the beta cell disorder within a period of time, optionally wherein the beta cell disorder medication is insulin.
Embodiment 69. The method of embodiment 68, wherein the subject does not achieve insulin-independence for a period of greater than one week, greater than two weeks, greater than three weeks, greater than one month, greater than two months, greater than three months, greater than four months, greater than 5 months, greater than 6 months, greater than 7 months, greater than 8 months, greater than 9 months, greater than 10 months, greater than 11 months or greater than 12 months, optionally wherein the subject does not achieve insulin-independence for a period of 2 weeks.
Embodiment 70. The method of embodiment 68 or embodiment 69, wherein the subject does not achieve insulin-independence for a period of at least 1 year.
Embodiment 71. The method of embodiment 64, wherein the one or more further doses of the hypoimmunogenic engineered cells is administered to the subject when, after the initial dose, the subject is not able to titrate off insulin therapy for at least 1 week and meet one or more of the following criteria: (i) fasting capillary glucose level does not exceed 140 mg/dL (7.8 mmol/L) more than three times in 1 week (based on measuring capillary glucose levels a minimum of 7 times in a seven day period); (ii) 2-hours post-prandial capillary glucose does not exceed 180 mg/dL (10.0 mmol/L) more than three times in 1 week (based on measuring capillary glucose levels a minimum of 21 times in a seven day period); and (iii) evidence of endogenous insulin production defined as fasting or stimulated C-peptide levels >0.5 ng/mL (0.16 pmol/L).
Embodiment 72. The method of any one of embodiments 68-70, wherein the one or more further doses of the hypoimmunogenic engineered cells is administered to the subject when, after the initial dose, the subject is not able to titrate off insulin therapy for the period and meet one or more of the following criteria: (i) fasting capillary glucose level does not exceed 140 mg/dL (7.8 mmol/L) more than three times in 1 week (based on measuring capillary glucose levels a minimum of 7 times in a seven day period); (ii) 2-hours post-prandial capillary glucose does not exceed 180 mg/dL (10.0 mmol/L) more than three times in 1 week (based on measuring capillary glucose levels a minimum of 21 times in a seven day period); and (iii) evidence of endogenous insulin production defined as fasting or stimulated C-peptide levels >0.5 ng/mL (0.16 pmol/L).
Embodiment 73. The method of embodiment 71 or embodiment 72, wherein the subject is characterized by not meeting at least two of (i)-(iii) or each of (i)-(iii).
Embodiment 74. The method of embodiment 64, wherein the one or more further doses of the hypoimmunogenic engineered cells is administered to the subject when, after the initial dose, the subject does not meet one or more of the following criteria:
a) Peak c-peptide >0.20 nmol/l (as assessed by mixed meal tolerance test);
b) Non-fasting c-peptide >0.10 nmol/l (as assessed by mixed meal tolerance test);
c) Daily exogenous insulin requirement <0.25 U/kg;
d) Daily exogenous insulin requirement=0 U/kg;
e) Decrease in exogenous insulin requirement (per kg body weight);
f) Decrease in HbA1c (per kg body weight);
g) Decrease in glucose variability (stabilization);
h) Decrease in duration of hypoglycemia and/or hyperglycemia (improved euglycemia);
i) Glycemic control HbA1c<6.50% (48 mmol/mol); and
j) Glycemic control HbA1c≤7.0% (53 mmol/mol).
Embodiment 75. The method of embodiment 74, wherein the one or more further doses is administered to the subject, after the initial dose, if the subject does not meet 2, 3, 4, 5, 6, 7, 8, 9 or 10 of a)-j).
Embodiment 76. The method of embodiment 74, wherein the one or more further doses is administered to the subject, after the initial dose, if the subject does not meet each of a)-j).
Embodiment 77. The method of any one of embodiments 65-76, wherein prior to administering the one or more further doses of engineered hypoimmune islets, the number of the engineered hypoimmunogenic islets from the initial dose are cleared or reduced in the subject.
Embodiment 78. The method of embodiment 77, wherein the number of engineered hypoimmunogenic islets are reduced in the subject following administration of an exogenously administered agent to direct targeted death of the engineered hypoimmunogenic islets.
Embodiment 79. The method of embodiment 78, wherein the exogenously administered agent activates a suicide gene or safety switch in the engineered cells or recognizes one or more tolerogenic factors on the surface of the engineered hypoimmunogenic islets.
Embodiment 80. The method of any one of embodiments 1-79, wherein the subject is administered an immunosuppression regimen.
Embodiment 81. The method of embodiment 80, wherein the immunosuppression regimen comprise one or more of mycophenolate mofetil (MMF), an anti-CD25 antibody (e.g. basiliximab) and a calcineurin inhibitor (e.g., tacrolimus; FK-506).
Embodiment 82. The method of embodiment 81, wherein the immunosuppression regimen comprises administration of Basiliximab (e.g. 2×20 mg iv) followed by Tacrolimus (start dose 0.1 mg/kg/24 h; with target concentration of 10-12 mg/kg/24 h) and MMF immunosuppression (500 mg 2×2, dose adjusted thereafter based on AUC).
Embodiment 83. The method of embodiment 82, wherein the subject is further administered one or more of the following: CMV prophylaxis Valganciclovir (e.g. 450 mg 2×1), an ulcer prophylaxis with omeprazole (e.g. 20 mg 1×1), TNF-alpha inhibition with etanercept (e.g., 50 mg iv, followed by 25 mg sc on day 3, 7 and 10), and standard antibiotics.
Embodiment 84. The method of any one of embodiments 80-84, wherein the immunosuppression regimen is administered to the subject only prior to administration of the dose of the engineered hypoimmunogenic islets.
Embodiment 85. The method of embodiment 84, wherein the immunosuppression regimen is administered to the subject only 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 days prior to administration of the dose of the engineered hypoimmunogenic islets.
Embodiment 86. The method of embodiment 84 or embodiment 85, wherein the immunosuppression regimen is administered to the subject only 1, 2, 3, 4, 5, 6, 7, 8, 9, 10, 11, 12, 13, 14, 15, 16, 17, 18, 19, 20, 21, 22, 23, 24, 25, 26, 27, or 28 days prior to administration of the dose of the engineered hypoimmunogenic islets.
Embodiment 87. The method of any one of embodiments 80-83, wherein the immunosuppression regimen is administered to the subject only 1, 2, 3, 4, or 5 weeks prior to administration of the dose of the engineered hypoimmunogenic islets.
Embodiment 88. The method of embodiment 87, wherein the immunosuppression regimen is administered to the subject only 1, 2, 3, or 4 weeks prior to administration of the dose of the engineered hypoimmunogenic islets.
Embodiment 89. The method of any one of embodiments 80-83, wherein the immunosuppression regimen is administered to the subject only after administration of the dose of the engineered hypoimmunogenic islets.
Embodiment 90. The method of any one of embodiments 80-83, wherein the immunosuppression regimen is administered to the subject only 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 days after administration of the dose of the engineered hypoimmunogenic islets.
Embodiment 91. The method of embodiment 90, wherein the immunosuppression regimen is administered to the subject only 1, 2, 3, 4, 5, 6, 7, 8, 9, 10, 11, 12, 13, 14, 15, 16, 17, 18, 19, 20, 21, 22, 23, 24, 25, 26, 27, or 28 days after administration of the dose of the engineered hypoimmunogenic islets.
Embodiment 92. The method of any one of embodiments 80-83, wherein the immunosuppression regimen is administered to the subject only 1, 2, 3, 4, or 5 weeks after administration of the dose of the engineered hypoimmunogenic islets.
Embodiment 93. The method of embodiment 92, wherein the immunosuppression regimen is administered to the subject only 1, 2, 3, or 4 weeks after administration of the dose of the engineered hypoimmunogenic islets.
Embodiment 94. The method of any one of embodiments 80-93, wherein the immunosuppression regimen is administered to the subject intravenously.
Embodiment 95. The method of any one of embodiments 80-93, wherein the immunosuppression regimen is administered to the subject via a kidney capsule.
Embodiment 96. The method of any one of embodiments 80-93, wherein the immunosuppression regimen is administered to the subject orally.
Embodiment 97. The method of any one of embodiments 80-93, wherein the immunosuppression regimen is administered to the subject rectally.
Embodiment 98. The method of any one of embodiments 80-93, wherein the immunosuppression regimen is administered to the subject subcutaneously.
Embodiment 99. The method of any one of embodiments 80-93, wherein the immunosuppression regimen is administered to the subject intramuscularly.
Embodiment 100. The method of embodiment 99, wherein the immunosuppression regimen is administered to the forearm of the subject.
Embodiment 101. The method of embodiment 94 or embodiment 98, wherein the immunosuppression regimen is administered to the upper arm, hip, thigh or buttocks.
Embodiment 102. The method of any of embodiments 80-101, wherein the immunosuppression regimen is administered at least once daily.
Embodiment 103. The method of any of embodiments 80-101, wherein the immunosuppression regimen is administered as a single regimen per day.
Embodiment 104. The method of any of embodiments 80-101, wherein the immunosuppression regimen is administered as a divided regimen.
Embodiment 105. The method of embodiment 104, wherein the immunosuppression regimen is divided between 2 regimens, 3 regimen.
Embodiment 106. The method of any one of embodiments 80-105, wherein the immunosuppression regimen comprises one or more immunosuppression agents.
Embodiment 107. The method of embodiment 106, wherein the one or more immunosuppression agents are administered to the subject prior to administration of the dose of engineered hypoimmunogenic islets.
Embodiment 108. The method of embodiment 106 or embodiment 107, wherein the one or more immunosuppression agents are administered to the subject only prior to administration of a first and/or second administration of the dose of engineered hypoimmunogenic islets.
Embodiment 109 The method of any one of embodiments 106-108, wherein the one or more immunosuppression agents are administered to the subject at least 1, 2, 3, 4, 5, 6, 7, 8, 9, 10, 11, 12, 13, or 14 days prior to administration of the dose of engineered hypoimmunogenic islets.
Embodiment 110. The method of any one of embodiments 106-108, wherein the one or more immunosuppression agents are administered to the subject at least 1 week, 2 weeks, 3 weeks, 4 weeks, 5 weeks, 6 weeks, 7 weeks, 8 weeks, 9 weeks, 10 weeks or more prior to administration of the dose of engineered hypoimmunogenic islets.
Embodiment 111. The method of any one of embodiments 106-110, wherein the one or more immunosuppression agents are administered to the subject after administration of the dose of engineered hypoimmunogenic islets.
Embodiment 112. The method of any one of embodiments 106-111, wherein the one or more immunosuppression agents are administered to the subject only after administration of a first and/or second administration of the dose of engineered hypoimmunogenic islets.
Embodiment 113. The method of any one of embodiments 106-112, wherein the one or more immunosuppression agents are administered to the subject after administration of the dose of engineered hypoimmunogenic islets.
Embodiment 114. The method of any one of embodiments 106-113, wherein the one or more immunosuppression agents are administered to the subject after administration of the dose of engineered hypoimmunogenic islets.
Embodiment 115. The method of any one of embodiments 106-114, wherein the one or more immunosuppression agents are administered to the subject after administration of the dose of engineered hypoimmunogenic islets.
Embodiment 116. The method of any one of embodiments 106-115, wherein the one or more immunosuppression agents are administered to the subject at least 1, 2, 3, 4, 5, 6, 7, 8, 9, 10, 11, 12, 13, or 14 days after administration of the dose of engineered hypoimmunogenic islets.
Embodiment 117. The method of any one of embodiments 106-116, wherein the one or more immunosuppression agents are administered to the subject at least 1 week, 2 weeks, 3 weeks, 4 weeks, 5 weeks, 6 weeks, 7 weeks, 8 weeks, 9 weeks, 10 weeks, or more, after administration of the dose of engineered hypoimmunogenic islets.
Embodiment 118. The method of embodiment 117, wherein the one or more immunosuppression agents are administered to the subject at least 2 weeks, 4 weeks, 6 weeks, 8 weeks, 10 weeks, 12 weeks, 16 weeks, or more, after administration of the dose of engineered hypoimmunogenic islets.
Embodiment 119. The method of any one of embodiments 106-118, wherein the one or more immunosuppression agents are administered to the subject at least 1 month, 2 months, 3 months, 4 months, 5 months, 6 months, or more, after administration of the dose of engineered hypoimmunogenic islets.
Embodiment 120. The method of any one of embodiments 106-119, wherein the one or more immunosuppression agents are administered to the subject on the same day as the dose of engineered hypoimmunogenic islets.
Embodiment 121. The method of any one of embodiments 106-120, wherein the one or more immunosuppression agents are administered to the subject concurrently with the dose of engineered hypoimmunogenic islets.
Embodiment 122. The method of any one of embodiments 106-110, 120 and 121, wherein the one or more immunosuppression agents are administered to the subject only at least 1, 2, 3, 4, 5, 6, 7, 8, 9, 10, 11, 12, 13, or 14 days prior to administration of a first and/or second administration of the dose of engineered hypoimmunogenic islets.
Embodiment 123. The method of any one of embodiments 106-110, 120 and 121, wherein the one or more immunosuppression agents are administered to the subject only at least 1 week, 2 weeks, 3 weeks, 4 weeks, 5 weeks, 6 weeks, 7 weeks, 8 weeks, 9 weeks, 10 weeks or more prior to administration of a first and/or second administration of the dose of engineered hypoimmunogenic islets.
Embodiment 124. The method of any one of embodiments 106-121, wherein the one or more immunosuppression agents are administered to the subject only at least 1, 2, 3, 4, 5, 6, 7, 8, 9, 10, 11, 12, 13, or 14 days after administration of a first and/or second administration of the dose of engineered hypoimmunogenic islets.
Embodiment 125. The method of any one of embodiments 106-121, wherein the one or more immunosuppression agents are administered to the subject only at least 1 week, 2 weeks, 3 weeks, 4 weeks, 5 weeks, 6 weeks, 7 weeks, 8 weeks, 9 weeks, or 10 weeks after administration of a first and/or second administration of the dose of engineered hypoimmunogenic islets.
Embodiment 126. The method of any one of embodiments 106-121, wherein the one or more immunosuppression agents are administered to the subject only at least 2 weeks, 4 weeks, 6 weeks, 8 weeks, 10 weeks, 12 weeks or 16 weeks after administration of the dose of engineered hypoimmunogenic islets.
Embodiment 127. The method of any one of embodiments 106-121, wherein the one or more immunosuppression agents are administered to the subject only at least 1 month, 2 months, 3 months, 4 months, 5 months or 6 months after administration of the dose of engineered hypoimmunogenic islets.
Embodiment 128. The method of any one of embodiments 106-121, wherein the one or more immunosuppression agents are administered to the subject prior to the administration of the dose of engineered hypoimmunogenic islets, and continued to be administered over the course of the subject's lifespan.
Embodiment 129. The method of any one of embodiments 106-121, wherein the one or more immunosuppression agents are administered to the subject on the same day and/or concurrent with the administration of the dose of engineered hypoimmunogenic islets, and continued to be administered over the course of the subject's lifespan.
Embodiment 130. The method of any one of embodiments 106-121, wherein the one or more immunosuppression agents are administered to the subject after the administration of the dose of engineered hypoimmunogenic islets, and continued to be administered over the course of the subject's lifespan.
Embodiment 131. The method of any one of embodiments 106-121, wherein the one or more immunosuppression agents are administered to the subject prior to each round of the administration the dose of engineered hypoimmunogenic islets, and optionally continued to be administered over the course of the subject's lifespan.
Embodiment 132. The method of any one of embodiments 106-121 and 131, wherein the one or more immunosuppression agents are administered to the subject on the same day and/or concurrent with each round the administration of the dose of engineered hypoimmunogenic islets, and optionally continued to be administered over the course of the subject's lifespan.
Embodiment 133. The method of any one of embodiments 106-121, 131 and 132, wherein the one or more immunosuppression agents are administered to the subject after each round of the administration of the dose of engineered hypoimmunogenic islets, and optionally continued to be administered over the course of the subject's lifespan.
Embodiment 134. The method of any one of embodiments 106-133, wherein the one or more immunosuppression agents are administered to the subject at a lower dosage compared to the dosage of one or more immunosuppressive agents administered the subject to reduce immune rejection of immunogenic cells that do not comprise the modifications of the dose of engineered hypoimmunogenic islets.
Embodiment 135. The method of any one of embodiment 106-134, wherein the one or more immunosuppression agents comprise a small molecule or a biological product.
Embodiment 136. The method of embodiment 135, wherein the biological product is a protein and/or an antibody.
Embodiment 137. The method of embodiment 135, wherein the small molecule is a chemical compound or a nucleic acid.
Embodiment 138. The method of any one of embodiments 106-137, wherein the one or more immunosuppression agents comprise one or more immunomodulatory agents.
Embodiment 139. The method of embodiment 138, wherein the one or more immunomodulatory agents are a small molecule or a biological product.
Embodiment 140. The method of embodiment 139, wherein the biological product is a protein or peptide thereof and/or an antibody.
Embodiment 141. The method of embodiment 139, wherein the small molecule is a chemical compound or a nucleic acid.
Embodiment 142. The method of any one of embodiments 106-141, wherein the one or more immunosuppression agents are a pharmaceutical salt thereof, a preform thereof and/or a derivative thereof.
Embodiment 143. The method of any one of embodiments 138-141, wherein the one or more immunomodulatory agents are a pharmaceutical salt thereof, a preform thereof and/or a derivative thereof.
Embodiment 144. The method of any one of embodiments 106-143, wherein the one or more immunosuppression agents are selected from the group consisting of calcineurin inhibitors, steroids, alkylating agents, antibiotics, analgesics, anti-inflammatory agents, antihistamines, antiviral agents, anti-fungal agents, anti-coagulation agents, DNA synthesis inhibitors, anti-coagulation agents, hemorheologic agents, inosine monophosphate dehydrogenase (IMDH) inhibitors, Janus kinase inhibitors, mTOR inhibitors, TNF inhibitors, and anti-CD25 inhibitors Embodiment 145. The method of embodiment 144, wherein the one or more immunosuppression agents are selected from the group consisting of antithymocyte globulin (ATG), corticosteroids, prednisone, cortisone, prednisolone methylprednisolone, dexamethasone, betamethasone, hydrocortisone, methotrexate, acetaminophen, diphenhydramine, sirolimus (rapamycin), tacrolimus (FK-506), mycophenolic acid (MPA), mycophenolate mofetil (MMF), mycophenolate sodium, cyclosporine, etanercept (TNFR-Fc), azathioprine, gold salts, sulfasalazine, antimalarials, brequinar, leflunomide, mizoribine, 15-deoxyspergualine, 6-mercaptopurine, cyclophosphamide, OKT3, anti-thymocyte globulin, thymopentin (thymosin-α), fludarabine, and an immunosuppressive antibody.
Embodiment 146. The method of any one of embodiments 106-145, wherein the one or more immunosuppression agents comprise antithymocyte globulin (ATG).
Embodiment 147. The method of embodiment 146, wherein at least one regimen of ATG is administered to the subject prior to, concurrent with, and/or after the administration of the dose of engineered hypoimmunogenic islets to the subject.
Embodiment 148. The method of embodiment 147, wherein the at least one regimen of ATG is administered to the subject prior to the administration of the dose of engineered hypoimmunogenic islets to the subject.
Embodiment 149. The method of embodiment 147, wherein the at least one regimen of ATG is administered to the subject prior to each administration of the dose of engineered hypoimmunogenic islets to the subject.
Embodiment 150. The method of 147, wherein the at least one regimen of ATG is administered to the subject about 7 days prior to, about 6 days prior to, about 5 days prior to, about 4 days prior to, about 3 days prior to, about 2 days prior to, about 1 day prior to, about 12 hours prior to, about 10 hours prior to, about 8 hours prior to, about 6 hours prior to, about 4 hours prior to, about 2 hours prior to, or about 1 hour prior to the administration of the dose of engineered hypoimmunogenic islets to the subject.
Embodiment 151. The method of any one of embodiments 147-150, wherein the at least one regimen of ATG is administered to the subject about 2 days prior and/or about 1 day prior to the administration of the dose of engineered hypoimmunogenic islets to the subject.
Embodiment 152. The method of any one of embodiments 147-150, wherein the at least one regimen of ATG is administered to the subject on the same day and/or concurrent with the administration of the dose of engineered hypoimmunogenic islets to the subject.
Embodiment 153. The method of any one of embodiments 147-150, wherein the at least one regimen of ATG is administered to the subject on the same day and/or concurrent with each administration of the dose of engineered hypoimmunogenic islets to the subject.
Embodiment 154. The method of any one of embodiments 147-150, wherein the at least one regimen of ATG is administered to the subject after the administration of the dose of engineered hypoimmunogenic islets to the subject.
Embodiment 155. The method of embodiment 154, wherein the at least one regimen of ATG is administered to the subject after each administration of the dose of engineered hypoimmunogenic islets to the subject.
Embodiment 156. The method of embodiment 154, wherein the at least one regimen of ATG is administered to the subject about 1 hour after, about 2 hours after, about 4 hours after, about 6 hours after, about 8 hours after, about 10 hours after, about 12 hours, about 24 hours subsequent, or about 48 hours after the administration of the dose of engineered hypoimmunogenic islets to the subject.
Embodiment 157. The method of embodiment 154 or embodiment 156, wherein the at least one regimen of ATG is administered to the subject about 48 hours after the administration of the dose of engineered hypoimmunogenic islets to the subject.
Embodiment 158. The method of any one of embodiments 147-157, wherein the at least one regimen of ATG is administered to the subject: i) about 2 days prior; ii) about 1 day prior; iii) on the same day; iv) about 1 day after; and/or, v) about 2 days after the administration of the dose of engineered hypoimmunogenic islets to the subject.
Embodiment 159. The method of any one of embodiments 147-158, wherein at least two regimens of ATG are administered to the subject prior to, concurrent with, concurrent with, and/or after the administration of the dose of engineered hypoimmunogenic islets to the subject.
Embodiment 160. The method of any one of embodiments 147-159, wherein the at least one or at least two regimens of ATG comprise between about 0.1 mg/kg and about 2.0 mg/kg ATG.
Embodiment 161. The method of embodiment 160, wherein the ATG regimen is administered at a lower dose.
Embodiment 162. The method of any one of embodiments 147-159, wherein:
i) the at least one or at least two regimens of ATG comprise a dose of about 0.5 mg/kg of ATG administered to the subject about 2 days prior to the administration of the dose of engineered hypoimmunogenic islets to the subject;
ii) the at least one or at least two regimens of ATG comprise a dose of about 1.0 mg/kg of ATG administered to the subject about 1 day prior to the administration of the dose of engineered hypoimmunogenic islets to the subject; and/or,
iii) the at least one or at least two regimens of ATG comprise a dose of about 1.5 mg/kg of ATG administered to the subject on the same day as the administration of the dose of engineered hypoimmunogenic islets to the subject, about 1 day after the administration of the dose of engineered hypoimmunogenic islets to the subject, and about 2 days after the administration of the dose of engineered hypoimmunogenic islets to the subject.
Embodiment 163. The method of embodiment 162, wherein the ATG regimen is administered at a lower dose.
Embodiment 164. The method of any one of embodiments 106-162, wherein the one or more immunosuppression agents comprise a corticosteroid.
Embodiment 165. The method of any one of embodiments 106-164, wherein the one or more immunosuppression agents comprise prednisone, cortisone, prednisolone methylprednisolone, dexamethasone, betamethasone, or hydrocortisone.
Embodiment 166. The method of any one of embodiments 106-165, wherein the one or more immunosuppression agents comprise methylprednisolone.
Embodiment 167. The method of embodiment 166, wherein at least one regimen of methylprednisolone is administered to the subject prior to the administration of the dose of engineered hypoimmunogenic islets to the subject.
Embodiment 168. The method of embodiment 167, wherein the at least one regimen of methylprednisolone is administered to the subject about 7 days prior to, about 6 days prior to, about 5 days prior to, about 4 days prior to, about 3 days prior to, about 2 days prior to, about 1 day prior to, about 12 hours prior to, about 10 hours prior to, about 8 hours prior to, about 6 hours prior to, about 4 hours prior to, about 2 hours prior to, or about 1 hour prior to the administration of the dose of engineered hypoimmunogenic islets to the subject.
Embodiment 169. The method of any one of embodiments 166-168, wherein the at least one regimen methylprednisolone is administered to the subject about 2 days prior to the administration of the dose of engineered hypoimmunogenic islets to the subject.
Embodiment 170. The method of any one of embodiments 166-169, wherein the at least one regimenmethylprednisolone is administered to the subject prior to the administration of a first regimen of ATG to the subject, wherein both the regimen of methylprednisolone and the first regimen of ATG are administered to the subject prior to the administration of the dose of engineered hypoimmunogenic islets to the subject.
Embodiment 171. The method of any one of embodiments 166-170, wherein the at least one regimenmethylprednisolone is administered to the subject about 1 hour prior to the administration of a first regimen of ATG to the subject.
Embodiment 172. The method of any one of embodiments 166-170, wherein the at least one regimen methylprednisolone is administered to the subject about midway through the administration of a first regimen of ATG to the subject.
Embodiment 173. The method of any one of embodiments 166-172, wherein the at least one regimen methylprednisolone comprises a dose between about 0.1 mg/kg and about 2.0 mg/kg.
Embodiment 174. The method of embodiment 173, wherein the methylprednisolone regimen is administered at a lower dose.
Embodiment 175. The method of embodiment 173, wherein the at least one regimen of methylprednisolone comprises a dose of about 1.0 mg/kg of methylprednisolone.
Embodiment 176. The method of embodiment 174, wherein the methylprednisolone regimen is administered at a lower dose.
Embodiment 177. The method of any one of embodiments 166-174, wherein the methylprednisolone is administered to the subject intravenously.
Embodiment 178. The method of any one of embodiments 166-176, wherein:
i) the at least one regimen of methylprednisolone comprises about 1.0 mg/kg of methylprednisolone administered to the subject about 1 hour prior to the administration of a first regimen of ATG to the subject; and/or
ii) the at least one regimen of methylprednisolone comprises about 1.0 mg/kg of methylprednisolone administered to the subject about midway through the administration of a first regimen of ATG to the subject.
Embodiment 179. The method of embodiment 178, wherein the methylprednisolone regimen and/or the ATG regimen is administered at a lower dose.
Embodiment 180. The method of any one of embodiments 106-178, wherein the one or more immunosuppression agents comprise an analgesic.
Embodiment 181. The method of embodiment 180, wherein the analgesic is acetaminophen, an opioid, or a non-steroidal anti-inflammatory drug (NSAID).
Embodiment 182. The method of embodiment 180, wherein at least one regimen of acetaminophen is administered to the subject prior to the administration of the dose of engineered hypoimmunogenic islets to the subject.
Embodiment 183. The method of embodiment 182, wherein the at least one regimen of acetaminophen is administered to the subject about 7 days prior to, about 6 days prior to, about 5 days prior to, about 4 days prior to, about 3 days prior to, about 2 days prior to, about 1 day prior to, about 12 hours prior to, about 10 hours prior to, about 8 hours prior to, about 6 hours prior to, about 4 hours prior to, about 2 hours prior to, or about 1 hour prior to the administration of the dose of engineered hypoimmunogenic islets to the subject.
Embodiment 184. The method of any one of embodiments 180-183, wherein the at least one regimen of acetaminophen is administered to the subject about 2 days prior to the administration of the dose of engineered hypoimmunogenic islets to the subject.
Embodiment 185. The method of any one of embodiments 180-183, wherein the at least one regimen of acetaminophen is administered to the subject prior to the administration of a first regimen of ATG to the subject, wherein both the regimen of acetaminophen and the first regimen of ATG are administered to the subject prior to the administration of the dose of engineered hypoimmunogenic islets to the subject.
Embodiment 186. The method of any one of embodiments 180-185, wherein the at least one regimen of acetaminophen is administered to the subject about 30 minutes prior to the administration of a first regimen of ATG to the subject.
Embodiment 187. The method of any one of embodiments 180-185, wherein the at least one regimen of acetaminophen is administered to the subject about midway through the administration of a first regimen of ATG to the subject.
Embodiment 188. The method of any one of embodiments 180-187, wherein the at least one regimen of acetaminophen comprises between about 100 mg and about 1,000 mg of acetaminophen is administered to the subject.
Embodiment 189. The method of embodiment 188, wherein the acetaminophen regimen is administered at a lower dose.
Embodiment 190. The method of embodiment 188, wherein the at least one regimen of acetaminophen comprises about 650 mg of acetaminophen administered to the subject.
Embodiment 191. The method of embodiment 190, wherein the acetaminophen regimen is administered at a lower dose.
Embodiment 192. The method of any one of embodiments 180-190, wherein the acetaminophen is administered to the subject orally or rectally.
Embodiment 193. The method of any one of embodiments 180-192, wherein:
i) the at least one regimen of acetaminophen comprises about 650 mg of acetaminophen administered to the subject about 30 minutes prior to the administration of a first regimen ATG to the subject; and/or
ii) the at least one regimen of acetaminophen comprises about 650 mg of acetaminophen administered to the subject about midway through the administration of the first regimen ATG to the subject.
Embodiment 194. The method of embodiment 193, wherein the acetaminophen regimen and/or the ATG regimen is administered at a lower dose.
Embodiment 195. The method of any one of embodiments 106-193, wherein the one or more immunosuppression agents comprise an antihistamine.
Embodiment 196. The method of embodiment 195, wherein the antihistamine is diphenhydramine.
Embodiment 197. The method of embodiment 195, wherein at least one regimen of diphenhydramine is administered to the subject prior to the administration of the dose of engineered hypoimmunogenic islets to the subject.
Embodiment 198. The method of embodiment 197, wherein the at least one regimen of diphenhydramine is administered to the subject about 7 days prior to, about 6 days prior to, about 5 days prior to, about 4 days prior to, about 3 days prior to, about 2 days prior to, about 1 day prior to, about 12 hours prior to, about 10 hours prior to, about 8 hours prior to, about 6 hours prior to, about 4 hours prior to, about 2 hours prior to, or about 1 hour prior to the administration of the dose of engineered hypoimmunogenic islets to the subject.
Embodiment 199. The method of any one of embodiments 196-198, wherein the at least one regimen of diphenhydramine is administered to the subject about 2 days prior to the administration of the dose of engineered hypoimmunogenic islets to the subject.
Embodiment 200. The method of any one of embodiments 196-199, wherein the at least one regimen of diphenhydramine is administered to the subject prior to the administration of a first regimen of ATG to the subject, wherein both the regimen of diphenhydramine and the first regimen of ATG are administered to the subject prior to the administration of a dose of engineered hypoimmunogenic islets to the subject.
Embodiment 201. The method of any one of embodiments 196-200, wherein the at least one regimen of diphenhydramine is administered to the subject about 30 minutes prior to the administration of a first regimen of ATG to the subject.
Embodiment 202. The method of any one of embodiments 196-199, wherein the at least one regimen of diphenhydramine is administered to the subject about midway through the administration of a first regimen of ATG to the subject.
Embodiment 203. The method of any one of embodiments 196-202, wherein the at least one regimen of diphenhydramine comprises between about 10 mg and about 100 mg of diphenhydramine is administered to the subject.
Embodiment 204. The method of embodiment 203, wherein the diphenhydramine regimen is administered at a lower dose.
Embodiment 205. The method of embodiment 203, wherein the at least one regimen of about 50 mg of diphenhydramine is administered to the subject.
Embodiment 206. The method of embodiment 205, wherein the diphenhydramine regimen is administered at a lower dose.
Embodiment 207. The method of any one of embodiments 196-205, wherein the diphenhydramine is administered to the subject orally or rectally.
Embodiment 208. The method of any one of embodiments 196-207, wherein:
i) the at least one regimen of diphenhydramine comprises about 50 mg of a diphenhydramine administered to the subject about 30 minutes prior to the administration of a first regimen ATG to the subject; and/or
ii) the at least one regimen of diphenhydramine comprises about 50 mg of diphenhydramine administered to the subject about midway through the administration of the first regimen ATG to the subject.
Embodiment 209. The method of embodiment 208, wherein the diphenhydramine regimen and/or the ATG regimen is administered at a lower dose.
Embodiment 210. The method of any one of embodiments 106-208, the one or more immunosuppression agents comprise an anti-inflammatory agent.
Embodiment 211. The method of embodiment 210, wherein the anti-inflammatory agent is a TNF inhibitor.
Embodiment 212. The method of embodiment 210, wherein the TNF inhibitor is selected from the group consisting of infliximab, adalimumab, etanercept, golimumab, and certolizumab.
Embodiment 213. The method of embodiment 211, wherein the TNF inhibitor is etanercept (TNFR-Fc).
Embodiment 214. The method of embodiment 210, wherein at least one regimen of etanercept is administered to the subject prior, concurrent with, and/or after the administration of the dose of engineered hypoimmunogenic islets to the subject.
Embodiment 215. The method of embodiment 214, wherein the at least one regimen of etanercept is administered to the subject prior to the administration of the dose of engineered hypoimmunogenic islets to the subject.
Embodiment 216. The method of embodiment 215, wherein the at least one regimen of etanercept is administered to the subject about 7 days prior to, about 6 days prior to, about 5 days prior to, about 4 days prior to, about 3 days prior to, about 2 days prior to, about 1 day prior to, about 12 hours prior to, about 10 hours prior to, about 8 hours prior to, about 6 hours prior to, about 4 hours prior to, about 2 hours prior to, or about 1 hour prior to the administration of the dose of engineered hypoimmunogenic islets to the subject.
Embodiment 217. The method of any one of embodiments 210-215, wherein the at least one regimen of etanercept is administered to the subject on the same day and/or concurrent with the administration of the dose of engineered hypoimmunogenic islets to the subject.
Embodiment 218. The method of any one of embodiments 210-217, wherein the at least one regimen of etanercept is administered to the subject on the same day and/or concurrent with each administration of the dose of engineered hypoimmunogenic islets to the subject.
Embodiment 219. The method of any one of embodiments 210-218, wherein the at least one regimen of etanercept is administered to the subject after the administration of the dose of engineered hypoimmunogenic islets to the subject.
Embodiment 220. The method of embodiment 219, wherein the at least one regimen of etanercept is administered to the subject about 1 hour after, about 5 hours after, about 10 hours after, about 24 hours after, about 2 days after, about 3 days after, about 5 days, about 7 days subsequent, or about 10 days after the administration of the dose of engineered hypoimmunogenic islets to the subject.
Embodiment 221. The method of embodiment 219 or embodiment 220, wherein the at least one regimen of etanercept is administered to the subject about 3 days, about 7 days, and/or about 10 days after the administration of the dose of engineered hypoimmunogenic islets to the subject.
Embodiment 222. The method of any one of embodiments 210-221, wherein the at least one regimen of etanercept is administered to the subject: i) on the same day; ii) about 3 days subsequent; iii) about 7 days subsequent; and/or iv) about 10 days subsequent, to the administration of the dose of engineered hypoimmunogenic islets to the subject.
Embodiment 223. The method of any one of embodiments 210-222, wherein the at least one regimen of etanercept comprises between about 10 mg and about 100 mg.
Embodiment 224. The method of embodiment 223, wherein the etanercept regimen is administered at a lower dose.
Embodiment 225. The method of embodiment 223, wherein the at least one regimen of etanercept comprises about 50 mg.
Embodiment 226. The method of embodiment 225, wherein the etanercept regimen is administered at a lower dose.
Embodiment 227. The method of embodiment 223, wherein the at least one regimen of etanercept comprises about 25 mg of etanercept.
Embodiment 228. The method of embodiment 227, wherein the etanercept regimen is administered at a lower dose.
Embodiment 229. The method of any one of embodiments 210-227, wherein the etanercept is administered to the subject intravenously and/or subcutaneously.
Embodiment 230. The method of any one of embodiments 210-229, wherein:
i) the at least one regimen of etanercept comprises about 50 mg of etanercept administered to the subject on the same day as the administration of the dose of engineered hypoimmunogenic islets to the subject; and/or,
ii) the at least one regimen of etanercept comprises about 25 mg etanercept administered to the subject about 3 days, about 7 days, and/or about 10 days after the administration of the dose of engineered hypoimmunogenic islets to the subject.
Embodiment 231. The method of embodiment 230, wherein the etanercept regimen is administered at a lower dose.
Embodiment 232. The method of any one of embodiments 210-230, wherein the subject is administered at least one regimen of etanercept and at least one regimen of ATG.
Embodiment 233. The method of embodiment 232, wherein the subject is administered the at least one regimen of ATG prior to the at least one regimen of etanercept.
Embodiment 234. The method of embodiment 232 or embodiment 233, wherein:
   i) the at least one regimen of ATG comprises about 40 mg/kg of ATG mg administered to the subject each day for four consecutive days;
   ii) the at least one regimen of etanercept comprises about 25 mg of etanercept administered to the subject twice a week for two consecutive weeks after i); and,
   iii) the at least one regimen of etanercept comprises about 25 mg of etanercept administered to the subject once a month for about four months after ii).
Embodiment 235. The method of embodiment 234, wherein the at least one etanercept regimen and/or the at least one ATG regimen is administered at a lower dose.
Embodiment 236. The method of any one of embodiments 210-232, wherein the subject is administered at least one regimen of etanercept and at least one regimen of an IL-1 receptor antagonist.
Embodiment 237. The method of any one of embodiments 106-233, wherein the one or more immunosuppression agents comprise an mTOR inhibitor.
Embodiment 238. The method of embodiment 237, wherein the mTOR inhibitor is sirolimus (rapamycin).
Embodiment 239. The method of embodiment 238, wherein at least one regimen of sirolimus is administered to the subject prior, concurrent with, and/or after the administration of the dose of engineered hypoimmunogenic islets to the subject.
Embodiment 240. The method of embodiment 237 or embodiment 238, wherein at least one regimen of sirolimus is administered to the subject prior to the administration of the dose of engineered hypoimmunogenic islets to the subject.
Embodiment 241. The method of embodiment 240, wherein the at least one regimen of sirolimus is administered to the subject about 7 days prior to, about 6 days prior to, about 5 days prior to, about 4 days prior to, about 3 days prior to, about 2 days prior to, about 1 day prior to, about 12 hours prior to, about 10 hours prior to, about 8 hours prior to, about 6 hours prior to, about 4 hours prior to, about 2 hours prior to, or about 1 hour prior to the administration of the dose of engineered hypoimmunogenic islets to the subject.
Embodiment 242. The method of any one of embodiments 238-241, wherein at least one regimen of sirolimus is administered to the subject on the same day and/or concurrent with the administration of the dose of engineered hypoimmunogenic islets to the subject.
Embodiment 243. The method of any one of embodiments 238-242, wherein at least one regimen of sirolimus is administered to the subject after the administration of the dose of engineered hypoimmunogenic islets to the subject.
Embodiment 244. The method of embodiment 243, wherein the at least one regimen of sirolimus is administered to the subject about 1 hour after, about 5 hours after, about 10 hours after, or about 24 hours after, about 3 months after, about 6 months after, about 12 months after, about 24 months after, about 36 months after, about 48 months after, about 60 months after, or more, the administration of the dose of engineered hypoimmunogenic islets to the subject.
Embodiment 245. The method of any one of embodiments 238-244, wherein a total daily dosage of sirolimus administered to the subject yields a blood trough level of between about 1 ng/mL and about 30 ng/mL, between about 2 ng/mL and about 25 ng/mL, between about 5 ng/mL and about 20 ng/mL, or between about 10 ng/mL and about 15 ng/mL, inclusive of each.
Embodiment 246. The method of any one of embodiments 238-245, wherein a regimen of between about 0.1 mg/kg and about 0.2 mg/kg of sirolimus is administered to the subject.
Embodiment 247. The method of embodiment 245 or embodiment 246, wherein the sirolimus regimen is administered at a lower dose.
Embodiment 248. The method of any one of embodiments 238-246, wherein the sirolimus is administered to the subject orally.
Embodiment 249. The method of any one of embodiments 238-248, wherein:
   i) a regimen of about 0.2 mg/kg of sirolimus is administered to the subject on the same day as the administration of the dose of engineered hypoimmunogenic islets to the subject;
   ii) a regimen of about 0/1 mg/kg of sirolimus is administered to the subject each day up to about 3 months after the administration of the dose of engineered hypoimmunogenic islets to the subject,
   wherein the total daily dosage of sirolimus administered to the subject yields a blood trough level of between about 12 ng/mL and about 15 ng/mL for about 3 months after the administration of the composition and between about 7 ng/mL and about 10 ng/mL thereafter.
Embodiment 250. The method of embodiment 249, wherein the sirolimus regimen is administered at a lower dose.
Embodiment 251. The method of any one of embodiments 106-250, wherein the one or more immunosuppression agents comprise a calcineurin inhibitor.
Embodiment 252. The method of embodiment 251, wherein the calcineurin inhibitor is tacrolimus (FK-506).
Embodiment 253. The method of embodiment 251, wherein at least one regimen of tacrolimus is administered to the subject prior, concurrent with, and/or after the administration of the dose of engineered hypoimmunogenic islets to the subject.
Embodiment 254. The method of embodiment 251 or embodiment 253, wherein at least one regimen of tacrolimus is administered to the subject prior to the administration of the dose of engineered hypoimmunogenic islets to the subject.
Embodiment 255. The method of embodiment 254, wherein the at least one regimen of tacrolimus is administered to the subject about 7 days prior to, about 6 days prior to, about 5 days prior to, about 4 days prior to, about 3 days prior to, about 2 days prior to, about 1 day prior to, about 12 hours prior to, about 10 hours prior to, about 8 hours prior to, about 6 hours prior to, about 4 hours prior to, about 2 hours prior to, or about 1 hour prior to the administration of the dose of engineered hypoimmunogenic islets to the subject.
Embodiment 256. The method of any one of embodiments 251-255, wherein at least one regimen of tacrolimus is administered to the subject on the same day and/or concurrent with the administration of the dose of engineered hypoimmunogenic islets to the subject.
Embodiment 257. The method of any one of embodiments 251-255, wherein a first regimen of tacrolimus is administered to the subject on the same day and/or concurrent with the administration of the dose of engineered hypoimmunogenic islets to the subject.
Embodiment 258. The method of any one of embodiments 251-257, wherein at least one regimen of tacrolimus is administered to the subject after the administration of the dose of engineered hypoimmunogenic islets to the subject.
Embodiment 259. The method of embodiment 258, wherein the at least one regimen of tacrolimus is administered to the subject about 1 hour after, about 5 hours after, about 10 hours after, or about 24 hours after, about 3 months after, about 6 months after, about 12 months after, about 24 months after, about 36 months after, about 48 months after, about 60 months after, or more, the administration of the dose of engineered hypoimmunogenic islets to the subject.
Embodiment 260. The method of any one of embodiments 251-259, wherein the total daily dosage of tacrolimus administered to the subject yields a blood trough level of between about 1 ng/mL and about 30 ng/mL, between about 2 ng/mL and about 25 ng/mL, between about 5 ng/mL and about 20 ng/mL, or between about 10 ng/mL and about 15 ng/mL, inclusive of each.
Embodiment 261. The method of any one of embodiments 251-260, wherein the total daily dosage of tacrolimus administered to the subject yields a blood trough level of between about 5 ng/mL and about 10 ng/mL, inclusive of each.
Embodiment 262. The method of any one of embodiments 251-260, wherein the total daily dosage of tacrolimus administered to the subject yields a blood trough level of between about 10 ng/mL and about 15 ng/mL, inclusive of each.
Embodiment 263. The method of any one of embodiments 251-262, wherein a regimen of between about 0.1 mg and about 5 mg of tacrolimus is administered to the subject.
Embodiment 264. The method of any one of embodiments 251-263, wherein the tacrolimus regimen is administered at a lower dose.
Embodiment 265. The method of any one of embodiments 106-263, wherein the one or more immunosuppression agents comprise an inosine-”-monophosphate dehydrogenase (IMPDH) inhibitor.
Embodiment 266. The method of embodiment 264, wherein the IMPDH inhibitor is MPA, MMF, or MS.
Embodiment 267. The method of embodiment 265, wherein the IMPDH inhibitor is mycophenolic acid (MPA).
Embodiment 268. The method of embodiment 267, wherein at least one regimen of MPA is administered to the subject prior, concurrent with, and/or after the administration of the dose of engineered hypoimmunogenic islets to the subject.
Embodiment 269. The method of embodiment 267 or embodiment 268, wherein at least one regimen of MPA is administered to the subject prior to the administration of the dose of engineered hypoimmunogenic islets to the subject.
Embodiment 270. The method of embodiment 269, wherein the at least one regimen of MPA is administered to the subject about 7 days prior to, about 6 days prior to, about 5 days prior to, about 4 days prior to, about 3 days prior to, about 2 days prior to, about 1 day prior to, about 12 hours prior to, about 10 hours prior to, about 8 hours prior to, about 6 hours prior to, about 4 hours prior to, about 2 hours prior to, or about 1 hour prior to the administration of the dose of engineered hypoimmunogenic islets to the subject.
Embodiment 271. The method of any one of embodiments 267-270, wherein at least one regimen of MPA is administered to the subject on the same day and/or concurrent with the administration of the dose of engineered hypoimmunogenic islets to the subject.
Embodiment 272. The method of any one of embodiments 267-271, wherein a first regimen of MPA is administered to the subject on the same day and/or concurrent with the administration of the dose of engineered hypoimmunogenic islets to the subject.
Embodiment 273. The method of any one of embodiments 267-272, wherein at least one regimen of MPA is administered to the subject after the administration of the dose of engineered hypoimmunogenic islets to the subject.
Embodiment 274. The method of embodiment 273, wherein the at least one regimen of MPA is administered to the subject about 1 hour after, about 5 hours after, about 10 hours after, or about 24 hours after, about 3 months after, about 6 months after, about 12 months after, about 24 months after, about 36 months after, about 48 months after, about 60 months after, or more, the administration of the dose of engineered hypoimmunogenic islets to the subject.
Embodiment 275. The method of any one of embodiments 267-274, wherein the MPA is mycophenolate mofetil (MMF).
Embodiment 276. The method of embodiment 275, wherein the total daily dosage of MMF is between about 10 mg and about 3000 mg, about 500 mg and about 3000 mg, between about 1000 mg and about 2500 mg, or between about 1500 mg and about 2000 mg, inclusive of each.
Embodiment 277. The method of embodiment 275 or embodiment 276, wherein the total daily dosage of MMF is about 100 mg, 500 mg, 1000 mg, about 1500 mg, about 2000 mg, or about 2500 mg.
Embodiment 278. The method embodiment 276 or embodiment 277, wherein the total daily dosage of MMF is lower.
Embodiment 279. The method of any one of embodiments 267-274, wherein the MPA is mycophenolate sodium (MS).
Embodiment 280. The method of embodiment 279, wherein the total daily dosage of MS is between about 10 mg and about 2700 mg, about 360 mg and about 2700 mg, between about 720 mg and about 2160 mg, or between about 720 mg and about 1620 mg, inclusive of each.
Embodiment 281. The method of embodiment 279 or embodiment 280, wherein the total daily dosage of MS is about 100 mg, about 360 mg, about 720 mg, about 1080 mg, or about 1440 mg.
Embodiment 282. The method of embodiment 280 or embodiment 281, wherein the total daily dosage of MS is lower.
Embodiment 283. The method of any one of embodiments 252-281, wherein the subject is administered at least one regimen of tacrolimus and at least one regimen of MPA.
Embodiment 284. The method of any one of embodiments 106-283, wherein the one or more immunosuppression agents comprise cyclosporine.
Embodiment 285. The method of embodiment 284, wherein at least one regimen of cyclosporine is administered to the subject prior, concurrent with, and/or after the administration of the dose of engineered hypoimmunogenic islets to the subject.
Embodiment 286. The method of embodiment 285, wherein at least one regimen of cyclosporine is administered to the subject when the subject displays intolerance to a regimen of tacrolimus.
Embodiment 287. The method of embodiment 286, wherein at least one regimen of cyclosporine is administered to the subject on the same day and/or concurrent with the administration of the dose of engineered hypoimmunogenic islets to the subject.
Embodiment 288. The method of any one of embodiments 284-287, wherein a first regimen of cyclosporine is administered to the subject on the same day and/or concurrent with the administration of the dose of engineered hypoimmunogenic islets to the subject.
Embodiment 289. The method of any one of embodiments 284-288, wherein at least one regimen of cyclosporine is administered to the subject after the administration of the dose of engineered hypoimmunogenic islets to the subject.
Embodiment 290. The method of embodiment 284, wherein the at least one regimen of cyclosporine is administered to the subject about 1 hour after, about 5 hours after, about 10 hours after, or about 24 hours after, about 3 months after, about 6 months after, about 12 months after, about 24 months after, about 36 months after, about 48 months after, about 60 months after, or more, the administration of the dose of engineered hypoimmunogenic islets to the subject.
Embodiment 291. The method of any one of embodiments 284-290, wherein the total daily dosage of cyclosporine administered to the subject yields a blood trough level of between about 50 ng/mL and about 300 ng/mL, between about 100 ng/mL and about 250 ng/mL, between about 200 ng/mL and about 300 ng/mL, or between about 150 ng/mL and about 200 ng/mL, inclusive of each.
Embodiment 292. The method of any one of embodiments 284-291, wherein a regimen of between about 2 mg/kg and about 10 mg/kg of cyclosporine is administered to the subject each day.
Embodiment 293. The method of embodiment 292, wherein the cyclosporine regimen is administered at a lower dose.
Embodiment 294. The method of embodiment 292, wherein a regimen of about 6 mg/kg of cyclosporine is administered to the subject each day.
Embodiment 295. The method of embodiment 294, wherein the cyclosporine regimen is administered at a lower dose.
Embodiment 296. The method of any one of embodiments 284-294, wherein the subject is administered at least one regimen of cyclosporine and at least one regimen of MPA.
Embodiment 297. The method of any one of embodiments 284-294, wherein the subject is administered at least one regimen of cyclosporine and at least one regimen of ATG.
Embodiment 298. The method of embodiment 297, wherein the subject is administered the at least one regimen of ATG prior to the at least one regimen of cyclosporine.
Embodiment 299. The method of embodiment 297 or embodiment 298, wherein:
   i) a regimen of about 40 mg/kg of ATG mg is administered to the subject each day for four consecutive days; and,
   ii) a regimen of between about 10 mg/kg and about 12 mg/kg of cyclosporine is administered to the subject each day for six months after i).
Embodiment 300. The method of embodiment 299, wherein the cyclosporine regimen and/or the ATG regimen is administered at a lower dose.
Embodiment 301. The method of any one of embodiments 106-296, wherein the one or more immunosuppression agents comprise an antibody for binding to MHC, CD2, CD3, CD4, CD7, CD28, B7, CD25, CD40, CD45, CD95, IFN-gamma, TNF-alpha, IL-2Ralpha, IL-4, IL-5, IL-6R, IL-6, IGF, IGFR1, IL-7, IL-8, IL-10, CD1lalpha, or CD58, and antibodies binding to any of their ligands.
Embodiment 302. The method of any one of embodiments 106-301, wherein the one or more immunosuppression agents comprise soluble IL-15R, IL-10, B7 molecules such as B7-1, B7-2, variants thereof, and fragments thereof, ICOS, and OX40.
Embodiment 303. The method of any one of embodiments 106-302, wherein the one or more immunosuppression agents comprise an inhibitor of a negative T cell regulator, such as an antibody against CTLA-4, or similar agents.
Embodiment 304. The method of any one of embodiments 106-303, wherein the one or more immunosuppression agents comprise an anti-CD25 antibody or an anti-IL-2Ralpha antibody.
Embodiment 305. The method of embodiment 304, wherein the anti-CD25 antibody or the anti-IL-2Ralpha antibody is selected from the group consisting of basiliximab, daclizumab, and alemtuzumab.
Embodiment 306. The method of embodiment 305, wherein the one or more immunosuppression agents comprise basiliximab.
Embodiment 307. The method of embodiment 306, wherein at least one regimen of basiliximab is administered to the subject on the same day, concurrent with, and/or after the administration of the dose of engineered hypoimmunogenic islets to the subject.
Embodiment 308. The method of embodiment 307, wherein the at least one regimen of basiliximab is administered to the subject about 1 hour after, about 5 hours after, about 10 hours after, or about 24 hours after, about 3 months after, about 6 months after, about 12 months after, about 24 months after, about 36 months after, about 48 months after, about 60 months after, or more, the administration of the dose of engineered hypoimmunogenic islets to the subject.
Embodiment 309. The method of any one of embodiments 306-308, wherein at least one regimen of basiliximab is administered to the subject after the administration of at least one regimen of ATG to the subject.
Embodiment 310. The method of any one of embodiments 306-309, wherein at least one of basiliximab is administered to the subject after the administration of at least one regimen of ATG and after the administration of the dose of engineered hypoimmunogenic islets to the subject.
Embodiment 311. The method of any one of embodiments 306-310, wherein at least one regimen of basiliximab is administered to the subject about 4 days after the administration of the dose of engineered hypoimmunogenic islets to the subject.
Embodiment 312. The method of any one of embodiments 306-311, wherein a regimen of between about 10 mg and about 30 mg of basiliximab is administered to the subject.
Embodiment 313. The method of embodiment 312, wherein a regimen of between about 20 mg of basiliximab is administered to the subject.
Embodiment 314. The method of embodiment 313, wherein the basiliximab regimen is administered at a lower dose.
Embodiment 315. The method of any one of embodiments 306-313, wherein;
   i) a regimen of about 20 mg of basiliximab is administered to the subject on the same day as the administration of the dose of engineered hypoimmunogenic islets to the subject; and/or,
   ii) a regimen of about 20 mg of basiliximab is administered to the subject about 4 days after the administration of the dose of engineered hypoimmunogenic islets to the subject.
Embodiment 316. The method of embodiment 315, wherein the one or more immunosuppression agents comprise daclizumab.
Embodiment 317. The method of embodiment 316, wherein at least one regimen of daclizumab is administered to the subject on the same day, concurrent with, and/or after the administration of the dose of engineered hypoimmunogenic islets to the subject.
Embodiment 318. The method of embodiment 317, wherein the at least one regimen of daclizumab is administered to the subject about 1 hour after, about 5 hours after, about 10 hours after, or about 24 hours after, about 3 months after, about 6 months after, about 12 months after, about 24 months after, about 36 months after, about 48 months after, about 60 months after, or more, the administration of the dose of engineered hypoimmunogenic islets to the subject.
Embodiment 319. The method of any one of embodiments 316-318, wherein the at least one regimen of daclizumab is administered to the subject about every 14 days after the administration of the dose of engineered hypoimmunogenic islets to the subject.
Embodiment 320. The method of any one of embodiments 316-319, wherein a regimen of between about 0.5 mg/kg and about 2 mg/kg of daclizumab is administered to the subject.
Embodiment 321. The method of embodiment 320, wherein the daclizumab regimen is administered at a lower dose.
Embodiment 322. The method of embodiment 320, wherein a regimen of about 1 mg/kg of daclizumab is administered to the subject.
Embodiment 323. The method of embodiment 322, wherein the daclizumab regimen is administered at a lower dose.
Embodiment 324. The method of any one of embodiments 284-322, wherein the subject is administered at least one regimen of tacrolimus and at least one regimen of sirolimus.
Embodiment 325. The method of any one of embodiments 284-322, wherein the subject is administered at least one regimen of tacrolimus and at least one regimen of daclizumab.
Embodiment 326. The method of any one of embodiments 284-322, wherein the subject is administered at least one regimen of sirolimus and at least one regimen of daclizumab.
Embodiment 327. The method of any one of embodiments 284-322, wherein the subject is administered at least one regimen of tacrolimus, at least one regimen of sirolimus, and at least one regimen of daclizumab.
Embodiment 328. The method of embodiment 327 wherein:
   i) a regimen of about 0.2 mg/kg of sirolimus is administered to the subject on the same day as the administration of the dose of engineered hypoimmunogenic islets to the subject;
   ii) a regimen of about 0.1 mg/kg of sirolimus is administered to the subject each day after the administration of the dose of engineered hypoimmunogenic islets to the subject,
      wherein the total daily dosage of sirolimus administered to the subject yields a blood trough level of between about 12 ng/mL and about 15 ng/mL, inclusive of each, for the first three months after the administration of the composition to the subject, and wherein the total daily dosage of sirolimus administered to the subject yields a blood trough level of between about 7 ng/mL and about 10 ng/mL, inclusive of each, after the first three months;
   iii) a regimen of about 1 mg of tacrolimus is administered to the subject on the same day as the administration of the dose of engineered hypoimmunogenic islets to the subject;
   iv) a regimen of about 1 mg of tacrolimus is administered to the subject twice a day about 12 hours after the administration of the dose of engineered hypoimmunogenic islets to the subject,
      wherein the total daily dosage of tacrolimus administered to the subject yields a blood trough level of between about 3 ng/mL and about 6 ng/mL, inclusive of each; and/or
   v) a regimen of about 1 mg/kg of daclizumab is administered to the subject about every 14 days after the administration of the dose of engineered hypoimmunogenic islets to the subject.
Embodiment 329. The method of embodiment 328, wherein the sirolimus regimen, tacrolimus regimen, and or the daclizumab regimen is administered at a lower dose.
Embodiment 330. The method of embodiment 328 or embodiment 329, wherein the subject is not administered glucocorticoids.
Embodiment 331. The method of embodiment 305, wherein the one or more immunosuppression agents comprise alemtuzumab.
Embodiment 332. The method of embodiment 331, wherein at least one regimen of alemtuzumab is administered to the subject prior to, on the same day, concurrent with, and/or after the administration of the dose of engineered hypoimmunogenic islets to the subject.
Embodiment 333. The method of embodiment 331 or embodiment 332, wherein at least one regimen of alemtuzumab is administered to the subject before at least one regimen of tacrolimus and/or MPA is administered to the subject.
Embodiment 334. The method of embodiment 333, wherein the at least one regimen of alemtuzumab and the at least one regimen of tacrolimus and/or MPA is administered to the subject after the administration of the dose of engineered hypoimmunogenic islets of the subject.
Embodiment 335. The method of any one of embodiments 106-334, wherein the one or more immunosuppression agents comprise an anti-CD3 antibody.
Embodiment 336. The method of embodiment 335, wherein the anti-CD3 antibody is an anti-CD3E antibody.
Embodiment 337. The method of embodiment 335, wherein the anti-CD3 antibody is OKT3.
Embodiment 338. The method of any one of embodiments 106-337, wherein the one or more immunosuppression agents comprise an anti-IL-33 antibody.
Embodiment 339. The method of any one of embodiments 106-338, wherein the one or more immunosuppression agents comprise an anti-CD95 antibody.
Embodiment 340. The method of any one of embodiments 106-339, wherein the one or more immunosuppression agents comprise fingolimod hydrochloride.
Embodiment 341. The method of any one of embodiments 106-340, wherein the one or more immunosuppression agents comprise liposomal clodronate.
Embodiment 342. The method of any one of embodiments 106-341, wherein the one or more immunosuppression agents comprise CTLA4-Ig.
Embodiment 343. The method of any one of embodiments 106-342, wherein the one or more immunosuppression agents comprise aryl hydrocarbon receptor (AhR) ligand 2-(1′H-indole-3′-carbonyl)-thiazole-4-carboxylic acid methyl ester (ITE).
Embodiment 344. The method of any one of embodiments 106-343, wherein the one or more immunosuppression agents comprise T1D autoantigen proinsulin.
Embodiment 345. The method of any one of embodiments 106-344, wherein the one or more immunosuppression agents comprise TGF-β1.
Embodiment 346. The method of any one of embodiments 106-345, wherein the one or more immunosuppression agents comprise dexamethasone.
Embodiment 347. The method of any one of embodiments 106-346, wherein the one or more immunosuppression agents comprise methotrexate.
Embodiment 348. The method of any one of embodiments 106-347, wherein the one or more immunosuppression agents comprise gold salts.
Embodiment 349. The method of any one of embodiments 106-348, wherein the one or more immunosuppression agents comprise sulfasalazine.
Embodiment 350. The method of any one of embodiments 106-349, wherein the one or more immunosuppression agents comprise one or more anti-malarials.
Embodiment 351. The method of any one of embodiments 106-350, wherein the one or more immunosuppression agents comprise brequinar.
Embodiment 352. The method of any one of embodiments 106-351, wherein the one or more immunosuppression agents comprise leflunomide.
Embodiment 353. The method of any one of embodiments 106-352, wherein the one or more immunosuppression agents comprise mizoribine.
Embodiment 354. The method of any one of embodiments 106-353, wherein the one or more immunosuppression agents comprise 15-deoxyspergualine.
Embodiment 355. The method of any one of embodiments 106-354, wherein the one or more immunosuppression agents comprise 6-mercaptopurine.
Embodiment 356. The method of any one of embodiments 106-355, wherein the one or more immunosuppression agents comprise cyclophosphamide.
Embodiment 357. The method of any one of embodiments 106-356, wherein the one or more immunosuppression agents comprise anti-thymocyte globulin.
Embodiment 358. The method of any one of embodiments 106-357, wherein the one or more immunosuppression agents comprise an antibiotic agent.
Embodiment 359. The method of embodiment 358, wherein the antibiotic agent is selected from the group consisting of trimethoprim/sulfamethoxaxole, penicillin, amoxicillin, cephalexin, erythromycin (E-Mycin), clarithromycin (Biaxin), azithromycin (Zithromax), ciprofolxacin (Cipro), levofloxacin (Levaquin), ofloxacin (Floxin), co-trimoxazole (Bactrim) and trimethoprim (Proloprim), tetracycline (Sumycin, Panmycin) and doxycycline (Vibramycin), gentamicin (Garamycin), and tobramycin (Tobrex).
Embodiment 360. The method of embodiment 358, wherein the antibiotic agent is trimethoprim/sulfamethoxaxole.
Embodiment 361. The method of embodiment 360, wherein at least one regimen of trimethoprim/sulfamethoxaxole is administered to the subject after the administration of the dose of engineered hypoimmunogenic islets to the subject.
Embodiment 362. The method of embodiment 361, wherein the at least one regimen of trimethoprim/sulfamethoxaxole is administered to the subject about 1 hour after, about 5 hours after, about 10 hours after, or about 24 hours after, about 3 months after, about 6 months after, about 12 months after, about 24 months after, about 36 months after, about 48 months after, about 60 months after, or more, the administration of the dose of engineered hypoimmunogenic islets to the subject.
Embodiment 363. The method of embodiment 362, wherein the at least one regimen of trimethoprim/sulfamethoxaxole is administered to the subject every day for about 6 months after the administration of the dose of engineered hypoimmunogenic islets to the subject.
Embodiment 364. The method of any one of embodiments 360-363, wherein a regimen of between about 50 mg and about 500 mg trimethoprim/sulfamethoxaxole is administered to the subject.
Embodiment 365. The method of embodiment 364, wherein the trimethoprim/sulfamethoxaxole regimen is administered at a lower dose.
Embodiment 366. The method of embodiment 364, wherein a regimen of between about 80 mg and about 400 mg trimethoprim/sulfamethoxaxole is administered to the subject.
Embodiment 367. The method of embodiment 366, wherein the trimethoprim/sulfamethoxaxole regimen is administered at a lower dose.
Embodiment 368. The method of any one of embodiments 106-367, wherein the one or more immunosuppression agents comprise an antifungal agent.
Embodiment 369. The method of embodiment 368, wherein the antifungal agent is selected from the group consisting of clotrimazole, miconazole, ketoconazole, itraconazole, and fluconazole.
Embodiment 370. The method of embodiment 368, wherein the antifungal agent is clotrimazole.
Embodiment 371. The method of embodiment 370, wherein at least one regimen of clotrimazole is administered to the subject prior to, on the same day, concurrent with, and/or after the administration of the dose of engineered hypoimmunogenic islets to the subject.
Embodiment 372. The method of embodiment 370 or embodiment 371, wherein a regimen of clotrimazole is administered to the subject about four times each day.
Embodiment 373. The method of any one of embodiments 370-372, wherein at least one regimen of clotrimazole is administered to the subject each day up to about three months after the administration of the dose of engineered hypoimmunogenic islets to the subject Embodiment 374. The method of any one of embodiments 106-373, wherein the one or more immunosuppression agents comprise an antiviral agent.
Embodiment 375. The method of embodiment 374, wherein the antiviral agent is selected from the group consisting of darunavir, atazanavir, ritonavir, acyclovir, valacyclovir, valganciclovir, tenofovir, and raltegravir.
Embodiment 376. The method of embodiment 374, wherein the antiviral agent is an anti-cytomegaloviral agent.
Embodiment 377. The method of embodiment 374 or embodiment 375, wherein the antiviral agent is valganciclovir.
Embodiment 378. The method of embodiment 377, wherein at least one regimen of valganciclovir is administered to the subject after the administration of the dose of engineered hypoimmunogenic islets to the subject.
Embodiment 379. The method of embodiment 378, wherein the at least one regimen of valganciclovir is administered to the subject about 1 hour after, about 5 hours after, about 10 hours after, or about 24 hours after, about 3 months after, about 6 months after, about 12 months after, about 24 months after, about 36 months after, about 48 months after, about 60 months after, or more, the administration of the dose of engineered hypoimmunogenic islets to the subject.
Embodiment 380. The method of any one of embodiments 377-379, wherein a regimen of between about 300 mg and about 1,000 mg valganciclovir is administered to the subject.
Embodiment 381. The method of embodiment 380, wherein the valganciclovir regimen is administered at a lower dose.
Embodiment 382. The method of embodiment 380, wherein a regimen of about 450 mg valganciclovir is administered to the subject each day after the administration of the dose of engineered hypoimmunogenic islets to the subject.
Embodiment 383. The method of embodiment 382, wherein the valganciclovir regimen is administered at a lower dose.
Embodiment 384. The method of embodiment 380, wherein a regimen of about 900 mg valganciclovir is administered to the subject each day after about day 12 after the administration of the dose of engineered hypoimmunogenic islets to the subject.
Embodiment 385. The method of embodiment 384, wherein the valganciclovir regimen is administered at a lower dose.
Embodiment 386. The method of embodiment 381, wherein the regimen of 900 mg valganciclovir is administered to the subject through about week 14 after the administration of the dose of engineered hypoimmunogenic islets to the subject.
Embodiment 387. The method of any one of embodiments 106-386, wherein the one or more immunosuppression agents comprise a hemorheologic agent.
Embodiment 388. The method of any embodiment 387, wherein the hemorheologic agent is pentoxifylline.
Embodiment 389. The method of embodiment 388, wherein at least one regimen of pentoxifylline is administered to the subject prior to, on the same day as, concurrent with, and/or after the administration of the dose of engineered hypoimmunogenic islets to the subject.
Embodiment 390. The method of embodiment 389, wherein at least one regimen of pentoxifylline is administered to the subject prior to the administration of the dose of engineered hypoimmunogenic islets to the subject.
Embodiment 391. The method of embodiment 390, wherein the at least one regimen of pentoxifylline is administered to the subject about 7 days prior to, about 6 days prior to, about 5 days prior to, about 4 days prior to, about 3 days prior to, about 2 days prior to, about 1 day prior to, about 12 hours prior to, about 10 hours prior to, about 8 hours prior to, about 6 hours prior to, about 4 hours prior to, about 2 hours prior to, or about 1 hour prior to the administration of the dose of engineered hypoimmunogenic islets to the subject.
Embodiment 392. The method of embodiment 391, wherein the at least one regimen of pentoxifylline is administered to the subject about 2 days prior to the administration of the dose of engineered hypoimmunogenic islets to the subject.
Embodiment 393. The method of any one of embodiments 289-392, wherein at least one regimen of pentoxifylline is administered to the subject after the administration of the dose of engineered hypoimmunogenic islets to the subject.
Embodiment 394. The method of embodiment 393, wherein the at least one regimen of pentoxifylline is administered to the subject about 1 hour after, about 5 hours after, about 10 hours after, or about 24 hours after, about 3 months after, about 6 months after, about 12 months after, about 24 months after, about 36 months after, about 48 months after, about 60 months after, or more, the administration of the dose of engineered hypoimmunogenic islets to the subject.
Embodiment 395. The method of embodiment 393 or embodiment 394, wherein at least one regimen of pentoxifylline is administered to the subject through about day 7 after the administration of the dose of engineered hypoimmunogenic islets to the subject.
Embodiment 396. The method of any one of embodiments 389-395, wherein a regimen of between about 300 mg and about 500 mg of pentoxifylline is administered to the subject.
Embodiment 397. The method of embodiment 396, wherein the pentoxifylline regimen is administered at a lower dose.
Embodiment 398. The method of any one of embodiments 106-396, wherein the one or more immunosuppression agents comprise one or more anticoagulation agents.
Embodiment 399. The method of embodiment 398, wherein the one or more anticoagulation agents are selected from the group consisting of aspirin, enoxaparin, and heparin.
Embodiment 400. The method of embodiment 399, wherein the one or more anticoagulation agents is aspirin.
Embodiment 401. The method of embodiment 400, wherein at least one regimen of aspirin is administered to the subject after the administration of the dose of engineered hypoimmunogenic islets to the subject.
Embodiment 402. The method of embodiment 399, wherein the one or more anticoagulation agents is enoxaparin.
Embodiment 403. The method of embodiment 402, wherein at least one regimen of enoxaparin is administered to the subject after the administration of the dose of engineered hypoimmunogenic islets to the subject.
Embodiment 404. The method of embodiment 399, wherein the one or more anticoagulation agents is heparin.
Embodiment 405. The method of embodiment 404, wherein at least one regimen of heparin is administered to the subject after the administration of the dose of engineered hypoimmunogenic islets to the subject.
Embodiment 406. The method of any one of embodiments 402-405, wherein at least one regimen of enoxaparin is administered to the subject after the administration of at least one regimen of heparin to the subject.
Embodiment 407. The method any one of embodiments 106-406, wherein the one or more immunosuppression agents comprise a DNA synthesis inhibitor.
Embodiment 408. The method of embodiment 407, wherein the DNA synthesis inhibitor is fludarabine.
Embodiment 409. The method of embodiment 408, wherein at least one regimen of fludarabine is administered to the subject prior to, concurrent with, and/or after the administration of the dose of engineered hypoimmunogenic islets to the subject.
Embodiment 410. The method of embodiment 408 or embodiment 409, wherein at least one regimen of fludarabine is administered to the subject prior to the administration of the dose of engineered hypoimmunogenic islets to the subject.
Embodiment 411. The method of embodiment 410, wherein the at least one regimen of fludarabine is administered to the subject about 14 days prior to, about 10 days prior to, 7 days prior to, about 6 days prior to, about 5 days prior to, about 4 days prior to, about 3 days prior to, about 2 days prior to, about 1 day prior to, about 12 hours prior to, about 10 hours prior to, about 8 hours prior to, about 6 hours prior to, about 4 hours prior to, about 2 hours prior to, or about 1 hour prior to the administration of the dose of engineered hypoimmunogenic islets to the subject.
Embodiment 412. The method of any one of embodiments 408-411, wherein a first regimen of fludarabine is administered to the subject about 2 days to about 14 days prior to the administration of the dose of engineered hypoimmunogenic islets to the subject.
Embodiment 413. The method of any one of embodiments 408-412, wherein a regimen of fludarabine is administered to the subject each day for about 2 days, about 3 days, or about 4 days prior to the administration of the dose of engineered hypoimmunogenic islets to the subject.
Embodiment 414. The method of any one of embodiments 408-412, wherein a regimen of fludarabine is administered to the subject on day 5, day 4, and day 3 prior to the administration of the dose of engineered hypoimmunogenic islets to the subject.
Embodiment 415. The method of any one of embodiments 408-414, wherein a regimen of between about 10 mg/m² and about 40 mg/m² of fludarabine is administered to the subject.
Embodiment 416. The method of embodiment 415, wherein the fludarabine regimen is administered at a lower dose.
Embodiment 417. The method of embodiment 415 or embodiment 416, wherein a regimen of about 30 mg/m² of fludarabine is administered to the subject.
Embodiment 418. The method of embodiment 417, wherein the fludarabine regimen is administered at a lower dose.
Embodiment 419. The method of any one of embodiments 408-418, wherein fludarabine is administered to the subject intravenously.
Embodiment 420. The method of embodiment 106, wherein the one or more immunosuppression agents comprise an alkylating agent.
Embodiment 421. The method of embodiment 420, wherein the alkylating agent is cyclophosphamide.
Embodiment 422. The method of embodiment 421, wherein at least one regimen of cyclophosphamide is administered to the subject prior to, concurrent with, and/or after the administration of the dose of engineered hypoimmunogenic islets to the subject.
Embodiment 423. The method of embodiment 421 or embodiment 422, wherein at least one regimen of cyclophosphamide is administered to the subject prior to the administration of the dose of engineered hypoimmunogenic islets to the subject.
Embodiment 424. The method of embodiment 423, wherein the at least one regimen of cyclophosphamide is administered to the subject about 14 days prior to, about 10 days prior to, 7 days prior to, about 6 days prior to, about 5 days prior to, about 4 days prior to, about 3 days prior to, about 2 days prior to, about 1 day prior to, about 12 hours prior to, about 10 hours prior to, about 8 hours prior to, about 6 hours prior to, about 4 hours prior to, about 2 hours prior to, or about 1 hour prior to the administration of the dose of engineered hypoimmunogenic islets to the subject.
Embodiment 425. The method of any one of embodiments 421-424, wherein a first regimen of cyclophosphamide is administered to the subject about 2 days to about 14 days prior to the administration of the dose of engineered hypoimmunogenic islets to the subject.
Embodiment 426. The method of any one of embodiments 421-425, wherein a regimen of cyclophosphamide is administered to the subject each day for about 2 days, about 3 days, or about 4 days prior to the administration of the dose of engineered hypoimmunogenic islets to the subject.
Embodiment 427. The method of any one of embodiments 421-425, wherein a regimen of cyclophosphamide is administered to the subject on day 5, day 4, and day 3 prior to the administration of the dose of engineered hypoimmunogenic islets to the subject.
Embodiment 428. The method of any one of embodiments 421-427, wherein a regimen of between about 400 mg/m² and about 600 mg/m² of cyclophosphamide is administered to the subject.
Embodiment 429. The method of embodiment 428, wherein the cyclophosphamide regimen is administered at a lower dose.
Embodiment 430. The method of embodiment 428, wherein a regimen of about 500 mg/m² of cyclophosphamide is administered to the subject.
Embodiment 431. The method of embodiment 430, wherein the cyclophosphamide regimen is administered at a lower dose.
Embodiment 432. The method of any one of embodiments 421-431, wherein cyclophosphamide is administered to the subject intravenously.
Embodiment 433. The method of any one of embodiments 408-432, wherein at least one regimen of fludarabine and at least one regimen of cyclophosphamide is administered to the subject.
Embodiment 434. The method of embodiment 433, wherein the at least one regimen of fludarabine is administered to the subject prior to the administration of the at least one regimen of cyclophosphamide to the subject.
Embodiment 435. The method of embodiment 433 or embodiment 434, wherein the at least one regimen of fludarabine and the at least one regimen of cyclophosphamide are administered to the subject prior to the administration of the dose of engineered hypoimmunogenic islets to the subject.
Embodiment 436. The method of any one of embodiments 433-435, wherein:
   i) a regimen of about 30 mg/m² of fludarabine and a regimen of about 500 mg/m² of cyclophosphamide are administered to the subject each day for 3 consecutive days about 2 days to about 7 days prior to the administration of the dose of engineered hypoimmunogenic islets to the subject;
   ii) a regimen of about 30 mg/m² of fludarabine and a regimen of about 500 mg/m² of cyclophosphamide are administered to the subject each day for 2 consecutive days about 2 days to about 14 days prior to the administration of the dose of engineered hypoimmunogenic islets to the subject; or,
   iii) a regimen of about 30 mg/m² of fludarabine and a regimen of about 500 mg/m² of cyclophosphamide are administered to the subject on day 5, day 4, and day 3 prior to the administration of the dose of engineered hypoimmunogenic islets to the subject.
Embodiment 437. The method of embodiment 436, wherein the fludarabine regimen and/or the cyclophosphamide regimen is administered at a lower dose.
Embodiment 438. The method of any one of embodiments 1-437, further comprising tapering the administration of the one or more immunosuppression agents.
Embodiment 439. The method of embodiment 438, wherein the tapering comprises gradually reducing the amount of the one or more immunosuppression agents that are administered to the subject.
Embodiment 440. The method of embodiment 438 or 439, wherein the tapering is completed when the subject is not administered at least one of the one or more immunosuppression agents.
Embodiment 441. The method of embodiment 440, wherein the tapering is completed when the subject is not administered any immunosuppression agents.
Embodiment 442. The method of any of embodiments 26-441, wherein the one or more molecules that regulate cell surface protein expression of the one or more MHC class I molecules are B2M.
Embodiment 443. The method of any of embodiments 26-442, wherein the modifications comprise a modification that regulates cell surface protein expression of the one or more MHC class I molecules and the modification inactivates or disrupts one or more alleles of B2M.
Embodiment 444. The method of any of embodiments 26-443, wherein the modification that inactivates or disrupts one or more alleles of B2M reduces mRNA expression of the B2M gene.
Embodiment 445. The method of any of embodiments 26-444, wherein the modification that inactivates or disrupts one or more alleles of B2M reduces protein expression of B2M.
Embodiment 446. The method of any of embodiments 26-445, wherein the modification that inactivates or disrupts one or more alleles of B2M comprises:
inactivation or disruption of one allele of the B2M gene;
inactivation or disruption of both alleles of the B2M gene; or inactivation or disruption of all B2M coding alleles in the cell.
Embodiment 447. The method of any of embodiments 26-446, wherein the inactivation or disruption comprises an indel in the B2M gene.
Embodiment 448. The method of any of embodiments 26-447, wherein the inactivation or disruption comprises a frameshift mutation or a deletion of a contiguous stretch of genomic DNA of the B2M gene.
Embodiment 449. The method of any of embodiments 1-448, wherein the modification is a modification that regulates expression of the one or more MHC class II molecules, and the modification inactivates or disrupts one or more alleles of CIITA.
Embodiment 450. The method of embodiment 449, wherein the modification that inactivates or disrupts one or more alleles of CIITA reduces protein expression of CIITA.
Embodiment 451. The method of embodiment 448 or embodiment 449, wherein the modification that inactivates or disrupts one or more alleles of CIITA comprises:
inactivation or disruption of one allele of the CIITA gene;
inactivation or disruption of both alleles of the CIITA gene; or inactivation or disruption of all CIITA coding alleles in the cell.
Embodiment 452. The method of any of embodiments 449-451, wherein the inactivation or disruption comprises an indel in the CIITA gene.
Embodiment 453. The method of any of embodiments 449-451, wherein the inactivation or disruption is a frameshift mutation or a deletion of a contiguous stretch of genomic DNA of the CIITA gene.
Embodiment 454. The method of any of embodiments 1-453, wherein expression of HLA-A, HLA-B, HLA-C, HLA-DP, HLA-DQ, and HLA-DR are reduced in the engineered hypoimmunogenic islets.
Embodiment 455. The method of any of embodiments 1-454, wherein the one or more tolerogenic factors is selected from the group consisting of CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, and MANF.
Embodiment 456. The method of any of embodiments 1-455, wherein at least one of the one or more tolerogenic factors is CD47.
Embodiment 457. The method of any of embodiments 1-456, wherein the one or more tolerogenic factors is CD47, optionally wherein the CD47 is an engineered CD47 protein, more optionally wherein the engineered CD47 protein comprises (a) one or more extracellular domains; and (b) one or more membrane tethers; wherein the one or more extracellular domains comprise a signal-regulatory protein alpha (SIRPα) interaction motif, and wherein the engineered protein does not comprise one or more full-length CD47 intracellular domains, more optionally wherein the SIRPα interaction motif is or comprises a CD47 extracellular domain or a portion thereof.
Embodiment 458. The method of any of embodiments 1-457, wherein the modification that increases expression of the one or more tolerogenic factors comprises an exogenous polynucleotide encoding the one or more tolerogenic factors.
Embodiment 459. The method of embodiment 458, wherein the exogenous polynucleotide encoding the one or more tolerogenic factors is integrated into the genome of the engineered hypoimmunogenic islets.
Embodiment 460. The method of any of embodiments 26-459, wherein the one or more tolerogenic factors comprises CD47 and the engineered hypoimmunogenic islets expresses CD47 at a first level that is greater than at or about 5-fold over a second level expressed by the control or wild-type islet cell.
Embodiment 461. The method of embodiment 460, wherein CD47 is expressed at a first level that is greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by the control or wild-type islet cell.
Embodiment 462. The method of any of embodiments 1-461, wherein the one or more tolerogenic factors comprises CD47 and CD47 is expressed by the engineered hypoimmunogenic islets at greater than at or about 20,000 molecules per cell.
Embodiment 463. The method of embodiment 462, wherein CD47 is expressed by the engineered hypoimmunogenic islets at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.
Embodiment 464. The method of any of embodiments 1-463, wherein the engineered hypoimmunogenic islets has the phenotype B2M^{indeI/indel}; CIITA^{indeI/indel}; CD47tg.
Embodiment 465. The method of any of embodiments 26-464, wherein among the dose of engineered hypoimmunogenic islets, at least 85% of the cells have the modifications.
Embodiment 466. The method of embodiment 465, wherein at least 90%, at least 92%, at least 95% or at least 98% of the cells have the modifications.
Embodiment 467. The method of any of embodiments 26-464, wherein among the dose of engineered hypoimmunogenic islets, at least 85% of the cells have the phenotype has the phenotype B2M^{indeI/indel}; CIITA^{indeI/indel}; CD47tg.
Embodiment 468. The method of embodiment 467, wherein at least 90%, at least 92%, at least 95% or at least 98% of the cells have the phenotype.
Embodiment 469. The method of any of embodiments 1-468, wherein the engineered hypoimmunogenic islets exhibits one or more functions of a wild-type or control beta islet cell, optionally
wherein the one or more functions is selected from the group consisting of in vitro glucose-stimulated insulin secretion (GSIS), glucose metabolism, maintaining fasting blood glucose levels, secreting insulin in response to glucose injections in vivo, and clearing glucose after a glucose injection in vivo.
Embodiment 470. The method of any of embodiments 1-469, wherein the engineered hypoimmunogenic islets is capable of glucose-stimulated insulin secretion (GSIS), optionally wherein the insulin secretion is in a perfusion GSIS assay.
Embodiment 471. The method of embodiment 470, wherein the GSIS is dynamic GSIS comprising first and second phase dynamic insulin secretion.
Embodiment 472. The method of embodiment 470, wherein the GSIS is static GSIS, optionally
wherein the static incubation index is greater than at or about 1, greater than at or about 2, greater than at or about 5, greater than at or about 10 or greater than at or about 20.
Embodiment 473. The method of any of embodiments 1-472, wherein the level of insulin secretion by the engineered hypoimmunogenic islets is at least 20% of that observed for primary islets, optionally cadaveric islets.
Embodiment 474. The method of embodiment 473, wherein the level of insulin secretion by the engineered hypoimmunogenic islets is at least 25%, at least 30%, at least 40%, at least 50%, at least 60%, at least 70% of that observed for primary islets, optionally cadaveric islets.
Embodiment 475. The method of any of embodiments 1-474, wherein the total insulin content of the engineered hypoimmunogenic islets is greater than at or about 500 μIU Insulin per 5000 cells, greater than at or about 1000 μIU Insulin per 5000 cells, greater than at or about 2000 μIU Insulin per 5000 cells, greater than at or about 3000 μIU Insulin per 5000 cells or greater than at or about 4000 μIU Insulin per 5000 cells.
Embodiment 476. The method of any of embodiments 1-475, wherein the proinsulin to insulin ratio of the modified SC-beta cell is between at or about 0.02 and at or about 0.1, optionally at or about 0.03, 0.04, 0.05, 0.06, 0.07, 0.08, 0.09 and any value between any of the foregoing.
Embodiment 477. The method of any of embodiments 1-476, wherein the engineered hypoimmunogenic islets exhibits functionality for more than 2 weeks following administration into a subject.
Embodiment 478. The method of any of embodiments 1-477, wherein the engineered hypoimmunogenic islets exhibits functionality for more than 3 weeks, for more than 4 weeks, for more than 8 weeks, for more than 3 months, for more than 6 months or for more than 12 months following administration into a subject.
Embodiment 479. The method of embodiment 477 or embodiment 478, wherein the functionality is selected from the group consisting of maintaining fasting blood glucose levels, secreting insulin in response to glucose injections in vivo, and clearing glucose after a glucose injection in vivo.
Embodiment 480. The method of any of embodiments 1-479, wherein the dose is from about 1×10⁷ cells to about 3×10⁸ cells.
Embodiment 481. The method of any of embodiments 1-479, wherein the dose is from about 25×10⁶ cells to about 80×10⁷ cells.
Embodiment 482. The method of any of embodiments 1-479, wherein the dose is from about 25×10⁶ cells to about 25×10⁷ cells.
Embodiment 483. The method of any of embodiments 1-479, wherein the dose is from about 80×10⁶ cells to about 80×10⁷ cells.
Embodiment 484. The method of any of embodiments 1-479, wherein the dose is from about 25×10⁶ cells to about 80×10⁶ cells.
Embodiment 485. The method of any of embodiments 1-479, wherein the dose is from about 1.25×10⁵ cells/kg to about 1.2×10⁷ cells/kg.
Embodiment 486. The method of any of embodiments 1-479, wherein the dose is from about 6,500 islet equivalents (IEQ) to about 600,000 IEQ.
Embodiment 487. The method of any of embodiments 1-479, wherein the dose is from about 80 IEQ/kg to about 24,000 IEQ/kg.
Embodiment 488. The method of any of embodiments 1-80 and 442-487, wherein the subject is not administered an immunosuppression regimen.
Embodiment 489. The method of any of embodiments 1-488, wherein the method is characterized by the subject meeting one or more of the following:
a) immune evasion of engineered hypoimmunogenic islets, as evaluated in systemic PBMC and serum;
b) peak c-peptide >0.01 nmol/l in response to a mixed meal tolerance test (MMTT);
c) non-fasting c-peptide concentration >0.01 nmol/l;
d) survival of engineered hypoimmunogenic islets, as evaluated by MRI;
e) decreases in insulin requirement/kg BW;
f) decreases in HbA1c; and
g) reductions in glucose variability, hypoglycemia, and hyperglycemia.
Embodiment 490. The method of embodiment 489, wherein the engineered hypoimmunogenic islets demonstrate immune evasion at 0, 2, 4, 8, 12, 18, 26, and 52 weeks following administration to the subject.
Embodiment 491. The method of embodiment 489, wherein the peak c-peptide is >0.01 nmol/l in response to a MMTT at 4, 8, 12, 18, 26, and 52 weeks following administration of the engineered hypoimmunogenic islets to the subject.
Embodiment 492. The method of embodiment 491, wherein the peak c-peptide is measured by area under the curve (AUC).
Embodiment 493. The method of embodiment 489, wherein the non-fasting c-peptide concentration is >0.01 nmol/l at 1, 2, 3, 4, 6, 8, 10, 12, 14, 16, 18, 20, 22, 24, 26, and 52 weeks following administration of the engineered hypoimmunogenic islets to the subject.
Embodiment 494. The method of embodiment 489, wherein the engineered hypoimmunogenic islets survive within 48 hours following administration to the subject.
Embodiment 495. The method of embodiment 489, wherein the engineered hypoimmunogenic islets survive 2, 4, 6, 8, 12, 26, and 52 weeks following administration to the subject.
Embodiment 496. The method of embodiment 489, wherein the insulin requirement/kg of body weight decreases at 1, 2, 3, 4, 6, 8, 10, 12, 14, 16, 18, 20, 22, 24, 26, and 52 weeks following administration of the engineered hypoimmunogenic islets to the subject.
Embodiment 497. The method of embodiment 489, wherein the HbA1c decreases at 2, 4, 6, 8, 10, 12, 14, 16, 18, 20, 26, and 52 weeks following administration of the engineered hypoimmunogenic islets to the subject.
Embodiment 498. The method of embodiment 489, wherein glucose variability, hypoglycemia, and hyperglycemia are reduced at 4, 8, 12, 18, 26, and 52 weeks following administration of the engineered hypoimmunogenic islets to the subject.
Embodiment 499. The method of any of embodiments 1-498, wherein the subject to be treated is characterized by one or more of the following: diagnosed before the age of 18 years; involved in intensive diabetes management; between ages 18-45; and body weight <80 kg.
Embodiment 500. The method of embodiment 499, wherein intensive diabetes management comprises:
(a) self-monitoring of subcutaneous glucose level by continuous glucose monitoring or by intermittent scanning glucose monitoring no less than a mean of three times per day averaged over each week; and
(b) by the administration of three or more insulin injections per day or insulin pump therapy.
Embodiment 501. The method of any of embodiments 1-498, wherein the subject is not characterized by having the following: any previous organ transplantation; any history of malignancy; use of any investigational agent(s) within 4 weeks of receiving the dose of engineered hypoimmunogenic islets; use of any anti-diabetic medication other than insulin within 4 weeks of receiving the dose of engineered hypoimmunogenic islets; active infections including Tuberculosis, HIV, HBV and HCV; liver function test value for AST, ALT, GGT or ALP exceeding the respective reference interval; serological evidence of infection with HTLVI or HTLVII; pregnancy, nursing, intention for pregnancy; chronic kidney disease grade 3 or worse (GFR <60 ml/min as estimated by creatine measurement); medical history of cardiac disease or symptoms at screening consistent with cardiac disease; HLA immunization, MIC A/B immunization; known autoimmune disease other than type I diabetes (e.g., Hashimoto disease); administration of live attenuated vaccines <6 months before receiving the dose of engineered hypoimmunogenic islets; islet antibodies GADA >2000 IE/mL or IA2A >4000 IE/mL or ZnT8 autoantibodies; untreated proliferative diabetic retinopathy; ongoing psychiatric illness; ongoing substance abuse, drug or alcohol or treatment noncompliance; and known hypersensitivity to ciprofloxacin, gentamicin, or amphotericin.
Embodiment 502. The method of any of embodiments 456-501, wherein the CD47 is an engineered CD47 protein.
Embodiment 503. The method of embodiment 502, wherein the engineered CD47 protein comprises: (a) one or more extracellular domains; and (b) one or more membrane tethers; wherein the one or more extracellular domains comprise a signal-regulatory protein alpha (SIRPα) interaction motif, and
wherein the engineered protein does not comprise one or more full-length CD47 intracellular domains.
Embodiment 502. The method of any of embodiments 456-501, wherein the CD47 is an engineered CD47 protein.
Embodiment 503. The method of embodiment 502, wherein the engineered CD47 protein comprises: (a) one or more extracellular domains; and (b) one or more membrane tethers; wherein the one or more extracellular domains comprise a signal-regulatory protein alpha (SIRPα) interaction motif, and
wherein the engineered protein does not comprise one or more full-length CD47 intracellular domains.
Embodiment 504. The method of embodiment 503, wherein the SIRPα interaction motif is or comprises a CD47 extracellular domain or a portion thereof.
Embodiment 505. The method of embodiment 504, wherein the CD47 extracellular domain is a CD47 immunoglobulin variable (IgV)-like domain.
Embodiment 506. The method of embodiment 503 or 504, wherein the CD47 extracellular domain is a human CD47 extracellular domain.
Embodiment 507. The method of any of embodiment 504-506, wherein the CD47 extracellular domain comprises an amino acid sequence that is at least 80% identical to SEQ ID Nos: 36, 37, 41, and 45.
Embodiment 508. The method of embodiment 503, wherein the SIRPα interaction motif is or comprises a SIRPα antibody or a portion thereof.
Embodiment 509. The method of embodiment 508, wherein the SIRPα antibody or a portion thereof comprises an amino acid sequence that is at least 80% identical to SEQ ID Nos: 64-76.
Embodiment 510. The method of any one of embodiments 503-509, wherein the one or more membrane tethers are or comprise a transmembrane domain.
Embodiment 511. The method of embodiment 510, wherein the transmembrane domain is or comprises a CD3zeta, CD8a, CD16a, CD28, CD32a, CD32c, CD40, CD47, CD64, ICOS, Dectin-1, DNGR1, EGFR, GPCR, MyD88, PDGFR, SLAMF7, TRL1, TLR2, TLR3, TRL4, TLR5, TLR6, TLR7, TLR8, TLR9, or VEGFR transmembrane domain.
Embodiment 512. The method of embodiment 510 or 511, wherein the transmembrane domain is or comprises a CD47 transmembrane domain.
Embodiment 513. The method of any one of embodiments 510-512, wherein the transmembrane domain comprises an amino acid sequence that is at least 80% identical to SEQ ID NO: 38.
Embodiment 514. The method of any one of embodiments 510-512, wherein the transmembrane domain comprises an amino acid sequence that is at least 80% identical to SEQ ID NO: 40.
Embodiment 515. The method of any one of embodiments 510-512, wherein the transmembrane domain comprises an amino acid sequence that is at least 80% identical to SEQ ID NO: 42.
Embodiment 516. The method of any one of embodiments 510-512, wherein the transmembrane domain comprises an amino acid sequence that is at least 80% identical to SEQ ID NO: 44.
Embodiment 517. The method of any one of embodiments 510-512, wherein the transmembrane domain comprises an amino acid sequence that is at least 80% identical to SEQ ID NO: 46.
Embodiment 518. The method of embodiment 510 or 511, wherein the transmembrane domain is or comprises a CD8a transmembrane domain.
Embodiment 519. The method of embodiment 510, 511, or 518, wherein the transmembrane domain comprises an amino acid sequence that is at least 80% identical to SEQ ID NO: 53.
Embodiment 520. The method of embodiment 510 or 511, wherein the transmembrane domain is or comprises a CD28 transmembrane domain.
Embodiment 521. The method of embodiment 510, 511, or 520, wherein the transmembrane domain comprises an amino acid sequence that is at least 80% identical to SEQ ID NO: 55.
Embodiment 522. The method of embodiment 510 or 511, wherein the transmembrane domain is or comprises a PDGFR transmembrane domain.
Embodiment 523. The method of embodiment 510, 511, or 522, wherein the transmembrane domain comprises an amino acid sequence that is at least 80% identical to SEQ ID NO: 57.
Embodiment 524. The method of any one of embodiments 503-523, wherein the one or more membrane tethers are or comprise a glycosylphosphatidylinositol (GPI) anchor.
Embodiment 525. The method of embodiment 524, wherein the GPI anchor is or comprises a DAF/CD55 GPI anchor, a TRAILR3 GPI anchor, or a CD59 GPI anchor.
Embodiment 526. The method of embodiment 524 or 525, wherein the GPI anchor is or comprises a DAF/CD55 GPI anchor.
Embodiment 527. The method of any one of embodiments 524-526, wherein the DAF/CD55 GPI anchor comprises an amino acid sequence that is at least 80% identical to SEQ ID NO: 49.
Embodiment 528. The method of embodiment 524 or 525, wherein the GPI anchor is or comprises a TRAILR3 GPI anchor.
Embodiment 529. The method of any one of embodiments 524, 525, or 528, wherein the TRAILR3 GPI anchor comprises an amino acid sequence that is at least 80% identical to SEQ ID NO: 50.
Embodiment 530. The method of any one of embodiments 524, 525, or 528, wherein the TRAILR3 GPI anchor comprises an amino acid sequence that is at least 80% identical to SEQ ID NO: 51.
Embodiment 531. The method of embodiment 524 or 525, wherein the GPI anchor is or comprises a CD59 GPI anchor.
Embodiment 532. The method of any one of embodiments 524, 525, or 528, wherein the CD59 GPI anchor comprises an amino acid sequence that is at least 80% identical to SEQ ID NO: 52.
Embodiment 533. The method of any one of embodiments 503-532, further comprising an extracellular signal peptide.
Embodiment 534. The method of embodiment 533, wherein the extracellular signal peptide is encoded by a nucleic acid sequence that is at least 80% identical to SEQ ID NO: 35.
Embodiment 535. The method of any one of embodiments 503-534, wherein the one or more extracellular domains further comprise an extracellular hinge domain.
Embodiment 536. The method of embodiment 535, wherein the extracellular hinge domain is or comprises a CD47 hinge, a CD8a hinge, a CD28 hinge, a PDGFR hinge, or an IgG4 hinge.
Embodiment 537. The method of embodiment 535 or 536, wherein the extracellular hinge domain is or comprises a CD47 hinge.
Embodiment 538. The method of any one of embodiments 535-537, wherein the extracellular hinge domain comprises an amino acid sequence that is at least 80% identical to SEQ ID NO: 59.
Embodiment 539. The method of embodiment 535 or 536, wherein the extracellular hinge domain is or comprises a CD8a hinge.
Embodiment 540. The method of embodiment 535, 536, or 539, wherein the extracellular hinge domain comprises an acid sequence that is at least 80% identical to SEQ ID NO: 60.
Embodiment 541. The method of embodiment 535 or 536, wherein the extracellular hinge domain is or comprises a CD28 hinge.
Embodiment 542. The method of embodiment 535, 536, or 541, wherein the extracellular hinge domain comprises an amino acid sequence that is at least 80% identical to SEQ ID NO: 61.
Embodiment 543. The method of embodiment 535 or 536, wherein the extracellular hinge domain is or comprises a PDGFR hinge.
Embodiment 544. The method of embodiment 535, 536, or 543, wherein the extracellular hinge domain comprises an amino acid sequence that is at least 80% identical to SEQ ID NO: 62.
Embodiment 545. The method of embodiment 535 or 536, wherein the extracellular hinge domain is or comprises an IgG4 hinge.
Embodiment 546. The method of embodiment 535, 536, or 545, wherein the extracellular hinge domain comprises an amino acid sequence that is at least 80% identical to SEQ ID NO: 63.
Embodiment 547. The method of any embodiments 503-546, further comprising an intracellular domain.
Embodiment 548. The method of embodiment 547, wherein the intracellular domain is or comprises a CD47 intracellular domain or a portion thereof.
Embodiment 549. The method of embodiment 547 or 548, wherein the intracellular domain comprises an amino acid sequence that is at least 80% identical to SEQ ID NO: 39.
Embodiment 550. The method of embodiment 547 or 548, wherein the intracellular domain comprises an amino acid sequence that is at least 80% identical to SEQ ID NO: 43.
Embodiment 551. The method of embodiment 547 or 548, wherein the intracellular domain comprises an amino acid sequence that is at least 80% identical to SEQ ID NO: 47.
Embodiment 552. The method of embodiment 547 or 548, wherein the intracellular domain comprises an amino acid sequence that is at least 80% identical to SEQ ID NO: 48.
Embodiment 553. The method of embodiment 547, wherein the intracellular domain is or comprises a CD8a intracellular domain or a portion thereof.
Embodiment 554. The method of embodiment 547 or 553, wherein the intracellular domain comprises an amino acid sequence that is at least 80% identical to SEQ ID NO: 54.
Embodiment 555. The method of embodiment 547, wherein the intracellular domain is or comprises a CD28 intracellular domain or a portion thereof.
Embodiment 556. The method construct of embodiment 547 or 548, wherein the intracellular domain comprises an amino acid sequence that is at least 80% identical to SEQ ID NO: 56.
Embodiment 557. The method of embodiment 547, wherein the intracellular domain is or comprises a PDGFR intracellular domain or a portion thereof.
Embodiment 558. The method of embodiment 547 or 557, wherein the intracellular domain comprises an amino acid sequence that is at least 80% identical to SEQ ID NO: 58.
Embodiment 559. The method of any one of embodiments 547-558, wherein the intracellular domain comprises one or more modifications relative to a wild-type CD47 intracellular domain.
Embodiment 560. The method of any one of embodiments 547-559, wherein the intracellular domain comprises one or more deletions relative to a wild-type CD47 intracellular domain
Embodiment 561. The method of any one of embodiments 547-559, wherein the intracellular domain comprises one or more insertions relative to a wild-type CD47 intracellular domain
Embodiment 562. The method of any one of embodiments 547-561, wherein the intracellular domain comprises altered function relative to a wild-type CD47 intracellular domain.
Embodiment 563. The method of any one of embodiments 547-562, wherein the intracellular domain comprises reduced function relative to a wild-type CD47 intracellular domain.
Embodiment 564. The method of any one of embodiments 547-563, wherein the intracellular domain comprises reduced levels of CD47 intracellular signaling relative to a wild-type CD47 intracellular domain.
Embodiment 565. The method of any one of embodiments 547-564, wherein the intracellular domain comprises a non-functional intracellular domain.
Embodiment 566. The method of any one of embodiments 503-565, comprising an amino acid sequence at least 80% identical to a sequence selected from Table 3.
Embodiment 567. The method of any one of embodiments 503-566, comprising an amino acid sequence at least 80% identical to SEQ ID NO: 77.
Embodiment 568. The method of any one of embodiments 503-566, comprising an amino acid sequence at least 80% identical to SEQ ID NO: 78.
Embodiment 569. The method of any one of embodiments 503-566, comprising an amino acid sequence at least 80% identical to SEQ ID NO: 79.
Embodiment 570. The method of any embodiments 503-566, comprising one or more amino acid sequences at least 80% identical to one or more sequences selected from SEQ ID NO: 35-76.
Embodiment 571. The method of any one of embodiments 503-558, comprising one or more amino acid sequences at least 80% identical to SEQ ID NOs: 35, 36, 38, 39, 40, and 41.
Embodiment 572. The method of any one of embodiments 503-558, comprising one or more amino acid sequences at least 80% identical to SEQ ID NOs: 35, 36, 38, 39, and 40.
Embodiment 573. The method of any one of embodiments 503-558, comprising one or more amino acid sequences at least 80% identical to SEQ ID NOs: 35, 36, 38, and 39.
Embodiment 574. The method of any one of embodiments 503-558, comprising one or more amino acid sequences at least 80% identical to SEQ ID NOs: 35, 36, 38, 39, 40, 41, 42, 43, 44, 45, and 46.
Embodiment 575. The method of any one of embodiments 503-558, comprising one or more amino acid sequences at least 80% identical to SEQ ID NOs: 35, 36, 38, 39, 40, 41, 42, 43, 44, and 45.
Embodiment 576. The method of any one of embodiments 503-558, comprising one or more amino acid sequences at least 80% identical to SEQ ID NOs: 35, 36, 38, 39, 40, 41, 42, 43, and 44.
Embodiment 577. The method of any one of embodiments 503-558, comprising one or more amino acid sequences at least 80% identical to SEQ ID NOs: 35, 36, 38, 39, 40, 41, 42, and 43.
Embodiment 578. The method of any one of embodiments 503-558, comprising one or more amino acid sequences at least 80% identical to SEQ ID NOs: 37 and 49.
Embodiment 579. The method of any one of embodiments 503-558, comprising one or more amino acid sequences at least 80% identical to SEQ ID NOs: 37 and 50.
Embodiment 580. The method of any one of embodiments 503-558, comprising one or more amino acid sequences at least 80% identical to SEQ ID NOs: 37 and 51.
Embodiment 581. The method of any one of embodiments 503-558, comprising one or more amino acid sequences at least 80% identical to SEQ ID NOs: 37, 53, and 54.
Embodiment 582. The method of any one of embodiments 503-558, comprising one or more amino acid sequences at least 80% identical to SEQ ID NOs: 37, 55, and 56.
Embodiment 583. The method of any one of embodiments 503-558, comprising one or more amino acid sequences at least 80% identical to SEQ ID NOs: 37, 57, and 58.
Embodiment 584. The method of any one of embodiments 503-558, comprising one or more amino acid sequences at least 80% identical to SEQ ID NOs: 36, 62, 57, and 58.
Embodiment 585. The method of any one of embodiments 503-558, comprising one or more amino acid sequences at least 80% identical to SEQ ID NOs: 36, 53, 55, and 56.
Embodiment 586. The method of any one of embodiments 503-558, comprising one or more amino acid sequences at least 80% identical to SEQ ID NOs: 36, 53, 53, and 54.
Embodiment 587. The method of any one of embodiments 503-558, comprising one or more amino acid sequences at least 80% identical to SEQ ID NOs: 36, 51, 55, and 56.
Embodiment 588. The method of any one of embodiments 503-558, comprising one or more amino acid sequences at least 80% identical to SEQ ID NOs: 36, 51, 53, and 54.
Embodiment 589. The method of any one of embodiments 503-558, comprising one or more amino acid sequences at least 80% identical to SEQ ID NOs: 36, 50, 55, and 56.
Embodiment 590. The method of any one of embodiments 503-558, comprising one or more amino acid sequences at least 80% identical to SEQ ID NOs: 36, 50, 53, and 64.
Embodiment 591. The method of any one of embodiments 503-590, comprising fewer glycosylation modification sites than a wild-type human CD47 protein.
Embodiment 592. The method of embodiment 591, wherein the engineered protein does not comprise an N206 glycosylation site.

### V. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The term “about” as used herein when referring to a measurable value, such as an amount or concentration and the like, is meant to encompass variations of 20%, 10%, 5%, 1%, 0.5%, or even 0.10% of the specified amount. As used herein, including in the appended claims, the singular forms “a,” “or,” and “the” include plural referents unless the context clearly dictates otherwise. For example, “a” or “an” means “at least one” or “one or more.” It is understood that aspects and variations described herein include embodiments “consisting” and/or “consisting essentially of” such aspects and variations.

As used herein, the term “and/or” includes any and all combinations of one or more of the associated listed items.

As used herein, the term “exogenous” with reference to a polypeptide or a polynucleotide is intended to mean that the referenced molecule is introduced into the cell of interest. The exogenous molecule, such as exogenous polynucleotide, can be introduced, for example, by introduction of an exogenous encoding nucleic acid into the genetic material of the cells such as by integration into a chromosome or as non-chromosomal genetic material such as a plasmid or expression vector. Therefore, the term as it is used in reference to expression of an encoding nucleic acid refers to introduction of the encoding nucleic acid in an expressible form into the cell. In some cases, an “exogenous” molecule is a molecule, construct, factor and the like that is not normally present in a cell, but can be introduced into a cell by one or more genetic, biochemical or other methods.

The term “endogenous” refers to a referenced molecule, such as a polynucleotide (e.g. gene), or polypeptide, that is present in a native or unmodified cell. For instance, the term when used in reference to expression of an endogenous gene refers to expression of a gene encoded by an endogenous nucleic acid contained within the cell and not exogenously introduced. A “gene,” includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. The sequence of a gene is typically present at a fixed chromosomal position or locus on a chromosome in the cell.

The term “locus” refers to a fixed position on a chromosome where a particular gene or genetic marker is located. Reference to a “target locus” refers to a particular locus of a desired gene in which it is desired to target a genetic modification, such as a gene edit or integration of an exogenous polynucleotide.

The term “expression” with reference to a gene or “gene expression” refers to the conversion of the information, contained in a gene, into a gene product. A gene product can be the direct transcriptional product of a gene (e.g. mRNA, tRNA, rRNA, antisense RNA, ribozyme, structural RNA or any other type of RNA) or can be a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristoylation, and glycosylation. Hence, reference to expression or gene expression includes protein (or polypeptide) expression or expression of a transcribable product of or a gene such as mRNA. The protein expression may include intracellular expression or surface expression of a protein. Typically, expression of a gene product, such as mRNA or protein, is at a level that is detectable in the cell.

As used herein, a “detectable” expression level, means a level that is detectable by standard techniques known to a skilled artisan, and include for example, differential display, RT (reverse transcriptase)-coupled polymerase chain reaction (PCR), Northern Blot, and/or RNase protection analyses as well as immunoaffinity-based methods for protein detection, such as flow cytometry, ELISA, or western blot. The degree of expression levels need only be large enough to be visualized or measured via standard characterization techniques.

As used herein, the term “differentiation” or “differentiated” refers to a process by which an unspecialized (“uncommitted”) or less specialized cell acquires the features of a specialized cell such as, for example, a pancreatic cell. A differentiated cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. As used herein, the lineage of a cell defines the heredity of the cell, i.e., which cells it came from and to what cells it can give rise. The lineage of a cell places the cell within a hereditary scheme of development and differentiation. A lineage-specific marker refers to a characteristic specifically associated with the phenotype of cells of a lineage of interest and can be used to assess the differentiation of an uncommitted cell to the lineage of interest.

As used herein, the term “increased expression”, “enhanced expression” or “overexpression” means any form of expression that is additional to the expression in an original or source cell that does not contain the modification for modulating a particular gene expression, for instance a wild-type expression level (which can be absence of expression or immeasurable expression as well). Reference herein to “increased expression,” “enhanced expression” or “overexpression” is taken to mean an increase in gene expression and/or, as far as referring to polypeptides, increased polypeptide levels and/or increased polypeptide activity, relative to the level in a cell that does not contain the modification, such as the original source cell prior to the engineering to introduce the modification, such as an unmodified cell or a wild-type cell. The increase in expression, polypeptide levels or polypeptide activity can be at least 5%, 10%, 20%, 30%, 40% or 50%, 60%, 70%, 80%, 85%, 90%, or 100% or even more. In some cases, the increase in expression, polypeptide levels or polypeptide activity can be at least 2-fold, 5-fold, 10-fold, 20-fold, 30-fold, 40-fold, 50-fold, 60-fold, 70-fold, 80-fold, 90-fold, 100-fold, 200-fold or more.

The term “hypoimmunogenic” refers to a cell that is less prone to immune rejection by a subject to which such cells are transplanted. For example, relative to a similar cell that does not contain modifications, such as an unaltered or unmodified wild-type cell, such a hypoimmunogenic cell may be about 2.5%, 5%, 10%, 20%, 30%, 40%, 50%, 60%, 70%, 80%, 90%, 95%, 97.5%, 99% or more less prone to immune rejection by a subject into which such cells are transplanted. Typically, the hypoimmunogenic cells are allogenic to the subject and a hypoimmunogenic cell evades immune rejection in an MHC-mismatched allogeneic recipient. In some embodiments, a hypoimmunogenic cell is protected from T cell-mediated adaptive immune rejection and/or innate immune cell rejection.

Hypoimmunogenicity of a cell can be determined by evaluating the immunogenicity of the cell such as the cell's ability to elicit adaptive and/or innate immune responses. Such immune response can be measured using assays recognized by those skilled in the art.

The term “tolerogenic factor” as used herein include immunosuppressive factors or immune-regulatory factors that modulate or affect the ability of a cell to be recognized by the immune system of a host or recipient subject upon administration, transplantation, or engraftment. Typically a tolerogenic factor is a factor that induces immunological tolerance to an engineered islets so that the engineered islets is not targeted, such as rejected, by the host immune system of a recipient. Hence, a tolerogenic factor may be a hypoimmunity factor. Examples of tolerogenic factors include immune cell inhibitory receptors (e.g. CD47), proteins that engage immune cell inhibitory receptors, checkpoint inhibitors and other molecules that reduce innate or adaptive immune recognition

The terms “decrease,” “reduced,” “reduction,” and “decrease” are all used herein generally to mean a decrease by a statistically significant amount. However, for avoidance of doubt, decrease,” “reduced,” “reduction,” “decrease” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “modification” with reference to a cell refers to any change or alteration of a nucleic acid in the genome of a cell, which may impact gene expression in the cell. For example, a modification includes a genetic modification that results in alterations, additions, and/or deletion of genes or portions of genes or other nucleic acid sequences. A modified cell, such as a genetically modified cell, can also refer to a cell with an added, deleted and/or altered gene or portion of a gene. In some embodiments, the modification is a genetic modification that directly changes the gene or regulatory elements thereof encoding a protein product in a cell, such as by gene editing, mutagenesis or by genetic engineering of an exogenous polynucleotide or transgene. Genetic modifications include, for example, both transient knock-in or knock-down mechanisms, and mechanisms that result in permanent knock-in, knock-down, or knock-out of target genes or portions of genes or nucleic acid sequences Genetic modifications include, for example, both transient knock-in and mechanisms that result in permanent knock-in of nucleic acids sequences Genetic modifications also include, for example, reduced or increased transcription, reduced or increased mRNA stability, reduced or increased translation, and reduced or increased protein stability.

As used herein, “indel” refers to a mutation resulting from an insertion, deletion, or a combination thereof, of nucleotide bases in the genome. Thus, an indel typically inserts or deletes nucleotides from a sequence. As will be appreciated by those skilled in the art, an indel in a coding region of a genomic sequence will result in a frameshift mutation, unless the length of the indel is a multiple of three. A CRISPR/Cas system of the present disclosure can be used to induce an indel of any length in a target polynucleotide sequence.

In some embodiments, the alteration is a point mutation. As used herein, “point mutation” refers to a substitution that replaces one of the nucleotides. A CRISPR/Cas system of the present disclosure can be used to induce an indel of any length or a point mutation in a target polynucleotide sequence.

As used herein, “knock out” includes deleting all or a portion of the target polynucleotide sequence in a way that interferes with the function of the target polynucleotide sequence. For example, a knockout can be achieved by altering a target polynucleotide sequence by inducing an indel in the target polynucleotide sequence in a functional domain of the target polynucleotide sequence (e.g. a DNA binding domain). Those skilled in the art will readily appreciate how to use the CRISPR/Cas systems of the present disclosure to knock out a target polynucleotide sequence or a portion thereof based upon the details described herein.

In some embodiments, the alteration results in a knockout of the target polynucleotide sequence or a portion thereof. Knocking out a target polynucleotide sequence or a portion thereof using a CRISPR/Cas system of the present disclosure can be useful for a variety of applications. For example, knocking out a target polynucleotide sequence in a cell can be performed in vitro for research purposes. For ex vivo purposes, knocking out a target polynucleotide sequence in a cell can be useful for treating or preventing a disorder associated with expression of the target polynucleotide sequence (e.g. by knocking out a mutant allele in a cell ex vivo and introducing those cells comprising the knocked out mutant allele into a subject).

By “knock in” herein is meant a process that adds a genetic function to a host cell. This causes increased levels of the knocked in gene product, e.g. an RNA or encoded protein. As will be appreciated by those in the art, this can be accomplished in several ways, including adding one or more additional copies of the gene to the host cell or altering a regulatory component of the endogenous gene increasing expression of the protein is made. This may be accomplished by modifying the promoter, adding a different promoter, adding an enhancer, or modifying other gene expression sequences.

In some embodiments, an alteration or modification described herein results in reduced expression of a target or selected polynucleotide sequence. In some embodiments, an alteration or modification described herein results in reduced expression of a target or selected polypeptide sequence.

In some embodiments, an alteration or modification described herein results in increased expression of a target or selected polynucleotide sequence. In some embodiments, an alteration or modification described herein results in increased expression of a target or selected polypeptide sequence.

“Modulation” of gene expression refers to a change in the expression level of a gene. Modulation of expression can include, but is not limited to, gene activation and gene repression. Modulation may also be complete, i.e. wherein gene expression is totally inactivated or is activated to wild type levels or beyond; or it may be partial, wherein gene expression is partially reduced, or partially activated to some fraction of wild type levels.

The term “operatively linked” or “operably linked” are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

As used herein, “pluripotent stem cells” have the potential to differentiate into any of the three germ layers: endoderm (e.g. the stomach linking, gastrointestinal tract, lungs, etc.), mesoderm (e.g. muscle, bone, blood, urogenital tissue, etc.) or ectoderm (e.g. epidermal tissues and nervous system tissues). The term “pluripotent stem cells,” as used herein, also encompasses “induced pluripotent stem cells”, or “iPSCs”, or a type of pluripotent stem cell derived from a non-pluripotent cell. In some embodiments, a pluripotent stem cell is produced or generated from a cell that is not a pluripotent cell. In other words, pluripotent stem cells can be direct or indirect progeny of a non-pluripotent cell. Examples of parent cells include somatic cells that have been reprogrammed to induce a pluripotent, undifferentiated phenotype by various means. Such “iPS” or “iPSC” cells can be created by inducing the expression of certain regulatory genes or by the exogenous application of certain proteins. Methods for the induction of iPS cells are known in the art and are further described below. (See, e.g. Zhou et al., Stem Cells 27 (11): 2667-74 (2009); Huangfu et al., Nature Biotechnol. 26 (7): 795 (2008); Woltjen et al., Nature 458 (7239): 766-770 (2009); and Zhou et al., Cell Stem Cell 8:381-384 (2009); each of which is incorporated by reference herein in their entirety.) As used herein, “hiPSCs” are human induced pluripotent stem cells. In some embodiments, “pluripotent stem cells,” as used herein, also encompasses mesenchymal stem cells (MSCs), and/or embryonic stem cells (ESCs).

The terms “polypeptide” and “protein,” as used herein, may be used interchangeably to refer to a series of amino acid residues joined by peptide bonds (i.e. a polymer of amino acid residues), and are not limited to a minimum length. Such polymers may contain natural or non-natural amino acid residues, or combinations thereof, and include, but are not limited to, peptides, polypeptides, oligopeptides, dimers, trimers, and multimers of amino acid residues. Thus, a protein or polypeptide includes include those with modified amino acids (e.g. phosphorylated, glycated, glycosylated, etc.) and amino acid analogs. Full-length polypeptides or proteins, and fragments thereof, are encompassed by this definition. The terms also include modified species thereof, e.g. post-translational modifications of one or more residues, for example, methylation, phosphorylation glycosylation, sialylation, or acetylation.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For instance, where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure. In some embodiments, two opposing and open ended ranges are provided for a feature, and in such description it is envisioned that combinations of those two ranges are provided herein. For example, in some embodiments, it is described that a feature is greater than about 10 units, and it is described (such as in another sentence) that the feature is less than about 20 units, and thus, the range of about 10 units to about 20 units is described herein.

As used herein, “safe harbor locus” refers to a gene locus that allows expression of a transgene or an exogenous gene in a manner that enables the newly inserted genetic elements to function predictably and that also may not cause alterations of the host genome in a manner that poses a risk to the host cell. Exemplary “safe harbor” loci include, but are not limited to, a CCR5 gene, a PPP1R12C (also known as AAVS1) gene, a CLYBL gene, and/or a Rosa gene (e.g. ROSA26).

As used herein, a “target locus” refers to a gene locus that allows expression of a transgene or an exogenous gene. Exemplary “target loci” include, but are not limited to, a CXCR4 gene, an albumin gene, a SHS231 locus, an F3 gene (also known as CD142), a MICA gene, a MICB gene, a LRP1 gene (also known as CD91), a HMGB1 gene, an ABO gene, a RHD gene, a FUT1 gene, and/or a KDM5D gene (also known as HY). The exogenous polynucleotide encoding the exogenous gene can be inserted in the CDS region for B2M, CIITA, CCR5, F3 (i.e., CD142), MICA, MICB, LRP1, HMGB1, ABO, RHD, FUT1, KDM5D (i.e., HY), PDGFRa, OLIG2, and/or GFAP. The exogenous polynucleotide encoding the exogenous gene can be inserted in introns 1 or 2 for PPP1R12C (i.e., AAVS1) or CCR5. The exogenous polynucleotide encoding the exogenous gene can be inserted in exons 1 or 2 or 3 for CCR5. The exogenous polynucleotide encoding the exogenous gene can be inserted in intron 2 for CLYBL. The exogenous polynucleotide encoding the exogenous gene can be inserted in a 500 bp window in Ch-4:58,976,613 (i.e., SHS231). The exogenous polynucleotide encoding the exogenous gene can be insert in any suitable region of the aforementioned safe harbor or target loci that allows for expression of the exogenous gene, including, for example, an intron, an exon or a coding sequence region in a safe harbor or target locus.

As used herein, a “target” can refer to a gene, a portion of a gene, a portion of the genome, or a protein that is subject to regulatable reduced expression by the methods described herein.

As used herein, a “subject” or an “individual,” which are terms that are used interchangeably, is a mammal. In some embodiments, a “mammal” includes humans, non-human primates, domestic and farm animals, and zoo, sports, or pet animals, such as dogs, horses, rabbits, cattle, pigs, hamsters, gerbils, mice, ferrets, rats, cats, monkeys, etc. In some embodiments, the subject or individual is human. In some embodiments, the subject is a patient that is known or suspected of having a disease, disorder or condition.

As used herein, “therapeutically effective amount” refers to an amount sufficient to provide a therapeutic benefit in the treatment and/or management of a disease, disorder, or condition. In some embodiments, a therapeutically effective amount is an amount sufficient to ameliorate, palliate, stabilize, reverse, slow, attenuate or delay the progression of a disease, disorder, or condition, or of a symptom or side effect of the disease, disorder, or condition. In some embodiments, the therapeutically effective amount is also a clinically effective amount. In other embodiments, the therapeutically effective amount is not a clinically effective amount.

As used herein, the term “treating” and “treatment” includes administering to a subject an effective amount of cells described herein so that the subject has a reduction in at least one symptom of the disease or an improvement in the disease, for example, beneficial or desired clinical results. For purposes of this technology, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. In some embodiments, one or more symptoms of a disease or disorder are alleviated by at least 5%, at least 10%, at least 20%, at least 30%, at least 40%, or at least 50% upon treatment of the disease.

For purposes of this technology, beneficial or desired clinical results of disease treatment include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable.

A “vector” or “construct” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors. Methods for the introduction of vectors or constructs into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

### VI. Examples

The following examples are included for illustrative purposes only and are not intended to limit the scope of the invention.

### Example 1: Assessment of B2M^{-/-}; CIITA^{-/-}; CD47tg Non-Human Primate Primary (NHP) Beta Islet Cells in a Transplant Study

Hypoimmune (B2M^{-/-}; CIITA^{-/-}; CD47tg) non-human primate (NHP) primary beta islet cells were produced and transplanted into an allogeneic recipient NHP. Survival and function of the transplanted B2M^{-/-}; CIITA^{-/-}; CD47tg NHP primary islet cells compared to transplanted wild type human primary beta islet cells, were monitored over time.

### A. Methods

Generation of NHP primary beta islet cells and cell engineering. Primary beta islet cells were isolated from NHP pancreas using a standard technique. Such techniques are known in the art. The isolated cells were engineered to knockout B2M and CIITA using standard CRISPR/Cas gene editing techniques, and were transduced with a transgene (tg) encoding exogenous CD47 protein using a lentiviral vector containing a polynucleotide encoding CD47. The hypoimmune islets were sorted by flow cytometry for cells negative for HLA class I/II and for CD47 overexpression.

Transplant study design and administration. 300 NHP islet clusters of about 1,500 cells per cluster were transplanted by i.m. injection into NHPs. Day 0 (d0) was defined as the day of transplantation.

T Cell Enzyme-Linked Immune absorbent SPOT (ELISPOT) Assay. Interferon gamma (IFNg)-secreting CD8(+) T cells in NHP primary beta islet cells were detected by ELISPOT

Flow cytometry. Expression of donor specific antibodies (DSA) in primary beta islet cells was assessed by flow cytometry.

NK cell killing assay. NK cell killing assays were performed substantially as described in examples above.

### B. Results

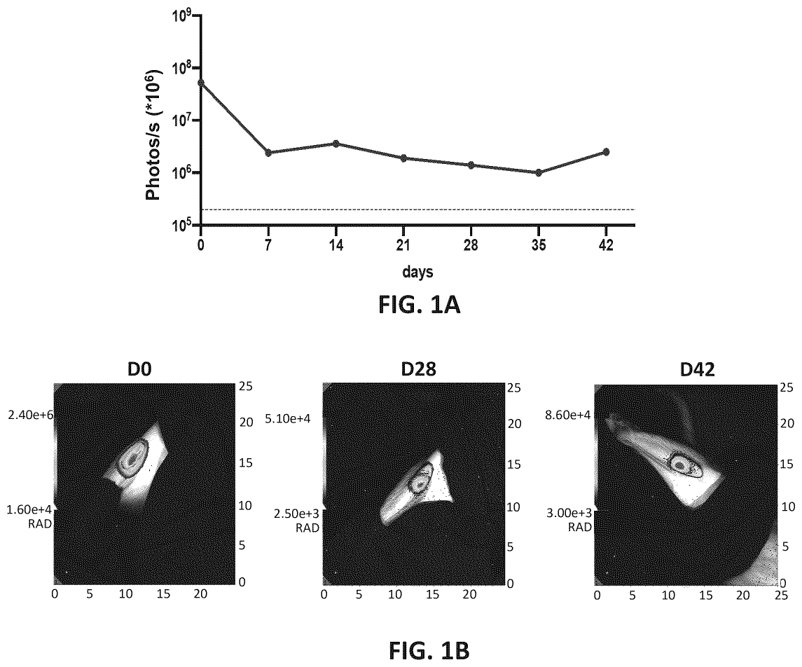

B2M^{-/-}; CIITA^{-/-}; CD47tg NHP islet cells survive after transplant. Quantification of BLI imaging results of NHP B2M^{-/-}; CIITA^{-/-}; CD47tg primary islet cells after i.m. injection is shown in FIG. 1A. Corresponding BLI images of NHP B2M^{-/-}; CIITA^{-/-}; CD47tg primary islet cells after i.m. injection are shown in FIG. 1B. Bioluminescence was initially observed at the i.m. injection site for all groups following administration of the NHP primary islet cells. The number of photons detected from transplanted NHP B2M^{-/-}; CIITA^{-/-}; CD47tg primary islet cells initially slightly decreased post-transplant, but then remained consistent over the course of 42 days post-transplant, indicating survival of the NHP B2M^{-/-}; CIITA^{-/-}; CD47tg primary islet cells (FIG. 1A).

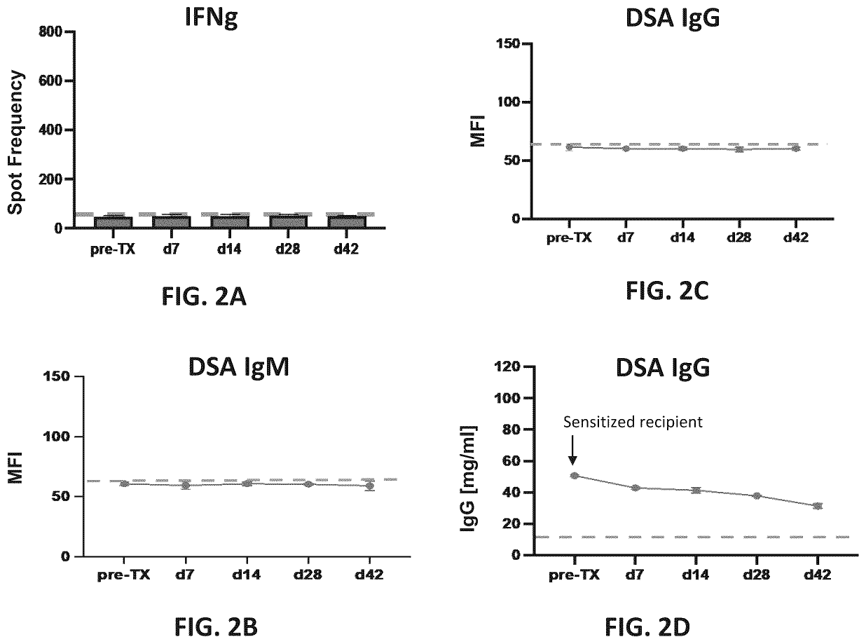

NHP B2M^{-/-}; CIITA^{-/-}; CD47tg primary islet cells immune response after allogeneic transplant. To analyze the immune response to transplanted NHP primary beta islet cells, an ELISPOT assay was used to evaluate the levels of IFNg cytokine secretion by CD8+ T cells. As shown in FIG. 2A, transplanted NHP B2M^{-/-}; CIITA^{-/-}; CD47tg primary islet cells exhibited low levels of IFNg. The levels of DSA IgM and IgG, measured by flow cytometry, were also low in transplanted NHP B2M^{-/-}; CIITA^{-/-}; CD47tg primary islet cells (FIG. 2B and FIG. 2C, respectively). Moreover, in a sensitized NHP recipient with high IgG antibody concentrations prior to transplant with NHP B2M^{-/-}; CIITA^{-/-}; CD47tg primary islet cells, the levels of DSA IgG decreased over the course of 42 days-post transplant (FIG. 2D).

These data indicate that the NHP B2M^{-/-}; CIITA^{-/-}; CD47tg primary islet cells do not induce an immune response to the cells upon transplantation and are protected from antibody-mediated rejection.

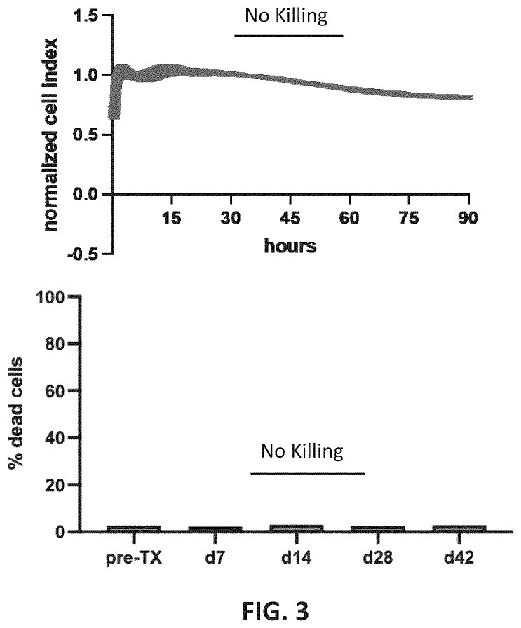

B2M^{-/-}; CIITA^{-/-}; CD47tg NHP primary islet cells evade killing by NK cells. B2M^{-/-}; CIITA^{-/-}; CD47tg NHP primary islet cells did not exhibit NK-mediated cell killing (FIG. 3), indicating that the hypoimmune (B2M^{-/-}; CIITA^{-/-}; CD47tg) NHP primary islet cells are able to effectively evade immune responses by NK cells. These data indicate that transplanted B2M^{-/-}; CIITA^{-/-}; CD47tg NHP primary islet cells are not recognized as foreign.

### Example 2: Assessment of B2M^{-/-}; CIITA^{-/-}; CD47tg Non-Human Primate (NHP) Primary Beta Islet Cells for Diabetes Treatment in a NHP

Hypoimmune (B2M^{-/-}; CIITA^{-/-}; CD47tg) non-human primate (NHP) primary beta islet cells were produced and transplanted into an allogeneic recipient NHP. Specifically, hypoimmune (B2M^{-/-}; CIITA^{-/-}; CD47tg) rhesus macaque primary beta islet cells were produced and transplanted into an allogeneic recipient cynomolgus monkey. Phenotyping, cellular and antibody-mediate responses, fasting glucose levels, glucose tolerance, and C-peptide in serum were monitored over time.

### A. Methods

Animals. A one-year-old male cynomolgus macaque served as islet transplant recipient. The cynomolgus monkey recipient was selected as STZ-inducible diabetes mellitus can most reliably be induced in this strain.

Four male rhesus macaques (5-10 kg) were used as islet donors. The rhesus macaque donor was selected not only because it is a different strain in the Macaca genus and thus considered allogeneic, but also because it has major differences in its MHC repertoire. Specifically, in macaques, evolutionary orthologues of HLA have undergone several rounds of duplication, which lead to a complex MHC system with significant copy number variation (Otting et al., Immunogenetics (2007) 59:367-375). This strategy does not utilize allogeneic transplants within pedigreed colonies that are often Mafa-A or Mafa-B haploidentical or MHC haploidentical due to the limited MHC repertoire (Wiseman et al., Nat Med (2009) 15:1322-1326) and that generate more favorable transplant results (Lei et al., Sci Adv (2022) 8:eabm9881). In addition, other typical tolerance-inducing strategies require careful MHC class II matching (Singh et al., Nat Commun (2019) 10:3495).

All rhesus donors and the cynomolgus recipient were blood type B to avoid blood group incompatibility, as it is standard in clinical allotransplantation.

Pancreas procurement and islet isolation. Briefly, each of four rhesus macaques were exsanguinated. The pancreatic tail was identified and separated from the spleen. Rotating the greater curvature of the stomach anteriorly, the pylorus and duodenum were identified. The duodenum was cut just past the pyloric valve and then distally when it was clear of the pancreatic tissue. The pancreas was then freed from any remaining tissues. The harvested pancreas was preserved with cold University of Wisconsin (UW) solution for islet cell isolation.

Pancreata were removed from UW solution and an 18 gauge plastic IV catheter was inserted into the pancreatic duct. The pancreas was inflated with 50-80 ml of RT Media (RPMI 1640 medium (Thermo Fisher), 10% FBS, 100 U pen/strep) containing 0.6 mg collagenase P (Sigma Aldrich). After inflation, fat and connective tissue was removed from the tissue and the tissue was divided into 12 equal portions. Each portion was placed in a 50 ml conical tube containing 11 ml of RT medium with 0.06 mg collagenase P and incubated in a 37° C. water bath for 27 minutes with agitation every 10 minutes. After digestion, collagenase was poured off, and 10 ml of RT media was added and shaken for 1 minute to disrupt remaining connective tissue. Undigested material was removed by straining through a 500 micron filter and islets were washed 3 times with 50 ml of RT media, followed by a 10 minute Bovine DNaseI (Sigma-Aldrich, 0.8 U/ml) incubation in a 37° C. water bath. Islets were cultured overnight at 37° C./5% CO₂ in RT media with Bovine DNaseI (Sigma-Aldrich, 0.4 U/ml).

The cynomolgus monkey recipient had an estimated pancreas mass of 5 g with about 50 million beta cells (Ferrannini et al., Cell Metab (2010) 11:349-352; Amato et al., Toxicol Pathol (2022) 50:574-590). The yield of rhesus macaque B2M^{-/-}; CIITA^{-/-}; CD47tg islet cells from each of the 4 pancreata was approximately 11% and the overall transplanted number of pooled rhesus macaque B2M^{-/-}; CIITA^{-/-}; CD47tg islet cells was 30 million, representing approximately 60% of the original endogenous beta cell mass.

Generation of NHP primary beta islet cells and cell engineering. Primary beta islet cells were isolated from four rhesus macaque pancreata using the technique described above. The isolated cells were engineered to knockout B2M and CIITA using standard CRISPR/Cas gene editing techniques, and were transduced with a transgene (tg) encoding exogenous CD47 protein using a lentiviral vector containing a polynucleotide encoding CD47. Particularly, primary rhesus macaque cadaveric islets were isolated using a standard technique known in the art. The CRISPR/Cas9 technology was used for the disruption of the B2M and CIITA genes. Islet clusters were dissociated in single cells using AccuMax (StemCell Technologies) for 10 min at 37° C. The following gRNA sequences were used for the Macaca mulatta B2M gene 5′-CGUGAGUAAACCUGAAUCUU-3′ (SEQ ID NO: 29) and Macaca mulatta CIITA gene 5′-GAUAUUGGCAUAAGCCUCCC-3′ (SEQ ID NO: 30). Lonza P3 Primary Cell 4D-Nucleofector™ X Kit (cat.no V4XP-3032, Lonza) was used for the transfection of the islet cells. Briefly, cells were transduced at a final concentration of 50 million cells per ml in P3 buffer. The cell suspensions (20 pl) were combined with 13 pg Cas9 enzyme and 5 pM sgRNA. Lonza's 4D-Nucleofector was used for the electroporation with the preset program CA-137. Islet cells were transferred to U-bottom 96-well plates at a density of 50,000 cells per well in PIM(S) media (Prodo) and rested for 1 hour at 37° C. and 5% CO₂ before setting the plate on the belly dancer orbital shaker (IBI Scientific, Dubuque, IA) for islet re-clustering. Media was changed after 48 hour and islet clusters were incubated on the belly dancer for another 24 h. Islet clusters were dissociated again into single cells using AccuMax for cells sorting using the anti-HLA-A,B,C antibody (clone G46_2.6,BD Biosciences), which cross-reacts with rhesus MHC class I, or IgG1 isotype-matched control antibody (clone MOPC-21,BD Biosciences) and anti-HLA-DR,DP,DQ antibody (clone Tu3a, BD Biosciences), which cross-reacts with rhesus MHC class II or IgG2a isotype-matched control antibody (clone G155-178, BD Biosciences). Double negative cells were sorted in the BD FACS Aria II and replated in U-bottom 96-well plates as described above for islet re-clustering on the belly dancer orbital shaker. After 24 h, islets were dissociated into single cells for rhesus CD47 transduction with a CAG-CD47 lentiviral vector (LVV) (custom order, Thermo Fisher) at a MOI of 5. Spinfection was performed with the presence of 10 μg/ml protamine sulfate at 300 g for 15 min. Cells were replated in U-bottom 96-well plates as described above for islet re-clustering on the belly dancer orbital shaker. After 48 hours, cells were dissociated in single cells using AccuMax and underwent cell sorting for rhesus CD47 with anti-CD47 antibody (clone CC2C6, BD Biosciences), which cross-reacts with rhesus CD47 or IgG1 isotype-matched control antibody (clone MOPC-21, BD Biosciences) on BD FACS Aria II. Islet cells were pooled and replated in low-attachment 6-well plates (3471, Corning) for islet re-clustering on the belly dancer orbital shaker until transplantation.

Flow cytometry. The engineered hypoimmune islets were sorted by flow cytometry for cells negative for HLA class I/II and for CD47 overexpression. Specifically, rhesus macaque islet cells were dissociated into single cells using AccuMax (StemCell Technologies) for 10 min at 37° C. and labeled with APC-conjugated anti-HLA-A,B,C antibody (clone G46_2.6 has shown cross-reactivity with rhesus macaque MHC class I, BD Biosciences). APC-conjugated IgG1 isotype-matched control antibody (clone MOPC-21,BD Biosciences), Alexa-flour647-labeled anti-HLA-DR,DP,DQ antibody (clone Tu39, BD Biosciences), Alexa-flour647-labeled IgG2a isotype-matched control antibody (clone G155-178, BD Biosciences), FITC-conjugated anti-CD47 antibody (clone CC2C6, BioLegend), or FITC-conjugated mouse IgG1k isotype-matched control antibody (MOPC-21, BioLegend). Approximately 11-15% of the isolated pancreatic islet cells were hypoimmune islets and were used for administration as described below.

Quantification ofislet cluster insulin production. One hundred rhesus macaque islet cells were plated in one 6-well with 2 ml of islet media containing 5.8 mM glucose (PIM(S) media, Prodo). Supernatants were collected after 24 hour and ELISA assays for rhesus insulin (MBS701773, MyBiosource) were performed according to the manufacturer's protocol. A microplate reader with an absorbance of optical density (OD) 450 nm (Molecular Devices) was used to measure the insulin level of the standards and study samples. Insulin levels were calculated to plU per million cells.

Islet composition. Rhesus macaque islet cells were dissociated with AccuMax (StemCell Technologies) for 10 min at 37° C. The following antibodies were used to detect islet composition: anti-insulin antibody (clone 2D11-H5, Santa Cruz Biotechnology), anti-glucagon antibody (clone C-11, Santa Cruz Biotechnology) and anti-somatostatin antibody (clone G-10, Santa Cruz Biotechnology).

Islet immunofluorescence. Approximately 20 ml of aggregates was collected into a 1.5 ml Eppendorf tube and spun down. The pellet was resuspended in fixation/permeabilization working solution (cat.no. 00-5523-00, eBioscience) and incubated overnight at 4° C. Cells were washed with permeabilization working buffer (cat.no. 00-5523-00, eBioscience) and stained with 5 ml each of glucagon (cat.no. NBP2-21803AF647, Novus Biologicals), somatostatin (cat.no. NBP2-99309AF350, Novus Biologicals), and insulin (cat.no. 53-9769-82, Life Technologies) for 24 hours at 4° C. Cells were washed with permeabilization working buffer, mounted to slides with Prolong Gold (cat.no. P36930, Fisher Scientific), and allowed to dry overnight. For MHC and rhesus CD47 immunofluorescence, the same amount of islets were collected and washed with stain buffer (DPBS with 0.1% BSA and 5 mM EDTA). Aggregates were stained with 5 ml of PE anti-human CD47 (cat.no. 323108, Biolegend) or APC anti-human HLA-ABC (cat.no. 562006, BD Bioscience) for 45 minutes on ice and washed with stain buffer. Cells were fixed with BD Cytofix on ice for 30 minutes (cat.no. 554655, Fisher Scientific), washed with stain buffer, mounted on slides with Prolong Gold with DAPI (cat.no. P36931, Fisher Scientific), and allowed to dry overnight. All slides were then imaged on Leica Thunder Imaging System.

Histology. The islet injection sites in the left and right quadriceps muscles were fixed in 10% neutral buffered formalin, cut into 2-3 mm pieces, and embedded in paraffin. A pancreas from a healthy cynomolgus monkey and the pancreas from the cynomolgus monkey were processed similarly. Blocks were sectioned at 4 μm and stained with hematoxylin and eosin (H&E) and anti-Islet 1 antibody (Abcam, ab178400). Sections that were positive for Islet 1 staining (pancreas) were stained for insulin (Abcam, abl81547), glucagon (Abcam, 92517) and somatostatin (Abcam, ab111912). The healthy pancreas with no STZ treatment was used as a positive control and to compare beta cell mass following STZ treatment. Immunohistochemical staining was performed on the Leica Bond Rxm using the Leica Bond Refine kit (Leica Biosystems—DS9800) for DAB chromogenic staining. Blocking of non-specific binding was done with 1× Animal Free Blocker (SP-5030-250) in 1% NGS (Cell Signaling—5425S) diluted in TBST (Thermo Fisher—J77500.K2) which also served as the antibody diluent. Slides were dehydrated, cleared, coverslipped, and scanned using a Leica Aperio 200.

Transplant study design and administration. The cynomolgus monkey was fasted for 12 hours prior to receiving a single (100 mg/kg) intravenous injection of STZ. The STZ (cat.no S0130, Sigma) was first dissolved in citrate buffer (100 mg STZ and 22 mg citric acid per ml saline) and then diluted to the final concentration in 12 ml cold saline. On Day 0 (d0), a cynomolgus monkey received a single i.v. injection of streptozotocin (STZ) at a concentration of 100 mg/kg. STZ was administered via the saphenous vein. On d3, the cynomolgus monkey received insulin (Lantus Sanofi, Paris, France) at a concentration of 2 units/day to maintain blood glucose within normal range (30-80 mg/dl). On d78, 50 million engineered hypoimmune islet cells were transplanted by i.m. injection into the cynomolgus monkey (administered as clusters by separate injections of 25 million cells each in different limbs). The cynomolgus monkey was monitored for maintenance of fasting blood glucose levels using a Glucotrend monitor (Roche Instruments, Basel, Switzerland). Blood glucose levels were monitored twice daily. On d85, the cynomolgus monkey began receiving a reduced concentration of insulin at 1 unit/day. On d88, the concentration of insulin was reduced again to 0.5 unit/day. On d89, insulin administration ceased.

An anti-CD47 antibody was given (magrolimab 5F9 sequence, Creative Biolabs) at a dose of 50 mg (diluted in PBS, along with Depo-Medrol 40 mg/kg, Pfizer, New York, NY) and dosed alternately intramuscularly or intraperitoneally for a total of 9 days.

Anesthesia was performed using ketamine (10-20 mg/kg), tiletamine and zolazepam (5-8 mg/kg), and isoflurane (1-4%). Fifty million hypoimmune (B2M^{-/-}; CIITA^{-/-}; CD47tg) rhesus macaque primary beta islet cells were transplanted into both quadricep muscles of the cynomolgus monkey. Islet clusters were resuspended in 100 pl RPMI-1640 media (Thermo Fisher) including pro-survival cocktail (200 M ZVAD and 100 nM BcL-xL (both Millipore), 200 ng/ml IGF-1 (PeproTech), 100 M pinacidil, and 200 nM cyclosporine A (both Sigma-Aldrich)) and loaded into 1 ml syringes without dead volume with a 23 G needle. A 2 cm skin incision was made over the middle anterior side of the quadricep muscles, and the rhesus macaque islet cells were injected in a string pattern parallel to the muscle fibers. The incisions were closed with 5-0 absorbable suture using interrupted subcuticular stitches. Buprenorphine SR (0.2 mg/kg) and meloxicam SR (0.3 mg/kg) were given subcutaneously for analgesia and animals were returned to home housing.

Injections into the muscle circumvent early islet loss through an instant blood-mediated inflammatory reaction (IBMIR) that is known to occur after portal vein injections (Bennet et al., Diabetes (1999) 48:1907-1914). The muscle is well vascularized and islet transplantations into striated muscle have been successful clinically (Christoffersson et al, Diabetes (2010) 59:2569-2578; Rafael et al., Am J Transplant (2008) 8:458-462).

Fasting Blood Glucose Assay. Blood was collected daily from the femoral vein of cynomolgus monkey using a 22G needle, vacutainer sheath, and collection tube, in the morning and in the afternoon. Following venipuncture, manual compression of the vein was maintained until hemostasis was achieved. Blood collection was based on weight of the animals not exceeding AGI maximum bleeds. Fasting glucose was measured in mg/dL. According the American Diabetes Association, plasma glucose level criteria for normal humans was identified as <4.4 mmol/L, impaired fasting plasma glucose as 4.4-6.99 mmol/L, and diabetic as >6.99 mmol/L (Yue et al., Lipids in Health and Disease (2016) 15:111). Since 1 mg/dL equals approximately 0.55 mmol/L, the blood glucose data from the cynomolgus monkey was multiplied by 0.055 to characterize the cynomolgus monkey fasting blood glucose levels to those of a human who is diabetic, has impaired fasting glucose, is normal or is hypoglycemic.

C-peptide Assay. Blood was collected from the cynomolgus monkey before STZ administration, and again on days 50 (d50), 85 (d85), and 92 (d92) of the study. Blood was further processed to purify serum using standard techniques and C-peptide measured. Specifically, the cynomolgus c-peptide ELISA kit (Novus Biologicals, Littleton, CO) was used to measure cynomolgus c-peptide in serum. Samples were diluted and pipetted according to manufactures instructions. Briefly, standards and samples were added to pre-coated 96-well ELISA plates and incubated for 1 hour. After the removal of unbound proteins by washing, anti-c-peptide antibodies conjugated with horseradish peroxidase, were added. These enzyme-labeled antibodies form complexes with the previously bound c-peptide. The enzyme bound to the immunosorbent is assayed by the addition of a chromogenic substrate, tetramethylbenzidine. Samples were analyzed in a microplate reader (Perkin Elmer, Waltham, MA).

Glucose Tolerance Test. Glucose tolerance was measured in the cynomolgus monkey pre-STZ administration and again on d50 and day 103 (d103) using standard techniques.

Elispot. For uni-directional Elispot assays, recipient PBMCs were isolated from the cynomolgus recipient at different time points and CD3+ T cells were sorted (BD Aria Fusion). Rhesus macaque islet cells were treated with mitomycin (50 g/ml for 30 min, Sigma) and used as stimulator cells. A total of 1×10⁷ stimulator cells were incubated with 5×10⁷ recipient responder T cells for 36 hour and IFN-γ spot frequencies were enumerated using an Elispot plate reader (AID, Strassberg, Germany).

Total IgMand IgG ELISA. The total rhesus IgM ELISA kit (MyBioSource, San Diego, CA) and total rhesus IgG ELISA kit (Molecular-Innovations, Novi, MI) were used to measure total IgM or IgG in the cynomolgus monkey serum. Samples were diluted and pipetted according to manufacturer's instructions. Briefly, standards and samples were added to pre-coated 96-well ELISA plates and incubated for 1 hour. After the removal of unbound proteins by washing, anti-IgM or anti-IgG antibodies conjugated with horseradish peroxidase, were added. These enzyme-labeled antibodies form complexes with the previously bound IgM or IgG. The enzyme bound to the immunosorbent is assayed by the addition of a chromogenic substrate, tetramethylbenzidine. Samples were analyzed in a microplate reader (Perkin Elmer).

Donor Specific Antibody (DSA). Sera from recipient animals were de-complemented by heating to 56° C. for 30 min. Equal amounts of sera and cell suspensions (5×10⁶ per ml) were incubated for 45 min at 4° C. Cells were labeled with FITC-conjugated goat anti-rhesus IgM (BioLegend) or FITC-conjugated goat anti-rhesus IgG (Southern Biotech) and mean fluorescence intensity (MFI) was analyzed by flow cytometry (Attune, ThermoFisher).

Cynomolgus macaque NK cell isolation. Cynomolgus PBMCs were sorted on the FACSAria Fusion using FITC-conjugated anti-CD8 (ab28010, 1:5, Abcam) and PE-conjugated anti-NKG2A (130-114-092, Miltenyi) antibodies to select a CD8+NKG2A+NK cell population.

Macrophage differentiation from PBMCs. PBMCs were isolated by Ficoll separation from fresh blood and were resuspend in RPMI-1640 with 10% heat-inactivated fetal calf sera (FCS hi) and 1% Pen/Strep (all Gibco). Cells were plated in 24-well plates at a concentration of 1×10⁶ cells per ml and 10 ng/ml rhesus M-CSF (Biorbyt, Cambridge, Great Britain) was added. Media was changed every other day until day 6. Macrophages were stimulated from day 6 with 1 g/ml rhesus IL-2 (MyBiosource) for 24 hours before the cells were used.

In vivo Ablation of transplanted rhesus macaque primary beta islet cells. After Day 190, an anti-CD47 antibody (magrolimab 5F9 sequence, Creative Biolabs) was administered intramuscularly or intraperitoneally to the cynomolgus monkey at a dose of 50 mg (diluted in PBS, along with Depo-Medrol 40 mg/kg, Pfizer, New York, NY) for a total of 9 days.

In vitro killing assay of edited rhesus macaque primary beta islet cells. Rhesus macaque primary beta islet cell killing was tested in vitro. Cynomolgus NK cells and macrophages were differentiated from PBMCs. For NK cell generation, PBMCs were sorted on the FACSAria Fusion using FITC-conjugated anti-CD8 (ab28010, 1:5, Abcam) and PE-conjugated anti-NKG2A (130-114-092, Miltenyi) antibodies to select for CD8+NKG2A+NK cell population. For macrophage generation, PBMCs were isolated by Ficoll separation from fresh blood and were resuspended in RPMI-1640 with 10% heat-inactivated fetal calf sera (FCS hi) and 1% Pen/Strep (all Gibco). Cells were plated in 24-well plates at a concentration of 1×10⁶ cells per ml and 10 ng/ml rhesus M-CSF (Biorbyt, Cambridge, Great Britain) was added. Media was changed every other day until day 6. Macrophages were stimulated from day 6 with 1 g/ml rhesus IL-2 (MyBiosource) for 24 hours before the cells were used.

Cynomolgus monkey PBMC, T cell, NK cell, and macrophage killing assays were performed on specialized 96-well plates coated with collagen and fibronectin (Sigma-Aldrich) and 4×10⁴ target islet cells were plated in 100 l media. After the Cell Index reached 0.7, the effector cells were added at an effector cell to target cell (E:T) ratio of 1:1. NK cells were stimulated with 1 g/ml rhesus IL-2 (Peprotech). anti-CD47 antibody (Magrolimab) was added in a concentration of 100 μg/ml. As killing control, cells were treated with 2% TritonX100 in water. Islet cells were collected 90 hours after adding the effector cells and were analyzed for the percentage of dead cells via flow cytometry (Zombie live/dead stain, Biolegend). Data were standardized and analyzed with the RTCA software (ACEA).

Complement-dependent cytotoxicity (CDC) and antibody-dependent cell-mediated cytotoxicity (ADCC) killing by Xcelligence. CDC and ADCC killing assays were also performed on the XCelligence MP platform. For CDC assays, 100 μl of untreated, complement-containing serum (1:1 mixed with media) was added. For ADCC assays, 50 pl heat-inactivated serum with 4×10⁴ cynomolgus macaque NK cells or macrophages in 50 pl media were added. As a killing control, cells were treated with 2% TritonX100 in water. As a survival control, cells were only incubated with media. Data were standardized and analyzed with the RTCA software (ACEA).

### B. Results

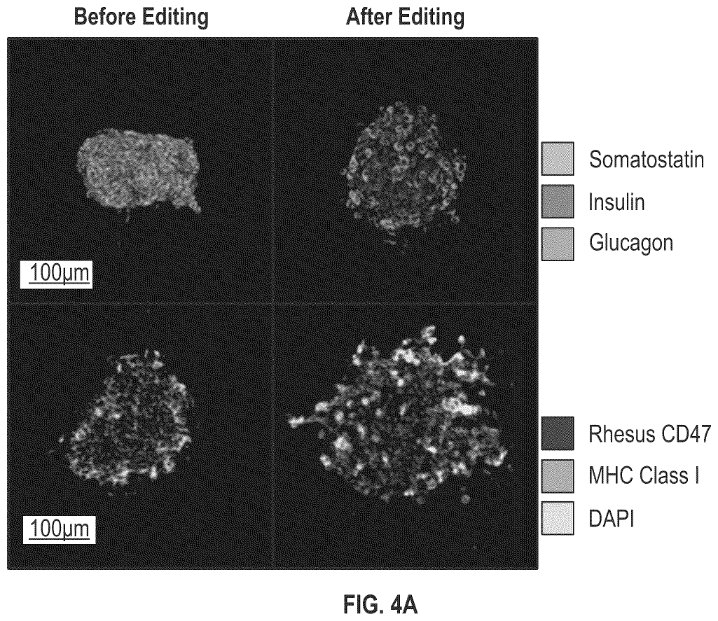

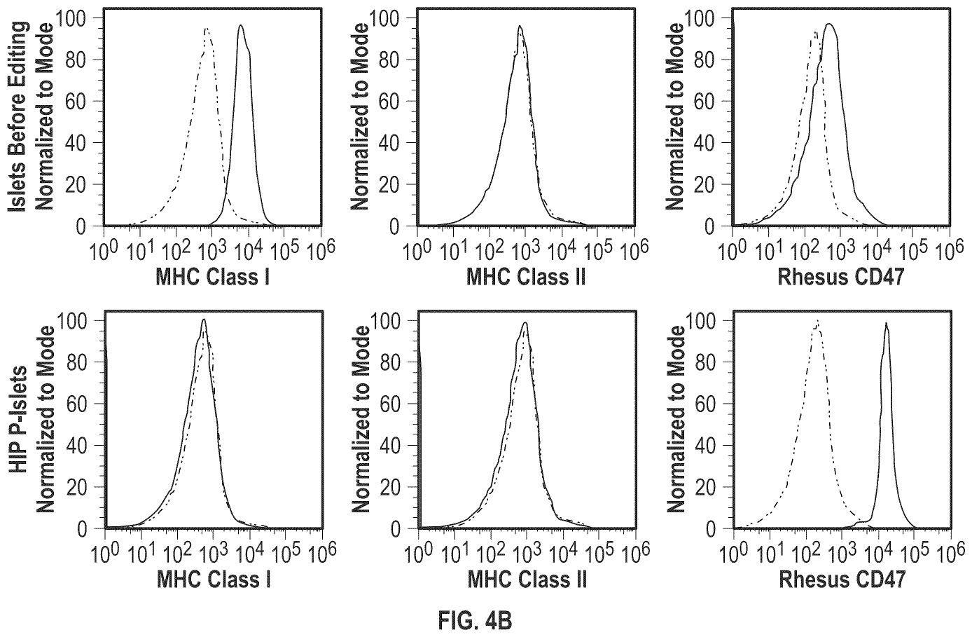

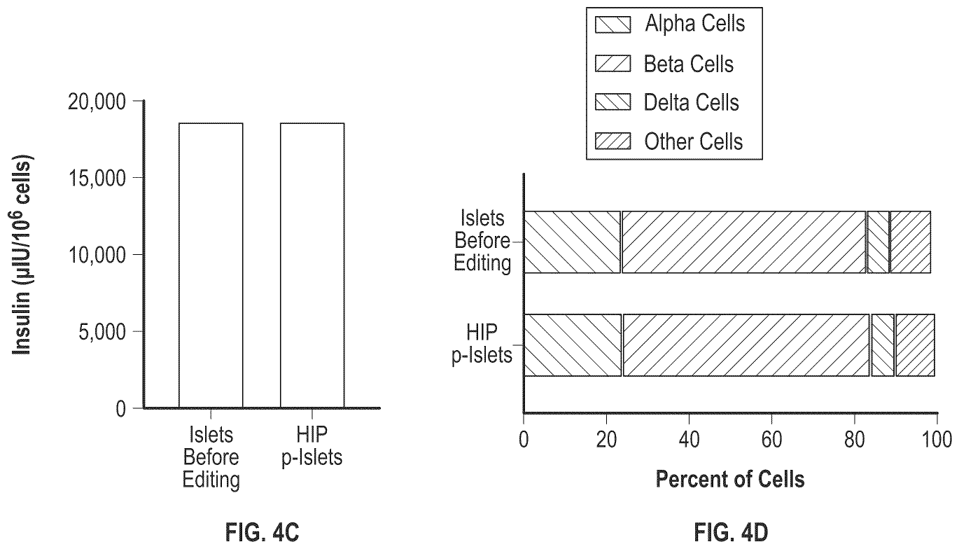

Engineered primary beta cell islets showed morphological and phenotypical similarity to non-engineered primary beta cell islets. Rhesus macaque B2M^{-/-}; CIITA^{-/-}; CD47tg primary islets were morphologically similar to primary rhesus macaque islets (FIG. 4A) and showed the typical B2M^{-/-}; CIITA^{-/-}; CD47tg phenotype (FIG. 4B). The rhesus macaque B2M^{-/-}; CIITA^{-/-}; CD47tg primary islets also secreted insulin in vitro (FIG. 4C) and approximately 60% of cells in the primary islets were beta cells (FIG. 4D).

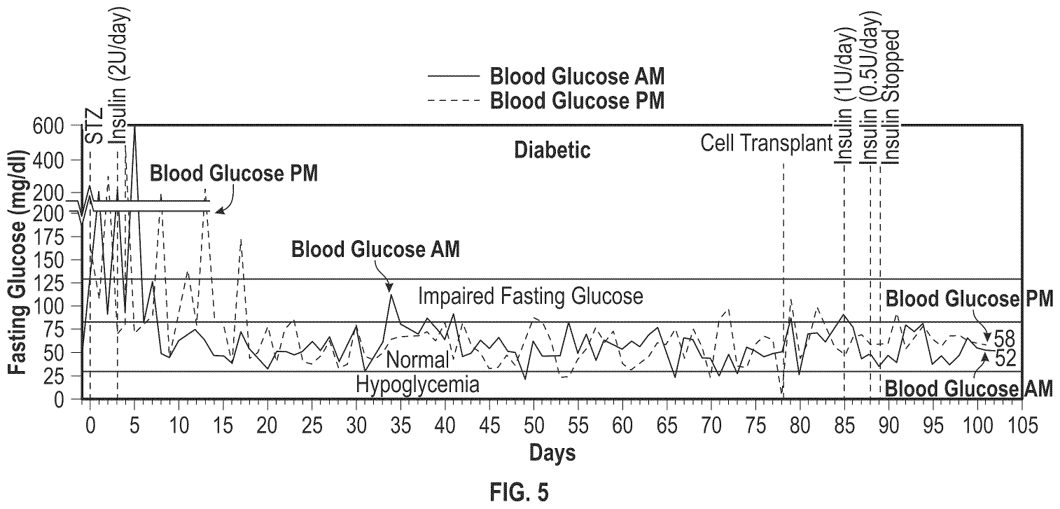

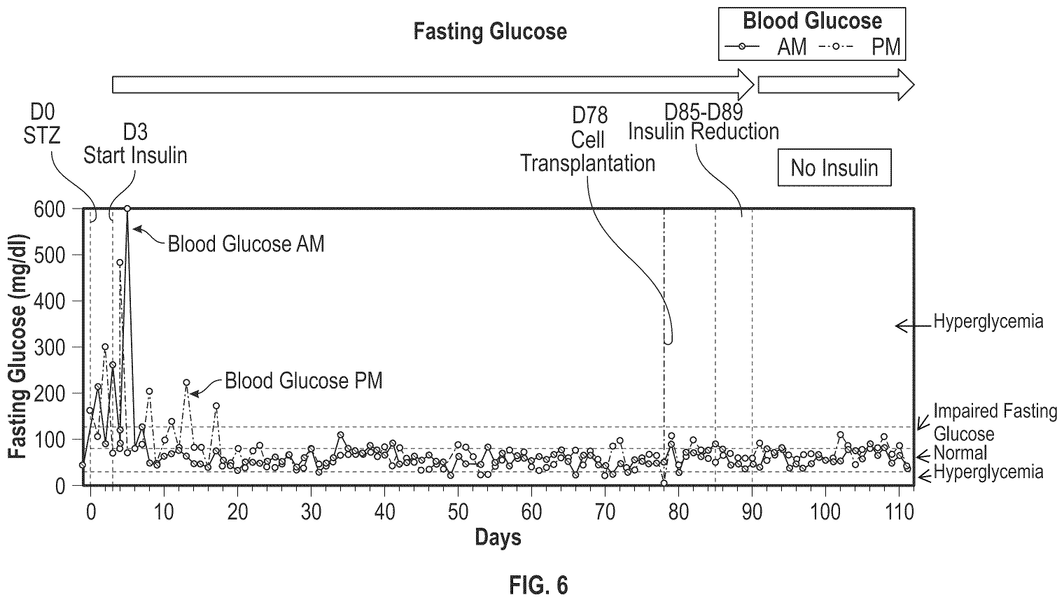

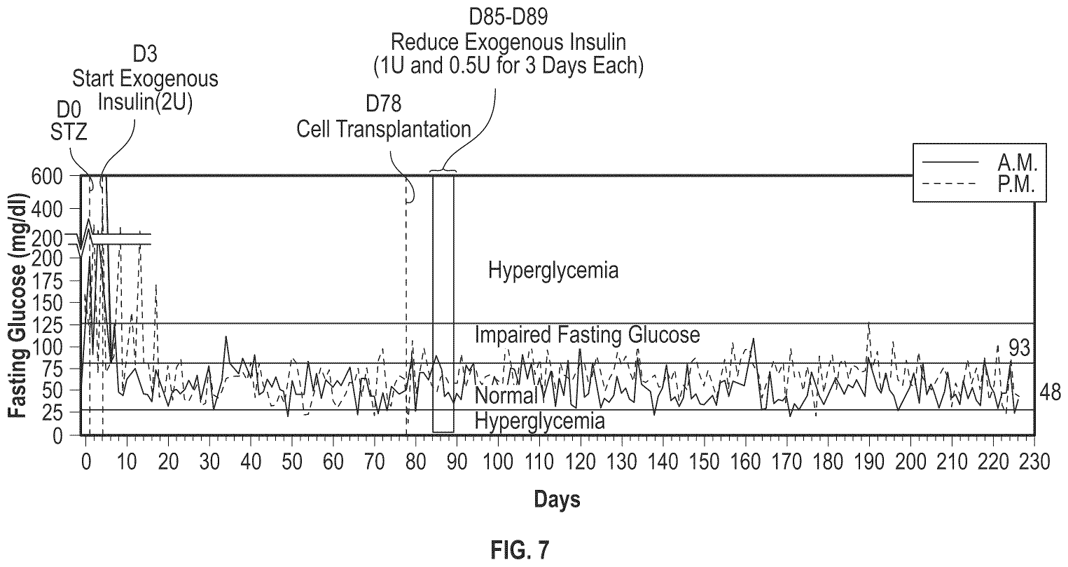

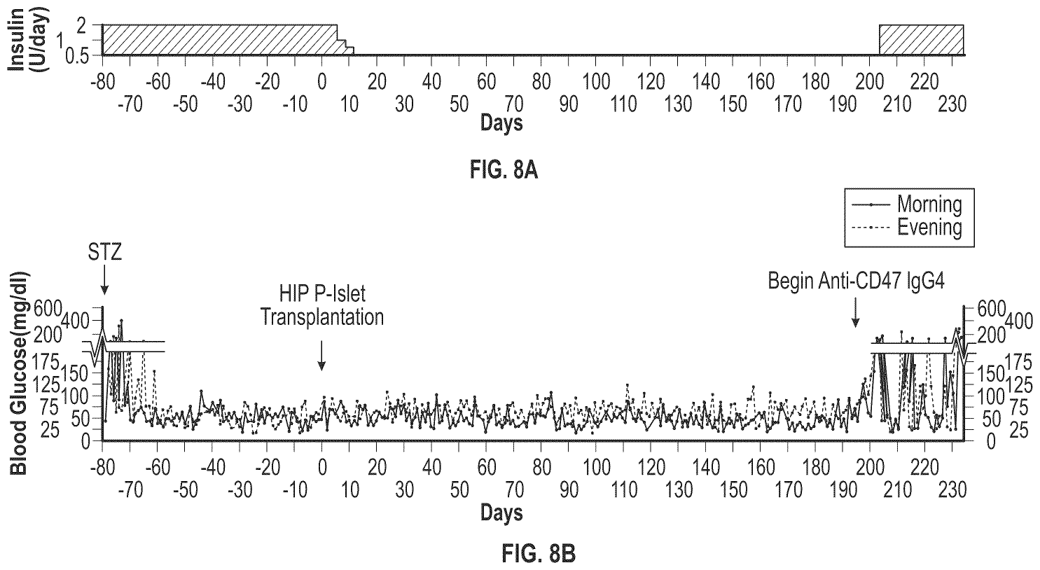

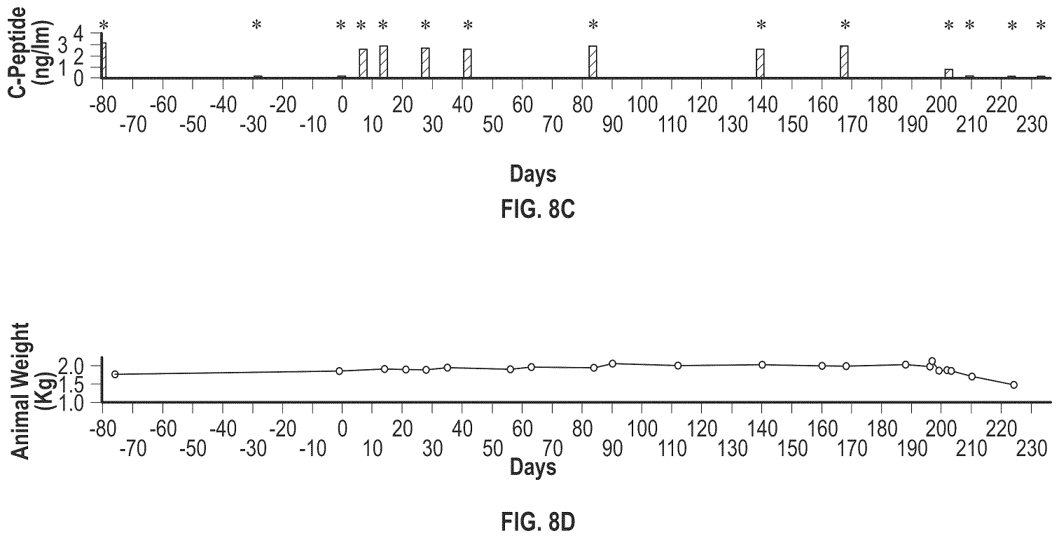

Fasting glucose was restored to normal in the diabetic NHP post islet cell transplantation. Twelve days after withdrawing insulin from the cynomolgus monkey, fasting blood glucose was maintained at normal levels in both morning (52 mg/dL) and afternoon (58 mg/dL) blood draws as shown in FIG. 5. As shown in FIG. 6 and FIG. 7, fasting blood glucose continued to be maintained at normal levels in both the morning and afternoon out to day 111, more than 20 days after withdrawing insulin from the cynomolgus monkey (FIG. 6) and out to day 226, which was 136 days after withdrawing insulin from cynomolgus monkey (FIG. 7). As shown in FIG. 8B, fasting blood glucose continued to be maintained at normal levels in both the morning and afternoon out to day 190, which was 181 days after withdrawing insulin from the cynomolgus monkey. See depiction of insulin administration in FIG. 8A. As shown in FIG. 8C, there was increased in vivo insulin production on both day 7 and day 14 post islet cell transplantation as measured by C-peptide, a byproduct of insulin production. Further, the cynomolgus monkey showed mild, physiologic weight gain over the course of the study (FIG. 8D).

The cynomolgus monkey showed tightly controlled morning and evening blood glucose levels for 6 months, was completely insulin-independent, continuously healthy, and exhibited no physical or behavioral abnormalities. No hyperglycemia events were recorded. The maintenance of insulin independence and stable c-peptide levels for over half a year underscored the steady endocrine function of the allograft. There were no local irritations from transplantation, no behavioral or laboratory abnormalities, and the monkey was healthy.

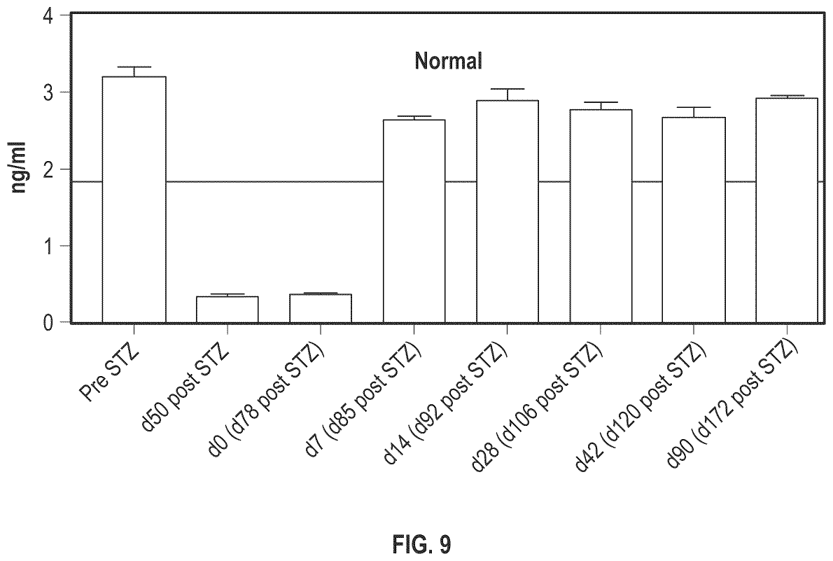

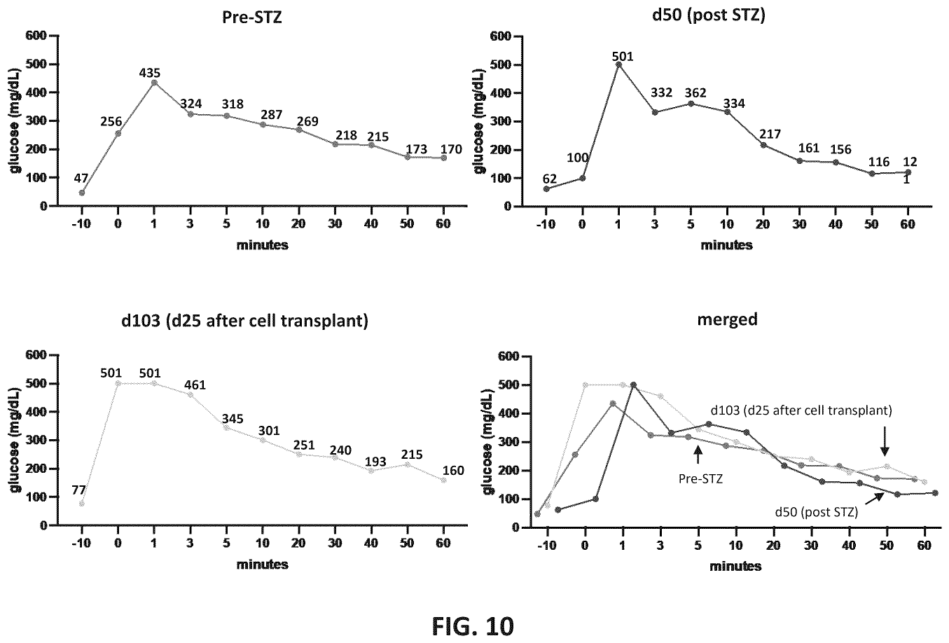

In vivo insulin production in FIG. 9 was increased post islet cell transplantation (i.e., “d7 (d85 post STZ)”, “d14 (d92 post STZ)”, “d28 (d106 post STZ)”, “d42 (d120 post STZ)”, and “d90 (d172 post STZ)”) relative to the insulin production on day 50 post STZ (“d50 post STZ”) and the day of islet cell transplantation (“d0 (d78 post STZ)”). FIG. 10 shows glucose tolerance pre-STZ administration, on day 50, and on day 103. There was no observable difference between response to glucose among the groups.

These results support the surprising finding that subjects with diabetes treated by intramuscular injection with a single relatively low dose of engineered hypoimmune islet cells can achieve insulin independence. These data indicate insulin independence in the cynomolgus monkey, which corresponds with increased in vivo insulin production on both day 7 and day 14 post islet cell transplantation (or day 92 of the study) as measured by C-peptide, a byproduct of insulin production (FIG. 8C). C-peptide proved to be a very robust marker for graft performance and can be used in a clinical setting.

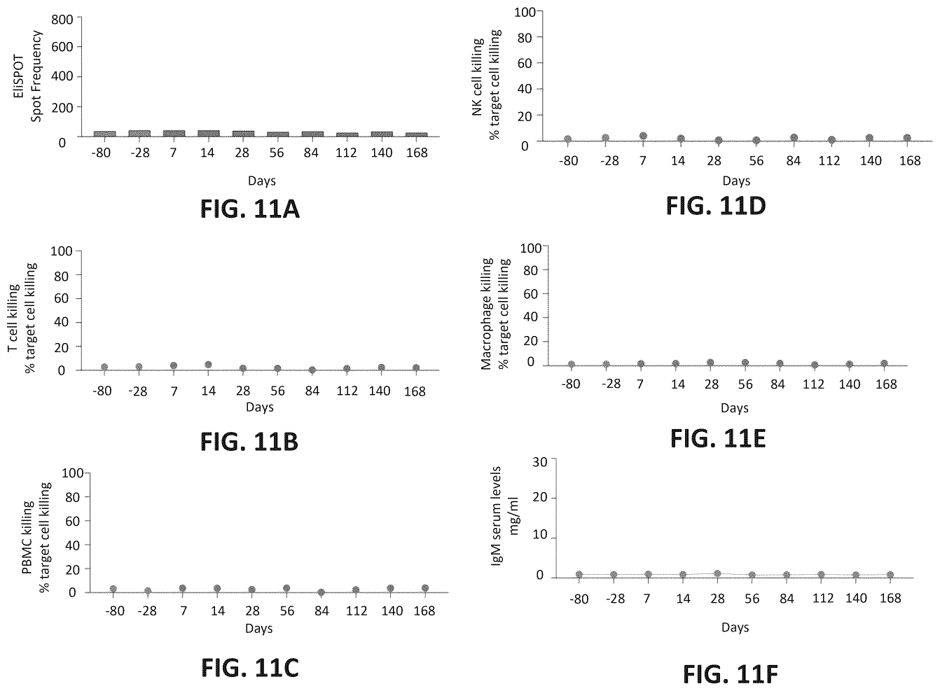

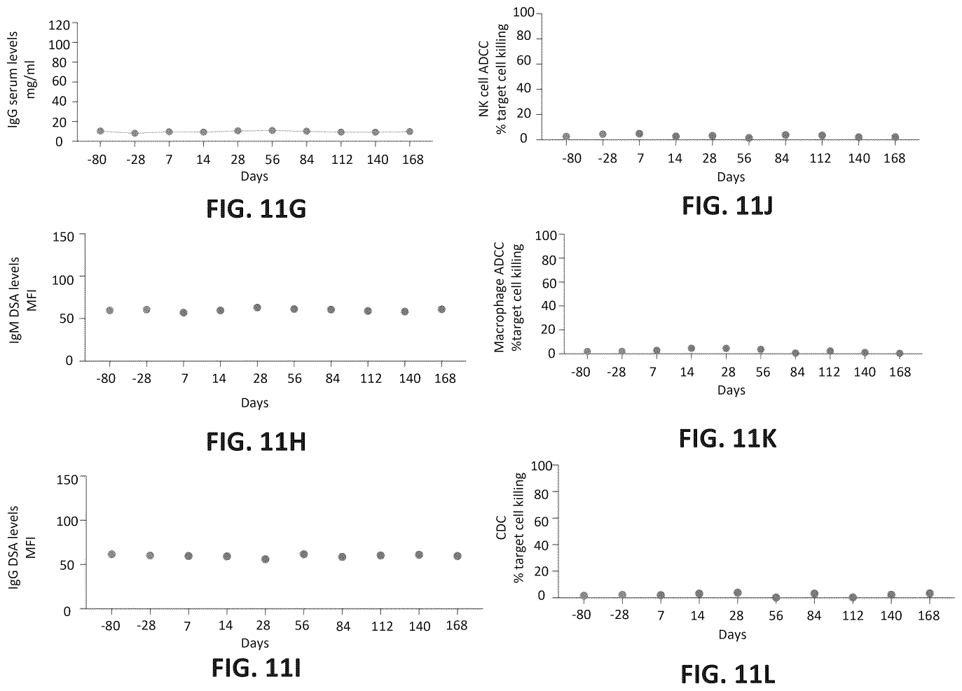

Engineered primary beta cell islets did not induce immune recognition or any immune response. Repeated immune analyses against rhesus macaque B2M^{-/-}; CIITA^{-/-}; CD47tg primary islet cells were performed during the study. PBMCs and CD3+ T cells were isolated. NK cells and macrophages were generated from PBMCs. Elispot assays with PBMCs showed no T cell activation (FIG. 11A). Cytotoxicity assays with T cells showed no killing activity against the allogeneic rhesus macaque B2M^{-/-}; CIITA^{-/-}; CD47tg primary islet cells (FIG. 11B). PBMCs, NK cells, and macrophages also showed no killing activity against allogeneic rhesus macaque B2M^{-/-}; CIITA^{-/-}; CD47tg primary islet cells (FIGS. 11C-11E).

Total IgM and IgG serum levels were quantified as surrogate markers for induced antibody production. Total serum IgM (FIG. 11F) and IgG (FIG. 11G) levels did not increase. No DSAs were detected at any time point during this study (FIGS. 11H and 11I). There were no ADCC reactions with NK cells (FIG. 11J) or macrophages (FIG. 11K), and there was no CDC against rhesus macaque B2M^{-/-}; CIITA^{-/-}; CD47tg primary islet cells (FIG. 11L). Repeated and thorough immune analyses showed no immune activation and no immune response was detected at any timepoint during the follow-up. These data demonstrate that the allogeneic rhesus macaque B2M^{-/-}; CIITA^{-/-}; CD47tg primary islet cell graft completely circumvented allograft rejection, did not build immune memory, and thus achieved markedly better protection than systemic immunosuppression.

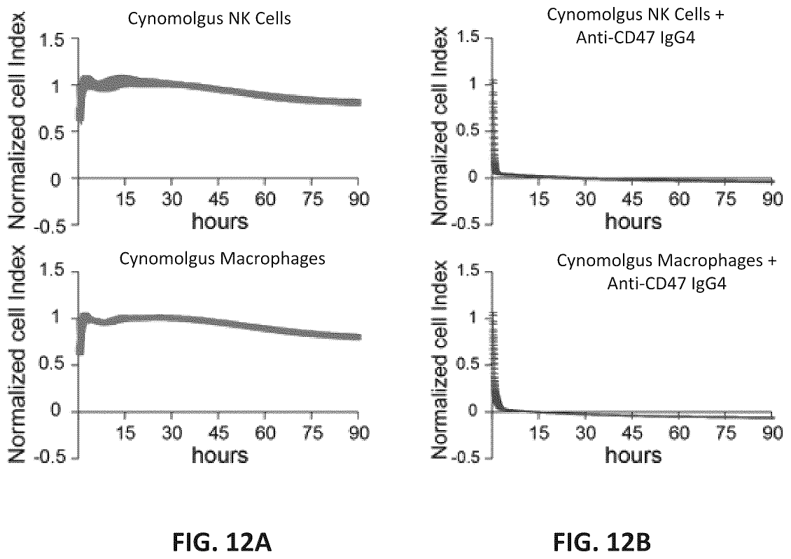

Anti-CD47 antibody acts as a safety switch to cause the death of the engineered islet cells. In order to demonstrate that the observed insulin-independence was fully dependent on the well-functioning islet graft and that there was no regeneration or recovery of the endogenous islet cell population, isolated rhesus macaque B2M^{-/-}; CIITA^{-/-}; CD47tg primary islet cells were treated with a CD47 antibody (magrolimab). Humanized anti-CD47 IgG4 antibody blocks the interaction of rhesus CD47 with cynomolgus SIRPα. The B2M^{-/-}; CIITA^{-/-}; CD47tg edited rhesus macaque primary islet cells rely on CD47 expression to evade cell killing by NK cells and macrophages. Once CD47 is blocked, the edited islet cells are recognized as MHC class I and II double negative cells and undergo innate immune cell killing. FIG. 12A shows that the edited rhesus macaque primary cells evade cell killing by cynomolgus NK cells and macrophages. FIG. 12B shows that the edited rhesus macaque primary cells are killed by cynomolgus NK cells and macrophages when treated with magrolimab. Following in vivo CD47 antibody-mediated ablation of the edited rhesus macaque islet cell graft, blood glucose levels increased steadily to >127 mg/dl (FIG. 8B). Cynomolgus monkeys were also hypoactive and lethargic.

The quick relapse of diabetes requiring daily insulin injections after the CD47-targeted, antibody-driven destruction of the B2M^{-/-}; CIITA^{-/-}; CD47tg graft showed the dependence of the cynomolgus monkey on graft function. The quick relapse of diabetes also reaffirmed that STZ at appropriate doses can be used to reliably induce lasting diabetes in cynomolgus monkeys without evidence of reactive D-cell regeneration in the pancreas of the animals (Du et al., NatMed (2022) 28:272-282; Zhu et al., J Diabetes Res (2014) 785948; Dufrane et al, Transplantation (2006) 81:36-45; Saisho et al., Diabetes (2011) 60:845-856) and supported the reliability of an anti-CD47 safety switch.

On day 234, the cynomolgus monkey was euthanized and underwent necropsy. Histologic analysis of the pancreas showed a significant reduction in the numbers of insulin positive beta cells (FIG. 13B) compared to pancreas from a healthy cynomolgus monkey (FIG. 13A). However, the islet cells stained positive for glucagon and somatostatin (FIG. 13B). These data confirm the effective and selective beta cell destruction by STZ and also confirms that there is no endogenous beta cell regeneration in subsequent months post STZ treatment.

The islet injection sites in both quadriceps muscles were recovered for histologic analysis. While endogenous islets from a healthy cynomolgus monkey stained positive for islet1 (FIG. 13A), a lineage-specific marker for pancreatic neuroendocrine cells not restricted to beta cells, islet1 was not detected in the quadriceps muscle (FIG. 13C) indicating the absence of residual islet+ cells. However, there was post-inflammatory fibrosis suggestive of phagocytic events (data not shown). These data confirm that treatment with anti-CD47 IgG4 antibody completely destroys the B2M^{-/-}; CIITA^{-/-}; CD47tg primary islets.

Overall, the present Example demonstrates the successful, curative treatment of an immunocompetent, diabetic cynomolgus monkey with allogeneic, B2M^{-/-}; CIITA^{-/-}; CD47tg engineered rhesus macaque primary islet cells without concomitant administration of any immunosuppression therapy. Specifically, B2M^{-/-}; CIITA^{-/-}; CD47tg engineered rhesus macaque primary islet cells provided lasting endocrine function and cynomolgus monkeys achieved insulin independence. Given the phylogenetic proximity of NHP to humans, the present NHP islet transplantation model is an accepted model for a human engineered islet cell therapy.

### Example 3 Safety Study of Hypoimmune Pancreatic Islet Transplantation in Adult Subjects with Type 1 Diabetes

This study describes a clinical study to treat adult type 1 diabetes subjects with a drug product comprising hypoimmune human primary cadaveric beta islet cells (HPC-beta islet DP). HPC-beta islet DP is composed of human pancreatic islets obtained from regular deceased organ donors that are genetically modified to evade allogeneic immune cell recognition. Isolated human pancreatic islets from donor subjects are engineered as described in Example 3 by transducing with a lentiviral vector (LVV) to incorporate the gene for overexpression of CD47, and gene editing with a Clustered Regularly Interspaced Short Palindromic Repeats (CRISPR)/CRISPR associated protein (Cas) 12b system to abrogate the function of beta-2-microglobulin (B2M) and the class II major histocompatibility complex transactivator (CIITA) genes. The overexpression of CD47 is key for evasion from cells of the innate immune system. The impairment of HLA I/II function through knockout of B2M and CIITA genes via CRISPR/Cas12b system serves the evasion from cells of the adaptive immune system. Together, the aim is to enhance survival and persistence of the transplanted genetically modified islets.

The engineered HPC-beta islet DP is prepared as a suspension of fresh (non-cryopreserved) pancreatic islets, which are formulated for intramuscular injection into a patient.

HPC-beta islet DP is intended for the treatment of type 1 diabetes as an approach for clinical islet transplantation without the need of long-term systemic immunosuppression in the intramuscular location (forearm). The clinical study is designed in order to evaluate safety and the ability to restore insulin production without the need of simultaneous immunosuppression. In some cases, immunosuppression is administered to the subject. Secondary objectives include studying changes in islet-cell function, metabolic control, diabetes treatment satisfaction, and immunological response to pancreatic islets during the first year following treatment.

### A. Inclusion Criteria

1. Subjects eligible for inclusion in this study meet the following criteria:
Diagnosis of type 1 diabetes mellitus (T1D):
i) for >5 years and
ii) diagnosed before the age of 18 years and
iii) at least one or more HbA1c documented in the subject's medical journal or Swedish
2. National Diabetes Registry during the last five-year period must be >70 mmol/mol.
3. The subject must be involved in intensive diabetes management defined as self-monitoring of subcutaneous glucose level by continuous glucose monitoring or by intermittent scanning glucose monitoring no less than a mean of three times per day averaged over each week and by the administration of three or more insulin injections per day or insulin pump therapy.
4. C-peptide negative (C-peptide <0.01 nmol/1) in response to a mixed meal tolerance test MMTT).
5. Positive for antibodies to either GAD or IA2 at screening.
6. 30-45 years of age at time of enrollment.
7. HbA1c≥70 mmol/mol.
8. Exogenous insulin needs <1 IU/kg.
9. Body weight <80 kg.
10. Female subjects of child-bearing potential must agree to using adequate contraception until one year after the administration of HPC-beta islet DP.
11. Male subjects must not intend to procreate until one year after the administration of HPC-beta islet DP. Males must be willing to use effective measures of contraception (condoms) during the one-year trial period.

### B. Exclusion Criteria

Subjects fulfilling any of the following criteria at screening are not eligible for inclusion in this study.
1. Any previous organ transplantation.
2. Any systemic immunosuppressive medication for any other disease.
3. Any history of malignancy.
4. Use of any investigational agent(s) within 4 weeks of enrollment.
5. Use of any anti-diabetic medication, other than insulin, within 4 weeks of enrollment.
6. Active infections including Tuberculosis, HIV, HBV and HCV.
7. Liver function test value for AST, ALT, GGT or ALP exceeding the respective reference interval for the clinical assay at Uppsala university hospital.
8. Increased plasma concentrations of alanine aminotransferase (>0.75 katl/l for females or >1.1 kat/l for males) and/or aspartate aminotransferase (>0.60 kat/l for females or >0.75 kat/l for males), and/or y-glutamyl transferase (>0.75 kat/l for females 18-40 years of age or >1.2 kat/l for females >40 years or >1.3 kat/l for males >40 years or >1.9 kat/l for males >40 years) and/or alkaline phosphatase (>1.8 kat/l).
9. Serological evidence of infection with HTLVI or HTLVII.
10. Pregnancy, nursing, intention for pregnancy.
11. Chronic kidney disease grade 3 or worse (GFR<60 ml/min as estimated by creatine measurement).
12. Medical history of cardiac disease, or symptoms at screening consistent with cardiac disease.
13. HLA immunization.
14. MIC A/B immunization.
15. Known autoimmune disease other than type I diabetes (e.g., Hashimoto disease).
16. Administration of live attenuated vaccines <6 months before transplant.
17. Islet antibodies where GADA >2000 IE/ml or IA2A >4000 IE/ml, or positive for ZnT8 auto-antibodies.
18. Untreated proliferative diabetic retinopathy.
19. Major ongoing psychiatric illness which the Principal Investigator judge increases the risk of noncompliance or does not allow safe participation in the study.
20. Ongoing substance abuse, drug or alcohol; or recent history of noncompliance.
21. Known hypersensitivity to ciprofloxacin, gentamicin, or amphotericin (since these are used in the manufacturing process of HPC-beta islet DP and trace amounts may be present in final product).

### C. Administration and Dosing of HPC-Beta Islet DP

Human subjects with Type I diabetes included in the study are administered an islet cell suspension of the HP-beta islet DP by intramuscular injection to the forearm at a dose of from about 25×10⁶-80×10⁶ islet cells.

The study groups may be further administered an additional therapy (e.g., combination therapy), including but not limited to: (i) re-dosing of the same or different cells; (ii) pre-treatment, concurrent treatment, and/or subsequent treatment with an additional agent (e.g., biologic, small molecule, or any combination thereof); (iii) pre-conditioning agents; and (iv) associated regimens.

A cohort of subjects are administered the HP-beta islet DP without immunosuppression. A cohort of subjects are administered the HP-beta islet DP with immunosuppression. Immunosuppression as used in this study may include, but is not limited to, immunosuppression protocols known in the art which are useful to prevent rejection of adoptively transferred allogeneic donor material, such as cells, tissue, organs, etc. by a recipient. Protocols of particular relevance to this study include those used with islet cell transplant (e.g., primary beta islet cells) or other pancreatic-derived tissues or cells. Non-limiting examples of immunosuppressive protocols, immunosuppression agents, and the like for use in the present study include rabbit antithymocyte globulin (ATG), etanercept, basiliximab, cyclophosphamide, fludarabine, calcineurin-based agents, Edmonton protocol or modifications thereof (anti-IL-2Ra monoclonal antibody (daclizumab), cyclosporine, tacrolimus, MMF, sirolimus (rapamycin), leflunomide, prednisone.

Exemplary immunosuppressive protocols are performed as described in: Markmann et al. “Phase 3 trial of human islet-after-kidney transplantation in type 1 diabetes.” Am J Transplant. 2021; 21(4): 1477-1492; Shapiro et al. “Clinical pancreatic islet transplantation.” Nature Reviews. Endocrinology. 2017; 13(5):268-277; Hering et al. “Phase 3 trial of transplantation of human islets in type 1 diabetes complicated by severe hypoglycemia.” Diabetes Care. 2016; 39(7):1230-1240; Foster et al. “Clinical Islet Transplantation Consortium. Improved health-related quality of life in a phase 3 islet transplantation trial in type 1 diabetes complicated by severe hypoglycemia.” Diabetes Care. 2018. pii:dc171779. doi:10.2337/dc17-1779; Korsgren et al. “Current status of clinical islet transplantation.” Transplantation 2005; 79: 1289-1293; Shapiro et al. “Islet transplantation in seven patients with type 1 diabetes mellitus using a glucocorticoid-free immunosuppressive regimen.” N. Engl. J. Med. 2000; 343:230-238; and NIAID. “Islet transplantation in type 1 diabetes” [ClinicalTrials.gov study NCT00434811].

Immunosuppression tapering protocols and use of two or more immunosuppression protocols, such as changing from a first immunosuppression protocol to a second immunosuppression protocol, are also compatible with this study. Such change could occur, for example, while the hypoimmune allogeneic primary beta islet cells are already transplanted into the patient or prior to the transplantation.

The immunosuppression regimen includes induction therapy with Basiliximab (2×20 mg iv) followed by Tacrolimus (start dose 0.1 mg/kg/24 h; with target concentration of 10-12 mg/kg/24 h and MMF immunosuppression (500 mg 2×2, dose adjusted thereafter based on AUC), as well as CMV prophylaxis with Valganciclovir 450 mg 2×1, ulcer prophylaxis with omeprazole 20 mg 1×1, TNF-alpha inhibition with etanercept (50 mg iv, followed by 25 mg sc on day 3, 7 and 10) and standard antibiotics.

All subjects receive a concomitant therapy with standard insulin treatment subcutaneously. Insulin treatment is prescribed per routine clinical practice. Study participants do not, if not having a clinical indication for such specific treatment, receive concomitant medication that may interfere with glucose regulation other than insulin treatments, e.g. oral anti-diabetic therapies, GLP-1 RA's, or any systemic immunosuppressive treatments. Subjects are allowed to take other medications that do not interfere with glucose regulation.

### D. Safety and Response Assessment

Primary endpoints of the study include safety assessment, while response and efficacy are assessed as secondary endpoints.

### 1. Safety

Safety parameters are evaluated and are recorded as adverse events (AEs). The primary outcome measure is number of treatment-related adverse events as assessed by CTCAE v5.0 (Common Terminology Criteria for Adverse Events (CTCAE) Version 5.0. U.S. Department of Health and Human Services National Institutes of Health National Cancer Institute. Available at https://www.meddra.org/(Cited Nov. 27, 2017). Safety is monitored for 12 months follow up after transplantation of HPC-beta islet DP. Adverse events are classified with regard to seriousness, expectedness and relatedness to the investigational product. Any occurring adverse event is handled according to clinical routine.

Criteria for success of first subject after 12 weeks include no AEs that are serious adverse events (SAEs related to treatment or study procedure (e.g., CTCAE grade 3 or higher)).

### 2. Response and Efficacy

Response to the HPC-beta islet DP is evaluated by assessment of parameters associated with immune evasion and parameters associated with treatment of diabetes.

Clinical Chemistry. Routine lab parameters are measured during the study at a clinical chemistry laboratory, including CRP, hematocrit, hemoglobin, red blood cells, platelets, white blood cells, liver status (AST, ALT, ALP, GGT, bilirubin), PK, PTT, D-dimer, B-HCG, sodium, potassium, P-glucose, C-peptide, HbA1c. In order to avoid false negative results, C-peptide concentrations are also analyzed with an ultrasensitive C-peptide ELISA (Mercodia©: Cat. No. 10-1141-01). The assay has been calibrated against the International Reference Reagent for C-peptide (C-peptide 84/510; a WHO standard). The detection limit of the ultrasensitive C-peptide from Mercodia is 1.167-130 pmol/l, with inter- and intra-assay CVs at 5.5 and 3.8% at 37 pmol/l. All plasma samples investigated for C-peptide will be analyzed as duplicates. Samples with a CV-value >15% are excluded.

Mixed Meal Tolerance Test. Measurement of endogenous insulin production as C-peptide concentrations in response to a mixed meal tolerance test is an important efficacy measure of the present study. Efficacy of treatment is measured as change in this parameter when compared to before the start of treatment. After an overnight fast, the subject performs a standardized mixed meal tolerance test, wherein the subjects drink a solution of resource protein (Novartis, 6 ml/kg, maximal dose 360 ml). Blood samples are taken for analysis at time-point 0, 15, 30, 60, 90, and 120 minutes. In peripheral venous blood plasma from every time-point, C-peptide values are measured, and AUC C-peptide concentration calculated for the test.

Glucose Variability. Intermittent scanning glucose monitoring (isCGM) is performed. For individuals with no ongoing CGM/isCGM by their own device, a subject-blinded system is used. For those that have isCGM already, these values can be used. For those that have CGM, an open isCGM is used, since their own CGM already provides them with continuous data. isCGM will, at each evaluation, be used for 14 days and used for calculation of time-in-range 3.9-10 mmol/l.

Measurements of HbA1c. Measurements of HbA1c is performed to assess metabolic control during the study.

Specifically, secondary efficacy endpoints include the following:
1. Immune evasion of implanted cells, as evaluated in systemic PBMC and serum at 0 (before transplantation), 2, 4, 8, 12-, 18-, 26- and 52-weeks post-transplantation.
2. Presence of peak c-peptide >0.01 nmol/l, in response to the mixed meal tolerance test at 4, 8, 12,18 and 26- and 52-weeks post-transplantation.
3. Presence of non-fasting c-peptide concentrations >0.01 nmol/1 at 1, 2, 3, 4, 6, 8, 10, 12, 14, 16, 18, 20, 22-, 24-, 26- and 52-weeks post-transplantation; i.e. Kaplan-Meier analysis of survival time for islet grafts.
4. Presence of peak c-peptide >0.20 nmol/l, in response to the mixed meal tolerance test at 4, 8, 12,18 and 26- and 52-weeks post-transplantation.
5. Survival of implanted cells, as evaluated by MRI, within 48 h after transplant and 2, 4, 6, 8-, 12-, 26- and 52-weeks post-transplantation.
6. C-peptide AUC in response to the mixed meal tolerance test at 4, 8, 12, 18 and 26- and 52-weeks post-transplantation.
7. Delta changes in insulin requirement/kg BW at 1, 2, 3, 4, 6, 8, 10, 12, 14, 16, 18, 20, 22-, 24-, 26- and 52-weeks post-transplantation when compared to before transplantation.
8. Delta changes in HbA1c at 2, 4, 6, 8, 10, 12, 14, 16, 18-, 20-, 26- and 52-weeks post-transplantation when compared to before transplantation.
9. Delta changes in glucose variability (time in range, SD, CV) and hypo/hyperglycemia duration derived from a continuous glucose monitoring system performed at 4, 8, 12-, 18-, 26- and 52-weeks post-transplantation when compared to before transplantation.
10. Score in diabetes treatment satisfaction questionnaire in transplanted subjects at 4, 8, 12-, 18-, 26- and 52-weeks post-transplantation when compared to before transplantation.

Criteria for success of first patient after 12 weeks includes a mixed meal tolerance test that shows measurable C-peptide >0.01 nmol/1 at any time between 4-12 weeks.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| # | SEQUENCE | ANNOTATION |
|---|---|---|
| 1 | QLLFNKTKSVEFTFCNDTVVIPCFVTNMEAQNTTEVYVKWKFKG | Human CD47; aa |
| | RDIYTFDGALNKSTVPTDFSSAKIEVSQLLKGDASLKMDKSDAVS | 19-323 |
| | HTGNYTCEVTELTREGETIIELKYRVVSWFSPNENILIVIFPIFAILL | |
| | FWGQFGIKTLKYRSGGMDEKTIALLVAGLVITVIVIVGAILFVPGE | |
| | YSLKNATGLGLIVTSTGILILLHYYVFSTAIGLTSFVIAILVIQVIAYI | |
| | LAVVGLSLCIAACIPMHGPLLISGLSILALAQLLGLVYMKFVASNQ | |
| | KTIQPPRKAVEEPLNAFKESKGMMNDE | |
| 2 | MWPLVAALLLGSACCGSAQLLENKTKSVEFTFCNDTVVIPCFVTN | Human CD47; aa |
| | MEAQNTTEVYVKWKFKGRDIYTFDGALNKSTVPTDFSSAKIEVS | 1-323 |
| | QLLKGDASLKMDKSDAVSHTGNYTCEVTELTREGETIIELKYRVV | |
| | SWFSPNENILIVIFPIFAILLFWGQFGIKTLKYRSGGMDEKTIALLV | |
| | AGLVITVIVIVGAILFVPGEYSLKNATGLGLIVTSTGILILLHYYVFS | |
| | TAIGLTSFVIAILVIQVIAYILAVVGLSLCIAACIPMHGPLLISGLSIL | |
| | ALAQLLGLVYMKFVASNQKTIQPPRKAVEEPLNAFKESKGMMN | |
| | DE | |
| 3 | CEEPPTFEAMELIGKPKPYYEIGERVDYKCKKGYFYIPPLATHTIC | Human CD46, aa |
| | DRNHTWLPVSDDACYRETCPYIRDPLNGQAVPANGTYEFGYQM | 35-392 |
| | HFICNEGYYLIGEEILYCELKGSVAIWSGKPPICEKVLCTPPPKIKN | |
| | GKHTFSEVEVFEYLDAVTYSCDPAPGPDPFSLIGESTIYCGDNSVW | |
| | SRAAPECKVVKCRFPVVENGKQISGFGKKFYYKATVMFECDKGF | |
| | YLDGSDTIVCDSNSTWDPPVPKCLKVLPPSSTKPPALSHSVSTSST | |
| | TKSPASSASGPRPTYKPPVSNYPGYPKPEEGILDSLDVWVIAVIVIA | |
| | IVVGVAVICVVPYRYLQRRKKKGTYLTDETHREVKFTSL | |
| 4 | MEPPGRRECPFPSWRFPGLLLAAMVLLLYSFSDACEEPPTFEAME | Human CD46; aa |
| | LIGKPKPYYEIGERVDYKCKKGYFYIPPLATHTICDRNHTWLPVS | 1-392 |
| | DDACYRETCPYIRDPLNGQAVPANGTYEFGYQMHFICNEGYYLI | |
| | GEEILYCELKGSVAIWSGKPPICEKVLCTPPPKIKNGKHTFSEVEVF | |
| | EYLDAVTYSCDPAPGPDPFSLIGESTIYCGDNSVWSRAAPECKVV | |
| | KCRFPVVENGKQISGFGKKFYYKATVMFECDKGFYLDGSDTIVC | |
| | DSNSTWDPPVPKCLKVLPPSSTKPPALSHSVSTSSTTKSPASSASGP | |
| | RPTYKPPVSNYPGYPKPEEGILDSLDVWVIAVIVIAIVVGVAVICV | |
| | VPYRYLQRRKKKGTYLTDETHREVKFTSL | |
| 5 | LQCYNCPNPTADCKTAVNCSSDFDACLITKAGLQVYNKCWKFEH | Human CD59; aa |
| | CNFNDVTTRLRENELTYYCCKKDLCNFNEQLENGGTSLSEKTVL | 26-128 |
| | LLVTPFLAAAWSLHP | |
| 6 | MGIQGGSVLFGLLLVLAVFCHSGHSLQCYNCPNPTADCKTAVNC | Human CD59; aa |
| | SSDFDACLITKAGLQVYNKCWKFEHCNFNDVTTRLRENELTYYC | 1-128 |
| | CKKDLCNFNEQLENGGTSLSEKTVLLLVTPFLAAAWSLHP | |
| 7 | LQCYNCPNPTADCKTAVNCSSDFDACLITKAGLQVYNKCWKFEH | Mature Human |
| | CNFNDVTTRLRENELTYYCCKKDLCNFNEQLEN | CD59; aa 26-102 |
| 8 | DCGLPPDVPNAQPALEGRTSFPEDTVITYKCEESFVKIPGEKDSVI | Human CD55; aa |
| | CLKGSQWSDIEEFCNRSCEVPTRLNSASLKQPYITQNYFPVGTVV | 35-381 |
| | EYECRPGYRREPSLSPKLTCLQNLKWSTAVEFCKKKSCPNPGEIR | |
| | NGQIDVPGGILFGATISFSCNTGYKLFGSTSSFCLISGSSVQWSDPL | |
| | PECREIYCPAPPQIDNGIIQGERDHYGYRQSVTYACNKGFTMIGEH | |
| | SIYCTVNNDEGEWSGPPPECRGKSLTSKVPPTVQKPTTVNVPTTE | |
| | VSPTSQKTTTKTTTPNAQATRSTPVSRTTKHFHETTPNKGSGTTSG | |
| | TTRLLSGHTCFTLTGLLGTLVTMGLLT | |
| 9 | MTVARPSVPAALPLLGELPRLLLLVLLCLPAVWGDCGLPPDVPN | Human CD55; aa |
| | AQPALEGRTSFPEDTVITYKCEESFVKIPGEKDSVICLKGSQWSDIE | 1-381 |
| | EFCNRSCEVPTRLNSASLKQPYITQNYFPVGTVVEYECRPGYRRE | |
| | PSLSPKLTCLQNLKWSTAVEFCKKKSCPNPGEIRNGQIDVPGGILF | |
| | GATISFSCNTGYKLFGSTSSFCLISGSSVQWSDPLPECREIYCPAPP | |
| | QIDNGIIQGERDHYGYRQSVTYACNKGFTMIGEHSIYCTVNNDEG | |
| | EWSGPPPECRGKSLTSKVPPTVQKPTTVNVPTTEVSPTSQKTTTKT | |
| | TTPNAQATRSTPVSRTTKHFHETTPNKGSGTTSGTTRLLSGHTCFT | |
| | LTGLLGTLVTMGLLT | |
| 10 | DCGLPPDVPNAQPALEGRTSFPEDTVITYKCEESFVKIPGEKDSVI | Mature Human |
| | CLKGSQWSDIEEFCNRSCEVPTRLNSASLKQPYITQNYFPVGTVV | CD55; aa 35-353 |
| | EYECRPGYRREPSLSPKLTCLQNLKWSTAVEFCKKKSCPNPGEIR | |
| | NGQIDVPGGILFGATISFSCNTGYKLFGSTSSFCLISGSSVQWSDPL | |
| | PECREIYCPAPPQIDNGIIQGERDHYGYRQSVTYACNKGFTMIGEH | |
| | SIYCTVNNDEGEWSGPPPECRGKSLTSKVPPTVQKPTTVNVPTTE | |
| | VSPTSQKTTTKTTTPNAQATRSTPVSRTTKHFHETTPNKGSGTTS | |
| 11 | EGRGSLLTCGDVEENPGP | T2A |
| 12 | LEGGGEGRGSLLTCGDVEENPGPR | T2A |
| 13 | GSGATNFSLLKQAGDVEENPGP | P2A |
| 14 | ATNFSLLKQAGDVEENPGP | P2A |
| 15 | QCTNYALLKLAGDVESNPGP | E2A |
| 16 | VKQTLNFDLLKLAGDVESNPGP | F2A |
| 17 | GSGEGRGSLLTCGDVEENPGP | T2A |
| 18 | AAGSGEGRGSLLTCGDVEENPGP | T2A |
| 19 | GUUUUAGAGCUA | Exemplary |
| | | crRNA repeat |
| 20 | UAGCAAGUUAAAAUAAGGCUAGUCCGUUAUCAACUUGAAAA | Exemplary |
| | AGUGGCACCGAGUCGGUGCUUU | tracrRNA |
| 21 | GAAA | Tetraloop |
| | | sequence |
| 22 | ACCCCACAGTGGGGCCACTA | CCR5 gRNA |
| 23 | TGTTGGAAGGATGAGGAAAT | CCR5 gRNA |
| 24 | TCACTATGCTGCCGCCCAGT | CCR5 gRNA |
| 25 | UCUCUCCAUGUGCAGUAGGA | ABO gRNA |
| 26 | CUGGAUGUCGGAGGAGUACG | FUT1 gRNA |
| 27 | GUCUCCGGAAACUCGAGGUG | RHD gRNA |
| 28 | ACAGUGUAGACUUGAUUGAC | F3 (CD142) |
| | | gRNA |
| 29 | CGUGAGUAAACCUGAAUCUU | macaca mulatta |
| | | B2M gene gRNA |
| | | sequence |
| 30 | GAUAUUGGCAUAAGCCUCCC | macaca mulatta |
| | | CIITA gene |
| | | gRNA sequence |
| 31 | AGAGUCUCUCAGCUGGUACA | TRAC gRNA |
| 35 | MWPLVAALLLGSACCGSA | CD47 SP domain |
| 36 | QLLFNKTKSVEFTFCNDTVVIPCFVTNMEAQNTTEVYVKWKFKG | CD47 IgV |
| | RDIYTFDGALNKSTVPTDFSSAKIEVSQLLKGDASLKMDKSDAVS | domain |
| | HTGNYTCEVTELTREGETII | |
| 37 | QLLFNKTKSVEFTFCNDTVVIPCFVTNMEAQNTTEVYVKWKFKG | CD47 EC domain |
| | RDIYTFDGALNKSTVPTDFSSAKIEVSQLLKGDASLKMDKSDAVS | |
| | HTGNYTCEVTELTREGETIIELKYRVVSWFSPNE | |
| 38 | NILIVIFPIFAILLFWGQFGI | CD47 TM1 |
| | | domain |
| 39 | KTLKYRSGGMDEKT | CD47 IC1 domain |
| 40 | IALLVAGLVITVIVIVGAILF | CD47 TM2 |
| | | domain |
| 41 | VPGEYSLKNA | CD47 EC1 |
| | | domain |
| 42 | TGLGLIVTSTGILILLHYYVF | CD47 TM3 |
| | | domain |
| 43 | STAIGLT | CD47 IC2 domain |
| 44 | SFVIAILVIQVIAYILAVVGL | CD47 TM4 |
| | | domain |
| 45 | SLCIAACIPMHG | CD47 EC2 |
| | | domain |
| 46 | PLLISGLSILALAQLLGLVYM | CD47 TM5 |
| | | domain |
| 47 | KFVASNQKTIQPPRKAVEEPLNAFKESKGMMNDE | CD47(202) IC3 |
| | | domain |
| 48 | KFVASNQKTIQPPRNN | CD47(201) IC3 |
| | | Domain |
| 49 | PNKGSGTTSGTTRLLSGHTCFTLTGLLGTLVTMGLLT | DAF/CD55 GPI |
| 50 | ATVETPAAEETMNTSPGTPAPAAEETMNTSPGTPAPAAEETMTTS | TRAILR3 GPI |
| | PGTPAPAAEETMTTSPGTPAPAAEETMITSPGTPASSHYLSCTIVGI | Long (with TAPE |
| | IVLIVLLIVFV | repeats) |
| 51 | MITSPGTPASSHYLSCTIVGIIVLIVLLIVFV | TRAILR3 GPI |
| | | Short (without |
| | | TAPE repeats) |
| 52 | QLENGGTSLSEKTVLLLVTPFLAAAWSLHP | CD59 GPI |
| 53 | IYIWAPLAGTCGVLLLSLVIT | CD8a TM domain |
| 54 | LYCNHRNRR | CD8a 9AA ICD |
| 55 | FWVLVVVGGVLACYSLLVTVAFIIFWV | CD28 TM domain |
| 56 | RSKRSRLLH | CD28 9AA ICD |
| 57 | VVISAILALVVLTIISLIILI | PDGFR TM |
| | | domain |
| 58 | MLWQKKPRY | PDGFR 9AA ICD |
| 59 | ELK | CD47 3AA Hinge |
| | | domain |
| 60 | TTTPAPRPPTPAPTIASQPLSLRPEACRPAAGGAVHTRGLDFACD | CD8a Hinge |
| | | domain |
| 61 | IEVMYPPPYLDNEKSNGTIIHVKGKHLCPSPLFPGPSKP | CD28 Hinge |
| | | domain |
| 62 | AVGQDTQEVIVVPHSLPFKV | PDGFR Hinge |
| | | domain |
| 63 | ESKYGPPCPPCP | IgG4 Hinge |
| | | domain |
| 64 | EVQLQQSGAELVKPGASVKLSCTASGFNIKDYYIHWVQQRTEQG | VH or HC |
| | LEWIGRIDPEDGETKYAPKFQDKATITADTSSNTAYLHLSSLTSED | |
| | TAVYYCARWGAYWGQGTLVTVSA | |
| 65 | QIVLTQSPAIMSASPGEKVTLTCSASSSVSSSYLYWYQQKPGSSPK | VL or LC |
| | LWIYSTSNLASGVPARFSGSGSGTSYSLTISSMEAEDAASYFCHQ | |
| | WSSYPRTFGAGTKLELK | |
| 66 | EVQLQQSGAELVKPGASVKLSCTASGFNIKDYYIHWVQQRTEQG | VH or HC |
| | LEWIGRIDPEDGETKYAPKFQDKATITADTSSNTAYLHLSSLTSED | |
| | TAVYYCARWGAYWGQGTLVTVSS | |
| 67 | EVQLQQSGAELVKPGASVKLSCTASGFNIKDYYIHWVQQRTEQG | scFv |
| | LEWIGRIDPEDGETKYAPKFQDKATITADTSSNTAYLHLSSLTSED | |
| | TAVYYCARWGAYWGQGTLVTVSGGGGSGGGGSGGGGSQIVLT | |
| | QSPAIMSASPGEKVTLTCSASSSVSSSYLYWYQQKPGSSPKLWIYS | |
| | TSNLASGVPARFSGSGSGTSYSLTISSMEAEDAASYFCHQWSSYP | |
| | RTFGAGTKLELK | |
| 68 | QIVLTQSPAIMSASPGEKVTLTCSASSSVSSSYLYWYQQKPGSSPK | scFv |
| | LWIYSTSNLASGVPARFSGSGSGTSYSLTISSMEAEDAASYFCHQ | |
| | WSSYPRTFGAGTKLELKGTTAASGSSGGSSSGAEVQLQQSGAEL | |
| | VKPGASVKLSCTASGFNIKDYYIHWVQQRTEQGLEWIGRIDPEDG | |
| | ETKYAPKFQDKATITADTSSNTAYLHLSSLTSEDTAVYYCARWG | |
| | AYWGQGTLVTVS | |
| 69 | EVQLVQSGAEVKKPGESLRISCKASGYTFTSYWVHWVRQMPGK | VH or HC |
| | GLEWIGNIDPSDSDTHYNQKFKDHVTLSVDKSISTAYLQLSSLKA | |
| | SDTAMYYCVRGGTGTMAWFAYWGQGTLVTVSS | |
| 70 | DVVMTQSPLSLPVTLGQPASISCRSSQSLVHSYGNTYLYWYQQRP | VL or LC |
| | GQSPRLLIYRVSNRFSGVPDRFSGSGSGTDFTLKISRVEAEDVGVY | |
| | FCFQGTHVPYTFGGGTKVEIK | |
| 71 | DVVMTQSPLSLPVTLGQPASISCRSSQSLVHSYGNTYLYWFQQRP | VL or LC |
| | GQSPRLLIYRVSNRFSGVPDRFSGSGSGTDFTLKISRVEAEDVGVY | |
| | YCFQGTHVPYTFGGGTKVEIK | |
| 72 | EVQLVQSGAEVKKPGESLRISCKASGYSFTSYWVHWVRQMPGK | VH or HC |
| | GLEWMGNIDPSDSDTHYNQKFKDHVTLSVDKSISTAYLQLSSLK | |
| | ASDTAMYYCVRGGTGTMAWFAYWGQGTLVTVSS | |
| 73 | EVQLVQSGAEVKKPGESLRISCKASGYSFTSYWVHWVRQMPGK | VH or HC |
| | GLEWMGNIDPSDSDTHYSPSFQGHVTLSVDKSISTAYLQLSSLKA | |
| | SDTAMYYCVRGGTGTMAWFAYWGQGTLVTVSS | |
| 74 | EVQLVQSGAEVKKPGESLRISCKASGYSFTSYWVHWVRQMPGK | VH or HC |
| | GLEWMGNIDPSDSDTHYSPSFQGHVTLSVDKSISTAYLQLSSLKA | |
| | SDTAMYYCVRGGTGTLAWFAYWGQGTLVTVSS | |
| 75 | EVQLVQSGAEVKKPGESLRISCKASGYSFTSYWVHWVRQMPGK | VH or HC |
| | GLEWMGNIDPSDSDTHYSPSFQGHVTLSVDKSISTAYLQLSSLKA | |
| | SDTAMYYCVRGGTGTMAYFAYWGQGTLVTVSS | |
| 76 | EVQLVQSGAEVKKPGESLRISCKASGYSFTSYWVHWVRQMPGK | VH or HC |
| | GLEWMGNIDPSDSDTHYSPSFQGHVTLSVDKSISTAYLQLSSLKA | |
| | SDTAMYYCVRGGTGTLAYFAYWGQGTLVTVSS | |
| 77 | MWPLVAALLLGSACCGSAQLLFNKTKSVEFTFCNDTVVIPCFVTN | CD47-201 |
| | MEAQNTTEVYVKWKFKGRDIYTFDGALNKSTVPTDFSSAKIEVS | |
| | QLLKGDASLKMDKSDAVSHTGNYTCEVTELTREGETIIELKYRVV | |
| | SWFSPNENILIVIFPIFAILLFWGQFGIKTLKYRSGGMDEKTIALLV | |
| | AGLVITVIVIVGAILFVPGEYSLKNATGLGLIVTSTGILILLHYYVFS | |
| | TAIGLTSFVIAILVIQVIAYILAVVGLSLCIAACIPMHGPLLISGLSIL | |
| | ALAQLLGLVYMKFVASNQKTIQPPRNN | |
| 78 | MWPLVAALLLGSACCGSAQLLFNKTKSVEFTFCNDTVVIPCFVTN | CD47-202 |
| | MEAQNTTEVYVKWKFKGRDIYTFDGALNKSTVPTDFSSAKIEVS | |
| | QLLKGDASLKMDKSDAVSHTGNYTCEVTELTREGETIIELKYRVV | |
| | SWFSPNENILIVIFPIFAILLFWGQFGIKTLKYRSGGMDEKTIALLV | |
| | AGLVITVIVIVGAILFVPGEYSLKNATGLGLIVTSTGILILLHYYVFS | |
| | TAIGLTSFVIAILVIQVIAYILAVVGLSLCIAACIPMHGPLLISGLSIL | |
| | ALAQLLGLVYMKFVASNQKTIQPPRKAVEEPLNAFKESKGMMN | |
| | DE | |
| 79 | MEAQNTTEVYVKWKFKGRDIYTFDGALNKSTVPTDFSSAK | CD47-206 |
| | IEVSQLLKGDASLKMDKSDAVSHTGNYTCEVTELTREGET | |
| | IIELKYRVVSWFSPNENILIVIFPIFAILLFWGQFGIKTL | |
| | KYRSGGMDEKTIALLVAGLVITVIVIVGAILFVPGEYSLK | |
| | NATGLGLIVTSTGILILLHYYVF | |

What is claimed:

1. A method of treating or preventing a beta cell disorder in a subject in need thereof, the method comprising administering to the subject a dose of engineered hypoimmunogenic islets,
wherein the dose is administered to the subject via intramuscular injection, and
wherein the dose is a dose from:
A) about 1×10⁷ cells to about 3×10⁸ cells;
B) about 1.25×10⁵ cells/kg to about 1.2×10⁷ cells/kg;
C) about 6,500 islet equivalents (IEQ) to about 600,000 IEQ; or
D) about 80 IEQ/kg to about 24,000 IEQ/kg.

2. A method of reducing exogenous insulin dependence in a subject having or at risk of having a beta cell disorder, the method comprising administering to the subject a dose of engineered hypoimmunogenic islets,
wherein the dose is administered via intramuscular injection,
wherein the dose is a dose from:
A) about 1×10⁷ cells to about 3×10⁷ cells;
B) about 1.25×10⁵ cells/kg to about 1.2×10⁷ cells/kg;
C) about 6,500 islet equivalents (IEQ) to about 600,000 IEQ; or
D) about 80 IEQ/kg to about 24,000 IEQ/kg, and
wherein the amount of exogenous insulin required is less than the amount of exogenous insulin required for a subject treated with non-hypoimmunogenic islets or is less than the amount of exogenous insulin required for untreated subjects that have the beta cell disorder.

3. A method of stabilizing glucose levels in a subject having or at risk of having a beta cell disorder, the method comprising administering to the subject a dose of engineered hypoimmunogenic islets,
wherein the dose is administered via intramuscular injection,
wherein the dose is a dose from:
A) about 1×10⁷ cells to about 3×10⁸ cells;
B) about 1.25×10⁵ cells/kg to about 1.2×10⁷ cells/kg;
C) about 6,500 islet equivalents (IEQ) to about 600,000 IEQ; or
D) about 80 IEQ/kg to about 24,000 IEQ/kg,
wherein the glucose levels are stabilized compared to a subject administered an alternative islet therapy or compared to an untreated subject.

4. A method of stabilizing/increasing c-peptide levels in a subject having or at risk of having a beta cell disorder, the method comprising administering to the subject a dose of engineered hypoimmunogenic islets,
wherein the dose is administered via intramuscular injection, and
wherein the dose is a dose from:
A) about 1×10⁷ cells to about 3×10⁸ cells;
B) about 1.25×10⁵ cells/kg to about 1.2×10⁷ cells/kg;
C) about 6,500 islet equivalents (IEQ) to about 600,000 IEQ; or
D) about 80 IEQ/kg to about 24,000 IEQ/kg,
wherein the c-peptide levels are stabilized or increased compared to a subject administered an alternative islet therapy or compared to an untreated subject.

5. A method of reducing HbA1c levels in a subject having or at risk of having a beta cell disorder, the method comprising administering to the subject a dose of engineered hypoimmunogenic islets,
wherein the dose is administered via intramuscular injection, and
wherein the dose is a dose from:
A) about 1×10⁷ cells to about 3×10⁷ cells;
B) about 1.25×10⁵ cells/kg to about 1.2×10⁷ cells/kg;
C) about 6,500 islet equivalents (IEQ) to about 600,000 IEQ; or
D) about 80 IEQ/kg to about 24,000 IEQ/kg,
wherein the HbA1c levels are reduced compared to a subject administered an alternative islet therapy or compared to an untreated subject.

6. A method of reducing adverse side effects associated with islet cell therapy in a subject having or at risk of having a beta cell disorder, the method comprising
i) introducing hypoimmunogenic modification to a population of islet cells comprising beta cells to generate engineered hypoimmunogenic islets, and
ii) administering a dose of the engineered hypoimmunogenic islets to a subject having or at risk of having a beta cell disorder,
wherein the dose is administered via intramuscular injection, and
wherein the dose is a dose from:
A) about 1×10⁷ cells to about 3×10⁸ cells;
B) about 1.25×10⁵ cells/kg to about 1.2×10⁷ cells/kg;
C) about 6,500 islet equivalents (IEQ) to about 600,000 IEQ; or
D) about 80 IEQ/kg to about 24,000 IEQ/kg.

7. A method increasing time in range (TIR) in a subject having or at risk of having a beta cell disorder, the method comprising administering to the subject a dose of engineered hypoimmunogenic islets,
wherein the dose is administered via intramuscular injection, and
wherein the dose is a dose from:
A) about 1×10⁷ cells to about 3×10⁷ cells;
B) about 1.25×10⁵ cells/kg to about 1.2×10⁷ cells/kg;
C) about 6,500 islet equivalents (IEQ) to about 600,000 IEQ; or
D) about 80 IEQ/kg to about 24,000 IEQ/kg,
wherein the TIR is increased compared to a subject administered an alternative islet therapy or compared to an untreated subject.

8. The method of any of claims 1-7, wherein the method results in reduction in other medication requirements for treating the beta cell disorder, optionally wherein the beta cell disorder medication is insulin.

9. The method of any of claims 1-8, wherein the subject exhibits reduced insulin dependence.

10. The method of claim 2 or claim 9, wherein the amount of exogenous insulin is reduced by 10%, about 20%, about 30%, about 40%, about 50%, about 60%, about 70%, about 75%, about 80% or more compared to the amount of exogenous insulin required for a subject administered non-hypoimmunogenic islets for treating the beta cell disorder or the amount of exogenous insulin required for untreated subjects that have the beta cell disorder.

11. The method of any of claims 2, 9, and 10, wherein the method is characterized by the subject meeting one or more of the following criteria: (i) fasting capillary glucose level does not exceed 140 mg/dL (7.8 mmol/L) more than three times in 1 week (based on measuring capillary glucose levels a minimum of 7 times in a seven day period); (ii) 2-hours post-prandial capillary glucose does not exceed 180 mg/dL (10.0 mmol/L) more than three times in 1 week (based on measuring capillary glucose levels a minimum of 21 times in a seven day period); and (iii) evidence of endogenous insulin production defined as fasting or stimulated C-peptide levels >0.5 ng/mL (0.16 pmol/L).

12. The method of any of claims 1-6, wherein the method results in the subject exhibiting insulin-independence.

13. The method of claim 11, wherein the subject exhibits insulin-independence for a period of greater than one month, greater than two months, greater than three months, greater than four months, greater than 5 months, greater than 6 months, greater than 7 months, greater than 8 months, greater than 9 months, greater than 10 months, greater than 11 months or greater than 12 months.

14. The method of any of claims 1-13, the method is characterized by the subject meeting one or more of the following:
a) Peak c-peptide >0.20 nmol/l (as assessed by mixed meal tolerance test);
b) Non-fasting c-peptide >0.10 nmol/l (as assessed by mixed meal tolerance test);
c) Daily exogenous insulin requirement <0.25 U/kg;
d) Daily exogenous insulin requirement=0 U/kg;
e) Decrease in exogenous insulin requirement (per kg body weight);
f) Decrease in HbA1c (per kg body weight);
g) Decrease in glucose variability (stabilization);
h) Decrease in duration of hypoglycemia and/or hyperglycemia (improved euglycemia);
i) Glycemic control HbA1c<6.50% (48 mmol/mol); and
j) Glycemic control HbA1c≤7.0% (53 mmol/mol).

15. The method of any of claims 1-14, wherein the engineered hypoimmunogenic islets comprise modifications that:
(a) inactivate or disrupt one or more alleles of: (i) one or more major histocompatibility complex (MHC) class I molecules or one or more molecules that regulate expression of the one or more MHC class I molecules, and/or (ii) one or more MHC class II molecules or one or more molecules that regulate expression of the one or more MHC class II molecules; and/or
(b) increase expression of one or more tolerogenic factors, wherein the increased expression is relative to a control or wild-type islet that does not comprise the modifications.

16. The method of any one of claims 1-15, wherein the engineered hypoimmunogenic islets comprise engineered beta islet cells.

17. The method of claim 16, wherein the engineered hypoimmunogenic islets further comprises additional engineered islet cells, optionally wherein the additional engineered islet cells comprise alpha cells and/or delta cells.

18. The method of claim 17, wherein the additional engineered islet cells comprises cells that comprises the same modifications of the engineered beta islet cells.

19. The method of any one of claims 1-18, wherein at least 20%, at least 25%, at least 30%, at least 35%, at least 40%, at least 45%, at least 50%, at least 55%, at least 60%, at least 65%, or at least 70% of the cells in the engineered hypoimmunogenic islets comprise engineered beta islet cells.

20. The method of any one of claims 1-19, wherein the engineered hypoimmunogenic islets is an islet cluster.

21. The method of any one of claims 1-20, wherein the engineered hypoimmunogenic islets is engineered from primary islets.

22. The method of claim 21, wherein the primary islets are from a pancreas.

23. The method of claim 21 or claim 22, wherein the primary islets are from a human subject or an animal subject, optionally wherein the primary islets are porcine, bovine or ovine.

24. The method of any of claims 22 or 23, wherein the primary islets are from a donor subject that is not suspected of having a beta cell related disorder.

25. The method of claim 24, wherein the donor is a cadaver.

26. The method of any one of claims 1-25, wherein the engineered hypoimmunogenic islets are ABO blood group type O.

27. The method of any one of claims 1-26, wherein the engineered hypoimmunogenic islets are Rhesus factor negative (Rh-).

28. The method of any one of claims 1-27, wherein the engineered hypoimmunogenic islets are differentiated from a stem cell.

29. The method of any one of claims 1-28, wherein the beta cell disorder is diabetes.

30. The method of any one of claims 1-29, wherein the beta cell disorder is Type I diabetes.

31. The method of any of claims 1-30, wherein the subject to be treated is characterized by one or more of the following: type 1 diabetes for more than 5 years, C-peptide negative (or <0.01 nmol/1) in response to mixed meal tolerance test (MMTT), positive for antibodies to either GAD or IA2, HbA_{1c}>70 mmol/mol, and an exogenous insulin requirement <1 U/kg.

32. The method of any one of claims 1-31, wherein the dose of engineered hypoimmunogenic islets comprises a pharmaceutically acceptable carrier.

33. The method of any one of claims 1-32, wherein intramuscular administration is via the intramuscular space of the forearm, upper arm, hip, thigh or buttocks.

34. The method of any one of claims 1-33, wherein the dose comprises administration of one or more further doses of the hypoimmunogenic engineered cells.

35. The method of claim 34, wherein the one or more further doses of the hypoimmunogenic engineered cells is administered to the subject when, after the initial dose:
(a) the subject does not exhibit a reduction in other medication requirements for treating the beta cell disorder, optionally wherein the beta cell disorder medication is insulin; and/or
(b) the administered hypoimmunogenic engineered cells are not detected by imaging.

36. The method of claim 34, wherein the one or more further doses of the hypoimmunogenic engineered cells is administered to the subject when, after the initial dose, the subject does not meet one or more of the following criteria: (i) fasting capillary glucose level does not exceed 140 mg/dL (7.8 mmol/L) more than three times in 1 week (based on measuring capillary glucose levels a minimum of 7 times in a seven day period); (ii) 2-hours post-prandial capillary glucose does not exceed 180 mg/dL (10.0 mmol/L) more than three times in 1 week (based on measuring capillary glucose levels a minimum of 21 times in a seven day period); and (iii) evidence of endogenous insulin production defined as fasting or stimulated C-peptide levels >0.5 ng/mL (0.16 pmol/L).

37. The method of claim 34, wherein the one or more further doses of the hypoimmunogenic engineered cells is administered to the subject when:
(a) the subject does not achieve insulin-independence within a period of time after the initial dose; and/or
(b) the subject does not exhibit a reduction in other medication requirements for treating the beta cell disorder within a period of time, optionally wherein the beta cell disorder medication is insulin,
optionally wherein the subject does not achieve insulin-independence for a period of greater than one week, greater than two weeks, greater than three weeks, greater than one month, greater than two months, greater than three months, greater than four months, greater than 5 months, greater than 6 months, greater than 7 months, greater than 8 months, greater than 9 months, greater than 10 months, greater than 11 months or greater than 12 months, optionally wherein the subject does not achieve insulin-independence for a period of 2 weeks.

38. The method of claim 34, wherein the one or more further doses of the hypoimmunogenic engineered cells is administered to the subject when, after the initial dose, the subject does not meet one or more of the following criteria:
a) Peak c-peptide >0.20 nmol/l (as assessed by mixed meal tolerance test);
b) Non-fasting c-peptide >0.10 nmol/l (as assessed by mixed meal tolerance test);
c) Daily exogenous insulin requirement <0.25 U/kg;
d) Daily exogenous insulin requirement=0 U/kg;
e) Decrease in exogenous insulin requirement (per kg body weight);
f) Decrease in HbA1c (per kg body weight);
g) Decrease in glucose variability (stabilization);
h) Decrease in duration of hypoglycemia and/or hyperglycemia (improved euglycemia);
i) Glycemic control HbA1c<6.50% (48 mmol/mol); and
j) Glycemic control HbA1c≤7.0% (53 mmol/mol).

39. The method of any one of claims 35-38, wherein prior to administering the one or more further doses of engineered hypoimmune islets, the number of the engineered hypoimmunogenic islets from the initial dose are cleared or reduced in the subject.

40. The method of claim 39, wherein the number of engineered hypoimmunogenic islets are reduced in the subject following administration of an exogenously administered agent to direct targeted death of the engineered hypoimmunogenic islets, optionally wherein the exogenously administered agent activates a suicide gene or safety switch in the engineered cells or recognizes one or more tolerogenic factors on the surface of the engineered hypoimmunogenic islets.

41. The method of any one of claims 1-40, wherein the subject is administered an immunosuppression regimen.

42. The method of claim 41, wherein the immunosuppression regimen is administered to the subject only prior to administration of the dose of the engineered hypoimmunogenic islets.

43. The method of claim 41 or 42, wherein the immunosuppression regimen is administered to the subject only after administration of the dose of the engineered hypoimmunogenic islets.

44. The method of any one of claims 41-43, wherein the immunosuppression regimen comprises one or more immunosuppression agents.

45. The method of claim 44, wherein the one or more immunosuppression agents comprise a small molecule or a biological product.

46. The method of claim 45, wherein the biological product is a protein and/or an antibody.

47. The method of claim 45, wherein the small molecule is a chemical compound or a nucleic acid.

48. The method of any one of claims 44-47, wherein the one or more immunosuppression agents are selected from the group consisting of calcineurin inhibitors, steroids, alkylating agents, antibiotics, analgesics, anti-inflammatory agents, antihistamines, antiviral agents, anti-fungal agents, anti-coagulation agents, DNA synthesis inhibitors, anti-coagulation agents, hemorheologic agents, inosine monophosphate dehydrogenase (IMPDH) inhibitors, Janus kinase inhibitors, mTOR inhibitors, TNF inhibitors, and anti-CD25 inhibitors.

49. The method of claim 48, wherein the one or more immunosuppression agents are selected from the group consisting of antithymocyte globulin (ATG), corticosteroids, prednisone, cortisone, prednisolone methylprednisolone, dexamethasone, betamethasone, hydrocortisone, methotrexate, acetaminophen, diphenhydramine, sirolimus (rapamycin), tacrolimus (FK-506), mycophenolic acid (MPA), mycophenolate mofetil (MMF), mycophenolate sodium, cyclosporine, etanercept (TNFR-Fc), azathioprine, gold salts, sulfasalazine, antimalarials, brequinar, leflunomide, mizoribine, 15-deoxyspergualine, 6-mercaptopurine, cyclophosphamide, OKT3, anti-thymocyte globulin, thymopentin (thymosin-α), fludarabine, and an immunosuppressive antibody.

50. The method of any one of claims 44-47, wherein the one or more immunosuppression agents comprise: an antibody for binding to MHC, CD2, CD3, CD4, CD7, CD28, B7, CD25, CD40, CD45, CD95, IFN-gamma, TNF-alpha, IL-2Ralpha, IL-4, IL-5, IL-6R, IL-6, IGF, IGFR1, IL-7, IL-8, IL-10, CD11alpha, or CD58, and antibodies binding to any of their ligands; soluble IL-15R, IL-10, B7 molecules such as B7-1, B7-2, variants thereof, and fragments thereof, ICOS, and OX40; and an inhibitor of a negative T cell regulator, such as an antibody against CTLA-4, or similar agents.

51. The method of any one of claims 1-50, further comprising tapering the administration of the one or more immunosuppression agents.

52. The method of any of claims 15-51, wherein the one or more tolerogenic factors is selected from the group consisting of CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, and MANF.

53. The method of any of claims 15-52, wherein at least one of the one or more tolerogenic factors is CD47.

54. The method of claim 53, wherein the CD47 is an engineered CD47 protein.

55. The method of claim 54, wherein the engineered CD47 protein comprises:
(a) one or more extracellular domains; and
(b) one or more membrane tethers;
wherein the one or more extracellular domains comprise a signal-regulatory protein alpha (SIRPα) interaction motif, and
wherein the engineered protein does not comprise one or more full-length CD47 intracellular domains.

56. The method of claim 55, wherein the SIRPα interaction motif is or comprises a CD47 extracellular domain or a portion thereof.

57. The method of claim 55, wherein the SIRPα interaction motif is or comprises a SIRPα antibody or a portion thereof.

58. The method of any of claims 1-56, wherein the engineered hypoimmunogenic islets has the phenotype B2M^{indeI/indel}; CIITA^{indeI/indel}; CD47tg.

59. The method of any of claims 1-58, wherein the engineered hypoimmunogenic islets exhibits one or more functions of a wild-type or control beta islet cell, optionally wherein the one or more functions is selected from the group consisting of in vitro glucose-stimulated insulin secretion (GSIS), glucose metabolism, maintaining fasting blood glucose levels, secreting insulin in response to glucose injections in vivo, and clearing glucose after a glucose injection in vivo.

60. The method of claim 59, wherein the GSIS is dynamic GSIS comprising first and second phase dynamic insulin secretion.

61. The method of claim 59, wherein the GSIS is static GSIS, optionally wherein the static incubation index is greater than at or about 1, greater than at or about 2, greater than at or about 5, greater than at or about 10 or greater than at or about 20.

62. The method of any of claims 1-61, wherein the level of insulin secretion by the engineered hypoimmunogenic islets is at least 20% of that observed for primary islets, optionally cadaveric islets.

63. The method of any of claims 1-62, wherein the total insulin content of the engineered hypoimmunogenic islets is greater than at or about 500 μIU Insulin per 5000 cells, greater than at or about 1000 μIU Insulin per 5000 cells, greater than at or about 2000 μIU Insulin per 5000 cells, greater than at or about 3000 μIU Insulin per 5000 cells or greater than at or about 4000 μIU Insulin per 5000 cells.

64. The method of any of claims 1-63, wherein the engineered hypoimmunogenic islets exhibits functionality for more than 2 weeks following administration into a subject.

65. The method of any of claims 1-64, wherein the dose is selected from: about 1×10⁷ cells to about 3×10⁷ cells, from about 25×10⁶ cells to about 80×10⁷ cells, from about 25×10⁶ cells to about 25 ×10⁷ cells, from about 80×10⁶ cells to about 80×10⁷ cells, from about 25×10⁶ cells to about 80×10⁶ cells, or from about 1.25×10⁵ cells/kg to about 1.2×10⁷ cells/kg.

66. The method of any of claims 1-65, wherein the dose is selected from about 6,500 islet equivalents (IEQ) to about 600,000 IEQ or from about 80 IEQ/kg to about 24,000 IEQ/kg.

67. The method of any of claims 1-66, wherein the method is characterized by the subject meeting one or more of the following:
a) immune evasion of engineered hypoimmunogenic islets, as evaluated in systemic PBMC and serum;
b) peak c-peptide >0.01 nmol/1 in response to a mixed meal tolerance test (MMTT);
c) non-fasting c-peptide concentration >0.01 nmol/l;
d) survival of engineered hypoimmunogenic islets, as evaluated by MRI;
e) decreases in insulin requirement/kg BW;
f) decreases in HbA1c; and
g) reductions in glucose variability, hypoglycemia, and hyperglycemia.

68. The method of claim 67, wherein the engineered hypoimmunogenic islets demonstrate immune evasion at 0, 2, 4, 8, 12, 18, 26, and 52 weeks following administration to the subject.

69. The method of claim 67, wherein the engineered hypoimmunogenic islets survive 2, 4, 6, 8, 12, 26, and 52 weeks following administration to the subject.

70. The method of claim 67, wherein the insulin requirement/kg of body weight decreases at 1, 2, 3, 4, 6, 8, 10, 12, 14, 16, 18, 20, 22, 24, 26, and 52 weeks following administration of the engineered hypoimmunogenic islets to the subject.

71. The method of claim 67, wherein the HbA1c decreases at 2, 4, 6, 8, 10, 12, 14, 16, 18, 20, 26, and 52 weeks following administration of the engineered hypoimmunogenic islets to the subject.

72. The method of claim 67, wherein glucose variability, hypoglycemia, and hyperglycemia are reduced at 4, 8, 12, 18, 26, and 52 weeks following administration of the engineered hypoimmunogenic islets to the subject.

73. The method of any of claims 1-72, wherein the subject is not characterized by having the following: any previous organ transplantation; any history of malignancy; use of any investigational agent(s) within 4 weeks of receiving the dose of engineered hypoimmunogenic islets; use of any anti-diabetic medication other than insulin within 4 weeks of receiving the dose of engineered hypoimmunogenic islets; active infections including Tuberculosis, HIV, HBV and HCV; liver function test value for AST, ALT, GGT or ALP exceeding the respective reference interval; serological evidence of infection with HTLVI or HTLVII; pregnancy, nursing, intention for pregnancy; chronic kidney disease grade 3 or worse (GFR <60 ml/min as estimated by creatine measurement); medical history of cardiac disease or symptoms at screening consistent with cardiac disease; HLA immunization, MIC A/B immunization; known autoimmune disease other than type I diabetes (e.g., Hashimoto disease); administration of live attenuated vaccines <6 months before receiving the dose of engineered hypoimmunogenic islets; islet antibodies GADA >2000 IE/mL or IA2A >4000 IE/mL or ZnT8 autoantibodies; untreated proliferative diabetic retinopathy; ongoing psychiatric illness; ongoing substance abuse, drug or alcohol or treatment noncompliance; and known hypersensitivity to ciprofloxacin, gentamicin, or amphotericin.


SEQUENCE LISTING (79 sequences)

SEQ ID NO: 1 - protein, 305 aa, Homo sapiens
  REGION 1..305: note = Human CD47; aa 19-323
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 2 - protein, 323 aa, Homo sapiens
  REGION 1..323: note = Human CD47; aa 1-323
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 3 - protein, 358 aa, Homo sapiens
  REGION 1..358: note = Human CD46, aa 35-392
CEEPPTFEAMELIGKPKPYYEIGERVDYKCKKGYFYIPPLATHTICDRNHTWLPVSDDACYRETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKVLPPSSTKPPALSHSVSTSSTTKSPASSASGPRPTYKPPVSNYPGYPKPEEGILDSLDVWVIAVIVIAIVVGVAVICVVPYRYLQRRKKKGTYLTDETHREVKFTSL

SEQ ID NO: 4 - protein, 392 aa, Homo sapiens [sequence omitted]

SEQ ID NO: 5 - protein, 103 aa, Homo sapiens
  REGION 1..103: note = Human CD59; aa 26-128
LQCYNCPNPTADCKTAVNCSSDFDACLITKAGLQVYNKCWKFEHCNFNDVTTRLRENELTYYCCKKDLCNFNEQLENGGTSLSEKTVLLLVTPFLAAAWSLHP

SEQ ID NO: 6 - protein, 128 aa, Homo sapiens
  REGION 1..128: note = Human CD59; aa 1-128
MGIQGGSVLFGLLLVLAVFCHSGHSLQCYNCPNPTADCKTAVNCSSDFDACLITKAGLQVYNKCWKFEHCNFNDVTTRLRENELTYYCCKKDLCNFNEQLENGGTSLSEKTVLLLVTPFLAAAWSLHP

SEQ ID NO: 7 - protein, 77 aa, Homo sapiens
  REGION 1..77: note = Mature Human CD59; aa 26-102
LQCYNCPNPTADCKTAVNCSSDFDACLITKAGLQVYNKCWKFEHCNFNDVTTRLRENELTYYCCKKDLCNFNEQLEN

SEQ ID NO: 8 - protein, 347 aa, Homo sapiens
  REGION 1..347: note = Human CD55; aa 35-381
DCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGHTCFTLTGLLGTLVTMGLLT

SEQ ID NO: 9 - protein, 381 aa, Homo sapiens
  REGION 1..381: note = Human CD55; aa 1-381
MTVARPSVPAALPLLGELPRLLLLVLLCLPAVWGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGHTCFTLTGLLGTLVTMGLLT

SEQ ID NO: 10 - protein, 319 aa, Homo sapiens
  REGION 1..319: note = Mature Human CD55; aa 35-353
DCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTS

SEQ ID NO: 11 - protein, 18 aa, synthetic construct
  REGION 1..18: note = T2A
EGRGSLLTCGDVEENPGP

SEQ ID NO: 12 - protein, 24 aa, synthetic construct
  REGION 1..24: note = T2A
LEGGGEGRGSLLTCGDVEENPGPR

SEQ ID NO: 13 - protein, 22 aa, synthetic construct
  REGION 1..22: note = P2A
GSGATNFSLLKQAGDVEENPGP

SEQ ID NO: 14 - protein, 19 aa, synthetic construct
  REGION 1..19: note = P2A
ATNFSLLKQAGDVEENPGP

SEQ ID NO: 15 - protein, 20 aa, synthetic construct
  REGION 1..20: note = E2A
QCTNYALLKLAGDVESNPGP

SEQ ID NO: 16 - protein, 22 aa, synthetic construct
  REGION 1..22: note = F2A
VKQTLNFDLLKLAGDVESNPGP

SEQ ID NO: 17 - protein, 21 aa, synthetic construct
  REGION 1..21: note = T2A
GSGEGRGSLLTCGDVEENPGP

SEQ ID NO: 18 - protein, 23 aa, synthetic construct
  REGION 1..23: note = T2A
AAGSGEGRGSLLTCGDVEENPGP

SEQ ID NO: 19 - RNA, 12 nt, synthetic construct
  source 1..12: mol_type = other RNA
  misc_feature 1..12: note = Exemplary crRNA repeat
gttttagagcta

SEQ ID NO: 20 - RNA, 63 nt, synthetic construct
  source 1..63: mol_type = other RNA
  misc_feature 1..63: note = Exemplary tracrRNA
tagcaagttaaaataaggctagtccgttatcaacttgaaaaagtggcaccgagtcggtgcttt

SEQ ID NO: 21 - skipped

SEQ ID NO: 22 - RNA, 20 nt, synthetic construct
  source 1..20: mol_type = other RNA
  misc_feature 1..20: note = CCR5 gRNA
accccacagtggggccacta

SEQ ID NO: 23 - RNA, 20 nt, synthetic construct
  source 1..20: mol_type = other RNA
  misc_feature 1..20: note = CCR5 gRNA
tgttggaaggatgaggaaat

SEQ ID NO: 24 - RNA, 20 nt, synthetic construct
  source 1..20: mol_type = other RNA
  misc_feature 1..20: note = CCR5 gRNA
tcactatgctgccgcccagt

SEQ ID NO: 25 - RNA, 20 nt, synthetic construct
  source 1..20: mol_type = other RNA
  misc_feature 1..20: note = ABO gRNA
tctctccatgtgcagtagga

SEQ ID NO: 26 - RNA, 20 nt, synthetic construct
  source 1..20: mol_type = other RNA
  misc_feature 1..20: note = 1..20
ctggatgtcggaggagtacg

SEQ ID NO: 27 - RNA, 20 nt, synthetic construct
  source 1..20: mol_type = other RNA
  misc_structure 1..20: note = RHD gRNA
gtctccggaaactcgaggtg

SEQ ID NO: 28 - RNA, 20 nt, synthetic construct
  source 1..20: mol_type = other RNA
  misc_feature 1..20: note = F3 (CD142) gRNA
acagtgtagacttgattgac

SEQ ID NO: 29 - RNA, 20 nt, synthetic construct
  source 1..20: mol_type = other RNA
  misc_feature 1..20: note = B2M gene gRNA sequence
cgtgagtaaacctgaatctt

SEQ ID NO: 30 - RNA, 20 nt, synthetic construct
  source 1..20: mol_type = other RNA
  misc_feature 1..20: note = CIITA gene gRNA sequence
gatattggcataagcctccc

SEQ ID NO: 31 - RNA, 20 nt, synthetic construct
  source 1..20: mol_type = other RNA
  misc_feature 1..20: note = TRAC gRNA
agagtctctcagctggtaca

SEQ ID NO: 32-34 - skipped (3 entries)

SEQ ID NO: 35 - protein, 18 aa, synthetic construct
  REGION 1..18: note = CD47 SP domain
MWPLVAALLLGSACCGSA

SEQ ID NO: 36 - protein, 109 aa, synthetic construct
  REGION 1..109: note = CD47 IgV domain
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETII

SEQ ID NO: 37 - protein, 123 aa, synthetic construct
  REGION 1..123: note = CD47 EC domain
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNE

SEQ ID NO: 38 - protein, 21 aa, synthetic construct
  REGION 1..21: note = CD47 TM1 domain
NILIVIFPIFAILLFWGQFGI

SEQ ID NO: 39 - protein, 14 aa, synthetic construct
  REGION 1..14: note = CD47 IC1 domain
KTLKYRSGGMDEKT

SEQ ID NO: 40 - protein, 21 aa, synthetic construct
  REGION 1..21: note = CD47 TM2 domain
IALLVAGLVITVIVIVGAILF

SEQ ID NO: 41 - protein, 10 aa, synthetic construct
  REGION 1..10: note = CD47 EC1 domain
VPGEYSLKNA

SEQ ID NO: 42 - protein, 21 aa, synthetic construct
  REGION 1..21: note = CD47 TM3 domain
TGLGLIVTSTGILILLHYYVF

SEQ ID NO: 43 - protein, 7 aa, synthetic construct
  REGION 1..7: note = CD47 IC2 domain
STAIGLT

SEQ ID NO: 44 - protein, 21 aa, synthetic construct
  REGION 1..21: note = CD47 TM4 domain
SFVIAILVIQVIAYILAVVGL

SEQ ID NO: 45 - protein, 12 aa, synthetic construct
  REGION 1..12: note = CD47 EC2 domain
SLCIAACIPMHG

SEQ ID NO: 46 - protein, 21 aa, synthetic construct
  REGION 1..21: note = CD47 TM5 domain
PLLISGLSILALAQLLGLVYM

SEQ ID NO: 47 - protein, 34 aa, synthetic construct
  REGION 1..34: note = CD47(202) IC3 domain
KFVASNQKTIQPPRKAVEEPLNAFKESKGMMNDE

SEQ ID NO: 48 - protein, 16 aa, synthetic construct
  REGION 1..16: note = CD47(201) IC3 Domain
KFVASNQKTIQPPRNN

SEQ ID NO: 49 - protein, 37 aa, synthetic construct
  REGION 1..37: note = DAF/CD55 GPI
PNKGSGTTSGTTRLLSGHTCFTLTGLLGTLVTMGLLT

SEQ ID NO: 50 - protein, 103 aa, synthetic construct
  REGION 1..103: note = TRAILR3 GPI Long (with TAPE repeats)
ATVETPAAEETMNTSPGTPAPAAEETMNTSPGTPAPAAEETMTTSPGTPAPAAEETMTTSPGTPAPAAEETMITSPGTPASSHYLSCTIVGIIVLIVLLIVFV

SEQ ID NO: 51 - protein, 32 aa, synthetic construct
  REGION 1..32: note = TRAILR3 GPI Short (without TAPE repeats)
MITSPGTPASSHYLSCTIVGIIVLIVLLIVFV

SEQ ID NO: 52 - protein, 30 aa, synthetic construct
  REGION 1..30: note = CD59 GPI
QLENGGTSLSEKTVLLLVTPFLAAAWSLHP

SEQ ID NO: 53 - protein, 21 aa, synthetic construct
  REGION 1..21: note = CD8a TM domain
IYIWAPLAGTCGVLLLSLVIT

SEQ ID NO: 54 - protein, 9 aa, synthetic construct
  REGION 1..9: note = CD8a 9AA ICD
LYCNHRNRR

SEQ ID NO: 55 - protein, 27 aa, synthetic construct
  REGION 1..27: note = CD28 TM domain
FWVLVVVGGVLACYSLLVTVAFIIFWV

SEQ ID NO: 56 - protein, 9 aa, synthetic construct
  REGION 1..9: note = CD28 9AA ICD
RSKRSRLLH

SEQ ID NO: 57 - protein, 21 aa, synthetic construct
  REGION 1..21: note = PDGFR TM domain
VVISAILALVVLTIISLIILI

SEQ ID NO: 58 - protein, 9 aa, synthetic construct
  REGION 1..9: note = PDGFR 9AA ICD
MLWQKKPRY

SEQ ID NO: 59 - skipped

SEQ ID NO: 60 - protein, 45 aa, synthetic construct
  REGION 1..45: note = CD8a Hinge domain
TTTPAPRPPTPAPTIASQPLSLRPEACRPAAGGAVHTRGLDFACD

SEQ ID NO: 61 - protein, 39 aa, synthetic construct
  REGION 1..39: note = CD28 Hinge domain
IEVMYPPPYLDNEKSNGTIIHVKGKHLCPSPLFPGPSKP

SEQ ID NO: 62 - protein, 20 aa, synthetic construct
  REGION 1..20: note = PDGFR Hinge domain
AVGQDTQEVIVVPHSLPFKV

SEQ ID NO: 63 - protein, 12 aa, synthetic construct
  REGION 1..12: note = IgG4 Hinge domain
ESKYGPPCPPCP

SEQ ID NO: 64 - protein, 113 aa, synthetic construct
  REGION 1..113: note = VH or HC
EVQLQQSGAELVKPGASVKLSCTASGFNIKDYYIHWVQQRTEQGLEWIGRIDPEDGETKYAPKFQDKATITADTSSNTAYLHLSSLTSEDTAVYYCARWGAYWGQGTLVTVSA

SEQ ID NO: 65 - protein, 108 aa, synthetic construct
  REGION 1..108: note = VL or LC
QIVLTQSPAIMSASPGEKVTLTCSASSSVSSSYLYWYQQKPGSSPKLWIYSTSNLASGVPARFSGSGSGTSYSLTISSMEAEDAASYFCHQWSSYPRTFGAGTKLELK

SEQ ID NO: 66 - protein, 113 aa, synthetic construct
  REGION 1..113: note = VH or HC
EVQLQQSGAELVKPGASVKLSCTASGFNIKDYYIHWVQQRTEQGLEWIGRIDPEDGETKYAPKFQDKATITADTSSNTAYLHLSSLTSEDTAVYYCARWGAYWGQGTLVTVSS

SEQ ID NO: 67 - protein, 235 aa, synthetic construct
  REGION 1..235: note = scFv
EVQLQQSGAELVKPGASVKLSCTASGFNIKDYYIHWVQQRTEQGLEWIGRIDPEDGETKYAPKFQDKATITADTSSNTAYLHLSSLTSEDTAVYYCARWGAYWGQGTLVTVSGGGGSGGGGSGGGGSQIVLTQSPAIMSASPGEKVTLTCSASSSVSSSYLYWYQQKPGSSPKLWIYSTSNLASGVPARFSGSGSGTSYSLTISSMEAEDAASYFCHQWSSYPRTFGAGTKLELK

SEQ ID NO: 68 - protein, 236 aa, synthetic construct
  REGION 1..236: note = scFv
QIVLTQSPAIMSASPGEKVTLTCSASSSVSSSYLYWYQQKPGSSPKLWIYSTSNLASGVPARFSGSGSGTSYSLTISSMEAEDAASYFCHQWSSYPRTFGAGTKLELKGTTAASGSSGGSSSGAEVQLQQSGAELVKPGASVKLSCTASGFNIKDYYIHWVQQRTEQGLEWIGRIDPEDGETKYAPKFQDKATITADTSSNTAYLHLSSLTSEDTAVYYCARWGAYWGQGTLVTVS

SEQ ID NO: 69 - protein, 120 aa, synthetic construct
  REGION 1..120: note = VH or HC
EVQLVQSGAEVKKPGESLRISCKASGYTFTSYWVHWVRQMPGKGLEWIGNIDPSDSDTHYNQKFKDHVTLSVDKSISTAYLQLSSLKASDTAMYYCVRGGTGTMAWFAYWGQGTLVTVSS

SEQ ID NO: 70 - protein, 112 aa, synthetic construct
  REGION 1..112: note = VL or LC
DVVMTQSPLSLPVTLGQPASISCRSSQSLVHSYGNTYLYWYQQRPGQSPRLLIYRVSNRFSGVPDRFSGSGSGTDFTLKISRVEAEDVGVYFCFQGTHVPYTFGGGTKVEIK

SEQ ID NO: 71 - protein, 112 aa, synthetic construct
  REGION 1..112: note = VL or LC
DVVMTQSPLSLPVTLGQPASISCRSSQSLVHSYGNTYLYWFQQRPGQSPRLLIYRVSNRFSGVPDRFSGSGSGTDFTLKISRVEAEDVGVYYCFQGTHVPYTFGGGTKVEIK

SEQ ID NO: 72 - protein, 120 aa, synthetic construct
  REGION 1..120: note = VH or HC
EVQLVQSGAEVKKPGESLRISCKASGYSFTSYWVHWVRQMPGKGLEWMGNIDPSDSDTHYNQKFKDHVTLSVDKSISTAYLQLSSLKASDTAMYYCVRGGTGTMAWFAYWGQGTLVTVSS

SEQ ID NO: 73 - protein, 120 aa, synthetic construct
  REGION 1..120: note = VH or HC
EVQLVQSGAEVKKPGESLRISCKASGYSFTSYWVHWVRQMPGKGLEWMGNIDPSDSDTHYSPSFQGHVTLSVDKSISTAYLQLSSLKASDTAMYYCVRGGTGTMAWFAYWGQGTLVTVSS

SEQ ID NO: 74 - protein, 120 aa, synthetic construct
  REGION 1..120: note = VH or HC
EVQLVQSGAEVKKPGESLRISCKASGYSFTSYWVHWVRQMPGKGLEWMGNIDPSDSDTHYSPSFQGHVTLSVDKSISTAYLQLSSLKASDTAMYYCVRGGTGTLAWFAYWGQGTLVTVSS

SEQ ID NO: 75 - protein, 120 aa, synthetic construct
  REGION 1..120: note = VH or HC
EVQLVQSGAEVKKPGESLRISCKASGYSFTSYWVHWVRQMPGKGLEWMGNIDPSDSDTHYSPSFQGHVTLSVDKSISTAYLQLSSLKASDTAMYYCVRGGTGTMAYFAYWGQGTLVTVSS

SEQ ID NO: 76 - protein, 120 aa, synthetic construct
  REGION 1..120: note = VH or HC
EVQLVQSGAEVKKPGESLRISCKASGYSFTSYWVHWVRQMPGKGLEWMGNIDPSDSDTHYSPSFQGHVTLSVDKSISTAYLQLSSLKASDTAMYYCVRGGTGTLAYFAYWGQGTLVTVSS

SEQ ID NO: 77 - protein, 305 aa, synthetic construct
  REGION 1..305: note = CD247-201
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 78 - protein, 323 aa, synthetic construct; identical to SEQ ID NO: 2
  REGION 1..323: note = CD247-202

SEQ ID NO: 79 - protein, 183 aa, synthetic construct
  REGION 1..183: note = CD47-206
MEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVF